(19) United States
(12) Patent Application Publication
(10) Pub. No.: US 2010/0081792 A1
(43) Pub. Date: Apr. 1, 2010

(54) Ligand

(75) Inventors: Steven Grant, Cambridge (GB); Amrik Basran, Cambridge (GB); Olga Ignatovich, Cambridge (GB); Rudolph Maria T. De Wildt, Cambridge (GB); Philip Jones, Cambridge (GB); Neil Brewis, Cambridge (GB); Ben Woolven, Cambridge (GB); Elena De Angelis, Cambridge (GB); Lucy J. Holt, London (GB); Greg Winter, Cambridge (GB); Ian Tomlinson, Cambridge (GB); Kevin Moulder, Cambridge (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SmithKline Beecham Corporation, Philadelphia, PA (US)

(21) Appl. No.: 11/981,821

(22) Filed: Oct. 31, 2007

Related U.S. Application Data
(63) Continuation-in-part of application No. 11/098,758, filed on Apr. 4, 2005.
Application No. 11/098,758 is a continuation-in-part of application No. 10/925,366, filed on Aug. 24, 2004, now abandoned.
Application No. 10/925,366 is a continuation-in-part of application No. 10/744,774, filed on Dec. 23, 2003.
Application No. 10/744,774 is a continuation-in-part of application No. PCT/GB2002/003014, filed on Jun. 28, 2002.
Application No. 10/925,366 is a continuation-in-part of application No. PCT/GB2003/002804, filed on Jun. 30, 2003.
Application No. PCT/GB2003/002804 is a continuation-in-part of application No. 11/023,959, filed on Dec. 28, 2004.
Application No. 11/023,959 is a continuation of application No. PCT/GB2003/002804, filed on Jun. 30, 2003.
Application No. PCT/GB2003/002804 is a continuation-in-part of application No. 11/141,661, filed on May 31, 2005.
Application No. 11/141,661 is a continuation of application No. PCT/GB2003/005646, filed on Dec. 24, 2003.
(60) Provisional application No. 60/509,613, filed on Oct. 8, 2003.

(30) Foreign Application Priority Data
Jun. 28, 2001 (GB) GB0115841.9
Dec. 27, 2002 (GB) GB0230202.4
Nov. 28, 2003 (GB) GB03227706.8

(51) Int. Cl. C07K 16/00

(52) U.S. Cl. 530/387.1

(57) ABSTRACT

The invention provides a dual-specific ligand comprising a first and second single variable domain, each having binding specificity for a antigenic target. The invention also provides for a single variable domain monomer ligand that specifically binds to an antigenic target.

### BACKGROUND OF THE INVENTION

### Antibody Polypeptides:

Antibodies are highly specific for their binding targets and although they are derived from nature's own defense mechanisms, antibodies face several challenges when applied to the treatment of disease in human patients. Conventional antibodies are large multi-subunit protein molecules comprising at least four polypeptide chains. For example, human IgG has two heavy chains and two light chains that are disulfide bonded to form the functional antibody. The size of a conventional IgG is about 150 kD. Because of their relatively large size, complete antibodies (e.g., IgG, IgA, IgM, etc.) are limited in their therapeutic usefulness due to problems in, for example, tissue penetration. Considerable efforts have focused on identifying and producing smaller antibody fragments that retain antigen binding function and solubility.

The heavy and light polypeptide chains of antibodies comprise variable (V) regions that directly participate in antigen interactions, and constant (C) regions that provide structural support and function in non-antigen-specific interactions with immune effectors. The antigen binding domain of a conventional antibody is comprised of two separate domains: a heavy chain variable domain (V_{H}) and a light chain variable domain (V_{L}: which can be either V_{κ} or V_{λ}). The antigen binding site itself is formed by six polypeptide loops: three from the V_{H} domain (H1, H2 and H3) and three from the V_{L} domain (L1, L2 and L3). In vivo, a diverse primary repertoire of V genes that encode the V_{H} and V_{L} domains is produced by the combinatorial rearrangement of gene segments. C regions include the light chain C regions (referred to as C_{L} regions) and the heavy chain C regions (referred to as C_{H}1, C_{H}2 and C_{H}3 regions).

A number of smaller antigen binding fragments of naturally occurring antibodies have been identified following protease digestion. These include, for example, the “Fab fragment” (V_{L}-C_{L}-C_{H}1-V_{H}), “Fab′ fragment” (a Fab with the heavy chain hinge region) and “F(ab′)₂ fragment” (a dimer of Fab′ fragments joined by the heavy chain hinge region). Recombinant methods have been used to generate even smaller antigen-binding fragments, referred to as “single chain Fv” (variable fragment) or “scFv,” consisting of V_{L} and V_{H} joined by a synthetic peptide linker.

### Single Domain Antibodies:

While the antigen binding unit of a naturally-occurring antibody (e.g., in humans and most other mammals) is generally known to be comprised of a pair of V regions (V_{L}/V_{H}), camelid species express a large proportion of fully functional, highly specific antibodies that are devoid of light chain sequences. The camelid heavy chain antibodies are found as homodimers of a single heavy chain, dimerized via their constant regions. The variable domains of these camelid heavy chain antibodies are referred to as V_{H}H domains and retain the ability, when isolated as fragments of the V_{H} chain, to bind antigen with high specificity ((Hamers-Casterman et al., 1993, Nature 363: 446-448; Gahroudi et al., 1997, FEBS Lett. 414: 521-526). Antigen binding single V_{H} domains have also been identified from, for example, a library of murine V_{H} genes amplified from genomic DNA from the spleens of immunized mice and expressed in E. coli (Ward et al., 1989, Nature 341: 544-546). Ward et al. named the isolated single V_{H} domains “dAbs,” for “domain antibodies.” The term “dAb” will refer herein to a single immunoglobulin variable domain (V_{H}, V_{HH} or V_{L}) polypeptide that specifically binds antigen. A “dAb” binds antigen independently of other V domains; however, as the term is used herein, a “dAb” can be present in a homo- or heteromultimer with other V_{H} or V_{L} domains where the other domains are not required for antigen binding by the dAb, i.e., where the dAb binds antigen independently of the additional V_{H}, V_{HH} or V_{L} domains.

Single immunoglobulin variable domains, for example, V_{H}H, are the smallest antigen-binding antibody unit known. For use in therapy, human antibodies are preferred, primarily because they are not as likely to provoke an immune response when administered to a patient. As noted above, isolated non-camelid V_{H} domains tend to be relatively insoluble and are often poorly expressed. Comparisons of camelid V_{H}H with the V_{H} domains of human antibodies reveals several key differences in the framework regions of the camelid V_{H}H domain corresponding to the V_{H}/V_{L} interface of the human V_{H} domains. Mutation of these residues of human V_{H}3 to more closely resemble the V_{H}H sequence (specifically Gly 44→Glu, Leu 45→Arg and Trp 47→Gly) has been performed to produce “camelized” human V_{H} domains that retain antigen binding activity (Davies & Riechmann, 1994, FEBS Lett. 339: 285-290) yet have improved expression and solubility. (Variable domain amino acid numbering used herein is consistent with the Kabat numbering convention (Kabat et al., 1991, Sequences of Immunological Interest, 5^{th} ed. U.S. Dept. Health & Human Services, Washington, D.C.)) WO 03/035694 (Muyldermans) reports that the Trp 103→Arg mutation improves the solubility of non-camelid V_{H} domains. Davies & Riechmann (1995, Biotechnology N.Y. 13: 475-479) also report production of a phage-displayed repertoire of camelized human V_{H} domains and selection of clones that bind hapten with affinities in the range of 100-400 nM, but clones selected for binding to protein antigen had weaker affinities.

Single variable domain polypeptide have been described previously by the present inventors in, for example, Published U.S. applications: US20040219643; US20050271663; US20060073141; US20060106203, US20060257406; US20060002935; US20070104710; US20070003549; and pending U.S. applications: U.S. Ser. No. 11/791,781, 371(c) date May 29, 2007; U.S. Ser. No. 11/791,399, 371(c) date Jul. 3, 2007; U.S. Ser. No. 11/628,149, 371(c) date Feb. 2, 2007; U.S. Ser. No. 11/667,393, 371(c) date Jul. 13, 2007; and U.S. Ser. No. 11/664,542, 371(c) date Sep. 6, 2007, the contents of which are incorporated herein by reference in their entirety.

The antigen binding domain of an antibody comprises two separate regions: a heavy chain variable domain (V_{H}) and a light chain variable domain (V_{L}: which can be either V_{κ} or V_{λ}). The antigen binding site itself is formed by six polypeptide loops: three from V_{H} domain (H1, H2 and H3) and three from V_{L} domain (L1, L2 and L3). A diverse primary repertoire of V genes that encode the V_{H} and V_{L} domains is produced by the combinatorial rearrangement of gene segments. The V_{H} gene is produced by the recombination of three gene segments, V_{H}, D and J_{H}. In humans, there are approximately 51 functional V_{H} segments (Cook and Tomlinson (1995) Immunol Today, 16: 237), 25 functional D segments (Corbett et al. (1997) J. Mol. Biol., 268: 69) and 6 functional J_{H} segments (Ravetch et al. (1981) Cell, 27: 583), depending on the haplotype. The V_{H} segment encodes the region of the polypeptide chain which forms the first and second antigen binding loops of the V_{H} domain (H1 and H2), whilst the V_{H}, D and J_{H} segments combine to form the third antigen binding loop of the V_{H} domain (H3). The V_{L} gene is produced by the recombination of only two gene segments, V_{L} and J_{L}. In humans, there are approximately 40 functional V_{κ} segments (Schäble and Zachau (1993) Biol. Chem. Hoppe-Seyler, 374: 1001), 31 functional V_{λ} segments (Williams et al. (1996) J. Mol. Biol., 264: 220; Kawasaki et al. (1997) Genome Res., 7: 250), 5 functional J_{κ} segments (Hieter et al. (1982) J. Biol. Chem., 257: 1516) and 4 functional J_{λ} segments (Vasicek and Leder (1990) J. Exp. Med., 172: 609), depending on the haplotype. The V_{L} segment encodes the region of the polypeptide chain which forms the first and second antigen binding loops of the V_{L} domain (L1 and L2), whilst the V_{L} and J_{L} segments combine to form the third antigen binding loop of the V_{L} domain (L3). Antibodies selected from this primary repertoire are believed to be sufficiently diverse to bind almost all antigens with at least moderate affinity. High affinity antibodies are produced by “affinity maturation” of the rearranged genes, in which point mutations are generated and selected by the immune system on the basis of improved binding.

Analysis of the structures and sequences of antibodies has shown that five of the six antigen binding loops (H1, H2, L1, L2, L3) possess a limited number of main-chain conformations or canonical structures (Chothia and Lesk (1987) J. Mol. Biol., 196: 901; Chothia et al. (1989) Nature, 342: 877). The main-chain conformations are determined by (i) the length of the antigen binding loop, and (ii) particular residues, or types of residue, at certain key position in the antigen binding loop and the antibody framework. Analysis of the loop lengths and key residues has enabled us to the predict the main-chain conformations of H1, H2, L1, L2 and L3 encoded by the majority of human antibody sequences (Chothia et al. (1992) J. Mol. Biol., 227: 799; Tomlinson et al. (1995) EMBO J., 14: 4628; Williams et al. (1996) J. Mol. Biol., 264: 220). Although the H3 region is much more diverse in terms of sequence, length and structure (due to the use of D segments), it also forms a limited number of main-chain conformations for short loop lengths which depend on the length and the presence of particular residues, or types of residue, at key positions in the loop and the antibody framework (Martin et al. (1996) J. Mol. Biol., 263: 800; Shirai et al. (1996) FEBS Letters, 399: 1.

Bispecific antibodies comprising complementary pairs of V_{H} and V_{L} regions are known in the art. These bispecific antibodies must comprise two pairs of V_{H} and V_{L}s, each V_{H}/V_{L} pair binding to a single antigen or epitope. Methods described involve hybrid hybridomas (Milstein & Cuello A C, Nature 305:537-40), minibodies (Hu et al., (1996) Cancer Res 56:3055-3061;), diabodies (Holliger et al., (1993) Proc. Natl. Acad. Sci. USA 90, 6444-6448; WO 94/13804), chelating recombinant antibodies (CRAbs; (Neri et al., (1995) J. Mol. Biol. 246, 367-373), biscFv (e.g. Atwell et al., (1996) Mol. Immunol. 33, 1301-1312), “knobs in holes” stabilised antibodies (Carter et al., (1997) Protein Sci. 6, 781-788). In each case each antibody species comprises two antigen-binding sites, each fashioned by a complementary pair of V_{H} and V_{L} domains. Each antibody is thereby able to bind to two different antigens or epitopes at the same time, with the binding to EACH antigen or epitope mediated by a V_{H} and its complementary V_{L} domain. Each of these techniques presents its particular disadvantages; for instance in the case of hybrid hybridomas, inactive V_{H}/V_{L} pairs can greatly reduce the fraction of bispecific IgG. Furthermore, most bispecific approaches rely on the association of the different V_{H}/V_{L} pairs or the association of V_{H} and V_{L} chains to recreate the two different V_{H}/V_{L} binding sites. It is therefore impossible to control the ratio of binding sites to each antigen or epitope in the assembled molecule and thus many of the assembled molecules will bind to one antigen or epitope but not the other. In some cases it has been possible to engineer the heavy or light chains at the sub-unit interfaces (Carter et al., 1997) in order to improve the number of molecules which have binding sites to both antigens or epitopes but this never results in all molecules having binding to both antigens or epitopes.

There is some evidence that two different antibody binding specificities might be incorporated into the same binding site, but these generally represent two or more specificities that correspond to structurally related antigens or epitopes or to antibodies that are broadly cross-reactive.. For example, cross-reactive antibodies have been described, usually where the two antigens are related in sequence and structure, such as hen egg white lysozyme and turkey lysozyme (McCafferty et al., WO 92/01047) or to free hapten and to hapten conjugated to carrier (Griffiths A D et al. EMBO J 1994 13:14 3245-60). In a further example, WO 02/02773 (Abbott Laboratories) describes antibody molecules with “dual specificity”. The antibody molecules referred to are antibodies raised or selected against multiple antigens, such that their specificity spans more than a single antigen. Each complementary V_{H}/V_{L} pair in the antibodies of WO 02/02773 specifies a single binding specificity for two or more structurally related antigens; the V_{H} and V_{L} domains in such complementary pairs do not each possess a separate specificity. The antibodies thus have a broad single specificity which encompasses two antigens, which are structurally related. Furthermore natural autoantibodies have been described that are polyreactive (Casali & Notkins, Ann. Rev. Immunol. 7, 515-531), reacting with at least two (usually more) different antigens or epitopes that are not structurally related. It has also been shown that selections of random peptide repertoires using phage display technology on a monoclonal antibody will identify a range of peptide sequences that fit the antigen binding site. Some of the sequences are highly related, fitting a consensus sequence, whereas others are very different and have been termed mimotopes (Lane & Stephen, Current Opinion in Immunology, 1993, 5, 268-271). It is therefore clear that a natural four-chain antibody, comprising associated and complementary V_{H} and V_{L} domains, has the potential to bind to many different antigens from a large universe of known antigens. It is less clear how to create a binding site to two given antigens in the same antibody, particularly those which are not necessarily structurally related.

Protein engineering methods have been suggested that may have a bearing on this. For example it has also been proposed that a catalytic antibody could be created with a binding activity to a metal ion through one variable domain, and to a hapten (substrate) through contacts with the metal ion and a complementary variable domain (Barbas et al., 1993 Proc. Natl. Acad. Sci USA 90, 6385-6389). However in this case, the binding and catalysis of the substrate (first antigen) is proposed to require the binding of the metal ion (second antigen). Thus the binding to the V_{H}/V_{L} pairing relates to a single but multi-component antigen.

Methods have been described for the creation of bispecific antibodies from camel antibody heavy chain single domains in which binding contacts for one antigen are created in one variable domain, and for a second antigen in a second variable domain. However the variable domains were not complementary. Thus a first heavy chain variable domain is selected against a first antigen, and a second heavy chain variable domain against a second antigen, and then both domains are linked together on the same chain to give a bispecific antibody fragment (Conrath et al., J. Biol. Chem. 270, 27589-27594). However the camel heavy chain single domains are unusual in that they are derived from natural camel antibodies which have no light chains, and indeed the heavy chain single domains are unable to associate with camel light chains to form complementary V_{H} and V_{L} pairs.

Single heavy chain variable domains have also been described, derived from natural antibodies which are normally associated with light chains (from monoclonal antibodies or from repertoires of domains; see EP-A-0368684). These heavy chain variable domains have been shown to interact specifically with one or more related antigens but have not been combined with other heavy or light chain variable domains to create a ligand with a specificity for two or more different antigens. Furthermore, these single domains have been shown to have a very short in vivo half-life. Therefore such domains are of limited therapeutic value.

It has been suggested to make bispecific antibody fragments by linking heavy chain variable domains of different specificity together (as described above). The disadvantage with this approach is that isolated antibody variable domains may have a hydrophobic interface that normally makes interactions with the light chain and is exposed to solvent and may be “sticky” allowing the single domain to bind to hydrophobic surfaces. Furthermore, in the absence of a partner light chain the combination of two or more different heavy chain variable domains and their association, possibly via their hydrophobic interfaces, may prevent them from binding to one in not both of the ligands they are able to bind in isolation. Moreover, in this case the heavy chain variable domains would not be associated with complementary light chain variable domains and thus may be less stable and readily unfold (Worn & Pluckthun, 1998 Biochemistry 37, 13120-7).

### TNF-α:

As the name implies, Tumor Necrosis Factor-a (TNF-α) was originally described as a molecule having anti-tumor properties, but the molecule was subsequently found to play key roles in other processes, including a prominent role in mediating inflammation and autoimmune disorders. TNF-α is a key proinflammatory cytokine in inflammatory conditions including, for example, rheumatoid arthritis (RA), Crohn's disease, ulcerative colitis and other bowel disorders, psoriasis, toxic shock, graft versus host disease and multiple sclerosis.

The pro-inflammatory actions of TNF-α result in tissue injury, such as inducing procoagulant activity on vascular endothelial cells (Pober, et al., J. Immunol. 136:1680 (1986)), increasing the adherence of neutrophils and lymphocytes (Pober, et al., J. Immunol. 138:3319 (1987)), and stimulating the release of platelet activating factor from macrophages, neutrophils and vascular endothelial cells (Camussi, et al., J. Exp. Med. 166:1390 (1987)).

TNF-α is synthesized as a 26 kD transmembrane precursor protein with an intracellular tail that is cleaved by a TNF-α-converting metalloproteinase enzyme and then secreted as a 17 kD soluble protein. The active form consists of a homotrimer of the 17 kD monomers which interacts with two different cell surface receptors, p55 TNFR1 and p75 TNFR2. There is also evidence that the cell surface bound precursor form of TNF-α can mediate some biological effects of the factor. Most cells express both p55 and p75 receptors which mediate different biological functions of the ligand. The p75 receptor is implicated in triggering lymphocyte proliferation, and the p55 receptor is implicated in TNF-mediated cytotoxicity, apoptosis, antiviral activity, fibroblast proliferation and NF-κB activation (see Locksley et al., 2001, Cell 104: 487-501).

The TNF receptors are members of a family of membrane proteins including the NGF receptor, Fas antigen, CD27, CD30, CD40, Ox40 and the receptor for the lymphotoxin α/β heterodimer. Binding of receptor by the homotrimer induces aggregation of receptors into small clusters of two or three molecules of either p55 or p75. TNF-α is produced primarily by activated macrophages and T lymphocytes, but also by neutrophils, endothelial cells, keratinocytes and fibroblasts during acute inflammatory reactions.

TNF-α is at the apex of the cascade of pro-inflammatory cytokines (Reviewed in Feldmann & Maini, 2001, Ann. Rev. Immunol. 19: 163). This cytokine induces the expression or release of additional proinflammatory cytokines, particularly IL-1 and IL-6 (see, for example, Rutgeerts et al., 2004, Gastroenterology 126: 1593-1610). Inhibition of TNF-α inhibits the production of inflammatory cytokines including IL-1, IL-6, IL-8 and GM-CSF (Brennan et al., 1989, Lancet 2: 244).

Because of its role in inflammation, TNF-α has emerged as an important inhibition target in efforts to reduce the symptoms of inflammatory disorders. Various approaches to inhibition of TNF-□ for the clinical treatment of disease have been pursued, including particularly the use of soluble TNF-α receptors and antibodies specific for TNF-α. Commercial products approved for clinical use include, for example, the antibody products Remicade™ (Infliximab; Centocor, Malvern, Pa.; a chimeric monoclonal IgG antibody bearing human IgG4 constant and mouse variable regions), Humira™ (adalimumab or D2E7; Abbott Laboratories, described in U.S. Pat. No. 6,090,382) and the soluble receptor product Enbrel™ (etanercept, a soluble p75 TNFR2 Fc fusion protein; Immunex).

The role of TNF-α in inflammatory arthritis is reviewed in, for example, Li & Schwartz, 2003, Sringer Semin. Immunopathol. 25: 19-33. In RA, TNF-α is highly expressed in inflamed synovium, particularly at the cartilage-pannus junction (DiGiovine et al., 1988, Ann. Rheum. Dis. 47: 768; Firestein et al., 1990, J. Immunol. 144: 3347; and Saxne et al., 1988, Atrhritis Rheum. 31: 1041). In addition to evidence that TNF-α increases the levels of inflammatory cytokines IL-1, IL-6, IL-8 and GM-CSF, TNF-□ can alone trigger joint inflammation and proliferation of fibroblast-like synoviocytes (Gitter et al., 1989, Immunology 66: 196), induce collagenase, thereby triggering cartilage destruction (Dayer et al., 1985, J. Exp. Med. 162: 2163; Dayer et al., 1986, J. Clin. Invest. 77: 645), inhibit proteoglycan synthesis by articular chondrocytes (Saklatvala, 1986, Nature 322: 547; Saklatvala et al., 1985, J. Exp. Med. 162: 1208) and can stimulate osteoclastogenesis and bone resorption (Abu-Amer et al., 2000, J. Biol. Chem. 275: 27307; Bertolini et al., 1986, Nature 319: 516). TNF-α induces increased release of CD14+ monocytes by the bone marrow. Such monocytes can infiltrate joints and amplify the inflammatory response via the RANK (Receptor Activator or NF-κB)-RANKL signaling pathway, giving rise to osteoclast formation during arthritic inflammation (reviewed in Anandarajah & Richlin, 2004, Curr. Opin. Rheumatol. 16: 338-343).

TNF-α is an acute phase protein which increases vascular permeability through its induction of IL-8, thereby recruiting macrophage and neutrophils to a site of infection. Once present, activated macrophages continue to produce TNF-α, thereby maintaining and amplifying the inflammatory response.

Titration of TNF-α by the soluble receptor construct etanercept is effective for the treatment of RA, but not for treatment of Crohn's disease. In contrast, the antibody TNF-α antagonist infliximab is effective to treat both RA and Crohn's disease. Thus, the mere neutralization of soluble TNF-α is not the only mechanism involved in anti-TNF-based therapeutic efficacy. Rather, the blockade of other pro-inflammatory signals or molecules that are induced by TNF-α also plays a role (Rutgeerts et al., supra). For example, the administration of infliximab apparently decreases the expression of adhesion molecules, resulting in a decreased infiltration of neutrophils to sites of inflammation. Also, infliximab therapy results in the disappearance of inflammatory cells from previously inflamed bowel mucosa in Crohn's disease. This disappearance of activated T cells in the lamina propria is mediated by apoptosis of cells carrying membrane-bound TNF-α following activation of caspases 8, 9 and then 3 in a Fas dependent manner (see Lugering et al., 2001, Gastroenterology 121: 1145-1157). Thus, membrane- or receptor-bound TNF-α is an important target for anti-TNF-α therapeutic approaches. Others have shown that infliximab binds to activated peripheral blood cells and lamina propria cells and induces apoptosis through activation of caspase 3 (see Van den Brande et al., 2003, Gastroenterology 124: 1774-1785).

Intracellularly, the binding of trimeric TNF-α to its receptor triggers a cascade of signaling events, including displacement of inhibitory molecules such as SODD (silencer of death domains) and binding of the adaptor factors FADD, TRADD, TRAF2, c-IAP, RAIDD and TRIP plus the kinase RIP1 and certain caspases (reviewed by Chen & Goeddel, 2002, Science 296: 1634-1635, and by Muzio & Saccani in: Methods in Molecular Medicine: Tumor Necrosis Factor, Methods and Protocols,” Corti and Ghezzi, eds. (Humana Press, New Jersey), pp. 81-99. The assembled signaling complex can activate either a cell survival pathway, through NF-κB activation and subsequent downstream gene activation, or an apoptotic pathway through caspase activation.

Similar extracellular downstream cytokine cascades and intracellular signal transduction pathways can be induced by TNF-α in other diseases. Thus, for other diseases or disorders in which the TNF-α molecule contributes to the pathology, inhibition of TNF-α presents an approach to treatment.

### VEGF:

Angiogenesis plays an important role in the active proliferation of inflammatory synovial tissue. RA synovial tissue, which is highly vascularized, invades the periarticular cartilage and bone tissue and leads to joint destruction.

Vascular endothelial growth factor (VEGF) is the most potent angiogenic cytokine known. VEGF is a secreted, heparin-binding, homodimeric glycoprotein existing in several alternate forms due to alternative splicing of its primary transcript (Leung et al., 1989, Science 246: 1306). VEGF is also known as vascular permeability factor (VPF) due to its ability to induce vascular leakage, a process important in inflammation. The identification of VEGF in synovial tissues of RA patients highlighted the potential role of VEGF in the pathology of RA (Fava et al., 1994, J. Exp. Med. 180: 341: 346; Nagashima et al., 1995, J. Rheumatol. 22: 1624-1630). A role for VEGF in the pathology of RA was solidified following studies in which anti-VEGF antibodies were administered in the murine collagen-induced arthritis (CIA) model. In these studies, VEGF expression in the joints increased upon induction of the disease, and the administration of anti-VEGF antisera blocked the development of arthritic disease and ameliorated established disease (Sone et al., 2001, Biochem. Biophys. Res. Comm. 281: 562-568; Lu et al., 2000, J. Immunol. 164: 5922-5927).

### SUMMARY OF THE INVENTION

The inventors have described, in their copending international patent application WO 03/002609 as well as in copending unpublished UK patent application 0230203.2, dual specific immunoglobulin ligands which comprise immunoglobulin single variable domains where each variable domain may have a different specificity. The domains may act in competition with each other or independently to bind antigens or epitopes on target molecules.

In one configuration, the present invention provides a further improvement in dual specific ligands as developed by the present inventors, in which one specificity of the ligand is directed towards a protein or polypeptide target, and another specificity is directed to a receptor for the target.

Therefore, in a first aspect, the invention provides a dual specific ligand comprising a first dAb specific for a target ligand, and a second dAb specific for a receptor for the target ligand.

Preferably, the dual specific ligand is an open conformation ligand and can bind both the target ligand and the target ligand receptor simultaneously.

Preferred dual specific ligands comprise at least on specificity for TNF alpha and at least one specificity for TNF Receptor 1 (p55). Advantageously, the specificities are provided by one or more dAbs arranged in Fab, F(ab′)₂ or IgG formats. Preferred dAbs are TAR1-5-19 V_{κ} and TAR2h-10-27 V_{H} as set forth below.

The invention may also comprise further modifications and configurations of the dual specific ligands as set forth in the accompanying claims and detailed herein.

Accordingly, in a further aspect, there is provided a dual-specific ligand comprising a first immunoglobulin single variable domain having a binding specificity to a first antigen or epitope and a second complementary immunoglobulin single variable domain having a binding activity to a second antigen or epitope, wherein one or both of said antigens or epitopes acts to increase the half-life of the ligand in vivo and wherein said first and second domains lack mutually complementary domains which share the same specificity, provided that said dual specific ligand does not consist of an anti-HSA V_{H} domain and an anti-β galactosidase V_{κ} domain. Preferably, neither of the first or second variable domains binds to human serum albumin (HSA).

Antigens or epitopes which increase the half-life of a ligand as described herein are advantageously present on proteins or polypeptides found in an organism in vivo. Examples include extracellular matrix proteins, blood proteins, and proteins present in various tissues in the organism. The proteins act to reduce the rate of ligand clearance from the blood, for example by acting as bulking agents, or by anchoring the ligand to a desired site of action. Examples of antigens/epitopes which increase half-life in vivo are given in Annex 1 below.

Increased half-life is useful in in vivo applications of immunoglobulins, especially antibodies and most especially antibody fragments of small size. Such fragments (Fvs, disulphide bonded Fvs, Fabs, scFvs, dAbs) suffer from rapid clearance from the body; thus, whilst they are able to reach most parts of the body rapidly, and are quick to produce and easier to handle, their in vivo applications have been limited by their only brief persistence in vivo. The invention solves this problem by providing increased half-life of the ligands in vivo and consequently longer persistence times in the body of the functional activity of the ligand.

Methods for pharmacokinetic analysis and determination of ligand half-life will be familiar to those skilled in the art. Details may be found in Kenneth, A et al: Chemical Stability of Pharmaceuticals: A Handbook for Pharmacists and in Peters et al, Pharmacokinetc analysis: A Practical Approach (1996). Reference is also made to “Pharmacokinetics”, M Gibaldi & D Perron, published by Marcel Dekker, 2^{nd} Rev. ex edition (1982), which describes pharmacokinetic parameters such as t alpha and t beta half lives and area under the curve (AUC).

Half lives (t 1/2 alpha and t½ beta) and AUC can be determined from a curve of serum concentration of ligand against time. The WinNonlin analysis package (available from Pharsight Corp., Mountain View, Calif. 94040, USA) can be used, for example, to model the curve. In a first phase (the alpha phase) the ligand is undergoing mainly distribution in the patient, with some elimination. A second phase (beta phase) is the terminal phase when the ligand has been distributed and the serum concentration is decreasing as the ligand is cleared from the patient. The t alpha half life is the half life of the first phase and the t beta half life is the half life of the second phase. Thus, advantageously, the present invention provides a ligand or a composition comprising a ligand according to the invention having a to half-life in the range of 15 minutes or more. In one embodiment, the lower end of the range is 30 minutes, 45 minutes, 1 hour, 2 hours, 3 hours, 4 hours, 5 hours, 6 hours, 7 hours, 10 hours, 11 hours or 12 hours. In addition, oralt the range of up to and including 12 hours. In one embodiment, the upper end of the range is 11, 10, 9, 8, 7, 6 or 5 hours. An example of a suitable range is 1 to 6 hours, 2 to 5 hours or 3 to 4 hours.

Advantageously, the present invention provides a ligand or a composition comprising a ligand according to the invention having a tβ half-life in the range of 2.5 hours or more. In one embodiment, the lower end of the range is 3 hours, 4 hours, 5 hours, 6 hours, 7 hours, 10 hours, 11 hours, or 12 hours. In addition, or alternatively, a ligand or composition according to the invention has a tβ half-life in the range of up to and including 21 days. In one embodiment, the upper end of the range is 12 hours, 24 hours, 2 days, 3 days, 5 days, 10 days, 15 days or 20 days. Advantageously a ligand or composition according to the invention will have a tβ half life in the range 12 to 60 hours. In a further embodiment, it will be in the range 12 to 48 hours. In a further embodiment still, it will be in the range 12 to 26 hours.

In addition, or alternatively to the above criteria, the present invention provides a ligand or a composition comprising a ligand according to the invention having an AUC value (area under the curve) in the range of 1 mg.min/ml or more. In one embodiment, the lower end of the range is 5, 10, 15, 20, 30, 100, 200 or 300mg.min/ml. In addition, or alternatively, a ligand or composition according to the invention has an AUC in the range of up to 600 mg.min/ml. In one embodiment, the upper end of the range is 500, 400, 300, 200, 150, 100, 75 or 50 mg.min/ml. Advantageously a ligand according to the invention will have a AUC in the range selected from the group consisting of the following: 15 to 150 mg.min/ml, 15 to 100 mg.min/ml, 15 to 75 mg.min/ml, and 15 to 50 mg.min/ml.

In one embodiment, the dual specific ligand comprises two complementary variable domains, i.e. two variable domains that, in their natural environment, are capable of operating together as a cognate pair or group even if in the context of the present invention they bind separately to their cognate epitopes. For example, the complementary variable domains may be immunoglobulin heavy chain and light chain variable domains (V_{H} and V_{L}). V_{H} and V_{L} domains are advantageously provided by scFv or Fab antibody fragments. Variable domains may be linked together to form multivalent ligands by, for example: provision of a hinge region at the C-terminus of each V domain and disulphide bonding between cysteines in the hinge regions; or provision of dAbs each with a cysteine at the C-terminus of the domain, the cysteines being disulphide bonded together; or production of V-CH & V-CL to produce a Fab format; or use of peptide linkers (for example Gly₄Ser linkers discussed hereinbelow) to produce dimers, trimers and further multimers.

The inventors have found that the use of complementary variable domains allows the two domain surfaces to pack together and be sequestered from the solvent. Furthermore the complementary domains are able to stabilise each other. In addition, it allows the creation of dual-specific IgG antibodies without the disadvantages of hybrid hybridomas as used in the prior art, or the need to engineer heavy or light chains at the sub-unit interfaces. The dual-specific ligands of the first aspect of the present invention have at least one V_{H}/V_{L} pair. A bispecific IgG according to this invention will therefore comprise two such pairs, one pair on each arm of the Y-shaped molecule. Unlike conventional bispecific antibodies or diabodies, therefore, where the ratio of chains used is determinative in the success of the preparation thereof and leads to practical difficulties, the dual specific ligands of the invention are free from issues of chain balance. Chain imbalance in conventional bi-specific antibodies results from the association of two different V_{L} chains with two different V_{H} chains, where V_{L} chain 1 together with V_{H} chain 1 is able to bind to antigen or epitope 1 and V_{L} chain 2 together with V_{H} chain 2 is able to bind to antigen or epitope 2 and the two correct pairings are in some way linked to one another. Thus, only when V_{L} chain 1 is paired with V_{H} chain 1 and V_{L} chain 2 is paired with V_{H} chain 2 in a single molecule is bi-specificity created. Such bi-specific molecules can be created in two different ways. Firstly, they can be created by association of two existing V_{H}/V_{L} pairings that each bind to a different antigen or epitope (for example, in a bi-specific IgG). In this case the V_{H}/V_{L} pairings must come all together in a 1:1 ratio in order to create a population of molecules all of which are bi-specific. This never occurs (even when complementary CH domain is enhanced by “knobs into holes” engineering) leading to a mixture of bi-specific molecules and molecules that are only able to bind to one antigen or epitope but not the other. The second way of creating a bi-specific antibody is by the simultaneous association of two different V_{H} chain with two different V_{L} chains (for example in a bi-specific diabody). In this case, although there tends to be a preference for V_{L} chain 1 to pair with V_{H} chain 1 and V_{L} chain 2 to pair with V_{H} chain 2 (which can be enhanced by “knobs into holes” engineering of the V_{L} and V_{H} domains), this paring is never achieved in all molecules, leading to a mixed formulation whereby incorrect pairings occur that are unable to bind to either antigen or epitope.

Bi-specific antibodies constructed according to the dual-specific ligand approach according to the first aspect of the present invention overcome all of these problems because the binding to antigen or epitope 1 resides within the V_{H} or V_{L} domain and the binding to antigen or epitope 2 resides with the complementary V_{L} or V_{H} domain, respectively. Since V_{H} and V_{L} domains pair on a 1:1 basis all V_{H}/V_{L} pairings will be bi-specific and thus all formats constructed using these V_{H}/V_{L} pairings (Fv, scFvs, Fabs, minibodies, IgGs etc) will have 100% bi-specific activity.

In the context of the present invention, first and second “epitopes” are understood to be epitopes which are not the same and are not bound by a single monospecific ligand. In the first configuration of the invention, they are advantageously on different antigens, one of which acts to increase the half-life of the ligand in vivo. Likewise, the first and second antigens are advantageously not the same.

The dual specific ligands of the invention do not include ligands as described in WO 02/02773. Thus, the ligands of the present invention do not comprise complementary V_{H}/V_{L} pairs which bind any one or more antigens or epitopes co-operatively. Instead, the ligands according to the first aspect of the invention comprise a V_{H}/V_{L} complementary pair, wherein the V domains have different specificities.

Moreover, the ligands according to the first aspect of the invention comprise V_{H}/V_{L} complementary pairs having different specificities for non-structurally related epitopes or antigens. Structurally related epitopes or antigens are epitopes or antigens which possess sufficient structural similarity to be bound by a conventional V_{H}/V_{L} complementary pair which acts in a co-operative manner to bind an antigen or epitope; in the case of structurally related epitopes, the epitopes are sufficiently similar in structure that they “fit” into the same binding pocket formed at the antigen binding site of the V_{H}/V_{L} dimer.

In a further aspect, the present invention provides a ligand comprising a first immunoglobulin variable domain having a first antigen or epitope binding specificity and a second immunoglobulin variable domain having a second antigen or epitope binding specificity wherein one or both of said first and second variable domains bind to an antigen which increases the half-life of the ligand in vivo, and the variable domains are not complementary to one another.

In one embodiment, binding to one variable domain modulates the binding of the ligand to the second variable domain.

In this embodiment, the variable domains may be, for example, pairs of V_{H} domains or pairs of V_{L} domains. Binding of antigen at the first site may modulate, such as enhance or inhibit, binding of an antigen at the second site. For example, binding at the first site at least partially inhibits binding of an antigen at a second site. In such an embodiment, the ligand may for example be maintained in the body of a subject organism in vivo through binding to a protein which increases the half-life of the ligand until such a time as it becomes bound to the second target antigen and dissociates from the half-life increasing protein.

Modulation of binding in the above context is achieved as a consequence of the structural proximity of the antigen binding sites relative to one another. Such structural proximity can be achieved by the nature of the structural components linking the two or more antigen binding sites, eg by the provision of a ligand with a relatively rigid structure that holds the antigen binding sites in close proximity. Advantageously, the two or more antigen binding sites are in physically close proximity to one another such that one site modulates the binding of antigen at another site by a process which involves steric hindrance and/or conformational changes within the immunoglobulin molecule.

The first and the second antigen binding domains may be associated either covalently or non-covalently. In the case that the domains are covalently associated, then the association may be mediated for example by disulphide bonds or by a polypeptide linker such as (Gly₄Ser)ₙ, where n=from 1 to 8, eg, 2, 3, 4, 5 or 7.

Ligands according to the invention may be combined into non-immunoglobulin multi-ligand structures to form multivalent complexes, which bind target molecules with the same antigen, thereby providing superior avidity, while at least one variable domain binds an antigen to increase the half life of the multimer. For example natural bacterial receptors such as SpA have been used as scaffolds for the grafting of CDRs to generate ligands which bind specifically to one or more epitopes. Details of this procedure are described in U.S. Pat. No. 5,831,012. Other suitable scaffolds include those based on fibronectin and Affibodies™. Details of suitable procedures are described in WO 98/58965. Other suitable scaffolds include lipocallin and CTLA4, as described in van den Beuken et al., J. Mol. Biol. (2001) 310, 591-601, and scaffolds such as those described in WO0069907 (Medical Research Council), which are based for example on the ring structure of bacterial GroEL or other chaperone polypeptides.

Protein scaffolds may be combined; for example, CDRs may be grafted on to a CTLA4 scaffold and used together with immunoglobulin V_{H} or V_{L} domains to form a ligand. Likewise, fibronectin, lipocallin and other scaffolds may be combined.

In the case that the variable domains are selected from V-gene repertoires selected for instance using phage display technology as herein described, then these variable domains can comprise a universal framework region, such that is they may be recognised by a specific generic ligand as herein defined. The use of universal frameworks, generic ligands and the like is described in WO99/20749. In the present invention, reference to phage display includes the use of both phage and/or phagemids.

Where V-gene repertoires are used variation in polypeptide sequence is preferably located within the structural loops of the variable domains. The polypeptide sequences of either variable domain may be altered by DNA shuffling or by mutation in order to enhance the interaction of each variable domain with its complementary pair.

In a preferred embodiment of the invention the ‘dual-specific ligand’ is a single chain Fv fragment. In an alternative embodiment of the invention, the ‘dual-specific ligand’ consists of a Fab region of an antibody. The term “Fab region” includes a Fab-like region where two VH or two VL domains are used.

The variable domains may be derived from antibodies directed against target antigens or epitopes. Alternatively they may be derived from a repertoire of single antibody domains such as those expressed on the surface of filamentous bacteriophage. Selection may be performed as described below.

In a further aspect, the invention provides a method for producing a ligand comprising a first immunoglobulin single variable domain having a first binding specificity and a second single immunoglobulin single variable domain having a second (different) binding specificity, one or both of the binding specificities being specific for an antigen which increases the half-life of the ligand in vivo, the method comprising the steps of:
(a) selecting a first variable domain by its ability to bind to a first epitope,
(b) selecting a second variable domain by its ability to bind to a second epitope,
(c) combining the variable domains; and
(d) selecting the ligand by its ability to bind to said first epitope and to said second epitope.

The ligand can bind to the first and second epitopes either simultaneously or, where there is competition between the binding domains for epitope binding, the binding of one domain may preclude the binding of another domain to its cognate epitope. In one embodiment, therefore, step (d) above requires simultaneous binding to both first and second (and possibly further) epitopes; in another embodiment, the binding to the first and second epitoes is not simultaneous.

The epitopes are preferably on separate antigens.

Ligands advantageously comprise V_{H}/V_{L} combinations, or V_{H}/V_{H} or V_{L}/V_{L} combinations of immunoglobulin variable domains, as described above. The ligands may moreover comprise camelid V_{HH} domains, provided that the V_{HH} domain which is specific for an antigen which increases the half-life of the ligand in vivo does not bind Hen egg white lysozyme (HEL), porcine pancreatic alpha-amylase or NmC-A; hcg, BSA-linked RR6 azo dye or S. mutans HG982 cells, as described in Conrath et al., (2001) JBC 276:7346-7350 and WO99/23221, neither of which describe the use of a specificity for an antigen which increases half-life to increase the half life of the ligand in vivo.

In one embodiment, said first variable domain is selected for binding to said first epitope in absence of a complementary variable domain. In a further embodiment, said first variable domain is selected for binding to said first epitope/antigen in the presence of a third variable domain in which said third variable domain is different from said second variable domain and is complementary to the first domain. Similarly, the second domain may be selected in the absence or presence of a complementary variable domain.

The antigens or epitopes targeted by the ligands of the invention, in addition to the half-life enhancing protein, may be any antigen or epitope, but advantageously is an antigen or epitope that is targeted with therapeutic benefit. The invention provides ligands, including open conformation, closed conformation and isolated dAb monomer ligands, specific for any such target, particularly those targets further identified herein. Such targets may be, or be part of, polypeptides, proteins or nucleic acids, which may be naturally occurring or synthetic. In this respect, the ligand of the invention may bind the epitope or antigen and act as an antagonist or agonist (e.g., EPO receptor agonist). One skilled in the art will appreciate that the choice is large and varied. They may be for instance, human or animal proteins, cytokines, cytokine receptors, where cytokine receptors include receptors for the above cytokines, enzymes, co-factors for enzymes or DNA binding proteins. Suitable cytokines and growth factors include, but are preferably not limited to: ApoE, Apo-SAA, BDNF, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER 3 and HER 4, CD4, human chemokine receptors CXCR4 or CCR5, non-structural protein type 3 (NS3) from the hepatitis C virus, TNF-alpha, IgE, IFN-gamma, MMP-12, CEA, H. pylori, TB, influenza, Hepatitis E, MMP-12, internalizing receptors that are over-expressed on certain cells, such as the epidermal growth factor receptor (EGFR), ErBb2 receptor on tumor cells, an internalising cellular receptor, LDL receptor, FGF2 receptor, ErbB2 receptor, transferrin receptor, PDGF receptor, VEGF receptor, PsmAr, an extracellular matrix protein, elastin, fibronectin, laminin, α1-antitrypsin, tissue factor protease inhibitor, PDK1, GSK1, Bad, caspase-9, Forkhead, an antigen of Helicobacter pylori, an antigen of Mycobacterium tuberculosis, and an antigen of influenza virus. It will be appreciated that this list is by no means exhaustive.

In one embodiment of the invention, the variable domains are derived from a respective antibody directed against the antigen or epitope. In a preferred embodiment the variable domains are derived from a repertoire of single variable antibody domains.

In a further aspect, the present invention provides one or more nucleic acid molecules encoding at least a dual-specific ligand as herein defined. The dual specific ligand may be encoded on a single nucleic acid molecule; alternatively, each, domain may be encoded by a separate nucleic acid molecule. Where the ligand is encoded by a single nucleic acid molecule, the domains may be expressed as a fusion polypeptide, in the manner of a scFv molecule, or may be separately expressed and subsequently linked together, for example using chemical linking agents. Ligands expressed from separate nucleic acids will be linked together by appropriate means.

The nucleic acid may further encode a signal sequence for export of the polypeptides from a host cell upon expression and may be fused with a surface component of a filamentous bacteriophage particle (or other component of a selection display system) upon expression.

In a further aspect the present invention provides a vector comprising nucleic acid encoding a dual specific ligand according to the present invention.

In a yet further aspect, the present invention provides a host cell transfected with a vector encoding a dual specific ligand according to the present invention.

Expression from such a vector may be configured to produce, for example on the surface of a bacteriophage particle, variable domains for selection. This allows selection of displayed variable domains and thus selection of ‘dual-specific ligands’ using the method of the present invention.

The present invention further provides a kit comprising at least a dual-specific ligand according to the present invention.

Dual-Specific ligands according to the present invention preferably comprise combinations of heavy and light chain domains. For example, the dual specific ligand may comprise a V_{H} domain and a V_{L} domain, which may be linked together in the form of an scFv. In addition, the ligands may comprise one or more C_{H} or C_{L} domains. For example, the ligands may comprise a C_{H}I domain, C_{H}2 or C_{H}3 domain, and/or a C_{L} domain, Cμ1, Cμ2, Cμ3 or Cμ4 domains, or any combination thereof. A hinge region domain may also be included. Such combinations of domains may, for example, mimic natural antibodies, such as IgG or IgM, or fragments thereof, such as Fv, scFv, Fab or F(ab′)₂ molecules. Other structures, such as a single arm of an IgG molecule comprising V_{H}, V_{L}, C_{H}1 and C_{L} domains, are envisaged.

In a preferred embodiment of the invention, the variable regions are selected from single domain V gene repertoires. Generally the repertoire of single antibody domains is displayed on the surface of filamentous bacteriophage. In a preferred embodiment each single antibody domain is selected by binding of a phage repertoire to antigen.

In a preferred embodiment of the invention each single variable domain may be selected for binding to its target antigen or epitope in the absence of a complementary variable region. In an alternative embodiment, the single variable domains may be selected for binding to its target antigen or epitope in the presence of a complementary variable region. Thus the first single variable domain may be selected in the presence of a third complementary variable domain, and the second variable domain may be selected in the presence of a fourth complementary variable domain. The complementary third or fourth variable domain may be the natural cognate variable domain having the same specificity as the single domain being tested, or a non-cognate complementary domain—such as a “dummy” variable domain.

Preferably, the dual specific ligand of the invention comprises only two variable domains although several such ligands may be incorporated together into the same protein, for example two such ligands can be incorporated into an IgG or a multimeric immunoglobulin, such as IgM. Alternatively, in another embodiment a plurality of dual specific ligands are combined to form a multimer. For example, two different dual specific ligands are combined to create a tetra-specific molecule.

It will be appreciated by one skilled in the art that the light and heavy variable domains of a dual-specific ligand produced according to the method of the present invention may be on the same polypeptide chain, or alternatively, on different polypeptide chains. In the case that the variable domains are on different polypeptide chains, then they may be linked via a linker, generally a flexible linker (such as a polypeptide chain), a chemical linking group, or any other method known in the art.

In a further aspect, the present invention provides a composition comprising a dual-specific ligand, obtainable by a method of the present invention, and a pharmaceutically acceptable carrier, diluent or excipient.

Moreover, the present invention provides a method for the treatment and/or prevention of disease using a ‘dual-specific ligand’ or a composition according to the present invention.

In a second configuration, the present invention provides multispecific ligands which comprise at least two non-complementary variable domains. For example, the ligands may comprise a pair of V_{H} domains or a pair of V_{L} domains. Advantageously, the domains are of non-camelid origin; preferably they are human domains or comprise human framework regions (FWs) and one or more heterologous CDRs. CDRs and framework regions are those regions of an immunoglobulin variable domain as defined in the Kabat database of Sequences of Proteins of Immunological Interest.

Preferred human framework regions are those encoded by germline gene segments DP47 and DPK9. Advantageously, FW1, FW2 and FW3 of a V_{H} or V_{L} domain have the sequence of FW1, FW2 or FW3 from DP47 or DPK9. The human frameworks may optionally contain mutations, for example up to about 5 amino acid changes or up to about 10 amino acid changes collectively in the human frameworks used in the ligands of the invention.

The variable domains in the multispecific ligands according to the second configuration of the invention may be arranged in an open or a closed conformation; that is, they may be arranged such that the variable domains can bind their cognate ligands independently and simultaneously, or such that only one of the variable domains may bind its cognate ligand at any one time.

The inventors have realised that under certain structural conditions, non-complementary variable domains (for example two light chain variable domains or two heavy chain variable domains) may be present in a ligand such that binding of a first epitope to a first variable domain inhibits the binding of a second epitope to a second variable domain, even though such non-complementary domains do not operate together as a cognate pair.

Advantageously, the ligand comprises two or more pairs of variable domains; that is, it comprises at least four variable domains. Advantageously, the four variable domains comprise frameworks of human origin.

In a preferred embodiment, the human frameworks are identical to those of human germline sequences.

The present inventors consider that such antibodies will be of particular use in ligand binding assays for therapeutic and other uses.

Thus, in a first aspect of the second configuration, the present invention provides a method for producing a multispecific ligand comprising the steps of:
a) selecting a first epitope binding domain by its ability to bind to a first epitope,
b) selecting a second epitope binding domain by its ability to bind to a second epitope,
c) combining the epitope binding domains; and
d) selecting the closed conformation multispecific ligand by its ability to bind to said first second epitope and said second epitope.

In a further aspect of the second configuration, the invention provides method for preparing a closed conformation multi-specific ligand comprising a first epitope binding domain having a first epitope binding specificity and a non-complementary second epitope binding domain having a second epitope binding specificity, wherein the first and second binding specificities compete for epitope binding such that the closed conformation multi-specific ligand may not bind both epitopes simultaneously, said method comprising the steps of:
a) selecting a first epitope binding domain by its ability to bind to a first epitope,
b) selecting a second epitope binding domain by its ability to bind to a second epitope,
c) combining the epitope binding domains such that the domains are in a closed conformation; and
d) selecting the closed conformation multispecific ligand by its ability to bind to said first second epitope and said second epitope, but not to both said first and second epitopes simultaneously.

Moreover, the invention provides a closed conformation multi-specific ligand comprising a first epitope binding domain having a first epitope binding specificity and a non-complementary second epitope binding domain having a second epitope binding specificity, wherein the first and second binding specificities compete for epitope binding such that the closed conformation multi-specific ligand may not bind both epitopes simultaneously.

An alternative embodiment of the above aspect of the of the second configuration of the invention optionally comprises a further step (b 1) comprising selecting a third or further epitope binding domain. In this way the multi-specific ligand produced, whether of open or closed conformation, comprises more than two epitope binding specificities. In a preferred aspect of the second configuration of the invention, where the multi-specific ligand comprises more than two epitope binding domains, at least two of said domains are in a closed conformation and compete for binding; other domains may compete for binding or may be free to associate independently with their cognate epitope(s).

According to the present invention the term ‘multi-specific ligand’ refers to a ligand which possesses more than one epitope binding specificity as herein defined.

As herein defined the term ‘closed conformation’ (multi-specific ligand) means that the epitope binding domains of the ligand are attached to or associated with each other, optionally by means of a protein skeleton, such that epitope binding by one epitope binding domain competes with epitope binding by another epitope binding domain. That is, cognate epitopes may be bound by each epitope binding domain individually but not simultaneosuly. The closed conformation of the ligand can be achieved using methods herein described.

“Open conformation” means that the epitope binding domains of the ligand are attached to or associated with each other, optionally by means of a protein skeleton, such that epitope binding by one epitope binding domain does not compete with epitope binding by another epitope binding domain.

As referred to herein, the term ‘competes’ means that the binding of a first epitope to its cognate epitope binding domain is inhibited when a second epitope is bound to its cognate epitope binding domain. For example, binding may be inhibited sterically, for example by physical blocking of a binding domain or by alteration of the structure or environment of a binding domain such that its affinity or avidity for an epitope is reduced.

In a further embodiment of the second configuration of the invention, the epitopes may displace each other on binding. For example, a first epitope may be present on an antigen which, on binding to its cognate first binding domain, causes steric hindrance of a second binding domain, or a coformational change therein, which displaces the epitope bound to the second binding domain.

Advantageously, binding is reduced by 25% or more, advantageously 40%, 50%, 60%, 70%, 80%, 90% or more, and preferably up to 100% or nearly so, such that binding is completely inhibited. Binding of epitopes can be measured by conventional antigen binding assays, such as ELISA, by fluorescence based techniques, including FRET, or by techniques such as surface plasmon resonance which measure the mass of molecules. Specific binding of an antigen-binding protein to an antigen or epitope can be determined by a suitable assay, including, for example, Scatchard analysis and/or competitive binding assays, such as radioimmunoassays (RIA), enzyme immunoassays such as ELISA and sandwich competition assays, and the different variants thereof.

Binding affinity is preferably determined using surface plasmon resonance (SPR) and the BIAcore (Karlsson et al., 1991), using a BIAcore system (Uppsala, Sweden). The BIAcore system uses surface plasmon resonance (SPR, Welford K. 1991, Opt. Quant. Elect. 23:1; Morton and Myszka, 1998, Methods in Enzymology 295: 268) to monitor biomolecular interactions in real time, and uses surface plasmon resonance which can detect changes in the resonance angle of light at the surface of a thin gold film on a glass support as a result of changes in the refractive index of the surface up to 300 nm away. BIAcore analysis conveniently generates association rate constants, dissociation rate constants, equilibrium dissociation constants, and affinity constants. Binding affinity is obtained by assessing the association and dissociation rate constants using a BIAcore™ surface plasmon resonance system (BIAcore, Inc.). A biosensor chip is activated for covalent coupling of the target according to the manufacturer's (BIAcore) instructions. The target is then diluted and injected over the chip to obtain a signal in response units of immobilized material. Since the signal in resonance units (RU) is proportional to the mass of immobilized material, this represents a range of immobilized target densities on the matrix. Dissociation data are fit to a one-site model to obtain k_{off}±s.d. (standard deviation of measurements). Pseudo-first order rate constant (Kd's) are calculated for each association curve, and plotted as a function of protein concentration to obtain kₒₙ±s.e. (standard error of fit). Equilibrium dissociation constants for binding, Kd's, are calculated from SPR measurements as k_{off}/kₒₙ.

As described by Phizicky and Field in Microb. Rev. (1995) 59:114-115, a suitable antigen, such as HSA, is immobilized on a dextran polymer, and a solution containing a ligand for HSA, such as a single variable domain, flows through a cell, contacting the immobilized HSA. The single variable domain retained by immobilized HSA alters the resonance angle of impinging light, resulting in a change in refractive index brought about by increased amounts of protein, i.e. the single variable domain, near the dextran polymer. Since all proteins have the same refractive index and since there is a linear correlation between resonance angle shift and protein concentration near the surface, changes in the protein concentration at the surface due to protein/protein binding can be measured, see Phizicky and Field, supra. To determine a binding constant, the increase in resonance units (RU) is measured as a function of time by passing a solution of single variable domain protein past the immobilized ligand (HSA) until the RU values stabilize, then the decrease in RU is measured as a function of time with buffer lacking the single variable domain. This procedure is repeated at several different concentrations of single variable domain protein. Detailed theoretical background and procedures are described by R. Karlsson, et. al. (991) J. Immunol Methods, 145, 229.

The instrument software produces an equilibrium dissociation constant (Kd) as described above. An equilibrium dissociation constant determined through the use of Surface plasmon resonance is described in U.S. Pat. No. 5,573,957, as being based on a table of dR_{A}/dt and R_{A} values, where R in this example is the HSA/single variable domain complex as measured by the BIAcore in resonance units and where dR/dt is the rate of formation of HSA/single variable domain complexes, i.e. the derivative of the binding curve; plotting the graph dR_{A}/dt vs R_{A} for several different concentrations of single variable domain, and subsequently plotting the slopes of these lines vs. the concentration of single variable domain, the slope of this second graph being the association rate constant (M⁻¹, s⁻¹). The Dissociation Rate Constant or the rate at which the HSA and the single variable domain release from each other, can be determined utilizing the dissociation curve generated on the BIAcore. By plotting and determining the slope of the log of the drop in the response vs time curve, the dissociation rate constant can be measured. The Equilibrium dissociation constant Kd=Dissociation Rate Constant/Association Rate Constant.

According to the method of the present invention, in one embodiment, each epitope binding single variable domain is of a different epitope binding specificity.

In the context of the present invention, first and second “epitopes” are understood to be epitopes which are not the same and are not bound by a single monospecific ligand. They may be on different antigens or on the same antigen, but separated by a sufficient distance that they do not form a single entity that could be bound by a single mono-specific V_{H}/V_{L} binding pair of a conventional antibody. Experimentally, if both of the individual variable domains in single chain antibody form (domain antibodies or dAbs) are separately competed by a monospecific V_{H}/V_{L} ligand against two epitopes then those two epitopes are not sufficiently far apart to be considered separate epitopes according to the present invention.

The closed conformation multispecific ligands of the invention do not include ligands as described in WO 02/02773. Thus, the ligands of the present invention do not comprise complementary V_{H}/V_{L} pairs which bind any one or more antigens or epitopes co-operatively. Instead, the ligands according to the invention preferably comprise non-complementary V_{H}-V_{H} or V_{L}-V_{L} pairs. Advantageously, each V_{H} or V_{L} domain in each V_{H}-V_{H} or V_{L}-V_{L} pair has a different epitope binding specificity, and the epitope binding sites are so arranged that the binding of an epitope at one site competes with the binding of an epitope at another site.

According to the present invention, advantageously, each epitope binding domain comprises an immunoglobulin variable domain. More advantageously, each epitope binding domain will be either a variable light chain domain (V_{L}) or a variable heavy chain domain (V_{H}) of an antibody. In the second configuration of the present invention, the immunoglobulin domains when present on a ligand according to the present invention are non-complementary, that is they do not associate to form a V_{H}/V_{L} antigen binding site. Thus, multi-specific ligands as defined in the second configuration of the invention comprise immunoglobulin domains of the same sub-type, that is either variable light chain domains (V_{L}) or variable heavy chain domains (V_{H}). Moreover, where the ligand according to the invention is in the closed conformation, the immunoglobulin domains may be of the camelid V_{HH} type.

In an alternative embodiment, the ligand(s) according to the invention do not comprise a camelid V_{HH} domain. More particularly, the ligand(s) of the invention do not comprise one or more amino acid residues that are specific to camelid V_{HH} domains as compared to human V_{H} domains.

Advantageously, the single variable domains are derived from antibodies selected for binding activity against different antigens or epitopes. For example, the variable domains may be isolated at least in part by human immunisation. Alternative methods are known in the art, including isolation from human antibody libraries and synthesis of artificial antibody genes.

The variable domains advantageously bind superantigens, such as protein A or protein L. Binding to superantigens is a property of correctly folded antibody variable domains, and allows such domains to be isolated from, for example, libraries of recombinant or mutant domains.

Epitope binding domains according to the present invention comprise a protein scaffold and epitope interaction sites (which are advantageously on the surface of the protein scaffold).

Epitope binding domains may also be based on protein scaffolds or skeletons other than immunoglobulin domains. For example, natural bacterial receptors such as SpA have been used as scaffolds for the grafting of CDRs to generate ligands which bind specifically to one or more epitopes. Details of this procedure are described in U.S. Pat. No. 5,831,012. Other suitable scaffolds include those based on fibronectin and affibodies. Details of suitable procedures are described in WO 98/58965. Other suitable scaffolds include lipocallin and CTLA4, as described in van den Beuken et al., J. Mol. Biol. (2001) 310, 591-601, and scaffolds such as those described in WO0069907 (Medical Research Council), which are based for example on the ring structure of bacterial GroEL or other chaperone polypeptides.

Protein scaffolds may be combined; for example, CDRs may be grafted on to a CTLA4 scaffold and used together with immunoglobulin V_{H} or V_{L} domains to form a multivalent ligand. Likewise, fibronectin, lipocallin and other scaffolds may be combined.

It will be appreciated by one skilled in the art that the epitope binding domains of a closed conformation multispecific ligand produced according to the method of the present invention may be on the same polypeptide chain, or alternatively, on different polypeptide chains. In the case that the variable domains are on different polypeptide chains, then they may be linked via a linker, advantageously a flexible linker (such as a polypeptide chain), a chemical linking group, or any other method known in the art.

The first and the second epitope binding domains may be associated either covalently or non-covalently. In the case that the domains are covalently associated, then the association may be mediated for example by disulphide bonds.

In the second configuration of the invention, the first and the second epitopes are preferably different. They may be, or be part of, polypeptides, proteins or nucleic acids, which may be naturally occurring or synthetic. In this respect, the ligand of the invention may bind an epitope or antigen and act as an antagonist or agonist (e.g., EPO receptor agonist). The epitope binding domains of the ligand in one embodiment have the same epitope specificity, and may for example simultaneously bind their epitope when multiple copies of the epitope are present on the same antigen. In another embodiment, these epitopes are provided on different antigens such that the ligand can bind the epitopes and bridge the antigens. One skilled in the art will appreciate that the choice of epitopes and antigens is large and varied. They may be for instance human or animal proteins, cytokines, cytokine receptors, enzymes co-factors for enzymes or DNA binding proteins. Suitable cytokines and growth factors include but are preferably not limited to: ApoE, Apo-SAA, BDNF, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER 3, HER 4, TACE recognition site, TNF BP-I and TNF BP-II, CD4, human chemokine receptors CXCR4 or CCR5, non-structural protein type 3 (NS3) from the hepatitis C virus, TNF-alpha, IgE, IFN-gamma, MMP-12, CEA, H. pylori, TB, influenza, Hepatitis E, MMP-12, internalising receptors are over-expressed on certain cells, such as the epidermal growth factor receptor (EGFR), ErBb2 receptor on tumor cells, an internalising cellular receptor, LDL receptor, FGF2 receptor, ErbB2 receptor, transferrin receptor, PDGF receptor, VEGF receptor, PsmAr, an extracellular matrix protein, elastin, fibronectin, laminin, α1-antitrypsin, tissue factor protease inhibitor, PDK1, GSK1, Bad, caspase-9, Forkhead, an of an antigen of Helicobacter pylori, an antigen of Mycobacterium tuberculosis, and an antigen of influenza virus, as well as any target disclosed in Annex 2 or Annex 3 hereto, whether in combination as set forth in the Annexes, in a different combination or individually. Cytokine receptors include receptors for the above cytokines, e.g. IL-1 R1; IL-6R; IL-10R; IL-18R, as well as receptors for cytokines set forth in Annex 2 or Annex 3 and also receptors disclosed in Annex 2 and 3. It will be appreciated that this list is by no means exhaustive. Where the multispecific ligand binds to two epitopes (on the same or different antigens), the antigen(s) may be selected from this list.

Advantageously, dual specific ligands may be used to target cytokines and other molecules which cooperate synergistically in therapeutic situations in the body of an organism. The invention therefore provides a method for synergising the activity of two or more cytokines, comprising administering a dual specific ligand capable of binding to said two or more cytokines. In this aspect of the invention, the dual specific ligand may be any dual specific ligand, including a ligand composed of complementary and/or non-complementary domains, a ligand in an open conformation, and a ligand in a closed conformation. For example, this aspect of the invention relates to combinations of V_{H} domains and V_{L} domains, V_{H} domains only and V_{L} domains only.

Synergy in a therapeutic context may be achieved in a number of ways. For example, target combinations may be therapeutically active only if both targets are targeted by the ligand, whereas targeting one target alone is not therapeutically effective. In another embodiment, one target alone may provide some low or minimal therapeutic effect, but together with a second target the combination provides a synergistic increase in therapeutic effect.

Preferably, the cytokines bound by the dual specific ligands of this aspect of the invention are selected from the list shown in Annex 2.

Moreover, dual specific ligands may be used in oncology applications, where one specificity targets CD89, which is expressed by cytotoxic cells, and the other is tumour specific. Examples of tumour antigens which may be targeted are given in Annex 3.

In one embodiment of the second configuration of the invention, the variable domains are derived from an antibody directed against the first and/or second antigen or epitope. In a preferred embodiment the variable domains are derived from a repertoire of single variable antibody domains. In one example, the repertoire is a repertoire that is not created in an animal or a synthetic repertoire. In another example, the single variable domains are not isolated (at least in part) by animal immunisation. Thus, the single domains can be isolated from a naïve library.

The second configuration of the invention, in another aspect, provides a multi-specific ligand comprising a first epitope binding domain having a first epitope binding specificity and a non-complementary second epitope binding domain having a second epitope binding specificity. The first and second binding specificities may be the same or different.

In a further aspect, the present invention provides a closed conformation multi-specific ligand comprising a first epitope binding domain having a first epitope binding specificity and a non-complementary second epitope binding domain having a second epitope binding specificity wherein the first and second binding specificities are capable of competing for epitope binding such that the closed conformation multi-specific ligand cannot bind both epitopes simultaneously.

In a still further aspect, the invention provides open conformation ligands comprising non-complementary binding domains, wherein the domains are specific for a different epitope on the same target. Such ligands bind to targets with increased avidity. Similarly, the invention provides multivalent ligands comprising non-complementary binding domains specific for the same epitope and directed to targets which comprise multiple copies of said epitope, such as IL-5, PDGF-AA, PDGF-BB, TGF beta, TGF beta2, TGF beta3 and TNFα, for example, as well as human TNF Receptor 1 and human TNFα.

In a similar aspect, ligands according to the invention can be configured to bind individual epitopes with low affinity, such that binding to individual epitopes is not therapeutically significant; but the increased avidity resulting from binding to two epitopes provides a therapeutic benefit. In a particular example, epitopes may be targeted which are present individually on normal cell types, but present together only on abnormal or diseased cells, such as tumour cells. In such a situation, only the abnormal or diseased cells are effectively targeted by the bispecific ligands according to the invention.

Ligand specific for multiple copies of the same epitope, or adjacent epitopes, on the same target (known as chelating dAbs) may also be trimeric or polymeric (tertrameric or more) ligands comprising three, four or more non-complementary binding domains. For example, ligands may be constructed comprising three or four V_{H} domains or V_{L} domains.

Moreover, ligands are provided which bind to multisubunit targets, wherein each binding domain is specific for a subunit of said target. The ligand may be dimeric, trimeric or polymeric.

Preferably, the multi-specific ligands according to the above aspects of the invention are obtainable by the method of the first aspect of the invention.

According to the above aspect of the second configuration of the invention, advantageously the first epitope binding domain and the second epitope binding domains are non-complementary immunoglobulin variable domains, as herein defined. That is either V_{H}-V_{H} or V_{L}-V_{L} variable domains.

Chelating dAbs in particular may be prepared according to a preferred aspect of the invention, namely the use of anchor dAbs, in which a library of dimeric, trimeric or multimeric dAbs is constructed using a vector which comprises a constant dAb upstream or downstream of a linker sequence, with a repertoire of second, third and further dAbs being inserted on the other side of the linker. For example, the anchor or guiding dAb may be TAR1-5 (Vκ), TART1-27(Vκ), TAR2h-5(VH) or TAR2h-6(Vκ).

In alternative methodologies, the use of linkers may be avoided, for example by the use of non-covalent bonding or natural affinity between binding domains such as V_{H} and V_{κ}. The invention accordingly provides a method for preparing a chelating multimeric ligand comprising the steps of:

(a) providing a vector comprising a nucleic acid sequence encoding a single binding domain specific for a first epitope on a target;

(b) providing a vector encoding a repertoire comprising second binding domains specific for a second epitope on said target, which epitope can be the same or different to the first epitope, said second epitope being adjacent to said first epitope; and

(c) expressing said first and second binding domains; and

(d) isolating those combinations of first and second binding domains which combine together to produce a target-binding dimer.

The first and second epitopes are adjacent such that a multimeric ligand is capable of binding to both epitopes simultaneously. This provides the ligand with the advantages of increased avidity if binding. Where the epitopes are the same, the increased avidity is obtained by the presence of multiple copies of the epitope on the target, allowing at least two copies to be simultaneously bound in order to obtain the increased avidity effect.

The binding domains may be associated by several methods, as well as the use of linkers. For example, the binding domains may comprise cys residues, avidin and streptavidin groups or other means for non-covalent attachment post-synthesis; those combinations which bind to the target efficiently will be isolated. Alternatively, a linker may be present between the first and second binding domains, which are expressed as a single polypeptide from a single vector, which comprises the first binding domain, the linker and a repertoire of second binding domains, for instance as described above.

In a preferred aspect, the first and second binding domains associate naturally when bound to antigen; for example, V_{H} and VL (e.g. Vκ) domains, when bound to adjacent epitopes, will naturally associate in a three-way interaction to form a stable dimer. Such associated proteins can be isolated in a target binding assay. An advantage of this procedure is that only binding domains which bind to closely adjacent epitopes, in the correct conformation, will associate and thus be isolated as a result of their increased avidity for the target.

In an alternative embodiment of the above aspect of the second configuration of the invention, at least one epitope binding domain comprises a non-immunoglobulin ‘protein scaffold’ or ‘protein skeleton’ as herein defined. Suitable non-immunoglobulin protein scaffolds include but are not limited to any of those selected from the group consisting of: SpA, fibronectin, GroEL and other chaperones, lipocallin, CCTLA4 and affibodies, as set forth above.

According to the above aspect of the second configuration of the invention, advantageously, the epitope binding domains are attached to a ‘protein skeleton’. Advantageously, a protein skeleton according to the invention is an immunoglobulin skeleton.

According to the present invention, the term ‘immunoglobulin skeleton’ refers to a protein which comprises at least one immunoglobulin fold and which acts as a nucleus for one or more epitope binding domains, as defined herein.

Preferred immunoglobulin skeletons as herein defined includes any one or more of those selected from the following: an immunoglobulin molecule comprising at least (i) the CL (kappa or lambda subclass) domain of an antibody; or (ii) the CH1 domain of an antibody heavy chain; an immunoglobulin molecule comprising the CH1 and CH2 domains of an antibody heavy chain; an immunoglobulin molecule comprising the CH1, CH2 and CH3 domains of an antibody heavy chain; or any of the subset (ii) in conjunction with the CL (kappa or lambda subclass) domain of an antibody. A hinge region domain may also be included. Such combinations of domains may, for example, mimic natural antibodies, such as IgG or IgM, or fragments thereof, such as Fv, scFv, Fab or F(ab′)₂ molecules. Those skilled in the art will be aware that this list is not intended to be exhaustive.

Linking of the skeleton to the epitope binding domains, as herein defined may be achieved at the polypeptide level, that is after expression of the nucleic acid encoding the skeleton and/or the epitope binding domains. Alternatively, the linking step may be performed at the nucleic acid level. Methods of linking a protein skeleton according to the present invention, to the one or more epitope binding domains include the use of protein chemistry and/or molecular biology techniques which will be familiar to those skilled in the art and are described herein.

Advantageously, the closed conformation multispecific ligand may comprise a first domain capable of binding a target molecule, and a second domain capable of binding a molecule or group which extends the half-life of the ligand. For example, the molecule or group may be a bulky agent, such as HSA or a cell matrix protein. As used herein, the phrase “molecule or group which extends the half-life of a ligand” refers to a molecule or chemical group which, when bound by a dual-specific ligand as described herein increases the in vivo half-life of such dual specific ligand when administered to an animal, relative to a ligand that does not bind that molecule or group. Examples of molecules or groups that extend the half-life of a ligand are described hereinbelow. In a preferred embodiment, the closed conformation multispecific ligand may be capable of binding the target molecule only on displacement of the half-life enhancing molecule or group. Thus, for example, a closed conformation multispecific ligand is maintained in circulation in the bloodstream of a subject by a bulky molecule such as HSA. When a target molecule is encountered, competition between the binding domains of the closed conformation multispecific ligand results in displacement of the HSA and binding of the target.

A ligand according to any aspect of the present invention, includes a ligand having or consisting of at least one single variable domain, in the form of a monomer single variable domain or in the form of multiple single variable domains, i.e. a multimer. The ligand can be modified to contain additional moieties, such as a fusion protein, or a conjugate. Such a multimeric ligand, e.g., in the form of a dual specific ligand, and/or such a ligand comprising or consisting of a single variable domain, i.e. a dAb monomer useful in constructing such a multimeric ligand, may advantageously dissociate from their cognate target(s) with a Kd of 300 nM or less, 300 nM to 5 pM (i.e., 3×10⁻⁷ to 5×10⁻¹²M), preferably 50 nM to20 pM, or 5 nM to 200 pM or 1 nM to 100 pM, 1×10⁻⁷ M or less, 1×10⁻⁸ M or less, 1×10⁻⁹ M or less, 1×10⁻¹⁰ M or less, 1×10⁻¹¹ M or less; and/or a K_{off} rate constant of 5×10⁻¹ to 1×10⁻⁷S⁻¹, preferably 1×10⁻² to 1×10⁻⁶ S⁻¹, or 5×10⁻³ to 1×10⁻⁵ S⁻¹, or 5×10⁻¹ S⁻¹ or less, or 1×10⁻² S⁻¹ or less, or 5×10⁻³ S⁻¹ or less; 5×10⁻⁴ S⁻¹ or less, or 1×10⁻⁵ S⁻¹ or less, or 1×10⁻⁶ S⁻¹ or less as determined, for example, by surface plasmon resonance. The Kd rate constant is defined as Koff/Kon. A Kd value greater than 1 Molar is generally considered to indicate non-specific binding. Preferably, a single variable domain will specifically bind a target antigen or epitope with an affinity of less than 500 nM, preferably less than 200 nM, and more preferably less than 10 nM, such as less than 500 pM

In particular the invention provides an anti-TNFα dAb monomer (or dual specific ligand comprising such a dAb), homodimer, heterodimer or homotrimer ligand, wherein each dAb binds TNFα. The ligand binds to TNFα with a K_{d} of 300 nM to 5 pM (ie, 3×10⁻⁷ to 5×10⁻¹²M), preferably 50 nM to 20 pM, more preferably 5 nM to 200 pM and most preferably 1 nM to 100 pM; expressed in an alternative manner, the K_{d} is 1×10⁻⁷ M or less, preferably 1×10⁻⁸ M or less, more preferably 1×10⁻⁹ M or less, advantageously 1×10⁻¹⁰ M or less and most preferably 1×10⁻¹¹ M or less; and/or a K_{off} rate constant of 5×10⁻¹ to 1×10⁻⁷S⁻¹, preferably 1×10⁻² to 1×10⁻⁶ S⁻¹, more preferably 5×10⁻³ to 5×10⁻⁵ S⁻¹, for example 5×10⁻¹ S⁻¹ or less, preferably 1×10⁻² S⁻¹ or less, more preferably 1×10⁻³ S⁻¹ or less, advantageously 1×10⁻⁴ S⁻¹ or less, further advantageously 1×10⁻⁵ S^{-and most preferably} 1×10⁻⁶S⁻¹ or less, as determined by surface plasmon resonance.

Preferably, the ligand neutralises TNFα in a standard L929 assay with an ND50 of 500 nM to 50 pM, preferably or 100 nM to 50 pM, advantageously 10 nM to 100 pM, more preferably 1 nM to 100 pM; for example 50 nM or less, preferably 5 nM or less, advantageously 500 pM or less, more preferably 200 pM or less and most preferably 100 pM or less.

Preferably, the ligand inhibits binding of TNF alpha to TNF alpha Receptor I (p55 receptor) with an IC50 of 500 nM to 50 pM, preferably 100 nM to 50 pM, more preferably 10 nM to 100 pM, advantageously 1 nM to 100 pM; for example 50 nM or less, preferably 5 nM or less, more preferably 500 pM or less, advantageously 200 pM or less, and most preferably 100 pM or less. Preferably, the TNFα is Human TNFα.

Furthermore, the invention provides a an anti-TNF Receptor I dAb monomer, or dual specific ligand comprising such a dAb, that binds to TNF Receptor I with a K_{d} of 300 nM to 5 pM (ie, 3×10⁻⁷ to 5×10⁻¹²M), preferably 50 nM to 20 pM, more preferably 5 nM to 200 pM and most preferably 1 nM to 100 pM, for example 1×10⁻⁷ M or less, preferably 1×10⁻⁸ M or less, more preferably 1×10⁻⁹ M or less, advantageously 1×10⁻¹⁰ M or less and most preferably 1×10⁻¹¹ M or less; and/or a K_{off} rate constant of 5×10⁻¹ to 1×10⁻⁷ S⁻¹, preferably 1×10⁻² to 1×10⁻⁶ S⁻¹, more preferably 5×10^{example} 5×10⁻¹S⁻¹ or less, preferably 1×10⁻² S⁻¹ or less, advantageously 1×10⁻³ S⁻¹ or less, more preferably 1×10⁻⁴ S^{31 1} or less, still more preferably 1×10⁻⁵S⁻¹ or less, and most

Preferably, the dAb monomer ligand neutralises TNFα in a standard assay (eg, the L929 or HeLa assays described herein) with an ND50 of 500 nM to 50 pM, preferably 100 nM to 50 pM, more preferably 10 nM to 100 pM, advantageously 1 nM to 100 pM; for example 50 nM or less, preferably 5 nM or less, more preferably 500 pM or less, advantageously 200 pM or less, and most preferably 100 pM or less.

Preferably, the dAb monomer or ligand inhibits binding of TNF alpha to TNF alpha Receptor I (p55 receptor) with an IC50 of 500 nM to 50 pM, preferably 100 nM to 50 pM, more preferably 10 nM to 100 pM, advantageously 1 nM to 100 pM; for example 50 nM or less, preferably 5 nM or less, more preferably 500 pM or less, advantageously 200 pM or less, and most preferably 100 pM or less. Preferably, the TNF Receptor I target is Human TNFα.

Furthermore, the invention provides an anti-TNF Receptor I dAb monomer, or dual specific ligand comprising such a dAb, that binds to TNF Receptor I with a Kd of 300 nM to 5pM (i.e., 3×10⁻⁷ to 5×10⁻¹²M), preferably 50 nM to 20 pM, more preferably 5 nM to 200 pM and most preferably 1 nM to 100 pM, for example 1×10⁻⁷ M or less preferably 1×10⁻⁸ M or less, more preferably 1×10⁻⁹ M or less, advantageously 1×10⁻¹⁰ M or less and most preferably 1×10⁻¹¹ M or less; and/or a K_{off} rate constant of 5×10⁻¹ to 1×10⁻⁷ S⁻¹, preferably 1×10⁻² to 1×10⁻⁶ S⁻¹, more preferably 5×10⁻³ to 5×10⁻⁵ S⁻¹, for example 5×10⁻¹ S⁻¹ or less, preferably 1×10⁻² S⁻¹ or less, advantageously 1×10^{-less, more preferably} 1×10⁻⁴ S⁻¹ or less, still more preferably 1×10⁻⁵ S⁻¹ or less, and mos preferably 1×10⁻⁶S⁻¹ or less, preferably as determined by surface plasmon resonance.

Preferably, the dAb monomer ligand neutralises TNFα in a standard assay (e.g., the L929 or HeLa assays described herein) with an ND50 of 500 nM to 50 pM, preferably 100 nM to 50 pM, more preferably 10 nM to 100 pM, advantageously 1 nM to 100 pM; for example 50 nM or less, preferably 5 nM or less, more preferably 500 pM or less, advantageously 200 pM or less, and most preferably 100 pM or less.

Preferably, the dAb monomer or ligand inhibits binding of TNF alpha to TNF alpha Receptor I (p55 receptor) with an IC50 of 500 nM to 50 pM, preferably 100 nM to 50 pM, more preferably 10 nM to 100 pM, advantageously 1 nM to 100 pM; for example 50 nM or less, preferably 5 nM or less, more preferably 500 pM or less, advantageously 200 pM or less, and most preferably 100 pM or less. Preferably, the TNF Receptor I target is Human TNFα.

Furthermore, the invention provides a dAb monomer (or dual specific ligand comprising such a dAb) that binds to serum albumin (SA) with a Kd of 1 nM to 500 μM (i.e., 1×10⁻⁹ to 5×10⁻⁴), preferably 100 nM to 10 μM. Preferably, for a dual specific ligand comprising a first anti-SA dAb and a second dAb to another target, the affinity (e.g. Kd and/or K_{off} as measured by surface plasmon resonance, e.g. using BIACore) of the second dAb for its target is from 1 to 100000 times (preferably 100 to 100000, more preferably 1000 to 100000, or 10000 to 100000 times) the affinity of the first dAb for SA. For example, the first dAb binds SA with an affinity of approximately 10 μM, while the second dAb binds its target with an affinity of 100 pM. Preferably, the serum albumin is human serum albumin (HSA).

In one embodiment, the first dAb (or a dAb monomer) binds SA (e.g., HSA) with a Kd of approximately 50, preferably 70, and more preferably 100, 150 or 200 nM.

The invention moreover provides dimers, trimers and polymers of the aforementioned dAb monomers, in accordance with the above aspect of the present invention.

Ligands according to the invention, including dAb monomers, dimers and trimers, can be linked to an antibody Fc region, comprising one or both of C_{H}2 and C_{H}3 domains, and optionally a hinge region. For example, vectors encoding ligands linked as a single nucleotide sequence to an Fc region may be used to prepare such polypeptides.

In a further aspect of the second configuration of the invention, the present invention provides one or more nucleic acid molecules encoding at least a multispecific ligand as herein defined. In one embodiment, the multispecific ligand is a closed conformation ligand. In another embodiment, it is an open conformation ligand. The multispecific ligand may be encoded on a single nucleic acid molecule; alternatively, each epitope binding domain may be encoded by a separate nucleic acid molecule.

Where the multispecific ligand is encoded by a single nucleic acid molecule, the domains may be expressed as a fusion polypeptide, or may be separately expressed and subsequently linked together, for example using chemical linking agents. Ligands expressed from separate nucleic acids will be linked together by appropriate means.

The nucleic acid may further encode a signal sequence for export of the polypeptides from a host cell upon expression and may be fused with a surface component of a filamentous bacteriophage particle (or other component of a selection display system) upon expression. Leader sequences, which may be used in bacterial expression and/or phage or phagemid display, include pelB, stII, ompA, phoA, bla and pelA.

In a further aspect of the second configuration of the invention the present invention provides a vector comprising nucleic acid according to the present invention.

In a yet further aspect, the present invention provides a host cell transfected with a vector according to the present invention.

Expression from such a vector may be configured to produce, for example on the surface of a bacteriophage particle, epitope binding domains for selection. This allows selection of displayed domains and thus selection of ‘multispecific ligands’ using the method of the present invention.

In a preferred embodiment of the second configuration of the invention, the epitope binding domains are immunoglobulin variable domains and are selected from single domain V gene repertoires. Generally the repertoire of single antibody domains is displayed on the surface of filamentous bacteriophage. In a preferred embodiment each single antibody domain is selected by binding of a phage repertoire to antigen.

The present invention further provides a kit comprising at least a multispecific ligand according to the present invention, which may be an open conformation or closed conformation ligand. Kits according to the invention may be, for example, diagnostic kits, therapeutic kits, kits for the detection of chemical or biological species, and the like.

In a further aspect still of the second configuration of the invention, the present invention provides a homogenous immunoassay using a ligand according to the present invention.

In a further aspect still of the second configuration of the invention, the present invention provides a composition comprising a closed conformation multispecific ligand, obtainable by a method of the present invention, and a pharmaceutically acceptable carrier, diluent or excipient.

Moreover, the present invention provides a method for the treatment of disease using a ‘closed conformation multispecific ligand’ or a composition according to the present invention.

In a preferred embodiment of the invention the disease is cancer or an inflammatory disease, e.g. rheumatoid arthritis, asthma or Crohn's disease.

In a further aspect of the second configuration of the invention, the present invention provides a method for the diagnosis, including diagnosis of disease using a closed conformation multispecific ligand, or a composition according to the present invention. Thus in general the binding of an analyte to a closed conformation multispecific ligand may be exploited to displace an agent, which leads to the generation of a signal on displacement. For example, binding of analyte (second antigen) could displace an enzyme (first antigen) bound to the antibody providing the basis for an immunoassay, especially if the enzyme were held to the antibody through its active site.

Thus in a final aspect of the second configuration, the present invention provides a method for detecting the presence of a target molecule, comprising:

(a) providing a closed conformation multispecific ligand bound to an agent, said ligand being specific for the target molecule and the agent, wherein the agent which is bound by the ligand leads to the generation of a detectable signal on displacement from the ligand;

(b) exposing the closed conformation multispecific ligand to the target molecule; and

(c) detecting the signal generated as a result of the displacement of the agent.

According to the above aspect of the second configuration of the invention, advantageously, the agent is an enzyme, which is inactive when bound by the closed conformation multi-specific ligand. Alternatively, the agent may be any one or more selected from the group consisting of the following: the substrate for an enzyme, and a fluorescent, luminescent or chromogenic molecule which is inactive or quenched when bound by the ligand.

Sequences similar or homologous (e.g., at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., very high stringency hybridization conditions), to the complement of the strand. The nucleic acids may be present in whole cells, in a cell lysate, or in a partially purified or substantially pure form.

The percent identity can refer to the percent identity along the entire stretch of the length of the amino acid or nucleotide sequence. When specified, the percent identity of the amino acid or nucleic acid sequence refers to the percent identity to sequence(s) from one or more discrete regions of the referenced amino acid or nucleic acid sequence, for instance, along one or more antibody CDR regions, and/or along one or more antibody variable framework regions. For example, the sequence identity at the amino acid level across one or more CDRs of an antibody heavy or light chain single variable domain can have about 80%, 85%, 90%, 91%, 92%, 93%, 94%, 95%, 96%, 97%, 98%, 99% or higher identity to the amino acid sequence of corresponding CDRs of an antibody heavy or light chain single variable domain, respectively. At the nucleic acid level, the nucleic acid sequence encoding one or more CDRs of an antibody heavy or light chain single variable domain can have at least 70%, 75%, 80%, 85%, 90%, 91%, 92%, 93%, 94%, 95%, 96%, 97%, 98%, 99% or higher, identity to the nucleic acid sequence encoding the corresponding CDRs of an antibody heavy or light chain single variable domain. At the nucleic acid level, the nucleic acid sequence encoding one CDR of an antibody heavy or light chain single variable domain can have a percent identity of at least 80%, 85%, 90%, 91%, 92%, 93%, 94%, 95%, 96%, 97%, 98%, 99% or higher, than the nucleic acid sequence encoding the corresponding CDR of an antibody heavy or light chain single variable domain, respectively. In some embodiments, the structural characteristic of percent identity is coupled to a functional aspect. For instance, in some embodiments, a nucleic acid sequence or amino acid sequence with less than 100% identity to a referenced nucleic acid or amino acid sequence is also required to display at least one functional aspect of the reference amino acid sequence or of the amino acid sequence encoded by the referenced nucleic acid. In other embodiments, a nucleic acid sequence or amino acid sequence with less than 100% identity to a referenced nucleic acid or amino acid sequence, respectively, is also required to display at least one functional aspect of the reference amino acid sequence or of the amino acid sequence encoded by the referenced nucleic acid, but that functional characteristic can be slightly altered, e.g., confer an increased affinity to a specified antigen relative to that of the reference.

Calculations of “homology” or “sequence identity” or “similarity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences.

Advantageously, the BLAST algorithm (version 2.0) is employed for sequence alignment, with parameters set to default values. The BLAST algorithm is described in detail at the world wide web site (“www”) of the National Center for Biotechnology Information (“NCBI”) of the National Institutes of Health (“NIH”) of the U.S. government (“gov”), in the “/Blast/” directory, in the “blast_help.html” file. The search parameters are defined as follows, and are advantageously set to the defined default parameters.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul, 1990, Proc. Natl. Acad. Sci. USA 87(6):2264-8 (see the “blast_help.html” file, as described above) with a few enhancements. The BLAST programs were tailored for sequence similarity searching, for example to identify homologues to a query sequence. The programs are not generally useful for motif-style searching. For a discussion of basic issues in similarity searching of sequence databases, see Altschul et al. (1994).

The five BLAST programs available at the National Center for Biotechnology Information web site perform the following tasks:

“blastp” compares an amino acid query sequence against a protein sequence database;

“blastn” compares a nucleotide query sequence against a nucleotide sequence database;

“blastx” compares the six-frame conceptual translation products of a nucleotide query sequence (both strands) against a protein sequence database;

“tblastn” compares a protein query sequence against a nucleotide sequence database dynamically translated in all six reading frames (both strands).

“tblastx” compares the six-frame translations of a nucleotide query sequence against the six-frame translations of a nucleotide sequence database.

BLAST uses the following search parameters:

HISTOGRAM Display a histogram of scores for each search; default is yes. (See parameter H in the BLAST Manual).

DESCRIPTIONS Restricts the number of short descriptions of matching sequences reported to the number specified; default limit is 100 descriptions. (See parameter V in the manual page). See also EXPECT and CUTOFF.

ALIGNMENTS Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

EXPECT The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

MATRIX Specify an alternate scoring matrix for BLASTP, BLASTX, TBLASTN and TBLASTX. The default matrix is BLOSUM62 (Henikoff & Henikoff, 1992, Proc. Natl. Aacad. Sci. USA 89(22):10915-9). The valid alternative choices include: PAM40, PAM120, PAM250 and IDENTITY. No alternate scoring matrices are available for BLASTN; specifying the MATRIX directive in BLASTN requests returns an error response.

STRAND Restrict a TBLASTN search to just the top or bottom strand of the database sequences; or restrict a BLASTN, BLASTX or TBLASTX search to just reading frames on the top or bottom strand of the query sequence.

FILTER Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States, 1993, Computers and Chemistry 17:191-201, or, for BLASTN, by the DUST program of Tatusov and Lipman (see the world wide web site of the NCBI). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “N” repeated 13 times) and the letter “X” in protein sequences (e.g., “X” repeated 9 times).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi Causes NCBI gi identifiers to be shown in the output, in addition to the accession and/or locus name.

Most preferably, sequence comparisons are conducted using the simple BLAST search algorithm provided at the NCBI world wide web site described above, in the “/BLAST” directory.

According to a further aspect the present invention provides a dual specific ligand comprising a first single immunoglobulin variable domain having a binding specificity to a first antigen or epitope and a second immunoglobulin single variable domain having a binding activity to a second antigen or epitope wherein said first and second domains lack mutually complementary domains which share the same specificity.

According to the above aspect of the invention, preferably a dual-specific ligand has an IgG format which comprises two complementary pairs of mammalian dAbs wherein each Dab comprising each complementary pair has a different target binding specificity. Advantageously a dual specific molecule according to this embodiment of the invention comprises one or more Dabs which exhibits an epitope binding specificity of 50 nM or more.

According to the above aspect of the invention the two different dAbs may be both VH domains, both VL domains or at least one VH and a VL domain.

According to the above aspect of the invention, preferably the dual-specific ligand comprises at least one pair of Dabs which are complementary to one another.

Preferably a dual-specific ligand having an IgG format as described binds to its respective targets in a non-competitive manner.

In an alternative embodiment of the above aspect of the invention, a dual-specific ligand having an IgG format as described above binds to its respective targets in a competitive manner.

Advantageously a dual-specific ligand according to the above aspect of the invention has an IgG format and comprises one pair of identical dAbs which can bind simultaneously to two copies of the corresponding target.

More advantageously, a dual-specific ligand according to the above aspect of the invention comprise two pairs of identical dAbs wherein both pairs of identical dAbs can bind simultaneously to two copies of the corresponding targets

Advantageously, a dual-specific ligand according to the above aspect of the invention comprises 4 identical dAbs, preferably mammalian Dabs.

In an alternative embodiment of the above aspect of the invention, a dual-specific molecule according to the invention has a Fab format.

Advantageously, a dual-specific ligand having a Fab format as herein described binds to its respective targets in a non-competitive manner.

In an alternative embodiment of the above aspect of the invention, a dual-specific ligand having a Fab format as described above binds to its respective targets in a competitive manner.

Suitable targets for the dual-specific ligands according to the aspect of the invention described above include any one or more of those in the list consisting of the following: One skilled in the art will appreciate that the choice of epitopes and antigens is large and varied. They may be for instance human or animal proteins, cytokines, cytokine receptors, enzymes co-factors for enzymes or DNA binding proteins. Suitable cytokines and growth factors include but are not limited to: ApoE, Apo-SAA, BDNF, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER 3, HER 4, TACE recognition site, TNF BP-I and TNF BP-II, CD4, human chemokine receptors CXCR4 or CCR5, non-structural protein type 3 (NS3) from the hepatitis C virus, TNF-alpha, IgE, IFN-gamma, MMP-12, CEA, H. pylori, TB, influenza, Hepatitis E, MMP-12, internalizing receptors that are over-expressed on certain cells, such as the epidermal growth factor receptor (EGFR), ErBb2 receptor on tumor cells, an internalising cellular receptor, LDL receptor, FGF2 receptor, ErbB2 receptor, transferrin receptor, PDGF receptor, VEGF receptor, PsmAr, an extracellular matrix protein, elastin, fibronectin, laminin, α1-antitrypsin, tissue factor protease inhibitor, PDK1, GSK1, Bad, caspase-9, Forkhead, an antigen of Helicobacter pylori, an antigen of Mycobacterium tuberculosis, and an antigen of influenza virus as well as any target disclosed in Annex 2 or Annex 3 hereto, whether in combination as set forth in the Annexes, in a different combination or individually.

Advantageously, according to the final aspect of the invention, preferably the dual-specific ligands exhibit the ability to neutralise in vitro or in cell based assays

Ligands according to any aspect of the present invention, as well as dAb monomers useful in constructing such ligands, may advantageously dissociate from their cognate target(s) with a K_{d} of 300 nM to 5 pM (ie, 3×10⁻⁷ to 5×10⁻¹²M), preferably 50 nM to 20 pM, or 5 nM to 200 pM or 1 nM to 100 pM, 1×10⁻⁷ M or less, 1×10⁻⁸ M or less, 1×10⁻⁹ M or less, 1×10⁻¹⁰ M or less, 1×10⁻¹¹ M or less; and/or a K_{off} rate constant of 5×10⁻¹ to 1×10⁻⁷ S⁻¹, preferably 1×10⁻² to 1×10⁻⁶ S⁻¹, or 5×10⁻³ to 5×10⁻⁵ S⁻¹, or 5×10⁻¹ S⁻¹ or less, or 1×10⁻² S⁻¹ or less, or 1×10⁻³ S⁻¹ or less, or 1×10⁻⁴ S⁻¹ or less, or 5×10⁻⁵ S⁻¹ or less, or 1×10⁻⁶ S⁻¹ or less as determined by surface plasmon resonance. The K_{d} rate constant is defined as K_{off}/Kₒₙ.

In particular the invention provides a dual-specific ligand wherein the affinity of binding to target with a K_{d} of 300 nM to 5 pM (ie, 3×10⁻⁷ to 5×10⁻¹²M), preferably 50 nM to 20 pM, more preferably 5 nM to 200 pM and most preferably 1 nM to 100 pM; expressed in an alternative manner, the K_{d} is 1×10⁻⁷ M or less, preferably 1×10⁻⁸ M or less, more preferably 1×10⁻⁹ M or less, advantageously 1×10⁻¹⁰ M or less and most preferably 1×10⁻¹¹ M or less; and/or a K_{off} rate constant of 5×10⁻¹ to 1×10⁻⁷ S⁻¹, preferably 5×10⁻² to 1×10⁻⁶ S⁻¹, more preferably 5×10⁻³ to 1×10⁻⁵ S⁻¹, for example 5×10⁻¹ S⁻¹, or less, preferably 1×10⁻² S⁻¹ or less, more preferably 1×10⁻³ S⁻¹ or less, advantageously 1×10⁻⁴ S⁻¹ or less, further advantageously 1×10⁻⁵ S⁻¹ or less, and most preferably 1×10⁻⁶ S^{-less, as determined by surface plasmon resonance.}

Preferably, the ligand neutralises TNFα in a standard L929 assay with an ND50 of 500 nM to 50 pM, preferably or 100 nM to 50 pM, advantageously 10 nM to 100 pM, more preferably 1 nM to 100 pM; for example 50 nM or less, preferably 5 nM or less, advantageously 500 pM or less, more preferably 200 pM or less and most preferably 100 pM or less.

Preferably, the ligand inhibits binding of TNF alpha to TNF alpha Receptor I (p55 receptor) with an IC50 of 500 nM to 50 pM, preferably 100 nM to 50 pM, more preferably 10 nM to 100 pM, advantageously 1 nM to 100 pM; for example 50 nM or less, preferably 5 nM or less, more preferably 500 pM or less, advantageously 200 pM or less, and most preferably 100 pM or less. Preferably, the TNFα is Human TNFα.

According to the above aspect of the invention dual-specific ligands preferably exhibit a binding affinity of at least 50 nM.

In a preferred aspect, the invention relates to a dual specific ligand which binds to a target ligand and a receptor for the target ligand. For example, the ligand may be TNFα and the target ligand receptor may be TNF Receptor 1. Advantageously, the dual specific ligand according to the invention is able to bind both the target ligand and the target ligand receptor simultaneously, i.e. is in an open configuration.

According to the present invention, advantageously a dual-specific ligand as described herein is a TAR1/TAR2 dual specific Fab, F(ab′)₂ or IgG as herein described and is specific for human TNF alpha and the human TNFR1 (p55 receptor). Preferably, each arm comprises a complementary VH/VL pair. More preferably, the VL of each pair is Vk. More preferably still the VK has TNF as target and the VH of each pair has the p55 receptor as a target. According to these Fab or IgG formats the dAbs advantageously bind their targets simultaneously, that is with no significant competition.

Most advantageously a TAR1/TAR2 IgG or Fab format dual-specific ligand is as described herein in the Examples.

Those skilled in the art will appreciate that the vectors/constructs provided in the Examples and used for the generation of TAR1/TAR2 dual-specific ligands and the dAbs comprising them represent a mere sample of suitable vectors/constructs for use according to the above aspect of the invention. Vectors/constructs suitable for use include the following:

(a) Eukaryotic leader-V_{H} or V_{L}-C_{H}1-hinge-C_{H}2-C_{H}3. In this embodiment the leader may be mammalian, for example a CD33 or IgG K leader or functional variant/fragment of these, or at least 80% homologous with any of these leaders.

According to the present invention there is also provided an expression vector, preferably yeast or mammalian in nature comprising a construct as described above in (a).

According to a further aspect still, there is provided a host, preferably mammalian cells such as Cos cells comprising a vector as described above.

In a final aspect of the invention there is provided a V_{H} dAb monomer designated TAR2h-10-27 dAb having the amino acid sequence given below (a) and which binds to the human TNF receptor 1 (p55 receptor):

EVQLLESGGGLVQPGGSLRLSCAASGFTFEWYWMGWVRQAPGKGLEWVSA
ISGSGGSTYYADSVKGRFTISRDNSKNTLYLQMNSLRAEDAAVYYCAKVK
LGGGPNFGYRGQGTLVTVSSAA

TAR2h-10-27 nucleic acid coding sequence

GAGGTGCAGCTGTTGGAGTCTGGGGGAGGCTTGGTACAGCCTGGGGGGTC
CCTGCGTCTCTCCTGTGCAGCCTCCGGATTCACCTTTGAGTGGTATTGGA
TGGGTTGGGTCCGCCAGGCTCCAGGGAAGGGTCTAGAGTGGGTCTCAGCT
ATCAGTGGTAGTGGTGGTAGCACATACTACGCAGACTCCGTGAAGGGCCG
GTTCACCATCTCCCGCGACAATTCCAAGAACACGCTGTATCTGCAAATGA
ACAGCCTGCGTGCCGAGGACGCCGCGGTATATTACTGTGCGAAAGTTAAG
TTGGGGGGGGGGCCTAATTTTGGCTACCGGGGCCAGGGAACCCTGGTCAC
CGTCTCGAGCGCGGCCGC

Advantageously, this dAb is comprised within a dual-specific ligand. Dual specific ligands include scFv, Fab and Ig molecules, and may be in open or closed conformations. Particularly preferred are dual specific Fab and IgG formats, comprising complementary TAR1-5-19 V_{κ} and TAR2h-10-27 V_{H} domains. Advantageously, the polypeptide is in an open conformation.

The present invention also describes methods of treating a TNF-α-elated inflammatory disorder in an individual suffering from such a disorder. The method comprises administering a therapeutically effective amount of a single domain antibody polypeptide construct, preferably a human single domain antibody construct, to such an individual, wherein the single domain antibody polypeptide construct binds human TNF-α, and whereby the TNF-α-related disorder is treated.

In one aspect, the inflammatory disorder is rheumatoid arthritis, and the method comprises the use of one or more single domain antibody polypeptide constructs, wherein one or more of the constructs antagonizes human TNFα's binding to a receptor. The present invention describes compositions comprising one or more single domain antibody polypeptide constructs that antagonize human TNFα's binding to a receptor, and dual specific ligands in which one specificity of the ligand is directed toward TNFα and a second specificity is directed to VEGF or HSA. The present invention further describes dual specific ligands in which one specificity of the ligand is directed toward VEGF and a second specificity is directed to HSA.

In one aspect, the invention encompasses a method of treating rheumatoid arthritis, the method comprising administering to an individual in need thereof a therapeutically effective amount of a composition comprising a single domain antibody polypeptide construct that antagonizes human TNFα's binding to a receptor, whereby the rheumatoid arthritis is treated.

In one embodiment, the composition prevents an increase in arthritic score when administered to a mouse of the Tg197 transgenic mouse model of arthritis.

In another embodiment, the administration of the composition to a Tg197 transgenic mouse comprises the following steps: a) administer weekly intraperitoneal injections of the composition to a heterozygous Tg197 transgenic mouse, b) weigh the mouse of step a) weekly, and c) score the mouse weekly for macrophenotypic signs of arthritis according to the following system: 0=no arthritis (normal appearance and flexion), 1=mild arthritis (joint distortion), 2=moderate arthritis (swelling, joint deformation), 3=heavy arthritis (severely impaired movement).

In another embodiment, the composition is administered to the mouse before the onset of arthritic symptoms is manifested. In another embodiment, the composition is first administered when the mouse is three weeks of age. In another embodiment, the composition is first administered when the mouse is six weeks of age

In another embodiment, the composition has an efficacy in the Tg197 transgenic mouse arthritis assay that is greater or equal, within the realm of statistical significance, to that of an equivalent dose (on a mg/kg basis) of an agent selected from the group consisting of Etanercept, Infliximab and D2E7.

In another embodiment, the composition has an efficacy in the Tg197 transgenic mouse arthritis assay, such that the treatment results in an arthritic score of 0 to 0.5. In another embodiment, the composition has an efficacy in the Tg197 transgenic mouse arthritis assay, such that the treatment results in an arthritic score of 0 to 1.0. In another embodiment, the composition has an efficacy in the Tg197 transgenic mouse arthritis assay, such that the treatment results in an arthritic score of 0 to 1.5. In another embodiment, the composition has an efficacy in the Tg197 transgenic mouse arthritis assay, such that the treatment results in an arthritic score of 0 to 2.0.

In another embodiment, the treating comprises inhibiting the progression of the rheumatoid arthritis. In another embodiment, the treating comprises preventing or delaying the onset of rheumatoid arthritis.

In another embodiment, the administering results in a statistically significant change in one or more indicia of RA. In another embodiment, the one or more indicia of

RA comprise one or more of erythrocyte sedimentation rate (ESR), Ritchie articular index and duration of morning stiffness, joint mobility, joint swelling, x ray imaging of one or more joints, and histopathological analysis of fixed sections of one or more joints.

In another embodiment, the one or more indicia of RA comprises a decrease in the macrophenotypic signs of arthritis in a Tg197 transgenic mouse, wherein the composition is administered to a Tg197 transgenic mouse, wherein the Tg197 transgenic mouse is scored for the macrophenotypic signs of arthritis, and wherein the macrophenotypic signs of arthritis are scored according to the following system: 0=no arthritis (normal appearance and flexion), 1=mild arthritis (joint distortion), 2=moderate arthritis (swelling, joint deformation), 3=heavy arthritis (severely impaired movement).

In another embodiment, the one or more indicia of RA comprises a decrease in the histopathological signs of arthritis in a Tg197 transgenic mouse, wherein the composition is administered to a Tg197 transgenic mouse, wherein the Tg197 transgenic mouse is scored for the histopathological signs of arthritis, and wherein the histopathological signs of arthritis are performed on a joint and scored using the following system: 0=no detectable pathology, 1=hyperplasia of the synovial membrane and presence of polymorphonuclear infiltrates, 2=pannus and fibrous tissue formation and focal subchondral bone erosion, 3=articular cartilage destruction and bone erosion, 4=extensive articular cartilage destruction and bone erosion.

In another embodiment, the single domain antibody polypeptide construct comprises a human single domain antibody polypeptide. In another embodiment, the human single domain antibody polypeptide binds TNFα. In another embodiment, the single domain antibody polypeptide construct binds human TNFα with a Kd of <100 nM. In another embodiment, the single domain antibody polypeptide construct binds human TNFα with a K_{d} in the range of 100 nM to 50 pM. In another embodiment, the single domain antibody polypeptide construct binds human TNFα with a K_{d} of 30 nM to 50 pM. In another embodiment, the single domain antibody polypeptide construct binds human TNFα with a K_{d} of 10 nM to 50 pM. In another embodiment, the single domain antibody polypeptide construct binds human TNFα with a K_{d} in the range of 1 nM to 50 pM.

In another embodiment, the single domain antibody polypeptide construct antagonizes human TNFα as measured in a standard L929 cytotoxicity cell assay.

The invention further encompasses a method of treating rheumatoid arthritis, the method comprising administering to an individual in need thereof a therapeutically effective amount of a composition comprising a single domain antibody polypeptide construct that antagonizes human TNFα's binding to a receptor, wherein the single domain antibody polypeptide construct inhibits the binding of human TNFα to a TNFα receptor, and whereby the rheumatoid arthritis is treated.

In one embodiment, the single domain antibody polypeptide construct specifically binds to human TNF-α which is bound to a cell surface receptor.

In another embodiment, the single domain antibody polypeptide construct has an in vivo tα-half life in the range of 15 minutes to 12 hours. In another embodiment, the single domain antibody polypeptide construct has an in vivo tβ-half life in the range of 1 to 6 hours. In another embodiment, the single domain antibody polypeptide construct has an in vivo tβ-half life in the range of 2 to 5 hours. In another embodiment, the single domain antibody polypeptide construct has an in vivo tβ-half life in the range of 3 to 4 hours. In another embodiment, the single domain antibody polypeptide construct has an in vivo tβ-half life in the range of 12 to 60 hours. In another embodiment, the single domain antibody polypeptide construct has an in vivo tβ-half life in the range of 12 to 48 hours. In another embodiment, the single domain antibody polypeptide construct has an in vivo tβ-half life in the range of 12 to 26 hours.

In another embodiment, the single domain antibody polypeptide construct has an in vivo AUC half-life value of 15 mg.min/ml to 150 mg.min/ml. In another embodiment, the single domain antibody polypeptide construct has an in vivo AUC half-life value of 15 mg.min/ml to 100 mg.min/ml. In another embodiment, the single domain antibody polypeptide construct has an in vivo AUC half-life value of 15 mg.min/ml to 75 mg.min/ml. In another embodiment, the single domain antibody polypeptide construct has an in vivo AUC half-life value of 15 mg.min/m1 to 50 mg.min/ml.

In another embodiment, the single domain antibody polypeptide construct is linked to a PEG molecule. In another embodiment, the PEG-linked single domain antibody polypeptide construct has a hydrodynamic size of at least 24 kDa, and wherein the total PEG size is from 20 to 60 kDa. In another embodiment, the PEG-linked single domain antibody polypeptide construct has a hydrodynamic size of at least 200 kDa and a total PEG size of from 20 to 60 kDa. In another embodiment, the PEGylated proteins of the invention may be linked, on average, to 1, 2, 3, 4, 5, 6, 7, 8, 9, 10, 12, 15, 17, 20, or more polyethylene glycol molecules.

In another embodiment, the antibody construct comprises two or more single immunoglobulin variable domain polypeptides that bind human TNFα. In another embodiment, the antibody construct comprises a homodimer of a single immunoglobulin variable domain polypeptide that binds human TNFα. In another embodiment, the antibody construct comprises a homotrimer of a single immunoglobulin variable domain polypeptide that binds human TNFα. In another embodiment, the antibody construct comprises a homotetramer of a single immunoglobulin variable domain polypeptide that binds human TNFα.

In another embodiment, the construct further comprises an antibody polypeptide specific for an antigen other than TNFα. In another embodiment, the antibody polypeptide specific for an antigen other than TNFα comprises a single domain antibody polypeptide. In another embodiment, the binding of the antigen other than TNFα by the antibody polypeptide specific for an antigen other than TNFα increases the in vivo half-life of the antibody polypeptide construct. In another embodiment, the antigen other than TNFα comprises a serum protein. In another embodiment, the serum protein is selected from the group consisting of fibrin, α-2 macroglobulin, serum albumin, fibrinogen A, fibrinogen, serum amyloid protein A, heptaglobin, protein, ubiquitin, uteroglobulin and β- 2-microglobulin. In another embodiment, the antigen other than TNFα comprises HSA.

In another embodiment, the treating further comprises administration of at least one additional therapeutic agent.

In another embodiment, the single domain antibody polypeptide construct comprises the amino acid sequence of CDR3 of an antibody polypeptide selected from the group consisting of clones TAR1-2m-9, TAR1-2m-30,TAR1-2m-1,TAR1-2m-2, TAR1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-5, TAR1-5-36, TAR1-5-464, TAR1-5-463, TAR1-5-460, TAR1-5-461, TAR1-5-479, TAR1-5-477, TAR1-5-478, TAR1-5-476, TAR1-5-490, TAR1h-1, TAR1h-2, TAR1h-3, TAR1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR1-100-60, TAR1-100-62, TAR1-100-64, TAR1-100-65, TAR1-100-75, TAR1-100-76, TAR1-100-77, TAR1-100-78, TAR1-100-80, TAR1-100-82, TAR1-100-83, TAR1-100-84, TAR1-100-89, TAR1-100-90, TAR1-100-91, TAR1-100-92, TAR1-100-93, TAR1-100-94, TAR1-100-95, TAR1-100-96, TAR1-100-97, TAR1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19.

In another embodiment, the single domain antibody polypeptide construct comprises the amino acid sequence of an antibody polypeptide selected from the group consisting of clones TAR1-2m-9, TAR1-2m-30,TAR1-2m-1,TAR1-2m-2, TAR1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-35, TAR1-5-36, TAR1-5-464, TAR1-5-463, TAR1-5-460, TAR1-5-461, TAR1-5-479, TAR1-5-477, TAR1-5-478, TAR1-5-476, TAR1-5-490, TAR1h-1, TAR1h-2, TAR1h-3, TAR1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR1-100-60, TAR1-100-62, TAR1-100-64, TAR1-100-65, TAR1-100-75, TAR1-100-76, TAR1-100-77, TAR1-100-78, TAR1-100-80, TAR1-100-82, TAR1-100-83, TAR1-100-84, TAR1-100-89, TAR1-100-90, TAR1-100-91, TAR1-100-92, TAR1-100-93, TAR1-100-94, TAR1-100-95, TAR1-100-96, TAR1-100-97, TAR1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19 or an amino acid sequence at least 85% identical thereto.

In another embodiment, the single domain antibody polypeptide construct comprises the amino acid sequence of an antibody polypeptide selected from the group consisting of clones TAR1-2m-9, TAR1-2m-30,TAR1-2m-1,TAR1-2m-2, TAR1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-35, TAR1-5-36, TAR1-5-464, TAR1-5-463, TAR1-5-460, TAR1-5-461, TAR1-5-479, TAR1-5-477, TAR1-5-478, TAR1-5-476, TAR1-5-490, TAR1h-1, TAR1h-2, TAR1h-3, TAR1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR1-100-60, TAR1-100-62, TAR1-100-64, TAR1-100-65, TAR1-100-75, TAR1-100-76, TAR1-100-77, TAR1-100-78, TAR1-100-80, TAR1-100-82, TAR1-100-83, TAR1-100-84, TAR1-100-89, TAR1-100-90, TAR1-100-91, TAR1-100-92, TAR1-100-93, TAR1-100-94, TAR 1-100-95, TAR1-100-96, TAR1-100-97, TAR1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19 or an amino acid sequence at least 90% identical thereto.

In another embodiment, the single domain antibody polypeptide construct comprises the amino acid sequence of an antibody polypeptide selected from the group consisting of clones TAR1-2m-9, TAR1-2m-30,TAR1-2m-1,TAR1-2m-2, TAR1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-35, TAR1-5-36, TAR1-5-464, TAR1-5-463, TAR1-5-460, TAR1-5-461, TAR1-5-479, TAR1-5-477, TAR1-5-478, TAR1-5-476, TAR1-5-490, TAR1h-1, TAR1h-2, TAR1h-3, TAR1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR1-100-60, TAR1-100-62, TAR1-100-64, TAR1-100-65, TAR1-100-75, TAR1-100-76, TAR1-100-77, TAR1-100-78, TAR1-100-80, TAR1-100-82, TAR1-100-83, TAR1-100-84, TAR1-100-89, TAR1-100-90, TAR1-100-91, TAR1-100-92, TAR1-100-93, TAR1-100-94, TAR1-100-95, TAR1-100-96, TAR1-100-97, TAR1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19 or an amino acid sequence at least 92% identical thereto.

In another embodiment, the single domain antibody polypeptide construct comprises the amino acid sequence of an antibody polypeptide selected from the group consisting of clones TAR1-2m-9, TAR1-2m-30,TAR1-2m-1,TAR1-2m-2, TAR1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-35, TAR1-5-36, TAR1-5-464, TAR1-5-463, TAR1-5-460, TAR1-5-461, TAR1-5-479, TAR1-5-477, TAR1-5-478, TAR1-5-476, TAR1-5-490, TAR1h-1, TAR1h-2, TAR1h-3, TAR1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR1-100-60, TAR 1-100-62, TAR 1-100-64, TAR1-100-65, TAR 1-100-75, TAR1-100-76, TAR1-100-77, TAR1-100-78, TAR1-100-80, TAR1-100-82, TAR1-100-83, TAR1-100-84, TAR1-100-89, TAR1-100-90, TAR1-100-91, TAR1-100-92, TAR1-100-93, TAR1-100-94, TAR1-100-95, TAR1-100-96, TAR1-100-97, TAR1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19 or an amino acid sequence at least 94% identical thereto.

In another embodiment, the single domain antibody polypeptide construct comprises the amino acid sequence of an antibody polypeptide selected from the group consisting of clones TAR1-2m-9, TAR1-2m-30,TAR1-2m-1,TAR1-2m-2, TAR1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-35, TAR1-5-36, TAR1-5-464, TAR1-5-463, TAR1-5-460, TAR1-5-461, TAR1-5-479, TAR1-5-477, TAR1-5-478, TAR1-5-476, TAR1-5-490, TAR1h-1, TAR1h-2, TAR1h-3, TAR1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR1-100-60, TAR 1-100-62, TAR1-100-64, TAR1-100-65, TAR1-100-75, TAR1-100-76, TAR1-100-77, TAR 1-100-78, TAR 1-100-80, TAR 1-100-82, TAR 1-100-83, TAR1-100-84, TAR1-100-89, TAR1-100-90, TAR1-100-91, TAR1-100-92, TAR1-100-93, TAR1-100-94, TAR1-100-95, TAR1-100-96, TAR1-100-97, TAR1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19 or an amino acid sequence at least 96% identical thereto.

In another embodiment, the single domain antibody polypeptide construct comprises the amino acid sequence of an antibody polypeptide selected from the group consisting of clones TAR1-2m-9, TAR1-2m-30,TAR1-2m-1,TAR1-2m-2, TAR1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-35, TAR1-5-36, TAR1-5-464, TAR1-5-463, TAR1-5-460, TAR1-5-461, TAR1-5-479, TAR1-5-477, TAR1-5-478, TAR1-5-476, TAR1-5-490, TAR1h-1, TAR1h-2, TAR1h-3, TAR1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR 1-100-60, TAR 1-100-62, TAR 1-100-64, TAR1-100-65, TAR1-100-75, TAR 1-100-76, TAR1-100-77, TAR1-100-78, TAR1-100-80, TAR1-100-82, TAR1-100-83, TAR1-100-84, TAR1-100-89, TAR1-100-90, TAR1-100-91, TAR1-100-92, TAR1-100-93, TAR1-100-94, TAR1-100-95, TAR1-100-96, TAR1-100-97, TAR1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19 or an amino acid sequence at least 98% identical thereto.

In another embodiment, the single domain antibody polypeptide construct comprises the amino acid sequence of an antibody polypeptide selected from the group consisting of clones TAR1-2m-9, TAR1-2m-30,TAR1-2m-1,TAR1-2m-2, TAR1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-35, TAR1-5-36, TAR1-5-464, TAR1-5-463, TAR1-5-460, TAR1-5-461, TAR1-5-479, TAR1-5-477, TAR1-5-478, TAR1-5-476, TAR1-5-490, TAR1h-1, TAR1h-2, TAR1h-3, TAR1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR 1-100-60, TAR1-100-62, TAR1-100-64, TAR1-100-65, TAR1-100-75, TAR1-100-76, TAR 1-100-77, TAR 1-100-78, TAR 1-100-80, TAR 1-100-82, TAR 1-100-83, TAR1-100-84, TAR1-100-89, TAR1-100-90, TAR1-100-91, TAR1-100-92, TAR1-100-93, TAR1-100-94, TAR1-100-95, TAR1-100-96, TAR1-100-97, TAR1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19 or an amino acid sequence at least 99% identical thereto.

The invention further encompasses a method of treating rheumatoid arthritis, the method comprising administering to an individual in need thereof, a therapeutically effective amount of a composition comprising a single domain antibody polypeptide construct that antagonizes human TNFα's binding to a receptor, wherein the composition prevents an increase in arthritic score when administered to a mouse of the Tg197 transgenic mouse model of arthritis, wherein the single domain antibody polypeptide construct binds human TNFα with a Kd of <100 nM, wherein the single domain antibody polypeptide construct neutralizes human TNFα as measured in a standard L929 cell assay, and wherein the rheumatoid arthritis is treated.

The invention further encompasses a composition comprising a single domain antibody polypeptide construct that antagonizes human TNFα's binding to a receptor, and that prevents an increase in arthritic score when administered to a mouse of the Tg197 transgenic mouse model of arthritis, wherein the single domain antibody polypeptide construct neutralizes human TNFα as measured in a standard L929 cell assay.

The invention further encompasses a composition comprising a single domain antibody polypeptide construct that antagonizes human TNFα's binding to a receptor, that prevents an increase in arthritic score when administered to a mouse of the Tg197 transgenic mouse model of arthritis, wherein the single domain antibody polypeptide construct inhibits the progression of the rheumatoid arthritis.

The invention further encompasses a composition comprising a single domain antibody polypeptide construct that antagonizes human TNFα's binding to a receptor, that prevents an increase in arthritic score when administered to a mouse of the Tg197 transgenic mouse model of arthritis, wherein the single domain antibody polypeptide construct binds human TNFα with a Kd of <100 nM.

The invention further encompasses a composition comprising a single domain antibody polypeptide construct that antagonizes human TNFα's binding to a receptor, that prevents an increase in arthritic score when administered to a mouse of the Tg197 transgenic mouse model of arthritis, wherein the single domain antibody polypeptide construct neutralizes human TNFα as measured in a standard L929 cell assay, wherein the single domain antibody polypeptide construct inhibits the progression of the rheumatoid arthritis, wherein the single domain antibody polypeptide construct binds human TNFα with a Kd of <100 nM.

In a further embodiment of the preceding 3 embodiments, the single domain antibody polypeptide construct comprises the amino acid sequence of CDR3 of an antibody polypeptide selected from the group consisting of clones TAR1-2m-9, TAR1-2m-30,TAR1-2m-1,TAR1-2m-2, TAR1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-35, TAR1-5-36, TAR1-5-464, TAR1-5-463, TAR1-5-460, TAR1-5-461, TAR1-5-479, TAR1-5-477, TAR1-5-478, TAR1-5-476, TAR1-5-490, TAR1h-1, TAR1h-2, TAR1h-3, TAR1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR1-100-60, TAR1-100-62, TAR1-100-64, TAR1-100-65, TAR1-100-75, TAR1-100-76, TAR1-100-77, TAR1-100-78, TAR1-100-80, TAR1-100-82, TAR1-100-83, TAR1-100-84, TAR1-100-89, TAR1-100-90, TAR1-100-91, TAR1-100-92, TAR1-100-93, TAR1-100-94, TAR1-100-95, TAR1-100-96, TAR1-100-97, TAR1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19.

In a further embodiment the single domain antibody polypeptide construct comprises the amino acid sequence of an antibody polypeptide selected from the group consisting of clones TAR1-2m-9, TAR1-2m-30,TAR1-2m-1,TAR1-2m-2, TAR1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-35, TAR1-5-36, TAR1-5-464, TAR1-5-463, TAR1-5-460, TAR1-5-461, TAR1-5-479, TAR1-5-477, TAR1-5-478, TAR 1-5-476, TAR 1-5-490, TAR1h-1, TAR1h-2, TAR1h-3, TAR 1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR1-100-60, TAR1-100-62, TAR1-100-64, TAR1-100-65, TAR1-100-75, TAR1-100-76, TAR1-100-77, TAR1-100-78, TAR1-100-80, TAR1-100-82, TAR1-100-83, TAR1-100-84, TAR1-100-89, TAR1-100-90, TAR1-100-91, TAR1-100-92, TAR1-100-93, TAR1-100-94, TAR1-100-95, TAR1-100-96, TAR1-100-97, TAR 1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19 or an amino acid sequence at least 85% identical thereto.

In a further embodiment the single domain antibody polypeptide construct comprises the amino acid sequence of an antibody polypeptide selected from the group consisting of clones TAR I -2m-9, TAR I -2m-30,TAR1-2m-1,TAR1-2m-2, TAR 1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-35, TAR1-5-36, TAR1-5-464, TAR1-5-463, TAR1-5-460, TAR1-5-461, TAR1-5-479, TAR1-5-477, TAR1-5-478, TAR1-5-476, TAR1-5-490, TAR1h-1, TAR1h-2, TAR1h-3, TAR1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR1-100-60, TAR1-100-62, TAR1-100-64, TAR1-100-65, TAR1-100-75, TAR1-100-76, TAR1-100-77, TAR1-100-78, TAR1-100-80, TAR1-100-82, TAR1-100-83, TAR1-100-84, TAR1-100-89, TAR1-100-90, TAR1-100-91, TAR1-100-92, TAR1-100-93, TAR1-100-94, TAR1-100-95, TAR1-100-96, TAR1-100-97, TAR1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19 or an amino acid sequence at least 90% identical thereto.

In a further embodiment the single domain antibody polypeptide construct comprises the amino acid sequence of an antibody polypeptide selected from the group consisting of clones TAR1-2m-9, TAR1-2m-30,TAR1-2m-1,TAR1-2m-2, TAR1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-35, TAR1-5-36, TAR1-5-464, TAR1-5-463, TAR1-5-460, TAR1-5-461, TAR1-5-479, TAR1-5-477, TAR1-5-478, TAR1-5-476, TAR1-5-490, TAR1h-1, TAR1h-2, TAR1h-3, TAR1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR1-100-60, TAR1-100-62, TAR1-100-64, TAR1-100-65, TAR1-100-75, TAR1-100-76, TAR 1-100-77, TAR 1-100-78, TAR 1-100-80, TAR1-100-82, TAR 1-100-83, TAR1-100-84, TAR1-100-89, TAR1-100-90, TAR1-100-91, TAR1-100-92, TAR1-100-93, TAR1-100-94, TAR1-100-95, TAR1-100-96, TAR1-100-97, TAR1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19 or an amino acid sequence at least 92% identical thereto.

In a further embodiment the single domain antibody polypeptide construct comprises the amino acid sequence of an antibody polypeptide selected from the group consisting of clones TAR1-2m-9, TAR1-2m-30,TAR1-2m-1,TAR1-2m-2, TAR1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-35, TAR1-5-36, TAR1-5-464, TAR1-5-463, TAR1-5-460, TAR1-5-461, TAR1-5-479, TAR1-5-477, TAR1-5-478, TAR1-5-476, TAR1-5-490, TAR1h-1, TAR1h-2, TAR1h-3, TAR1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR1-100-60, TAR1-100-62, TAR1-100-64, TAR1-100-65, TAR1-100-75, TAR1-100-76, TAR1-100-77, TAR1-100-78, TAR1-100-80, TAR1-100-82, TAR1-100-83, TAR1-100-84, TAR1-100-89, TAR1-100-90, TAR1-100-91, TAR1-100-92, TAR1-100-93, TAR1-100-94, TAR1-100-95, TAR1-100-96, TAR1-100-97, TAR1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19 or an amino acid sequence at least 94% identical thereto.

In a further embodiment the single domain antibody polypeptide construct comprises the amino acid sequence of an antibody polypeptide selected from the group consisting of clones TAR1-2m-9, TAR1-2m-30,TAR1-2m-1,TAR1-2m-2, TAR1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-35, TAR1-5-36, TAR1-5-464, TAR1-5-463, TAR1-5-460, TAR1-5-461, TAR1-5-479, TAR1-5-477, TAR1-5-478, TAR1-5-476, TAR1-5-490, TAR1h-1, TAR1h-2, TAR1h-3, TAR1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR1-100-60, TAR1-100-62, TAR1-100-64, TAR1-100-65, TAR1-100-75, TAR1-100-76, TAR1-100-77, TAR1-100-78, TAR1-100-80, TAR1-100-82, TAR1-100-83, TAR1-100-84, TAR1-100-89, TAR1-100-90, TAR1-100-91, TAR1-100-92, TAR1-100-93, TAR1-100-94, TAR1-100-95, TAR1-100-96, TAR1-100-97, TAR1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19 or an amino acid sequence at least 96% identical thereto.

In a further embodiment the single domain antibody polypeptide construct comprises the amino acid sequence of an antibody polypeptide selected from the group consisting of clones TAR1-2m-9, TAR1-2m-30,TAR1-2m-1,TAR1-2m-2, TAR1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-35, TAR1-5-36, TAR1-5-464, TAR1-5-463, TAR1-5-460, TAR1-5-461, TAR1-5-479, TAR1-5-477, TAR1-5-478, TAR1-5-476, TAR1-5-490, TAR1h-1, TAR1h-2, TAR1h-3, TAR1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR1-100-60, TAR1-100-62, TAR1-100-64, TAR1-100-65, TAR1-100-75, TAR1-100-76, TAR1-100-77, TAR1-100-78, TAR1-100-80, TAR1-100-82, TAR1-100-83, TAR1-100-84, TAR1-100-89, TAR1-100-90, TAR1-100-91, TAR1-100-92, TAR1-100-93, TAR1-100-94, TAR1-100-95, TAR1-100-96, TAR1-100-97, TAR1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19 or an amino acid sequence at least 98% identical thereto.

In a further embodiment the single domain antibody polypeptide construct comprises the amino acid sequence of an antibody polypeptide selected from the group consisting of clones TAR1-2m-9, TAR1-2m-30,TAR1-2m-1,TAR1-2m-2, TAR1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-35, TAR1-5-36, TAR1-5-464, TAR1-5-463, TAR1-5-460, TAR1-5-461, TAR1-5-479, TAR1-5-477, TAR1-5-478, TAR1-5-476, TAR1-5-490, TAR1h-1, TAR1h-2, TAR1h-3, TAR1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR1-100-60, TAR1-100-62, TAR1-100-64, TAR1-100-65, TAR1-100-75, TAR1-100-76, TAR1-100-77, TAR1-100-78, TAR1-100-80, TAR1-100-82, TAR1-100-83, TAR1-100-84, TAR1-100-89, TAR1-100-90, TAR1-100-91, TAR1-100-92, TAR1-100-93, TAR1-100-94, TAR1-100-95, TAR1-100-96, TAR1-100-97, TAR1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19 or an amino acid sequence at least 98% identical thereto.

In a further embodiment the single domain antibody polypeptide construct comprises the amino acid sequence of an antibody polypeptide selected from the group consisting of clones TAR1-2m-9, TAR1-2m-30,TAR1-2m-1,TAR1-2m-2, TAR1-5, TAR1-27, TAR1-261, TAR1-398, TAR1-701,TAR1-5-2, TAR1-5-3, TAR1-5-4, TAR1-5-7, TAR1-5-8, TAR1-5-10, TAR1-5-11, TAR1-5-12, TAR1-5-13, TAR1-5-19, TAR1-5-20, TAR1-5-21, TAR1-5-22, TAR1-5-23, TAR1-5-24, TAR1-5-25, TAR1-5-26, TAR1-5-27, TAR1-5-28, TAR1-5-29, TAR1-5-34, TAR1-5-35, TAR1-5-36, TAR1-5-464, TAR1-5-463, TAR1-5-460, TAR1-5-461, TAR1-5-479, TAR1-5-477, TAR1-5-478, TAR1-5-476, TAR1-5-490, TAR1h-1, TAR1h-2, TAR1h-3, TAR1-100-29, TAR1-100-35, TAR1-100-43, TAR1-100-47, TAR1-100-52, TAR1-109, TAR1-100, TAR1-100-34, TAR1-100-36, TAR1-100-38, TAR1-100-39, TAR1-100-40, TAR1-100-41, TAR1-100-45, TAR1-100-60, TAR1-100-62, TAR1-100-64, TAR 1-100-65, TAR1-100-75, TAR1-100-76, TAR1-100-77, TAR1-100-78, TAR1-100-80, TAR1-100-82, TAR1-100-83, TAR1-100-84, TAR1-100-89, TAR1-100-90, TAR1-100-91, TAR1-100-92, TAR1-100-93, TAR1-100-94, TAR1-100-95, TAR1-100-96, TAR1-100-97, TAR1-100-98, TAR1-100-99, TAR1-100-100, TAR1-100-101, TAR1-100-102, TAR1-100-103, TAR1-100-105, TAR1-100-106, TAR1-100-107, TAR1-100-108, TAR1-100-109, TAR1-100-110, TAR1-100-111, TAR1-100-112, TAR1-100-113 and TAR1-5-19 or an amino acid sequence at least 99% identical thereto.

The invention further encompasses a method of treating rheumatoid arthritis, the method comprising administering to an individual in need thereof a therapeutically effective amount of a composition comprising a single domain antibody polypeptide construct that antagonizes human VEGF's binding to a receptor, whereby the rheumatoid arthritis is treated.

In one embodiment the composition prevents an increase in arthritic score when administered to a mouse from a collagen induced arthritis (CIA) mouse model. Immunization of DBA/1 mice with murine type II collagen induces a chronic relapsing polyarthritis that provides a strong model for human autoimmune arthritis. The model is described, for example, by Courtenay et al., 1980, Nature 282 :666-668, Kato et al., 1996, Ann. Rheum. Dis. 55:535-539 and Myers et al., 1997, Life Sci. 61 :1861-1878, each of which is incorporated herein by reference.

In one embodiment the administration of the composition to the mouse comprises the following steps: a) administer weekly intraperitoneal injections of the composition to the CIA mouse, b) weigh the mouse of step a) weekly, and c) score the mouse weekly for macrophenotypic signs of arthritis according to the following system: 0=no arthritis (normal appearance and flexion), 1=mild arthritis (joint distortion), 2=moderate arthritis (swelling, joint deformation), 3=heavy arthritis (severely impaired movement).

In one embodiment the treating comprises inhibiting the progression of the rheumatoid arthritis.

In one embodiment the treating comprises preventing or delaying the onset of rheumatoid arthritis.

In one embodiment the administering results in a statistically significant change in one or more indicia of RA. The change is preferably by at least 10% or more.

In one embodiment the one or more indicia of RA comprise one or more of erythrocyte sedimentation rate (ESR), Ritchie articular index (described in Ritchie et al., b 1968, Q. J. Med. 37: 393-406) and duration of morning stiffness, joint mobility, joint swelling, analysis by x ray imaging of one or more joints, and histopathological indications by analysis of fixed sections of one or more joints. Disease activity and change effected with treatment can also be evaluated using the disease activity score (DAS) and/or the chronic arthritis systemic index (CASI), see Carotti et al., 2002, Ann. Rheum. Dis. 61:877-882, and Salaffi et al., 2000, Rheumatology 39: 90-96.

In one embodiment the one or more indicia of RA comprises a decrease in the macrophenotypic signs of arthritis in a mouse from a collagen induced arthritis mouse model, wherein the composition is administered to the mouse, wherein the mouse is scored for the macrophenotypic signs of arthritis, and wherein the macrophenotypic signs of arthritis are scored according to the following system: 0=no arthritis (normal appearance and flexion), 1=mild arthritis (joint distortion), 2=moderate arthritis (swelling, joint deformation), 3=heavy arthritis (severely impaired movement).

In one embodiment the one or more indicia of RA comprises a decrease in the histopathological signs of arthritis in a mouse from a collagen induced arthritis mouse model, wherein the composition is administered to the mouse, wherein the mouse is scored for the histopathological signs of arthritis, and wherein the histopathological signs of arthritis are performed on a joint and scored using the following system: 0=no detectable pathology, 1=hyperplasia of the synovial membrane and presence of polymorphonuclear infiltrates, 2=pannus and fibrous tissue formation and focal subchondral bone erosion, 3=articular cartilage destruction and bone erosion, 4=extensive articular cartilage destruction and bone erosion.

In one embodiment the single domain antibody polypeptide construct comprises a human single domain antibody polypeptide.

In one embodiment the human single domain antibody polypeptide binds VEGF.

In one embodiment the single domain antibody polypeptide construct binds human VEGF with a Kd of <100 nM.

In one embodiment the single domain antibody polypeptide construct binds human VEGF with a Kd in the range of 100 nM to 50 pM.

In one embodiment the single domain antibody polypeptide construct binds human VEGF with a Kd of 30 nM to 50 pM.

In one embodiment the single domain antibody polypeptide construct binds human VEGF with a Kd of 10 nM to 50 pM.

In one embodiment the single domain antibody polypeptide construct binds human VEGF with a Kd in the range of 1 nm to 50 pM.

In one embodiment the single domain antibody polypeptide construct neutralizes human VEGF as measured in a VEGF receptor 1 assay or a VEGF receptor 2 assay.

The invention further encompasses a method of treating rheumatoid arthritis, the method comprising administering to an individual in need thereof a therapeutically effective amount of a composition comprising a single domain antibody polypeptide construct that antagonizes human VEGF's's binding to a receptor, wherein the single domain antibody polypeptide construct inhibits the binding of human VEGF to a VEGF receptor, and whereby the rheumatoid arthritis is treated.

In one embodiment the single domain antibody polypeptide construct specifically binds to human VEGF which is bound to a cell surface receptor.

In one embodiment the single domain antibody polypeptide construct is linked to a PEG molecule.

In one embodiment the PEG-linked single domain antibody polypeptide construct has a hydrodynamic size of at least 24 kDa, and wherein the total PEG size is from 20 to 60 kDa.

In one embodiment the PEG-linked single domain antibody polypeptide construct has a hydrodynamic size of at least 200 kDa and a total PEG size of from 20 to 60 kDa.

In one embodiment the PEGylated proteins of the invention may be linked, on average, to 1, 2, 3, 4, 5, 6, 7, 8, 9, 10, 12, 15, 17, 20, or more polyethylene glycol molecules.

In one embodiment the antibody construct comprises two or more single immunoglobulin variable domain polypeptides that bind human VEGF.

In one embodiment the antibody construct comprises a homodimer of a single immunoglobulin variable domain polypeptide that binds human VEGF.

In one embodiment the antibody construct comprises a homotrimer of a single immunoglobulin variable domain polypeptide that binds human VEGF.

In one embodiment the antibody construct comprises a homotetramer of a single immunoglobulin variable domain polypeptide that binds human VEGF.

In one embodiment the construct further comprises an antibody polypeptide specific for an antigen other than VEGF.

In one embodiment the antibody polypeptide specific for an antigen other than VEGF comprises a single domain antibody polypeptide.

In one embodiment the binding of the antigen other than VEGF by the antibody polypeptide specific for an antigen other than VEGF increases the in vivo half-life of the antibody polypeptide construct.

In one embodiment the antigen other than VEGF comprises a serum protein.

In one embodiment the serum protein is selected from the group consisting of fibrin, α-2 macroglobulin, serum albumin, fibrinogen A, fibrinogen, serum amyloid protein A, heptaglobin, protein, ubiquitin, uteroglobulin and β-2-microglobulin.

In one embodiment the antigen other than VEGF comprises HSA.

In one embodiment, the single domain antibody polypeptide construct has an in vivo tβ-half life in the range of 15 minutes to 12 hours. In another embodiment, the single domain antibody polypeptide construct has an in vivo tβ-half life in the range of 1 to 6 hours.

In another embodiment, the single domain antibody polypeptide construct has an in vivo tβ-half life in the range of 2 to 5 hours. In another embodiment, the single domain antibody polypeptide construct has an in vivo tβ-half life in the range of 3 to 4 hours.

In another embodiment, the single domain antibody polypeptide construct has an in vivo tβ-half life in the range of 12 to 60 hours. In another embodiment, the single domain antibody polypeptide construct has an in vivo tβ-half life in the range of 12 to 48 hours.

In another embodiment, the single domain antibody polypeptide construct has an in vivo tβ-half life in the range of 12 to 26 hours. In another embodiment, single domain antibody polypeptide construct has an in vivo AUC half-life value of 15 mg.min/ml to 150 mg.min/ml. In another embodiment, the single domain antibody polypeptide construct has an in vivo AUC half-life value of 15 mg.min/ml to 100 mg.min/ml. In another embodiment, the single domain antibody polypeptide construct has an in vivo AUC half-life value of 15 mg.min/ml to 75 mg.min/ml. In another embodiment, the single domain antibody polypeptide construct has an in vivo AUC half-life value of 15 mg.min/ml to 50 mg.min/ml.

In one embodiment the treating further comprises administration of at least one additional therapeutic agent.

In one embodiment the therapeutic agent is selected from the group consisting of Etanercept, inflixmab and D2E7.

In one embodiment the therapeutic agent is selected from the group consisting of Corticosteroids, Proteolytic enzymes, non-steroidal anti-inflammatory drugs (NTHES), Acetylsalicylic acid, pyrazolones, fenamate, diflunisal, acetic acid derivatives, propionic acid derivatives, oxicams, mefenamic acid, Ponstel, meclofenamate, Meclomen, phenylbutazone, Butazolidin, diflunisal, Dolobid, diclofenac, Voltaren, indomethacin, Indocin, sulindac, Clinoril, etodolac, Lodine, ketorolac, Toradol, nabumetone, Relafen, tolmetin, Tolectin, ibuprofen, Motrin, fenoprofen, Nalfon, flurbiprofen, Anthe, carprofen, Rimadyl, ketoprofen, Orudis, naproxen, Anaprox, Naprosyn, piroxicam and Feldene.

In one embodiment the single domain antibody polypeptide construct comprises the amino acid sequence of CDR3 of an antibody polypeptide selected from the group consisting of clones TAR15-1, TAR15-3, TAR15-4, TAR15-9, TAR15-10, TAR15-11, TAR15-12, TAR15-13, TAR15-14, TAR15-15, TAR15-16, TAR15-17, TAR15-18, TAR15-19, TAR15-20, TAR 15-22, TAR15-5, TAR15-6, TAR15-7, TAR15-8, TAR15-23, TAR15-24, TAR15-25, TAR15-26, TAR15-27, TAR15-29, and TAR15-30.

In one embodiment the single domain antibody polypeptide construct comprises the amino acid sequence of an antibody polypeptide selected from the group consisting of clones TAR15-1, TAR15-3, TAR15-4, TAR15-9, TAR15-10, TAR15-11, TAR15-12, TAR15-13, TAR15-14, TAR15-15, TAR15-16, TAR15-17, TAR15-18, TAR15-19, TAR15-20, TAR 15-22, TAR15-5, TAR15-6, TAR15-7, TAR15-8, TAR15-23, TAR15-24, TAR15-25, TAR15-26, TAR15-27, TAR15-29, and TAR15-30 or an amino acid sequence at least 85% identical thereto.

In one embodiment the single domain antibody polypeptide construct comprises the amino acid sequence of an antibody polypeptide selected from the group consisting of clones TAR15-1, TAR15-3, TAR15-4, TAR15-9, TAR15-10, TAR15-11, TAR15-12, TAR15-13, TAR15-14, TAR15-15, TAR15-16, TAR15-17, TAR15-18, TAR15-19, TAR15-20, TAR 15-22, TAR15-5, TAR15-6, TAR15-7, TAR15-8, TAR15-23, TAR15-24, TAR15-25, TAR15-26, TAR15-27, TAR15-29, and TAR15-30 or an amino acid sequence at least 90% identical thereto.

In one embodiment the single domain antibody polypeptide construct comprises the amino acid sequence of an antibody polypeptide selected from the group consisting of clones TAR15-1, TAR15-3, TAR15-4, TAR15-9, TAR15-10, TAR15-11, TAR15-12, TAR15-13, TAR15-14, TAR15-15, TAR15-16, TAR15-17, TAR15-18, TAR15-19, TAR15-20, TAR 15-22, TAR15-5, TAR15-6, TAR15-7, TAR15-8, TAR15-23, TAR15-24, TAR15-25, TAR15-26, TAR15-27, TAR15-29, and TAR15-30 or an amino acid sequence at least 92% identical thereto.

In one embodiment the single domain antibody polypeptide construct comprises the amino acid sequence of an antibody polypeptide selected from the group consisting of clones TAR15-1, TAR15-3, TAR15-4, TAR15-9, TAR15-10, TAR15-11, TAR15-12, TAR15-13, TAR15-14, TAR15-15, TAR15-16, TAR15-17, TAR15-18, TAR15-19, TAR15-20, TAR15-22, TAR15-5, TAR15-6, TAR15-7, TAR15-8, TAR15-23, TAR15-24, TAR15-25, TAR15-26, TAR15-27, TAR15-29, and TAR15-30 or an amino acid sequence at least 94% identical thereto.

In one embodiment the single domain antibody polypeptide construct comprises the amino acid sequence of an antibody polypeptide selected from the group consisting of clones TAR15-1, TAR15-3, TAR15-4, TAR15-9, TAR15-10, TAR15-11, TAR15-12, TAR15-13, TAR15-14, TAR15-15, TAR15-16, TAR15-17, TAR15-18, TAR15-19, TAR15-20, TAR15-22, TAR15-5, TAR15-6, TAR15-7, TAR15-8, TAR15-23, TAR15-24, TAR15-25, TAR15-26, TAR15-27, TAR15-29, and TAR15-30 or an amino acid sequence at least 96% identical thereto.

In one embodiment the single domain antibody polypeptide construct comprises the amino acid sequence of an antibody polypeptide selected from the group consisting of clones TAR15-1, TAR15-3, TAR15-4, TAR15-9, TAR15-10, TAR15-11, TAR15-12, TAR15-13, TAR15-14, TAR15-15, TAR15-16, TAR15-17, TAR15-18, TAR15-19, TAR15-20, TAR 15-22, TAR15-5, TAR15-6, TAR15-7, TAR15-8, TAR15-23, TAR15-24, TAR15-25, TAR15-26, TAR15-27, TAR15-29, and TAR15-30 or an amino acid sequence at least 98% identical thereto.

In one embodiment the single domain antibody polypeptide construct comprises the amino acid sequence of an antibody polypeptide selected from the group consisting of clones TAR15-1, TAR15-3, TAR15-4, TAR15-9, TAR15-10, TAR15-11, TAR15-12, TAR15-13, TAR15-14, TAR15-15, TAR15-16, TAR15-17, TAR15-18, TAR15-19, TAR15-20, TAR15-22, TAR15-5, TAR15-6, TAR15-7, TAR15-8, TAR15-23, TAR15-24, TAR15-25, TAR15-26, TAR15-27, TAR15-29, and TAR15-30 or an amino acid sequence at least 99% identical thereto.

The invention further encompasses a composition comprising a single domain antibody polypeptide construct that antagonizes human VEGF binding to a receptor, wherein the single domain antibody polypeptide construct comprises a CDR3 sequence selected from the group consisting of TAR15-1, TAR15-3, TAR15-4, TAR15-9, TAR15-10, TAR15-11, TAR15-12, TAR15-13, TAR15-14, TAR15-15, TAR15-16, TAR15-17, TAR15-18, TAR15-19, TAR15-20, TAR 15-22, TAR15-5, TAR15-6, TAR15-7, TAR15-8, TAR15-23, TAR15-24, TAR15-25, TAR15-26, TAR15-27, TAR15-29, and TAR15-30.

The invention further encompasses a composition comprising a single domain antibody polypeptide construct that antagonizes human VEGF binding to a receptor, wherein the single domain antibody polypeptide construct comprises an amino acid sequence selected from the group consisting of TAR15-1, TAR15-3, TAR15-4, TAR15-9, TAR15-10, TAR15-11, TAR15-12, TAR15-13, TAR15-14, TAR15-15, TAR15-16, TAR15-17, TAR15-18, TAR15-19, TAR15-20, TAR 15-22, TAR15-5, TAR15-6, TAR15-7, TAR15-8, TAR15-23, TAR15-24, TAR15-25, TAR15-26, TAR15-27, TAR15-29, and TAR15-30 or a sequence at least 85% identical thereto.

The invention further encompasses a composition comprising a single domain antibody polypeptide construct that antagonizes human VEGF binding to a receptor, wherein the single domain antibody polypeptide construct comprises an amino acid sequence selected from the group consisting of TAR15-1, TAR15-3, TAR15-4, TAR15-9, TAR15-10, TAR15-11, TAR15-12, TAR15-13, TAR15-14, TAR15-15, TAR15-16, TAR15-17, TAR15-18, TAR15-19, TAR15-20, TAR 15-22, TAR15-5, TAR15-6, TAR15-7, TAR15-8, TAR15-23, TAR15-24, TAR15-25, TAR15-26, TAR15-27, TAR15-29, and TAR15-30 or a sequence at least 90% identical thereto.

The invention further encompasses a composition comprising a single domain antibody polypeptide construct that antagonizes human VEGF binding to a receptor, wherein the single domain antibody polypeptide construct comprises an amino acid sequence selected from the group consisting of TAR15-1, TAR15-3, TAR15-4, TAR15-9, TAR15-10, TAR15-11, TAR15-12, TAR15-13, TAR15-14, TAR15-15, TAR15-16, TAR15-17, TAR15-18, TAR15-19, TAR15-20, TAR 15-22, TAR15-5, TAR15-6, TAR15-7, TAR15-8, TAR15-23, TAR15-24, TAR15-25, TAR15-26, TAR15-27, TAR15-29, and TAR15-30 or a sequence at least 92% identical thereto.

The invention further encompasses a composition comprising a single domain antibody polypeptide construct that antagonizes human VEGF binding to a receptor, wherein the single domain antibody polypeptide construct comprises an amino acid sequence selected from the group consisting of TAR15-1, TAR15-3, TAR15-4, TAR15-9, TAR15-10, TAR15-11, TAR15-12, TAR15-13, TAR15-14, TAR15-15, TAR15-16, TAR15-17, TAR15-18, TAR15-19, TAR15-20, TAR 15-22, TAR15-5, TAR15-6, TAR15-7, TAR15-8, TAR15-23, TAR15-24, TAR15-25, TAR15-26, TAR15-27, TAR15-29, and TAR15-30 or a sequence at least 94% identical thereto.

The invention further encompasses a composition comprising a single domain antibody polypeptide construct that antagonizes human VEGF binding to a receptor, wherein the single domain antibody polypeptide construct comprises an amino acid sequence selected from the group consisting of TAR15-1, TAR15-3, TAR15-4, TAR15-9, TAR15-10, TAR15-11, TAR15-12, TAR15-13, TAR15-14, TAR15-15, TAR15-16, TAR15-17, TAR15-18, TAR15-19, TAR15-20, TAR 15-22, TAR15-5, TAR15-6, TAR15-7, TAR15-8, TAR15-23, TAR15-24, TAR15-25, TAR15-26, TAR15-27, TAR15-29, and TAR15-30 or a sequence at least 96% identical thereto.

The invention further encompasses a composition comprising a single domain antibody polypeptide construct that antagonizes human VEGF binding to a receptor, wherein the single domain antibody polypeptide construct comprises an amino acid sequence selected from the group consisting of TAR15-1, TAR15-3, TAR15-4, TAR15-9, TAR15-10, TAR15-11, TAR15-12, TAR15-13, TAR15-14, TAR15-15, TAR15-16, TAR15-17, TAR15-18, TAR15-19, TAR15-20, TAR 15-22, TAR15-5, TAR15-6, TAR15-7, TAR15-8, TAR15-23, TAR15-24, TAR15-25, TAR15-26, TAR15-27, TAR15-29, and TAR15-30 or a sequence at least 98% identical thereto.

The invention further encompasses a composition comprising a single domain antibody polypeptide construct that antagonizes human VEGF binding to a receptor, wherein the single domain antibody polypeptide construct comprises an amino acid sequence selected from the group consisting of TAR15-1, TAR15-3, TAR15-4, TAR15-9, TAR15-10, TAR15-11, TAR15-12, TAR15-13, TAR15-14, TAR15-15, TAR15-16, TAR15-17, TAR15-18, TAR15-19, TAR15-20, TAR 15-22, TAR15-5, TAR15-6, TAR15-7, TAR15-8, TAR15-23, TAR15-24, TAR15-25, TAR15-26, TAR15-27, TAR15-29, and TAR15-30 or a sequence at least 99% identical thereto.

The invention further encompasses a method of treating rheumatoid arthritis, the method comprising administering to an individual in need thereof a therapeutically effective amount of a composition, wherein the composition comprises a single domain antibody polypeptide construct that antagonizes human TNFα's binding to a receptor and antagonizes human VEGF's binding to a receptor, whereby the rheumatoid arthritis is treated.

In one embodiment, the composition prevents an increase in arthritic score when administered to a mouse of the Tg197 transgenic mouse model of arthritis.

In another embodiment, the administration of the composition to a Tg197 transgenic mouse comprises the following steps: a) administer weekly intraperitoneal injections of the composition to a heterozygous Tg197 transgenic mouse, b) weigh the mouse of step a) weekly, and c) score the mouse weekly for macrophenotypic signs of arthritis according to the following system: 0=no arthritis (normal appearance and flexion), 1=mild arthritis (joint distortion), 2=moderate arthritis (swelling, joint deformation), 3=heavy arthritis (severely impaired movement).

In another embodiment, the composition has an efficacy in the Tg197 transgenic mouse arthritis assay that is greater than or equal, within the realm of statistical significance, to that of an agent selected from the group consisting of Etanercept, Infliximab and D2E7.

In another embodiment, the treating comprises inhibiting the progression of the rheumatoid arthritis.

In another embodiment, the treating comprises preventing or delaying the onset of rheumatoid arthritis.

In another embodiment, the administering results in a statistically significant change in one or more indicia of RA.

In another embodiment, the one or more indicia of RA comprise one or more of erythrocyte sedimentation rate (ESR), Ritchie articular index and duration of morning stiffness, joint mobility, joint swelling, x ray imaging of one or more joints, and histopathological analysis of fixed sections of one or more joints.

In another embodiment, the one or more indicia of RA comprises a decrease in the macrophenotypic signs of arthritis in a Tg197 transgenic mouse, wherein the composition is administered to a Tg197 transgenic mouse, wherein the Tg197 transgenic mouse is scored for the macrophenotypic signs of arthritis, and wherein the macrophenotypic signs of arthritis are scored according to the following system: 0=no arthritis (normal appearance and flexion), 1=mild arthritis (joint distortion), 2=moderate arthritis (swelling, joint deformation), 3=heavy arthritis (severely impaired movement).

In another embodiment, the one or more indicia of RA comprises a decrease in the histopathological signs of arthritis in a Tg197 transgenic mouse, wherein the composition is administered to a Tg197 transgenic mouse, wherein the Tg197 transgenic mouse is scored for the histopathological signs of arthritis, and wherein the histopathological signs of arthritis are performed on a joint and scored using the following system: 0=no detectable pathology, 1=hyperplasia of the synovial membrane and presence of polymorphonuclear infiltrates, 2=pannus and fibrous tissue formation and focal subchondral bone erosion, 3=articular cartilage destruction and bone erosion, 4=extensive articular cartilage destruction and bone erosion.

In another embodiment, the single domain antibody polypeptide construct comprises a human single domain antibody polypeptide.

In another embodiment, the human single domain antibody polypeptide binds TNFα and VEGF.

In another embodiment, the single domain antibody polypeptide construct neutralizes human TNFα as measured in a standard L929 cell assay.

In another embodiment, the single domain antibody polypeptide construct is linked to a PEG molecule.

In another embodiment, the PEG-linked single domain antibody polypeptide construct has a hydrodynamic size of at least 24 kDa, and wherein the total PEG size is from 20 to 60 kDa.

In another embodiment, the PEG-linked single domain antibody polypeptide construct has a hydrodynamic size of at least 200 kDa and a total PEG size of from 20 to 60 kDa.

In another embodiment, the antibody polypeptide construct is linked, on average, to 1, 2, 3, 4, 5, 6, 7, 8, 9, 10, 12, 15, 17, 20, or more polyethylene glycol molecules.

In another embodiment, the antibody construct comprises two or more single immunoglobulin variable domain polypeptides that bind human TNFα and/or two or more single immunoglobulin variable domain polypeptides that bind human VEGF.

In another embodiment, the antibody construct comprises a homodimer of a single immunoglobulin variable domain polypeptide that binds human TNFα and/or a homodimer of a single immunoglobulin variable domain polypeptide that binds human VEGF.

In another embodiment, the antibody construct comprises a homotrimer of a single immunoglobulin variable domain polypeptide that binds human TNFα and/or a homotrimer of a single immunoglobulin variable domain polypeptide that binds human VEGF.

In another embodiment, the antibody construct comprises a homotetramer of a single immunoglobulin variable domain polypeptide that binds human TNFα and/or a homotetramer of a single immunoglobulin variable domain polypeptide that binds human VEGF.

In another embodiment, the construct further comprises an antibody polypeptide specific for an antigen other than TNFα or VEGF.

In another embodiment, the antibody polypeptide specific for an antigen other than TNFα or VEGF comprises a single domain antibody polypeptide.

In another embodiment, the binding of the antigen other than TNFα or VEGF by the antibody polypeptide specific for an antigen other than TNFα or VEGF increases the in vivo half-life of the antibody polypeptide construct.

In another embodiment, the antigen other than TNFα or VEGF comprises a serum protein.

In another embodiment, the serum protein is selected from the group consisting of fibrin, α-2 macroglobulin, serum albumin, fibrinogen A, fibrinogen, serum amyloid protein A, heptaglobin, protein, ubiquitin, uteroglobulin and β-2-microglobulin.

In another embodiment, the antigen other than TNFα comprises HSA.

In another embodiment, the treating further comprises administration of at least one additional therapeutic agent.

The invention further encompasses a composition comprising a single domain antibody polypeptide construct that antagonizes human TNFα's binding to a receptor and that antagonizes human's VEGF's binding to a receptor, that prevents an increase in arthritic score when administered to a mouse of the Tg197 transgenic mouse model of arthritis, wherein the single domain antibody polypeptide construct inhibits the progression of the rheumatoid arthritis.

The invention further encompasses a composition comprising a single domain antibody polypeptide construct that antagonizes human TNFα's binding to a receptor and that antagonizes human's VEGF's binding to a receptor, that prevents an increase in arthritic score when administered to a mouse of the Tg197 transgenic mouse model of arthritis, wherein the single domain antibody polypeptide construct binds human TNFα with a Kd of <100 nM.

The invention further encompasses a composition comprising a single domain antibody polypeptide construct that antagonizes human TNFα's binding to a receptor and that antagonizes human's VEGF's binding to a receptor, that prevents an increase in arthritic score when administered to a mouse of the Tg197 transgenic mouse model of arthritis, wherein the single domain antibody polypeptide construct neutralizes human TNFα as measured in a standard L929 cell assay, wherein the single domain antibody polypeptide construct inhibits the progression of the rheumatoid arthritis, wherein the single domain antibody polypeptide construct binds human TNFα with a Kd of <100 nM.

Another aspect is a method for selecting a single domain antibody polypeptide construct that antagonizes human TNFα's binding to a receptor, that prevents an increase in arthritic score when administered to a mouse of the Tg197 transgenic mouse model of arthritis, wherein said single domain antibody polypeptide construct neutralizes human TNFα as measured in a standard L929 cell assay, wherein said single domain antibody polypeptide construct inhibits the progression of said rheumatoid arthritis, and wherein said single domain antibody polypeptide construct binds human TNFα with a Kd of <100 nM, comprising the following steps: (1) mutating nucleic acid encoding several hypervariable region sites of said single domain antibody polypeptide construct, so that all possible amino substitutions are generated at each site, (2) introducing nucleic acid encoding the mutated hypervariable region sites generated in step (1) into a phagemid display vector, to form a large population of display vectors each capable of expressing one of said mutated hypervariable region sites displayed on a phagemid surface display protein; (3) expressing the mutated hypervariable region sites on the surface of a filamentous phage particle so that the mutated hypervariable region sites thus generated are displayed in a monovalent fashion from filamentous phage particles as fusions to the gene III product of M13 packaged within each particle, (4) screening the surface-expressed phage particle for the ability to bind TNFa, (5) isolating those surface-expressed phage particle able to bind TNFa, (6) selecting a surface-expressed phage particle from step (5) that is able to bind TNFa, that also prevents an increase in arthritic score when administered to a mouse of the Tg197 transgenic mouse model of arthritis, and neutralizes human TNFα as measured in a standard L929 cell assay, and inhibits the progression of said rheumatoid arthritis, and binds human TNFα with a Kd of <100 nM, thereby selecting one or more species of phagemid containing a display protein containing a single domain antibody polypeptide construct that antagonizes human TNFα's binding to a receptor.

Another aspect is a method of treating rheumatoid arthritis, the method comprising administering to an individual in need thereof a therapeutically effective amount of a composition comprising a single domain antibody polypeptide construct that antagonizes human VEGF's binding to a receptor, whereby said rheumatoid arthritis is treated, wherein said single domain antibody polypeptide construct has an in vivo to-half life in the range of 15 minutes to 12 hours, 1 to 6 hours, 2 to 5 hours, or 3 to 4 hours.

Another embodiment is a method of treating rheumatoid arthritis, the method comprising administering to an individual in need thereof a therapeutically effective amount of a composition comprising a single domain antibody polypeptide construct that antagonizes human VEGF's binding to a receptor, whereby said single domain antibody polypeptide construct has an in vivo tβ-half life in the range of 12 to 60 hours, 12 to 48 hours, or 12 to 26 hours.

Another embodiment is method of treating rheumatoid arthritis, the method comprising administering to an individual in need thereof a therapeutically effective amount of a composition comprising a single domain antibody polypeptide construct that antagonizes human VEGF's binding to a receptor, whereby said single domain antibody polypeptide construct has an in vivo AUC half-life value of 15 mg.min/ml to 150 mg.min/ml, 15 mg.min/ml to 100 mg.min/ml, 15 mg.min/m1 to 75 mg.min/ml, or 15 mg.min/ml to 50 mg.min/ml.

Another embodiment is a method of treating rheumatoid arthritis, the method comprising administering to an individual in need thereof a therapeutically effective amount of a composition, wherein said composition comprises a single domain antibody polypeptide construct that antagonizes human TNFα's binding to a receptor and antagonizes human VEGF's binding to a receptor, whereby said rheumatoid arthritis is treated, and wherein said composition prevents an increase in arthritic score when administered to a mouse of the Tg197 transgenic mouse model of arthritis, and wherein said single domain antibody polypeptide construct binds human TNFα and VEGF each with a Kd of <100 nM, wherein said single domain antibody polypeptide construct binds human TNFα and VEGF each with a Kd in the range of 100 nM to 50 pM, wherein said single domain antibody polypeptide construct binds human TNFα and VEGF each with a K_{d} of 30 nM to 50 pM, wherein said single domain antibody polypeptide construct binds human TNFα and VEGF each with a Kd of 10 nM to 50 pM, or wherein said single domain antibody polypeptide construct binds human TNFα and VEGF each with a K_{d} in the range of 1 nm to 50 pM.

Another embodiment is a method of treating rheumatoid arthritis, the method comprising administering to an individual in need thereof a therapeutically effective amount of a composition comprising a single domain antibody polypeptide construct that antagonizes human TNFα's binding to a receptor and antagonizes VEGF's binding to a receptor, wherein said single domain antibody polypeptide construct inhibits the binding of human TNFα to a TNFα receptor and of human VEGF to a VEGF receptor, and whereby said rheumatoid arthritis is treated.

Another embodiment is a method of treating rheumatoid arthritis, the method comprising administering to an individual in need thereof a therapeutically effective amount of a composition comprising a single domain antibody polypeptide construct that antagonizes human TNFα's binding to a receptor and antagonizes VEGF's binding to a receptor, wherein said single domain antibody polypeptide construct inhibits the binding of human TNFα to a TNFα receptor and of human VEGF to a VEGF receptor, and whereby said rheumatoid arthritis is treated, wherein said single domain antibody polypeptide construct specifically binds to human TNFα which is bound to a cell surface receptor.

Another embodiment is a method of treating rheumatoid arthritis, the method comprising administering to an individual in need thereof a therapeutically effective amount of a composition, wherein said composition comprises a single domain antibody polypeptide construct that antagonizes human TNFα's binding to a receptor and antagonizes human VEGF's binding to a receptor, whereby said rheumatoid arthritis is treated, and wherein said single domain antibody polypeptide construct specifically binds to human TNFα which is bound to a cell surface receptor.

Another embodiment of the invention is a method of treating rheumatoid arthritis comprising the administration of an antibody construct specific for TNFα, wherein the sequence of the antibody construct comprises, or consists of, a sequence with a percentage identity which is greater than or equal to 85, 90, 95, 96, 97, 98, 99 or 100% to the sequence of any one of the anti-TNF-α clones recited herein. Another embodiment of the invention is a composition comprising an antibody construct specific for TNFα, wherein the sequence of the antibody construct comprises, or consists of, a sequence with a percentage identity which is greater than or equal to 85, 90, 95, 96, 97, 98, 99 or 100% to the sequence of any one of the anti-TNF-α clones recited herein. Another embodiment of the invention is a method of treating rheumatoid arthritis comprising the administration of an antibody construct specific for VEGF, wherein the sequence of the antibody construct comprises, or consists of a sequence with a percentage identity which is greater than or equal to 85, 90, 95, 96, 97, 98, 99 or 100% to the sequence of any one of the anti-VEGF clones recited herein. Another embodiment of the invention is a composition comprising an antibody construct specific for VEGF, wherein the sequence of the antibody construct comprises, or consists of, a sequence with a percentage identity which is greater than or equal to 85, 90, 95, 96, 97, 98, 99 or 100% to the sequence of any one of the anti-VEGF clones recited herein.

In another embodiment, there are provided tetravalent, dual-specific antigen-binding polypeptide constructs comprising two copies of a V_{H} or V_{L} single domain antibody that binds a first antigen or epitope; and two copies of a V_{H} or V_{L} single domain antibody that binds a second antigen or epitope. The first and second epitopes can be present on the same antigen or, alternatively, on different antigens. Each of the two copies of the single domain antibody that binds the first antigen or epitope is fused to a respective IgG heavy chain constant domain, and each of the two copies of the single domain antibody that binds the second antigen or epitope is fused to a respective light chain constant domain. These tetravalent, dual-specific polypeptide constructs are IgG-like in that they have two antigen-binding arms joined by heavy and light chain constant domains. They are different from naturally-occurring IgG in that, by virtue of the presence of two different antigen-specific single domain antibody polypeptides on each arm, each arm can bind two different antigens or epitopes, making the construct tetravalent and dual-specific. In one embodiment, the first and second epitopes are the same, such that there are four specific binding sites for that epitope present on the polypeptide construct. In another embodiment, the first and second epitopes are different, being present on the same or different antigens.

Dual-specific, tetravalent polypeptide constructs as described herein can include single domain antibody sequences specific for any two antigens or epitopes, but particularly those specific for human TNF-α and VEGF, and more particularly, any of those single domain antibody sequences described herein. In other embodiments, C_{κ} or C_{λ} light chain constant domains can be used, and IgG heavy chain constant domains other than IgG1 can also be used.

Also encompassed are constructs of this sort comprising single domain anti-TNF-α antibody clones that prevent an increase in arthritic score when administered as a monomer to a mouse of the Tg197 transgenic mouse model of arthritis, and single domain anti-VEGF antibody clones that prevent an increase in arthritic score when administered as a monomer to a mouse of a collagen-induced arthritis mouse model. In a further embodiment, the single domain anti-TNF-α antibody clone used neutralizes human TNF-α n the L929 cell cytotoxicity assay described herein when used as a monomer, and the single domain anti-VEGF antibody clone used antagonizes VEGF receptor binding in an assay of VEGF Receptor 2 binding as described herein when used a monomer. In a further embodiment, the single domain antibody clones used bind their respective antigens or epitopes with a K_{d} of <100 nM. In a further embodiment, the dual-specific, bi-valent constructs bind the respective antigens or epitopes with a K_{d} of <100 nM and prevent an increase in arthritic score in either or both of the Tg197 and CIA models of arthritis described herein.

Such tetravalent, dual specific constructs can be used for the treatment of rheumatoid arthritis in a manner similar to the other constructs described herein, in terms of administration, dosage and monitoring of efficacy. The half-life of the construct can be modified as described herein above, e.g., by addition of a PEG moiety, or by further fusion of a binding moiety (e.g., a further single domain antibody) specific for a protein that increases circulating half-life, e.g., a serum protein such as HSA.

### BRIEF DESCRIPTION OF THE FIGURES

FIG. 1 shows the diversification of V_{H}/HSA at positions HSO, H52, H52a, H53, H55, H56, H58, H95, H96, H97, H98 (DVT or NNK encoded respectively) which are in the antigen binding site of V_{H} HSA. The sequence of V_{K} is diversified at positions L50, L53.

FIG. 2 shows Library 1: Germline V_{K}/DVT V_{H},
Library 2: Germline V_{K} /NNK V_{H},
Library 3: Germline V_{H}/DVT V_{K}
Library 4: Germline V_{H}/NNK V_{K}

In phage display/ScFv format. These libraries were pre-selected for binding to generic ligands protein A and protein L so that the majority of the clones and selected libraries are functional. Libraries were selected on HSA (first round) and β-gal (second round) or HSA β-gal selection or on β-gal (first round) and HSA (second round) β-gal HSA selection. Soluble scFv from these clones of PCR are amplified in the sequence. One clone encoding a dual specific antibody K8 was chosen for further work.

FIG. 3 shows an alignment of V_{H} chains and V_{κ} chains.

FIG. 4 shows the characterisation of the binding properties of the K8 antibody, the binding properties of the K8 antibody characterised by monoclonal phage ELISA, the dual specific K8 antibody was found to bind HSA and β-gal and displayed on the surface of the phage with absorbant signals greater than 1.0. No cross reactivity with other proteins was detected.

FIG. 5 shows soluble scFv ELISA performed using known concentrations of the K8 antibody fragment. A 96-well plate was coated with 100 μg of HSA, BSA and β-gal at 10 μg/ml and 100 μg/ml of Protein A at 1 μg/ml concentration. 50 μg of the serial dilutions of the K8 scFv was applied and the bound antibody fragments were detected with Protein L-HRP. ELISA results confirm the dual specific nature of the K8 antibody.

FIG. 6 shows the binding characteristics of the clone K8V_{K}/dummy V_{H} analysed using soluble scFv ELISA. Production of the soluble scFv fragments was induced by IPTG as described by Harrison et al, Methods Enzymol. 1996;267:83-109 and the supernatant containing scFv assayed directly. Soluble scFv ELISA is performed as described in example 1 and the bound scFvs were detected with Protein L-HRP. The ELISA results revealed that this clone was still able to bind β-gal, whereas binding BSA was abolished.

FIG. 7 shows the sequence of variable domain vectors 1 and 2.

FIG. 8 is a map of the C_{H} vector used to construct a V_{H}1/V_{H}2 multipsecific ligand.

FIG. 9 is a map of the V_{κ} vector used to construct a V_{κ}1/V_{κ}2 multispecific ligand.

FIG. 10 TNF receptor assay comparing TAR1-5 dimer 4, TAR1-5-19 dimer 4 and TAR1-5-19 monomer.

FIG. 11 TNF receptor assay comparing TAR1-5 dimers 1-6.All dimers have been FPLC purified and the results for the optimal dimeric species are shown.

FIG. 12 TNF receptor assay of TAR1-5 19 homodimers in different formats: dAb-linker-dAb format with 3U, 5U or 7U linker, Fab format and cysteine hinge linker format.

FIG. 13 Dummy VH sequence for library 1. The sequence of the VH framework based on germline sequence DP47-JH4b. Positions where NNK randomisation (N=A or T or C or G nucleotides; K=G or T nucleotides) has been incorporated into library I are indicated in bold underlined text.

FIG. 14 Dummy VH sequence for library 2. The sequence of the VH framework based on germline sequence DP47-JH4b. Positions where NNK randomisation (N=A or T or C or G nucleotides; K=G or T nucleotides) has been incorporated into library 2 are indicated in bold underlined text.

FIG. 15 Dummy Vκ sequence for library 3. The sequence of the Vκ framework based on germline sequence DP_{K}9-J _{K}1. Positions where NNK randomisation (N=A or T or C or G nucleotides; K=G or T nucleotides) has been incorporated into library 3 are indicated in bold underlined text.

FIG. 16 Nucleotide and amino acid sequence of anti MSA dAbs MSA 16 and MSA 26.

FIG. 17 Inhibition biacore of MSA 16 and 26. Purified dAbs MSA16 and MSA26 were analysed by inhibition biacore to determine K_{d}. Briefly, the dAbs were tested to determine the concentration of dAb required to achieve 200RUs of response on a biacore CM5 chip coated with a high density of MSA. Once the required concentrations of dAb had been determined, MSA antigen at a range of concentrations around the expected K_{d} was premixed with the dAb and incubated overnight. Binding to the MSA coated biacore chip of dAb in each of the premixes was then measured at a high flow-rate of 30 μl/minute.

FIG. 18 Serum levels of MSA16 following injection. Serum half life of the dAb MSA16 was determined in mouse. MSA16 was dosed as single i.v. injections at approx 1.5 mg/kg into CD1 mice. Modelling with a 2 compartment model showed MSA16 had a t½α of 0.98 hr, a t½β of 36.5 hr and an AUC of 913 hr.mg/ml. MSA16 had a considerably lengthened half life compared with HEL4 (an anti-hen egg white lysozyme dAb) which had a t½α of 0.06 hr and a t½β of 0.34 hr.

FIG. 19 ELISA (a) and TNF receptor assay (c) showing inhibition of TNF binding with a Fab-like fragment comprising MSA26Ck and TAR1-5-19CH. Addition of MSA with the Fab-like fragment reduces the level of inhibition. An ELISA plate coated with 1 μg/ml TNFα was probed with dual specific VκC_{H} and VκCκ Fab like fragment and also with a control TNFα binding dAb at a concentration calculated to give a similar signal on the ELISA. Both the dual specific and control dAb were used to probe the ELISA plate in the presence and in the absence of 2 mg/ml MSA. The signal in the dual specific well was reduced by more than 50% but the signal in the dAb well was not reduced at all (see FIG. 19a). The same dual specific protein was also put into the receptor assay with and without MSA and competition by MSA was also shown (see FIG. 19c). This demonstrates that binding of MSA to the dual specific is competitive with binding to TNFα.

FIG. 20 TNF receptor assay showing inhibition of TNF binding with a disulphide bonded heterodimer of TAR1-5-19 dAb and MSA16 dAb. Addition of MSA with the dimer reduces the level of inhibiton in a dose dependant manner. The TNF receptor assay (FIG. 19(b)) was conducted in the presence of a constant concentration of heterodimer (18 nM) and a dilution series of MSA and HSA. The presence of HSA at a range of concentrations (up to 2 mg/ml) did not cause a reduction in the ability of the dimer to inhibit TNFα. However, the addition of MSA caused a dose dependant reduction in the ability of the dimer to inhibit TNFα (FIG. 19a).This demonstrates that MSA and TNFα compete for binding to the cys bonded TAR1-5-19, MSA16 dimer. MSA and HSA alone did not have an effect on the TNF binding level in the assay.

FIG. 21: Shows the vectors used for Fab construction according to the invention.

FIG. 22: Shows the binding of Fab comprising TAR1/TAR2 Dabs to TNF and TNFR1 via an ELISA assay.

FIG. 23: Shows the results of sandwich ELISA to test the ability of TAR1/TAR2 Fab to bind to both TNF and TNFR antigens simultaneously, that is to test whether the Fab is of open or closed conformation. FIG. 24: Shows the results of competition ELISA to test the ability of TAR1/TAR2 Fab to bind to both antigens simultaneously, that is to test whether the Fab is of open or closed conformation.

FIG. 25: Shows the results of cell based assays using Fab dual specific ligands according to the invention:

(a) to test human TNF cytotoxicity on murine cells

(b) shows a murine TNF cytotoxicity assay on murine cells with human soluble TAR2.

(c) Shows murine TNF induced IL-8 secretion on human cells.

(d) Shows human TNF induced IL-8 secretion on human cells.

FIG. 26: Shows murine TNF cytoxicity on murine cells with soluble human TNFR1 and increasing concentrations of mutant TNF (competition on cells).

FIG. 27: shows the construction of IgG vectors which express IgG1 heavy chain constant region and light chain kappa constant region respectively.

FIG. 28 shows the binding of TAR1/TAR2 IgG to TNF and TNFR1 in ELISA assay.

FIG. 29: Shows the analysis of TAR1/TAR2 IgG properties in cell assays.

(a) Human TNF cytotoxicity on murine cells.

(b) Murine TNF cytotoxicity assay on murine cells with human soluble TNF receptor.

(c) Human TNF induced IL-8 release from human cells.

(d) Murine TNF induced IL-8 secretion from human cells.

FIG. 30: Shows Human TNF induced IL-8 secretion on human cells

FIG. 31: Shows the amino acid sequence of the Dab designated TAR2 which binds to human TNFR1 (p55 receptor).

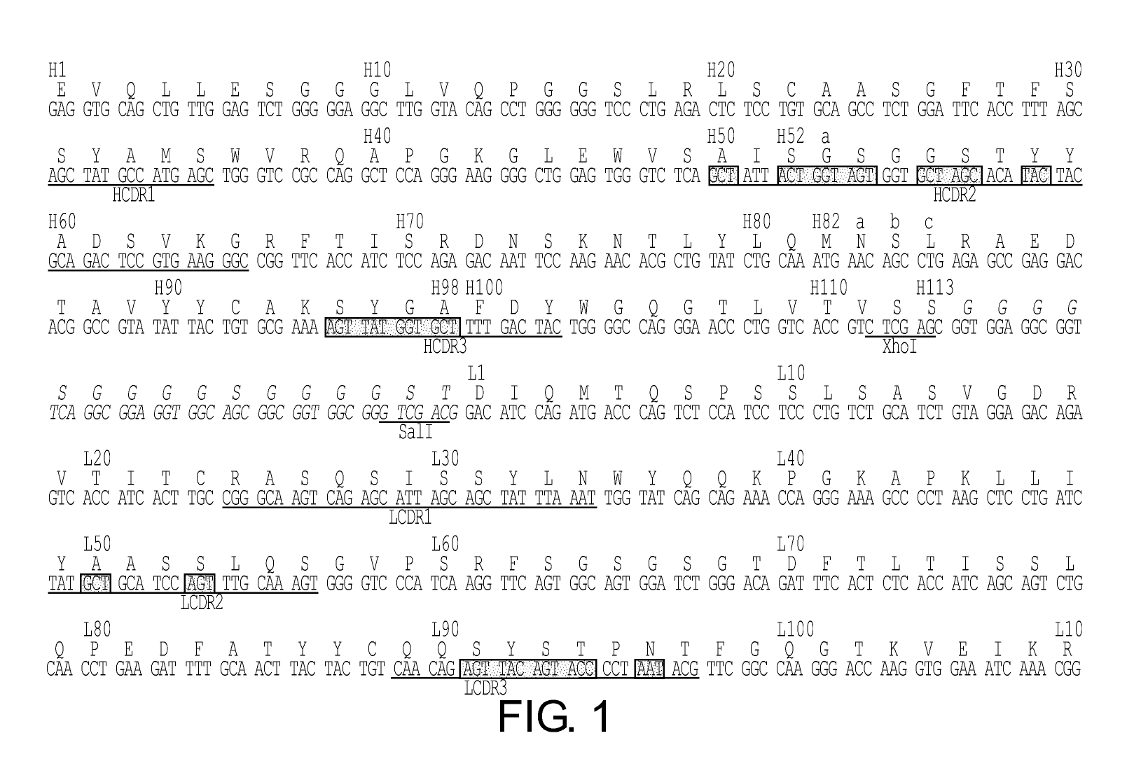

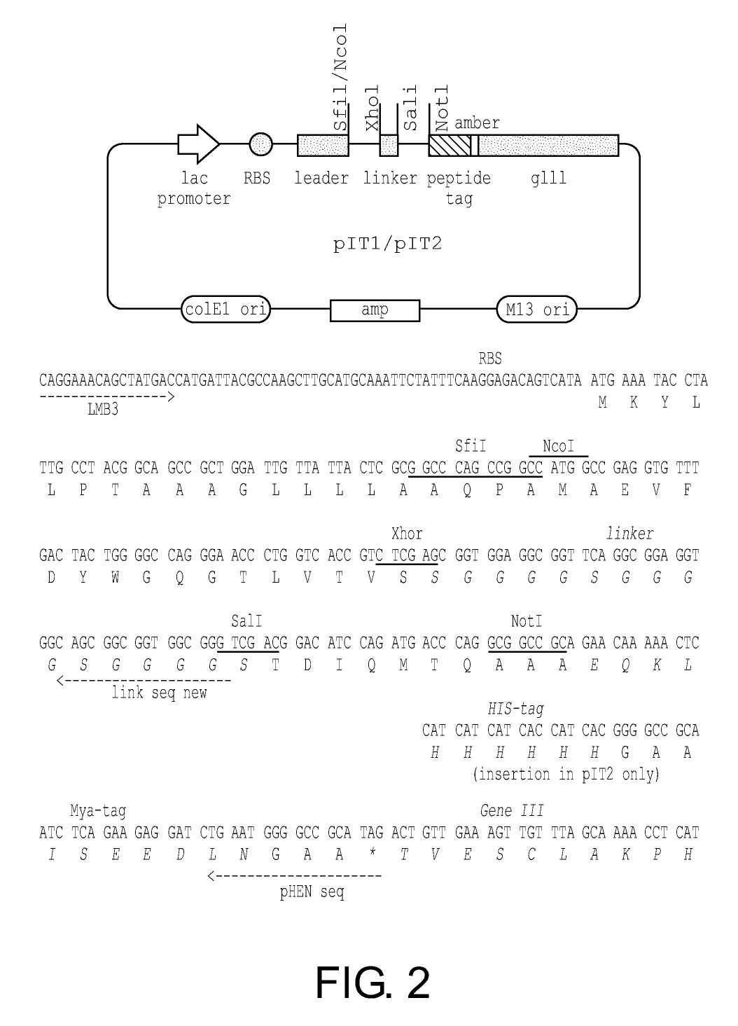

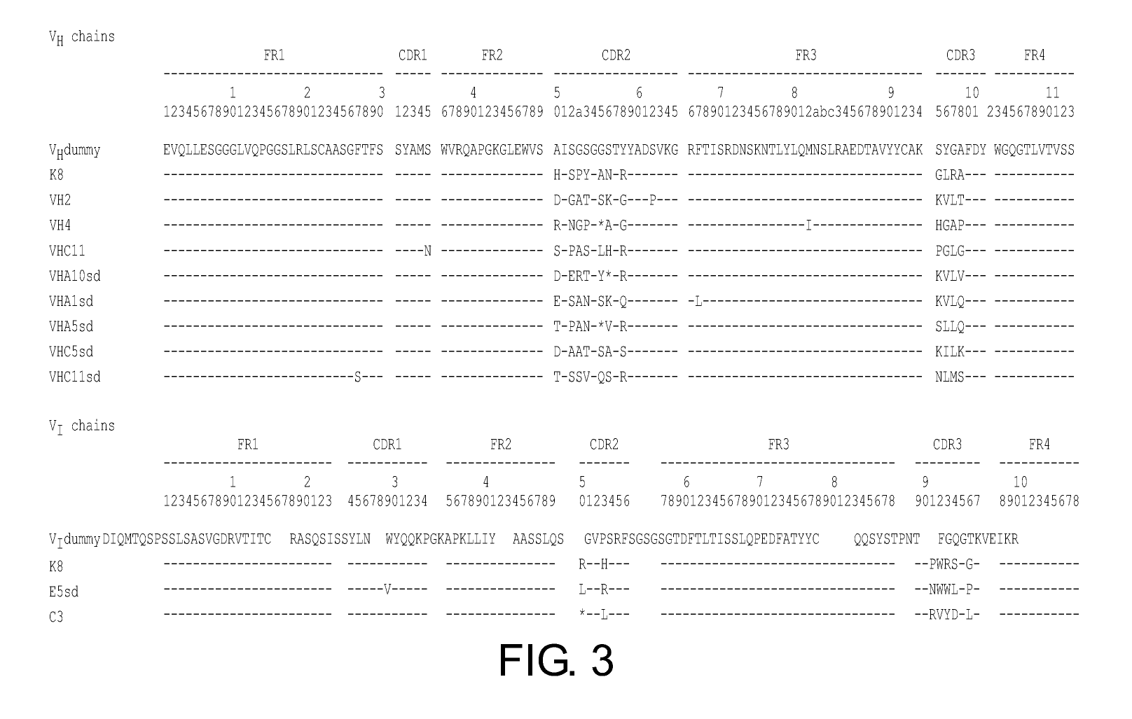

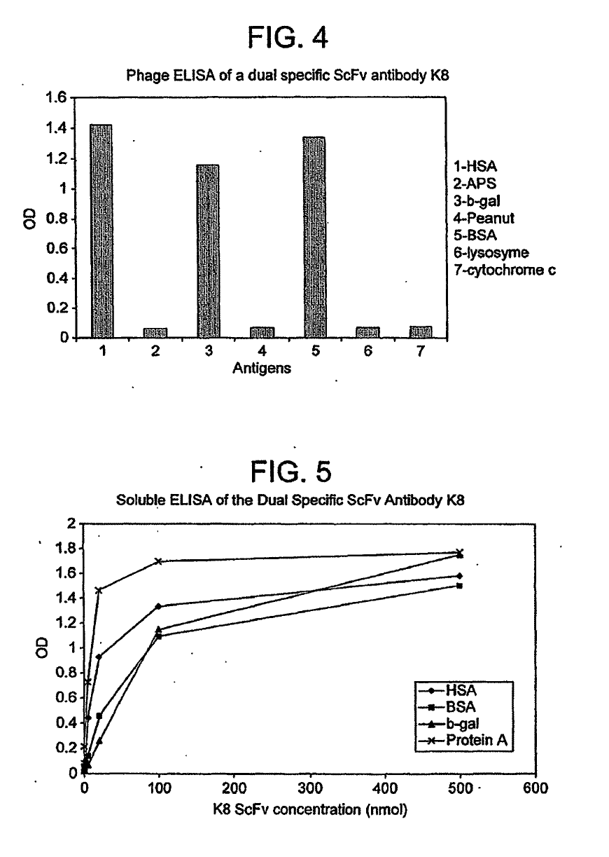

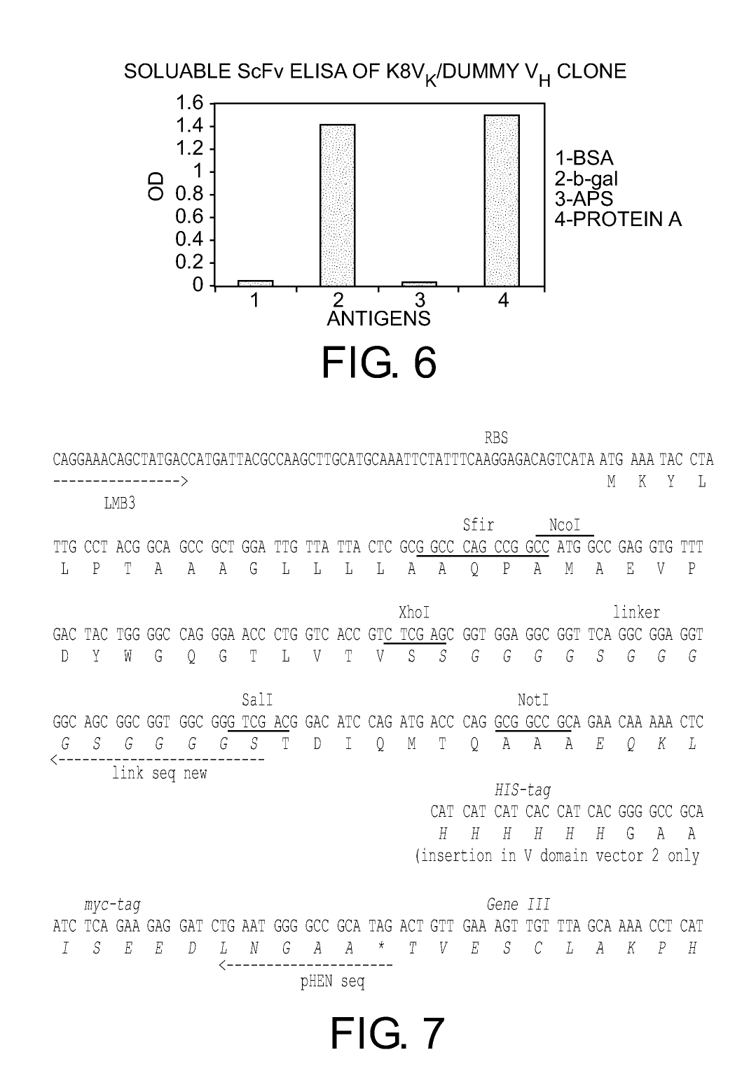

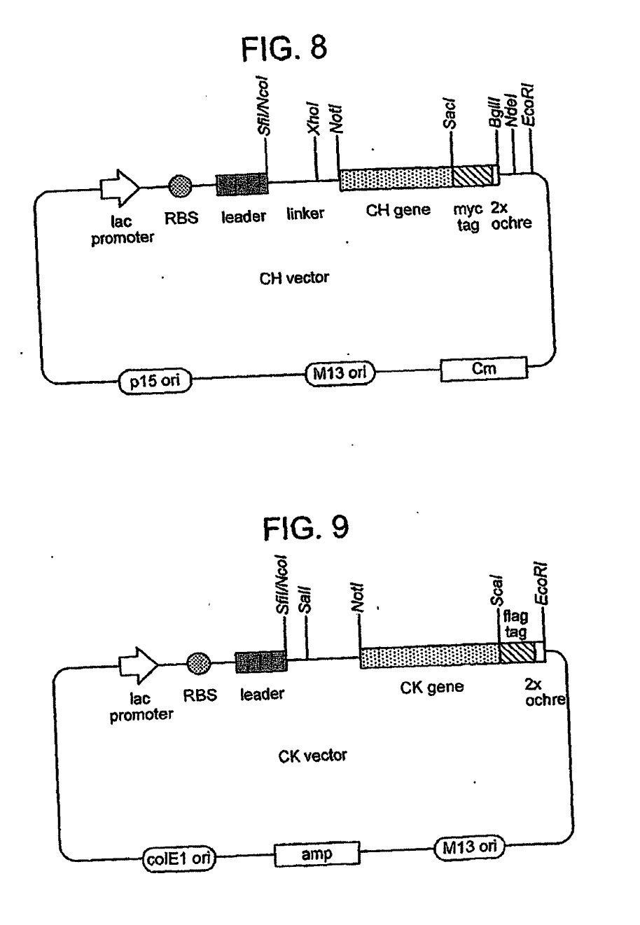

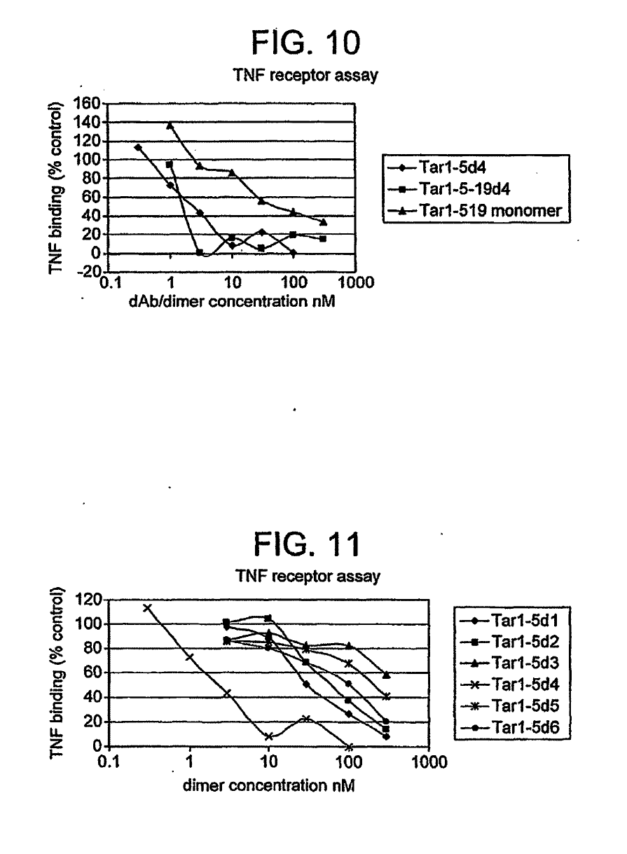

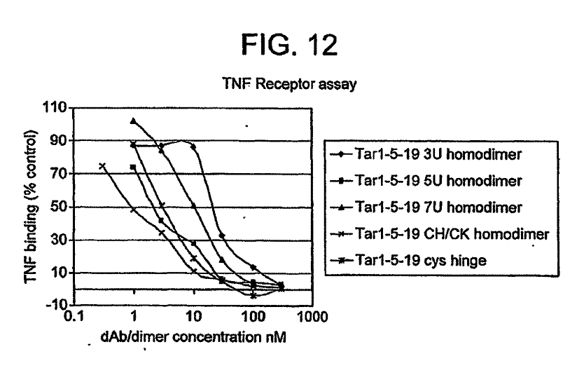

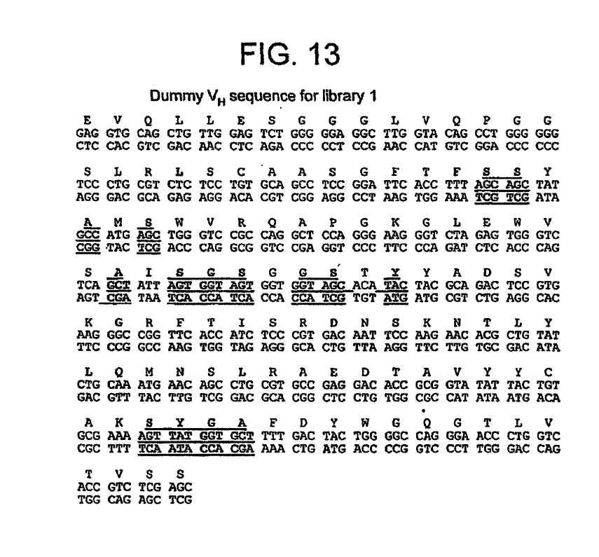

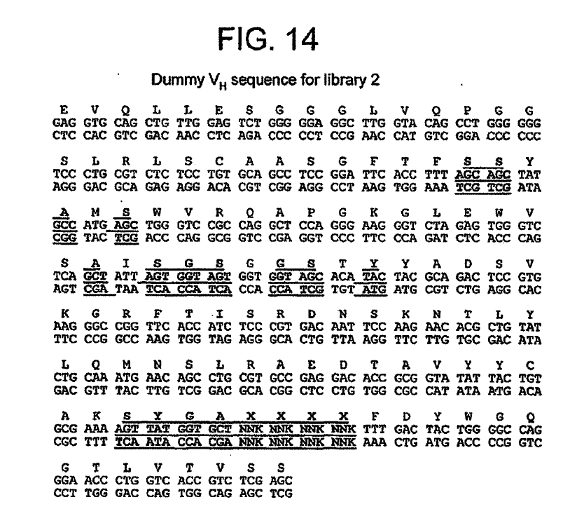

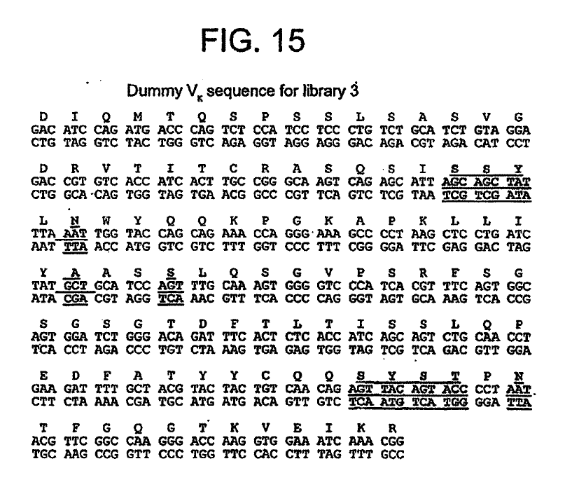

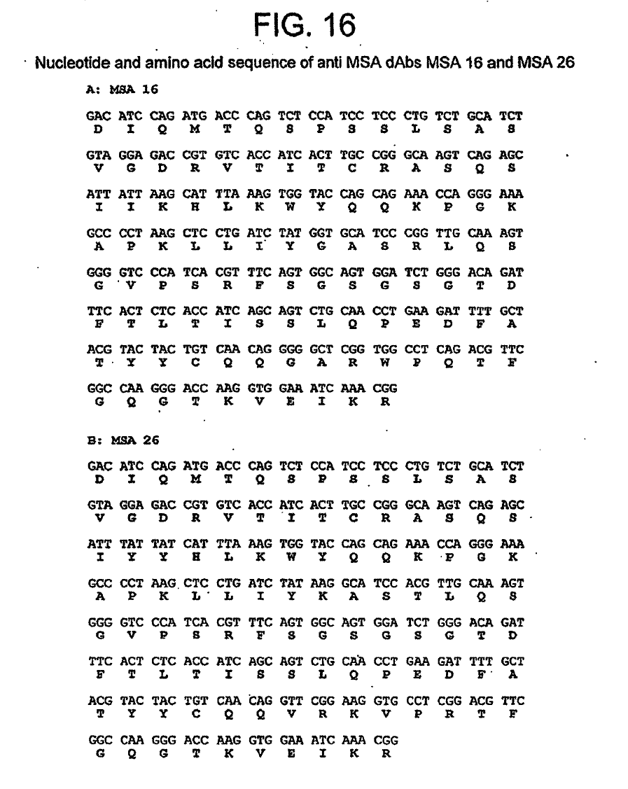

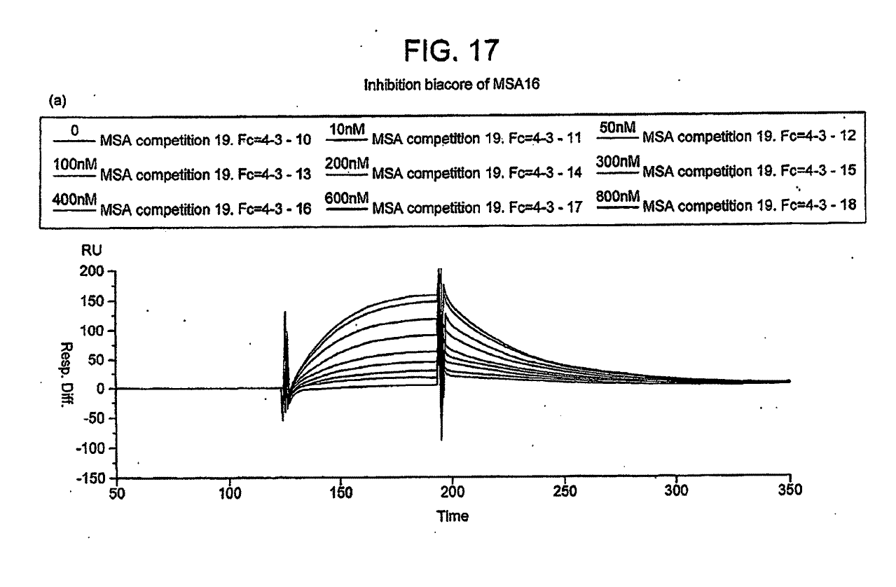

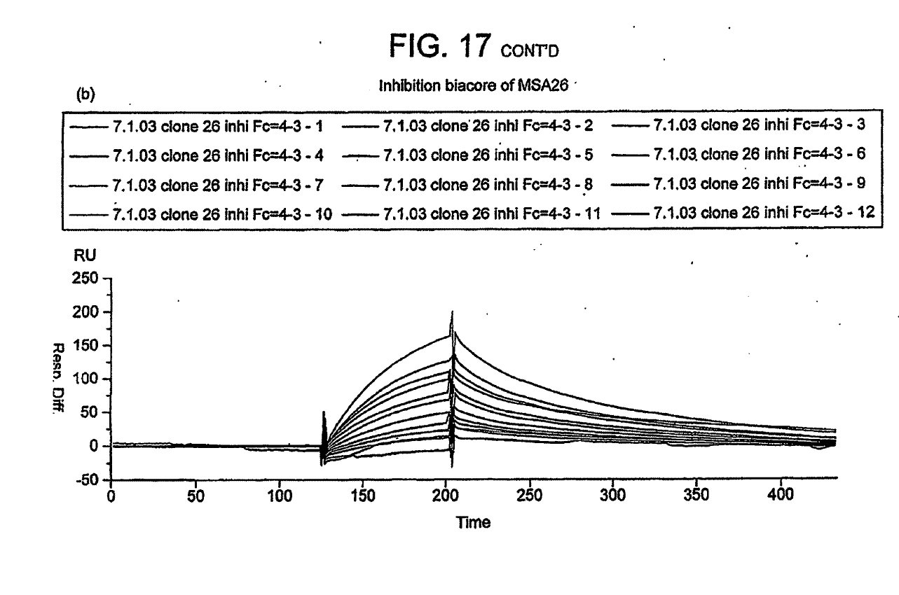

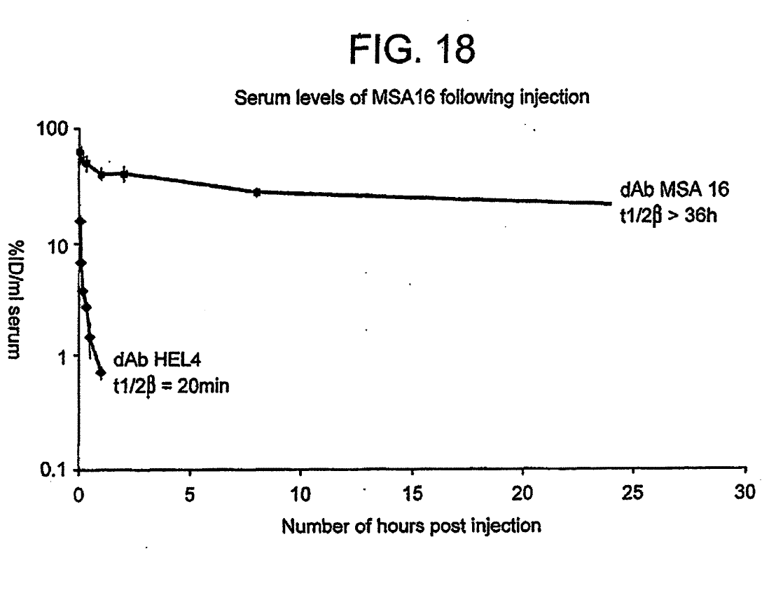

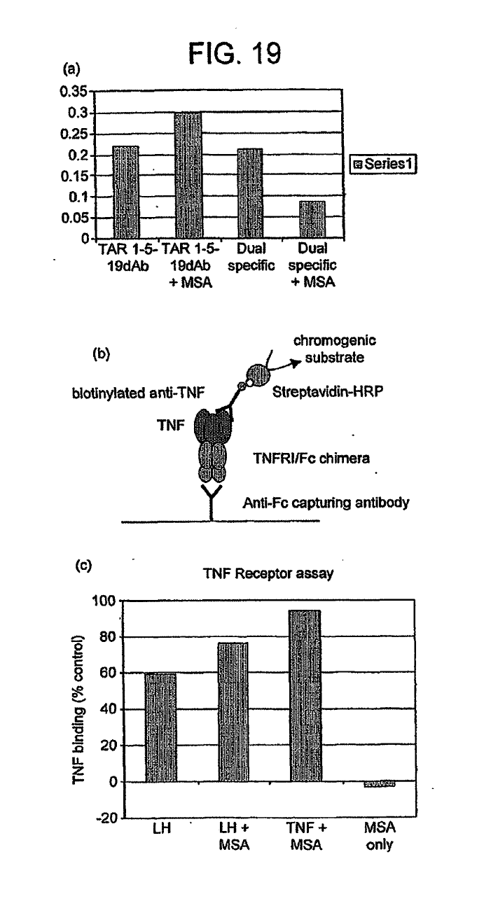

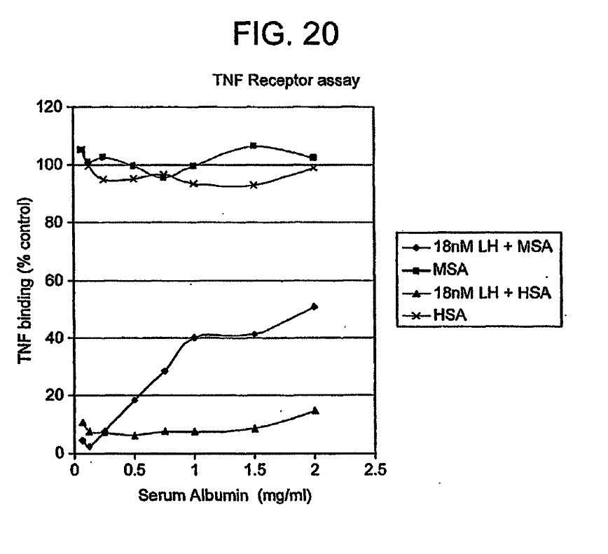

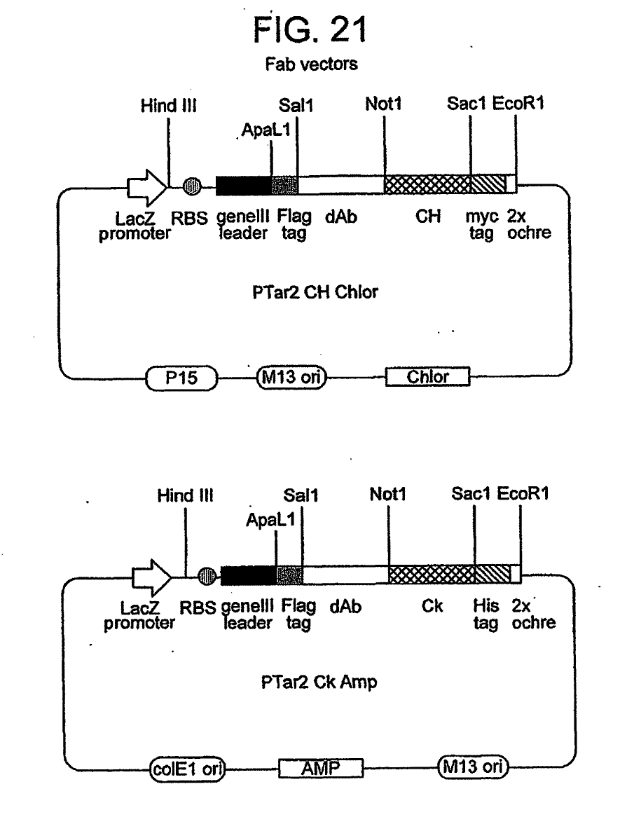

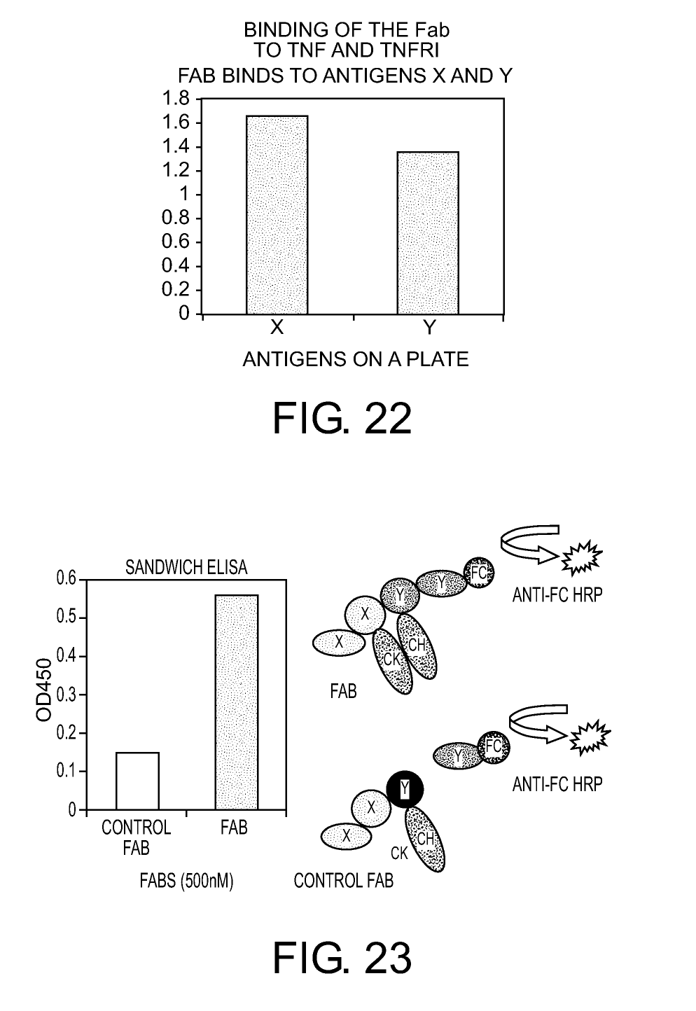

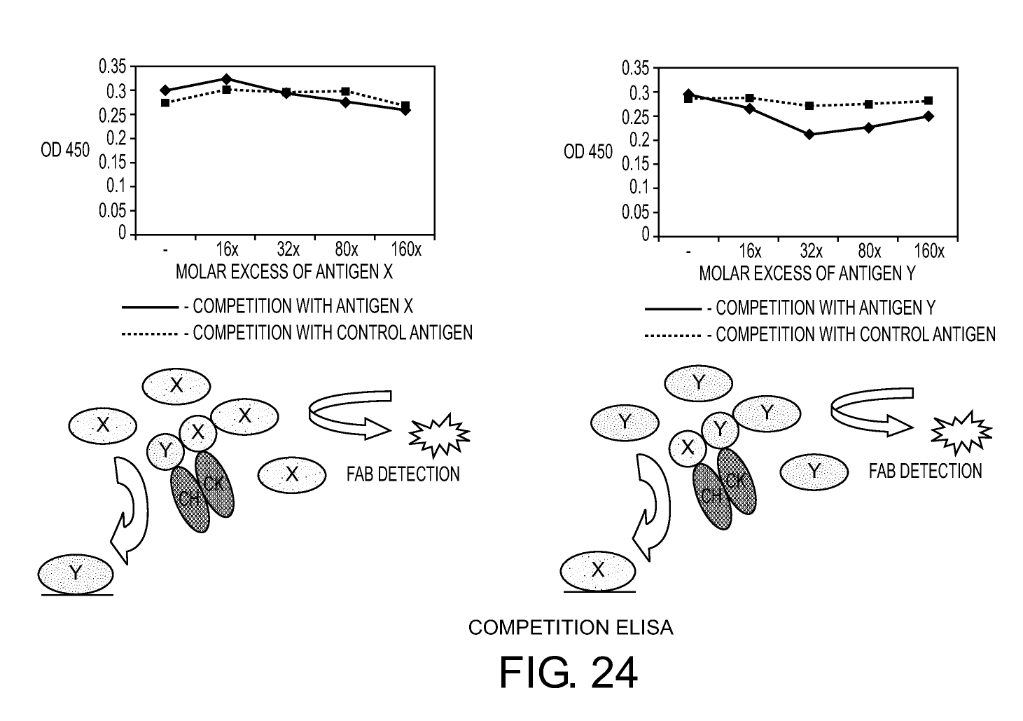

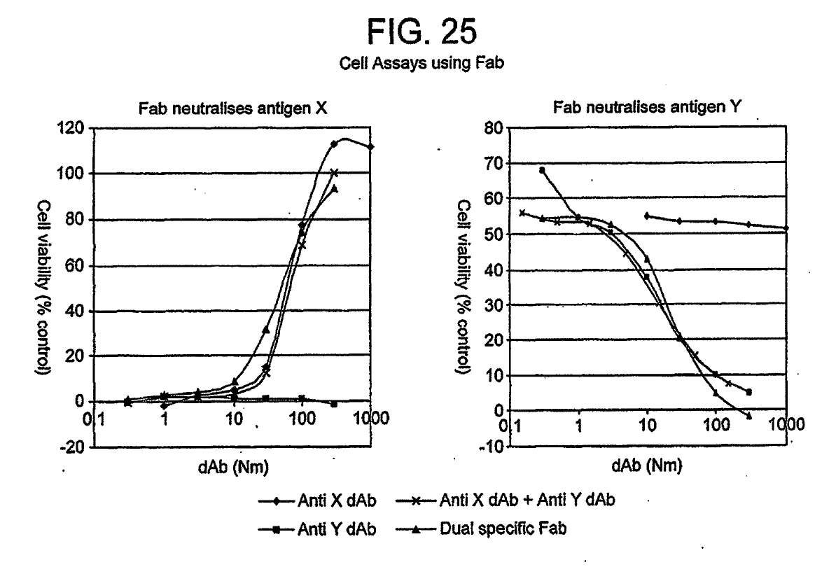

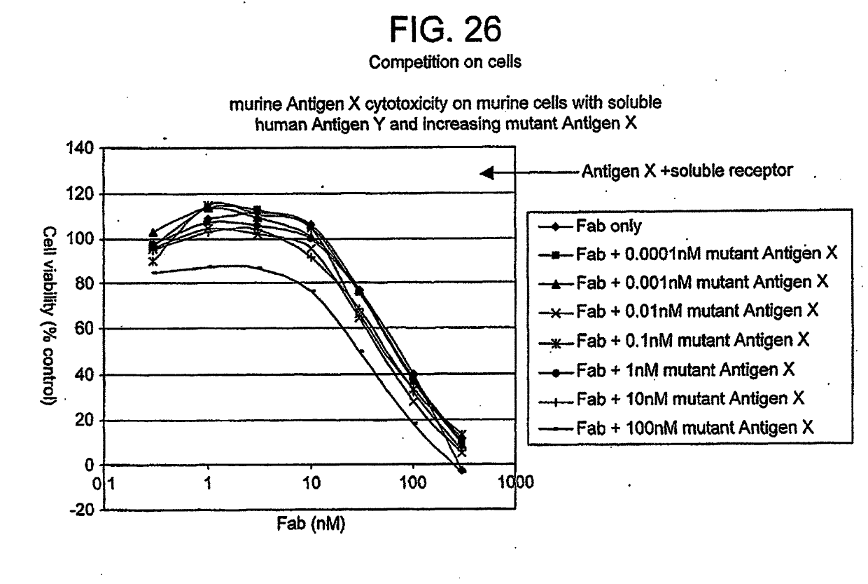

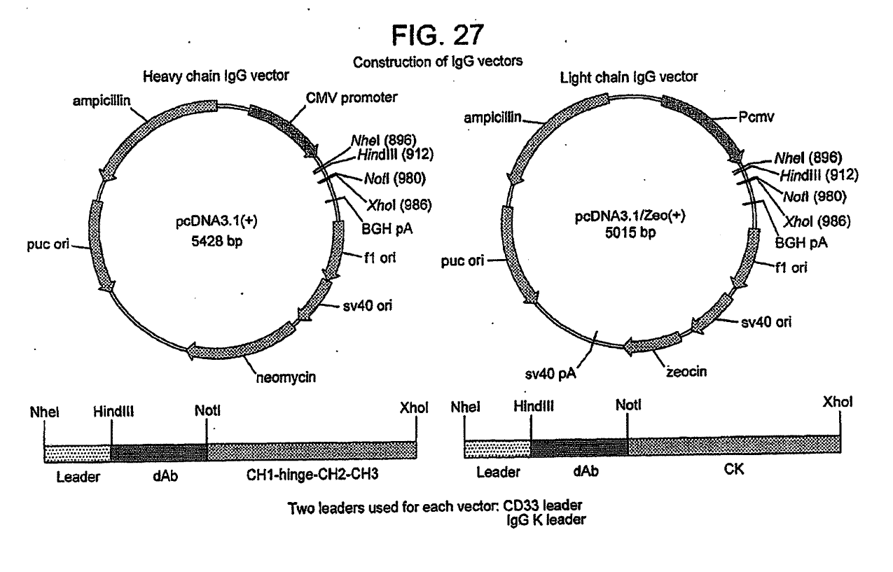

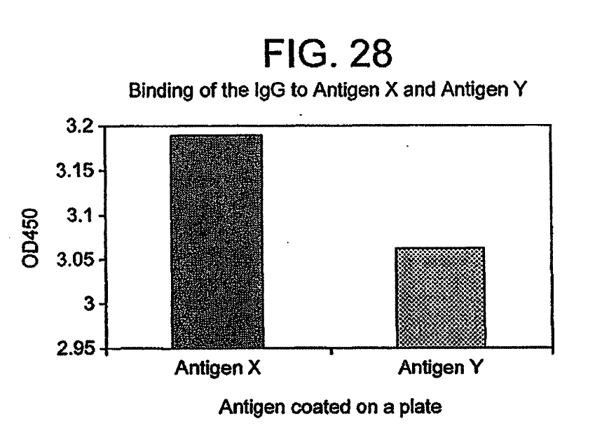

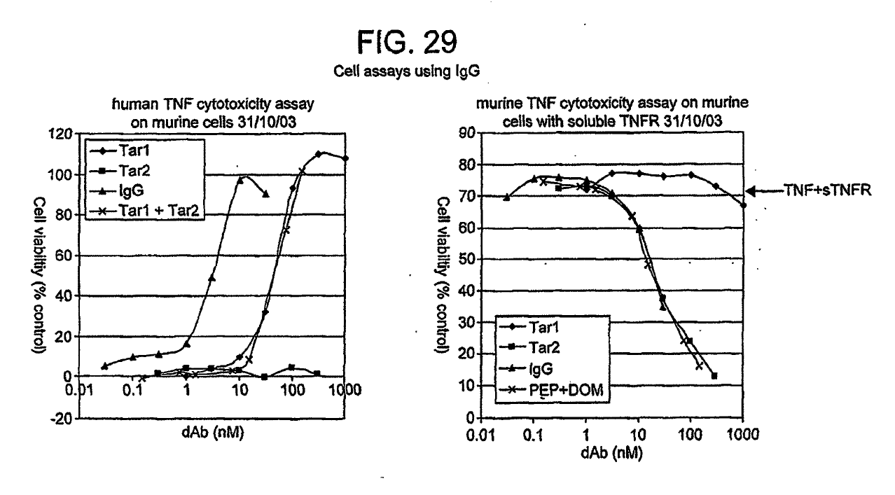

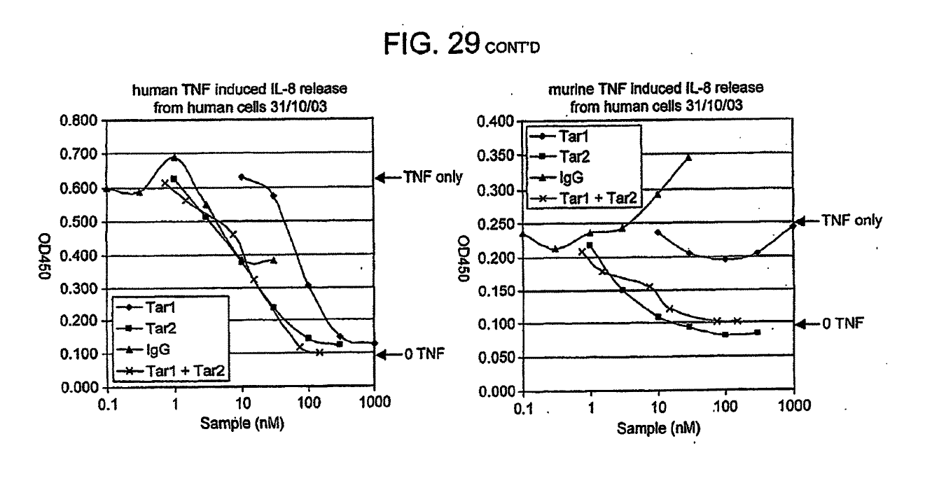

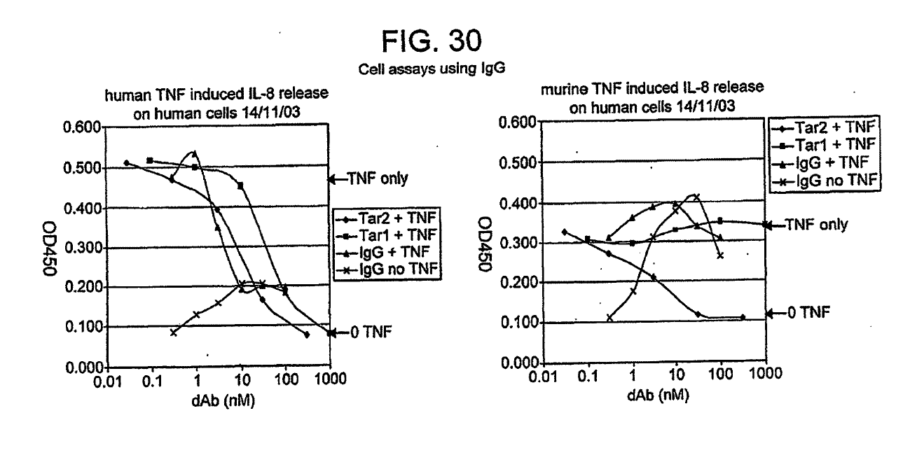

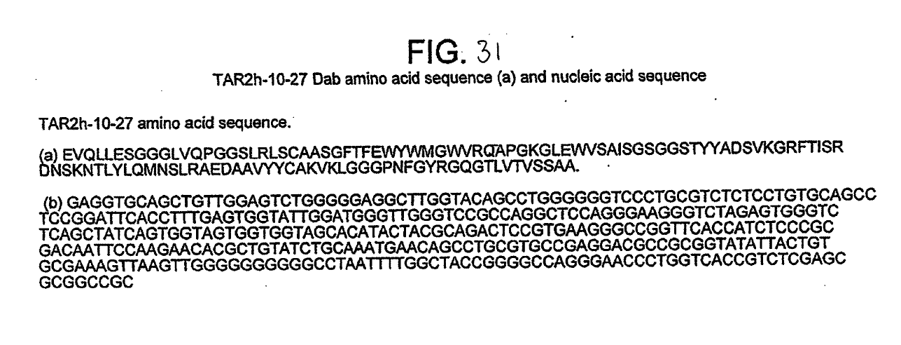

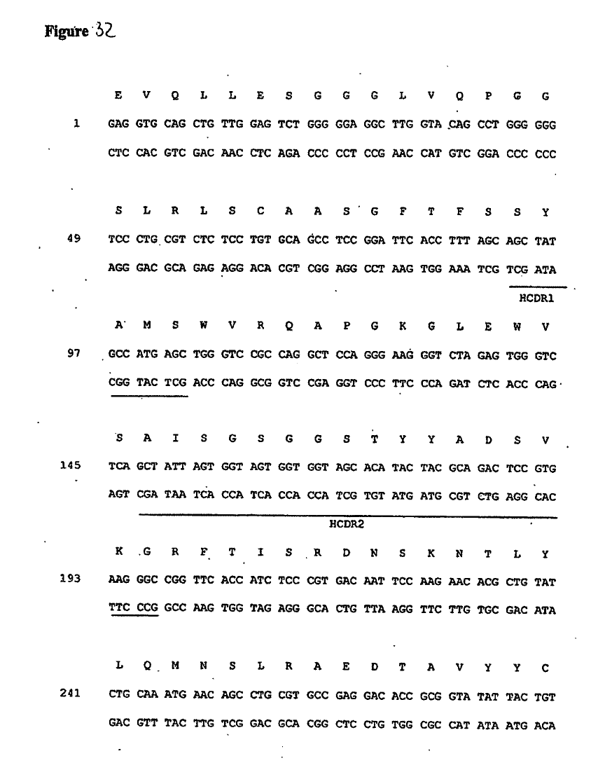

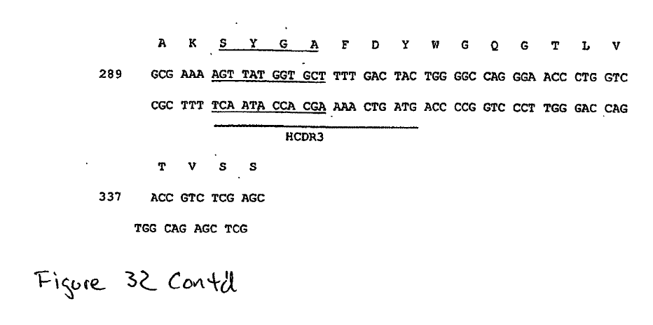

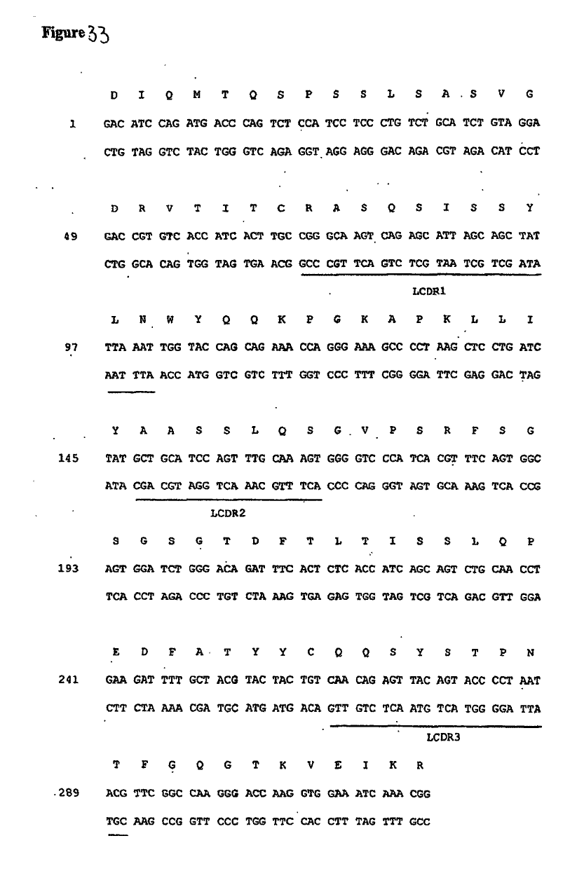

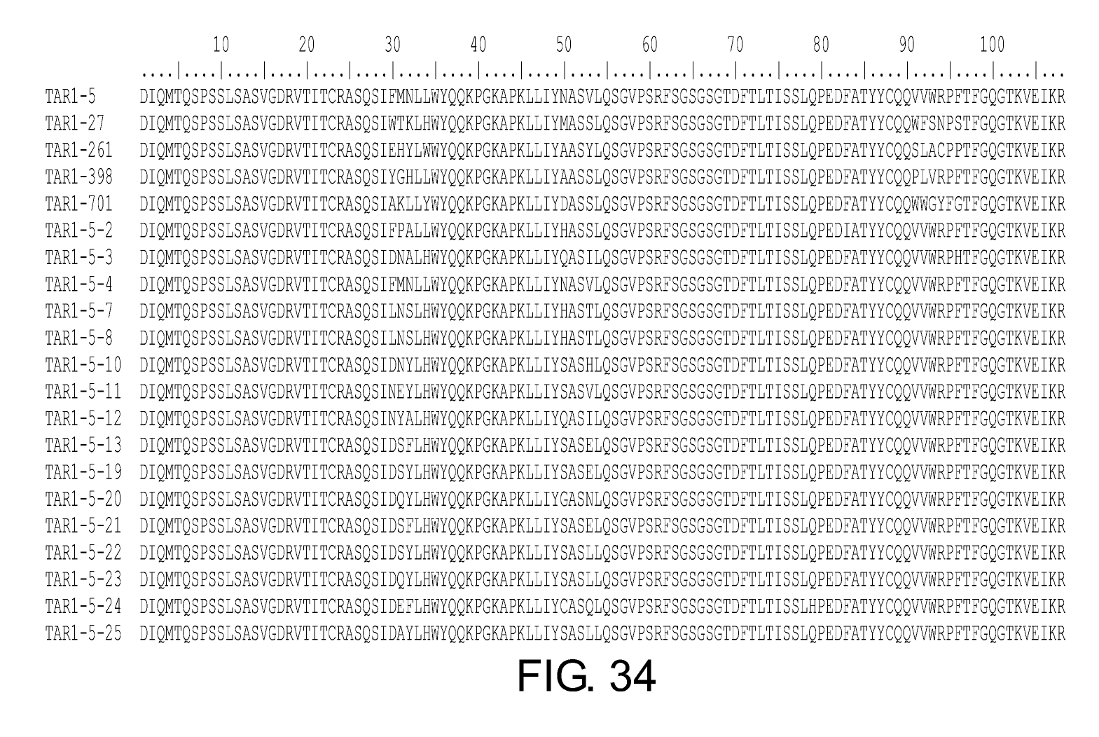

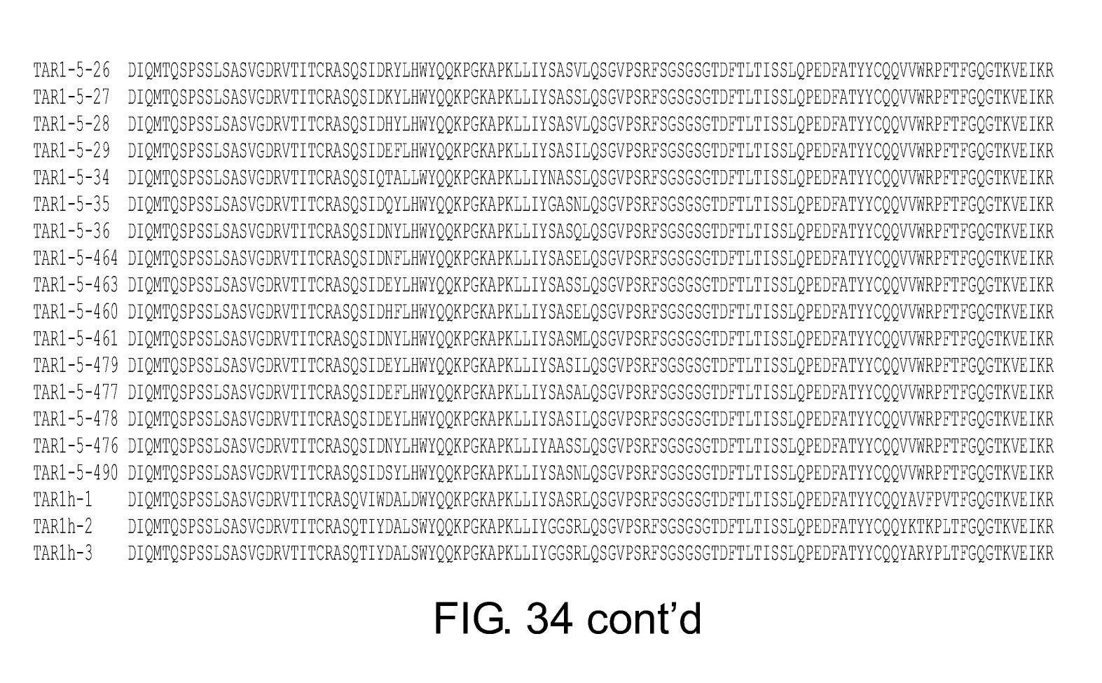

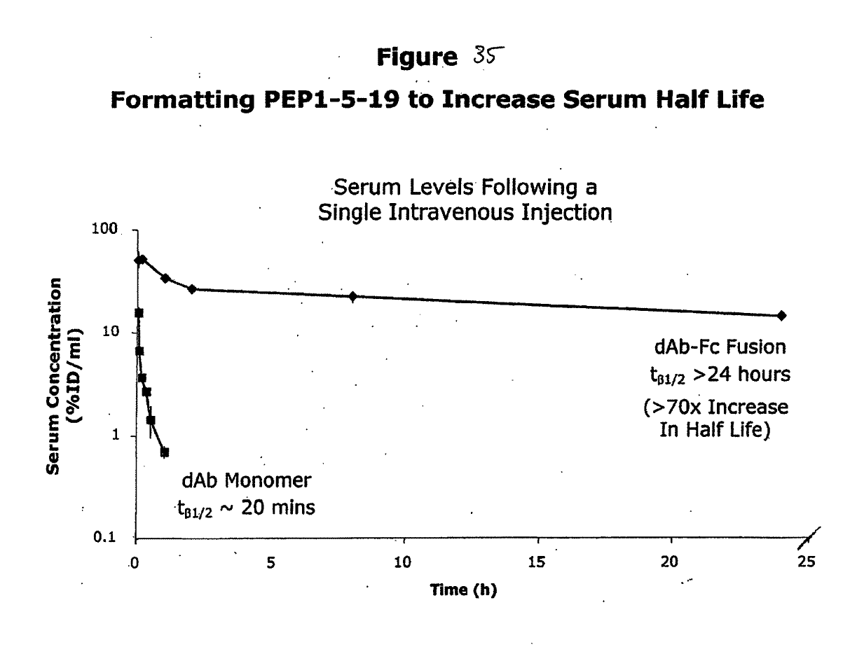

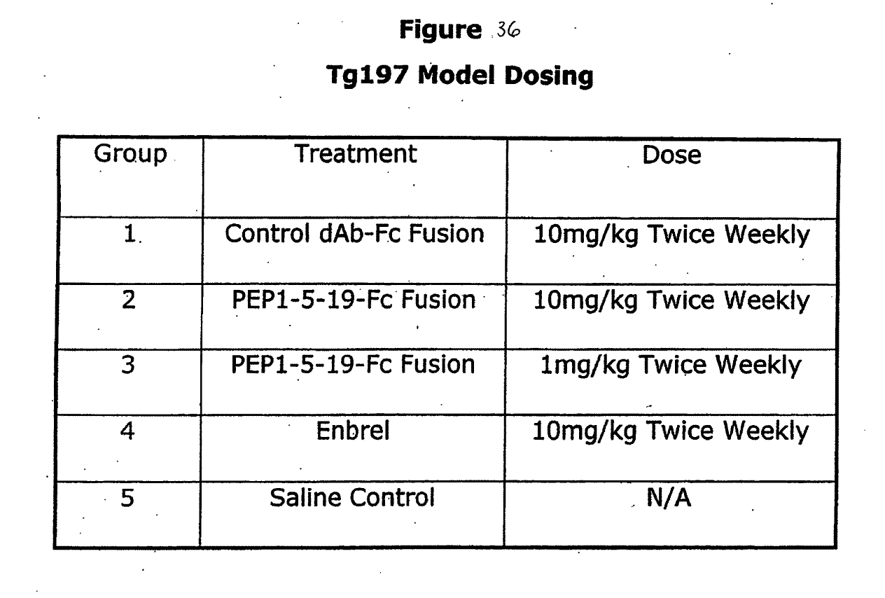

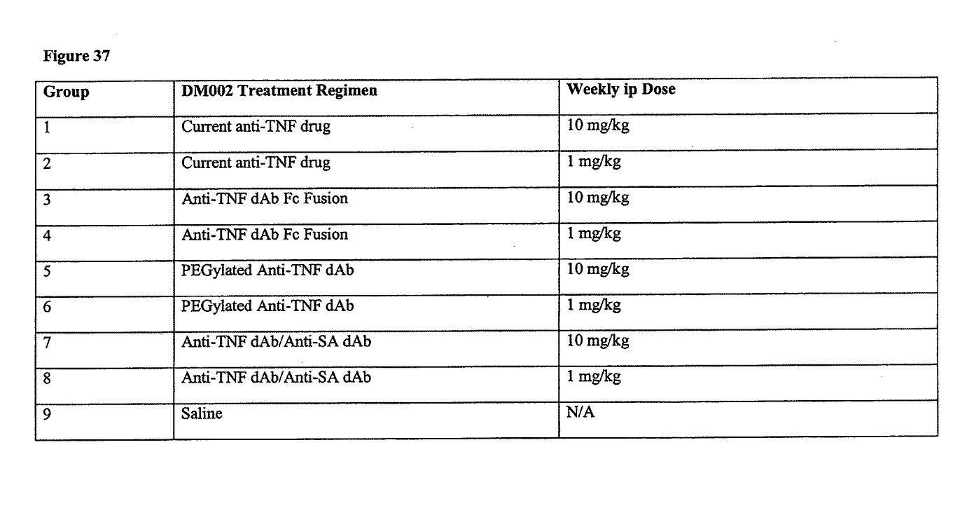

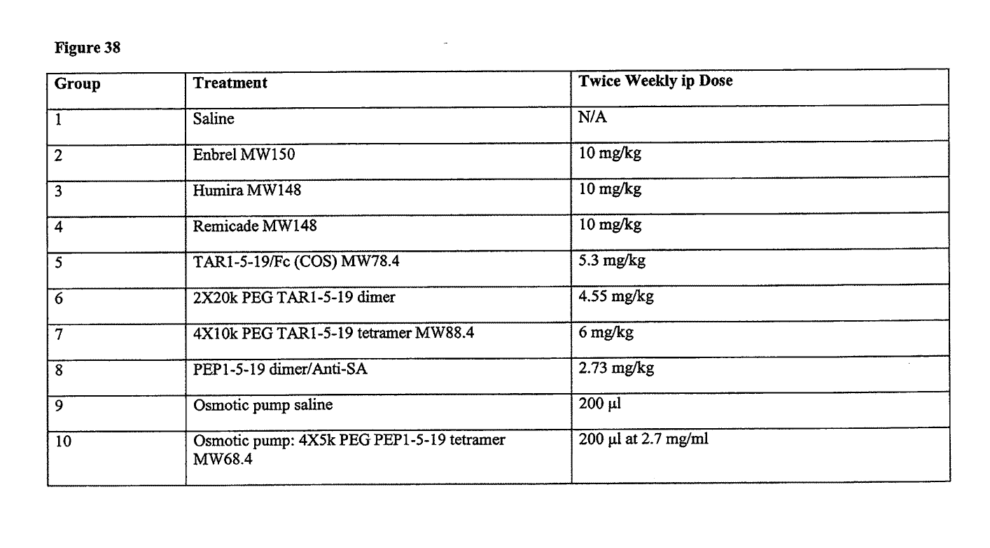

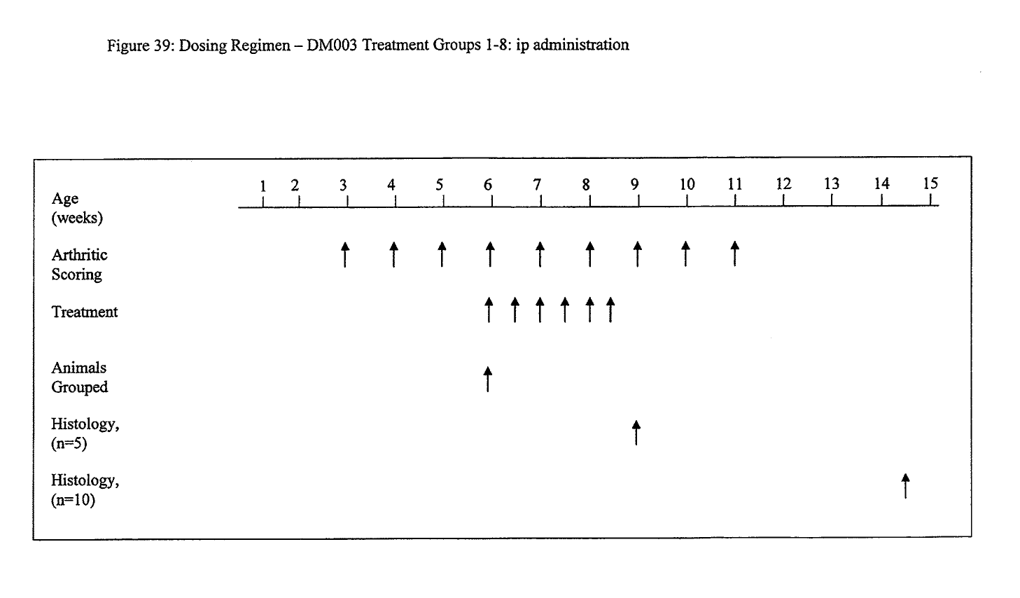

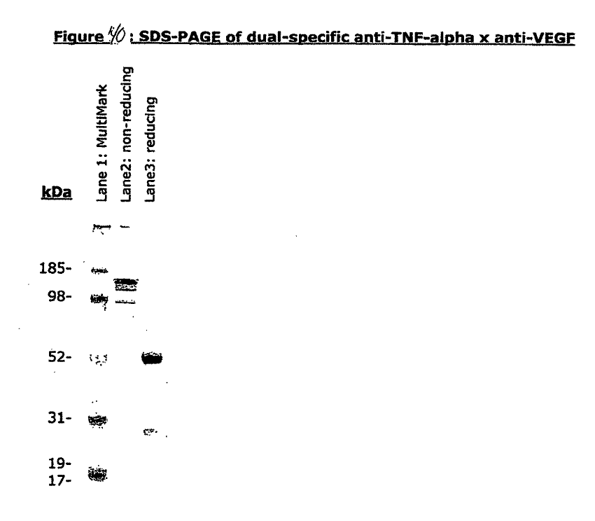

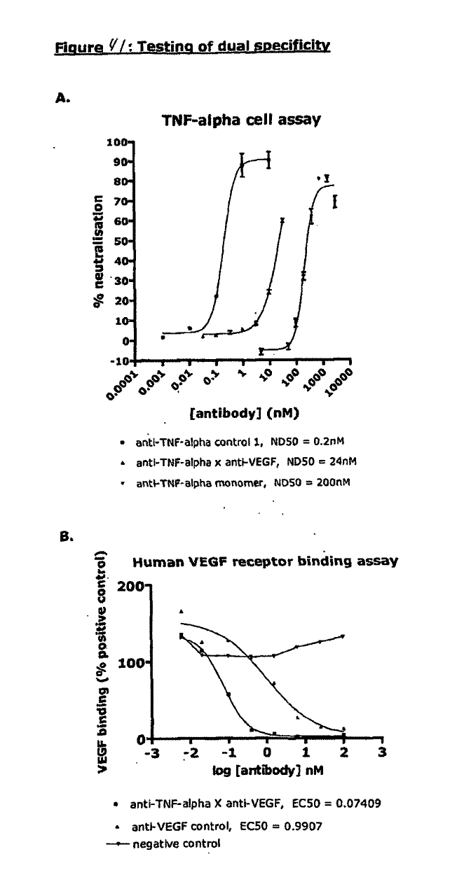

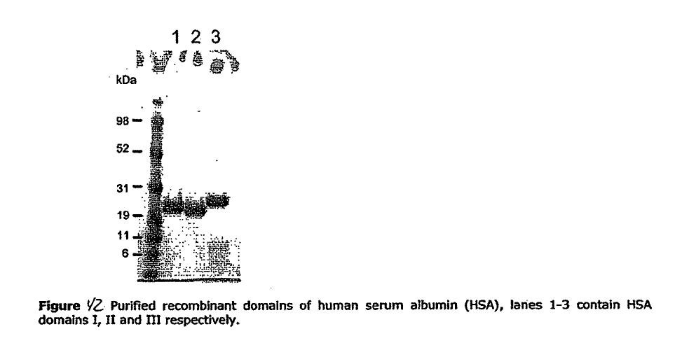

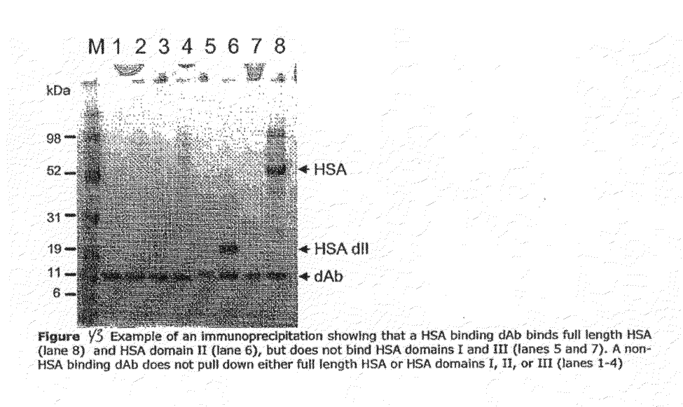

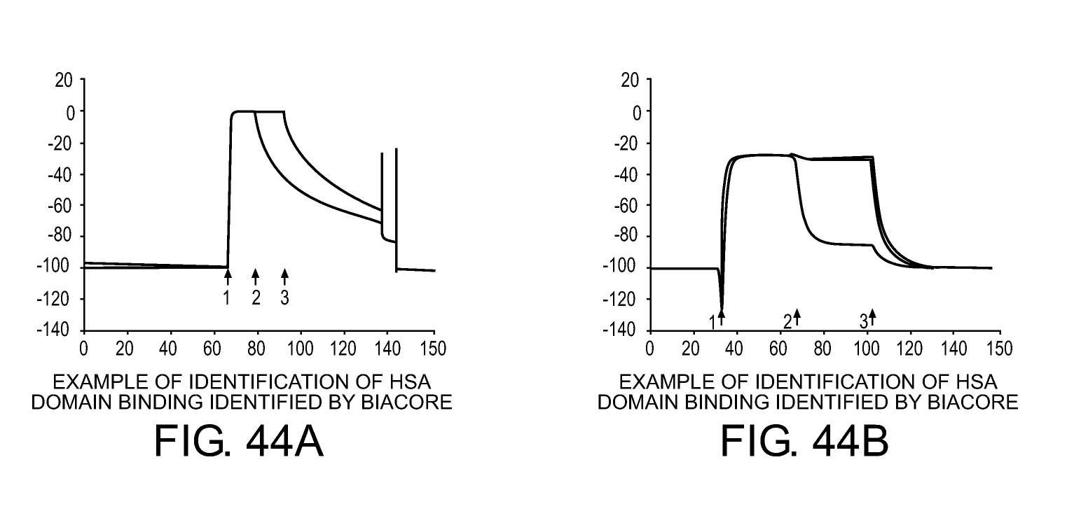

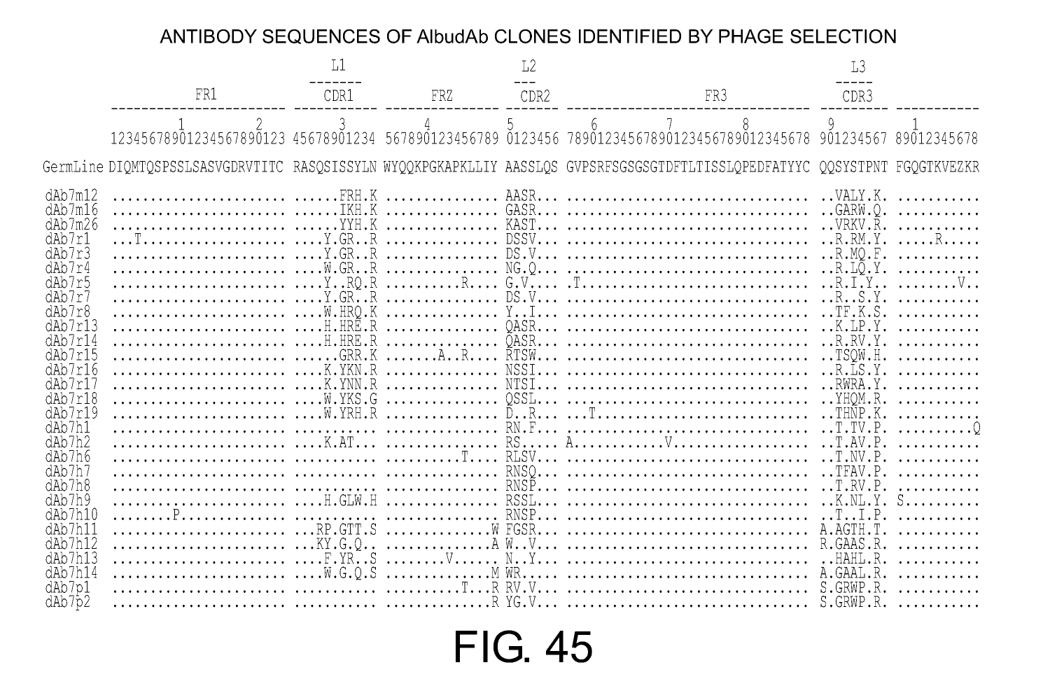

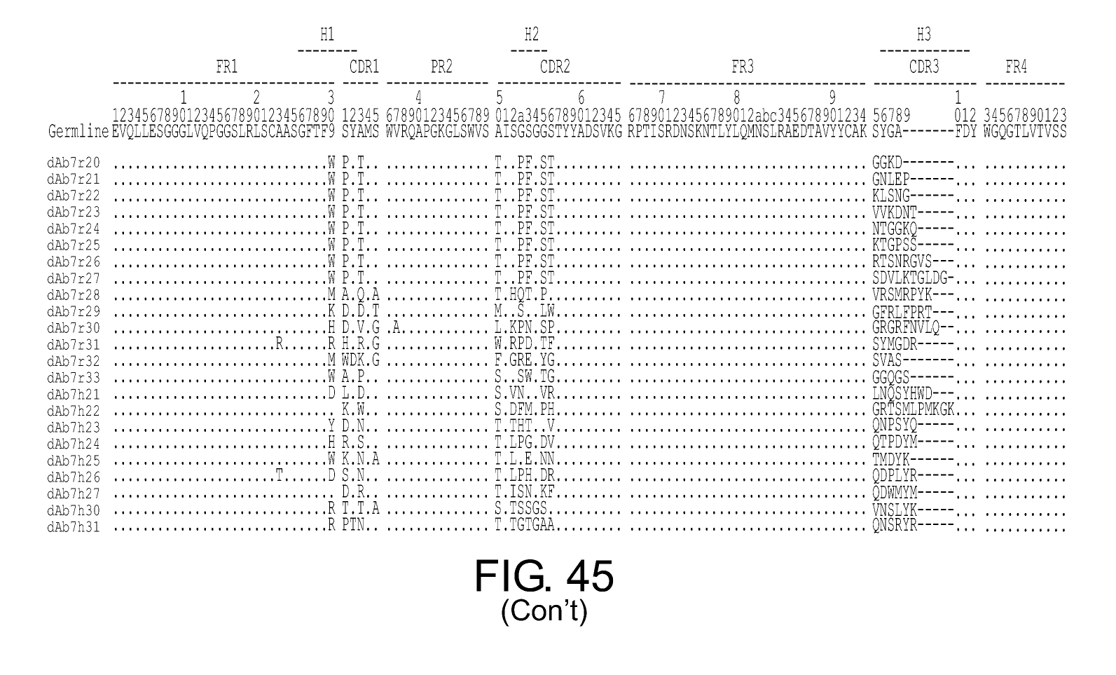

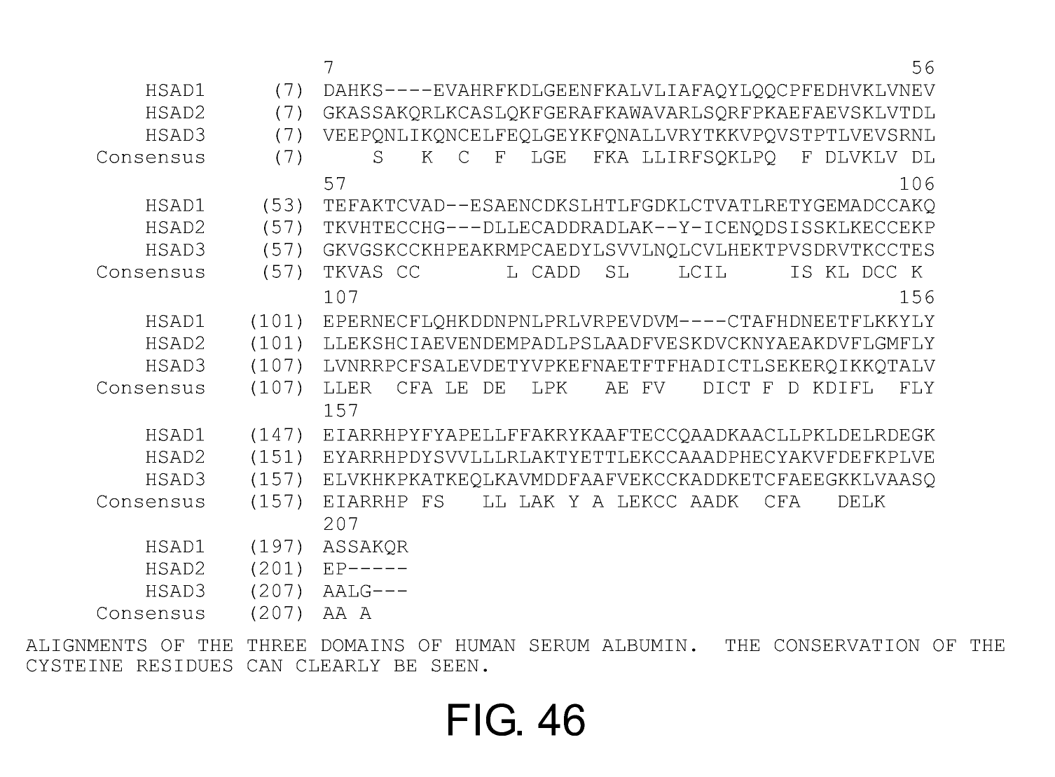

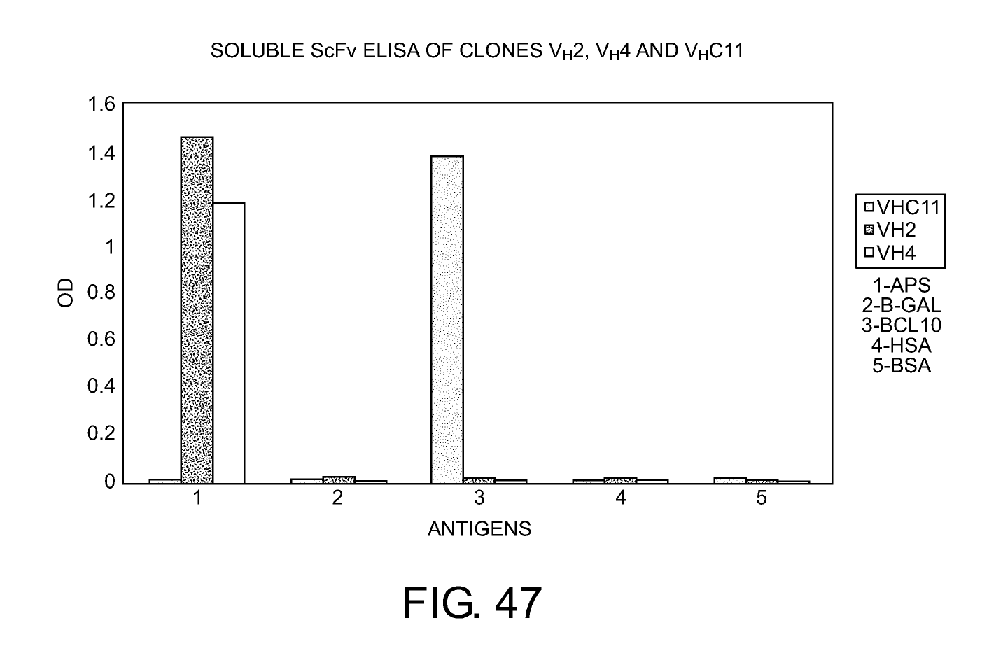

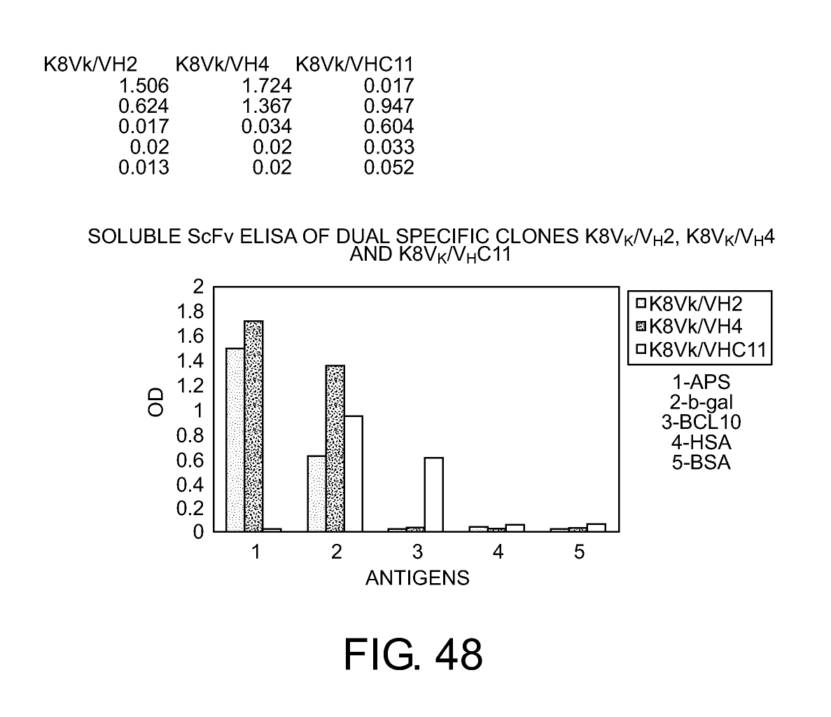

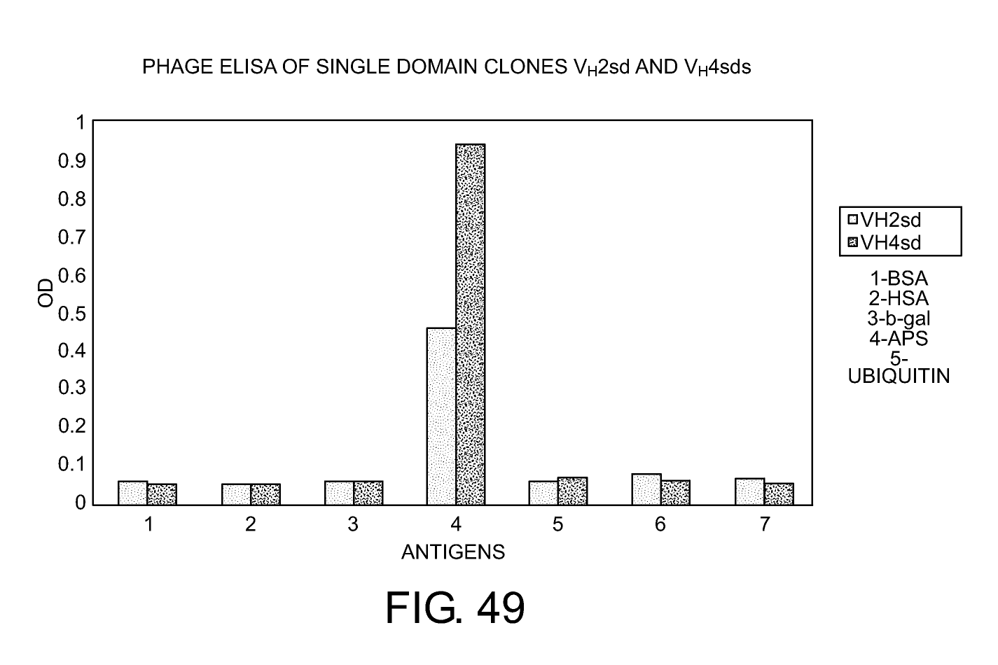

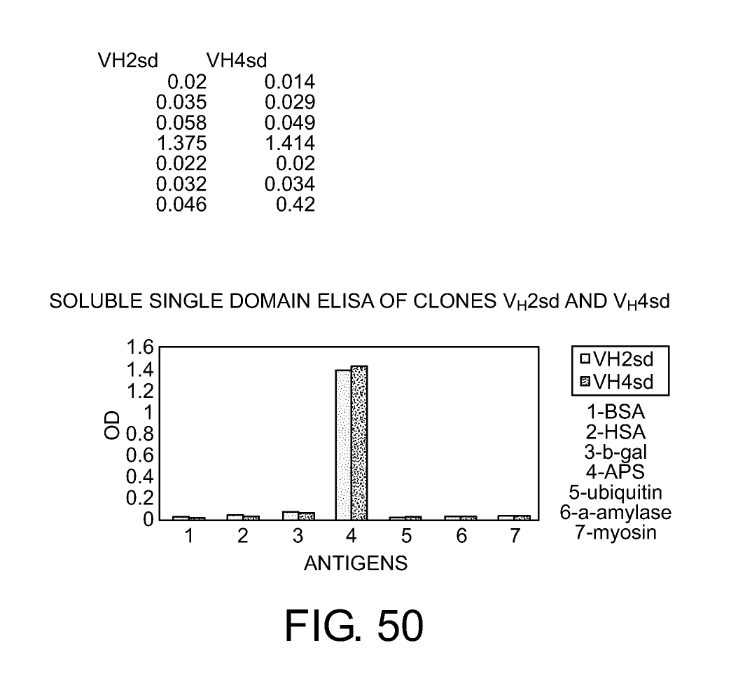

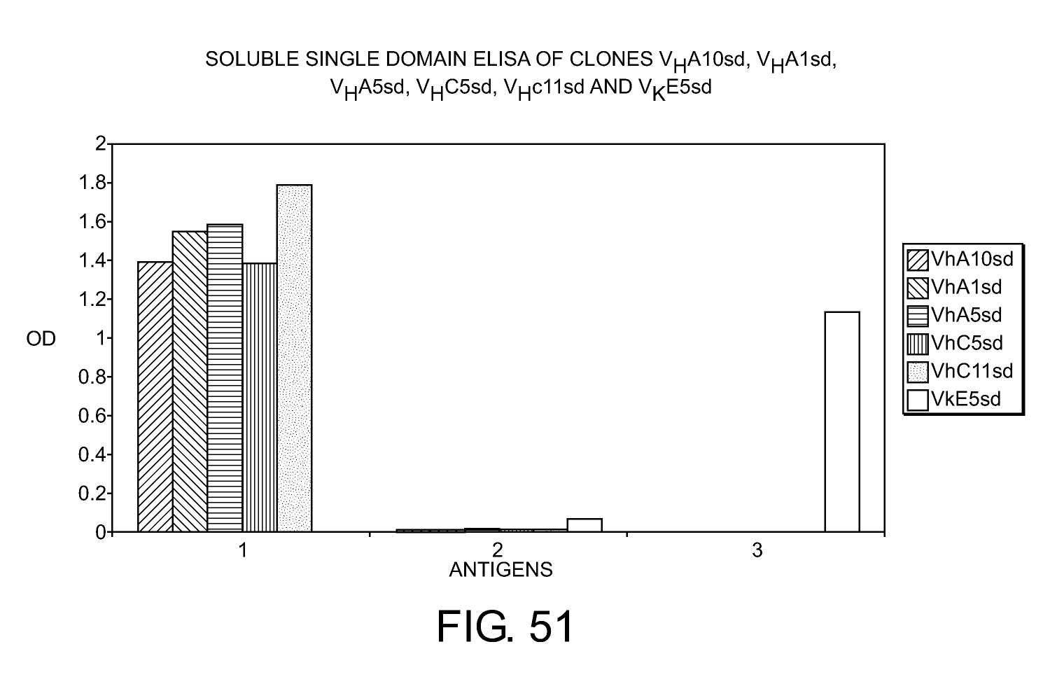

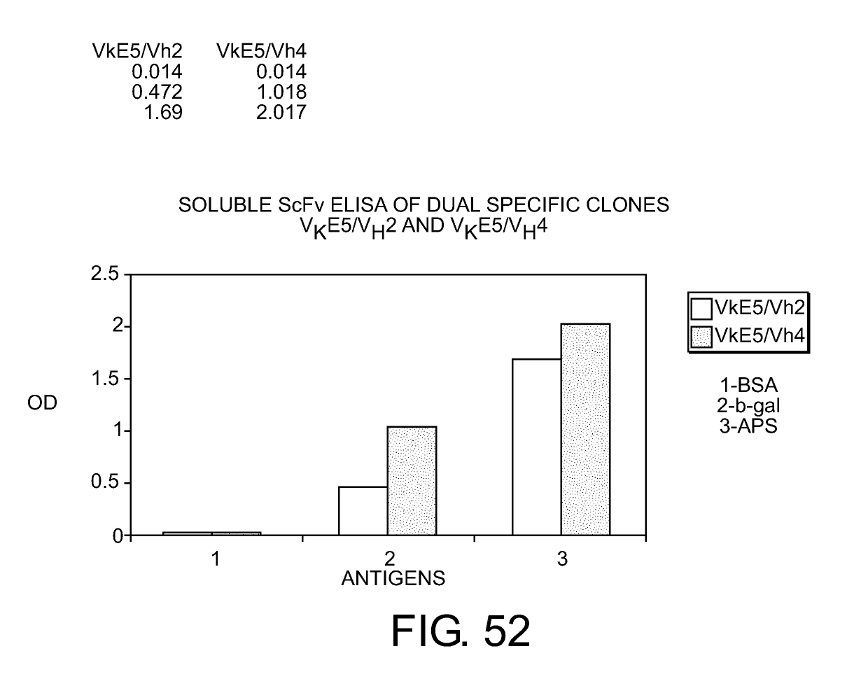

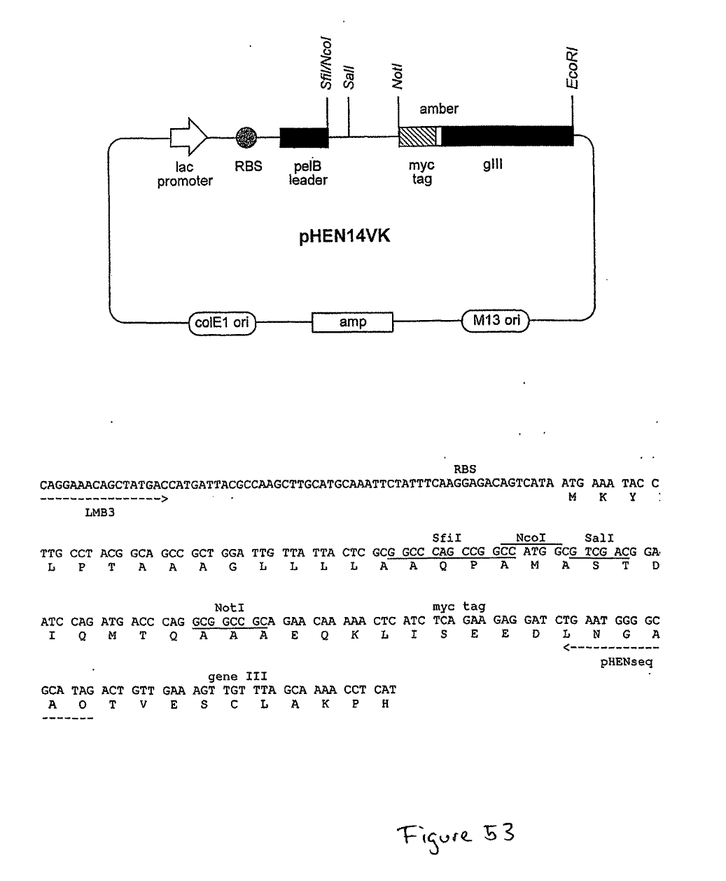

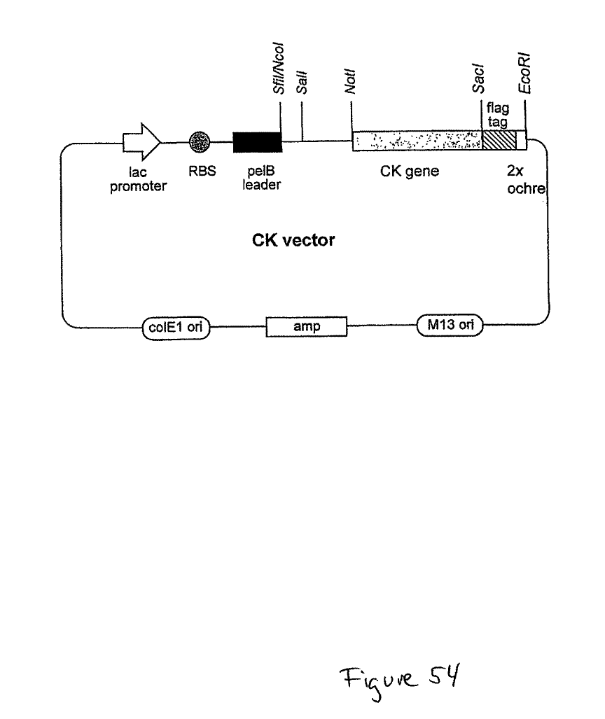

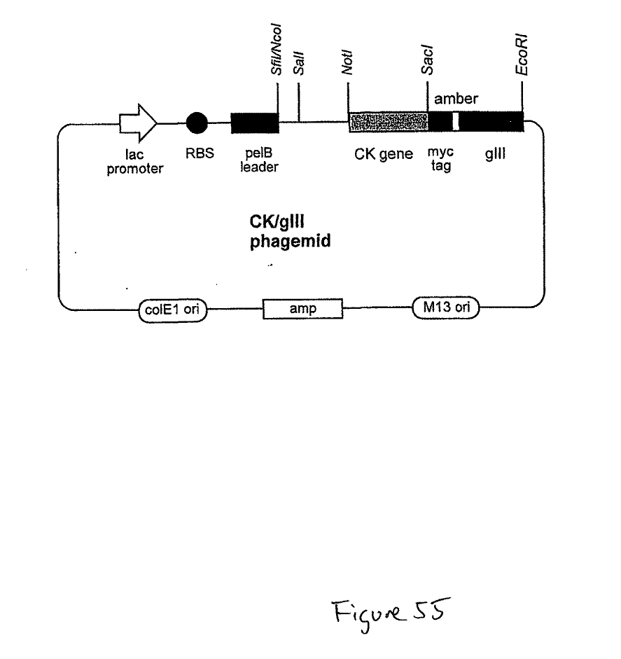

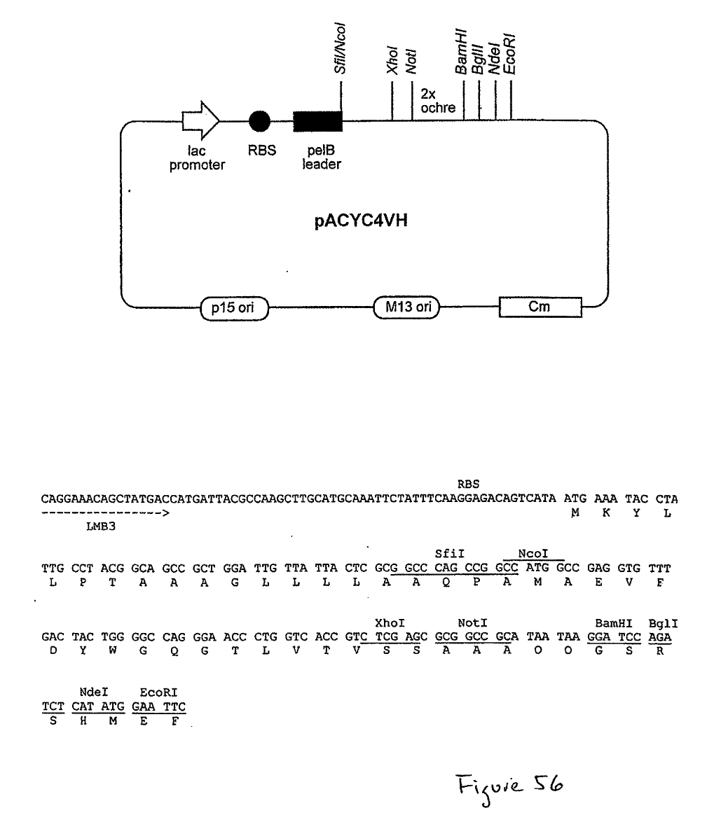

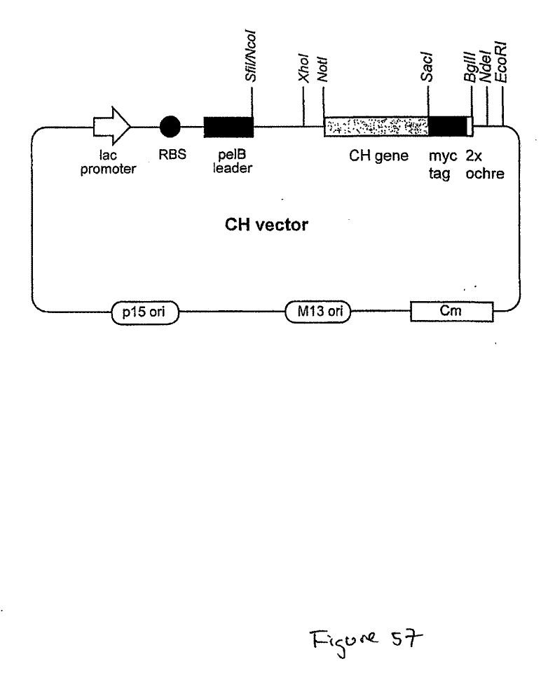

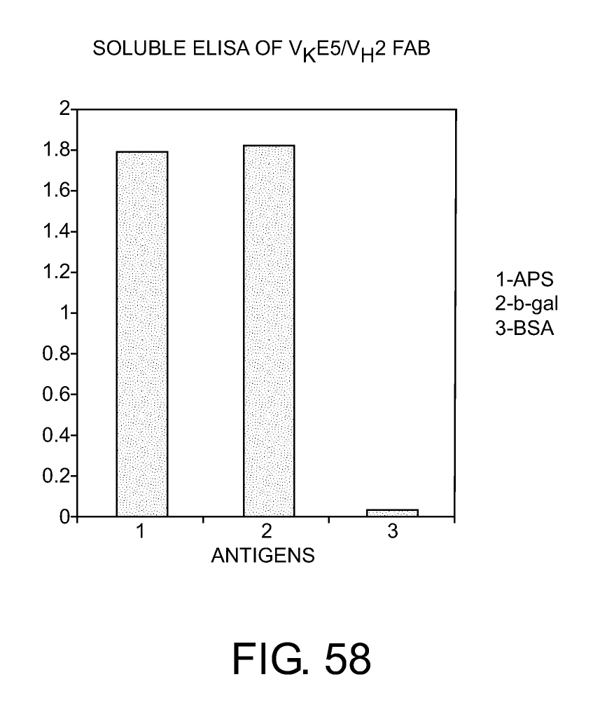

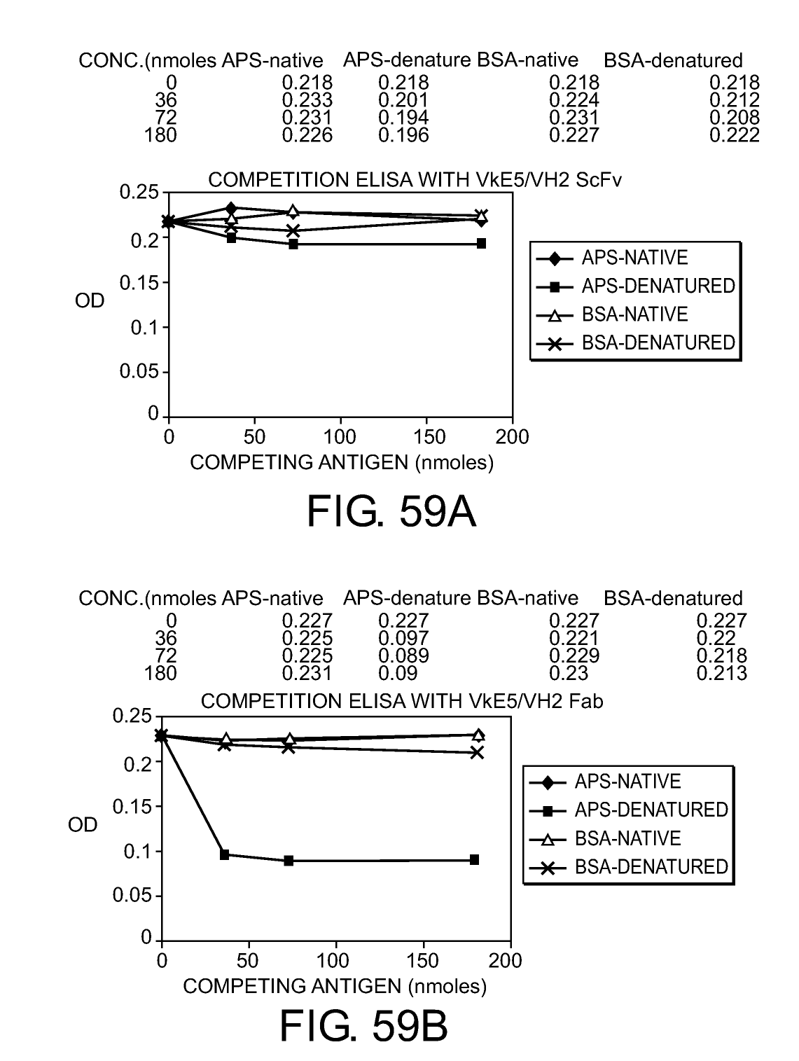

FIG. 32 Shows the polynucleotide and amino acid sequences of human germline framework segment DP47 (see also FIG. 1). Amino acid sequence is SEQ ID NO: 1; polynucleotide sequence of top strand is SEQ ID NO: 2.

FIG. 33 Shows the polynucleotide and amino acid sequences of human germline framework segment DPK9. Amino acid sequence is SEQ ID NO: 3; polynucleotide sequence of top strand is SEQ ID NO: 4.

FIG. 34 Shows amino acid sequences for the TAR1 clones described herein (see, e.g., Example 13). TAR1-5, SEQ ID NO: 241; TAR1-27, SEQ ID NO: 242; TAR1-261, SEQ ID NO: 243; TAR1-398, SEQ ID NO: 244; TAR1-701, SEQ ID NO: 245; TAR1-5-2, SEQ ID NO: 246; TAR1-5-3, SEQ ID NO: 247; TAR1-5-4, SEQ ID NO: 248; TAR1-5-7, SEQ ID NO: 249; TAR1-5-8, SEQ ID NO: 250; TAR1-5-10, SEQ ID NO: 251; TAR1-5-11, SEQ ID NO: 252; TAR1-5-12, SEQ ID NO: 253; TAR1-5-13, SEQ ID NO: 254; TAR1-5-19, SEQ ID NO: 191; TAR1-5-20, SEQ ID NO: 255; TAR1-5-21, SEQ ID NO: 256; TAR1-5-22, SEQ ID NO: 257; TAR1-5-23, SEQ ID NO: 258; TAR1-5-24, SEQ ID NO: 259; TAR1-5-25, SEQ ID NO: 260; TAR1-5-26, SEQ ID NO: 261; TAR1-5-27, SEQ ID NO: 262; TAR1-5-28, SEQ ID NO: 263; TAR1-5-29, SEQ ID NO: 264; TAR1-5-34, SEQ ID NO: 265; TAR1-5-35, SEQ ID NO: 266; TAR1-5-36, SEQ ID NO: 267; TAR1-5-464, SEQ ID NO: 268; TAR1-5-463, SEQ ID NO: 269; TAR1-5-460, SEQ IDNO: SEQ ID NO: 273; TAR1-5-478, SEQ ID NO: 274; TAR1-5-476, SEQ ID NO: 275; TAR1-5-490, SEQ ID NO: 276; TAR1h-1, SEQ ID NO: 277; TAR1h-2, SEQ ID NO: 278; TAR1h-3, SEQ ID NO: 279.

FIG. 35 Shows a comparison of serum half lives of TAR1-5-19 in either dAb monomer format or Fc fusion format following a single intravenous injection.

FIG. 36 Summarizes the dosages and timing of dAb constructs administered in a series of Tg197 model trials using TAR1-5-19.

FIG. 37 Summarizes the weekly dosages of differing formats of the TAR1-5-19 dAb (Fc fusion, PEGylated, Anti-TNF/Anti-SA dual specific) used in studies in the Tg197 mouse RA model.

FIG. 38 Summarizes the format (Fc fusion, PEG dimer, PEG tetramer, Anti-TNF/Anti-SA dual specific), delivery mode and dosage of anti-TNF dAb construct administered in a Tg197 mouse RA model study comparing the efficacy of the anti-TNF dAb constructs to the efficacy of the current anti-TNF products.

FIG. 39 Shows the dosing and scoring regimen for a study examining the efficacy of anti-TNF dAbs against established disease symptoms in the Tg197 mouse RA model.

FIG. 40 Shows an SDS PAGE gel analysis for an IgG-like dual specific antibody comprising a V_{κ} variable domain specific for human VEGF fused to human IgG1 constant domain and a V_{κ} variable domain specific for human TNF-α fused to human C_{κ} constant domain. Lane 1: InVitrogen Multimark MW markers. Lane 2: anti-TNF x anti-VEGF dual specific antibody in 1× non-reducing loading buffer. Lane 3: anti-TNF x anti-VEGF dual specific antibody in 1× loading buffer with 10 mM β-mercaptoethanol.

FIG. 41, A and B. Shows the results of assays examining the inhibitory effects of anti-TNFα anti-VEGF dual specific antibody in assays of TNF-α activity and VEGF receptor binding. A. Results of L929 TNF-α cytotoxicity neutralization assays. Curve showing data points as squares, control anti-TNF-α antibody. Curve showing data points as upward-pointing triangles, anti-TNFα anti-VEGF dual specific antibody. Curve showing data points as downward-pointing triangles, anti-TNF-α monomer. B. Results of human VEGF Receptor 2 binding assays. Curve showing data points as squares, anti-TNFα anti-VEGF dual specific antibody. Curve showing data points as upward-pointing triangles, anti-VEGF control. Curve showing data points as downward-pointing triangles, negative control.

FIG. 42 Purified recombinant domains of human serum albumin (HSA), lanes 1-3 contain HSA domains I, II and III, respectively.

FIG. 43 Example of an immunoprecipitation showing that an HSA-binding dAb binds full length HSA (lane 8) and HSA domain II (lane 6), but does not bind HSA domains I and III (lanes 5 and 7, respectively). A non-HSA-binding dAb does not pull down either full length HSA or HSA domains I, II, or III (lanes 1-4).

FIG. 44. Example of identification of HSA domain binding by a dAb as identified by surface plasmon resonance. The dAb under study was injected as described onto a low density coated human serum albumin CM5 sensor chip (Biacore). At point 1, the dAb under study was injected alone at 104. At point 2, using the co-inject command, sample injection was switched to a mixture of 1 μM dAb plus 7 μM HSA domain 1, 2 or 3, produced in Pichia. At point 3, sample injection was stopped, and buffer flow continued. Results for two different dAbs are shown in 23a) and 23b). When the dAb is injected with the HSA domain that it binds, it forms a complex that can no longer bind the HSA on the chip, hence the Biacore signal drops at point 2, with an off-rate that reflects the 3-way equilibrium between dAb, soluble HSA domain, and chip bound HSA. When the domain does not bind the dAb, the signal remains unchanged at point 2, and starts to drop only at point 3, where flow is switched to buffer. In both these cases, the dAb binds HSA domain 2.

FIG. 45 Antibody sequences of AlbudAb™ (a dAb which specifically binds serum albumin) clones identified by phage selection. All clones have been aligned to the human germ line genes. Residues that are identical to germ line have been represented by ‘.’. In the VH CDR3, the symbol ‘-’ has been used to facilitate alignment but does not represent a residue. All clones were selected from libraries based on a single human framework comprising the heavy-chain germ line genes V3-23/DP47 and JH4b for the VH libraries and the x light chain genes O12/O2/DPK9 and Jκ1 for the Vκ libraries with side chain diversity incorporated at positions in the antigen binding site.

FIG. 46 Alignments of the three domains of human serum albumin. The conservation of the cysteine residues can clearly be seen.

FIG. 47 shows the binding of dual specific scFv antibodies directed against APS and β-gal and a dual specific scFv antibody directed against BCL10 protein and β-gal to their respective antigen.

FIG. 48 shows the binding characteristics of K8V_{K}/V_{H}2/K8V_{K}/V_{H}4 and K8V_{K}/V_{H}C11 using a soluble scFv ELISA as described herein. All clones were dual specific without any cross-reactivity with other proteins.

FIG. 49 shows the binding characteristics of produced clones V_{H}2sd and V_{H}4sd tested by monoclonal phage ELISA. Phage particles were produced as described by Harrison et al in 1996. 96-well ELISA plates were coated with100 μg/ml of APS, BSA, HSA, ubiquitin, α-amylase and myosin at 10 μg/ml concentration in PBS overnight at 4° C. A standard ELISA protocol was followed using detection of bound phage with anti M13-HRP conjugate. ELISA results demonstrated that V_{H} single domains specifically recognised APS when displayed on the surface of the filamentous bacteriophage.

FIG. 50 shows the ELISA of soluble V_{H}2sd and V_{H}4sd. The same results are obtained as with the phage ELISA shown in FIG. 49, indicating that these single domains are also able to recognise APS or soluble fragments.

FIG. 51 shows the selection of single V_{H} domain antibodies directed against APS and single V_{K} domain antibodies directed against β-gal from a repertoire of single antibody domains. Soluble single domain ELISA was performed as soluble scFv ELISA described in Example 1 and bound V_{K} and V_{H} single domains were detected with Protein L-HRP and Protein A-HRP respectively. Five V_{H} single domains V_{H}A10sd, V_{H}A1sd,V_{H}A5sd, V_{H}C5sd and V_{H}C11sd selected from library 5 were found to bind APS and one V_{K} single domain V_{K}E5SD selected from library 6 was found to bind β-gal. None of the clones cross-reacted with BSA.

FIG. 52 shows the characterisation of dual specific scFv antibodies VKE5/VH2 and VKE5/VH4 directed against APS and p-gal. Soluble scFv ELISA was performed as described in example 1 and the bound scFvs were detected with Protein L-HRP. Both VKE5/VH2 and VKE5/VH4 clones were found to be dual specific. No cross reactivity with BSA was detected.

FIG. 53 shows the construction of V_{K} vector and V_{K}G3 vector. V_{K}G was pc amplified from an individual clone, A4 selected from a Fab library using BK BACKNOT as a 5′back primer and CKSACFORFL as a 3′ (forward) primer. 30 cycles of PCR amplification was performed except that Pfu polymerase was used in enzyme. PCR product was digested with NotllEcoRI and ligated into a NotIEcoRI digested vector pHEN14V_{K} to create a C_{K} vector.

FIG. 54 shows the C_{K} vector referred to in FIG. 53.

FIG. 55 shows a Ck/gIII phagemid. Gene III was PCR amplified from a pIT2 vector using G3BACKSAC as a 5′ (back) primer and LMB2 as a 3′ (forward) primer. 30 cycles of PCR amplification were performed as described herein. PCR product was digested with SACI/EcoRI and ligated into a SacI/EcoRI digested C_{K} vector.

FIG. 56 shows a C_{H} vector. C_{H} gene was PCR amplified from an individual clone A4 selected from a Fab library using CHBACKNOT as a 5′ (back) primer and CHSACFOR as a 3′ (forward) primer. 30 cycles of PCR amplification were performed as described herein. PCR product was digested with a Notl/BglII and ligated into a Notl/BglII digested vector PACYC4V_{H} to create a C_{H} vector.

FIG. 57 shows the CH vector referred to in FIG. 56.

FIG. 58 shows an ELISA of V_{K} E5/VH2 Fab.

FIG. 59 shows competition ELISAs with V_{K}E5/V_{H}2 scFv and V_{K}E5/V_{H}2 Fab.

### DETAILED DESCRIPTION OF THE INVENTION

### Definitions

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc. which are incorporated herein by reference) and chemical methods.

As used herein, the term “domain” refers to a folded protein structure which retains its tertiary structure independently of the rest of the protein. Generally, domains are responsible for discrete functional properties of proteins, and in many cases may be added, removed or transferred to other proteins without loss of function of the remainder of the protein and/or of the domain.

As used herein, a “single variable domain” is a domain which can specifically bind an epitope, an antigen or a ligand independently, that is, without the requirement for another binding domain to co-operatively bind the epitope, antigen or ligand. Such an epitope, antigen or ligand can be naturally occurring, or can be a modification of a natural occurring epitope, antigen or ligand, or can be synthetic. The “variable” portion of the single variable domain essentially determines the binding specificity of each particular single variable domain. Thus, the term “variable” in the context of single variable domains, refers to the fact that the sequence variability is not evenly distributed through a single variable domain, but is essentially distributed between the framework or skeleton portions of the single variable domain. For example, in an antibody single variable domain, the variability is concentrated in one to three segments commonly known as complementarity determining regions (CDRs). The one or more CDRs can be distributed between antibody framework regions (FR) of a light chain or of a heavy chain to form either an antibody light chain single variable domain or an antibody heavy chain single variable domain, respectively, each of which specifically binds an epitope independently of another binding domain. Similarly structured is a T-cell receptor single variable domain, with its one to three CDRs distributed between the TCR framework domains.

Thus, the variable portions conferring the binding specificity of single variable domains may differ extensively in sequence from other single variable domains having substantially the same remaining scaffold portion, and accordingly, may have a diverse range of binding specificities. Scaffolds of single variable domains include antibody framework scaffolds, consensus antibody frameworks, and scaffolds originating and/or derived from bacterial proteins, e.g. GroEL, GroEs, SpA, SpG, and from eukaryotic proteins, e.g., CTLA-4, lipocallins, fibronectin, etc. One source of the variable portions of single variable domains include one or more CDRs, which can be inserted onto non- immunoglobulin scaffolds as well as antibody framework scaffolds to generate antibody single variable domains. Another source of variation in a single variable domain can be the diversification of chosen positions in a non-immunoglobulin framework scaffold such as fibronectin, to generate single variable domains, using molecular biology techniques, such as NNK codon diversity. Similarly, this source of variation is also applicable to an antibody single variable domain.

An antibody single variable domain can be derived from antibody sequences encoded and/or generated by an antibody producing species, and includes fragment(s) and/or derivatives of the antibody variable region, including one or more framework regions, or framework consensus sequences, and/or one or more CDRs. Accordingly, an antibody single variable domain includes fragment(s) and/or derivative(s) of an antibody light chain variable region, or of an antibody heavy chain variable region, or of an antibody VHH region. For example, antibody VHH regions include those that are endogenous to camelids: e.g., camels and llamas, and the new antigen receptor (NAR) from nurse and wobbegong sharks (Roux et al., 1998). Antibody light chain variable domains and antibody heavy chain variable domains include those endogenous to an animal species including, but preferably not limited to, human, mouse, rat, porcine, cynomolgus, hamster, horse, cow, goat, dog, cat, and avian species, e.g. human VKappa and human VH3, respectively. Antibody light chain variable regions and antibody heavy chain variable regions, also includes consensus antibody frameworks, as described infra, including those of V region families, such as the VH3 family. A T-cell receptor single variable domain is a single variable domain which is derived from a T-cell receptor chain(s), e.g., α, β, γ and δ chains, and which binds an epitope or an antigen or a ligand independently of another binding domain for that epitope, antigen or ligand, analogously to antibody single variable domains.

An antibody single variable domain also encompasses a protein domain which comprises a scaffold which is not derived from an antibody or a T-cell receptor, and which has been genetically engineered to display diversity in binding specificity relative to its pre-engineered state, by incorporating into the scaffold, one or more of a CDR1, a CDR2 and/or a CDR3, derivative or fragment thereof, or an entire antibody V domain. An antibody single variable domain can also include both non-immunoglobulin scaffold and immunoglobulin scaffolds as illustrated by the GroEL single variable domain multimers described infra. Preferably the CDR(s) is from an antibody V domain of an antibody chain, e.g., VH, VL, and VHH. The antibody chain can be one which specifically binds an antigen or epitope in concert with a second antibody chain, or the antibody chain can be one which specifically binds an antigen or epitope independently of a second antibody chain, such as VHH chain. The integration of one or more CDRs into an antibody single variable domain which comprises a non-immunoglobulin scaffold must result in the non immunoglobulin scaffold's single variable domain specifically binding an epitope or an antigen or a ligand independently of another binding domain for that epitope, antigen or ligand.

A single domain is transformed into a single variable domain by introducing diversity at the site(s) designed to become the binding site, followed by selection for desired binding characteristics using, for example, display technologies. Diversity can be introduced in specific sites of a non-immunoglobulin scaffold of interest by randomizing the amino acid sequence of specific loops of the scaffold, e.g. by introducing NNK codons. This mechanism of generating diversity followed by selection of desired binding characteristics is similar to the natural selection of high affinity, antigen-specific antibodies resulting from diversity generated in the loops which make up the antibody binding site in nature. Ideally, a single domain which is small and contains a fold similar to that of an antibody loop, is transformed into a single variable domain, variants of the single variable domain are expressed, from which single variable domains containing desired binding specificities and characteristics can be selected from libraries containing a large number of variants of the single variable domain.

Nomenclature of single variable domains: sometimes the nomenclature of an antibody single variable domain is abbreviated by leaving off the first “d” or the letters “Dom”, for example, Ab7h24 is identical to dAb7h24 which is identical to DOM7h24.

By antibody single variable domain is meant a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least in part the binding activity and specificity of the full-length domain (e.g., retain a dissociation constant of 500 nM or less (e.g., 450 nM or less, 400 nM or less, 350 nM or less, 300 nM or less, 250 nM or less, 200 nM or less, 150 nM or less, 100 nM or less) and the target antigen specificity of the full-length domain). The term antibody single variable domain, as used herein, is interchangeable with the terms “single immunoglobulin variable domain” and “single domain antibody polypeptide.”

Accordingly, “single immunoglobulin variable domain” or “single domain antibody polypeptide” refers to a folded polypeptide domain which comprises sequences characteristic of immunoglobulin variable domains and which specifically binds an antigen (i.e., dissociation constant of 500 nM or less). A “single domain antibody polypeptide” therefore includes complete antibody variable domains as well as modified variable domains, for example in which one or more loops have been replaced by sequences which are not characteristic of antibody variable domains or antibody variable domains which have been truncated or comprise N- or C-terminal extensions, as well as folded fragments of variable domains which retain a dissociation constant of 500 nM or less (e.g., 450 nM or less, 400 nM or less, 350 nM or less, 300 nM or less, 250 nM or less, 200 nM or less, 150 nM or less, 100 nM or less) and the target antigen specificity of the full-length domain. Preferably an antibody single variable domain is selected from the group of V_{H} and V_{L}, including Vκ and Vλ.

The phrase “single domain antibody polypeptide construct” or “antibody single variable domain construct” encompasses not only an isolated antibody single variable domain, but also larger polypeptide constructs that comprise one or more monomers of a single immunoglobulin variable domain polypeptide sequence. It is stressed, that a single domain antibody polypeptide that is part of a larger construct is capable, on its own, of specifically binding a target antigen. Thus, a single domain antibody polypeptide construct that comprises more than one single domain antibody polypeptide does not encompass, for example, a construct in which a V_{H} and a V_{L} domain are cooperatively required to form the binding site necessary to specifically bind a single antigen molecule. The linkage between single domain antibody polypeptides in a single domain antibody polypeptide construct can be peptide or polypeptide linkers, or, alternatively, can be other chemical linkages, such as through linkage of polypeptide monomers to a multivalent PEG. The linked single domain antibody polypeptides can be identical or different, and the target specificities of the constituent polypeptides can likewise be the same or different.

Complementary: Two immunoglobulin domains are “complementary” where they belong to families of structures which form cognate pairs or groups or are derived from such families and retain this feature. For example, a VH domain and a VL domain of an antibody are complementary; two VH domains are not complementary, and two V domains are not complementary. Complementary domains may be found in other members of the immunoglobulin superfamily, such as the V_{α} and V_{β} (or γ and δ) domains of the T-cell receptor. In the context of the second configuration of the invention, non-complementary domains do not bind a target molecule cooperatively, but act independently on different target epitopes which may be on the same or different molecules. Domains which are artificial, such as domains based on protein scaffolds which do not bind epitopes unless engineered to do so, are non- complementary. Likewise, two domains based on (for example) an immunoglobulin domain and a fibronectin domain are not complementary.

Immunoglobulin: This refers to a family of polypeptides which retain the immunoglobulin fold characteristic of antibody molecules, which contains two β sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signalling (for example, receptor molecules, such as the PDGF receptor).

The present invention is applicable to all immunoglobulin superfamily molecules which possess binding domains. Preferably, the present invention relates to antibodies.

Combining: Variable domains according to the invention are combined to form a group of domains; for example, complementary domains may be combined, such as V_{L} domains being combined with VH domains. Non-complementary domains may also be combined. Domains may be combined in a number of ways, involving linkage of the domains by covalent or non-covalent means.

Closed conformation multi-specific ligand: The phrase describes a multi-specific ligand as herein defined comprising at least two epitope binding domains as herein deemed. The term ‘closed conformation’ (multi-specific ligand) means that the epitope binding domains of the ligand are arranged such that epitope binding by one epitope binding domain competes with epitope binding by another epitope binding domain. That is, cognate epitopes may be bound by each epitope binding domain individually but not simultaneously. The closed conformation of the ligand can be achieved using methods herein described.

Antibody: An antibody (for example IgG, IgM, IgA, IgD or IgE) or fragment (such as a Fab, F(ab′)2, Fv, disulphide linked Fv, scFv, closed conformation multispecific antibody, disulphide-linked scFv, diabody) whether derived from any species naturally producing an antibody, or created by recombinant DNA technology; whether isolated from serum, B-cells, hybridomas, transfectomas, yeast or bacteria).

Dual-specific ligand: A ligand comprising a first immunoglobulin single variable domain and a second immunoglobulin single variable domain as herein defined, wherein the variable regions are capable of binding to two different antigens or two epitopes on the same antigen which are not normally bound by a monospecific immunoglobulin. For example, the two epitopes may be on the same hapten, but are not the same epitope or sufficiently adjacent to be bound by a monospecific ligand. A dual specific ligand to according to the invention can be composed of mutually complementary variable domain pairs which have different specificities, and do not contain mutually complementary variable domain pairs which have the same specificity. The dual specific ligands according to the invention are composed of variable domains which have different specificities, and do not contain mutually complementary variable domain pairs which have the same specificity. Thus, dual specific ligands, which as defined herein contain two single variable domains, are a subset of multimeric ligands, which as defined herein contain two or more single variable domains, wherein at least two of the single variable domains are capable of binding to two different antigens or to two different epitopes on the same antigen.

Further, a dual specific ligand can also be defined as distinct from a ligand comprising an antibody single variable domain, and a second antigen and/or epitope binding domain which is not a single variable domain. Further still, a dual specific ligand as defined herein is also distinct form a ligand containing a first and a second antigen/epitope binding domain, where neither antigen/epitope binding domain is a single variable domain as defined herein.

Antigen: A molecule that is bound by a ligand according to the present invention. Typically, antigens are bound by antibody ligands and are capable of raising an antibody response in vivo. It may be a polypeptide, protein, nucleic acid or other molecule. Generally, the dual specific ligands according to the invention are selected for target specificity against a particular antigen. In the case of conventional antibodies and fragments thereof, the antibody binding site defined by the variable loops (L1, L2, L3 and H1, H2, H3) is capable of binding to the antigen.

Epitope: A unit of structure conventionally bound by an immunoglobulin VH/VL pair. Epitopes define the minimum binding site for an antibody, and thus represent the target of specificity of an antibody. In the case of a single domain antibody, an epitope represents the unit of structure bound by a variable domain in isolation. An epitope binding domain comprises a protein scaffold and epitope interaction sites (which are advantageously on the surface of the protein scaffold). An epitope binding domain can comprise epitope interaction sites that are nonlinear, e.g. where the epitope binding domain comprises multiple epitope interaction sites that have intervening regions between them, e.g., CDRs separated by FRs, or are present on separate polypeptide chains. Alternatively, an epitope binding domain can comprise a linear epitope interaction site composed of contiguously encoded amino acids on one polypeptide chain.

A fragment as used herein refers to less than 100% of the sequence (e.g., up to 99%, 90%, 80%, 70%, 60%, 50%, 40%, 30%, 20%, 10% etc.), but comprising 5, 6, 7, 8, 9, 10, 12, 13, 14, 15, 16, 17, 18, 19, 20, 21, 22, 23, 24, 25 or more amino acids. A fragment is of sufficient length such that the serum albumin binding of interest is maintained with affinity of 1×10⁻⁶ M or more. A fragment as used herein also refers to optional insertions, deletions and substitutions of one or more amino acids which do not substantially alter the ability of the altered polypeptide to bind to a single domain antibody raised against the target. The number of amino acid insertions deletions or substitutions is preferably up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

Generic ligand: A ligand that binds to all members of a repertoire. Generally, not bound through the antigen binding site as defined above. Non-limiting examples include protein A, protein L and protein G.

Selecting: Derived by screening, or derived by a Darwinian selection process, in which binding interactions are made between a domain and the antigen or epitope or between an antibody and an antigen or epitope. Thus a first variable domain may be selected for binding to an antigen or epitope in the presence or in the absence of a complementary variable domain.

Universal framework: A single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, (1987) J. Mol. Biol. 196:910-917. The invention provides for the use of a single framework, or a set of such frameworks, which has been found to permit the derivation of virtually any binding specificity though variation in the hypervariable regions alone.

As used herein “conjugate” refers to a composition comprising an antigen binding fragment of an antibody that binds serum albumin that is bonded to a drug.

As used herein, the term “small molecule” means a compound having a molecular weight of less than 1,500 daltons, preferably less than 1000 daltons.

Such conjugates include “drug conjugates,” which comprise an antigen-binding fragment of an antibody that binds serum albumin to which a drug is covalently bonded, and “noncovlaent drug conjugates,” which comprise an antigen-binding fragment of an antibody that binds serum albumin to which a drug is noncovalently bonded.

As used herein, “drug conjugate” refers to a composition comprising an antigen-binding fragment of an antibody that binds serum albumin to which a drug is covalently bonded. The drug can be covalently bonded to the antigen-binding fragment directly or indirectly through a suitable linker moiety. The drug can be bonded to the antigen-binding fragment at any suitable position, such as the amino- terminus, the carboxyl-terminus or through suitable amino acid side chains (e.g., the amino group of lysine).

Homogeneous immunoassay: An immunoassay in which analyte is detected without need for a step of separating bound and un-bound reagents.

Substantially identical (or “substantially homologous”): A first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities. In the case of first and second antibodies and/or single variable domains described herein, the second antibody or single variable domain has the same binding specificity as the first and has at least 50%, or at least up to 55%, 60%, 70%, 75%, 80%, 90%, 95%, 96%, 97%, 98%, 99% or 100% of the affinity of the first antibody or single variable domain.

A “domain antibody” or “dAb” is equivalent to a “single immunoglobulin variable domain polypeptide” or a “single domain antibody polypeptide” as the term is used herein.

As used herein, the phrase “specifically binds” refers to the binding of an antigen by an immunoglobulin variable domain with a dissociation constant (K_{d}) of 1 μM or lower as measured by surface plasmon resonance analysis using, for example, a BIAcore™ surface plasmon resonance system and BIAcore™ kinetic evaluation software (e.g., version 2.1). The affinity or K_{d} for a specific binding interaction is preferably about 500 nM or lower, more preferably about 300 nM or lower.

As used herein, the term “high affinity binding” refers to binding with a K_{d} of less than or equal to 100 nM.

“Surface Plasmon Resonance” Competition assays can be used to determine if a specific antigen or epitope, such as human serum albumin, competes with another antigen or epitope, such as cynomolgus serum albumin, for binding to a serum albumin binding ligand described herein, such as a specific dAB. Similarly competition assays can be used to determine if a first ligand such as dAb, competes with a second ligand such as a dAb for binding to a target antigen or epitope. The term “competes” as used herein refers to substance, such as a molecule, compound, preferably a protein, which is able to interfere to any extent with the specific binding interaction between two or more molecules. The phrase “does not competitively inhibit” means that substance, such as a molecule, compound, preferably a protein, does not interfere to any measurable or significant extent with the specific binding interaction between two or more molecules. The specific binding interaction between two or more molecules preferably includes the specific binding interaction between a single variable domain and its cognate partner or target. The interfering or competing molecule can be another single variable domain or it can be a molecule that that is structurally and/or functionally similar to a cognate partner or target.

In vitro competition assays for determining the ability of a single variable domain to compete for binding to a target to another target binding domain, such as another single variable domain, as well as for determining the Kd, are well know in the art. One preferred competition assay is a surface plasmon resonance assay, which has the advantages of being fast, sensitive and useful over a wide range of protein concentrations, and requiring small amounts of sample material. A preferred surface plasmon resonance assay competition is a competition biacore experiment. A competition biacore experiment can be used to determine whether, for example, cynomolgus serum albumin and human serum albumin compete for binding to a ligand such as dAb DOM7h-x. One experimental protocol for such an example is as follows.

For example, after coating a CM5 sensor chip (Biacore AB) at 25° C. with approximately 1000 resonance units (RUs) of human serum albumin (HSA), a purified dAb is injected over the antigen surface at a single concentration (e.g., 1 um) alone, and in combination with a dilution series of the cynomolgus serum albumin (CSA). The serial dilutions of HSA were mixed with a constant concentration (40 nM) of the purified dAb. A suitable dilution series of CSA would be starting at 5 uM CSA, with six two-fold dilutions down to 78 nM CSA. These solutions must be allowed to reach equilibrium before injection. Following the injection, a response reading was taken to measure the resulting binding RUs for the dAb alone and each of the several dAb/CSA mixtures, the data being used in accordance with BIA evaluation software, generate a dose-response curve for each CSA's inhibition of the AlbudAb™'s (a dAb which specifically binds serum albumin) binding to the chip on which HSA is immobilized. By comparing the bound RUs of dAb alone with the bound RUs of dAb+CSA, one will be able to see whether the CSA competes with the HSA to bind the dAb. If it does compete, then as the CSA concentration in solution is increased, the RUs of dAb bound to HSA will decrease. If there is no competition, then adding CSA will have no impact on how much dAb binds to HSA.

One of skill would know how to adapt this or other protocols in order to perform this competition assay on a variety of different ligands, including the several ligands described herein that bind serum albumin. The variety of ligands includes, but is not limited to, monomer single variable domains, including single variable domains comprising an immunoglobulin and/or a non-immunoglobulin scaffold, dAbs, dual specific ligands, and multimers of these ligands. One of skill would also know how to adapt this protocol in order to compare the binding of several different pairs of antigens and/or epitopes to a ligand using this competition assay.

These competition experiments can provide a numeric cut-off by which one can determine if an antigen or epitope competes with another antigen or epitope for binding to a specific ligand, preferably a dAb. For example, in the experiment outlined above, if 5 uM CSA in solution results in a 10%, or lower, reduction in RUs of dAb binding to HSA, then there is considered to be no competition for binding. Accordingly, a reduction in RUs of dAb binding to HSA in the presence of CSA of greater than 10% would indicate the presence of competition for binding of the dAb for binding HSA by CSA. A reduction in RUs of dAb binding to HSA of less than 10% would indicate the absence of competition by CSA for the dAb's binding HSA, with reductions of 9%, 8%, 7%, 6%, 5%, 4%, 3%, 2%, and 1% being progressively more stringent requirements for indicating the absence of competition. The greater the reduction in RUs of dAb binding to HSA, the greater the competition. Thus, increasing levels of competition can be graded according to the percent reduction in RUs binding to HSA, i.e. at least 15%, 20%, 25%, 30%, 35%, 40%, 45%, 50%, 55%, 60%, 65%, 70%, 75%, 80%, 85%, 90%, 95%, or up to 100% reduction.

As used herein, the phrase “human single domain antibody polypeptide” refers to a polypeptide having a sequence derived from a human germline immunoglobulin V region. A sequence is “derived from a human germline V region” when the sequence is either isolated from a human individual, isolated from a library of cloned human antibody gene sequences (or a library of human antibody V region gene sequences), or when a cloned human germline V region sequence was used to generate one or more diversified sequences (by random or targeted mutagenesis) that were then selected for binding to a desired target antigen. At a minimum, a human immunoglobulin variable domain has at least 85% amino acid similarity (including, for example, 87%, 90%, 93%, 95%, 97%, 99% or higher similarity) to a naturally-occurring human immunoglobulin variable domain sequence.

Alternatively, or in addition, “a human immunoglobulin variable domain” is a variable domain that comprises four human immunoglobulin variable domain framework regions (W1-FW4), as framework regions are set forth by Kabat et al. (1991, supra). The “human immunoglobulin variable domain framework regions” encompass a) an amino acid sequence of a human framework region, and b) a framework region that comprises at least 8 contiguous amino acids of the amino acid sequence of a human framework region. A human immunoglobulin variable domain can comprise amino acid sequences of FW1-FW4 that are the same as the amino acid sequences of corresponding framework regions encoded by a human germline antibody gene segment, or it can also comprise a variable domain in which FW1-FW4 sequences collectively contain up to 10 amino acid sequence differences, up to 9 amino acid sequence differences, up to 8 amino acid sequence differences, up to 7 amino acid sequence differences, up to 6 amino acid sequence differences, up to 5 amino acid sequence differences, up to 4 amino acid sequence differences, up to 3 amino acid sequence differences, up to 2 amino acid sequence differences, or up to 1 amino acid sequence differences, relative to the amino acid sequences of corresponding framework regions encoded by a human germline antibody gene segment.

A “human immunoglobulin variable domain” as defined herein has the capacity to specifically bind an antigen on its own, whether the variable domain is present as a single immunoglobulin variable domain alone, or as a single immunoglobulin variable domain in association with one or more additional polypeptide sequences. A “human immunoglobulin variable domain” as the term is used herein does not encompass a “humanized” immunoglobulin polypeptide, i.e., a non-human (e.g., mouse, camel, etc.) immunoglobulin that has been modified in the constant regions to render it less immunogenic in humans.

As used herein, the phrase “sequence characteristic of immunoglobulin variable domains” refers to an amino acid sequence that is homologous, over 20 or more, 25 or more, 30 or more, 35 or more, 40 or more, 45 or more, or even 50 or more contiguous amino acids, to a sequence comprised by an immunoglobulin variable domain sequence.

As used herein, the term “bi-valent” means that an antigen-binding antibody polypeptide has two antigen-specific binding sites. The epitopes recognized by the antigen-binding sites can be the same or different. When the antibody polypeptide binds two different epitopes (present on different antigens, or, alternatively, on the same antigen) via the respective two antigen-specific binding sites, the antibody polypeptide is termed “dual-specific.”

As used herein the term “tetravalent” means that an antigen-binding polypeptide has four antigen-specific binding sites. The epitopes recognized by the antigen-binding sites can be the same or different. A “dual-specific” tetravalent antibody polypeptide has two binding sites for one epitope or antigen and two binding sites for a different epitope or antigen.

As used herein, a “tetravalent, dual-specific antigen-binding polypeptide construct” has a structure analogous to a naturally occurring IgG, in that it has two antigen-binding arms joined by heavy and light chain constant domains. However, unlike naturally-occurring IgG, each arm has two antigen-binding domains, one specific for a first antigen and one specific for a second antigen. In the tetravalent, dual-specific antigen-binding polypeptide constructs described herein, each of the antigen-binding domains is a single domain antibody, i.e., the antigen-binding domains do not pair together to form a single binding site, e.g., as in scFvs.

As used herein, the term “IgG format” refers to an artificial antigen-binding polypeptide with a structure analogous to a naturally-occurring IgG in that the construct has two antigen-binding arms joined by heavy and light chain constant domains that associate with each other. As described herein, an antigen-binding polypeptide in the IgG format is comprised of four polypeptide chains: two copies of a first fusion protein comprising a single-domain antibody polypeptide that binds a first antigen or epitope, fused to an IgG heavy chain constant domain (e.g., one comprising C_{H}1-C_{H}2-C_{H}3); and two copies of a second fusion protein comprising a single domain antibody polypeptide that binds a second antigen, fused to a light chain constant domain (e.g., C_{λ} or C_{κ}). In this format, when co-expressed in a cell, the heavy chain constant domains disulfide bond to each other, and each of these heavy chain constant domains also disulfide bonds to a light chain constant domain. Antigen-binding polypeptides in the IgG format are tetravalent as the term is used herein; the single domain antibodies fused to the constant domains can be selected to bind different antigens (e.g., dAb1, fused to heavy chain constant domain, binds one antigen, dAb2, fused to light chain constant domain binds another antigen), different epitopes on the same antigen (e.g., dAb1, fused to heavy chain constant domain, binds one epitope on an antigen, dAb2, fused to light chain constant domain binds another epitpoe on the same antigen), or, alternatively, all four can bind the same epitope on the same antigen (dAb1 and dAb2 bind the same epitope on the same antigen).

As used herein, the term “Fab format” refers to a bi-valent antibody polypeptide construct in which one single-domain antibody is fused to a light chain constant domain C_{L} (e.g., C_{λ} or C_{κ}), another single domain antibody is fused to a heavy chain C_{H}1 constant domain, and the respective C_{H}1 and C_{L} constant domains are disulfide bonded to each other. The single domain antibodies can be selected to bind different antigens (generating a dual-specific Fab format), different epitopes on the same antigen (also dual-specific) or the same epitope on the same antigen. An example of a Fab format dual-specific antibody polypeptide comprises, e.g., an anti-TNF-α single domain antibody described herein, fused, for example, to a C_{λ} light chain, and an anti-VEGF single domain antibody as described herein, fused to human heavy chain C_{H}1 constant domain, wherein the two fusion proteins are disulfide bonded to each other via their respective constant domains. In antibody polypeptide constructs of this format, the antigen-binding domains do not pair together to form a single binding site, e.g., as in scFvs; rather, each single domain antibody can bind antigen on its own, making the construct bi-valent.

By “rheumatoid arthritis” (RA) is meant a disease which involves inflammation in the lining of the joints and/or other internal organs. RA typically affects many different joints. It is typically chronic, and can be a disease of flare-ups. RA is a systemic disease that affects the entire body and is one of the most common forms of arthritis. It is characterized by the inflammation of the membrane lining the joint, which causes pain, stiffness, warmth, redness and swelling. The inflamed joint lining, the synovium, can invade and damage bone and cartilage. Inflammatory cells release enzymes that may digest bone and cartilage. The involved joint can lose its shape and alignment, resulting in pain and loss of movement. Symptoms include inflammation of joints, swelling, difficulty moving and pain. Other symptoms include loss of appetite, fever, loss of energy, anemia. Other features include lumps (rheumatoid nodules) under the skin in areas subject to pressure (e.g., back of elbows). Rheumatoid arthritis is clinically scored on the basis of several clinically accepted scales, such as those described in U.S. Pat. No. 5,698,195, which is incorporated herein by reference. Briefly, clinical response studies can assess the following parameters:

1. Number of tender joints and assessment of pain/tenderness

The following scoring is used:
0=No pain/tenderness
1=Mild pain. The patient says it is tender upon questioning.
2=Moderate pain. The patient says it is tender and winces.
3=Severe pain. The patient says it is tender and winces and withdraws.
2. Number of swollen joints

Both tenderness and swelling are evaluated for each joint separately.
3. Duration of morning stiffness (in minutes)
4. Grip strength
5. Visual analog pain scale (0-10 cm)
6. Patients and blinded evaluators are asked to assess the clinical response to the drug. Clinical response is assessed using a subjective scoring system as follows:

5=Excellent response (best possible anticipated response)

4=Good response (less than best possible anticipated response)

3=Fair response (definite improvement but could be better)

2=No response (no effect)

1=Worsening (disease worse)

The cause of rheumatoid arthritis is not yet known. However, it is known that RA is an autoimmune disease, resulting in the immune system attacking healthy joint tissue and causing inflammation and subsequent joint damage. Many people with RA have a certain genetic marker called HLA-DR4.

As used herein, the phrase “TNF-α related disorder” refers to a disease or disorder in which the administration of an agent that neutralizes or antagonizes the function of TNF-α is effective, alone or in conjunction with one or more additional agents or treatments, to treat such disorder as the term “treatment” is defined herein.

As used herein, the terms “treating” or “treatment” refer to a prevention of the onset of disease or a symptom of disease, inhibition of the progression of a disease or a symptom of a disease, or the reversal of disease or a disease symptom.

As used herein, the phrase “prevention of the onset of disease” means that one or more symptoms or measurable parameters of a given disease, e.g., rheumatoid arthritis, does not occur in an individual predisposed to such disease.

As used herein, the phrase “inhibition of the progression of disease” means that treatment with an agent either halts or slows the increase in severity of symptoms of a disease which has already manifested itself in the individual being treated, relative to progression in the absence of such treatment.

As used herein, the phrase “reversal of disease” means that one or more symptoms or measurable parameters of disease improves following administration of an agent, relative to that symptom or parameter prior to such administration. An “improvement” in a symptom or measurable parameter is evidenced by a statistically significant, but preferably at least a 10%, favorable difference in such a measurable parameter.

Measurable parameters can include, for example, both those that are directly measurable as well as those that are indirectly measurable. Non-limiting examples of directly measurable parameters include joint size, joint mobility, arthritic and histopathological scores or indicia and serum levels of an indicator, such as a cytokine. Indirectly measurable parameters include, for example, patient perception of discomfort or lack of mobility or a clinically accepted scale for rating disease severity.

As used herein, an “increase” in a parameter, e.g., an arthritic score or other measurable parameter, refers to a statistically significant increase in that parameter. Alternatively, an “increase” refers to at least a 10% increase. Similarly, a “decrease” in such a parameter refers to a statistically significant decrease in the parameter, or alternatively, to at least a 10% reduction.

As used herein, the term “antagonizes” means that an agent interferes with an activity. Where the activity is that of, for example, TNF-α, VEGF or another biologically active molecule or cytokine, the term encompasses inhibition (by at least 10%) of an activity of that molecule or cytokine, including as non-limiting examples, binding to or interaction with a receptor (in vitro or on a cell surface in culture or in vivo), intracellular signaling, cytotoxicity, mitogenesis, or other downstream effect or process (e.g., gene activation) mediated by that molecule or cytokine. Antagonism encompasses interference with receptor binding by the factor, e.g., TNF, VEGF, etc., as well as interference with the activity of the factor when the factor is bound to a cell-surface receptor.

As used herein, the term “greater than or equal to” means that a value is either equal to another or is greater than that value in a statistically significant manner (p<0.1, preferably p<0.05, more preferably p<0.01). Where efficacy of a composition is compared to that of another composition in, for example, disease treatment or antagonism of receptor binding, the comparison should be made on an equimolar basis.

As used herein, “linked” refers to the attachment of a polymer moiety, such as PEG to an amino acid residue of an antibody polypeptide, e.g., a single domain antibody as described herein. Attachment of a PEG polymer to an amino acid residue of an antibody polypeptide, such as a single domain antibody, is referred to as “PEGylation” and may be achieved using several PEG attachment moieties including, but not limited to N-hydroxylsuccinimide (NHS) active ester, succinimidyl propionate (SPA), maleimide (MAL), vinyl sulfone (VS), or thiol. A PEG polymer, or other polymer, can be linked to an antibody polypeptide at either a predetermined position, or may be randomly linked to the antibody molecule. It is preferred, however, that the PEG polymer be linked to an antibody polypeptide at a predetermined position. A PEG polymer may be linked to any residue in an antibody polypeptide, however, it is preferable that the polymer is linked to either a lysine or cyseine, which is either naturally occurring in an antibody polypeptide, or which has been engineered into an antibody polypeptide, for example, by mutagenesis of a naturally occurring residue in an antibody polypeptide to either a cysteine or lysine. As used herein, “linked” can also refer to the association of two or more antibody single variable domain monomers to form a dimer, trimer, tetramer, or other multimer. dAb monomers can be linked to form a multimer by several methods known in the art including, but not limited to expression of the dAb monomers as a fusion protein, linkage of two or more monomers via a peptide linker between monomers, or by chemically joining monomers after translation either to each other directly or through a linker by disulfide bonds, or by linkage to a di-, tri- or multivalent linking moiety (e.g., a multi-arm PEG).

As used herein, the phrase “directly linked” with respect to a polymer “directly linked” to an antibody polypeptide, e.g., a single variable domain polypeptide, refers to a situation in which the polymer is attached to a residue which naturally part of the variable domain, e.g., not contained within a constant region, hinge region, or linker peptide. Conversely, as used herein, the phrase “indirectly linked” to an antibody polypeptide refers to a linkage of a polymer molecule to an antibody single variable domain wherein the polymer is not attached to an amino acid residue which is part of the naturally occurring variable region (e.g., can be attached to a hinge region). A polymer is “indirectly linked” if it is linked to the antibody polypeptide via a linking peptide, that is the polymer is not attached to an amino acid residue which is a part of the antibody itself. Alternatively a polymer is “indirectly linked” to an antibody polypeptide if it is linked to a C-terminal hinge region of the polypeptide, or attached to any residues of a constant region which may be present as part of the antibody polypeptide. As used herein, the terms “homology” or “similarity” refer to the degree with which two nucleotide or amino acid sequences structurally resemble each other. As used herein, sequence “similarity” is a measure of the degree to which amino acid sequences share similar amino acid residues at corresponding positions in an alignment of the sequences. Amino acids are similar to each other where their side chains are similar. Specifically, “similarity” encompasses amino acids that are conservative substitutes for each other. A “conservative” substitution is any substitution that has a positive score in the blosum62 substitution matrix (Hentikoff and Hentikoff, 1992, Proc. Natl. Acad. Sci. USA 89: 10915-10919). By the statement “sequence A is n % similar to sequence B” is meant that n % of the positions of an optimal global alignment between sequences A and B consists of identical amino acids or conservative substitutions. Optimal global alignments can be performed using the following parameters in the Needleman-Wunsch alignment algorithm:

For polypeptides:
Substitution matrix: blosum62.
Gap scoring function: -A -B*LG, where A=11 (the gap penalty), B=1 (the gap length penalty) and LG is the length of the gap.

For nucleotide sequences:
Substitution matrix: 10 for matches, 0 for mismatches.
Gap scoring function: -A -B*LG where A=50 (the gap penalty), B=3 (the gap length penalty) and LG is the length of the gap.

Typical conservative substitutions are among Met, Val, Leu and Ile; among Ser and Thr; among the residues Asp, Glu and Asn; among the residues Gln, Lys and Arg; or aromatic residues Phe and Tyr.

As used herein, two sequences are “homologous” or “similar” to each other where they have at least 85% sequence similarity to each other when aligned using either the Needleman-Wunsch algorithm or the “BLAST 2 sequences” algorithm described by Tatusova & Madden, 1999, FEMS Microbiol Lett. 174:247-250. Where amino acid sequences are aligned using the “BLAST 2 sequences algorithm,” the Blosum 62 matrix is the default matrix.

As used herein, the terms “low stringency,” “medium stringency,” “high stringency,” or “very high stringency conditions” describe conditions for nucleic acid hybridization and washing. Guidance for performing hybridization reactions can be found in Current Protocols in Molecular Biology, John Wiley & Sons, N.Y. (1989), 6.3.1-6.3.6, which is incorporated herein by reference in its entirety. Aqueous and nonaqueous methods are described in that reference and either can be used. Specific hybridization conditions referred to herein are as follows: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and preferably (4) very high stringency hybridization conditions are 0.5M sodium phosphate, 7% SDS at 65° C., followed by one or more washes at 0.2X SSC, 1% SDS at 65° C.

As used herein, the phrase “at a concentration of means that a given polypeptide is dissolved in solution (preferably aqueous solution) at the recited mass or molar amount per unit volume. A polypeptide that is present “at a concentration of X” or “at a concentration of at least X” is therefore exclusive of both dried and crystallized preparations of a polypeptide.

As used herein, the term “repertoire” refers to a collection of diverse variants, for example polypeptide variants which differ in their primary sequence. A library used in the present invention will encompass a repertoire of polypeptides comprising at least 1000 members.

As used herein, the term “library” refers to a mixture of heterogeneous polypeptides or nucleic acids. The library is composed of members, each of which have a single polypeptide or nucleic acid sequence. To this extent, library is synonymous with repertoire. Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. Preferably, each individual organism or cell contains only one or a limited number of library members. Advantageously, the nucleic acids are incorporated into expression vectors, in order to allow expression of the polypeptides encoded by the nucleic acids. In a preferred aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

As used herein, “polymer” refers to a macromolecule made up of repeating monomeric units, and can refers to asynthetic or naturally occurring polymer such as an optionally substituted straight or branched chain polyalkylene, polyalkenylene, or polyoxyalkylene polymer or a branched or unbranched polysaccharide. A “polymer” as used herein, specifically refers to an optionally substituted or branched chain poly(ethylene glycol), poly(propylene glycol), or poly(vinyl alcohol) and derivatives thereof.

As used herein, “PEG” or “PEG polymer” refers to polyethylene glycol, and more specifically can refer to a derivitized form of PEG, including, but not limited to N-hydroxylsuccinimide (NHS) active esters of PEG such as succinimidyl propionate, benzotriazole active esters, PEG derivatized with maleimide, vinyl sulfones, or thiol groups. Particular PEG formulations can include PEG-O—CH₂CH₂CH₂—CO2—NHS; PEG-O—CH₂—NHS; PEG-O—CH₂CH₂—CO₂—NHS; PEG-S—CH₂CH₂—CO—NHS; PEG-O₂CNH—CH(R)—CO₂—NHS; PEG-NHCO—CH₂CH₂—CO—NHS; and PEG-O—CH₂—CO₂—NHS; where R is (CH₂)₄)NHCO₂(mPEG). PEG polymers useful in the invention may be linear molecules, or may be branched wherein multiple PEG moieties are present in a single polymer. Some particularly preferred PEG conformations that are useful in the invention include, but are not limited to the following:

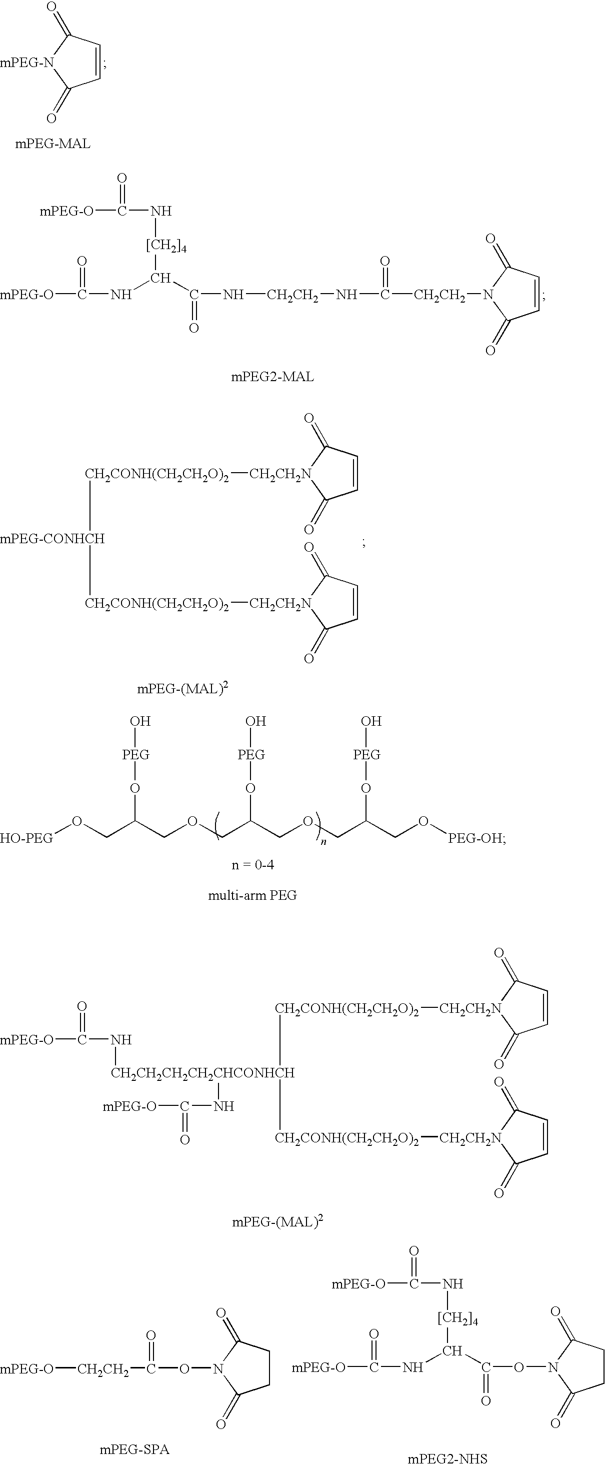

As used herein, a “sulfhydryl-selective reagent” is a reagent which is useful for the attachment of a PEG polymer to a thiol-containing amino acid. Thiol groups on the amino acid residue cysteine are particularly useful for interaction with a sulfhydryl-selective reagent. Sulfhydryl-selective reagents which are useful in the invention include, but are not limited to maleimide, vinyl sulfone, and thiol. The use of sulfhydryl-selective reagents for coupling to cysteine residues is known in the art and may be adapted as needed according to the present invention (See Eg., Zalipsky, 1995, Bioconjug. Chem. 6:150; Greenwald et al., 2000, Crit. Rev. Ther. Drug Carrier Syst. 17:101; Herman et al., 1994, Macromol. Chem. Phys. 195:203).

As used herein, the term “neutralizing,” when used in reference to a single immunoglobulin variable domain polypeptide as described herein, means that the polypeptide interferes with a measurable activity or function of the target antigen. A polypeptide is a “neutralizing” polypeptide if it reduces a measurable activity or function of the target antigen by at least 50%, and preferably at least 60%, 70%, 80%, 90%, 95% or more, up to and including 100% inhibition (i.e., no detectable effect or function of the target antigen). This reduction of a measurable activity or function of the target antigen can be assessed by one of skill in the art using standard methods of measuring one or more indicators of such activity or function. As an example, where the target is TNF-α, neutralizing activity can be assessed using a standard L929 cell killing assay or by measuring the ability of a single immunoglobulin variable domain to inhibit TNF-α-induced expression of ELAM-1 on HUVEC, which measures TNF-α-induced cellular activation. Analogous to “neutralizing” as used herein, “inhibit cell cytotoxicity” as used herein refers to a decrease in cell death as measured, for example, using a standard L929 cell killing assay, wherein cell cytotoxicity is inhibited were cell death is reduced by at least 10% or more.

As used herein, a “measurable activity or function of a target antigen” includes, but is not limited to, for example, cell signaling, enzymatic activity, binding activity, ligand-dependent internalization, cell killing, cell activation, promotion of cell survival, and gene expression. One of skill in the art can perform assays that measure such activities for a given target antigen. Preferably, “activity”, as used herein, is defined by (1) ND50 in a cell-based assay; (2) affinity for a target ligand, (3) ELISA binding, or (4) a receptor binding assay. Methods for performing these tests are known to those of skill in the art and are described in further detail herein below.

As used herein, “retains activity” refers to a level of activity of the PEG-linked antibody polypeptide, e.g., a single variable domain, which is at least 10% of the level of activity of a non-PEG-linked antibody polypeptide, preferably at least 20%, 30%, 40%, 50%, 60%, 70%, 80% and up to 90%, preferably up to 95%, 98%, and up to 100% of the activity of a non-PEG-linked antibody polypeptide of the same sequence, wherein activity is determined as described herein. More specifically, the activity of a PEG-linked antibody polypeptide compared to a non-PEG linked antibody polypeptide should be determined on an antibody molar basis; that is equivalent numbers of moles of each of the PEG-linked and non-PEG-linked antibody polypeptides should be used in each trial. In determining whether a particular PEG-linked antibody polypeptide “retains activity”, it is preferred that the activity of a PEG-linked antibody polypeptide be compared with the activity of the same antibody polypeptide in the absence of PEG.

As used herein, the terms “homodimer,” “homotrimer”, “homotetramer”, and “homomultimer” refer to molecules comprising two, three or more (e.g., four, five, etc.) monomers of a given single immunoglobulin variable domain polypeptide sequence, respectively. For example, a homodimer would include two copies of the same V_{H} sequence. A “monomer” of a single immunoglobulin variable domain polypeptide is a single V_{H} or V_{L} sequence that specifically binds antigen. The monomers in a homodimer, homotrimer, homotetramer, or homomultimer can be linked either by expression as a fusion polypeptide, e.g., with a peptide linker between monomers, or, by chemically joining monomers after translation either to each other directly or through a linker by disulfide bonds, or by linkage to a di-, tri- or multivalent linking moiety. In one embodiment, the monomers in a homodimer, trimer, tetramer, or multimer can be linked by a multi-arm PEG polymer, wherein each monomer of the dimer, trimer, tetramer, or multimer is linked as described above to a PEG moiety of the multi-arm PEG.

As used herein, the terms “heterodimer,” “heterotrimer”, “heterotetramer”, and “heteromultimer” refer to molecules comprising two, three or more (e.g., four, five, six, seven and up to eight or more) monomers of two or more different single immunoglobulin variable domain polypeptide sequence, respectively. For example, a heterodimer would include two V_{H} sequences, such as V_{H1} and V_{H2}, or may alternatively include a combination of V_{H} and V_{L}. Similar to a homodimer, trimer, or tetramer, the monomers in a heterodimer, heterotrimer, heterotetramer, or heteromultimer can be linked either by expression as a fusion polypeptide, e.g., with a peptide linker between monomers, or, by chemically joining monomers after translation either to each other directly or through a linker by disulfide bonds, or by linkage to a di-, tri- or multivalent linking moiety. In one embodiment, the monomers in a heterodimer, trimer, tetramer, or multimer can be linked by a multi-arm PEG polymer, wherein each monomer of the dimer, trimer, tetramer, or multimer is linked as described above to a PEG moiety of the multi-arm PEG.

“Half-life” The time taken for the serum concentration of the ligand to reduce by 50%, in vivo, for example due to degradation of the ligand and/or clearance or sequestration of the ligand by natural mechanisms. The ligands of the invention are stabilised in vivo and their half-life increased by binding to molecules which resist degradation and/or clearance or sequestration, such as serum albumin or PEG. Typically, however, such molecules are naturally occurring proteins which themselves have a long half-life in vivo. The half-life of a ligand is increased if its functional activity persists, in vivo, for a longer period than a similar ligand which is not specific for the half-life increasing molecule. Thus, a ligand specific for HSA and a target molecule is compared with the same ligand wherein the specificity for HSA is not present, that it does not bind HSA but binds another molecule. For example, it may bind a second epitope on the target molecule. Typically, the half life is increased by 10%, 20%, 30%, 40%, 50% or more. Increases in the range of 2×, 3×, 4×, 5×, 10×, 20×, 30×, 40×, 50× or more of the half life are possible. Alternatively, or in addition, increases in the range of up to 30×, 40×, 50×, 60×, 70×, 80×, 90×, 100×, 150× of the half life are possible. In the context of a PEG linked ligand, the PEG-linked ligand can have a half-life of between 0.25 and 170 hours, preferably between 1 and 100 hours, more preferably between 30 and 100 hours, and still more preferably between 50 and 100 hours, and up to 170, 180, 190, and 200 hours or more.

The phrase “substantially the same” when used to compare the T beta half life of a ligand with the T beta half life of serum albumin in a host means that the T beta half life of the ligand in a host varies no more than 50% from the T beta half life of serum albumin itself in the same host, preferably a human host, e.g., the T beta half life of such a ligand is no more than 50% less or no more than 50% greater than the T beta half life of serum albumin in a specified host. Preferably, when referring to the phrase “substantially the same”, the T beta half life of the ligand in a host varies no more than 20% to 10% from the half life of serum albumin itself, and more preferably, varies no more than 9%, 8%, 7%, 6%, 5%, 4%, 3%, 2%, or 1%, or less from the half life of serum albumin itself, or does not vary at all from the half life of serum albumin itself.

Alternatively, the phrase “not substantially the same” when used to compare the T beta half life of a ligand with the T beta half life of serum albumin in a host means that the T beta half life of the ligand in a host varies at least 50% from the T beta half life of serum albumin itself in the same host, preferably a human host, e.g., the T beta half life of the ligand is at least 50% less or at least 50% greater than the T beta half life of serum albumin in a specified host.

As used herein, “resistant to degradation” or “resists degradation” when used with respect to a PEG or other polymer linked dAb monomer or multimer means that the PEG- or other polymer-linked dAb monomer or multimer is degraded by no more than 10% when exposed to pepsin at pH 2.0 for 30 minutes and preferably not degraded at all. With specific reference to a PEG- or other polymer-linked dAb multimer (e.g., hetero- or homodimer, trimer, tetramer, etc) a molecule that is resistant to degradation is degraded by less than 5%, and is preferably not degraded at all in the presence of pepsin at pH 2.0 for 30 minutes.

As used herein, “hydrodynamic size” refers to the apparent size of a molecule (e.g., a protein molecule) based on the diffusion of the molecule through an aqueous solution. The diffusion, or motion of a protein through solution can be processed to derive an apparent size of the protein, where the size is given by the “Stokes radius” or “hydrodynamic radius” of the protein particle. The “hydrodynamic size” of a protein depends on both mass and shape (conformation), such that two proteins having the same molecular mass may have differing hydrodynamic sizes based on the overall conformation of the protein. The hydrodynamic size of a PEG-linked antibody polypeptide, e.g., a single variable domain (including antibody variable domain multimers as described herein), can be in the range of 24 kDa to 500 kDa; 30 to 500 kDa; 40 to 500 kDa; 50 to 500 kDa; 100 to 500 kDa; 150 to 500 kDa; 200 to 500 kDa; 250 to 500 kDa; 300 to 500 kDa; 350 to 500 kDa; 400 to 500 kDa and 450 to 500 kDa. Preferably the hydrodynamic size of a PEGylated dAb of the invention is 30 to 40 kDa; 70 to 80 kDa or 200 to 300 kDa. Where an antibody variable domain multimer is desired for use in imaging applications, the multimer should have a hydrodynamic size of between 50 and 100 kDa. Alternatively, where an antibody single domain multimer is desired for therapeutic applications, the multimer should have a hydrodynamic size of greater than 200 kDa.
Homogeneous immunoassay: An immunoassay in which analyte is detected without need for a step of separating bound and un-bound reagents.
TAR1-5-19 Dab: is a single domain antibody (Dab) specific for human TNF alpha.
TAR2h-10-27 Dab: is a single domain antibody (Dab) specific for human TNF receptor 1 (p55 receptor).
TAR1/TAR2 Fab, F(ab′)₂ or IgG are Fab, F(ab′)₂ or IgG formatted dual specific antibodies comprising TAR1-5-19 and TAR2h-10-27 Dabs as herein described.

### Dual-Specific Antibody Polypeptides:

The inventors have described, in their international patent application WO 2004/003019 a further improvement in dual specific ligands in which one specificity of the ligand is directed towards a protein or polypeptide present in vivo in an organism which can act to increase the half-life of the ligand by binding to it. WO 2004/003019 describes a dual-specific ligand comprising a first immunoglobulin single variable domain having a binding specificity to a first antigen or epitope and a second complementary immunoglobulin single variable domain having a binding activity to a second antigen or epitope, wherein one or both of said antigens or epitopes acts to increase the half-life of the ligand in vivo and wherein said first and second domains lack mutually complementary domains which share the same specificity, provided that said dual specific ligand does not consist of an anti-HSA VH domain and an anti-13 galactosidase Vκ domain.

Antigens or epitopes which increase the half-life of a ligand as described herein are advantageously present on proteins or polypeptides found in an organism in vivo.

Examples include extracellular matrix proteins, blood proteins, and proteins present in various tissues in the organism. The proteins act to reduce the rate of ligand clearance from the blood, for example by acting as bulking agents, or by anchoring the ligand to a desired site of action. Examples of antigens/epitopes which increase half-life in vivo are given in Annex 1 below.

Increased half-life is useful in in vivo applications of immunoglobulins, especially antibodies and most especially antibody fragments of small size. Such fragments (Fvs, disulphide bonded Fvs, Fabs, scFvs, dAbs) suffer from rapid clearance from the body; thus, whilst they are able to reach most parts of the body rapidly, and are quick to produce and easier to handle, their in vivo applications have been limited by their only brief persistence in vivo. The invention solves this problem by providing increased half-life of the ligands in vivo and consequently longer persistence times in the body of the functional activity of the ligand.

Methods for pharmacokinetic analysis and determination of ligand half-life will be familiar to those skilled in the art. Details may be found in Kenneth, A et al: Chemical Stability of Pharmaceuticals: A Handbook for Pharmacists and in Peters et al, Pharmacokinetc analysis: A Practical Approach (1996). Reference is also made to “Pharmacokinetics”, M Gibaldi & D Perron, published by Marcel Dekker, 2nd Rev. Edition (1982), which describes pharmacokinetic parameters such as t alpha and t beta half lives and area under the curve (AUC).

Half lives (T½ alpha and T½ beta) and AUC can be determined from a curve of serum concentration of ligand against time. The WinNonlin analysis package (available from Pharsight Corp., Mountain View, Calif., USA) can be used, for example, to model the curve. In a first phase (the alpha phase) the ligand is undergoing mainly distribution in the patient, with some elimination. A second phase (beta phase) is the terminal phase when the ligand has been distributed and the serum concentration is decreasing as the ligand is cleared from the patient. The t alpha half life is the half life of the first phase and the t beta half life is the half life of the second phase. Thus, advantageously, the present invention provides a ligand or a composition comprising a ligand according to the invention having a ta half-life in the range of 15 minutes or more. In one embodiment, the lower end of the range is 30 minutes, 45 minutes, 1 hour, 2 hours, 3 hours, 4 hours, 5 hours, 6 hours, 7 hours, 10 hours, 11 hours or 12 hours. In addition, or alternatively, a ligand or composition according to the invention will have a tα half life in the range of up to and including 12 hours. In one embodiment, the upper end of the range is 11, 10, 9, 8, 7, 6 or 5 hours. An example of a suitable range is 1 to 6 hours, 2 to 5 hours or 3 to 4 hours. Advantageously, the present invention provides a ligand or a composition comprising a ligand according to the invention having a tβ half-life in the range of 2.5 hours or more.

In one embodiment, the lower end of the range is 3 hours, 4 hours, 5 hours, 6 hours, 7 hours, 10 hours, 11 hours, or 12 hours. In addition, or alternatively, a ligand or composition according to the invention has a tβ half-life in the range of up to and including 21 days. In one embodiment, the upper end of the range is 12 hours, 24 hours, 2 days, 3 days, 5 days, 10 days, 15 days or 20 days. Advantageously a ligand or composition according to the invention will have a tβ half life in the range 12 to 60 hours.

In a further embodiment, it will be in the range 12 to 48 hours. a further embodiment still, it will be in the range 12 to 26 hours.

In addition, or alternatively to the above criteria, the present invention provides a ligand or a composition comprising a ligand according to the invention having an AUC value (area under the curve) in the range of 1 mg.min/ml or more. In one embodiment, the lower end of the range is 5, 10, 15, 20, 30, 100, 200 or 300 mg.min/ml. In addition, or alternatively, a ligand or composition according to the invention has an AUC in the range of up to 600 mg.min/ml.

n one embodiment, the upper end of the range is 500, 400, 300, 200, 150, 100, 75 or 50 mg.min/ml. Advantageously a ligand according to the invention will have a AUC in the range selected from the group consisting of the following: 15 to 150 mg.min/ml, 15 to 100 mg.min/ml, 15 to 75 mg. min/ml, and 15 to 50 mg.min/ml.

In a first embodiment, the dual specific ligand comprises two complementary variable domains, i.e. two variable domains that, in their natural environment, are capable of operating together as a cognate pair or group even if in the context of the present invention they bind separately to their cognate epitopes. For example, the complementary variable domains may be immunoglobulin heavy chain and light chain variable domains (VH and VL). VH and VL domains are advantageously provided by scFv or Fab antibody fragments. Variable domains may be linked together to form multivalent ligands by, for example: provision of a hinge region at the C-terminus of each V domain and disulphide bonding between cysteines in the hinge regions; or provision of dAbs each with a cysteine at the C-terminus of the domain, the cysteines being disulphide bonded together; or production of V-CH & V-CL to produce a Fab format; or use of peptide linkers (for example Gly4Ser linkers discussed hereinbelow) to produce dimers, trimers and further multimers. The inventors have found that the use of complementary variable domains allows the two domain surfaces to pack together and be sequestered from the solvent. Furthermore the complementary domains are able to stabilise each other. In addition, it allows the creation of dual- specific IgG antibodies without the disadvantages of hybrid hybridomas as used in the prior art, or the need to engineer heavy or light chains at the sub-unit interfaces.

The dual-specific ligands of the first aspect of the invention have at least one VH/VL pair. A bispecific IgG according to this invention will therefore comprise two such pairs, one pair on each arm of the Y-shaped molecule. Unlike conventional bispecific antibodies or diabodies, therefore, where the ratio of chains used is determinative in the success of the preparation thereof and leads to practical difficulties, the dual specific ligands of the invention are free from issues of chain balance. Chain imbalance in conventional bi-specific antibodies results from the association of two different VL chains with two different VH chains, where VL chain 1 together with VH chain 1 is able to bind to antigen or epitope 1 and VH chain 2 together with VH chain 2 is able to bind to antigen or epitope 2 and the two correct pairings are in some way linked to one another. Thus, only when VL chain 1 is paired with VH chain 1 and VL chain 2 is paired with VH chain 2 in a single molecule is bi- specificity created. Such bi-specific molecules can be created in two different ways. Firstly, they can be created by association of two existing VH/VL pairings that each bind to a different antigen or epitope (for example, in a bi-specific IgG). In this case the VH/VL pairings must come all together in a 1:1 ratio in order to create a population of molecules all of which are bi-specific. This never occurs (even when complementary CH domain is enhanced by “knobs into holes” engineering) leading to a mixture of bi-specific molecules and molecules that are only able to bind to one antigen or epitope but not the other. The second way of creating a bi-specific antibody is by the simultaneous association of two different VH chain with two different VL chains (for example in a bi-specific diabody). In this case, although there tends to be a preference for VL chain 1 to pair with VH chain 1 and VL chain 2 to pair with VH chain 2 (which can be enhanced by “knobs into holes” engineering of the VL and VH domains), this paring is never achieved in all molecules, leading to a mixed formulation whereby incorrect pairings occur that are unable to bind to either antigen or epitope.

Bi-specific antibodies constructed according to the dual-specific ligand approach according to the first aspect of the present invention overcome all of these problems because the binding to antigen or epitope 1 resides within the VH or VL domain and the binding to antigen or epitope 2 resides with the complementary VL or VH domain, respectively. Since VH and VL domains pair on a 1:1 basis all VH/VL pairings will be bi-specific and thus all formats constructed using these VH/VL pairings (Fv, scFvs, Fabs, minibodies, IgGs etc) will have 100% bi-specific activity.

In the context of the present invention, first and second “epitopes” are understood to be epitopes which are not the same and are not bound by a single monospecific ligand. In the first configuration of the invention, they are advantageously on different antigens, one of which acts to increase the half-life of the ligand in vivo. Likewise, the first and second antigens are advantageously not the same.

The dual specific ligands of the invention do not include ligands as described in WO 02/02773. Thus, the ligands of the present invention do not comprise complementary VH/VL pairs which bind any one or more antigens or epitopes co-operatively. Instead, the ligands according to the first aspect of the invention comprise a VH/VL complementary pair, wherein the V domains have different specificities.

Moreover, the ligands according to the first aspect of the invention comprise VH/VL complementary pairs having different specificities for non-structurally related epitopes or antigens. Structurally related epitopes or antigens are epitopes or antigens which possess sufficient structural similarity to be bound by a conventional VH/VL complementary pair which acts in a co-operative manner to bind an antigen or epitope, in the case of structurally related epitopes, the epitopes are sufficiently similar in structure that they “fit” into the same binding pocket formed at the antigen binding site of the VH/VL dimer.

In a second aspect, the present invention provides a ligand comprising a first immunoglobulin variable domain having a first antigen or epitope binding specificity and a second immunoglobulin variable domain having a second antigen or epitope binding specificity wherein one or both of said first and second variable domains bind to an antigen which increases the half-life of the ligand in vivo, and the variable domains are not complementary to one another.

In one embodiment, binding to one variable domain modulates the binding of the ligand to the second variable domain.

In this embodiment, the variable domains may be, for example, pairs of VH domains or pairs of VL domains. Binding of antigen at the first site may modulate, such as enhance or inhibit, binding of an antigen at the second site. For example, binding at the first site at least partially inhibits binding of an antigen at a second site. Such an embodiment, the ligand may for example be maintained in the body of a subject organism in vivo through binding to a protein which increases the half-life of the ligand until such a time as it becomes bound to the second target antigen and dissociates from the half-life increasing protein.

Modulation of binding in the above context is achieved as a consequence of the structural proximity of the antigen binding sites relative to one another. Such structural proximity can be achieved by the nature of the structural components linking the two or more antigen binding sites, eg by the provision of a ligand with a relatively rigid structure that holds the antigen binding sites in close proximity. Advantageously, the two or more antigen binding sites are in physically close proximity to one another such that one site modulates the binding of antigen at another site by a process which involves steric hindrance and/or conformational changes within the immunoglobulin molecule.

The first and the second antigen binding domains may be associated either covalently or non-covalently. In the case that the domains are covalently associated, then the association may be mediated for example by disulphide bonds or by a polypeptide linker such as (Gly4Ser)n, where n=from 1 to 8, eg, 2, 3, 4, 5 or 7.

Ligands according to this aspect of the invention may be combined into non-immunoglobulin multi ligand structures to form multivalent complexes, which bind target molecules with the same antigen, thereby providing superior avidity, while at least one variable domain binds an antigen to increase the half life of the multimer. For example natural bacterial receptors such as SpA have been used as scaffolds for the grafting of CDRs to generate ligands which bind specifically to one or more epitopes. Details of this procedure are described in U.S. Pat. No. 5,S31,012. Other suitable scaffolds include those based on fibronectin and affibodies. Details of suitable procedures are described in WO 98/58965. Other suitable scaffolds include lipocallin and CTLA4, as described in van den Beuken et al., J. Mol. Biol. (2001) 310, 591-601, and scaffolds such as those described in W00069907 (Medical Research Council), which are based for example on the ring structure of bacterial GroEL or other chaperone polypeptides.

Protein scaffolds may be combined, for example, CDRs may be grafted on to a CTLA4 scaffold and used together with immunoglobulin V_{H} or V_{L} domains to form a ligand.

Likewise, fibronectin, lipocallin and other scaffolds may be combined.

In the case that the variable domains are selected from V-gene repertoires selected for instance using phage display technology as herein described, then these variable domains can comprise a universal framework region, such that they may be recognised by a specific generic ligand as herein defined. The use of universal frameworks, generic ligands and the like is described in WO99/20749. In the present invention, reference to phage display includes the use of both phage and/or phagemids.

located within the structural loops of the variable domains. The polypeptide sequences of either variable domain may be altered by DNA shuffling or by mutation in order to enhance the interaction of each variable domain with its complementary pair.

In a preferred embodiment of the invention the ‘dual-specific ligand’ is a single chain Fv fragment. In an alternative embodiment of the invention, the ‘dual-specific ligand’ consists of a Fab region of an antibody. The term “Fab region” includes a Fab-like region where two VH or two VL domains are used.

The variable regions may be derived from antibodies directed against target antigens or epitopes. Alternatively they may be derived from a repertoire of single antibody domains such as those expressed on the surface of filamentous bacteriophage. Selection may be performed as described herein below and in the Examples.

Preparation of dAbs:

An aspect of the invention relates not only to dual-specific ligands in general, but also to various constructs of ligands that bind TNF-α alone, TNF-α and HSA or other half-life-extending polypeptide in the dual-specific format, and ligands that bind TNF-α and VEGF in the dual specific format. Ligands that bind VEGF and HSA or other half-life-extending polypeptide can also be prepared. The dual-specific TNF-α/NEGF construct can additionally comprise a binder for HSA or another half-life-extending molecule. In each of these embodiments, the individual ligands, i.e., those that bind TNF-α, HSA or VEGF, can be and are preferably, dAbs. The generation of such dAbs is discussed below and in the Examples.

In various aspects, the dAbs disclosed herein can be present in monomeric form, dimeric form, trimeric form, tetrameric form, or even in higher multimeric forms. In addition to the heterodimeric forms such as the dual specific constructs, multimeric constructs can be homomultimeric, i.e., homodimer, homotrimer, homotetramer, etc. Heterotrimers, heterotetramers and higher order heteromultimers are also specifically contemplated. Each of the various dAb conformations can additionally be complexed with additional moieties, such as polyethylene glycol (PEG) in order to further prolong the serum half-life of the polypeptide construct. PEGylation is known in the art and described herein.

Single immunoglobulin variable domains or dAbs are prepared in a number of ways. In a preferred aspect, the dAbs are human single immunoglobulin variable domains. For each of these approaches, well-known methods of preparing (e.g., amplifying, mutating, etc.) and manipulating nucleic acid sequences are applicable.

One means of preparing dAbs is to amplify and express the V_{H} or V_{L} region of a heavy chain or light chain gene for a cloned antibody known to bind the desired antigen. The boundaries of V_{H} and V_{L} domains are set out by Kabat et al. (1991, supra). The information regarding the boundaries of the V_{H} and V_{L} domains of heavy and light chain genes is used to design PCR primers that amplify the V domain from a cloned heavy or light chain coding sequence encoding an antibody known to bind a given antigen. The amplified V domain is inserted into a suitable expression vector, e.g., pHEN-1 (Hoogenboom et al., 1991, Nucleic Acids Res. 19: 4133-4137) and expressed, either alone or as a fusion with another polypeptide sequence. The expressed V_{H} or V_{L} domain is then screened for high affinity binding to the desired antigen in isolation from the remainder of the heavy or light chain polypeptide. For all aspects of the present invention, screening for binding is performed as known in the art or as described herein below.

A repertoire of V_{H} or V_{L} domains is screened by, for example, phage display, panning against the desired antigen. Methods for the construction of bacteriophage display libraries and lambda phage expression libraries are well known in the art, and taught, for example, by: McCafferty et al., 1990, Nature 348: 552; Kang et al., 1991, Proc. Natl. Acad. Sci. U.S.A., 88: 4363; Clackson et al., 1991, Nature 352: 624; Lowman et al., 1991, Biochemistry 30: 10832; Burton et al., 1991, Proc. Natl. Acad. Sci U.S.A. 88: 10134; Hoogenboom et al., 1991, Nucleic Acids Res. 19: 4133; Chang et al.,1991, J. Immunol. 147: 3610; Breitling et al., 1991, Gene 104: 147; Marks et al., 1991, J. Mol. Biol. 222: 581; Barbas et al., 1992, Proc. Natl. Acad. Sci. U.S.A. 89: 4457; Hawkins and Winter (1992) J. Immunol., 22: 867; Marks et al. (1992) J. Biol. Chem., 267: 16007; and Lerner et al. (1992) Science, 258: 1313. scFv phage libraries are taught, for example, by Huston et al., 1988, Proc. Natl. Acad. Sci U.S.A. 85: 5879-5883; Chaudhary et al., 1990, Proc. Natl. Acad. Sci U.S.A. 87: 1066-1070; McCafferty et al., 1990, supra; Clackson et al., 1991, supra; Marks et al., 1991, supra; Chiswell et al., 1992, Trends Biotech. 10: 80; and Marks et al., 1992, supra. Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys, supra).

The repertoire of V_{H} or V_{L} domains can be a naturally-occurring repertoire of immunoglobulin sequences or a synthetic repertoire. A naturally-occurring repertoire is one prepared, for example, from immunoglobulin-expressing cells harvested from one or more individuals. Such repertoires can be “naïve,” i.e., prepared, for example, from human fetal or newborn immunoglobulin-expressing cells, or rearranged, i.e., prepared from, for example, adult human B cells. Natural repertoires are described, for example, by Marks et al., 1991, J. Mol. Biol. 222: 581 and Vaughan et al., 1996, Nature Biotech. 14: 309. If desired, clones identified from a natural repertoire, or any repertoire, for that matter, that bind the target antigen are then subjected to mutagenesis and further screening in order to produce and select variants with improved binding characteristics.

Synthetic repertoires of single immunoglobulin variable domains are prepared by artificially introducing diversity into a cloned V domain. Synthetic repertoires are described, for example, by Hoogenboom & Winter, 1992, J. Mol. Biol. 227: 381; Barbas et al., 1992, Proc. Natl. Acad. Sci. U.S.A. 89: 4457; Nissim et al., 1994, EMBO J. 13: 692; Griffiths et al., 1994, EMBO J. 13: 3245; DeKriuf et al., 1995, J. Mol. Biol. 248: 97; and WO 99/20749.

The antigen binding domain of a conventional antibody comprises two separate regions: a heavy chain variable domain (V_{H}) and a light chain variable domain (V_{L}: which can be either V_{κ} or V_{λ}). The antigen binding site of such an antibody is formed by six polypeptide loops: three from the V_{H} domain (H1, H2 and H3) and three from the V_{L} domain (L1, L2 and L3). The boundaries of these loops are described, for example, in Kabat et al. (1991, supra). A diverse primary repertoire of V genes that encode the V_{H} and V_{L} domains is produced in vivo by the combinatorial rearrangement of gene segments. The V_{H} gene is produced by the recombination of three gene segments, V_{H}, D and J_{H}. In humans, there are approximately 51 functional V_{H} segments (Cook and Tomlinson (1995) Immunol Today 16: 237), 25 functional D segments (Corbett et al. (1997) J. Mol. Biol. 268: 69) and 6 functional J_{H} segments (Ravetch et al. (1981) Cell 27: 583), depending on the haplotype. The V_{H} segment encodes the region of the polypeptide chain which forms the first and second antigen binding loops of the V_{H} domain (H1 and H2), while the V_{H}, D and J_{H} segments combine to form the third antigen binding loop of the V_{H} domain (H3).

The V_{L} gene is produced by the recombination of only two gene segments, V_{L} and J_{L}. In humans, there are approximately 40 functional V_{κ} segments (Schäble and Zachau (1993) Biol. Chem. Hoppe-Seyler 374: 1001), 31 functional V_{λ} segments (Williams et al. (1996) J. Mol. Biol. 264: 220; Kawasaki et al. (1997) Genome Res. 7: 250), 5 functional J_{κ} segments (Hieter et al. (1982) J. Biol. Chem. 257: 1516) and 4 functional J_{λ} segments (Vasicek and Leder (1990) J. Exp. Med. 172: 609), depending on the haplotype. The V_{L} segment encodes the region of the polypeptide chain which forms the first and second antigen binding loops of the V_{L} domain (L1 and L2), while the V_{L} and J_{L} segments combine to form the third antigen binding loop of the V_{L} domain (L3). Antibodies selected from this primary repertoire are believed to be sufficiently diverse to bind almost all antigens with at least moderate affinity. High affinity antibodies are produced in vivo by “affinity maturation” of the rearranged genes, in which point mutations are generated and selected by the immune system on the basis of improved binding.

Analysis of the structures and sequences of antibodies has shown that five of the six antigen binding loops (H1, H2, L1, L2, L3) possess a limited number of main-chain conformations or canonical structures (Chothia and Lesk (1987) J. Mol. Biol. 196: 901; Chothia et al. (1989) Nature 342: 877). The main-chain conformations are determined by (i) the length of the antigen binding loop, and (ii) particular residues, or types of residue, at certain key position in the antigen binding loop and the antibody framework. Analysis of the loop lengths and key residues has enabled us to the predict the main-chain conformations of H1, H2, L1, L2 and L3 encoded by the majority of human antibody sequences (Chothia et al. (1992) J. Mol. Biol. 227: 799; Tomlinson et al. (1995) EMBO J. 14: 4628; Williams et al. (1996) J. Mol. Biol. 264: 220). Although the H3 region is much more diverse in terms of sequence, length and structure (due to the use of D segments), it also forms a limited number of main-chain conformations for short loop lengths which depend on the length and the presence of particular residues, or types of residue, at key positions in the loop and the antibody framework (Martin et al. (1996) J. Mol. Biol. 263: 800; Shirai et al. (1996) FEBS Letters 399: 1.

While, according to one embodiment of the invention, diversity can be added to synthetic repertoires at any site in the CDRs of the various antigen-binding loops, this approach results in a greater proportion of V domains that do not properly fold and therefore contribute to a lower proportion of molecules with the potential to bind antigen. An understanding of the residues contributing to the main chain conformation of the antigen-binding loops permits the identification of specific residues to diversify in a synthetic repertoire of V_{H} or V_{L} domains. That is, diversity is best introduced in residues that are not essential to maintaining the main chain conformation. As an example, for the diversification of loop L2, the conventional approach would be to diversify all the residues in the corresponding CDR (CDR2) as defined by Kabat et al. (1991, supra), some seven residues. However, for L2, it is known that positions 50 and 53 are diverse in naturally occurring antibodies and are observed to make contact with the antigen. The preferred approach would be to diversify only those two residues in this loop. This represents a significant improvement in terms of the functional diversity required to create a range of antigen binding specificities.

In one aspect, synthetic variable domain repertoires are prepared in V_{H} or V_{κ} backgrounds, based on artificially diversified germline V_{H} or V_{κ} sequences. For example, the V_{H} domain repertoire is based on cloned germline V_{H} gene segments V3-23/DP47 (Tomlinson et al., 1992, J. Mol. Biol. 227: 7768) and JH4b (see FIGS. 1 and 2). The V_{κ} domain repertoire is based, for example, on germline V_{κ} gene segments O2/O12/DPK9 (Cox et al., 1994, Eur. J. Immunol. 24: 827) and J_{κ}1 (see FIG. 3). Diversity is introduced into these or other gene segments by, for example, PCR mutagenesis. Diversity can be randomly introduced, for example, by error prone PCR (Hawkins, et al., 1992, J. Mol. Biol. 226: 889) or chemical mutagenesis. As discussed above, however it is preferred that the introduction of diversity is targeted to particular residues. It is further preferred that the desired residues are targeted by introduction of the codon NNK using mutagenic primers (using the IUPAC nomenclature, where N=G, A, T or C, and K=G or T), which encodes all amino acids and the TAG stop codon. Other codons which achieve similar ends are also of use, including the NNN codon (which leads to the production of the additional stop codons TGA and TAA), DVT codon ((A/G/T) (A/G/C)T), DVC codon ((A/G/T)(A/G/C)C), and DVY codon ((A/G/T)(A/G/C)(C/T). The DVT codon encodes 22% serine and 11% tyrosine, asgpargine, glycine, alanine, aspartate, threonine and cysteine, which most closely mimics the distribution of amino acid residues for the antigen binding sites of natural human antibodies. Repertoires are made using PCR primers having the selected degenerate codon or codons at each site to be diversified. PCR mutagenesis is well known in the art; however, considerations for primer design and PCR mutagenesis useful in the methods of the invention are discussed below in the section titled “PCR Mutagenesis.”

In one aspect, diversity is introduced into the sequence of human germline V_{H} gene segments V3-23/DP47 (Tomlinson et al., 1992, J. Mol. Biol. 227: 7768) and JH4b using the NNK codon at sites H30, H31, H33, H35, H50, H52, H52a, H53, H55, H56, H58, H95, H97 and H98, corresponding to diversity in CDRs 1, 2 and 3, as shown in FIG. 1.

In another aspect, diversity is also introduced into the sequence of human germline V_{H} gene segments V3-23/DP47 and JH4b, for example, using the NNK codon at sites H30, H31, H33, H35, H50, H52, H52a, H53, H55, H56, H58, H95, H97, H98, H99, H100, H100a and H100b, corresponding to diversity in CDRs 1, 2 and 3, as shown in FIG. 2.

In another aspect, diversity is introduced into the sequence of human germline V_{κ} gene segments O2/O12/DPK9 and J_{κ}1, for example, using the NNK codon at sites L30, L31, L32, L34, L50, L53, L91, L92, L93, L94 and L96, corresponding to diversity in CDRs 1, 2 and 3, as shown in FIG. 3.

Diversified repertoires are cloned into phage display vectors as known in the art and as described, for example, in WO 99/20749. In general, the nucleic acid molecules and vector constructs required for the performance of the present invention are available in the art and are constructed and manipulated as set forth in standard laboratory manuals, such as Sambrook et al. (1989). Molecular Cloning: A Laboratory Manual, Cold Spring Harbor, USA.

The manipulation of nucleic acids in the present invention is typically carried out in recombinant vectors. As used herein, “vector” refers to a discrete element that is used to introduce heterologous DNA into cells for the expression and/or replication thereof. Methods by which to select or construct and, subsequently, use such vectors are well known to one of skill in the art. Numerous vectors are publicly available, including bacterial plasmids, bacteriophage, artificial chromosomes and episomal vectors. Such vectors may be used for simple cloning and mutagenesis; alternatively, as is typical of vectors in which repertoire (or pre-repertoire) members of the invention are carried, a gene expression vector is employed. A vector of use according to the invention is selected to accommodate a polypeptide coding sequence of a desired size, typically from 0.25 kilobase (kb) to 40 kb in length. A suitable host cell is transformed with the vector after in vitro cloning manipulations. Each vector contains various functional components, which generally include a cloning (or “polylinker”) site, an origin of replication and at least one selectable marker gene. If a given vector is an expression vector, it additionally possesses one or more of the following: enhancer element, promoter, transcription termination and signal sequences, each positioned in the vicinity of the cloning site, such that they are operatively linked to the gene encoding a polypeptide repertoire member according to the invention.

Both cloning and expression vectors generally contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. The origin of replication from the plasmid pBR322 is suitable for most Gram-negative bacteria, the 2 micron plasmid origin is suitable for yeast, and various viral origins (e.g. SV 40, adenovirus) are useful for cloning vectors in mammalian cells. Generally, the origin of replication is not needed for mammalian expression vectors unless these are used in mammalian cells able to replicate high levels of DNA, such as COS cells.

Advantageously, a cloning or expression vector also contains a selection gene also referred to as selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available in the growth media.

Because the replication of vectors according to the present invention is most conveniently performed in E. coli, an E. coli-selectable marker, for example, the β-lactamase gene that confers resistance to the antibiotic ampicillin, is of use. These can be obtained from E. coli plasmids, such as pBR322 or a pUC plasmid such as pUC18 or pUC19.

Expression vectors usually contain a promoter that is recognized by the host organism and is operably linked to the coding sequence of interest. Such a promoter may be inducible or constitutive. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the β-lactamase and lactose promoter systems, alkaline phosphatase, the tryptophan (trp) promoter system and hybrid promoters such as the tac promoter. Promoters for use in bacterial systems will also generally contain a Shine-Dalgarno sequence operably linked to the coding sequence.

In libraries or repertoires as described herein, the preferred vectors are expression vectors that enable the expression of a nucleotide sequence corresponding to a polypeptide library member. Thus, selection is performed by separate propagation and expression of a single clone expressing the polypeptide library member or by use of any selection display system. As described above, a preferred selection display system uses bacteriophage display. Thus, phage or phagemid vectors can be used. Preferred vectors are phagemid vectors, which have an E. coli origin of replication (for double stranded replication) and also a phage origin of replication (for production of single-stranded DNA). The manipulation and expression of such vectors is well known in the art (Hoogenboom and Winter (1992) supra; Nissim et al. (1994) supra). Briefly, the vector contains a 13-lactamase or other selectable marker gene to confer selectivity on the phagemid, and a lac promoter upstream of a expression cassette that consists (N to C terminal) of a pelB leader sequence (which directs the expressed polypeptide to the periplasmic space), a multiple cloning site (for cloning the nucleotide version of the library member), optionally, one or more peptide tags (for detection), optionally, one or more TAG stop codons and the phage protein pIII. Using various suppressor and non-suppressor strains of E. coli and with the addition of glucose, iso-propyl thio-β-D-galactoside (IPTG) or a helper phage, such as VCS M13, the vector is able to replicate as a plasmid with no expression, produce large quantities of the polypeptide library member only, or produce phage, some of which contain at least one copy of the polypeptide-pIII fusion on their surface.

An example of a preferred vector is the pHEN1 phagemid vector (Hoogenboom et al., 1991, Nucl. Acids Res. 19: 4133-4137; sequence is available, e.g., as SEQ ID NO: 7 in WO 03/031611), in which the production of pill fusion protein is under the control of the LacZ promoter, which is inhibited in the presence of glucose and induced with IPTG. When grown in suppressor strains of E. coli, e.g., TG1, the gene III fusion protein is produced and packaged into phage, while growth in non-suppressor strains, e.g., HB2151, permits the secretion of soluble fusion protein into the bacterial periplasm and into the culture medium. Because the expression of gene III prevents later infection with helper phage, the bacteria harboring the phagemid vectors are propagated in the presence of glucose before infection with VCSM13 helper phage for phage rescue.

Construction of vectors according to the invention employs conventional ligation techniques. Isolated vectors or DNA fragments are cleaved, tailored, and re-ligated in the form desired to generate the required vector. If desired, sequence analysis to confirm that the correct sequences are present in the constructed vector is performed using standard methods. Suitable methods for constructing expression vectors, preparing in vitro transcripts, introducing DNA into host cells, and performing analyses for assessing expression and function are known to those skilled in the art. The presence of a gene sequence in a sample is detected, or its amplification and/or expression quantified by conventional methods, such as Southern or Northern analysis, Western blotting, dot blotting of DNA, RNA or protein, in situ hybridization, immunocytochemistry or sequence analysis of nucleic acid or protein molecules. Those skilled in the art will readily envisage how these methods may be modified, if desired.

PCR Mutagenesis:

The primer is complementary to a portion of a target molecule present in a pool of nucleic acid molecules used in the preparation of sets of nucleic acid repertoire members encoding polypeptide repertoire members. Most often, primers are prepared by synthetic methods, either chemical or enzymatic. Mutagenic oligonucleotide primers are generally 15 to 100 nucleotides in length, ideally from 20 to 40 nucleotides, although oligonucleotides of different length are of use.

Typically, selective hybridization occurs when two nucleic acid sequences are substantially complementary (at least about 65% complementary over a stretch of at least 14 to 25 nucleotides, preferably at least about 75%, more preferably at least about 85% or 90% complementary). See Kanehisa, 1984, Nucleic Acids Res. 12: 203, incorporated herein by reference. As a result, it is expected that a certain degree of mismatch at the priming site is tolerated. Such mismatch may be small, such as a mono-, di- or tri-nucleotide. Alternatively, it may comprise nucleotide loops, which are defined herein as regions in which mismatch encompasses an uninterrupted series of four or more nucleotides.

Overall, five factors influence the efficiency and selectivity of hybridization of the primer to a second nucleic acid molecule. These factors, which are (i) primer length, (ii) the nucleotide sequence and/or composition, (iii) hybridization temperature, (iv) buffer chemistry and (v) the potential for steric hindrance in the region to which the primer is required to hybridize, are important considerations when non-random priming sequences are designed.

There is a positive correlation between primer length and both the efficiency and accuracy with which a primer will anneal to a target sequence; longer sequences have a higher melting temperature (T_{M}) than do shorter ones, and are less likely to be repeated within a given target sequence, thereby minimizing promiscuous hybridization. Primer sequences with a high G-C content or that comprise palindromic sequences tend to self-hybridize, as do their intended target sites, since unimolecular, rather than bimolecular, hybridization kinetics are generally favored in solution; at the same time, it is important to design a primer containing sufficient numbers of G-C nucleotide pairings to bind the target sequence tightly, since each such pair is bound by three hydrogen bonds, rather than the two that are found when A and T bases pair. Hybridization temperature varies inversely with primer annealing efficiency, as does the concentration of organic solvents, e.g. formamide, that might be included in a hybridization mixture, while increases in salt concentration facilitate binding. Under stringent hybridization conditions, longer probes hybridize more efficiently than do shorter ones, which are sufficient under more permissive conditions. Stringent hybridization conditions for primers typically include salt concentrations of less than about 1M, more usually less than about 500 mM and preferably less than about 200 mM. Hybridization temperatures range from as low as 0° C. to greater than 22° C., greater than about 30° C., and (most often) in excess of about 37° C. Longer fragments may require higher hybridization temperatures for specific hybridization. As several factors affect the stringency of hybridization, the combination of parameters is more important than the absolute measure of any one alone.

Primers are designed with these considerations in mind While estimates of the relative merits of numerous sequences may be made mentally by one of skill in the art, computer programs have been designed to assist in the evaluation of these several parameters and the optimization of primer sequences. Examples of such programs are “PrimerSelect” of the DNAStar™ software package (DNAStar, Inc.; Madison, Wis.) and OLIGO 4.0 (National Biosciences, Inc.). Once designed, suitable oligonucleotides are prepared by a suitable method, e.g. the phosphoramidite method described by Beaucage and Carruthers, 1981, Tetrahedron Lett. 22: 1859) or the triester method according to Matteucci and Caruthers, 1981, J. Am. Chem. Soc. 103: 3185, both incorporated herein by reference, or by other chemical methods using either a commercial automated oligonucleotide synthesizer or, for example, VLSIPS™ technology.

PCR is performed using template DNA (at least lfg; more usefully, 1-1000 ng) and at least 25 pmol of oligonucleotide primers; it may be advantageous to use a larger amount of primer when the primer pool is heavily heterogeneous, as each sequence is represented by only a small fraction of the molecules of the pool, and amounts become limiting in the later amplification cycles. A typical reaction mixture includes: 2 μl of DNA, 25 pmol of oligonucleotide primer, 2.5 μl of 10×PCR buffer 1 (Perkin-Elmer), 0.4 μl of 1.25 μM dNTP, 0.15 μl (or 2.5 units) of Taq DNA polymerase (Perkin Elmer) and deionized water to a total volume of 25 μl. Mineral oil is overlaid and the PCR is performed using a programmable thermal cycler.

The length and temperature of each step of a PCR cycle, as well as the number of cycles, is adjusted in accordance to the stringency requirements in effect. Annealing temperature and timing are determined both by the efficiency with which a primer is expected to anneal to a template and the degree of mismatch that is to be tolerated; obviously, when nucleic acid molecules are simultaneously amplified and mutagenized, mismatch is required, at least in the first round of synthesis. In attempting to amplify a population of molecules using a mixed pool of mutagenic primers, the loss, under stringent (high-temperature) annealing conditions, of potential mutant products that would only result from low melting temperatures is weighed against the promiscuous annealing of primers to sequences other than the target site. The ability to optimize the stringency of primer annealing conditions is well within the knowledge of one of skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1-5 minutes, depending on the length of the amplified product). Final extension is generally for 4 minutes at 72° C., and may be followed by an indefinite (0-24 hour) step at 4° C.

Screening dAbs for Antigen Binding:

Following expression of a repertoire of dAbs on the surface of phage, selection is performed by contacting the phage repertoire with immobilized target antigen, washing to remove unbound phage, and propagation of the bound phage, the whole process frequently referred to as “panning.” Alternatively, phage are pre-selected for the expression of properly folded member variants by panning against an immobilized generic ligand (e.g., protein A or protein L) that is only bound by folded members. This has the advantage of reducing the proportion of non-functional members, thereby increasing the proportion of members likely to bind a target antigen. Pre-selection with generic ligands is taught in WO 99/20749. The screening of phage antibody libraries is generally described, for example, by Harrison et al., 1996, Meth. Enzymol. 267: 83-109.

Screening is commonly performed using purified antigen immobilized on a solid support, for example, plastic tubes or wells, or on a chromatography matrix, for example Sepharose™ (Pharmacia). Screening or selection can also be performed on complex antigens, such as the surface of cells (Marks et al., 1993, BioTechnology 11: 1145; de Kruif et al., 1995, Proc. Natl. Acad. Sci. U.S.A. 92: 3938). Another alternative involves selection by binding biotinylated antigen in solution, followed by capture on streptavidin-coated beads.

In a preferred aspect, panning is performed by immobilizing antigen (generic or specific) on tubes or wells in a plate, e.g., Nunc MAXISORP™ immunotube 8 well strips. Wells are coated with 150 μl of antigen (100 μg/ml in PBS) and incubated overnight. The wells are then washed 3 times with PBS and blocked with 400 μl PBS-2% skim milk (2% MPBS) at 37° C. for 2 hr. The wells are rinsed 3 times with PBS and phage are added in 2% MPBS. The mixture is incubated at room temperature for 90 minutes and the liquid, containing unbound phage, is removed. Wells are rinsed 10 times with PBS-0.1% tween 20, and then 10 times with PBS to remove detergent. Bound phage are eluted by adding 200 μl of freshly prepared 100 mM triethylamine, mixing well and incubating for 10 min at room temperature. Eluted phage are transferred to a tube containing 100 μl of 1M Tris-HCl, pH 7.4 and vortexed to neutralize the triethylamine. Exponentially-growing E. coli host cells (e.g., TG1) are infected with, for example, 150 ml of the eluted phage by incubating for 30 min at 37° C. Infected cells are spun down, resuspended in fresh medium and plated in top agarose. Phage plaques are eluted or picked into fresh cultures of host cells to propagate for analysis or for further rounds of selection. One or more rounds of plaque purification are performed if necessary to ensure pure populations of selected phage. Other screening approaches are described by Harrison et al., 1996, supra.

Following identification of phage expressing a single immunoglobulin variable domain that binds a desired target, if a phagemid vector such as pHEN1 has been used, the variable domain fusion protein are easily produced in soluble form by infecting non-suppressor strains of bacteria, e.g., HB2151 that permit the secretion of soluble gene III fusion protein. Alternatively, the V domain sequence can be sub-cloned into an appropriate expression vector to produce soluble protein according to methods known in the art.

Purification and Concentration of dAbs:

dAb polypeptides secreted into the periplasmic space or into the medium of bacteria are harvested and purified according to known methods (Harrison et al., 1996, supra). Skerra & Pluckthun (1988, Science 240: 1038) and Breitling et al. (1991, Gene 104: 147) describe the harvest of antibody polypeptides from the periplasm, and Better et al. (1988, Science 240: 1041) describes harvest from the culture supernatant. Purification can also be achieved by binding to generic ligands, such as protein A or Protein L. Alternatively, the variable domains can be expressed with a peptide tag, e.g., the Myc, HA or 6×-His tags, which facilitates purification by affinity chromatography.

Polypeptides are concentrated by several methods well known in the art, including, for example, ultrafiltration, diafiltration and tangential flow filtration. The process of ultrafiltration uses semi-permeable membranes and pressure to separate molecular species on the basis of size and shape. The pressure is provided by gas pressure or by centrifugation. Commercial ultrafiltration products are widely available, e.g., from Millipore (Bedford, Mass.; examples include the Centricon™ and Microcon™ concentrators) and Vivascience (Hannover, Germany; examples include the Vivaspin™ concentrators). By selection of a molecular weight cutoff smaller than the target polypeptide (usually ⅓ to ⅙ the molecular weight of the target polypeptide, although differences of as little as 10 kD can be used successfully), the polypeptide is retained when solvent and smaller solutes pass through the membrane. Thus, a molecular weight cutoff of about 5 kD is useful for concentration of dAb polypeptides described herein.

Diafiltration, which uses ultrafiltration membranes with a “washing” process, is used where it is desired to remove or exchange the salt or buffer in a polypeptide preparation. The polypeptide is concentrated by the passage of solvent and small solutes through the membrane, and remaining salts or buffer are removed by dilution of the retained polypeptide with a new buffer or salt solution or water, as desired, accompanied by continued ultrafiltration. In continuous diafiltration, new buffer is added at the same rate that filtrate passes through the membrane. A diafiltration volume is the volume of polypeptide solution prior to the start of diafiltration—using continuous diafiltration, greater than 99.5% of a fully permeable solute can be removed by washing through six diafiltration volumes with the new buffer. Alternatively, the process can be performed in a discontinuous manner, wherein the sample is repeatedly diluted and then filtered back to its original volume to remove or exchange salt or buffer and ultimately concentrate the polypeptide. Equipment for diafiltration and detailed methodologies for its use are available, for example, from Pall Life Sciences (Ann Arbor, Mich.) and Sartorius AG/Vivascience (Hannover, Germany).

Tangential flow filtration (TFF), also known as “cross-flow filtration,” also uses ultrafiltration membrane. Fluid containing the target polypeptide is pumped tangentially along the surface of the membrane. The pressure causes a portion of the fluid to pass through the membrane while the target polypeptide is retained above the filter. In contrast to standard ultrafiltration, however, the retained molecules do not accumulate on the surface of the membrane, but are carried along by the tangential flow. The solution that does not pass through the filter (containing the target polypeptide) can be repeatedly circulated across the membrane to achieve the desired degree of concentration. Equipment for TFF and detailed methodologies for its use are available, for example, from Millipore (e.g., the ProFlux M12™ Benchtop TFF system and the Pellicon™ systems), Pall Life Sciences (e.g., the Minim™ Tangential Flow Filtration system).

Protein concentration is measured in a number of ways that are well known in the art. These include, for example, amino acid analysis, absorbance at 280 nm, the “Bradford” and “Lowry” methods, and SDS-PAGE. The most accurate method is total hydrolysis followed by amino acid analysis by HPLC, concentration is then determined through comparison with the known sequence of the dAb polypeptide. While this method is the most accurate, it is expensive and time-consuming. Protein determination by measurement of UV absorbance at 280 nm is faster and much less expensive, yet relatively accurate and is preferred as a compromise over amino acid analysis. Absorbance at 280 nm was used to determine protein concentrations reported in the Examples described herein.

“Bradford” and “Lowry” protein assays (Bradford, 1976, Anal. Biochem. 72: 248-254; Lowry et al.,1951, J. Biol. Chem. 193: 265-275) compare sample protein concentration to a standard curve most often based on bovine serum albumin (BSA). These methods are less accurate, tending to undersetimate the concentration of single immunoglobulin variable domains. Their accuracy could be improved, however, by using a V_{H} or V_{κ} single domain polypeptide as a standard.

An additional protein assay method is the bicinchoninic acid assay described in U.S. Pat. No. 4,839,295 (incorporated herein by reference) and marketed by Pierce Biotechnology (Rockford, Ill.) as the “BCA Protein Assay” (e.g., Pierce Catalog No. 23227).

The SDS-PAGE method uses gel electrophoresis and Coomassie Blue staining in comparison to known concentration standards, e.g., known amounts of a single immunoglobulin variable domain polypeptide. Quantitation can be done by eye or by densitometry.

In a third aspect, the invention provides a method for producing a ligand comprising a first immunoglobulin single variable domain having a first binding specificity and a second single immunoglobulin single variable domain having a second (different) binding specificity, one or both of the binding specificities being specific for an antigen which increases the half-life of the ligand in vivo, the method comprising the steps of: (a) selecting a first variable domain by its ability to bind to a first epitope, (b) selecting a second variable region by its ability to bind to a second epitope, (c) combining the variable domains; and (d) selecting the ligand by its ability to bind to said first epitope and to said second epitope.

The ligand can bind to the first and second epitopes either simultaneously or, where there is competition between the binding domains for epitope binding, the binding of one domain may preclude the binding of another domain to its cognate epitope. In one embodiment, therefore, step (d) above requires simultaneous binding to both first and second (and possibly further) epitopes; in another embodiment, the binding to the first and second epitoes is not simultaneous.

The epitopes are preferably on separate antigens.

Ligands advantageously comprise VH/VL combinations, or VH/VH or VL/VL combinations of immunoglobulin variable domains, as described above. The ligands may moreover comprise camelid VHH domains, provided that the VHH domain which is specific for an antigen which increases the half-life of the ligand in vivo does not bind Hen egg white lysozyme (HEL), porcine pancreatic alpha-amylase or NmC-A; hog, BSA-linked RR6 ado 5 dye or S. mutates HG982 cells, as described in Conrath et al., (2001) JBC 276:7346-7350 and WO99/23221, neither of which describe the use of a specificity for an antigen which increases half-life to increase the half life of the ligand in vivo.

In one embodiment, said first variable domain is selected for binding to said first epitope in absence of a complementary variable domain (i.e., it is selected as a dAb as described herein above). In a further embodiment, said first variable domain is selected for binding to said first epitope/antigen in the presence of a third variable domain in which said third variable domain is different from said second variable domain and is complementary to the first domain. Similarly, the second domain may be selected in the absence or presence of a complementary variable domain.

The antigens or epitopes targeted by the ligands of the invention, in addition to the half life enhancing protein, may be any antigen or epitope but advantageously is an antigen or epitope that is targeted with therapeutic benefit. The invention provides ligands, including open conformation, closed conformation and isolated dAb monomer ligands, specific for any such target, particularly those targets further identified herein. Such targets may be, or be part of, polypeptides, proteins or nucleic acids, which may be naturally occurring or synthetic. In this respect, the ligand of the invention may bind the epiotpe or antigen and act as an antagonist or agonist (eg, EPO receptor agonist). One skilled in the art will appreciate that the choice is large and varied.

They may be for instance human or animal proteins, cytokines, cytokine receptors, enzymes co-factors for enzymes or DNA binding proteins. Suitable cytokines and growth factors that can be targeted by mono- or dual-specific binding polypeptides as described herein include but are not limited to: ApoE, Apo-SAA, BDNF, BLyS, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-01, insulin, IFN-γ, IGF-I, IGF-II, IL-, IL-1p, 20 IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin a, Inhibin B IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MIG, MIP1α, MIP1β, MIP3α, MIP3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF12, SDF1β, SCF, SCGF, stem cell factor (SCF), TARC, TGF-α, TGF-β, TGF-β2, TGF-β3, tumour necrosis factor (TNF), TNF-α, TNF-β3, TNF receptor I, TNF receptor II, TNIL-1, TPO, VEGF, VEGF receptor 1, VEGF receptor 2, VEGF receptor 3, GCP-2, GRO/MGSA, GRO-β, GRO-8, HCC1, 1-309, HER 1, HER 2, HER 3, HER 4, TACE recognition site, TNF BP-I and TNF BP-II, CD4, human chemokine receptors CXCR4 or CCR5, non-structural protein type 3 (NS3) from the hepatitis C virus, TNF-alpha, IgE, IFN-gamma, MMP-12, CEA, H. pylori, TB, influenza, Hepatitis E, MMP-12, internalizing receptors that are over-expressed on certain cells, such as the epidermal growth factor receptor (EGFR), ErBb2 receptor on tumor cells, an internalising cellular receptor, LDL receptor, FGF2 receptor, ErbB2 receptor, transferrin receptor, PDGF receptor, VEGF receptor, PsmAr, an extracellular matrix protein, elastin, fibronectin, laminin, α1-antitrypsin, tissue factor protease inhibitor, PDK1, GSK I, Bad, caspase-9, Forkhead, an antigen of Helicobacter pylori, an antigen of Mycobacterium tuberculosis, and an antigen of influenza virus as well as any target disclosed in Annex 2 or Annex 3 hereto, whether in combination as set forth in the Annexes, in a different combination, or individually.

As noted, preferred ligands include TNF-α and VEGF, alone, together, and/or with anti-HSA binding activity.

Cytokine receptors include receptors for the foregoing cytokines. It will be appreciated that this list is by no means exhaustive.

In one embodiment of the invention, the variable domains are derived from a respective antibody directed against the antigen or epitope. In a preferred embodiment the variable domains are derived from a repertoire of single variable antibody domains.

In a further aspect, the present invention provides one or more nucleic acid molecules encoding at least a dual-specific ligand as herein defined.

The dual specific ligand may be encoded on a single nucleic acid molecule; alternatively, each domain may be encoded by a separate nucleic acid molecule. Where the ligand is encoded by a single nucleic acid molecule, the domains may be expressed as a fusion polypeptide, in the manner of a scFv molecule, or may be separately expressed and subsequently linked together, for example using chemical linking agents. Ligands expressed from separate nucleic acids will be linked together by appropriate means.

The nucleic acid may further encode a signal sequence for export of the polypeptides from a host cell upon expression and may be fused with a surface component of a filamentous bacteriophage particle (or other component of a selection display system) upon expression.

In a further aspect the present invention provides a vector comprising nucleic acid encoding a dual specific ligand according to the present invention.

In a yet further aspect, the present invention provides a host cell transfected with a vector encoding a dual specific ligand according to the present invention.

Expression from such a vector may be configured to produce, for example on the surface of a bacteriophage particle, variable domains for selection. This allows selection of displayed variable regions and thus selection of ‘dual-specific ligands’ using the method of the present invention.

The present invention further provides a kit comprising at least a dual- specific ligand according to the present invention.

Dual-Specific ligands according to the present invention preferably comprise combinations of heavy and light chain domains. For example, the dual specific ligand may comprise a VH domain and a VL domain, which may be linked together in the form of an scFv. In addition, the ligands may comprise one or more CH or CL domains. For example, the ligands may comprise a CH1 domain, CH2 or CH3 domain, and/or a C_{L} domain, Cμ, Cμ2, Cμ3 or Cμ4 domains, or any combination thereof. A hinge region domain may also be included. Such combinations of domains may, for example, mimic natural antibodies, such as IgG or IgM, or fragments thereof, such as Fv, scFv, Fab or F(ab′)2 molecules. Other structures, such as a single arm of an IgG molecule comprising VH, VL, CH1 and C_{L} domains, are envisaged.

In a preferred embodiment of the invention, the variable regions are selected from single domain V gene repertoires. Generally the repertoire of single antibody domains is displayed on the surface of filamentous bacteriophage. In a preferred embodiment each single antibody domain is selected by binding of a phage repertoire to antigen.

In a preferred embodiment of the invention each single variable domain may be selected for binding to its target antigen or epitope in the absence of a complementary variable region. In an alternative embodiment, the single variable domains may be selected for binding to its target antigen or epitope in the presence of a complementary variable region. Thus the first single variable domain may be selected in the presence of a third complementary variable domain, and the second variable domain may be selected in the presence of a fourth complementary variable domain. The complementary third or fourth variable domain may be the natural cognate variable domain having the same specificity as the single domain being tested, or a non-cognate complementary domain—such as a “dummy” variable domain.

Preferably, the dual specific ligand of the invention comprises only two variable domains although several such ligands may be incorporated together into the same protein, for example two such ligands can be incorporated into an IgG or a multimeric immunoglobulin, such as IgM. Alternatively, in another embodiment a plurality of dual specific ligands are combined to form a multimer. For example, two different dual specific ligands are combined to create a tetra-specific molecule.

It will be appreciated by one skilled in the art that the light and heavy variable regions of a dual-specific ligand produced according to the method of the present invention may be on the same polypeptide chain, or alternatively, on different polypeptide chains. In the case that the variable regions are on different polypeptide chains, then they may be linked via a linker, generally a flexible linker (such as a polypeptide chain), a chemical linking group, or any other method known in the art.

In a further aspect, the present invention provides a composition comprising a dual specific ligand, obtainable by a method of the present invention, and a pharmaceutically acceptable carrier, diluent or excipient.

Moreover, the present invention provides a method for the treatment and/or prevention of disease using a ‘dual-specific ligand’ or a composition according to the present invention. In a second configuration, the present invention provides multispecific ligands which comprise at least two non-complementary variable domains. For example, the ligands may comprise a pair of VH domains or a pair of VL domains. Advantageously, the domains are of non-camelid origin; preferably they are human domains or comprise human framework regions (FWs) and one or more heterologous CDRs. CDRs and framework regions are those regions of an immunoglobulin variable domain as deemed in the Kabat database of Sequences of Proteins of Immunological Interest.

Preferred human framework regions are those encoded by germline gene segments DP47 and DPK9. Advantageously, FW 1, FW2 and FW3 of a VH or VL domain have the sequence of FW1, FW2 or FW3 from DP47 or DPK9. The human frameworks may optionally contain mutations, for example up to about 5 amino acid changes or up to about 10 amino acid changes collectively in the human frameworks used in the ligands of the invention.

The variable domains in the multispecific ligands according to the second configuration of the invention may be arranged in an open or a closed conformation; that is, they may be arranged such that the variable domains can bind their cognate ligands independently and simultaneously, or such that only one of the variable domains may bind its cognate ligand at any one time.

The inventors have realised that under certain structural conditions, non-complementary variable domains (for example two light chain variable domains or two heavy chain variable domains) may be present in a ligand such that binding of a first epitope to a first variable domain inhibits the binding of a second epitope to a second variable domain, even though such non-complementary domains do not operate together as a cognate pair.

Advantageously, the ligand comprises two or more pairs of variable domains; that is, it comprises at least four variable domains. Advantageously, the four variable domains comprise frameworks of human origin.

In a preferred embodiment, the human frameworks are identical to those of human germline sequences.

The present inventors consider that such antibodies will be of particular use in ligand binding assays for therapeutic and other uses.

Thus, in a first aspect of the second configuration, the present invention provides a method for producing a multispecific ligand comprising the steps of: a) selecting a first epitope binding domain by its ability to bind to a first epitope, b) selecting a second epitope binding domain by its ability to bind to a second epitope, c) combining the epitope binding domains; and d) selecting the closed conformation multispecific ligand by its ability to bind to said first second epitope and said second epitope.

In a further aspect of the second configuration, the invention provides method for preparing a closed conformation multi-specific ligand comprising a first epitope binding domain having a first epitope binding specificity and a non-complementary second epitope binding domain having a second epitope binding specificity, wherein the first and second binding specificities compete for epitope binding such that the closed conformation multi-specific ligand may not bind both epitopes simultaneously, said method comprising the steps of: a) selecting a first epitope binding domain by its ability to bind to a first epitope, b) selecting a second epitope binding domain by its ability to bind to a second epitope, c) combining the epitope binding domains such that the domains are in a closed conformation; and d) selecting the closed conformation multispecific ligand by its ability to bind to said first second epitope and said second epitope, but not to both said first and second epitopes simultaneously.

Moreover, the invention provides a closed conformation multi-specific ligand comprising a first epitope binding domain having a first epitope binding specificity and a non-complementary second epitope binding domain having a second epitope binding specificity, wherein the first and second binding specificities compete for epitope binding such that the closed conformation multi-specific ligand may not bind both epitopes simultaneously.

An alternative embodiment of the above aspect of the of the second configuration of the invention optionally comprises a further step (b1) comprising selecting a third or further epitope binding domain. In this way the multi-specific ligand produced, whether of open or closed conformation, comprises more than two epitope binding specificities. In a preferred aspect of the second configuration of the invention, where the multi-specific ligand comprises more than two epitope binding domains, at least two of said domains are in a closed conformation and compete for binding; other domains may compete for binding or may be free to associate independently with their cognate epitope(s).

According to the present invention the term ‘multi-specific ligand’ refers to a ligand which possesses more than one epitope binding specificity as herein defined.

As herein defined the term ‘closed conformation’ (multi-specific ligand) means that the epitope binding domains of the ligand are attached to or associated with each other, optionally by means of a protein skeleton, such that epitope binding by one epitope binding domain competes with epitope binding by another epitope binding domain. That is, cognate epitopes may be bound by each epitope binding domain individually but not simultaneosuly. The closed conformation of the ligand can be achieved using methods herein described.

“Open conformation” means that the epitope binding domains of the ligand are attached to or associated with each other, optionally by means of a protein skeleton, such that epitope binding by one epitope binding domain does not compete with epitope binding by another epitope binding domain.

As referred to herein, the term ‘competes’ means that the binding of a first epitope to its cognate epitope binding domain is inhibited when a second epitope is bound to its cognate epitope binding domain. For example, binding may be inhibited sterically, for example by physical blocking of a binding domain or by alteration of the structure or environment of a binding domain such that its affinity or avidity for an epitope is reduced.

In a further embodiment of the second configuration of the invention, the epitopes may displace each other on binding. For example, a first epitope may be present on an antigen which, on binding to its cognate first binding domain, causes steric hindrance of a second binding domain, or a coformational change therein, which displaces the epitope bound to the second binding domain.

Advantageously, binding is reduced by 25% or more, advantageously 40%, 50%, 60%, 70%, 80%, 90% or more, and preferably up to 100% or nearly so, such that binding is completely inhibited. Binding of epitopes can be measured by conventional antigen binding assays, such as ELISA, by fluorescence based techniques, including FRET, or by techniques such as suface plasmon resonance which measure the mass of molecules.

According to the method of the present invention, advantageously, each epitope binding domain is of a different epitope binding specificity.

In the context of the present invention, first and second “epitopes” are understood to be epitopes which are not the same and are not bound by a single monospecific ligand. They may be on different antigens or on the same antigen, but separated by a sufficient distance that they do not form a single entity that could be bound by a single mono-specific VH/VL binding pair of a conventional antibody. Experimentally, if both of the individual variable domains in single chain antibody form (domain antibodies or dAbs) are separately competed by a monospecific VH/VL ligand against two epitopes then those two epitopes are not sufficiently far apart to be considered separate epitopes according to the present invention.

The closed conformation multispecific ligands of the invention do not include ligands as described in WO 02/02773. Thus, the ligands of the present invention do not comprise complementary VH/VL pairs which bind any one or more antigens or epitopes co-operatively. Instead, the ligands according to the invention preferably comprise non-complementary VH or VL pairs. Advantageously, each VH or VL domain in each VH or VL pair has a different epitope binding specificity, and the epitope binding sites are so arranged that the binding of an epitope at one site competes with the binding of an epitope at another site.

According to the present invention, advantageously, each epitope binding domain comprises an immunoglobulin variable domain. More advantageously, each immunoglobulin variable domain will be either a variable light chain domain (VL) or a variable heavy chain domain VH. In the second configuration of the present invention, the immunoglobulin domains when present on a ligand according to the present invention are non-complementary, that is they do not associate to form a VH/VL antigen binding site. Thus, multi-specific ligands as deemed in the second configuration of the invention comprise immunoglobulin domains of the same sub-type, that is either variable light chain domains (VL) or variable heavy chain domains (VH). Moreover, where the ligand according to the invention is in the closed conformation, the immunoglobulin domains may be of the camelid VHH type.

In an alternative embodiment, the ligand(s) according to the invention do not comprise a camelid VHH domain. More particularly, the ligand(s) of the invention do not comprise one or more amino acid residues that are specific to camelid VHH domains as compared to human VH domains.

Advantageously, the single variable domains are derived from antibodies selected for binding activity against different antigens or epitopes. For example, the variable domains may be isolated at least in part by human immunisation. Alternative methods are known in the art, including isolation from human antibody libraries and synthesis of artificial antibody genes.

The variable domains advantageously bind superantigens, such as protein A or protein L. Binding to superantigens is a property of correctly folded antibody variable domains, and allows such domains to be isolated from, for example, libraries of recombinant or mutant domains. Epitope binding domains according to the present invention comprise a protein scaffold and epitope interaction sites (which are advantageously on the surface of the protein scaffold). Epitope binding domains may also be based on protein scaffolds or skeletons other than immunoglobulin domains. For example natural bacterial receptors such as SpA have been used as scaffolds for the grafting of CDRs to generate ligands which bind specifically to one or more epitopes. Details of this procedure are described in U.S. Pat. No. 5,831,012. Other suitable scaffolds include those based on fibronectin and affibodies. Details of suitable procedures are described in WO 98/58965. Other suitable scaffolds include lipocallin and CTLA4, as described in van den Beuken et al., J. Mol. Biol. (2001) 310, 591-601, and scaffolds such as those described in W00069907 (Medical Research Council), which are based for example on the ring structure of bacterial GroEL or other chaperone polypeptides. Protein scaffolds may be combined; for example, CDRs may be grafted on to a CTLA4 scaffold and used together with immunoglobulin VH or VL domains to form a multivalent ligand. Likewise, fibronectin, lipocallin and other scaffolds may be combined.

It will be appreciated by one skilled in the art that the epitope binding domains of a closed conformation multispecific ligand produced according to the method of the present invention may be on the same polypeptide chain, or alternatively, on different polypeptide chains. In the case that the variable regions are on different polypeptide chains, then they may be linked via a linker, advantageously a flexible linker (such as a polypeptide chain), a chemical linking group, or any other method known in the art.

The first and the second epitope binding domains may be associated either covalently or non-covalently. In the case that the domains are covalently associated, then the association may be mediated for example by disulphide bonds.

In the second configuration of the invention, the first and the second epitopes are preferably different. They may be, or be part of, polypeptides, proteins or nucleic acids, which may be naturally occurring or synthetic. In this respect, the ligand of the invention may bind an epitope or antigen and act as an antagonist or agonist (eg, EPO receptor agonist). The epitope binding domains of the ligand in one embodiment have the same epitope specificity, and may for example simultaneously bind their epitope when multiple copies of the epitope are present on the same antigen. In another embodiment, these epitopes are provided on different antigens such that the ligand can bind the epitopes and bridge the antigens. One skilled in the art will appreciate that the choice of epitopes and antigens is large and varied. They may be for instance human or animal proteins, cytokines, cytokine receptors, enzymes co-factors for enzymes or DNA binding proteins.

Suitable cytokines and growth factors that can be targeted by mono- or dual-specific binding polypeptides as described herein include but are not limited to: ApoE, Apo-SAA, BDNF, BLyS, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-01, insulin, IFN-γ, IGF-I, IGF-II, IL-, IL-1p, 20 IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin B IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MIG, MIP1α, MIP1β, MIP3α, MIP3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF12, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF receptor 1, VEGF receptor 2, VEGF receptor 3, GCP-2, GRO/MGSA, GRO-β, HCC1, 1-309, HER 1, HER 2, HER 3, HER 4, TACE recognition site, TNF BP-I and TNF BP-II, CD4, human chemokine receptors CXCR4 or CCR5, non-structural protein type 3 (NS3) from the hepatitis C virus, TNF-alpha, IgE, IFN-gamma, MMP-12, CEA, H. pylori, TB, influenza, Hepatitis E, MMP-12, internalizing receptors that are over-expressed on certain cells, such as the epidermal growth factor receptor (EGFR), ErBb2 receptor on tumor cells, an internalising cellular receptor, LDL receptor, FGF2 receptor, ErbB2 receptor, transferrin receptor, PDGF receptor, VEGF receptor, PsmAr, an extracellular matrix protein, elastin, fibronectin, laminin, al-antitrypsin, tissue factor protease inhibitor, PDK1, GSK1, Bad, caspase-9, Forkhead, an antigen of Helicobacter pylori, an antigen of Mycobacterium tuberculosis, and an antigen of influenza virus as well as any target disclosed in Annex 2 OR Annex 3 hereto, whether in combination as set forth in the Annexes, in a different combination, or individually.

Cytokine receptors include receptors for the foregoing cytokines, e.g. IL-1 R1; IL-GR; IL-10R; IL-18R, as well as receptors for cytokines set forth in Annex 2 or Annex 3 and also receptors disclosed in Annex 2 and 3.

It will be appreciated that this list is by no means exhaustive. Where the multispecific ligand binds to two epitopes (on the same or different antigens), the antigen(s) may be selected from this list.

Advantageously, dual specific ligands may be used to target cytokines and other molecules which cooperate synergistically in therapeutic situations in the body of an organism. The invention therefore provides a method for synergising the activity of two or more cytokines, comprising administering a dual specific ligand capable of binding to said two or more cytokines. In this aspect of the invention, the dual specific ligand may be any dual specific ligand, including a ligand composed of complementary and/or non-complementary domains, a ligand in an open conformation, and a ligand in a closed conformation. For example, this aspect of the invention relates to combinations of VH domains and VL domains, VH domains only and VL domains only.

Synergy in a therapeutic context may be achieved in a number of ways. For example, target combinations may be therapeutically active only if both targets are targeted by the ligand, whereas targeting one target alone is not therapeutically effective. In another embodiment, one target alone may provide some low or minimal therapeutic effect, but together with a second target the combination provides a synergistic increase in therapeutic effect.

Preferably, the cytokines bound by the dual specific ligands of this aspect of the invention are selected from the list shown in Annex 2.

Moreover, dual specific ligands may be used in oncology applications, where one specificity targets CD89, which is expressed by cytotoxic cells, and the other is tumor specific. Examples of tumor antigens which may be targeted are given in Annex 3.

In one embodiment of the second configuration of the invention, the variable domains are derived from an antibody directed against the first and/or second antigen or epitope. In a preferred embodiment the variable domains are derived from a repertoire of single variable antibody domains. In one example, the repertoire is a repertoire that is not created in an animal or a synthetic repertoire. In another example, the single variable domains are not isolated (at least in part) by animal immunization. Thus, the single domains can be isolated from a nerve library.

The second configuration of the invention, in another aspect, provides a multi-specific ligand comprising a first epitope binding domain having a first epitope binding specificity and a non-complementary second epitope binding domain having a second epitope binding specificity. The first and second binding specificities may be the same or different.

In a further aspect, the present invention provides a closed conformation multi-specific ligand comprising a first epitope binding domain having a first epitope binding specificity and a non-complementary second epitope binding domain having a second epitope binding specificity wherein the first and second binding specificities are capable of competing for epitope binding such that the closed conformation multi-specific ligand cannot bind both epitopes simultaneously.

In a still further aspect, the invention provides open conformation ligands comprising non-complementary binding domains, wherein the domains are specific for a different epitope on the same target. Such ligands bind to targets with increased avidity.

Similarly, the invention provides multivalent ligands comprising non-complementary binding domains specific for the same epitope and directed to targets which comprise multiple copies of said epitope, such as IL-5, PDGF-AA, PDGF-BB, TGF β, TGF β2, TGF β3 and TNFα, for example human TNF Receptor I and human TNFα.

In a similar aspect, ligands according to the invention can be configured to bind individual epitopes with low affinity, such that binding to individual epitopes is not therapeutically significant; but the increased avidity resulting from binding to two epitopes provides a therapeutic benefit. In a particular example, epitopes may be targeted which are present individually on normal cell types, but present together only on abnormal or diseased cells, such as tumor cells. In such a situation, only the abnormal or tumor diseased cells are effectively targeted by the bispecifc ligands according to the invention. Ligand specific for multiple copies of the same epitope, or adjacent epitopes, on the same target (known as chelating dAbs) may also be trimeric or polymeric (tertrameric or more) ligands comprising three, four or more non-complementary binding domains. For example, ligands may be constructed comprising three or four VH domains or VL domains.

Moreover, ligands are provided which bind to multisubunit targets, wherein each binding domain is specific for a subunit of said target. The ligand may be dimeric, trimeric or polymeric. Preferably, the multi-specific ligands according to the above aspects of the invention are obtainable by the method of the first aspect of the invention.

According to the above aspect of the second configuration of the invention, advantageously the first epitope binding domain and the second epitope binding domains are non-complementary immunoglobulin variable domains, as herein defined. That is either VH-VH or VL-VL variable domains.

Chelating dAbs in particular may be prepared according to a preferred aspect of the invention, namely the use of anchor dAbs, in which a library of dimeric, trimeric or multimeric dAbs is constructed using a vector which comprises a constant dAb upstream or downstream of a linker sequence, with a repertoire of second, third and further dAbs being inserted on the other side of the linker. For example, the anchor or guiding dAb may be TAR1-5 (VK), TAR1-27(V), TAR2h-5(VH) or TAR2h-6(VK).

In alternative methodologies, the use of linkers may be avoided, for example by the use of non-covalent bonding or natural affinity between binding domains such as VH and VL. The invention accordingly provides a method for preparing a chelating multimeric ligand comprising the steps of:

(a) providing a vector comprising a nucleic acid sequence encoding a single binding domain specific for a first epitope on a target;

(b) providing a vector encoding a repertoire comprising second binding domains specific for a second epitope on said target, which epitope can be the same or different to the first epitope, said second epitope being adjacent to said first epitope; and

(c) expressing said first and second binding domains; and

(d) isolating those combinations of first and second binding domains which combine together to produce a target-binding dimer.

The first and second epitopes are adjacent such that a multimeric ligand is capable of binding to both epitopes simultaneously. This provides the ligand with the advantages of increased avidity of binding. Where the epitopes are the same, the increased avidity is obtained by the presence of multiple copies of the epitope on the target, allowing at least two copies to be simultaneously bound in order to obtain the increased avidity effect.

The binding domains may be associated by several methods, as well as the use of linkers.

For example, the binding domains may comprise cys residues, avidin and streptavidin groups or other means for non-covalent attachment post- synthesis; those combinations which bind to the target efficiently will be isolated. Alternatively, a linker may be present between the first and second binding domains, which are expressed as a single polypeptide from a single vector, which comprises the first binding domain, the linker and a repertoire of second binding domains, for instance as described above.

In a preferred aspect, the first and second binding domains associate naturally when bound to antigen; for example, VH and VK domains, when bound to adjacent epitopes, will naturally associate in a three-way interaction to form a stable dimer. Such associated proteins can be isolated in a target binding assay. An advantage of this procedure is that only binding domains which bind to closely adjacent epitopes, in the correct conformation, will associate and thus be isolated as a result of their increased avidity for the target.

In an alternative embodiment of the above aspect of the second configuration of the invention, at least one epitope binding domain comprises a non-immunoglobulin ‘protein scaffold’ or ‘protein skeleton’ as herein defined. Suitable non-immunoglobulin protein scaffolds include but are not limited to any of those selected from the group consisting of: SpA, fbronectin, GroEL and other chaperones, lipocallin, CCTLA4 and affibodies, as set forth above.

According to the above aspect of the second configuration of the invention, advantageously, the epitope binding domains are attached to a ‘protein skeleton’.

Advantageously, a protein skeleton according to the invention is an immunoglobulin skeleton. According to the present invention, the term ‘immunoglobulin skeleton’ refers to a protein which comprises at least one immunoglobulin fold and which acts as a nucleus for one or more epitope binding domains, as defined herein.

Preferred “immunoglobulin skeletons” as herein defined includes any one or more of those selected from the following: an immunoglobulin molecule comprising at least (i) the CL (kappa or lambda subclass) domain of an antibody; or (ii) the CH1 domain of an antibody heavy chain; an immunoglobulin molecule comprising the CH1 and CH2 domains of an antibody heavy chain; an immunoglobulin molecule comprising the CH1, CH2 and CH3 domains of an antibody heavy chain; or any of the subset (ii) in conjunction with the CL (kappa or lambda subclass) domain of an antibody. A hinge region domain may also be included. Such combinations of domains may, for example, mimic natural antibodies, such as IgG or IgM, or fragments thereof, such as Fv, scFv, Fab or F(ab′)2 molecules.

Those skilled in the art will be aware that this list is not intended to be exhaustive.

Linking of the skeleton to the epitope binding domains, as herein defined may be achieved at the polypeptide level, that is after expression of the nucleic acid encoding the skeleton and/or the epitope binding domains. Alternatively, the linking step may be performed at the nucleic acid level. Methods of linking a protein skeleton according to the present invention, to the one or more epitope binding domains include the use of protein chemistry and/or molecular biology techniques which will be familiar to those skilled in the art and are described herein.

Advantageously, the closed conformation multispecific ligand may comprise a first domain capable of binding a target molecule, and a second domain capable of binding a molecule or group which extends the half-life of the ligand. For example, the molecule or group may be a bulky agent, such as HSA or a cell matrix protein. As used herein, the phrase “molecule or group which extends the half-life of a ligand” refers to a molecule or chemical group which, when bound by a dual-specific ligand as described herein increases the in vivo half-life of such dual specific ligand when administered to an animal, relative to a ligand that does not bind that molecule or group. Examples of molecules or groups that extend the half- life of a ligand are described hereinbelow. In a preferred embodiment, the closed conformation multispecific ligand may be capable of binding the target molecule only on displacement of the half-life enhancing molecule or group. Thus, for example, a closed conformation multispecific ligand is maintained in circulation in the bloodstream of a subject by a bulky molecule such as HSA. When a target molecule is encountered, competition between the binding domains of the closed conformation multispecific ligand results in displacement of the HSA and binding of the target.

Ligands according to any aspect of the present invention, as well as dAb monomers useful in constructing such ligands, may advantageously dissociate from their cognate 20 target(s) with a K_{d} of 300 nM to 5 pM (ie, 3×10⁻⁷ to 5×10⁻¹² M), preferably 50 nM to 20 pM, or 5 nM to 200 pM or 1 nM to 1OO pM, 1×10⁻⁷ M or less, 1×10⁻⁸ M or less, 1×10⁻⁹ M or less, 1×10⁻¹⁰ M or less, 1×10⁻¹¹ M or less; and/or a Koff rate constant of 5×10⁻¹ to 1×10⁻⁷ S⁻¹, preferably 1×10⁻² to 1×10⁻⁶ S⁻¹, or 5×10⁻³ to 1×10⁻⁵ S⁻¹, or 5×10⁻¹ S⁻¹ or less, or 1×10⁻² S⁻¹ or less, or 1×10⁻³ S⁻¹ or less, or 1×10⁻⁴ S⁻¹ or less, or 1×10⁻⁵ S⁻¹ or less, or 1×10⁻⁶ S¹ or less as determined by surface plasmon resonance. The K_{d} rat

In particular the invention provides an anti-TNF-α dAb monomer (or dual specific ligand comprising such a dAb), homodimer, heterodimer or homotrimer ligand, wherein each dAb binds TNF-α. The ligand binds to TNF-α with a K_{d} of 300 nM to 5 pM (ie, 3×10⁻⁷ to 5×10⁻¹²M), preferably 50 nM to 20 pM, more preferably 5 nM to 200 pM and most preferably 1 nM to 100 pM; expressed in an alternative manner, the K_{d} is 1×10⁻⁷ M or less, preferably 1×10⁻⁸ M or less, more preferably 1×10⁻⁹ M or less, advantageously 1×10⁻¹⁰ M or less and most preferably 1×10⁻¹¹ M or less; and/or a K_{off} rate constant of 5×10⁻¹ to 1×10⁻⁷ S⁻¹, preferably 1×10⁻2 to 1×10⁻⁶ S⁻¹, more preferably 5×10⁻³ to 1×10⁻⁵ S⁻¹, for example 5×10⁻¹S⁻¹ or less, preferably 1×10⁻² S⁻¹ or less, more preferably 1×10^{-less, advantageously} 1×10⁻⁴ S⁻¹ or less, further advantageously 1×10⁻⁵ S⁻¹ or less, and most preferably 1×10⁻⁶ S⁻¹ or less, as determined by surface plasmon resonance.

Preferably, the ligand neutralises TNF-α in a standard L929 assay with an ND50 of 500 nM to 50 pM, preferably or 100 nM to 50 pM, advantageously 10 nM to 100 pM, more preferably 1 nM to 100 pM; for example 50 nM or less, preferably 5 nM or less, advantageously 500 pM or less, more preferably 200 pM or less and most preferably 100 pM or less.

Preferably, the ligand inhibits binding of TNF-α to TNF-α Receptor I (p55 receptor) with an IC50 of 500 nM to 50 pM, preferably 100 nM to 50 pM, more preferably 5 10 nM to 100 pM, advantageously 1 nM to 100 pM; for example 50 nM or less, preferably 5 nM or less, more preferably 500 pM or less, advantageously 200 pM or less, and most preferably 100 pM or less. Preferably, the TNF-α is Human TNF-α.

Furthermore, the invention provides an anti-TNF Receptor I dAb monomer, or dual specific ligand comprising such a dAb, that binds to TNF Receptor I with a K_{d} of 300 nM to 5 pM (ie, 3×10⁻⁷ to 5×10⁻¹²M), preferably 50 nM to 20 pM, more preferably 5 nM to 200 pM and most preferably 1 nM to 100 pM, for example 1×10⁻⁷ M or less, preferably 1×10⁻⁸ M or less, more preferably 1×10⁻⁹ M or less, advantageously 1×10⁻¹⁰ M or less and most preferably 1×10⁻¹¹ M or less; and/or a K_{off} rate constant of 5×10⁻¹ to 1×10⁻⁷ S⁻¹, preferably 1×10⁻2 to 1×10⁻⁶ S⁻¹, more preferably 5×10⁻³ to 1×10⁻⁵ S⁻¹,for example 5×10⁻¹S⁻¹ or less, preferably 1×10⁻² S⁻¹ or less, more preferably 1×10^{-less, advantageously} 1×10⁻⁴ S⁻¹ or less, further advantageously 1×10⁻⁵ S⁻¹ or less, and most preferably 1×10⁻⁶ S⁻¹ or less, as determined by surface plasmon resonance.

Preferably, the dAb monomer or ligand neutralises TNF-α in a standard assay (eg, the L929 or HeLa assays described herein) with an ND50 of 500 nM to 50 pM, preferably 100 nM to 50 pM, more preferably 10 nM to 100 pM, advantageously 1 nM to 100 pM; for example 50 nM or less, preferably 5 nM or less, more preferably 500 pM or less, advantageously 200 pM or less, and most preferably 100 pM or less.

Preferably, the dAb monomer or ligand inhibits binding of TNF-α to TNF-α 5 Receptor I (p55 receptor) with an IC50 of 500 nM to 50 pM, preferably 100 nM to 50 pM, more preferably 10 nM to 100 pM, advantageously 1 nM to 100 pM; for example 50 nM or less, preferably 5 nM or less, more preferably 500 pM or less, advantageously 200 pM or less, and most preferably 100 pM or less. Preferably, the TNF Receptor I target is Human TNF-α.

Furthermore, the invention provides a dAb monomer(or dual specific ligand comprising such a dAb) that binds to serum albumin (SA) with a K_{d} of 1 nM to 500 μM (ie, 1×10⁻⁹ to 5×10⁻⁴), preferably 100 nM to 10,uM. Preferably, for a dual specific ligand comprising a first anti-SA dAb and a second dAb to another target, the affinity (eg Kd and/or Koff as measured by surface plasmon resonance, eg using BiaCore) of the second dAb for its target is from 1 to 100000 times (preferably 100 to 100000, more preferably 1000 to 100000, or 10000 to 100000 times) the affinity of the first dAb for SA. For example, the first dAb binds SA with an affinity of approximately 10 μM, while the second dAb binds its target with an affinity of 100 pM. Preferably, the serum albumin is human serum albumin (HSA).

In one embodiment, the first dAb (or a dAb monomer) binds SA (eg, HSA) with a K_{d} of approximately 50, preferably 70, and more preferably 100, 150 or 200 nM.

The invention moreover provides dimers, trimers and polymers of the aforementioned dAb monomers, in accordance with the foregoing aspect of the present invention.

Ligands according to the invention, including dAb monomers, dimers and trimers, can be linked to an antibody Fc region, comprising one or both of CH2 and CH3 domains, and optionally a hinge region. For example, vectors encoding ligands linked as a single nucleotide sequence to an Fc region may be used to prepare such polypeptides.

In a further aspect of the second configuration of the invention, the present invention provides one or more nucleic acid molecules encoding at least a multispecific ligand as herein defined. In one embodiment, the ligand is a closed conformation ligand. In another embodiment, it is an open conformation ligand. The multispecific ligand may be s encoded on a single nucleic acid molecule; alternatively, each epitope binding domain may be encoded by a separate nucleic acid molecule. Where the ligand is encoded by a single nucleic acid molecule, the domains may be expressed as a fusion polypeptide, or may be separately expressed and subsequently linked together, for example using chemical linking agents. Ligands expressed from separate nucleic acids will be linked together by appropriate means.

The nucleic acid may further encode a signal sequence for export of the polypeptides from a host cell upon expression and may be fused with a surface component of a filamentous bacteriophage particle (or other component of a selection display system) upon expression. Leader sequences, which may be used in bacterial expression and/or phage or phagemid display, include pelB, stH, ompA, phoA, bla and pelA.

In a further aspect of the second configuration of the invention the present invention provides a vector comprising nucleic acid according to the present invention.

In a yet further aspect, the present invention provides a host cell transfected with a vector according to the present invention.

Expression from such a vector may be configured to produce, for example on the surface of a bacteriophage particle, epitope binding domains for selection. This allows selection of displayed domains and thus selection of ‘multispecific ligands’ using the method of the present invention.

In a preferred embodiment of the second configuration of the invention, the epitope binding domains are immunoglobulin variable regions and are selected from single domain V gene repertoires. Generally the repertoire of single antibody domains is displayed on the surface of filamentous bacteriophage. In a preferred embodiment each single antibody domain is selected by binding of a phage repertoire to antigen.

The present invention further provides a kit comprising at least a multispecific ligand according to the present invention, which may be an open conformation or closed conformation ligand. Kits according to the invention may be, for example, diagnostic kits, therapeutic kits, kits for the detection of chemical or biological species, and the like.

In further aspect still of the second configuration of the invention, the present invention provides a homogeneous immunoassay using a ligand according to the present invention.

In a further aspect still of the second configuration of the invention, the present invention provides a composition comprising a closed conformation multispecific ligand, obtainable by a method of the present invention, and a pharmaceutically acceptable carrier, diluent or excipient. Moreover, the present invention provides a method for the treatment of disease using a closed conformation multispecific ligand' or a composition according to the present invention. In a preferred embodiment of the invention the disease is cancer or an inflammatory disease, e.g. rheumatoid arthritis, asthma or Crohn's disease.

In a further aspect of the second configuration of the invention, the present invention provides a method for the diagnosis, including diagnosis of disease using a closed conformation multispecific ligand, or a composition according to the present invention.

Thus in general the binding of an analyte to a closed conformation multispecific ligand may be exploited to displace an agent, which leads to the generation of a signal on displacement. For example, binding of analyte (second antigen) could displace an enzyme (first antigen) bound to the antibody providing the basis for an immunoassay, especially if the enzyme were held to the antibody through its active site.

Thus in a final aspect of the second configuration, the present invention provides a method for detecting the presence of a target molecule, comprising:

(a) providing a closed conformation multispecifc ligand bound to an agent, said ligand being specific for the target molecule and the agent, wherein the agent which is bound by the ligand leads to the generation of a detectable signal on displacement from the ligand; (b) exposing the closed conformation multispecific ligand to the target molecule; and (c) detecting the signal generated as a result of the displacement of the agent.

According to the above aspect of the second configuration of the invention, advantageously, the agent is an enzyme, which is inactive when bound by the closed conformation multi-specific ligand. Alternatively, the agent may be any one or more selected from the group consisting of the following: the substrate for an enzyme, and a fluorescent, luminescent or chromogenic molecule which is inactive or quenched when bound by the ligand.

Sequences similar or homologous (e.g., at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., very high stringency hybridization conditions), to the complement of the strand. The nucleic acids may be present in whole cells, in a cell lysate, or in a partially purified or substantially pure form.

Calculations of “homology” or “sequence identity” or “similarity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes).

In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences.

Advantageously, the BLAST algorithm (version 2.0) is employed for sequence alignment, with parameters set to default values. The BLAST algorithm is described in detail at the world wide web site (“www”) of the National Center for Biotechnology Information (“.ncbi”) of the National Institutes of Health (“nib”) of the U.S. government (“.gov”), in the “/Blast!” directory, in the “blast_help.html” file. The search parameters are defined as follows, and are advantageously set to the defined default parameters.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul, 1990, 20 Proc. Natl. Acad. Sci. USA 87(6):2264-8 (see the “blast_help.html” file, as described above) with a few enhancements. The BLAST programs were tailored for sequence similarity searching, for example to identify homologues to a query sequence. The programs are not generally useful for motif-style searching. For a discussion of basic issues in similarity searching of sequence databases, see Altschul et al. (1994).

The five BLAST programs available at the National Center for Biotechnology Information web site perform the following tasks: “blastp” compares an amino acid query sequence against a protein sequence database; “blastn” compares a nucleotide query sequence against a nucleotide sequence database; “blastx” compares the six-frame conceptual translation products of a nucleotide query sequence (both strands) against a protein sequence database; “tblastn” compares a protein query sequence against a nucleotide sequence database dynamically translated in all six reading frames (both strands). “tblastx” compares the six-frame translations of a nucleotide query sequence against the six-frame translations of a nucleotide sequence database.

BLAST uses the following search parameters:

HISTOGRAM Display a histogram of scores for each search; default is yes. (See s parameter H in the BLAST Manual).

DESCRIPTIONS Restricts the number of short descriptions of matching sequences reported to the number specified; default limit is 100 descriptions. (See parameter V in the manual page). See also EXPECT and CUTOFF.

ALIGNMENTS Restricts database sequences to the number specified for which high scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

EXPECT The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

MATRIX Specify an alternate scoring matrix for BLASTP, BLASTX, TBLASTN and TBLASTX. The default matrix is BLOSUM62 (Henikoff & Henikoff, 1992, Proc. Natl. 30 Acad. Sci. USA 89(22):10915-9). The valid alternative choices include: PAM40, PAM120, PAM:250 and IDENTITY. No alternate scoring matrices are available for BLASTN; specifying the MATRIX directive in BLASTN requests returns an error response.

STRAND Restrict a TBLASTN search to just the top or bottom strand of the database sequences; or restrict a BLASTN, BLASTX or TBLASTX search to just reading frames on the top or bottom strand of the query sequence.

FILTER Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States, 1993, Computers and Chemistry 17:191-201, or, for BLASTN, by the DUST program of Tatusov and Lipman (see the world wide web site of the NCBI). Filtering can eliminate statistically significant but biologically uninteresting reports from the blast output (e.g., hits against common acidic-, basic- or proline-rich regions), leaving the more biologically interesting regions of the query sequence available for specific matching against database sequences. Low complexity sequence found by a filter program is substituted using the letter “N” in nucleotide sequence (e.g., “N” repeated 13 times) and the letter “X” in protein sequences (e.g., “X” repeated 9 times).

Filtering is only applied to the query sequence (or its translation products), not to database sequences. Default filtering is DUST for BLASTN, SEG for other programs. It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi Causes NCBI gi identifiers to be shown in the output, in addition to the accession and/or locus name.

Most preferably, sequence comparisons are conducted using the simple BLAST search algorithm provided at the NCBI world wide web site described above, in the “/BLAST” directory.

### Preparation of Immunoglobulin Based Multi-Specific Ligands

Dual specific ligands according to the invention, whether open or closed in conformation according to the desired configuration of the invention, may be prepared according to previously established techniques, used in the field of antibody engineering, for the preparation of scFv, “phage” antibodies and other engineered antibody molecules. Techniques for the preparation of antibodies, and in particular bispecific antibodies, are for example described in the following reviews and the references cited therein: Winter & Milstein, (1991) Nature 349:293-299; Plueckthun (1992) Immunological Reviews 130:151-188; Wright et al., (1992) Crti. Rev. Immunol. 12:125-168; Holliger, P. & Winter, G. (1993) Curr. Op. Biotechn. 4, 446-449; Carter, et al. (1995) J. Hematother. 4, 463-470; Chester, K. A. & Hawkins, R. E. (1995) Trends Biotechn. 13, 294-300; Hoogenboom, H. R. (1997) Nature Biotechnol. 15, 125-126; Fearon, D. (1997) Nature Biotechnol. 15, 618-619; Plückthun, A. & Pack, P. (1997) Immunotechnology 3, 83-105; Carter, P. & Merchant, A. M. (1997) Curr. Opin. Biotechnol. 8, 449-454; Holliger, P. & Winter, G. (1997) Cancer Immunol. Immunother. 45,128-130.

The invention provides for the selection of variable domains against two different antigens or epitopes, and subsequent combination of the variable domains.

The techniques employed for selection of the variable domains employ libraries and selection procedures which are known in the art. Natural libraries (Marks et al. (1991) J. Mol. Biol., 222: 581; Vaughan et al. (1996) Nature Biotech., 14: 309) which use rearranged V genes harvested from human B cells are well known to those skilled in the art. Synthetic libraries (Hoogenboom & Winter (1992) J. Mol. Biol., 227: 381; Barbas et al. (1992) Proc. Natl. Acad. Sci. USA, 89: 4457; Nissim et al. (1994) EMBO J., 13: 692; Griffiths et al. (1994) EMBO J., 13: 3245; De Kruif et al. (1995) J. Mol. Biol., 248: 97) are prepared by cloning immunoglobulin V genes, usually using PCR. Errors in the PCR process can lead to a high degree of randomisation. V_{H} and/or V_{L} libraries may be selected against target antigens or epitopes separately, in which case single domain binding is directly selected for, or together.

A preferred method for making a dual specific ligand according to the present invention comprises using a selection system in which a repertoire of variable domains is selected for binding to a first antigen or epitope and a repertoire of variable domains is selected for binding to a second antigen or epitope. The selected variable first and second variable domains are then combined and the dual-specific ligand selected for binding to both first and second antigen or epitope. Closed conformation ligands are selected for binding both first and second antigen or epitope in isolation but not simultaneously.

### A. Library Vector Systems

A variety of selection systems are known in the art which are suitable for use in the present invention. Examples of such systems are described below.

Bacteriophage lambda expression systems may be screened directly as bacteriophage plaques or as colonies of lysogens, both as previously described (Huse et al. (1989) Science, 246: 1275; Caton and Koprowski (1990) Proc. Natl. Acad. Sci. U.S.A., 87; Mullinax et al. (1990) Proc. Natl. Acad. Sci. U.S.A., 87: 8095; Persson et al. (1991) Proc. Natl. Acad. Sci. U.S.A., 88: 2432) and are of use in the invention. Whilst such expression systems can be used to screen up to 10⁶ different members of a library, they are not really suited to screening of larger numbers (greater than 10⁶ members).

Of particular use in the construction of libraries are selection display systems, which enable a nucleic acid to be linked to the polypeptide it expresses. As used herein, a selection display system is a system that permits the selection, by suitable display means, of the individual members of the library by binding the generic and/or target ligands.

Selection protocols for isolating desired members of large libraries are known in the art, as typified by phage display techniques. Such systems, in which diverse peptide sequences are displayed on the surface of filamentous bacteriophage (Scott and Smith (1990) Science, 249: 386), have proven useful for creating libraries of antibody fragments (and the nucleotide sequences that encoding them) for the in vitro selection and amplification of specific antibody fragments that bind a target antigen (McCafferty et al., WO 92/01047). The nucleotide sequences encoding the V_{H} and V_{L} regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phagebodies). An advantage of phage-based display systems is that, because they are biological systems, selected library members can be amplified simply by growing the phage containing the selected library member in bacterial cells. Furthermore, since the nucleotide sequence that encode the polypeptide library member is contained on a phage or phagemid vector, sequencing, expression and subsequent genetic manipulation is relatively straightforward.

Methods for the construction of bacteriophage antibody display libraries and lambda phage expression libraries are well known in the art (McCafferty et al. (1990) Nature, 348: 552; Kang et al. (1991) Proc. Natl. Acad. Sci. USA., 88: 4363; Clackson et al. (1991) Nature, 352: 624; Lowman et al. (1991) Biochemistry, 30: 10832; Burton et al. (1991) Proc. Natl. Acad. Sci USA., 88: 10134; Hoogenboom et al. (1991) Nucleic Acids Res., 19: 4133; Chang et al. (1991) J. Immunol., 147: 3610; Breitling et al. (1991) Gene, 104: 147; Marks et al. (1991) supra; Barbas et al. (1992) supra; Hawkins and Winter (1992) J. Immunol., 22: 867; Marks et al., 1992, J. Biol. Chem., 267: 16007; Lerner et al. (1992) Science, 258: 1313, incorporated herein by reference).

One particularly advantageous approach has been the use of scFv phage-libraries (Huston et al., 1988, Proc. Natl. Acad. Sci U.S.A., 85: 5879-5883; Chaudhary et al. (1990) Proc. Natl. Acad. Sci U.S.A., 87: 1066-1070; McCafferty et al. (1990) supra; Clackson et al. (1991) Nature, 352: 624; Marks et al. (1991) J. Mol. Biol., 222: 581; Chiswell et al. (1992) Trends Biotech., 10: 80; Marks et al. (1992) J. Biol. Chem., 267). Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys), which are incorporated herein by reference.

Other systems for generating libraries of polypeptides involve the use of cell-free enzymatic machinery for the in vitro synthesis of the library members. In one method, RNA molecules are selected by alternate rounds of selection against a target ligand and PCR amplification (Tuerk and Gold (1990) Science, 249: 505; Ellington and Szostak (1990) Nature, 346: 818). A similar technique may be used to identify DNA sequences which bind a predetermined human transcription factor (Thiesen and Bach (1990) Nucleic Acids Res., 18: 3203; Beaudry and Joyce (1992) Science, 257: 635; WO92/05258 and WO92/14843). In a similar way, in vitro translation can be used to synthesise polypeptides as a method for generating large libraries. These methods which generally comprise stabilised polysome complexes, are described further in WO88/08453, WO90/05785, WO90/07003, WO91/02076, WO91/05058, and WO92/02536. Alternative display systems which are not phage-based, such as those disclosed in WO95/22625 and WO95/11922 (Affymax) use the polysomes to display polypeptides for selection.

A still further category of techniques involves the selection of repertoires in artificial compartments, which allow the linkage of a gene with its gene product. For example, a selection system in which nucleic acids encoding desirable gene products may be selected in microcapsules formed by water-in-oil emulsions is described in WO99/02671, WO00/40712 and Tawfik & Griffiths (1998) Nature Biotechnol 16(7), 652-6. Genetic elements encoding a gene product having a desired activity are compartmentalised into microcapsules and then transcribed and/or translated to produce their respective gene products (RNA or protein) within the microcapsules. Genetic elements which produce gene product having desired activity are subsequently sorted. This approach selects gene products of interest by detecting the desired activity by a variety of means.

### B. Library Construction.

Libraries intended for selection, may be constructed using techniques known in the art, for example as set forth above, or may be purchased from commercial sources. Libraries which are useful in the present invention are described, for example, in WO99/20749. Once a vector system is chosen and one or more nucleic acid sequences encoding polypeptides of interest are cloned into the library vector, one may generate diversity within the cloned molecules by undertaking mutagenesis prior to expression; alternatively, the encoded proteins may be expressed and selected, as described above, before mutagenesis and additional rounds of selection are performed. Mutagenesis of nucleic acid sequences encoding structurally optimised polypeptides is carried out by standard molecular methods. Of particular use is the polymerase chain reaction, or PCR, (Mullis and Faloona (1987) Methods Enzymol., 155: 335, herein incorporated by reference). PCR, which uses multiple cycles of DNA replication catalysed by a thermostable, DNA-dependent DNA polymerase to amplify the target sequence of interest, is well known in the art. The construction of various antibody libraries has been discussed in Winter et al. (1994) Ann. Rev. Immunology 12, 433-55, and references cited therein.

PCR is performed using template DNA (at least 1 fg; more usefully, 1-1000 ng) and at least 25 pmol of oligonucleotide primers; it may be advantageous to use a larger amount of primer when the primer pool is heavily heterogeneous, as each sequence is represented by only a small fraction of the molecules of the pool, and amounts become limiting in the later amplification cycles. A typical reaction mixture includes: 2 μl of DNA, 25 pmol of oligonucleotide primer, 2.5 μl of 10×PCR buffer 1 (Perkin-Elmer, Foster City, Calif.), 0.4 μl of 1.25 μM dNTP, 0.15 μl (or 2.5 units) of Taq DNA polymerase (Perkin Elmer, Foster City, Calif.) and deionized water to a total volume of 25 μl. Mineral oil is overlaid and the PCR is performed using a programmable thermal cycler. The length and temperature of each step of a PCR cycle, as well as the number of cycles, is adjusted in accordance to the stringency requirements in effect. Annealing temperature and timing are determined both by the efficiency with which a primer is expected to anneal to a template and the degree of mismatch that is to be tolerated; obviously, when nucleic acid molecules are simultaneously amplified and mutagenised, mismatch is required, at least in the first round of synthesis. The ability to optimise the stringency of primer annealing conditions is well within the knowledge of one of moderate skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1-5 minutes, depending on the length of the amplified product). Final extension is generally for 4 minutes at 72° C., and may be followed by an indefinite (0-24 hour) step at 4° C.

C. Combining Single Variable Domains

Domains useful in the invention, once selected, may be combined by a variety of methods known in the art, including covalent and non-covalent methods.

Preferred methods include the use of polypeptide linkers, as described, for example, in connection with scFv molecules (Bird et al., (1988) Science 242:423-426). Discussion of suitable linkers is provided in Bird et al. Science 242, 423-426; Hudson et al , Journal Immunol Methods 231 (1999) 177-189; Hudson et al, Proc Nat Acad Sci USA 85, 5879-5883. Linkers are preferably flexible, allowing the two single domains to interact. One linker example is a (Gly₄ Ser)ₙ linker, where n=1 to 8, eg, 2, 3, 4, 5 or 7. The linkers used in diabodies, which are less flexible, may also be employed (Holliger et al., (1993) PNAS (USA) 90:6444-6448).

In one embodiment, the linker employed is not an immunoglobulin hinge region.

Variable domains may be combined using methods other than linkers. For example, the use of disulphide bridges, provided through naturally-occurring or engineered cysteine residues, may be exploited to stabilise V_{H}-V_{H}, V_{L}-V_{L} or V_{H}-V_{L} dimers (Reiter et al., (1994) Protein Eng. 7:697-704) or by remodelling the interface between the variable domains to improve the “fit” and thus the stability of interaction (Ridgeway et al., (1996) Protein Eng. 7:617-621; Zhu et al., (1997) Protein Science 6:781-788).

Other techniques for joining or stabilising variable domains of immunoglobulins, and in particular antibody V_{H} domains, may be employed as appropriate.

In accordance with the present invention, dual specific ligands can be in “closed” conformations in solution. A “closed” configuration is that in which the two domains (for example V_{H} and V_{L}) are present in associated form, such as that of an associated V_{H}-V_{L} pair which forms an antibody binding site. For example, scFv may be in a closed conformation, depending on the arrangement of the linker used to link the V_{H} and V_{L} domains. If this is sufficiently flexible to allow the domains to associate, or rigidly holds them in the associated position, it is likely that the domains will adopt a closed conformation.

Similarly, V_{H} domain pairs and V_{L} domain pairs may exist in a closed conformation. Generally, this will be a function of close association of the domains, such as by a rigid linker, in the ligand molecule. Ligands in a closed conformation will be unable to bind both the molecule which increases the half-life of the ligand and a second target molecule. Thus, the ligand will typically only bind the second target molecule on dissociation from the molecule which increases the half-life of the ligand.

Moreover, the construction of V_{H}/V_{H}, V_{L}/V_{L} or V_{H}/V_{L} dimers without linkers provides for competition between the domains.

Ligands according to the invention may moreover be in an open conformation. In such a conformation, the ligands will be able to simultaneously bind both the molecule which increases the half-life of the ligand and the second target molecule. Typically, variable domains in an open configuration are (in the case of V_{H}-V_{L} pairs) held far enough apart for the domains not to interact and form an antibody binding site and not to compete for binding to their respective epitopes. In the case of V_{H}/V_{H} or V_{L}/V_{L} dimers, the domains are not forced together by rigid linkers. Naturally, such domain pairings will not compete for antigen binding or form an antibody binding site.

Fab fragments and whole antibodies will exist primarily in the closed conformation, although it will be appreciated that open and closed dual specific ligands are likely to exist in a variety of equilibria under different circumstances. Binding of the ligand to a target is likely to shift the balance of the equilibrium towards the open configuration. Thus, certain ligands according to the invention can exist in two conformations in solution, one'of which (the open form) can bind two antigens or epitopes independently, whilst the alternative conformation (the closed form) can only bind one antigen or epitope; antigens or epitopes thus compete for binding to the ligand in this conformation.

Although the open form of the dual specific ligand may thus exist in equilibrium with the closed form in solution, it is envisaged that the equilibrium will favour the closed form; moreover, the open form can be sequestered by target binding into a closed conformation. Preferably, therefore, certain dual specific ligands of the invention are present in an equilibrium between two (open and closed) conformations.

Dual specific ligands according to the invention may be modified in order to favour an open or closed conformation. For example, stabilisation of V_{H}-V_{L} interactions with disulphide bonds stabilises the closed conformation. Moreover, linkers used to join the domains, including V_{H} domain and V_{L} domain pairs, may be constructed such that the open from is favoured; for example, the linkers may sterically hinder the association of the domains, such as by incorporation of large amino acid residues in opportune locations, or the designing of a suitable rigid structure which will keep the domains physically spaced apart.

### D. Characterisation of the Dual-Specific Ligand.

The binding of the dual-specific ligand to its specific antigens or epitopes can be tested by methods which will be familiar to those skilled in the art and include ELISA. In a preferred embodiment of the invention binding is tested using monoclonal phage ELISA.

Phage ELISA may be performed according to any suitable procedure: an exemplary protocol is set forth below.

Populations of phage produced at each round of selection can be screened for binding by ELISA to the selected antigen or epitope, to identify “polyclonal” phage antibodies. Phage from single infected bacterial colonies from these populations can then be screened by ELISA to identify “monoclonal” phage antibodies. It is also desirable to screen soluble antibody fragments for binding to antigen or epitope, and this can also be undertaken by ELISA using reagents, for example, against a C- or N-terminal tag (see for example Winter et al. (1994) Ann. Rev. Immunology 12, 433-55 and references cited therein.

The diversity of the selected phage monoclonal antibodies may also be assessed by gel electrophoresis of PCR products (Marks et al. 1991, supra; Nissim et al. 1994 supra), probing (Tomlinson et al., 1992) J. Mol. Biol. 227, 776) or by sequencing of the vector DNA.

### E. Structure of ‘Dual-Specific Ligands’.

As described above, an antibody is herein defined as an antibody (for example IgG, IgM, IgA, IgA, IgE) or fragment (Fab, Fv, disulphide linked Fv, scFv, diabody) which comprises at least one heavy and a light chain variable domain, at least two heavy chain variable domains or at least two light chain variable domains. It may be at least partly derived from any species naturally producing an antibody, or created by recombinant DNA technology; whether isolated from serum, B-cells, hybridomas, transfectomas, yeast or bacteria).

In a preferred embodiment of the invention the dual-specific ligand comprises at least one single heavy chain variable domain of an antibody and one single light chain variable domain of an antibody, or two single heavy or light chain variable domains. For example, the ligand may comprise a V_{H}/V_{L} pair, a pair of V_{H} domains or a pair of V_{L} domains.

The first and the second variable domains of such a ligand may be on the same polypeptide chain. Alternatively they may be on separate polypeptide chains. In the case that they are on the same polypeptide chain they may be linked by a linker, which is preferentially a peptide sequence, as described above.

The first and second variable domains may be covalently or non-covalently associated. In the case that they are covalently associated, the covalent bonds may be disulphide bonds.

In the case that the variable domains are selected from V-gene repertoires selected for instance using phage display technology as herein described, then these variable domains comprise a universal framework region, such that is they may be recognised by a specific generic ligand as herein defined. The use of universal frameworks, generic ligands and the like is described in WO99/20749.

Where V-gene repertoires are used variation in polypeptide sequence is preferably located within the structural loops of the variable domains. The polypeptide sequences of either variable domain may be altered by DNA shuffling or by mutation in order to enhance the interaction of each variable domain with its complementary pair. DNA shuffling is known in the art and taught, for example, by Stemmer, 1994, Nature 370: 389-391 and U.S. Pat. No. 6,297,053, both of which are incorporated herein by reference. Other methods of mutagenesis are well known to those of skill in the art.

In a preferred embodiment of the invention the ‘dual-specific ligand’ is a single chain Fv fragment. In an alternative embodiment of the invention, the ‘dual-specific ligand’ consists of a Fab format.

In a further aspect, the present invention provides nucleic acid encoding at least a ‘dual-specific ligand’ as herein defined.

One skilled in the art will appreciate that, depending on the aspect of the invention, both antigens or epitopes may bind simultaneously to the same antibody molecule. Alternatively, they may compete for binding to the same antibody molecule. For example, where both epitopes are bound simultaneously, both variable domains of a dual specific ligand are able to independently bind their target epitopes. Where the domains compete, the one variable domain is capable of binding its target, but not at the same time as the other variable domain binds its cognate target; or the first variable domain is capable of binding its target, but not at the same time as the second variable domain binds its cognate target.

The variable domains may be derived from antibodies directed against target antigens or epitopes. Alternatively they may be derived from a repertoire of single antibody domains such as those expressed on the surface of filamentous bacteriophage. Selection may be performed as described below.

In general, the nucleic acid molecules and vector constructs required for the performance of the present invention may be constructed and manipulated as set forth in standard laboratory manuals, such as Sambrook et al. (1989) Molecular Cloning: A Laboratory Manual, Cold Spring Harbor, USA.

The manipulation of nucleic acids useful in the present invention is typically carried out in recombinant vectors.

Thus in a further aspect, the present invention provides a vector comprising nucleic acid encoding at least a ‘dual-specific ligand’ as herein defined.

As used herein, vector refers to a discrete element that is used to introduce heterologous DNA into cells for the expression and/or replication thereof. Methods by which to select or construct and, subsequently, use such vectors are well known to one of ordinary skill in the art. Numerous vectors are publicly available, including bacterial plasmids, bacteriophage, artificial chromosomes and episomal vectors. Such vectors may be used for simple cloning and mutagenesis; alternatively gene expression vector is employed. A vector of use according to the invention may be selected to accommodate a polypeptide coding sequence of a desired size, typically from 0.25 kilobase (kb) to 40 kb or more in length A suitable host cell is transformed with the vector after in vitro cloning manipulations. Each vector contains various functional components, which generally include a cloning (or “polylinker”) site, an origin of replication and at least one selectable marker gene. If given vector is an expression vector, it additionally possesses one or more of the following: enhancer element, promoter, transcription termination and signal sequences, each positioned in the vicinity of the cloning site, such that they are operatively linked to the gene encoding a ligand according to the invention.

Both cloning and expression vectors generally contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. The origin of replication from the plasmid pBR322 is suitable for most Gram-negative bacteria, the 2 micron plasmid origin is suitable for yeast, and various viral origins (e.g. SV 40, adenovirus) are useful for cloning vectors in mammalian cells. Generally, the origin of replication is not needed for mammalian expression vectors unless these are used in mammalian cells able to replicate high levels of DNA, such as COS cells.

Advantageously, a cloning or expression vector may contain a selection gene also referred to as selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available in the growth media.

Since the replication of vectors encoding a ligand according to the present invention is most conveniently performed in E. coli, an E. coli-selectable marker, for example, the β-lactamase gene that confers resistance to the antibiotic ampicillin, is of use. These can be obtained from E. coli plasmids, such as pBR322 or a pUC plasmid such as pUC18 or pUC19.

Expression vectors usually contain a promoter that is recognised by the host organism and is operably linked to the coding sequence of interest. Such a promoter may be inducible or constitutive. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the β-lactamase and lactose promoter systems, alkaline phosphatase, the tryptophan (trp) promoter system and hybrid promoters such as the tac promoter. Promoters for use in bacterial systems will also generally contain a Shine-Delgarno sequence operably linked to the coding sequence.

The preferred vectors are expression vectors that enables the expression of a nucleotide sequence corresponding to a polypeptide library member. Thus, selection with the first and/or second antigen or epitope can be performed by separate propagation and expression of a single clone expressing the polypeptide library member or by use of any selection display system. As described above, the preferred selection display system is bacteriophage display. Thus, phage or phagemid vectors may be used, eg pIT1 or pIT2. Leader sequences useful in the invention include pelB, stII, ompA, phoA, bla and pelA. One example are phagemid vectors which have an E. coli. origin of replication (for double stranded replication) and also a phage origin of replication (for production of single-stranded DNA). The manipulation and expression of such vectors is well known in the art (Hoogenboom and Winter (1992) supra; Nissim et al. (1994) supra). Briefly, the vector contains a β-lactamase gene to confer selectivity on the phagemid and a lac promoter upstream of a expression cassette that consists (N to C terminal) of a pelB leader sequence (which directs the expressed polypeptide to the periplasmic space), a multiple cloning site (for cloning the nucleotide version of the library member), optionally, one or more peptide tag (for detection), optionally, one or more TAG stop codon and the phage protein pIII. Thus, using various suppressor and non-suppressor strains of E. coli and with the addition of glucose, iso-propyl thio-β-D-galactoside (IPTG) or a helper phage, such as VCS M13, the vector is able to replicate as a plasmid with no expression, produce large quantities of the polypeptide library member only or produce phage, some of which contain at least one copy of the polypeptide-pIII fusion on their surface.

Construction of vectors encoding ligands according to the invention employs conventional ligation techniques. Isolated vectors or DNA fragments are cleaved, tailored, and religated in the form desired to generate the required vector. If desired, analysis to confirm that the correct sequences are present in the constructed vector can be performed in a known fashion. Suitable methods for constructing expression vectors, preparing in vitro transcripts, introducing DNA into host cells, and performing analyses for assessing expression and function are known to those skilled in the art. The presence of a gene sequence in a sample is detected, or its amplification and/or expression quantified by conventional methods, such as Southern or Northern analysis, Western blotting, dot blotting of DNA, RNA or protein, in situ hybridisation, immunocytochemistry or sequence analysis of nucleic acid or protein molecules. Those skilled in the art will readily envisage how these methods may be modified, if desired.

### Structure of Closed Conformation Multispecific Ligands

According to one aspect of the second configuration of the invention present invention, the two or more non-complementary epitope binding domains are linked so that they are in a closed conformation as herein defined. Advantageously, they may be further attached to a skeleton which may, as an alternative, or in addition to a linker described herein, facilitate the formation and/or maintenance of the closed conformation of the epitope binding sites with respect to one another.

### (I) Skeletons

Skeletons may be based on immunoglobulin molecules or may be non-immunoglobulin in origin as set forth above. Preferred immunoglobulin skeletons as herein defined includes any one or more of those selected from the following: an immunoglobulin molecule comprising at least (i) the CL (kappa or lambda subclass) domain of an antibody; or (ii) the CH1 domain of an antibody heavy chain; an immunoglobulin molecule comprising the CH1 and CH2 domains of an antibody heavy chain; an immunoglobulin molecule comprising the CH1, CH2 and CH3 domains of an antibody heavy chain; or any of the subset (ii) in conjunction with the CL (kappa or lambda subclass) domain of an antibody. A hinge region domain may also be included.. Such combinations of domains may, for example, mimic natural antibodies, such as IgG or IgM, or fragments thereof, such as Fv, scFv, Fab or F(ab′)₂ molecules. Those skilled in the art will be aware that this list is not intended to be exhaustive.

### (II) Protein Scaffolds

Each epitope binding domain comprises a protein scaffold and one or more CDRs which are involved in the specific interaction of the domain with one or more epitopes. Advantageously, an epitope binding domain according to the present invention comprises three CDRs. Suitable protein scaffolds include any of those selected from the group consisting of the following: those based on immunoglobulin domains, those based on fibronectin, those based on affibodies, those based on CTLA4, those based on chaperones such as GroEL, those based on lipocallin and those based on the bacterial Fc receptors SpA and SpD. Those skilled in the art will appreciate that this list is not intended to be exhaustive.

### F: Scaffolds for Use in Constructing Dual Specific Ligands

i. Selection of the Main-Chain Conformation

The members of the immunoglobulin superfamily all share a similar fold for their polypeptide chain. For example, although antibodies are highly diverse in terms of their primary sequence, comparison of sequences and crystallographic structures has revealed that, contrary to expectation, five of the six antigen binding loops of antibodies (H1, H2, L1, L2, L3) adopt a limited number of main-chain conformations, or canonical structures (Chothia and Lesk (1987) J. Mol. Biol., 196: 901; Chothia et al. (1989) Nature, 342: 877). Analysis of loop lengths and key residues has therefore enabled prediction of the main-chain conformations of H1, H2, L1, L2 and L3 found in the majority of human antibodies (Chothia et al. (1992) J. Mol. Biol., 227: 799; Tomlinson et al. (1995) EMBO J., 14: 4628; Williams et al. (1996) J. Mol. Biol., 264: 220). Although the H3 region is much more diverse in terms of sequence, length and structure (due to the use of D segments), it also forms a limited number of main-chain conformations for short loop lengths which depend on the length and the presence of particular residues, or types of residue, at key positions in the loop and the antibody framework (Martin et al. (1996) J. Mol. Biol., 263: 800; Shirai et al. (1996) FEBS Letters, 399: 1).

The dual specific ligands of the present invention are advantageously assembled from libraries of domains, such as libraries of V_{H} domains and/or libraries of V_{L} domains. Moreover, the dual specific ligands of the invention may themselves be provided in the form of libraries. In one aspect of the present invention, libraries of dual specific ligands and/or domains are designed in which certain loop lengths and key residues have been chosen to ensure that the main-chain conformation of the members is known. Advantageously, these are real conformations of immunoglobulin superfamily molecules found in nature, to minimise the chances that they are non-functional, as discussed above. Germline V gene segments serve as one suitable basic framework for constructing antibody or T-cell receptor libraries; other sequences are also of use. Variations may occur at a low frequency, such that a small number of functional members may possess an altered main-chain conformation, which does not affect its function.

Canonical structure theory is also of use to assess the number of different main-chain conformations encoded by ligands, to predict the main-chain conformation based on ligand sequences and to chose residues for diversification which do not affect the canonical structure. It is known that, in the human V_{κ} domain, the L1 loop can adopt one of four canonical structures, the L2 loop has a single canonical structure and that 90% of human V_{κ} domains adopt one of four or five canonical structures for the L3 loop (Tomlinson et al. (1995) supra); thus, in the V_{κ} domain alone, different canonical structures can combine to create a range of different main-chain conformations. Given that the V_{λ} domain encodes a different range of canonical structures for the L1, L2 and L3 loops and that V_{κ} and V_{λ} domains can pair with any V_{H} domain which can encode several canonical structures for the H1 and H2 loops, the number of canonical structure combinations observed for these five loops is very large. This implies that the generation of diversity in the main-chain conformation may be essential for the production of a wide range of binding specificities. However, by constructing an antibody library based on a single known main-chain conformation it has been found, contrary to expectation, that diversity in the main-chain conformation is not required to generate sufficient diversity to target substantially all antigens. Even more surprisingly, the single main-chain conformation need not be a consensus structure—a single naturally occurring conformation can be used as the basis for an entire library. Thus, in a preferred aspect, the dual-specific ligands of the invention possess a single known main-chain conformation.

The single main-chain conformation that is chosen is preferably commonplace among molecules of the immunoglobulin superfamily type in question. A conformation is commonplace when a significant number of naturally occurring molecules are observed to adopt it. Accordingly, in a preferred aspect of the invention, the natural occurrence of the different main-chain conformations for each binding loop of an immunoglobulin domain are considered separately and then a naturally occurring variable domain is chosen which possesses the desired combination of main-chain conformations for the different loops. If none is available, the nearest equivalent may be chosen. It is preferable that the desired combination of main-chain conformations for the different loops is created by selecting germline gene segments which encode the desired main-chain conformations. It is more preferable, that the selected germline gene segments are frequently expressed in nature, and most preferable that they are the most frequently expressed of all natural germline gene segments.

In designing dual specific ligands or libraries thereof the incidence of the different main-chain conformations for each of the six antigen binding loops may be considered separately. For H1, H2, L1, L2 and L3, a given conformation that is adopted by between 20% and 100% of the antigen binding loops of naturally occurring molecules is chosen. Typically, its observed incidence is above 35% (i.e. between 35% and 100%) and, ideally, above 50% or even above 65%. Since the vast majority of H3 loops do not have canonical structures, it is preferable to select a main-chain conformation which is commonplace among those loops which do display canonical structures. For each of the loops, the conformation which is observed most often in the natural repertoire is therefore selected. In human antibodies, the most popular canonical structures (CS) for each loop are as follows: H1-CS 1 (79% of the expressed repertoire), H2-CS 3 (46%), L1-CS 2 of V_{κ} (39%), L2-CS 1 (100%), L3-CS 1 of V_{κ} (36%) (calculation assumes a κ:λ ratio of 70:30, Hood et al. (1967) Cold Spring Harbor Symp. Quant. Biol., 48: 133). For H3 loops that have canonical structures, a CDR3 length (Kabat et al. (1991) Sequences of proteins of immunological interest, U.S. Department of Health and Human Services) of seven residues with a salt-bridge from residue 94 to residue 101 appears to be the most common. There are at least 16 human antibody sequences in the EMBL data library with the required H3 length and key residues to form this conformation and at least two crystallographic structures in the protein data bank which can be used as a basis for antibody modelling (2cgr and 1tet). The most frequently expressed germline gene segments that this combination of canonical structures are the V_{H} segment 3-23 (DP-47), the J_{H} segment JH4b, the V_{κ} segment O2/O12 (DPK9) and the J_{κ} segment J_{κ}I. V_{H} segments DP45 and DP38 are also suitable. These segments can therefore be used in combination as a basis to construct a library with the desired single main-chain conformation.

Alternatively, instead of choosing the single main-chain conformation based on the natural occurrence of the different main-chain conformations for each of the binding loops in isolation, the natural occurrence of combinations of main-chain conformations is used as the basis for choosing the single main-chain conformation. In the case of antibodies, for example, the natural occurrence of canonical structure combinations for any two, three, four, five or for all six of the antigen binding loops can be determined. Here, it is preferable that the chosen conformation is commonplace in naturally occurring antibodies and most preferable that it observed most frequently in the natural repertoire. Thus, in human antibodies, for example, when natural combinations of the five antigen binding loops, H1, H2, L1, L2 and L3, are considered, the most frequent combination of canonical structures is determined and then combined with the most popular conformation for the H3 loop, as a basis for choosing the single main-chain conformation.

ii. Diversification of the Canonical Sequence

Having selected several known main-chain conformations or, preferably a single known main-chain conformation, dual specific ligands according to the invention or libraries for use in the invention can be constructed by varying the binding site of the molecule in order to generate a repertoire with structural and/or functional diversity. This means that variants are generated such that they possess sufficient diversity in their structure and/or in their function so that they are capable of providing a range of activities.

The desired diversity is typically generated by varying the selected molecule at one or more positions. The positions to be changed can be chosen at random or are preferably selected. The variation can then be achieved either by randomisation, during which the resident amino acid is replaced by any amino acid or analogue thereof, natural or synthetic, producing a very large number of variants or by replacing the resident amino acid with one or more of a defined subset of amino acids, producing a more limited number of variants.

Various methods have been reported for introducing such diversity. Error-prone PCR (Hawkins et al. (1992) J. Mol. Biol., 226: 889), chemical mutagenesis (Deng et al. (1994) J. Biol. Chem., 269: 9533) or bacterial mutator strains (Low et al. (1996) J. Mol. Biol., 260: 359) can be used to introduce random mutations into the genes that encode the molecule. Methods for mutating selected positions are also well known in the art and include the use of mismatched oligonucleotides or degenerate oligonucleotides, with or without the use of PCR. For example, several synthetic antibody libraries have been created by targeting mutations to the antigen binding loops. The H3 region of a human tetanus toxoid-binding Fab has been randomised to create a range of new binding specificities (Barbas et al. (1992) Proc. Natl. Acad. Sci. USA, 89: 4457). Random or semi-random H3 and L3 regions have been appended to germline V gene segments to produce large libraries with unmutated framework regions (Hoogenboom & Winter (1992) J. Mol. Biol., 227: 381; Barbas et al. (1992) Proc. Natl. Acad. Sci. USA, 89: 4457; Nissim et al. (1994) EMBO J., 13: 692; Griffiths et al. (1994) EMBO J., 13: 3245; De Kruif et al. (1995) J. Mol. Biol., 248: 97). Such diversification has been extended to include some or all of the other antigen binding loops (Crameri et al. (1996) Nature Med., 2: 100; Riechmann et al. (1995) Bio/Technology, 13: 475; Morphosys, WO97/08320, supra).

Since loop randomisation has the potential to create approximately more than 10¹⁵ structures for H3 alone and a similarly large number of variants for the other five loops, it is not feasible using current transformation technology or even by using cell free systems to produce a library representing all possible combinations. For example, in one of the largest libraries constructed to date, 6×10¹⁰ different antibodies, which is only a fraction of the potential diversity for a library of this design, were generated (Griffiths et al. (1994) supra).

In a preferred embodiment, only those residues which are directly involved in creating or modifying the desired function of the molecule are diversified. For many molecules, the function will be to bind a target and therefore diversity should be concentrated in the target binding site, while avoiding changing residues which are crucial to the overall packing of the molecule or to maintaining the chosen main-chain conformation.

### Diversification of the Canonical Sequence as it Applies to Antibody Domains

In the case of antibody dual-specific ligands, the binding site for the target is most often the antigen binding site. Thus, in a highly preferred aspect, the invention provides libraries of or for the assembly of antibody dual-specific ligands in which only those residues in the antigen binding site are varied. These residues are extremely diverse in the human antibody repertoire and are known to make contacts in high-resolution antibody/antigen complexes. For example, in L2 it is known that positions 50 and 53 are diverse in naturally occurring antibodies and are observed to make contact with the antigen. In contrast, the conventional approach would have been to diversify all the residues in the corresponding Complementarity Determining Region (CDR1) as defined by Kabat et al. (1991, supra), some seven residues compared to the two diversified in the library for use according to the invention. This represents a significant improvement in terms of the functional diversity required to create a range of antigen binding specificities.

In nature, antibody diversity is the result of two processes: somatic recombination of germline V, D and J gene segments to create a naive primary repertoire (so called germline and junctional diversity) and somatic hypermutation of the resulting rearranged V genes. Analysis of human antibody sequences has shown that diversity in the primary repertoire is focused at the centre of the antigen binding site whereas somatic hypermutation spreads diversity to regions at the periphery of the antigen binding site that are highly conserved in the primary repertoire (see Tomlinson et al. (1996) J. Mol. Biol., 256: 813). This complementarity has probably evolved as an efficient strategy for searching sequence space and, although apparently unique to antibodies, it can easily be applied to other polypeptide repertoires. The residues which are varied are a subset of those that form the binding site for the target. Different (including overlapping) subsets of residues in the target binding site are diversified at different stages during selection, if desired.

In the case of an antibody repertoire, an initial ‘naive’ repertoire is created where some, but not all, of the residues in the antigen binding site are diversified. As used herein in this context, the term “naive” refers to antibody molecules that have no pre-determined target. These molecules resemble those which are encoded by the immunoglobulin genes of an individual who has not undergone immune diversification, as is the case with fetal and newborn individuals, whose immune systems have not yet been challenged by a wide variety of antigenic stimuli. This repertoire is then selected against a range of antigens or epitopes. If required, further diversity can then be introduced outside the region diversified in the initial repertoire. This matured repertoire can be selected for modified function, specificity or affinity.

The invention provides two different naive repertoires of binding domains for the construction of dual specific ligands, or a naive library of dual specific ligands, in which some or all of the residues in the antigen binding site are varied. The “primary” library mimics the natural primary repertoire, with diversity restricted to residues at the centre of the antigen binding site that are diverse in the germline V gene segments (germline diversity) or diversified during the recombination process (junctional diversity). Those residues which are diversified include, but are not limited to, H50, H52, H52α, H53, H55, H56, H58, H95, H96, H97, H98, L50, L53, L91, L92, L93, L94 and L96. In the “somatic” library, diversity is restricted to residues that are diversified during the recombination process (junctional diversity) or are highly somatically mutated). Those residues which are diversified include, but are not limited to: H31, H33, H35, H95, H96, H97, H98, L30, L31, L32, L34 and L96. All the residues listed above as suitable for diversification in these libraries are known to make contacts in one or more antibody-antigen complexes. Since in both libraries, not all of the residues in the antigen binding site are varied, additional diversity is incorporated during selection by varying the remaining residues, if it is desired to do so. It shall be apparent to one skilled in the art that any subset of any of these residues (or additional residues which comprise the antigen binding site) can be used for the initial and/or subsequent diversification of the antigen binding site.

In the construction of libraries for use in the invention, diversification of chosen positions is typically achieved at the nucleic acid level, by altering the coding sequence which specifies the sequence of the polypeptide such that a number of possible amino acids (all 20 or a subset thereof) can be incorporated at that position. Using the IUPAC nomenclature, the most versatile codon is NNK, which encodes all amino acids as well as the TAG stop codon. The NNK codon is preferably used in order to introduce the required diversity. Other codons which achieve the same ends are also of use, including the NNN codon, which leads to the production of the additional stop codons TGA and TAA.

A feature of side-chain diversity in the antigen binding site of human antibodies is a pronounced bias which favours certain amino acid residues. If the amino acid composition of the ten most diverse positions in each of the V_{H}, V_{κ} and V_{λ} regions are summed, more than 76% of the side-chain diversity comes from only seven different residues, these being, serine (24%), tyrosine (14%), asparagine (11%), glycine (9%), alanine (7%), aspartate (6%) and threonine (6%). This bias towards hydrophilic residues and small residues which can provide main-chain flexibility probably reflects the evolution of surfaces which are predisposed to binding a wide range of antigens or epitopes and may help to explain the required promiscuity of antibodies in the primary repertoire.

Since it is preferable to mimic this distribution of amino acids, the distribution of amino acids at the positions to be varied preferably mimics that seen in the antigen binding site of antibodies. Such bias in the substitution of amino acids that permits selection of certain polypeptides (not just antibody polypeptides) against a range of target antigens is easily applied to any polypeptide repertoire. There are various methods for biasing the amino acid distribution at the position to be varied (including the use of tri-nucleotide mutagenesis, see WO97/08320), of which the preferred method, due to ease of synthesis, is the use of conventional degenerate codons. By comparing the amino acid profile encoded by all combinations of degenerate codons (with single, double, triple and quadruple degeneracy in equal ratios at each position) with the natural amino acid use it is possible to calculate the most representative codon. The codons (AGT)(AGC)T, (AGT)(AGC)C and (AGT)(AGC)(CT)—that is, DVT, DVC and DVY, respectively using IUPAC nomenclature—are those closest to the desired amino acid profile: they encode 22% serine and 11% tyrosine, asparagine, glycine, alanine, aspartate, threonine and cysteine. Preferably, therefore, libraries are constructed using either the DVT, DVC or DVY codon at each of the diversified positions.

### G: Antigens Capable of Increasing Ligand Half-Life

The dual specific ligands according to the invention, in one configuration thereof, are capable of binding to one or more molecules which can increase the half-life of the ligand in vivo. Typically, such molecules are polypeptides which occur naturally in vivo and which resist degradation or removal by endogenous mechanisms which remove unwanted material from the organism. For example, the molecule which increases the half-life of the organism may be selected from the following:

Proteins from the extracellular matrix; for example collagen, laminins, integrins and fibronectin. Collagens are the major proteins of the extracellular matrix. About 15 types of collagen molecules are currently known, found in different parts of the body, eg type I collagen (accounting for 90% of body collagen) found in bone, skin, tendon, ligaments, cornea, internal organs or type II collagen found in cartilage, invertebral disc, notochord, vitreous humour of the eye.
Proteins found in blood, including:

Plasma proteins such as fibrin, α-2 macroglobulin, serum albumin, fibrinogen A, fibrinogen B, serum amyloid protein A, heptaglobin, profilin, ubiquitin, uteroglobulin and β-2-microglobulin;

Enzymes and inhibitors such as plasminogen, lysozyme, cystatin C, alpha-1-antitrypsin and pancreatic trypsin inhibitor. Plasminogen is the inactive precursor of the trypsin-like serine protease plasmin. It is normally found circulating through the blood stream. When plasminogen becomes activated and is converted to plasmin, it unfolds a potent enzymatic domain that dissolves the fibrinogen fibers that entgangle the blood cells in a blood clot. This is called fibrinolysis.
Immune system proteins, such as IgE, IgG, IgM.
Transport proteins such as retinol binding protein, α-1 microglobulin.
Defensins such as beta-defensin 1, Neutrophil defensins 1,2 and 3.
Proteins found at the blood brain barrier or in neural tissues, such as melanocortin receptor, myelin, ascorbate transporter.
Transferrin receptor specific ligand-neuropharmaceutical agent fusion proteins (see U.S. Pat. No. 5,977,307); brain capillary endothelial cell receptor, transferrin, transferrin receptor, insulin, insulin-like growth factor 1 (IGF 1) receptor, insulin-like growth factor 2 (IGF 2) receptor, insulin receptor.
Proteins localised to the kidney, such as polycystin, type IV collagen, organic anion transporter K1, Heymann's antigen.
Proteins localised to the liver, for example alcohol dehydrogenase, G250.
Blood coagulation factor X
α1 antitrypsin
HNF 1α
Proteins localised to the lung, such as secretory component (binds IgA).
Proteins localised to the Heart, for example HSP 27. This is associated with dilated cardiomyopathy.
Proteins localised to the skin, for example keratin.
Bone specific proteins, such as bone morphogenic proteins (BMPs), which are a subset of the transforming growth factor β superfamily that demonstrate osteogenic activity. Examples include BMP-2, -4, -5, -6, -7 (also referred to as osteogenic protein (OP-1) and -8 (OP-2).
Tumour specific proteins, including human trophoblast antigen, herceptin receptor, oestrogen receptor, cathepsins eg cathepsin B (found in liver and spleen).
Disease-specific proteins, such as antigens expressed only on activated T-cells: including LAG-3 (lymphocyte activation gene), osteoprotegerin ligand (OPGL) see Nature 402, 304-309; 1999, OX40 (a member of the TNF receptor family, expressed on activated T cells and the only costimulatory T cell molecule known to be specifically up-regulated in human T cell leukaemia virus type-I (HTLV-I)-producing cells.) See J Immunol. 2000 July 1; 165(1):263-70; Metalloproteases (associated with arthritis/cancers), including CG6512 Drosophila, human paraplegin, human FtsH, human AFG3L2, murine ftsH; angiogenic growth factors, including acidic fibroblast growth factor (FGF-1), basic fibroblast growth factor (FGF-2), Vascular endothelial growth factor/vascular permeability factor (VEGF/VPF), transforming growth factor-α (TGF a), tumor necrosis factor-alpha (TNF-α), angiogenin, interleukin-3 (IL-3), interleukin-8 (IL-8), platelet-derived endothelial growth factor (PD-ECGF), placental growth factor (P1GF), midkine platelet-derived growth factor-BB (PDGF), fractalkine.
Stress proteins (heat shock proteins)
HSPs are normally found intracellularly. When they are found extracellularly, it is an indicator that a cell has died and spilled out its contents. This unprogrammed cell death (necrosis) only occurs when as a result of trauma, disease or injury and therefore in vivo, extracellular HSPs trigger a response from the immune system that will fight infection and disease. A dual specific ligand which binds to extracellular HSP can be localised to a disease site.
Proteins involved in Fc transport
Brambell receptor (also known as FcRB)
This Fc receptor has two functions, both of which are potentially useful for delivery
The functions are
   (1) The transport of IgG from mother to child across the placenta
   (2) the protection of IgG from degradation thereby prolonging its serum half life of IgG. It is thought that the receptor recycles IgG from endosome. See Holliger et al, Nat Biotechnol 1997 July; 15(7):632-6.

Ligands according to the invention may designed to be specific for the above targets without requiring any increase in or increasing half life in vivo. For example, ligands according to the invention can be specific for targets selected from the foregoing which are tissue-specific, thereby enabling tissue-specific targeting of the dual specific ligand, or a dAb monomer that binds a tissue-specific therapeutically relevant target, irrespective of any increase in half-life, although this may result. Moreover, where the ligand or dAb monomer targets kidney or liver, this may redirect the ligand or dAb monomer to an alternative clearance pathway in vivo (for example, the ligand may be directed away from liver clearance to kidney clearance).

### Other Approaches to Increasing In Vivo Half-Life:

In addition to the design of dual-specific ligands in which one of the specificities is for a target protein that increases the serum half-life of the antibody polypeptide construct, antibody polypeptides as described herein can be further stabilized by linkage to a chemical moiety that increases serum half-life. In order to provide improvement in the pharmacokinetics of antibody molecules, the present invention provides single domain variable region polypeptides that are linked to polymers which provide increased stability and half-life. The attachment of polymer molecules (e.g., polyethylene glycol; PEG) to proteins is well established and has been shown to modulate the pharmacokinetic properties of the modified proteins. For example, PEG modification of proteins has been shown to alter the in vivo circulating half-life, antigenicity, solubility, and resistance to proteolysis of the protein (Abuchowski et al., J. Biol. Chem. 1977, 252:3578; Nucci et al., Adv. Drug Delivery Reviews 1991, 6:133; Francis et al., Pharmaceutical Biotechnology Vol. 3 (Borchardt, R. T. ed.); and Stability of Protein Pharmaceuticals: in vivo Pathways of Degradation and Strategies for Ptotein Stabilization 1991 pp 235-263, Plenum, N.Y.).

Both site-specific and random PEGylation of protein molecules is known in the art (See, for example, Zalipsky and Lee, Poly(ethylene glycol) Chemistry: Biotechnical and Biomedical Applications 1992, pp 347-370, Plenum, N.Y.; Goodson and Katre, 1990, Bio/Technology, 8:343; Hershfield et al., 1991, PNAS 88:7185). More specifically, random PEGylation of antibody molecules has been described at lysine residues and thiolated derivatives (Ling and Mattiasson, 1983, Immunol. Methods 59: 327; Wilkinson et al., 1987, Immunol. Letters, 15: 17; Kitamura et al., 1991, Cancer Res. 51:4310; Delgado et al., 1996 Br. J. Cancer, 73: 175; Pedley et al., 1994, Br. J. Cancer, 70:1126).

Methods of PEGylation are described herein below. Specific examples of PEGylation of antibody polypeptides, and dAbs in particular, are also provided in co-pending applications PCT/GB2004/002829, filed Jun. 30, 2004, which designated the U.S, and of U.S. provisional application No. 60/535,076, filed Jan. 8, 2004, the entirety of each of which is incorporated herein by reference.

Affinity/Activity Determination:

Isolated single domain antibody (e.g., dAb) polypeptides as described herein have affinities (dissociation constant, K_{d},=K_{off}/Kₒₙ) of at least 300 nM or less, and preferably at least 300 nM-50 pM, 200 nM-50 pM, and more preferably at least 100 nM-50 pM, 75 nM-50 pM, 50 nM-50 pM, 25 nM-50 pM, 10 nM-50 pM, 5 nM-50 pM, 1 nM-50 pM, 950 pM-50 pM, 900 pM-50 pM, 850 pM-50 pM, 800 pM-50 pM, 750 pM-50 pM, 700 pM-50 pM, 650 pM-50 pM, 600 pM-50 pM, 550 pM-50 pM, 500 pM-50 pM, 450 pM-50 pM, 400 pM-50 pM, 350 pM-50 pM, 300 pM-50 pM, 250 pM-50 pM, 200 pM-50 pM, 150 pM-50 pM, 100 pM-50 pM, 90 pM-50 pM, 80 pM-50 pM, 70 pM-50 pM, 60 pM-50 pM, or even as low as 50 pM.

The antigen-binding affinity of a variable domain polypeptide can be conveniently measured by surface plasmon resonance (SPR) using the BIAcore system (Pharmacia Biosensor, Piscataway, N.J.). In this method, antigen is coupled to the BlAcore chip at known concentrations, and variable domain polypeptides are introduced. Specific binding between the variable domain polypeptide and the immobilized antigen results in increased protein concentration on the chip matrix and a change in the SPR signal. Changes in SPR signal are recorded as resonance units (RU) and displayed with respect to time along the Y axis of a sensorgram. Baseline signal is taken with solvent alone (e.g., PBS) passing over the chip. The net difference between baseline signal and signal after completion of variable domain polypeptide injection represents the binding value of a given sample. To determine the off rate (K_{off}), on rate (Kₒₙ) and dissociation rate (K_{d}) constants, BIAcore kinetic evaluation software (e.g., version 2.1) is used.

High affinity is dependent upon the complementarity between a surface of the antigen and the CDRs of the antibody or antibody fragment. Complementarity is determined by the type and strength of the molecular interactions possible between portions of the target and the CDR, for example, the potential ionic interactions, van der Waals attractions, hydrogen bonding or other interactions that can occur. CDR3 tends to contribute more to antigen binding interactions than CDRs 1 and 2, probably due to its generally larger size, which provides more opportunity for favorable surface interactions. (See, e.g., Padlan et al., 1994, Mol. Immunol. 31: 169-217; Chothia & Lesk, 1987, J. Mol. Biol. 196: 904-917; and Chothia et al., 1985, J. Mol. Biol. 186: 651-663.) High affinity indicates single immunoglobulin variable domain/antigen pairings that have a high degree of complementarity, which is directly related to the structures of the variable domain and the target.

The structures conferring high affinity of a single immunoglobulin variable domain polypeptide for a given antigen can be highlighted using molecular modeling software that permits the docking of an antigen with the polypeptide structure. Generally, a computer model of the structure of a single immunoglobulin variable domain of known affinity can be docked with a computer model of a polypeptide or other target antigen of known structure to determine the interaction surfaces. Given the structure of the interaction surfaces for such a known interaction, one can then predict the impact, positive or negative, of conservative or less-conservative substitutions in the variable domain sequence on the strength of the interaction, thereby permitting the rational design of improved binding molecules.

Multimeric Forms of Antibody Single Variable Domains:

In one aspect, an antibody polypeptide construct (e.g., a dAb) as described herein is multimerized, as for example, hetero- or homodimers, hetero- or homotrimers, hetero- or homotetramers, or higher order hetero- or homomultimers (e.g., hetero- or homo-pentamer and up to octomers). Multimerization can increase the strength of antigen binding through the avidity effect, wherein the strength of binding is related to the sum of the binding affinities of the multiple binding sites.

Hetero- and Homomultimers are prepared through expression of single domain antibodies fused, for example, through a peptide linker, leading to the configuration dAb-linker-dAb or a higher multiple of that arrangement. The multimers can also be linked to additional moieties, e.g., a polypeptide sequence that increases serum half-life or another effector moiety, e.g., a toxin or targeting moiety; e.g., PEG. Any linker peptide sequence can be used to generate hetero- or homomultimers, e.g., a linker sequence as would be used in the art to generate an scFv. One commonly useful linker comprises repeats of the peptide sequence (Gly₄Ser)ₙ(SEQ ID NO: 7), wherein n=1 to about 10 (e.g., n=1, 2, 3, 4, 5, 6, 7, 8, 9, or 10). For example, the linker can be (Gly₄Ser)₃ (SEQ ID NO: 8), (Gly₄Ser)₅ (SEQ ID NO: 9), (Gly₄Ser)₇ (SEQ ID NO: 10) or another multiple of the (Gly₄Ser) (SEQ ID NO: 7) sequence.

An alternative to the expression of multimers as monomers linked by peptide sequences is linkage of the monomeric immunoglobulin variable domains post-translationally through, for example, disulfide bonding or other chemical linkage. For example, a free cysteine is engineered, e.g., at the C-terminus of the monomeric polypeptide, permits disulfide bonding between monomers. In this aspect or others requiring a free cysteine, the cysteine is introduced by including a cysteine codon (TGT, TGC) into a PCR primer adjacent to the last codon of the dAb sequence (for a C-terminal cysteine, the sequence in the primer will actually be the reverse complement, i.e., ACA or GCA, because it will be incorporated into the downstream PCR primer) and immediately before one or more stop codons. If desired, a linker peptide sequence, e.g., (Gly₄Ser)ₙ (SEQ ID NO: 7) is placed between the dAb sequence and the free cysteine. Expression of the monomers having a free cysteine residue results in a mixture of monomeric and dimeric forms in approximately a 1:1 mixture. Dimers are separated from monomers using gel chromatography, e.g., ion-exchange chromatography with salt gradient elution.

Alternatively, an engineered free cysteine is used to couple monomers through thiol linkages to a multivalent chemical linker, such as a trimeric maleimide molecule (e.g., Tris[2-maleimidoethyljamine, TMEA) or a bi-maleimide PEG molecule (available from, for example, Nektar (Shearwater).

In one embodiment, a homodimer or heterodimer of the invention includes V_{H} or V_{L} domains which are covalently attached at a C-terminal amino acid to an immunoglobulin C_{H}1 domain or C_{κ} domain, respectively. Thus the hetero- or homodimer may be a Fab-like molecule wherein the antigen binding domain contains associated V_{H} and/or V_{L} domains covalently linked at their C-termini to a C_{H1} and C_{κ} domain respectively. In addition, or alternatively, a dAb multimer of the invention may be modeled on the camelid species which express a large proportion of fully functional, highly specific antibodies that are devoid of light chain sequences. The camelid heavy chain antibodies are found as homodimers of a single heavy chain, dimerized via their constant regions. The variable domains of these camelid heavy chain antibodies are referred to as V_{H}H domains and retain the ability, when isolated as fragments of the V_{H} chain, to bind antigen with high specificity ((Hamers-Casterman et al., 1993, Nature 363: 446-448; Gahroudi et al., 1997, FEBS Lett. 414: 521-526). Thus, an antibody single variable domain multimer of the invention may be constructed, using methods known in the art, and described above, to possess the V_{H}H conformation of the camelid species heavy chain antibodies.

### PEGylation of Antibody Polypeptides

The present invention provides PEGylated antibody polypeptide (e.g., dAb) monomers and multimers which provide increased half-life and resistance to degredation without a loss in activity (e.g., binding affinity) relative to non-PEGylated antibody polypeptides.

Antibody polypeptide molecules as described herein can be coupled, using methods known in the art, to polymer molecules (preferably PEG) useful for achieving the increased half-life and degradation resistance properties. Polymer moieties which can be utilized in the invention can be synthetic or naturally occurring and include, but are not limited to straight or branched chain polyalkylene, polyalkenylene or polyoxyalkylene polymers, or a branched or unbranched polysaccharide such as a homo- or heteropolysaccharide. Preferred examples of synthetic polymers which can be used in the invention include straight or branched chain poly(ethylene glycol) (PEG), poly(propylene glycol), or poly(vinyl alcohol) and derivatives or substituted forms thereof. Particularly preferred substituted polymers for linkage to antibody polypeptides as described herein include substituted PEG, including methoxy(polyethylene glycol). Naturally occurring polymer moieties which can be used in addition to or in place of PEG include lactose, amylose, dextran, or glycogen, as well as derivatives thereof which would be recognized by one of skill in the art. Derivatized forms of polymer molecules include, for example, derivatives which have additional moieties or reactive groups present therein to permit interaction with amino acid residues of the antibody polypeptides described herein. Such derivatives include N-hydroxylsuccinimide (NHS) active esters, succinimidyl propionate polymers, and sulfhydryl-selective reactive agents such as maleimide, vinyl sulfone, and thiol. Particularly preferred derivatized polymers include, but are not limited to PEG polymers having the formulae: PEG-O—CH₂CH₂CH₂—CO₂—NHS; PEG-O—CH₂—NHS; PEG-O—CH₂CH₂—CO₂—NHS; PEG-S—CH₂CH₂—CO—NHS; PEG-O₂CNH—CH(R)—CO₂—NHS; PEG-NHCO—CH₂CH₂—CO—NHS; and PEG-O—CH₂—CO₂—NHS; where R is (CH₂)₄)NHCO₂(mPEG). PEG polymers can be linear molecules, or can be branched wherein multiple PEG moieties are present in a single polymer. Some particularly preferred PEG derivatives which are useful in the invention include, but are not limited to the following:

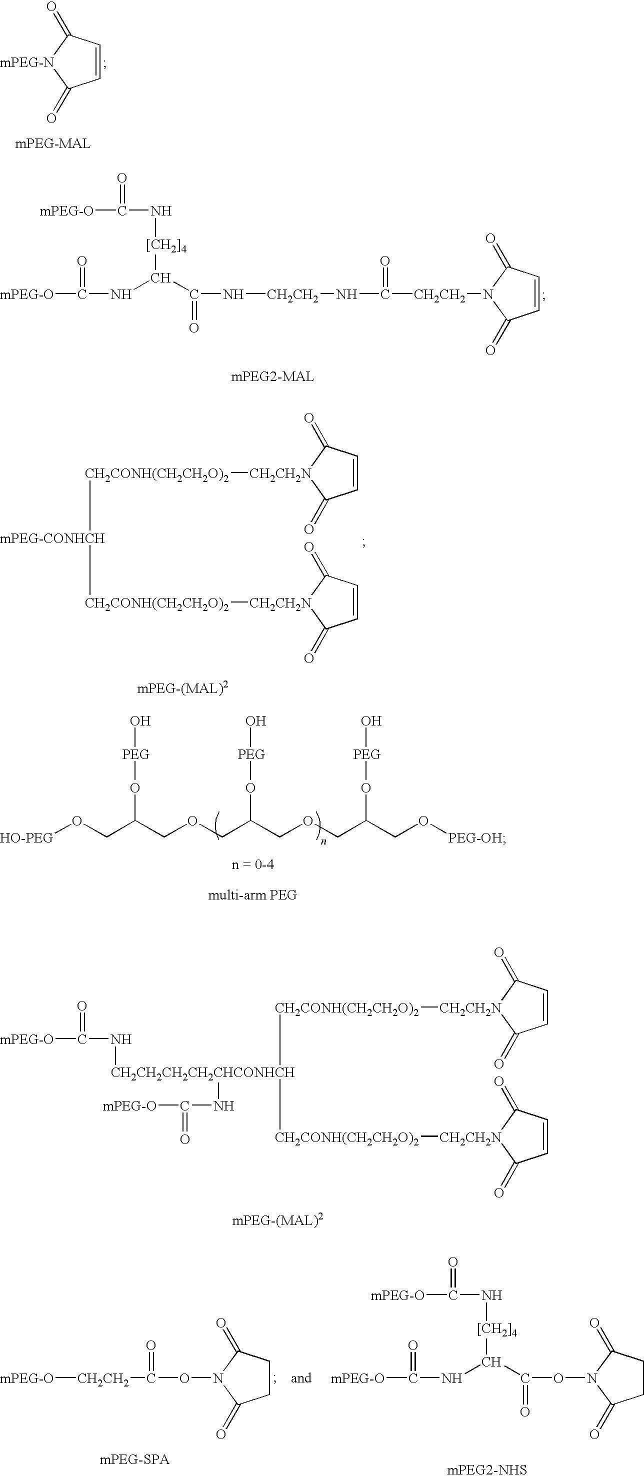

The reactive group (e.g., MAL, NHS, SPA, VS, or Thiol) may be attached directly to the PEG polymer or may be attached to PEG via a linker molecule.

The size of polymers useful in the invention can be in the range of between 500 Da to 60 kDa, for example, between 1000 Da and 60 kDa, 10 kDa and 60 kDa, 20 kDa and 60 kDa, 30 kDa and 60 kDa, 40 kDa and 60 kDa, and up to between 50 kDa and 60 kDa. The polymers used in the invention, particularly PEG, can be straight chain polymers or may possess a branched conformation. Depending on the combination of molecular weight and conformation, the polymer molecules, when attached to an antibody construct (e.g., dAb) monomer or multimer, will yield a molecule having an average hydrodynamic size of between 24 and 500 kDa. The hydrodynamic size of a polymer molecule used herein refers to the apparent size of a molecule (e.g., a protein molecule) based on the diffusion of the molecule through an aqueous solution. The diffusion, or motion of a protein through solution can be processed to derive an apparent size of the protein, where the size is given by the Stokes radius or hydrodynamic radius of the protein particle. The “hydrodynamic size” of a protein depends on both mass and shape (conformation), such that two proteins having the same molecular mass may have differing hydrodynamic sizes based on the overall conformation of the protein. The hydrodynamic size of a PEG-linked antibody single variable domain (including single domain antibody multimers as described herein) can be in the range of 24 kDa to 500 kDa; 30 to 500 kDa; 40 to 500 kDa; 50 to 500 kDa; 100 to 500 kDa; 150 to 500 kDa; 200 to 500 kDa; 250 to 500 kDa; 300 to 500 kDa; 350 to 500 kDa; 400 to 500 kDa and 450 to 500 kDa. Preferably the hydrodynamic size of a PEGylated dAb is 30 to 40 kDa; 70 to 80 kDa or 200 to 300 kDa. The size of a polymer molecule attached to an antibody polypeptide, such as a dAb or dAb multimer, can be thus varied depending on the desired application. For example, where the PEGylated dAb is intended to leave the circulation and enter into peripheral tissues, it is desirable to keep the size of the attached polymer low to facilitate extravazation from the blood stream. Alternatively, where it is desired to have the PEGylated dAb remain in the circulation for a longer period of time, a higher molecular weight polymer can be used (e.g., a 30 to 60 kDa polymer).

The polymer (PEG) molecules useful in the invention can be attached to antibody polypeptide constructs using methods which are well known in the art. The first step in the attachment of PEG or other polymer moieties to an antibody polypeptide monomer or multimer of the invention is the substitution of the hydroxyl end-groups of the PEG polymer by electrophile-containing functional groups. Particularly, PEG polymers are attached to either cysteine or lysine residues present in the antibody polypeptide monomers or multimers. The cysteine and lysine residues can be naturally occurring, or can be engineered into the antibody polypeptide molecule. For example, cysteine residues can be recombinantly engineered at the C-terminus of a dAb polypeptide, or residues at specific solvent accessible locations in a dAb or other antibody polypeptide can be substituted with cysteine or lysine. In a preferred embodiment, a PEG moiety is attached to a cysteine residue which is present in the hinge region at the C-terminus of a dAb monomer or multimer as described herein.

In one embodiment, the PEG polymer(s) is attached to one or more cysteine or lysine residues present in a framework region (FWs) and one or more heterologous CDRs of a dAb. CDRs and framework regions are those regions of an immunoglobulin variable domain as defined in the Kabat database of Sequences of Proteins of Immunological Interest (Kabat et al. (1991) Sequences of proteins of immunological interest, U.S. Department of Health and Human Services). In a preferred embodiment, a PEG polymer is linked to a cystine or lysine residue in the V_{H} framework segment DP47, or the Vₖ framework segment DPK9. Cysteine and/or lysine residues of DP47 which can be linked to PEG include the cysteine at positions 22, or 96 and the lysine at positions 43, 65, 76, or 98 of SEQ ID NO: 1 (FIG. 32). Cysteine and/or lysine residues of DPK9 which can be linked to PEG according to the invention include the cysteine residues at positions 23, or 88 and the lysine residues at positions 39, 42, 45, 103, or 107 of SEQ ID NO: 2 (FIG. 33). In addition, specific cysteine or lysine residues can be linked to PEG in the V_{H} canonical framework region DP38, or DP45.

In addition, specific solvent accessible sites in a dAb molecule which are not naturally occurring cysteine or lysine residues can be mutated to a cysteine or lysine for attachment of a PEG polymer. Solvent accessible residues in any given dAb monomer or multimer can be determined using methods known in the art such as analysis of the crystal structure of a given dAb. For example, using the solved crystal structure of the V_{H} dAb HEL4 (which binds hen egg lysozyme; see below),

Primary amino acid sequence of HEL4.

(SEQ ID NO: 5)

| | |
|---|---|
| 1 | EVQLLESGGG LVQPGGSLRL SCAASGFRIS DEDMGWVRQA |
| | PGKGLEWVSS |
| 51 | IYGPSGSTYY ADSVKGRFTI SRDNSKNTLY LQMNSLRAED |
| | TAVYYCASAL |
| 101 | EPLSEPLGFW GQGTLVTVSS |

Primary amino acid sequence of Vₖ dummy.

(SEQ ID NO: 6)

| | |
|---|---|
| 1 | DIQMTQSPSS LSASVGDRVT ITCRASQSIS SYLNWYQQKP |
| | GKAPKLLIYA |
| 51 | ASSLQSGVPS RFSGSGSGTD FTLTISSLQP EDFATYYCQQ |
| | SYSTPNTFGQ |
| 101 | GTKVEIKR |

the residues Gln-12, Pro-41, Asp-62, Glu-89, Gln-112, Leu-115, Thr-117, Ser-119, and Ser-120 have been identified as being solvent accessible, and would be attractive candidates for mutation to cysteine or lysine residues for the attachment of a PEG polymer. In addition, using the solved crystal structure of the V_{κ} dummy dAb (see above), the residues Val-15, Pro-40, Gly-41, Ser-56, Gly-57, Ser-60, Pro-80, Gly-71, Gln-100, Lys-107, and Arg-108 have been identified as being solvent accessible, and would be attractive candidates for mutation to cysteine or lysine residues for the attachment of a PEG polymer. In one embodiment, a PEG polymer is linked to multiple solvent accessible cysteine or lysine residues, or to solvent accessible residues which have been mutated to a cysteine or lysine residue. Alternatively, only one solvent accessible residue is linked to PEG, either where the particular antibody polypeptide construct only possesses one solvent accessible cysteine or lysine (or residue modified to a cysteine or lysine) or where a particular solvent accessible residue is selected from among several such residues for PEGylation.

Several attachment schemes which are useful in the invention are provided by the company Nektar (SanCarlos, Calif.). For example, where attachment of PEG or other polymer to a lysine residue is desired, active esters of PEG polymers which have been derivatized with N-hydroxylsuccinimide, such as succinimidyl propionate may be used. Where attachment to a cysteine residue is intended, PEG polymers which have been derivatized with sulfhydryl-selective reagents such as maleimide, vinyl sulfone, or thiols may be used. Other examples of specific embodiments of PEG derivatives which may be used according to the invention to generate PEGylated dAbs may be found in the Nektar Catalog (available on the world wide web at nektar.com). In addition, several derivitized forms of PEG may be used according to the invention to facilitate attachment of the PEG polymer to a dAb monomer or multimer of the invention. PEG derivatives useful in the invention include, but are not limited to PEG-succinimidyl succinate, urethane linked PEG, PEG phenylcarbonate, PEG succinimidyl carbonate, PEG-carboxymethyl azide, dimethylmaleic anhydride PEG, PEG dithiocarbonate derivatives, PEG-tresylates (2,2,2-trifluoroethanesolfonates), mPEG imidoesters, and other as described in Zalipsky and Lee, (1992) (“Use of functionalized poly(ethylene glycol)s for modification of peptides” in Poly(Ethylene Glycol) Chemistry: Biotechnical and Biomedical Applications, J. Milton Harris, Ed., Plenum Press, N.Y.).

In each of the above embodiments, the PEG polymers can be attached to any amenable residue present in the antibody polypeptide construct peptides, or, preferably, one or more residues of the construct can be modified or mutated to a cysteine or lysine residue which may then be used as an attachment point for a PEG polymer. Preferably, a residue to be modified in this manner is a solvent accessible residue; that is, a residue, which when the antibody polypeptide construct is in its natural folded configuration is accessible to an aqueous environment and to a derivatized PEG polymer. Once one or more of these residues is mutated to a cysteine residue according to the invention, it is available for PEG attachment using a linear or branched MAL derivatized PEG (MAL-PEG).

In one embodiment, there is provided an antibody construct comprising an antibody single variable domain and PEG polymer wherein the ratio of PEG polymer to antibody single variable domain is a molar ratio of at least 0.25:1. In a further embodiment, the molar ratio of PEG polymer to antibody single variable domain is 0.33:1 or greater. In a still further embodiment the molar ratio of PEG polymer to antibody single variable domain is 0.5:1 or greater.

### Anti-Serum Albumin Single Variable Domains

As described above, ligands described herein comprising a single variable domain as defined herein may be selected to be specific for a target and preferably may have the added attribute of increasing the half life of a target in vivo, though not required. A dual-specific ligand may be composed of an antibody heavy chain single variable domain having a binding specificity to a first epitope or antigen, and also of an antibody light chain single variable domain having a binding specificity to a second epitope or antigen, where one or both of the antigens can be serum albumin, or one or both of the epitopes can be an epitope(s) of serum albumin. Both serum albumin epitopes can be the same, or each serum albumin epitope can be different.

In addition to these dual-specific ligands which have the attribute of increasing the half life of a target in vivo, other forms of ligands are described herein which have or consist of at least one single variable domain as defined herein which has the attribute of increasing the half life of a target in vivo, e.g., by binding serum albumin. For example, the ligand can consist of, or contain, a monomer single variable domain as defined herein which binds serum albumin; or the ligand can be in a form which comprises multiple single variable domains as defined herein, where one or more of the single variable domains binds serum albumin, i.e., a multimer. Both the multimer and the monomer can further comprise other entities in addition to the one or more single variable domain(s) which binds serum albumin, e.g., in the form of a fusion protein and/or a conjugate. Such a fusion protein preferably is a single polypeptide chain and can comprise for example two or more linked single variable domains as defined herein; the linked single variable domains can be identical to each other or they can be different from each other. Such entities include e.g., one or more additional single variable domains as defined herein, which have a specificity to an antigen or epitope other than serum albumin, and/or one or more drugs, and/or one or more target binding domains which have a specificity to an antigen or epitope other than serum albumin and which are not single variable domains as defined herein. Such a multimer may have multiple valencies with respect to its single variable domain(s), e.g., univalent, divalent, trivalent, tetravalent. Such a multimer may have the form of an IgG structure or a dual specific ligand as defined herein, as well as other structures such as IgM, IgE, IgD, or IgA, and/or fragments thereof, including but not limited to fragments such as scFv fragments, Fab, Fab′ etc. The ligand can be modified to contain additional moieties, such as a fusion protein, or a conjugate.

An antibody heavy chain single variable domain of a dual specific ligand or of a monomer ligand or of a multimer ligand as described herein, can specifically bind serum albumin and contain an amino acid sequence of an antibody heavy chain single variable domain. Such an antibody heavy chain single variable domain can be selected from, but preferably is not limited to, one of the following domains: dAb7r20, dAb7r21, dAb7r22, dAb7r23, dAb7r24, dAb7r25, dAb7r26, dAb7r27, dAb7r28, dAb7r29, dAb7r31, dAb7r32, dAb7r33, dAb7h21, dAb7h22, dAb7h23, Ab7h24, Ab7h25, Ab7h26, dAb7h27, dAb7h30 and dAb7h31, or a domain with an amino acid sequence that is at least 80% identical thereto, up to and including 85%, 90%, 95%, 96%, 97%, 98%, 99% identical thereto, and specifically binds serum albumin. Such an antibody heavy chain single variable domain can be selected from, but preferably is not limited to, a domain, preferably an antibody heavy chain single variable domain, that competes for binding to serum albumin with one of the following domains: dAb7r20, dAb7r21, dAb7r22, dAb7r23, dAb7r24, dAb7r25, dAb7r26, dAb7r27, dAb7r28, dAb7r29, dAb7r31, dAb7r32, dAb7r33, dAb7h21, dAb7h22, dAb7h23, Ab7h24, Ab7h25, Ab7h26, dAb7h27, dAb7h30 dAb7h31, dAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1, and dAb7p2, or with a domain having an amino acid sequence that is at least 80% identical thereto, up to and including 85%, 90%, 95%, 96%, 97%, 98%, 99% identical thereto, and that specifically binds serum albumin. Alternatively, the ligand can comprise, in addition to the antibody heavy chain single variable domain, an antibody light chain single variable domain which can specifically bind serum albumin and comprise an amino acid sequence of an antibody light chain single variable domain. Such an antibody light chain single variable domain can be selected from, but preferably is not limited to, one of the following domains: dAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h6, dAb7h7, dAb7h8, dAb7h9, dAb7h10, dAb7h11, dAb7h12, dAb7h13, dAb7h14, dAb7p1, and dAb7p2, or a domain with an amino acid sequence that is at least 80% identical thereto, up to and including 85%, 90%, 95%, 96%, 97%, 98%, 99% identical thereto, and that specifically binds serum albumin. Such an antibody light chain single variable domain can be an antibody light chain single variable domain, that competes for binding to serum albumin with a domain that can be selected from, but preferably not limited to, one of the following domains: dAb7r20, dAb7r21, dAb7r22, dAb7r23, dAb7r24, dAb7r25, dAb7r26, dAb7r27, dAb7r28, dAb7r29, dAb7r30, dAb7r32, dAb7r33, dAb7h21, dAb7h22, dAb7h23, Ab7h24, Ab7h25, Ab7h26, dAb7h27, dAb7h30 dAb7h31, dAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1, and dAb7p2, or a domain having an amino acid sequence that is at least 80% identical thereto, up to and including 85%, 90%, 95%, 96%, 97%, 98%, 99% identical thereto, and having binding specificity for serum albumin. In one embodiment, the ligand can be an IgG immunoglobulin having any combination of one, or two of the above dual specific ligands. In one embodiment, the ligand can contain one or more monomers of the single variable domains listed above, where if the ligand contains more than one of these single variable domains, each single variable domain can be identical to each other, or not identical to each other.

In one embodiment, the ligand can be a dual specific ligand which has a first immunoglobulin single variable domain having a first antigen or epitope binding specificity and a second immunoglobulin single variable domain having a second antigen or epitope binding specificity, the first and the second immunoglobulin single variable domains being antibody heavy chain single variable domains, and where one or both of the first and second antibody heavy chain single variable domains specifically binds to serum albumin and has an amino acid sequence of an antibody heavy chain single variable domain that can be selected from, but is preferably not limited to, one of the following antibody heavy chain single variable domains: dAb7r20, dAb7r21, dAb7r22, dAb7r23, dAb7r24, dAb7r25, dAb7r26, dAb7r27, dAb7r28, dAb7r29, dAb7r31, dAb7r32, dAb7r33, dAb7h21, dAb7h22, dAb7h23, Ab7h24, Ab7h25, Ab7h26, dAb7h27, dAb7h30 and dAb7h31, or an amino acid sequence that is at least 80% identical thereto, up to and including 85%, 90%, 95%, 96%, 97%, 98%, or 99% identical thereto. One embodiment of such a ligand is a dual specific ligand which has a first immunoglobulin single variable domain having a first antigen or epitope binding specificity and a second immunoglobulin single variable domain having a second antigen or epitope binding specificity, the first and the second immunoglobulin single variable domains being antibody heavy chain single variable domains, and where one or both of the first and second antibody heavy chain single variable domains specifically binds to serum albumin and competes for binding to serum albumin with a single variable domain which has an amino acid sequence of an antibody single variable domain that can be selected from, but is preferably not limited to, one of the following antibody single variable domains: dAb7r20, dAb7r21, dAb7r22, dAb7r23, dAb7r24, dAb7r25, dAb7r26, dAb7r27, dAb7r28, dAb7r29, dAb7r30, dAb7r31, dAb7r32, dAb7r33, dAb7h22, dAb7h23, Ab7h24, Ab7h25, Ab7h26, dAb7h27, dAb7h30 dAb7h31, dAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h6, dAb7h7, dAb7h8, dAb7h9, dAb7h10, dAb7h11, dAb7h12, dAb7h13, dAb7h14, dAb7p1, and dAb7p2, or a sequence that is at least 80% identical thereto, or up to and including 85%, 90%, 95%, 96%, 97%, 98%, 99% identical thereto. In one embodiment, the ligand can be an IgG immunoglobulin having any combination of one or two of the above dual specific ligands. In one embodiment, the ligand can contain one or more monomers of the single variable domains listed above, where if the ligand contains more than one of these single variable domains, each single variable domain can be identical to each other, or not identical to each other.

In one embodiment a dual specific ligand has a first immunoglobulin single variable domain having a first antigen or epitope binding specificity and a second immunoglobulin single variable domain having a second antigen or epitope binding specificity, the first and the second immunoglobulin single variable domains being antibody light chain single variable domains, and one or both of the first and second antibody light chain single variable domains specifically binds to serum albumin and has an amino acid sequence of an antibody light chain single variable domain that can be selected from, but is preferably not limited to, one of the following antibody light chain single variable domains dAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7ₚ1, and dAb7p2, or a sequence that is at least 80% identical thereto, or up to and including 85%, 90%, 95%, 96%, 97%, 98%, or 99% identical thereto.

In one embodiment, the ligand can be a dual specific ligand which has a first immunoglobulin single variable domain having a first antigen or epitope binding specificity and a second immunoglobulin single variable domain having a second antigen or epitope binding specificity, the first and the second immunoglobulin single variable domains being antibody light chain single variable domains, and one or both of the first and second antibody light chain single variable domains specifically binds to serum albumin and competes for binding to serum albumin with an antibody light chain single variable domain which has an amino acid sequence of an antibody single variable domain which can be selected from, but preferably is not limited to, one of the following antibody single variable domains: dAb7r20, dAb7r21, dAb7r22, dAb7r23, dAb7r24, dAb7r25, dAb7r26, dAb7r27, dAb7r28, dAb7r29, dAb7r30, dAb7r31, dAb7r32, dAb7r33, dAb7h21, dAb7h22, dAb7h23, Ab7h24, Ab7h25, Ab7h26, dAb7h27, dAb7h30 dAb7h31, dAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h6, dAb7h7, dAb7h8, dAb7h9, dAb7h10, dAb7h11, dAb7h12, dAb7h13, dAb7h14, dAb7p1 and dAb7p2, or a sequence that is at least 80% identical thereto, or up to and including 85%, 90%, 95%, 96%, 97%, 98%, or 99% identical thereto.

Described herein is a ligand which has one or more antibody heavy chain single variable domains where the one or more antibody heavy chain single variable domain specifically binds serum albumin and has an amino acid sequence of an antibody heavy chain single variable domain selected from, but preferably not limited to, that of dAb8, dAb 10, dAb36, dAb7r20, dAb7r21, dAb7r22, dAb7r23, dAb7r24, dAb7r25, dAb7r26, dAb7r27, dAb7r28, dAb7r29, dAb7r30, dAb7r31, dAb7r32, dAb7r33, dAb7h21, dAb7h22, dAb7h23, Ab7h24, Ab7h25, Ab7h26, dAb7h27, dAb7h30, dAb7h31, and a sequence that is at least 80% identical thereto, or up to and including 85%, 90%, 95%, 96%, 97%, 98%, or 99% identical thereto.

Described herein is a ligand which has one or more antibody heavy chain single variable domains, where the one or more antibody heavy chain single variable domains specifically binds serum albumin and competes for binding to serum albumin with an antibody single variable domain which has an amino acid sequence of an antibody single variable domain selected from, but preferably not limited to, that of one of the following: dAb8, dAb 10, dAb36, dAb7r20, dAb7r21, dAb7r22, dAb7r23, dAb7r24, dAb7r25, dAb7r26, dAb7r27, dAb7r28, dAb7r29, dAb7r30, dAb7r31, dAb7r32, dAb7r33, dAb7h21, dAb7h22, dAb7h23, Ab7h24, Ab7h25, Ab7h26, dAb7h27, dAb7h30, dAb7h31, dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, dAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1, and dAb7p2.

Described herein is a ligand which has an antibody heavy chain single variable domain having a binding specificity to a first antigen, or epitope thereof, and an antibody light chain single variable domain having a binding specificity to a second antigen, or epitope thereof, where one or both of the first antigen and said second antigen is serum albumin, and where the antibody heavy chain single variable domain specifically binds serum albumin and competes for binding to serum albumin with an antibody single variable domain which has an amino acid sequence of an antibody single variable domain selected from, but preferably not limited to, the group: dAb8, dAb 10, dAb36, dAb7r20, dAb7r21, dAb7r22, dAb7r23, dAb7r24, dAb7r25, dAb7r26, dAb7r27, dAb7r28, dAb7r29, dAb7r30, dAb7r31, dAb7r32, dAb7r33, dAb7h21, dAb7h22, dAb7h23, Ab7h24, Ab7h25, Ab7h26, dAb7h27, dAb7h30, dAb7h31, dAb2, dAb4, dAb7, dAb11, dAb12, dAb13, dAb15, dAb16, dAb17, dAb18, dAb 19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, dAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1, dAb7p2, and where the antibody light chain single variable domain specifically binds serum albumin and has an amino acid sequence of an antibody light chain single variable domain selected from, but preferably not limited to, that of the following: dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, drdAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1, and dAb7p2, and a sequence that is at least 80% identical thereto, or up to and including 85%, 90%, 95%, 96%, 97%, 98%, or 99% identical thereto.

Described herein is a ligand which has an antibody heavy chain single variable domain having a binding specificity to a first antigen or epitope thereof, and an antibody light chain single variable domain having a binding specificity to a second antigen or epitope thereof, wherein one or both of said first antigen and said second antigen is serum albumin, and wherein the antibody heavy chain single variable domain specifically binds serum albumin and has an amino acid sequence of an antibody heavy chain single variable domain selected from but preferably not limited to, the group: dAb8, dAb 10, dAb36, dAb7r20, dAb7r21, dAb7r22, dAb7r23, dAb7r24, dAb7r25, dAb7r26, dAb7r27, dAb7r28, dAb7r29, dAb7r30, dAb7r31, dAb7r32, dAb7r33, dAb7h21, dAb7h22, dAb7h23, Ab7h24, Ab7h25, Ab7h26, dAb7h27, dAb7h30, dAb7h31, and a sequence that is at least 80% identical thereto, or up to and including 85%, 90%, 95%, 96%, 97%, 98%, or 99% identical thereto, and where the antibody light chain single variable domain specifically binds serum albumin and competes for binding to serum albumin with an antibody single variable domain which comprises an amino acid sequence of an antibody single variable domain selected from, but preferably not limited to the group: dAb8, dAb10, dAb36, dAb7r20, dAb7r21, dAb7r22, dAb7r23, dAb7r24, dAb7r25, dAb7r26, dAb7r27, dAb7r28, dAb7r29, dAb7r30, dAb7r31, dAb7r32, dAb7r33, dAb7h21, dAb7h22, dAb7h23, Ab7h24, Ab7h25, Ab7h26, dAb7h27, dAb7h30, dAb7h31, dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, dAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1,dAb7h2, dAb7h6, dAb7h7, dAb7h8, dAb7h9, dAb7h10, dAb7h11, dAb7h12, dAb7h13, dAb7h14, dAb7p1 and dAb7p2.

Described herein is a ligand which has one or more antibody heavy chain single variable domains having a binding specificity to a first antigen or epitope thereof, and one or more antibody light chain single variable domains having a binding specificity to a second antigen or epitope thereof, wherein one or both of the first antigen and the second antigen is serum albumin, and wherein the one or more antibody heavy chain single variable domains specifically binds serum albumin and competes for binding to serum albumin with an antibody single variable domain which has an amino acid sequence of an antibody single variable domain selected from, but preferably not limited to, the group: dAb8, dAb 10, dAb36, dAb7r20, dAb7r21, dAb7r22, dAb7r23, dAb7r24, dAb7r25, dAb7r26, dAb7r27, dAb7r28, dAb7r29, dAb7r30, dAb7r31, dAb7r32, dAb7h33, dAb7h21, dAb7h22, dAb7h23, Ab7h24, Ab7h25, Ab7h26, dAb7h27, dAb18, dAb7h31, dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, dAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1, dAb7p2, and where the one or more antibody light chain single variable domains specifically binds serum albumin and comprises an amino acid sequence of an antibody light chain single variable domain selected from, but preferably not limited to, the group: dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, drdAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1, and dAb7p2, and an amino acid sequence that is at least 80% identical thereto.

Described herein is a ligand which has one or more antibody heavy chain single variable domains having a binding specificity to a first antigen or epitope thereof, and one or more antibody light chain single variable domains having a binding specificity to a second antigen or epitope thereof, where one or both of said first antigen and said second antigen is serum albumin, and where the one or more antibody heavy chain single variable domains specifically binds serum albumin and has an amino acid sequence of an antibody heavy chain single variable domain selected from, but preferably not limited to, the group: dAb8, dAb 10, dAb36, dAb7r20, dAb7r21, dAb7r22, dAb7r23, dAb7r24, dAb7r25, dAb7r26, dAb7r27, dAb7r28, dAb7r29, dAb7r30, dAb7r31, dAb7h32, dAb7r33, dAb7h21, dAb7h22, dAb7h23, Ab7h24, Ab7h25, Ab7h26, dAb7h27, dAb7h30, dAb7h31, and a sequence that is at least 80% identical thereto, or up to and including 85%, 90%, 95%, 96%, 97%, 98%, or 99% identical thereto, and where the one or more antibody light chain single variable domains specifically binds serum albumin and competes for binding to serum albumin with an antibody single variable domain which has an amino acid sequence of an antibody single variable domain selected from the group: dAb8, dAb 10, dAb36, dAb7r20, dAb7r21, dAb7r22, dAb7r23, dAb7r24, dAb7r25, dAb7r26, dAb7r27, dAb7r28, dAb7r29, dAb7r30, dAb7r31, dAb7h32, dAb7r33, dAb7h21, dAb7h22, dAb7h23, Ab7h24, Ab7h25, Ab7h26, dAb7h27, dAb7h30, dAb7h31, dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, dAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1 and dAb7p2.

Described herein is a ligand which has one or more antibody light chain single variable domains and where the one or more antibody light chain single variable domains specifically binds serum albumin and has an amino acid sequence of an antibody light chain single variable domain selected from, but preferably not limited to, the group: dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, drdAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1, dAb7p2, and a sequence that is at least 80% identical thereto, or up to and including 85%, 90%, 95%, 96%, 97%, 98%, or 99% identical thereto.

Described herein is a ligand which has one or more antibody light chain single variable domains, where the one or more antibody light chain single variable domains specifically binds serum albumin and competes for binding to serum albumin with an antibody single variable domain which has an amino acid sequence of an antibody single variable domain selected from, but preferably not limited to, the group: dAb8, dAb 10, dAb36, dAb7r20, dAb7r21, dAb7r22, dAb7r23, dAb7r24, dAb7r25, dAb7r26, dAb7r27, dAb7r28, dAb7r29, dAb7r30, dAb7r31, dAb7r32, dAb7r33, dAb7h21, dAb7h22, dAb7h23, Ab7h24, Ab7h25, Ab7h26, dAb7h27, dAb7h30, dAb7h31, dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, dAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1, and dAb7p2.

Described herein is a ligand which has one or more single variable domains, where the one or more single variable domains specifically binds serum albumin and competes for binding to serum albumin with an antibody single variable domain which has an amino acid sequence of an antibody single variable domain selected from, but preferably not limited to the group: dAb8, dAb 10, dAb36, dAb7r20, dAb7r21, dAb7r22, dAb7r23, dAb7r24, dAb7r25, dAb7r26, dAb7r27, dAb7r28, dAb7r29, dAb7h30, dAb7r31, dAb7r32, dAb7r33, dAb7h21, dAb7h22, dAb7h23, Ab7h24, Ab7h25, Ab7h26, dAb7h27, dAb7h30, dAb7h31, dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, dAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1, and dAb7p2. Preferably, the one or more single variable domains comprises a scaffold selected from, but preferably not limited to, the group consisting of CTLA-4, lipocallin, SpA, an Affibody, an avimer, GroE1 and fibronectin, and competes for binding to serum albumin with an antibody single variable domain which has an amino acid sequence of an antibody single variable domain selected from, but preferably not limited to the group: dAb8, dAb 10, dAb36, dAb7r20, dAb7r21, dAb7r22, dAb7r23, dAb7r24, dAb7r25, dAb7r26, dAb7r27, dAb7r28, dAb7h29, dAb7r30, dAb7r31, dAb7r32, dAb7r33, dAb7h21, dAb7h22, dAb7h23, dAb7h24, Ab7h25, Ab7h26, dAb7h27, dAb7h30, dAb7h31, dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, dAb7m12, dAb7m16, dAb7m26, dAb7r1, dAb7r3, dAb7r4, dAb7r5, dAb7r7, dAb7r8, dAb7r13, dAb7r14, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1, and dAb7p2.

Described herein is a ligand which has a first immunoglobulin single variable domain having a first antigen or epitope binding specificity and a second immunoglobulin single variable domain having a second antigen or epitope binding specificity, where the first and the second immunoglobulin single variable domains are antibody heavy chain single variable domains, where the first antibody heavy chain single variable domains specifically binds to serum albumin and has an amino acid sequence of an antibody heavy chain single variable domain selected from, but preferably not limited to, the group: dAb8, dAb 10, dAb36, dAb7r20, dAb7r21, dAb7r22, dAb7r23, dAb7r24, dAb7r25, dAb7r26, dAb7r27, dAb7r28, dAb7r29, dAb7r30, dAb7r31, dAb7r32, dAb7h33, dAb7h21, dAb7h22, dAb7h23, Ab7h24, Ab7h25, Ab7h26, dAb7h27, dAb7h30, dAb7h31, and a sequence that is at least 80% identical thereto, or up to and including 85%, 90%, 95%, 96%, 97%, 98%, or 99% identical thereto, and where the second antibody heavy chain single variable domains specifically binds to serum albumin and competes for binding to serum albumin with an antibody single variable domain which has an amino acid sequence of an antibody single variable domain selected from the group: dAb8, dAb 10, dAb36, dAb7r20, dAb7r21, dAb7r22, dAb7r23, dAb7r24, dAb7r25, dAb7r26, dAb7r27, dAb7r28, dAb7r29, dAb7r30, dAb7r31, dAb7h32, dAb7r33, dAb7h21, dAb7h22, dAb7h23, Ab7h24, Ab7h25, Ab7h26, dAb7h27, dAb7h30, dAb7h31, dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, dAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1, and dAb7p2.

Described herein is a ligand which has a first immunoglobulin single variable domain having a first antigen or epitope binding specificity and a second immunoglobulin single variable domain having a second antigen or epitope binding specificity, where the first and the second immunoglobulin single variable domains are antibody light chain single variable domains, where the first antibody light chain single variable domain specifically binds to serum albumin and has an amino acid sequence of an antibody light chain single variable domain selected from, but preferably not limited to, the group: dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, drdAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1, dAb7p2, and a sequence that is at least 80% identical thereto, or up to and including 85%, 90%, 95%, 96%, 97%, 98%, or 99% identical thereto, and where the second antibody light chain single variable domain specifically binds to serum albumin and competes for binding to serum albumin with an antibody single variable domain which has an amino acid sequence of an antibody single variable domain selected from, but preferably not limited to, the group: dAb8, dAb 10, dAb36, dAb7r20, dAb7r21, dAb7r22, dAb7r23, dAb7r24, dAb7r25, dAb7r26, dAb7r27, dAb7r28, dAb7r29, dAb7r30, dAb7r31, dAb7r32, dAb7r33, dAb7h21, dAb7h22, dAb7h23, dAb7h24, dAb7h25, Ab7h26, dAb7h27, dAb7h30, dAb7h31, dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, dAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1, and dAb7p2.

Embodiments of ligands described supra and herein, also includes those having a structure comprising an IgG immunoglobulin having any combination of one, or two of the above dual specific ligands, and/or single variable domains comprising non-immunoglobulin scaffolds. Such an immunoglobulin structure can have various combinations of antibody single variable domains, including an IgG structure that contains four antibody heavy chain single variable domains, or an IgG structure that contains four antibody light chain single variable domains, as well as an IgG structure that contains two pairs of chains, each pair containing an antibody heavy chain single variable domain and an antibody light chain single variable domain. In addition to these IgG structures, the ligands described herein can contain one or more monomers of a single variable domain, including but preferably not limited to the single variable domains listed above, where if the ligand contains more than one of these single variable domains, the single variable domains can be identical to each other, or not identical to each other.

Embodiments of ligands comprising one or more single variable domains include, but preferably are not limited to, the dAbs described herein, dual specific monomers comprising at least one single variable domain, dual specific IgG molecules containing antibody single chain monomers, and multivalent IgG molecules comprising antibody single chain monomers as described herein. These embodiments, can further comprise a binding site for a generic ligand. The generic ligand can include, but preferably is not limited to, protein A, protein L and protein G. For such dual specific ligands, including those in an IgG format, the target(s) for each second antigen or epitope binding specificity includes, but preferably is not limited to, a binding specificity for an antigen which can be characterized in a group selected from cytokines, cytokine receptors, enzymes, enzyme co-factors and DNA binding proteins, and can be selected from, but preferably is not limited to, EPO receptor, ApoE, Apo-SAA, BDNF, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-β1, insulin, IFN-γ IL-1β, IL-2, IL-3, IL-4, IL-5, IL-6, IL-7, 1L-8 (72 a.a.), IL-8 (77 a.a), IL-9, IL-10, IL-11, IL-12, IL-13, IL-15, IL-16, IL-17, IL-18 (IGIF),Inhibin α, Inhibin β, IP-10 keratinocyte growth factor-2 (KGF-2), KGF, Leptin, L1F, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a), MIG, MLP-1α, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF 1α, SDF1β TGF-β, TGF-β2, TGF-β, TNF-β, TNF receptor 1, TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER3, HER4, CD4, human chemokine receptors CXCR4 or CCR5, non-structural protein type 3 (NS3) from the hepatitis C virus, TNF-alpha, IgE, IFN-gamma, MMP-12, CEA, H. pylori, TB, influenza, Hepatitis E, MMP-12, internalising receptors such as the epidermal growth factor receptor (EGFR), ErBb2 receptor on tumor cells, an internalising cellular receptor, LDL receptor, FGF2 receptor, ErbB2 receptor, transferrin receptor, PDGF receptor, VEGF receptor, PsmAr, an extracellular matrix protein, elastin, fibronectin, laminin, α1-antitrypsin, tissue factor protease inhibitor, PDK1, GSK1, Bad, caspase-9, Forkhead, an antigen of Helicobacter pylori, an antigen of Mycobacterium tuberculosis, and an antigen of influenza virus. In such a dual-specific ligand, including those dual specific ligands present in an IgG format, one or both single variable domains specifically binds an epitope or antigen with a dissociation constant (Kd) that can be selected from, but is preferably not limited to, 1×10⁻³ M or less, 1×10⁻⁴ M or less, 1×10⁻⁵ M or less, 1×10⁻⁶ M or less, 1×10⁻⁻⁷ M or less, 1×10⁻⁸ M or less, and 1×10⁻⁹ M or less, as determined, for example, by surface plasmon resonance. Such a dual-specific ligand, including those dual specific ligands present in an IgG format, can further contain one or more entities including, but preferably is not limited to a label, a tag and a drug. Such a dual-specific ligand, including those dual specific ligands present in an IgG format, as well as a multimeric ligand that contains one or more monomers of the single variable domains listed above, can be present in a kit, and in a composition, including a pharmaceutical composition, containing the dual specific ligand and a carrier thereof.

Similarly, for a ligand comprising one or more single variable domains as described herein, including a ligand in monomeric form and a ligand in multimeric form as defined supra, the one or more single variable domains specifically binds an epitope or antigen with a dissociation constant (Kd) that can be selected from, but is preferably not limited to, 1×10⁻³ M or less, 1×10⁻⁴ M or less, 1×10⁻⁵ M or less, 1×10⁻⁶ M or less, 1×10⁻⁷ M or less, 1×10⁻⁸ M or less, and 1×10⁻⁹ M or less, as determined, for example, by surface plasmon resonance. Such a ligand can further contain one or more entities including, but preferably not limited to a label, a tag and a drug. Such ligand can be present in a kit, a composition, including a pharmaceutical composition, containing the ligand and a carrier thereof.

Percent identity, where recited herein can refer to the percent identity along the entire stretch of the length of the amino acid or nucleotide sequence. When specified, the percent identity of the amino acid or nucleic acid sequence refers to the percent identity to sequence(s) from one or more discrete regions of the referenced amino acid or nucleic acid sequence, for instance, along one or more antibody CDR regions, and/or along one or more antibody variable domain framework regions. For example, the sequence identity at the amino acid level across one or more CDRs of a polypeptide can have at least 80%, 85%, 90%, 91%, 92%, 93%, 94%, 95%, 96%, 97%, 98%, or 99% or identity to the amino acid sequence of corresponding CDRs of an antibody heavy or light chain single variable domain. Similarly, the sequence identity at the amino acid level across one or more framework regions of a polypeptide can have at least 80%, 85%, 90%, 91%, 92%, 93%, 94%, 95%, 96%, 97%, 98%, or 99% or higher identity to the amino acid sequence of a corresponding framework of an antibody heavy or light chain single variable domain. At the nucleic acid level, the nucleic acid sequence encoding one or more CDRs of a polypeptide can have at least 70%, 75%, 80%, 85%, 90%, 91%, 92%, 93%, 94%, 95%, 96%, 97%, 98%, or 99% or higher identity to the nucleic acid sequence encoding corresponding CDRs of an antibody heavy or light chain single variable domain. At the nucleic acid level, the nucleic acid sequence encoding one or more framework regions of a polypeptide can have at least 70%, 75%, 80%, 85%, 90%, 91%, 92%, 93%, 94%, 95%, 96%, 97%, 98%, or 99% or higher identity to the nucleic acid sequence encoding corresponding framework regions of an antibody heavy or light chain single variable domain, respectively. The framework regions (FW) are preferably from an antibody framework region, such as the human V3-23/DP47/JH4B heavy or the human kappa light chain DPK9JK1. If the framework region(s) is that found in the human V3-23/DP47/JH4B heavy chain V region, the percent identity can be targeted to its framework regions and/or preferably to one or more of the CDR regions as illustrated in FIG. 45. If the framework is that found in the human DPK9JK1 light chain V region, the percent identity can be compared to its referenced framework regions and/or preferably to one or more of the CDR regions as illustrated in FIG. 45.

The CDRs are preferably those of an antibody variable domain, preferably, but not limited to those of antibody single variable domains.

In some embodiments, the structural characteristic of percent identity is coupled to a functional aspect. For instance, in some embodiments, a nucleic acid sequence or amino acid sequence with less than 100% identity to a referenced nucleic acid or amino acid sequence is also required to display at least one functional aspect of the reference amino acid sequence or of the amino acid sequence encoded by the referenced nucleic acid. In other embodiments, a nucleic acid sequence or amino acid sequence with less than 100% identity to a referenced nucleic acid or amino acid sequence, respectively, is also required to display at least one functional aspect of the reference amino acid sequence or of the amino acid sequence encoded by the referenced nucleic acid, but that functional characteristic can be slightly altered, e.g., confer an increased affinity to a specified antigen relative to that of the reference.

### H: Use of f Multispecific Ligands According to the Second Configuration of the Invention

Multispecific ligands according to the method of the second configuration of the present invention may be employed in in vivo therapeutic and prophylactic applications, in vitro and in vivo diagnostic applications, in vitro assay and reagent applications, and the like. For example antibody molecules may be used in antibody based assay techniques, such as ELISA techniques, according to methods known to those skilled in the art.

As alluded to above, the multispecific ligands according to the invention are of use in diagnostic, prophylactic and therapeutic procedures. Multispecific antibodies according to the invention are of use diagnostically in Western analysis and in situ protein detection by standard immunohistochemical procedures; for use in these applications, the ligands may be labelled in accordance with techniques known to the art. In addition, such antibody polypeptides may be used preparatively in affinity chromatography procedures, when complexed to a chromatographic support, such as a resin. All such techniques are well known to one of skill in the art.

Diagnostic uses of the closed conformation multispecific ligands according to the invention include homogenous assays for analytes which exploit the ability of closed conformation multispecific ligands to bind two targets in competition, such that two targets cannot bind simultaneously (a closed conformation), or alternatively their ability to bind two targets simultaneously (an open conformation).

A true homogenous immunoassay format has been avidly sought by manufacturers of diagnostics and research assay systems used in drug discovery and development. The main diagnostics markets include human testing in hospitals, doctor's offices and clinics, commercial reference laboratories, blood banks, and the home, non-human diagnostics (for example food testing, water testing, environmental testing, bio-defence, and veterinary testing), and finally research (including drug development; basic research and academic research).

At present all these markets utilise immunoassay systems that are built around chemiluminescent, ELISA, fluorescence or in rare cases radio-immunoassay technologies. Each of these assay formats requires a separation step (separating bound from un-bound reagents). In some cases, several separation steps are required. Adding these additional steps adds reagents and automation, takes time, and affects the ultimate outcome of the assays. In human diagnostics, the separation step may be automated, which masks the problem, but does not remove it. The robotics, additional reagents, additional incubation times, and the like add considerable cost and complexity. In drug development, such as high throughput screening, where literally millions of samples are tested at once, with very low levels of test molecule, adding additional separation steps can eliminate the ability to perform a screen. However, avoiding the separation creates too much noise in the read out. Thus, there is a need for a true homogenous format that provides sensitivities at the range obtainable from present assay formats. Advantageously, an assay possesses fully quantitative read-outs with high sensitivity and a large dynamic range. Sensitivity is an important requirement, as is reducing the amount of sample required. Both of these features are features that a homogenous system offers. This is very important in point of care testing, and in drug development where samples are precious. Heterogenous systems, as currently available in the art, require large quantities of sample and expensive reagents

Applications for homogenous assays include cancer testing, where the biggest assay is that for Prostate Specific Antigen, used in screening men for prostate cancer. Other applications include fertility testing, which provides a series of tests for women attempting to conceive including beta-hcg for pregnancy. Tests for infectious diseases, including hepatitis, HIV, rubella, and other viruses and microorganisms and sexually transmitted diseases. Tests are used by blood banks, especially tests for HIV, hepatitis A, B, C, non A non B. Therapeutic drug monitoring tests include monitoring levels of prescribed drugs in patients for efficacy and to avoid toxicity, for example digoxin for arrhythmia, and phenobarbital levels in psychotic cases; theophylline for asthma. Diagnostic tests are moreover useful in abused drug testing, such as testing for cocaine, marijuana and the like. Metabolic tests are used for measuring thyroid function, anaemia and other physiological disorders and functions.

The homogenous immunoassay format is moreover useful in the manufacture of standard clinical chemistry assays. The inclusion of immunoassays and chemistry assays on the same instrument is highly advantageous in diagnostic testing. Suitable chemical assays include tests for glucose, cholesterol, potassium, and the like.

A further major application for homogenous immunoassays is drug discovery and development: high throughput screening includes testing combinatorial chemistry libraries versus targets in ultra high volume. Signal is detected, and positive groups then split into smaller groups, and eventually tested in cells and then animals. Homogenous assays may be used in all these types of test. In drug development, especially animal studies and clinical trials heavy use of immunoassays is made. Homogenous assays greatly accelerate and simplify these procedures. Other Applications include food and beverage testing: testing meat and other foods for E. coli, salmonella, etc; water testing, including testing at water plants for all types of contaminants including E. coli; and veterinary testing.

In a broad embodiment, the invention provides a binding assay comprising a detectable agent which is bound to a closed conformation multispecific ligand according to the invention, and whose detectable properties are altered by the binding of an analyte to said closed conformation multispecific ligand. Such an assay may be configured in several different ways, each exploiting the above properties of closed conformation multispecific ligands.

The assay relies on the direct or indirect displacement of an agent by the analyte, resulting in a change in the detectable properties of the agent. For example, where the agent is an enzyme which is capable of catalysing a reaction which has a detectable end-point, said enzyme can be bound by the ligand such as to obstruct its active site, thereby inactivating the enzyme. The analyte, which is also bound by the closed conformation multispecific ligand, displaces the enzyme, rendering it active through freeing of the active site. The enzyme is then able to react with a substrate, to give rise to a detectable event. In an alternative embodiment, the ligand may bind the enzyme outside of the active site, influencing the conformation of the enzyme and thus altering its activity. For example, the structure of the active site may be constrained by the binding of the ligand, or the binding of cofactors necessary for activity may be prevented.

The physical implementation of the assay may take any form known in the art. For example, the closed conformation multispecific ligand/enzyme complex may be provided on a test strip; the substrate may be provided in a different region of the test strip, and a solvent containing the analyte allowed to migrate through the ligand/enzyme complex, displacing the enzyme, and carrying it to the substrate region to produce a signal. Alternatively, the ligand/enzyme complex may be provided on a test stick or other solid phase, and dipped into an analyte/substrate solution, releasing enzyme into the solution in response to the presence of analyte.

Since each molecule of analyte potentially releases one enzyme molecule, the assay is quantitative, with the strength of the signal generated in a given time being dependent on the concentration of analyte in the solution.

Further configurations using the analyte in a closed conformation are possible. For example, the closed conformation multispecific ligand may be configured to bind an enzyme in an allosteric site, thereby activating the enzyme. In such an embodiment, the enzyme is active in the absence of analyte. Addition of the analyte displaces the enzyme and removes allosteric activation, thus inactivating the enzyme.

In the context of the above embodiments which employ enzyme activity as a measure of the analyte concentration, activation or inactivation of the enzyme refers to an increase or decrease in the activity of the enzyme, measured as the ability of the enzyme to catalyse a signal-generating reaction. For example, the enzyme may catalyse the conversion of an undetectable substrate to a detectable form thereof. For example, horseradish peroxidase is widely used in the art together with chromogenic or chemiluminescent substrates, which are available commercially. The level of increase or decrease of the activity of the enzyme may between 10% and 100%, such as 20%, 30%, 40%, 50%, 60%, 70%, 80% or 90%; in the case of an increase in activity, the increase may be more than 100%, i.e. 200%, 300%, 500% or more, or may not be measurable as a percentage if the baseline activity of the inhibited enzyme is undetectable.

In a further configuration, the closed conformation multispecific ligand may bind the substrate of an enzyme/substrate pair, rather than the enzyme. The substrate is therefore unavailable to the enzyme until released from the closed conformation multispecific ligand through binding of the analyte. The implementations for this configuration are as for the configurations which bind enzyme.

Moreover, the assay may be configured to bind a fluorescent molecule, such as a fluorescein or another fluorophore, in a conformation such that the fluorescence is quenched on binding to the ligand. In this case, binding of the analyte to the ligand will displace the fluorescent molecule, thus producing a signal. Alternatives to fluorescent molecules which are useful in the present invention include luminescent agents, such as luciferin/luciferase, and chromogenic agents, including agents commonly used in immunoassays such as HRP.

Therapeutic and prophylactic uses of multispecific ligands prepared according to the invention involve the administration of ligands according to the invention to a recipient mammal, such as a human. Multi-specificity can allow antibodies to bind to multimeric antigen with great avidity. Multispecific ligands can allow the cross-linking of two antigens, for example in recruiting cytotoxic T-cells to mediate the killing of tumour cell lines.

Substantially pure ligands or binding proteins thereof, for example dAb monomers, of at least 90 to 95% homogeneity are preferred for administration to a mammal, and 98 to 99% or more homogeneity is most preferred for pharmaceutical uses, especially when the mammal is a human. Once purified, partially or to homogeneity as desired, the ligands may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

The ligands or binding proteins thereof, for example dAb monomers, of the present invention will typically find use in preventing, suppressing or treating inflammatory states, allergic hypersensitivity, cancer, bacterial or viral infection, and autoimmune disorders (which include, but are not limited to, Type I diabetes, asthma, multiple sclerosis, rheumatoid arthritis, systemic lupus erythematosus, Crohn's disease and myasthenia gravis).

In addition to rheumatoid arthritis, anti-TNF-alpha polypeptides as described herein are applicable to the treatment of autoimmune diseases, such as (parentheticals indicate affected organ), but not limited to: Addison's disease (adrenal); autoimmune diseases of the ear (ear); autoimmune diseases of the eye (eye); autoimmune hepatitis (liver); autoimmune parotitis (parotid glands); Crohn's disease and inflammatory bowel disease (intestine); Diabetes Type I (pancreas); epididymitis (epididymis), glomerulonephritis (kidneys); Graves' disease (thyroid); Guillain-Barre syndrome (nerve cells); Hashimoto's disease (thyroid); hemolytic anemia (red blood cells); systemic lupus erythematosus (multiple tissues); male infertility (sperm); multiple sclerosis (nerve cells); myasthenia gravis (neuromuscular junction); pemphigus (primarily skin); psoriasis (skin); rheumatic fever (heart and joints); sarcoidosis (multiple tissues and organs); scleroderma (skin and connective tissues); Sjogren's syndrome (exocrine glands, and other tissues); spondyloarthropathies (axial skeleton, and other tissues); thyroiditis (thyroid); ulcerative colitis (intestine); and vasculitis (blood vessels).

In addition to rheumatoid arthritis and other chronic inflammatory disorders (e.g., Crohn's disease, psoriasis, etc.), anti-VEGF polypeptides as described herein can be used to treat diabetes, acute myeloid leukemia, leukemia and ophthalmic disorders, including macular degeneration and diabetic retinopathy.

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the antibodies or binding proteins thereof in protecting against or treating the disease are available. Methods for the testing of systemic lupus erythematosus (SLE) in susceptible mice are known in the art (Knight et al. (1978) J. Exp. Med., 147: 1653; Reinersten et al. (1978) New Eng. J. Med., 299: 515). Myasthenia Gravis (MG) is tested in SJL/J female mice by inducing the disease with soluble AchR protein from another species (Lindstrom et al. (1988) Adv. Immunol., 42: 233). Arthritis is induced in a susceptible strain of mice by injection of Type II collagen (Stuart et al. (1984) Ann. Rev. Immunol., 42: 233). A model by which adjuvant arthritis is induced in susceptible rats by injection of mycobacterial heat shock protein has been described (Van Eden et al. (1988) Nature, 331: 171). Thyroiditis is induced in mice by administration of thyroglobulin as described (Maron et al. (1980) J. Exp. Med., 152: 1115). Insulin dependent diabetes mellitus (IDDM) occurs naturally or can be induced in certain strains of mice such as those described by Kanasawa et al. (1984) Diabetologia, 27: 113. EAE in mouse and rat serves as a model for MS in human. In this model, the demyelinating disease is induced by administration of myelin basic protein (see Paterson (1986) Textbook of Immunopathology, Mischer et al., eds., Grune and Stratton, New York, pp. 179-213; McFarlin et al. (1973) Science, 179: 478: and Satoh et al. (1987) J. Immunol., 138: 179).

A ligand comprising a single variable domain, or composition thereof, which specifically binds vWF, e.g., human vWF, a vWF Al domain, the Al domain of activated vWF, or the vWF A3 domain, may further comprise a thrombolytic agent. This thrombolytic agent may be non-covalently or covalently attached to a single variable domain, in particular to an antibody single variable domain, via covalent or non-covalent means as known to one of skill in the art. Non-covalent means include via a protein interaction such as biotin/strepavidin, or via an immunoconjugate. Alternatively, the thrombolytic agent may be administered simultaneously, separately or sequentially with respect to a ligand that consists of or comprises a single variable domain that binds vWF or a vWF domain as described above, or a composition thereof. Thrombolytic agents according to the invention may include, for example, staphylokinase, tissue plasminogen activator, streptokinase, single chain streptokinase, urokinase and acyl plasminogen streptokinase complex.

Also described herein are invasive medical devices coated with a single variable domain, or a ligand comprising a single variable domain, or a composition thereof, or a single variable domain resulting from a screening method described herein. Non-limiting examples of devices include surgical tubing, occlusion devices, prosthetic devices. Application for said devices include surgical procedures which require a modulation of platelet-mediated aggregation around the site of invasion (e.g. a device coated with a single variable domain which specifically binds vWF) or a modulation of inflammation (e.g. a device coated with a single variable domain which specifically binds TNF alpha).

Generally, the present ligands will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The ligands of the present invention may be used as separately administered compositions or in conjunction with other agents. These can include various immunotherapeutic drugs, such as cylcosporine, methotrexate, adriamycin or cisplatinum, and immunotoxins. Pharmaceutical compositions can include “cocktails” of various cytotoxic or other agents in conjunction with the ligands of the present invention, or even combinations of ligands according to the present invention having different specificities, such as ligands selected using different target antigens or epitopes, whether or not they are pooled prior to administration.

The route of administration of pharmaceutical compositions according to the invention may be any of those commonly known to those of ordinary skill in the art. For therapy, including without limitation immunotherapy, the selected ligands thereof of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician.

As will be appreciated by the skilled artisan, the route and/or mode of administration will vary depending upon the desired results. In certain embodiments, the active compound can be prepared with a carrier that will protect the compound against rapid release, such as a controlled release formulation, including implants, transdermal patches, and microencapsulated delivery systems. Single domain antibody constructs are well suited for formulation as extended release preparations due, in part, to their small size—the number of moles per dose can be significantly higher than the dosage of, for example, full sized antibodies. BiodegradAble, biocompatible polymers can be used, such as ethylene vinyl acetate, polyanhydrides, polyglycolic acid, collagen, polyorthoesters, and polylactic acid. Prolonged absorption of injectable compositions can be brought about by including in the composition an agent that delays absorption, for example, monostearate salts and gelatin. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978. Additional methods applicable to the controlled or extended release of polypeptide agents such as the single immunoglobulin variable domain polypeptides disclosed herein are described, for example, in U.S. Pat. Nos. 6,306,406 and 6,346,274, as well as, for example, in U.S. Patent Application Nos. US20020182254 and US20020051808, all of which are incorporated herein by reference.

The ligands as described herein can be lyophilised for storage and reconstituted in a suitable carrier prior to use. This technique has been shown to be effective with conventional immunoglobulins and art-known lyophilisation and reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate.

The compositions containing the present ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of ligand, e.g. antibody, receptor (e.g. a T-cell receptor) or binding protein thereof per kilogram of body weight, with doses of 0.05 to 2.0 mg/kg/dose being more commonly used. For prophylactic applications, compositions containing the present ligands or cocktails thereof may also be administered in similar or slightly lower dosages.

Treatment performed using the compositions described herein is considered “effective” if one or more symptoms is reduced (e.g., by at least 10% or at least one point on a clinical assessment scale), relative to such symptoms present before treatment, or relative to such symptoms in an individual (human or model animal) not treated with such composition. Symptoms will obviously vary depending upon the disease or disorder targeted, but can be measured by an ordinarily skilled clinician or technician. Such symptoms can be measured, for example, by monitoring the level of one or more biochemical indicators of the disease or disorder (e.g., levels of an enzyme or metabolite correlated with the disease, affected cell numbers, etc.), by monitoring physical manifestations (e.g., inflammation, tumor size, etc.), or by an accepted clinical assessment scale, for example, the Expanded Disability Status Scale (for multiple sclerosis), the Irvine Inflammatory Bowel Disease Questionnaire (32 point assessment evaluates quality of life with respect to bowel function, systemic symptoms, social function and emotional status—score ranges from 32 to 224, with higher scores indicating a better quality of life), the Quality of Life Rheumatoid Arthritis Scale, or other accepted clinical assessment scale as known in the field. A sustained (e.g., one day or more, preferably longer) reduction in disease or disorder symptoms by at least 10% or by one or more points on a given clinical scale is indicative of “effective” treatment. Similarly, prophylaxis performed using a composition as described herein is “effective” if the onset or severity of one or more symptoms is delayed, reduced or abolished relative to such symptoms in a similar individual (human or animal model) not treated with the composition.

A composition containing a ligand or cocktail thereof according to the present invention may be utilised in prophylactic and therapeutic settings to aid in the alteration, inactivation, killing or removal of a select target cell population in a mammal. In addition, the selected repertoires of polypeptides described herein may be used extracorporeally or in vitro selectively to kill, deplete or otherwise effectively remove a target cell population from a heterogeneous collection of cells. Blood from a mammal may be combined extracorporeally with the ligands, e.g. antibodies, cell-surface receptors or binding proteins thereof whereby the undesired cells are killed or otherwise removed from the blood for return to the mammal in accordance with standard techniques.

### I: Use of Half-Life Enhanced Dual-Specific Ligands According to the Invention

Dual-specific ligands according to the method of the present invention, as well a ligands comprising one or more single variable domains as defined herein, may be employed in in vivo therapeutic and prophylactic applications, in vivo diagnostic applications and the like.

Therapeutic and prophylactic uses of dual-specific ligands prepared according to the invention, as well a ligands comprising one or more single variable domains as defined herein, involve the administration of ligands according to the invention to a recipient mammal, such as a human. Dual specific antibodies according to the invention as well a ligands comprising one or more single variable domains as defined herein, comprise at least one specificity for a half-life enhancing molecule; one or more further specificities may be directed against target molecules. For example, a dual-specific IgG may be specific for four epitopes, one of which is on a half-life enhancing molecule. Dual-specificity as well as tri-specificity as well as high valencies, can allow ligands comprising at least one single variable domain, to bind to multimeric antigen with great avidity. Dual-specific antibodies can allow the cross-linking of two antigens, for example in recruiting cytotoxic T-cells to mediate the killing of tumour cell lines.

Substantially pure dual-specific ligands according to the method of the present invention, as well a ligands comprising one or more single variable domains as defined herein, or binding proteins thereof, such as single variable domain monomers (i.e. dAb monomers), of at least 90 to 95% homogeneity are preferred for administration to a mammal, and 98 to 99% or more homogeneity is most preferred for pharmaceutical uses, especially when the mammal is a human. Once purified, partially or to homogeneity as desired, the ligands may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

Dual-specific ligands according to the method of the present invention, as well a ligands comprising one or more single variable domains as defined herein, will typically find use in preventing, suppressing or treating inflammatory states, allergic hypersensitivity, cancer, bacterial or viral infection, and autoimmune disorders (which include, but are preferably not limited to, Type I diabetes, multiple sclerosis, rheumatoid arthritis, systemic lupus erythematosus, Crohn's disease and myasthenia gravis).

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the dual specific ligands in protecting against or treating the disease are available. Methods for the testing of systemic lupus erythematosus (SLE) in susceptible mice are known in the art (Knight et al. (1978) J. Exp. Med., 147: 1653; Reinersten et al. (1978) New Eng. J. Med., 299: 515). Myasthenia Gravis (MG) is tested in SJL/J female mice by inducing the disease with soluble AchR protein from another species (Lindstrom et al. (1988) Adv. Immunol., 42: 233). Arthritis is induced in a susceptible strain of mice by injection of Type II collagen (Stuart et al. (1984) Ann. Rev. Immunol., 42: 233). A model by which adjuvant arthritis is induced in susceptible rats by injection of mycobacterial heat shock protein has been described (Van Eden et al. (1988) Nature, 331: 171). Thyroiditis is induced in mice by administration of thyroglobulin as described (Maron et al. (1980) J. Exp. Med., 152: 1115). Insulin dependent diabetes mellitus (IDDM) occurs naturally or can be induced in certain strains of mice such as those described by Kanasawa et al. (1984) Diabetologia, 27: 113. EAE in mouse and rat serves as a model for MS in human. In this model, the demyelinating disease is induced by administration of myelin basic protein (see Paterson (1986) Textbook of Immunopathology, Mischer et al., eds., Grune and Stratton, New York, pp. 179-213; McFarlin et al. (1973) Science, 179: 478: and Satoh et al. (1987) J. Immunol., 138: 179).

Dual specific ligands according to the invention and dAb monomers able to bind to extracellular targets involved in endocytosis (e.g. Clathrin) enable dual specific ligands to be endocytosed, enabling another specificity able to bind to an intracellular target to be delivered to an intracellular environment. This strategy requires a dual specific ligand with physical properties that enable it to remain functional inside the cell. Alternatively, if the final destination intracellular compartment is oxidising, a well folding ligand may not need to be disulphide free.

Generally, the present dual specific ligands will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The ligands of the present invention may be used as separately administered compositions or in conjunction with other agents. These can include various immunotherapeutic drugs, such as cylcosporine, methotrexate, adriamycin or cisplatinum, and immunotoxins. Pharmaceutical compositions can include “cocktails” of various cytotoxic or other agents in conjunction with the ligands of the present invention.

The route of administration of pharmaceutical compositions according to the invention may be any of those commonly known to those of ordinary skill in the art. For therapy, including without limitation immunotherapy, the ligands of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician.

The ligands of the invention can be lyophilised for storage and reconstituted in a suitable carrier prior to use. This technique has been shown to be effective with conventional immunoglobulins and art-known lyophilisation and reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate.

The compositions containing the present ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of ligand per kilogram of body weight, with doses of 0.05 to 2.0 mg/kg/dose being more commonly used. For prophylactic applications, compositions containing the present ligands or cocktails thereof may also be administered in similar or slightly lower dosages.

A composition containing a ligand according to the present invention may be utilised in prophylactic and therapeutic settings to aid in the alteration, inactivation, killing or removal of a select target cell population in a mammal.

In addition, the selected repertoires of polypeptides described herein may be used extracorporeally or in vitro selectively to kill, deplete or otherwise effectively remove a target cell population from a heterogeneous collection of cells. Blood from a mammal may be combined extracorporeally with the ligands, e.g. antibodies, cell-surface receptors or binding proteins thereof whereby the undesired cells are killed or otherwise removed from the blood for return to the mammal in accordance with standard techniques.

Selection and Characterisation of Ligands Comprising a Single Variable Domain for Binding to Serum Albumin from a Range of Species

A ligand can comprise one or more single variable domains, e.g., immunoglobulin single variable domains as well as non-immunoglobulin single variable domains, where at least one of the single variable domains specifically binds to serum albumin from human, as well as from non-human species. In one embodiment, the single variable domain specifically binds only serum albumin which is endogenous to a human. In another embodiment, the single variable domain specifically binds serum albumin from a non-human species. Alternatively, the single variable domain specifically binds both serum albumin which is endogenous to a human, as well as serum albumin which is endogenous to one or more non human species. As a nonlimiting example, such a single variable domain can specifically bind serum albumin endogenous to both human and cynomolgus, or serum albumin endogenous to both human and rat, or serum albumin from both human and mouse, or serum albumin from both human and pig. Alternatively, the single variable domain specifically binds to two or more serum albumin from two or more non-human species. As used herein, serum albumin can be expressed by a gene endogenous to a species, i.e. natural serum albumin, and/or by a recombinant equivalent thereof In one embodiment, the serum albumin includes fragments, analogs and derivatives of natural and recombinant serum albumin. Such fragments of serum albumin include fragments containing domain I, domain II, and/or domain III, or combinations of one or two or more of each of domains I, II and III of serum albumin, preferably human serum albumin. Domain II of serum albumin is preferred as a target for the single variable domain as defined herein. Other preferred combinations are Domain I and Domain II; Domain I and Domain III; Domain II and Domain III; and Domain I alone; Domain II alone; and Domain III alone; and Domain I and Domain II and Domain III. In one embodiment, the serum albumin is recombinant serum albumin exogenously expressed in a non-human host, such as an animal host, or a unicellular host such as yeast or bacteria.

The species from which the serum albumin is endogenous includes any species which expresses endogenous serum albumin, including, but preferably not limited to, the species of human, mouse, murine, rat, cynomolgus, porcine, dog, cat, horse, goat, and hamster. In some instances serum albumin endogenous to camel or lama are excluded.

The single variable domain can be an immunoglobulin single variable domain, including but preferably not limited to an antibody heavy chain single variable domain, an antibody VHH heavy chain single variable domain, a human antibody heavy chain single variable domain, a human VH3 heavy chain single variable domain, an antibody light chain single variable domain, a human antibody light chain single variable domain, a human antibody kappa light chain single variable domain, and/or a human lambda light chain single variable domain.

The single variable domain which specifically binds to serum albumin can be a single variable domain comprising an immunoglobulin scaffold or a non-immunoglobulin scaffold. The serum albumin binding, single variable domain can comprise one or two or three of CDR1, CDR2 and CDR3 from an antibody variable domain, preferably from a single variable domain, where the CDR region(s) is provided on a non-immunoglobulin scaffold, such as CTLA-4, lipocallin, staphylococcal protein A (SPA), GroEL and fibronectin, an Avimer™ and an Affibody™ scaffold. Alternatively, the serum albumin binding, non-immunoglobulin single variable domain can contain neither an antibody CDR region(s) nor a complete binding domain from an antibody. Alternatively, the serum albumin binding, single variable domain(s), can be single variable domains which comprise one or two or three of any of CDR1, CDR2 and CDR3 from an antibody variable domain, preferably a single variable domain; these CDR regions can be provided on a heavy or a light chain antibody framework region. Frameworks include, for example, VH frameworks, such as VH3 (including DP47, DP38 and DP45) and VHH frameworks described supra, as well as VL frameworks, including Vkappa (such as DPK9), and Vlambda frameworks. In some embodiments, the variable domain comprises at least one human framework region having an amino acid sequence encoded by a human germ line antibody gene segment, or an amino acid sequence comprising up to 5 amino acid differences relative to the amino acid sequence encoded by a human germ line antibody gene segment. In other embodiments, the variable domain comprises four human framework regions, FW1, FW2, FW2 and FW4, having amino acid sequences encoded by a human germ line antibody gene segment, or the amino acid sequences of FW1, FW2, FW3 and FW4 collectively containing up to 10 amino acid differences relative to the amino acid sequences encoded by the human germ line antibody gene segment. Preferably, all three CDR regions are provided on either an immunoglobulin scaffold (preferably heavy chain or light chain antibody scaffold) or a non-immunoglobulin scaffold as defined herein, either of which can be non-human, synthetic, semi-synthetic. Alternatively, any combination of one, two or all three of CDR1, CDR2 and/or CDR3 regions are provided on either the immunoglobulin scaffold or the non-immunoglobulin scaffold, for example, either the CDR3 region alone, or the CDR2 and CDR3 regions together, or the CDR1 and CDR2 are provided on either the immunoglobulin scaffold or the non-immunoglobulin scaffold. Suitable scaffolds and techniques for such CDR grafting will be clear to the skilled person and are well known in the art, see for example U.S. application Ser. No. 07/180,370, WO 01/27160, EP 0 605 522, EP 0 460 167, U.S. application Ser. No. 07/054,297, Nicaise et al., Protein Science (2004), 13:1882-1891; Ewert et al., Methods, 2004 October; 34(2):184-199; Kettleborough et al., Protein Eng. 1991 October; 4(7): 773-783; O′Brien and Jones, Methods Mol. Biol. 2003: 207: 81-100; and Skerra, J. Mol. Recognit. 2000: 13: 167-187, and Saerens et al., J. Mol. Biol. 2005 Sep. 23; 352(3):597-607, and the further references cited therein.

The ligands can comprise one or more of such single variable domains which specifically bind serum albumin, preferably comprising at least one single variable domain which specifically binds both serum albumin which is endogenous to humans and at least one additional serum albumin which is endogenous to a non-human species. In one embodiment, this single variable domain specifically binds to serum albumin which is endogenous to human with a Kd value which is within 10 fold of the Kd value with which it specifically binds (i.e. cross reacts with) to at least one serum albumin which is endogenous to a non-human species. Alternatively this single variable domain specifically binds to serum albumin which is endogenous to human with a Kd value which is within 15, 20, 25, 30, 50 or up to approximately 100 fold of the Kd value with which it specifically binds (i.e. cross reacts with) to at least one serum albumin which is endogenous to a non-human species. In some embodiments the Kd can range from 300 nM to about 5 pM. In other embodiments, the single variable domain specifically binds to serum albumin with a Koff of about 5×10⁻¹ S⁻¹ or less.

In one embodiment, this single variable domain specifically binds to serum albumin which is endogenous to a first non-human species with a Kd value which is within 10 fold of the Kd value with which it specifically binds to (i.e. cross reacts to) at least one serum albumin which is endogenous to a second non-human species. Alternatively, this single variable domain specifically binds to serum albumin which is endogenous to the first non-human species with a Kd value which is within 15, 20, 25, 30, 50 or up to approximately 100 fold of the Kd value with which it specifically binds to (i.e. cross reacts to) to at least one serum albumin which is endogenous to the second non-human species. In some embodiments, the Kd can range from 300 nM to about 5 pM. In other embodiments, the single variable domain specifically binds to serum albumin with a K_{off} of at least 5×10⁻¹, S⁻¹, 5×10⁻² S⁻¹, 5×10⁻³ S⁻¹, 5×10⁻⁴ S⁻¹, 5'10⁻⁶ S⁻¹, 5×10⁻⁷ S⁻¹, 5×10⁻⁸ S⁻¹, 5×10⁻⁹ S⁻¹, 5×10⁻¹⁰ S⁻¹, or less, preferably with a K_{off} ranging from 1×10⁻⁶ S⁻¹ to 1×10⁻⁸ S⁻¹.

For example, such a ligand can include an immunoglobulin single variable domain, where the immunoglobulin single variable domain specifically binds to human serum albumin and mouse serum albumin, and where the T beta half life of the ligand is substantially the same as the T beta half life of mouse serum albumin in a mouse host. In one version of such a ligand, the epitope binding domain contains a non-immunoglobulin scaffold which specifically binds to human serum albumin and mouse serum albumin, and wherein the T beta half life of the ligand is substantially the same as the T beta half life of mouse serum albumin in a mouse host. The phrase “substantially the same” means that the ligand has a T beta half life in a mouse host that is at least 50% that of mouse serum albumin in a mouse host, that is at least 55%, 60%, 65%, 70%, 75%, 80%, 85%, 90%, 92%, 94%, 95%, 96%, 97%, 98%, 99%, 100%, 101%, 102%, 105%, 110%, 125%, and up to 150% that of the T beta half life of mouse serum albumin in a mouse host. The non-immunoglobulin scaffold can optionally include fragments of an antibody single variable domain, such as one or more of the CDR regions of an antibody variable domain, including an antibody single variable domain that has a T beta half life in a human host that is at least 50%, 55%, 60%, 65%, 70%, 75%, 80%, 85%, 90%, 92%, 94%, 95%, 96%, 97%, 98%, 99%, 101%, 102%, 105%, 110%, 125%, or up to 150% that of the T beta half life of human serum albumin in a human host.

For example, one embodiment is a single variable domain, where the single variable domain specifically binds to human serum albumin and rat serum albumin, and where the T beta half life of the ligand is substantially the same as the T beta half life of rat serum albumin in a rat host. In one version of such a ligand, the single variable binding domain contains a non-immunoglobulin scaffold which specifically binds to human serum albumin and rat serum albumin, and wherein the T beta half life of the ligand is substantially the same as the T beta half life of rat serum albumin in a rat host. The phrase “substantially the same” means that the ligand has a T beta half life in a rat host that is at least 50% that of rat serum albumin in a rat host, that is up to 55%, 60%, 65%, 70%, 75%, 80%, 85%, 90%, 92%, 94%, 95%, 96%, 97%, 98%, 99%, 100%, 101%, 102%, 105%, 110%, 125%, up to 150% that of the T beta half life of rat serum albumin in a rat host. The non-immunoglobulin scaffold can optionally include fragments of an antibody single variable domain, such as one or more of the CDR regions of an antibody variable domain.

For example, a ligand can include an immunoglobulin single variable domain, where the immunoglobulin single variable domain specifically binds to human serum albumin and porcine serum albumin, and where the T beta half life of the ligand is substantially the same as the T beta half life of porcine serum albumin in a porcine host. In one version of a ligand, the epitope binding domain contains a non-immunoglobulin scaffold which specifically binds to human serum albumin and porcine serum albumin, and wherein the T beta half life of the ligand is substantially the same as the T beta half life of porcine serum albumin in a porcine host. The phrase “substantially the same” means that the ligand has a T beta half life in a porcine host that is at least 50% that of porcine serum albumin in a porcine host, that is up to 55%, 60%, 65%, 70%, 75%, 80%, 85%, 90%, 92%, 94%, 95%, 96%, 97%, 98%, 99%, 100%, 101%, 102%, 105%, 110%, 125%, up to 150% that of the T beta half life of porcine serum albumin in a porcine host. The non-immunoglobulin scaffold can optionally include fragments of an antibody single variable domain, such as one or more of the CDR regions of an antibody variable domain, including an antibody single variable domain.

For example, a ligand can include an immunoglobulin single variable domain, where the immunoglobulin single variable domain specifically binds to human serum albumin and cynomolgus serum albumin, and where the T beta half life of the ligand is substantially the same as the T beta half life of cynomolgus serum albumin in a cynomolgus host. In one version of a ligand, the domain that binds serum albumin contains a non-immunoglobulin scaffold which specifically binds to human serum albumin and cynomolgus serum albumin, and wherein the T beta half life of the ligand is substantially the same as the T beta half life of cynomolgus serum albumin in a cynomolgus host. The phrase “substantially the same” means that the ligand has a T beta half life in a cynomolgus host that is at least 50% that of cynomolgus serum albumin in a cynomolgus host, that is up to 55%, 60%, 65%, 70%, 75%, 80%, 85%, 90%, 92%, 94%, 95%, 96%, 97%, 98%, 99%, 100%, 101%, 102%, 105%, 110%, 125%, or up to 150% that of the T beta half life of cynomolgus serum albumin in a cynomolgus host.

The non-immunoglobulin scaffold can optionally include fragments of an antibody single variable domain, such as one or more of the CDR regions of an antibody variable domain.

In one embodiment, a ligand and/or dual specific ligand contains a single variable domain which specifically binds to serum albumin that is endogenous to human, has a T beta half life in a human host that is at least 50% that of human serum albumin in a human host, up to 55%, 60%, 65%, 70%, 75%, 80%, 85%, 90%, 92%, 94%, 95%, 96%, 97%, 98%, 99%, 100%, 101%, 102%, 105%, 110%, 125% or up to 150% that of the T beta half life of human serum albumin in a human host. In a preferred embodiment, the single variable domain which specifically binds to serum albumin that is endogenous to a non-human, has a T beta half life in its respective non-human host that is at least 50% that of the non human serum albumin in its respective non-human host, up to 55%, 60%, 65%, 70%, 75%, 80%, 85%, 90%, 92%, 94%, 95%, 96%, 97%, 98%, 99%, 100%, 101%, 102%, 105%, 110%, 125%, or up to 150% that of the T beta half life of the non-human serum albumin in its respective non-human host. In a preferred embodiment, the single variable domain which specifically binds to serum albumin that is endogenous to human, and which also specifically binds specifically to serum albumin from at least one non-human species, has a T beta half life in a human host that is up to 55%, 60%, 65%, 70%, 75%, 80%, 85%, 90%, 92%, 94%, 95%, 96%, 97%, 98%, 99%, 101%, 102%, 105%, 110%, 125%, or up to 150% of human serum albumin in a human host, and a T beta half life in the non-human host that is up to 55%, 60%, 65%, 70%, 75%, 80%, 85%, 90%, 92%, 94%, 95%, 96%, 97%, 98%, 99%, 100%, 101%, 102%, 105%, 110%, 125%, or up to 150% of the non-human serum albumin in its respective non-human host. In some embodiments, the T beta half life of the single variable domain which specifically binds to serum albumin can range from as low as 2 hours up to and including 3 hours, 4 hours, 5 hours, 6 hours, 7 hours, 8 hours, 9 hours, 10 hours, 11 hours, 12 hours, 14 hours, 16 hours, 18 hours, 20 hours, 22 hours, 1 day, 2 days, 3 days, 4 days, 4 days, 6 days, 8 days, 10 days, 12 days, 14 days, 16 days, 18 days, up to as high as 21 days or more. In a human host, as well as a non-human host such as a porcine, cynomulgus, rat, murine, mouse host, the T beta half life of the single variable domain which specifically binds to serum albumin can range from as low as 2 hours up to and including 3 hours, 4 hours, 5 hours, 6 hours, 7 hours, 8 hours, 9 hours, 10 hours, 11 hours, 12 hours, 14 hours, 16 hours, 18 hours, 20 hours, 22 hours, 1 day, 2 days, 3 days, 4 days, 4 days, 6 days, 8 days, 10 days, 12 days, 14 days, 16 days, 18 days, up to as high as 21 days, or more. Other preferred T beta half lives of a ligand comprising a single variable domain which specifically binds to serum albumin include: in a monkey host from about 3 to about 5, 6, 7, or 8 days, including from as low as 2 hours, up to and including 3 hours, 4 hours, 5 hours, 6 hours, 7 hours, 8 hours, 9 hours, 10 hours, 11 hours, 12 hours, 14 hours, 16 hours, 18 hours, 20 hours, 22 hours, 1 day, 2 days, 3 days, 4 days, 4 days, 6 days, 8 days, 10 days, 12 days, 14 days, 16 days, 18 days, up to as high as 21 days. In a rat or mouse host, the T beta half life of the single variable domain which specifically binds to serum albumin can range from as low as 40 hours to as high as about 75 hours, and includes as low as 2 hours up to and including 3 hours, 4 hours, 5 hours, 6 hours, 7 hours, 8 hours, 9 hours, 10 hours, 11 hours, 12 hours, 14 hours, 16 hours, 18 hours, 20 hours, 22 hours, 1 day, 2 days, 3 days, 4 days, 4 days, 6 days, 8 days, 10 days, 12 days, 14 days, 16 days, 18 days, up to as high as 21 days.

The single variable domain which specifically binds to serum albumin includes Vkappa single variable domains, selected from, but preferably not limited to DOM7h-9 DOM7h-1, DOM7h-8, DOM7h-9, DOM7h-11, DOM7h-12, DOM7h-13 and DOM7h-14. DOM7r-3 and DOM7r-16, and/or those domains which compete for binding serum albumin, preferably human serum albumin, with the single variable domains selected from, but preferably not limited to, dAb7r20, dAb7r21, dAb7r22, dAb7r23, dAb7r24, dAb7r25, dAb7r26, dAb7r27, dAb7r28, dAb7r29, dAb7r30, dAb7r31, dAb7r32, dAb7r33, dAb7h21, dAb7h22, dAb7h23, Ab7h24, Ab7h25, Ab7h26, dAb7h27, dAb7h30 dAb7h31, dAb7m12, dAb7m16, dAb7m26, dAb7r1, dAb7r3, dAb7r4, dAb7r5, dAb7r7, dAb7r8, dAb7r13, dAb7r14, dAb7rl5, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7p1, and dAb7p2. The single variable domain which specifically binds to serum albumin can be an antibody heavy chain single variable domain, in particular, human VH₃, or VHH. An afore-mentioned single variable domain may also additionally specifically bind human serum albumin with a K_{off} of at least 5×10⁻¹, S⁻¹, 5×10⁻² S⁻¹, 5×10⁻³ S⁻¹, 5×10⁻⁴ S⁻¹, 5×10⁻⁵ S⁻¹, 5×10⁻⁶ S⁻¹, 5×10⁻⁷ S⁻¹, 5×10⁻⁸ S⁻¹, 5×10⁻⁹ S⁻¹, 5×10⁻¹⁰ S⁻¹, or less, preferably with a K_{off} ranging from 1×10⁻⁶ S⁻¹ to 1×10⁻⁸ S⁻¹. Single variable domains that specifically bind human serum albumin and a serum albumin that is endogenous to a non human species, can further bind a serum albumin that is endogenous to a third, fourth, fifth, sixth, seventh, eighth, ninth or tenth non human species. In one nonlimiting embodiment, the single variable domain which specifically binds to human serum albumin and rat serum albumin, further specifically binds to cynomolgus serum albumin. In another nonlimiting embodiment, the single variable domain which specifically binds to human serum albumin and mouse serum albumin, further specifically binds to cynomolgus serum albumin.

As described herein, a ligand which contains one single variable domain (monomer) or more than one single variable domains (multimer, fusion protein, conjugate, and dual specific ligand as defined herein) which specifically binds to serum albumin, can further comprise one or more entities selected from, but preferably not limited to a label, a tag , an additional single variable domain, a dAb, an antibody, and antibody fragment, a marker and a drug. One or more of these entities can be located at either the COOH terminus or at the N terminus or at both the N terminus and the COOH terminus of the ligand comprising the single variable domain, (either immunoglobulin or non-immunoglobulin single variable domain). One or more of these entities can be located at either the COOH terminus, or the N terminus, or both the N terminus and the COOH terminus of the single variable domain which specifically binds serum albumin of the ligand which contains one single variable domain (monomer) or more than one single variable domains (multimer, fusion protein, conjugate, and dual specific ligand as defined herein). Non-limiting examples of tags which can be positioned at one or both of these termini include a HA, his or a myc tag. The entities, including one or more tags, labels and drugs, can be bound to the ligand which contains one single variable domain (monomer) or more than one single variable domain (multimer, fusion protein, conjugate, and dual specific ligand as defined herein), which binds serum albumin, either directly or through linkers as described in a separate section below.

A ligand which contains one single variable domain (monomer) or more than one single variable domains (multimer, fusion protein, conjugate, and dual specific ligand as defined herein) which specifically binds to serum albumin, or which specifically binds both human serum albumin and at least one non-human serum albumin, can specifically bind to one or more of Domain I, and/or Domain II and/or domain III of human serum albumin, as described further below. In addition to containing one or more single variable domains, (for example, a serum albumin binding immunoglobulin single variable domain or a serum albumin binding non-immunoglobulin single variable domain) which specifically binds to a serum albumin, such as human serum albumin, or which specifically binds both human serum albumin and at least one non-human serum albumin, the ligand can contain one or more additional domains capable of specifically binding an antigen and/or epitope other than serum albumin, the antigen or epitope being selected from the group consisting of any animal protein, including cytokines, and/or antigens derived from microorganisms, pathogens, unicellular organisms, insects, viruses, algae and plants. These one or more additional domain(s) which bind a moiety other than serum albumin can be a non-immunoglobulin binding domain, a non-immunoglobulin single variable domain, and/or an immunoglobulin single variable domain.

In some embodiments, a dual specific ligand which contains one or more single variable domains (either an immunoglobulin single variable domain or a non-immunoglobulin single variable domain) which specifically binds to a serum albumin, such as human serum albumin, or which specifically binds both human serum albumin and at least one non-human serum albumin, can be composed of (a) the single variable domain that specifically binds serum albumin and a single variable domain that specifically binds a ligand other than serum albumin, both of the single variable domains being a heavy chain single variable domain; or (b) the single variable domain that specifically binds serum albumin and a single variable domain that specifically binds a ligand other than serum albumin, both of the single variable domains being a light chain single variable domain; or (c) the single variable domain that specifically binds serum albumin is a heavy chain single variable domain, and the single variable domain that specifically binds an antigen other than serum albumin is a light chain single variable domain; or (d) the single variable domain that specifically binds serum albumin is a light chain single variable domains, and the single variable domain that specifically binds an antigen other than serum albumin is a heavy chain single variable domain.

Also encompassed herein is an isolated nucleic acid encoding any of the ligands described herein, e.g., a ligand which contains one single variable domain (monomer) or more than one single variable domains (e.g., multimer, fusion protein, conjugate, and dual specific ligand as defined herein) which specifically binds to serum albumin, or which specifically binds both human serum albumin and at least one non-human serum albumin, or functionally active fragments thereof. Also encompassed herein is a vector and/or an expression vector thereof, a host cell comprising the vector, e.g., a plant or animal cell and/or cell line transformed with a vector, a method of expressing and/or producing one or more ligands which contains one single variable domain (monomer) or more than one single variable domains (e.g., multimer, fusion protein, conjugate, and dual specific ligand as defined herein) which specifically binds to serum albumin, or fragment(s) thereof encoded by said vectors, including in some instances culturing the host cell so that the one or more ligands or fragments thereof are expressed and optionally recovering the ligand which contains one single variable domain (monomer) or more than one single variable domains (e.g., multimer, fusion protein, conjugate, and dual specific ligand as defined herein) which specifically binds to serum albumin, from the host cell culture medium. Also encompassed are methods of contacting a ligand described herein with serum albumin, including serum albumin and/or non-human serum albumin(s), and/or one or more targets other than serum albumin, where the targets include biologically active molecules, and include animal proteins, cytokines as listed above, and include methods where the contacting is in vitro as well as administering any of the ligands described herein to an individual host animal or cell in vivo and/or ex vivo. Preferably, administering ligands described herein which comprises a single variable domain (immunoglobulin or non-immunoglobulin) directed to serum albumin and/or non-human serum albumin(s), and one or more domains directed to one or more targets other than serum albumin, will increase the ligand's half life, including the T beta half life, of the ligand. Nucleic acid molecules encoding the single domain containing ligands or fragments thereof, including functional fragments thereof, are described herein. Vectors encoding the nucleic acid molecules, including but preferably not limited to expression vectors, are described herein, as are host cells from a cell line or organism containing one or more of these expression vectors. Also described are methods of producing any the single domain containing ligands, including, but preferably not limited to any of the aforementioned nucleic acids, vectors and host cells.

Epitope Mapping of Serum Albumin

Serum albumins from mammalian species have a similar structure, containing three predominate domains with a similar folding and disulphide bonding pattern, as highlighted in FIG. 46. The protein is believed to have arisen from two tandem duplication events, and subsequent diversification of residues.

The structure of human serum albumin has been solved by X-ray crystallography, with/without a variety of bound ligands:
Atomic structure and chemistry of human serum albumin. He X M, Carter D C.
Nature. 1992; 358: 209-15. Erratum in: Nature 1993; 364: 362.
Atomic structure and chemistry of human serum albumin. He X M, Carter D C; J Mol Biol. 2001; 314: 955-60.
Crystal structures of human serum albumin complexed with monounsaturated and polyunsaturated fatty acids. Petitpas I, Grune T, Bhattacharya A A, Curry S.; J Biol Chem. 2001;276: 22804-9.

Human serum albumin has been shown to be a heart shaped molecule. The individual domains, termed I, II and III, are predominantly helical, and are each composed of two sub-domains, termed IA, IB, IIA, 2B, IIIA, and IIIB. They are linked by flexible, random coils.

Described herein is a ligand which contains one or more single variable domains which specifically binds to Domain H of human serum albumin. The single variable domain can be a VH antibody single variable domain. The single variable domain can be a VHH antibody single variable domain. The VH single variable domain can be a VH3 single variable domain. The VH3 single variable domain can be a human VH3 single variable domain. The ligand can alternatively, or additionally include a single variable domain which is a VKappa antibody single variable domain, including one of the following: DOM7h-1, DOM7h-8, DOM7h-9, DOM7h-11, DOM7h-12, DOM7h-13, DOM

The antibody single variable domain can include a set of four Kabat framework regions (FRs), which are encoded by antibody VH, preferably a VH3, framework germ line antibody gene segments. The VH3 framework is selected from the group consisting of DP47, DP38 and DP45. The antibody single variable domain can include a set of four Kabat framework regions (FRs) which are encoded by an antibody VL framework, preferably a VKappa framework, germline antibody gene segment. Preferably, the Kappa framework is DPK9.

The ligand which contains one or more single variable domains which specifically bind to Domain II of human serum albumin can further include one or more domains capable of specifically binding a moiety other than serum albumin, and can further comprise one or more entities including one or more of a label, a tag and a drug. The one or more domains capable of specifically binding a moiety other than serum albumin can be an immunoglobulin single variable domain. Also described herein is a ligand which contains one or more single variable domains which specifically binds to Domain II of human serum albumin, the domain including a non-immunoglobulin scaffold and CDR1, CDR2 and/or CDR3 regions, or where at least one of the CDR1, CDR2 and/or CDR3 regions is from a single variable domain of an antibody single variable domain that binds Domain II of human serum albumin. Non-immunoglobulin scaffolds include, but preferably are not limited to, CTLA-4, lipocallin, staphylococcal protein A (SPA), Affibody™, Avimers™, GroEL and fibronectin.

The ligand which contains one or more single variable domains which specifically binds to Domain II of human serum albumin includes those domains which specifically bind human serum albumin with a Kd of less than or equal to 300 nM. The ligand which contains one or more single variable domains which specifically binds to Domain II of human serum albumin can further comprise one or more entities including one or more of a label, a tag and a drug. The tag can include one or more of C-terminal HA or myc tags or N terminal HA or myc tags.

The ligand which contains one or more single variable domains which specifically binds to Domain II of human serum albumin, and which can further include one or more domains capable of specifically binding a moiety other than serum albumin, and which can optionally further comprise one or more entities including one or more of a label, a tag and a drug, can bind, through at least one of its single variable domains, an antigen including, but preferably not limited to a cytokine receptor, EPO receptor, ApoE, Apo-SAA, BDNF, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a), IL-9, IL-10, IL-11, IL-12, IL-13, IL-15, IL-16, IL-17, IL-18 (IGIF), Inhibin α, Inhibin β, IP-10 keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a), MIG, MLP1α, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumor necrosis factor (TNF), TNF-α, TNF-β, TNF receptor 1, TNF receptor II, TNIL-1, TPO, VEGF, VEGF receptor 1, VEGF receptor 2, VEGF receptor 3, GCP-2, GRO/MGSA, GRO-β, GRO-γ, HCC1, I-309, HER 1, HER 2, HER3 and HER4, CD4, human chemokine receptors CXCR4 or CCR5, non-structural protein type 3 (NS3) from the hepatitis C virus, TNF-alpha, IgE, IFN-gamma, MMP-12, CEA, H. pylori, TB, influenza, Hepatitis E, MMP-12, internalising receptors that are over-expressed on certain cells, such as the epidermal growth factor receptor (EGFR), ErBb2 receptor on tumor cells, an internalising cellular receptor, LDL receptor, FGF2 receptor, ErbB2 receptor, transferrin receptor, PDGF receptor, VEGF receptor, PsmAr, an extracellular matrix protein, elastin, fibronectin, laminin, α1-antitrypsin, tissue factor protease inhibitor, PDK1, GSK1, Bad, caspase-9, Forkhead, an of an antigen of Helicobacter pylori, an antigen of Mycobacterium tuberculosis, and an antigen of influenza virus.

The ligand which contains one or more single variable domains which specifically binds to Domain II of human serum albumin, and which can further include one or more domains capable of specifically binding a moiety other than serum albumin, is minimally a dual specific ligand, which can have one of the following structures: (a) each said single variable domain that specifically binds to Domain II of serum albumin and said single variable domain that specifically binds a moiety other than serum albumin, is an antibody heavy chain single variable domain; or (b) each said single variable domain that specifically binds to Domain II of serum albumin and said single variable domain that specifically binds a moiety other than serum albumin, is an antibody light chain single variable domain; or (c) said single variable domain that specifically binds to Domain II of serum albumin is an antibody heavy chain single variable domain, and said single variable domain that specifically binds an antigen other than serum albumin is an antibody light chain single variable domain; or (d) said single variable domain that specifically binds to Domain II of serum albumin is an antibody light chain single variable domain, and said single variable domain that specifically binds an antigen other than serum albumin is an antibody heavy chain single variable domain. Nucleic acid molecules encoding any ligands or fragments thereof, including functional fragments thereof, described herein, vectors including but preferably not limited to expression vectors, and host cells of any type cell line or organism, containing one or more of these expression vectors is included, and/or are methods of producing any ligands, including, but preferably not limited to any the aforementioned nucleic acids, vectors and host cells.

Serum albumin has a long serum half-life compared with other serum proteins, together with a positive relationship between serum concentration and fractional catabolic rates (i.e. the higher the concentration of SA, the higher the amount degraded), a property that it shares with IgG. It has recently emerged that both IgG and serum albumin share a recycling mechanism, mediated by the neonatal Fc receptor FcRn. FcRn is a type I MHC family member, composed of a heterodimer of the membrane anchored FCRGT chain, and non-membrane-bound beta-2 microglobulin. Mouse knockout mutants of either FcRn or beta-2 microglobulin express no functional FcRn, and exhibit an increased biosynthesis rate of serum albumin (˜20% increase), and an increased catabolism of serum albumin, leading to a 40% lower serum level of serum albumin, with a shorter half-life (Chaudhry et al 2005). In humans, mutations in beta-2 microglobulin have been shown give much reduced functional FcRn levels and ultimately to IgG deficiency and hypoalbuminaemia, characterised by a reduced serum half-life of HSA (Wani et al 2006, PNAS).

Though not wishing to be bound by theory, the proposed mechanism for FcRn-mediated salvage is as follows:
1. Plasma proteins are pinocytosed by cells of the endothelium lining all blood vessels, and perhaps pinocytotically active cells of the extravascular compartment. This is a non-specific step, and all proteins in circulation will be taken up. FcRn has a very low affinity for albumin (and IgGs) at serum pH, around pH 7.4.
2. Once pinocytosed, the vesicle formed acidifies to pH 5.0. Under acid conditions, FcRn has a higher affinity for albumin, and binds albumin, and also IgG. Albumin and IgG are thus bound to the FcRn receptor. FcRn binds human serum albumin at a site on Domain III, via a distinct site from that which binds IgG.
3. A sorting event occurs, by which the majority of non-receptor bound proteins are sorted into an endosome, where most proteins will be targeted for degradation. The receptor bound albumin and IgG are sorted into a vesicle targeted for the cell surface, and thus spared from degradation.
4. The cell surface targeted vesicle then either fuses with the cell surface, or briefly fuses with the cell membrane. Under these conditions, the pH of the endosome increases to approach pH 7.4, the FcRn affinity for albumin is reduced, and albumin is released back into the circulation.

We can therefore define a clear set of desirable parameters for any SA binding protein to have maximum half life. These parameters can be clearly exemplified using the serum albumin salvage receptor FcRn as a model, although will also apply to other receptors mediating a prolonged half life.
The affinity of the serum albumin binding will preferably be such that the SA binding protein does not dissociate from albumin while undergoing glomerular filtration in the kidney, thus minimising loss to the urine.
The binding to SA will preferably not have a detrimental effect on the binding of serum albumin to any receptors responsible for the maintenance of serum albumin levels in the circulation, as this would inhibit recycling, and hence reduce the half-life of both the serum albumin and the SA binder. Thus SA binding dAbs should bind a distinct epitope from that bound by FcRn on HSA domain HI, and the SA/dAb complex should also be capable of engaging FcRn.
The binding to SA will preferably be maintained under the conditions under which the receptor and bound SA/SA binder complex are sorted or recycled. Endosomal pH has been shown to approach pH 5.0, therefore stable binding of the dAb to serum albumin at both pH7.4 and pH 5.0 is desirable.
   As illustrated in Example 15 below, the majority of dAbs bind to the 2^{nd} domain of HSA and are therefore not expected to compete with binding of human serum albumin to FcRn. Two dAbs (DOM7h-27 and DOM7h-30) bind to Domain III.

An anti-SA DAb that retains sufficient affinity for SA in a pH range of 7.4 to 5.0.

In addition to affinity for SA, as well as in the absence of competition with the formation of SA:FcRn complexes, the serum-albumin-specific dAbs will preferably maintain affinity to SA within a pH range from pH 7.4 in the serum to pH 5.0 in the endosome to obtain full benefit of the FcRn-mediated salvage pathway.

In this pH range, only histidine residues and amino acid side-chains with perturbed pKa are likely to change their protonation state. If amino acid side-chains make a significant contribution to the binding energy of the complex, one could expect that a pH shift from one extreme to the other extreme in the range could result in lowering the binding affinity of the complex. Though not wishing to be bound by theory, this in turn would result in increasing the likelihood that the SA-specific dAb enters in the degradation pathway rather than being rescued through the FcRn-mediated salvage pathway.

Thus, for a SA binding AlbudAb™ (a dAb which specifically binds serum albumin), it is desirable to select one where the binding characteristics to serum albumin do not significantly change with pH (in the range of 5.0 to 7.4). A straightforward method to ensure this would be to analyze the amino acid sequences of the anti-SA dAbs for the absence of histidine residues in the CDRs. As shown below, several selection procedures for such a property can be envisaged:

For example, a first selection round is performed with the ‘naïve’ dAb phage repertoire using immobilized human serum albumin in conditions where the pH of the buffer is at pH 7.4 (e.g. PBS). The recovered and amplified phage population is then submitted to a second round of selection where the incubation buffer is at pH 5.0. The alternation of buffers and pHs are optionally repeated in further rounds in order to maintain selection pressure for dAb binding to HSA at both pHs.

In a second example, all selection rounds are performed with the ‘naïve’ dAb phage repertoire using immobilised human serum albumin in conditions where the pH of the buffer is at pH 7.4 (e.g. PBS). However, just after washing away unbound phage with PBS (or PBS supplemented with Tween) and prior to elution of bound phage, there is added an additional wash/incubation step at pH 5.0 for a prolonged period of time (e.g. up to 4 hours). During this period, phage displaying dAbs that are unable to bind SA at pH 5.0 (but able to bind at pH 7.4) are detached from the immobilised SA. After a second series of wash steps (at pH 5.0 with(out) Tween, bound phage is recovered and analysed.

In a third example, all selection rounds are performed with the ‘naïve’ dAb phage repertoire using immobilized human serum albumin in conditions where the pH of the buffer is at pH 7.4 (e.g. PBS). Best dAb candidates (i.e. able to bind at pH 7.4 and pH 5.0) are then identified by screening. Typically, the genes encoding dAbs are recovered from the pooled selected phage, subcloned into an expression vector that directs the soluble dAb in the supernatant of E. coli cultures. Individual clones are picked, grown separately in the wells of microtiter plates, and induced for expression. Supernatants (or purified dAbs) are then directly loaded onto a BIAcore chip to identify those dAbs displaying affinity for the immobilised serum albumin. Each supernatant is screened for binding (mainly the off-rate trace of the sensorgram) to HSA in conditions where the ‘running’ buffer is either at pH 7.4 or at pH 5.0. It should be noted that screening of dAb binding on the BIAcore would also be used as a preferred method to identify best leads from the two above examples.

Described herein is a ligand comprising a single variable domain as defined herein, where the single variable domain specifically binds serum albumin both at a natural serum pH, and at an intracellular vesicle pH. The natural serum pH is about 7.4, and wherein said intracellular vesicle pH can range from about 4.8 to 5.2, or can be at a pH of about 5. In one embodiment, the single variable domain can specifically binds serum albumin with a pH range of about 7 to 5, or can be at a pH of 7.4. Though not wishing to be bound by theory, a further characteristic of this ligand is that the its single variable domain that specifically binds serum albumin does not substantially dissociate from serum albumin while undergoing glomerular filtration in the kidney. Though not wishing to be bound by theory, a further characteristic of this ligand is that its single variable domain that specifically binds serum albumin does not substantially interfere with the binding of FcRn to the serum albumin. This single variable domain can be an antibody single variable domain; the antibody single variable domain can be a VH3 domain and/or the antibody single variable domain can be a V kappa domain. This single variable domain can comprise a non-immunoglobulin scaffold, e.g., CTLA-4, lipocallin, SpA, Affibody™, GroEL, Avimer™ or fibronectin scaffolds, and can contain one or more of CDR1, CDR2 and/or CDR3 from an antibody single variable domain that preferably, though not necessarily, specifically binds serum albumin. The single variable domain(s) of this ligand, can specifically bind human serum albumin, and/or including serum albumin from one or more species, e.g., human, rat, monkey, procine, rabbit, hamster, mouse or goat. The intracellular compartment can be any intracellular compartment of any cell of any animal, including an endosomal compartment or intracellular vesicle or a budding vesicle. The endosomal compartment can have a pH of about 5, or 5.0. The ligands described herein can contain one or more single variable domains including immunoglobulin and/or non-immunoglobulin domains where the binding of serum albumin to the single variable domain does not substantially competitively inhibit the binding of FcRn to serum albumin. These one or more singular variable domains can preferably specifically bind serum albumin with an equilibrium dissociation constant (Kd) of less than or equal to 300 nM.

Described herein is a method for selecting for a ligand comprising a single variable domain, which contains one single variable domain (monomer), or more than one single variable domains (e.g., multimer, fusion protein, conjugate, and dual specific ligand as defined herein) which specifically binds to serum albumin, where the single variable domain specifically binds human serum albumin at a natural serum pH, and where the single variable domain does not competitively inhibit the binding of human serum albumin to FcRn, and where the single variable domain specifically binds human serum albumin at a pH of an intracellular compartment, comprising the steps of: (A) selecting for ligands comprising a single variable domain which does not bind the regions of human serum albumin that bind FcRn, (B) from the ligands selected in step (A), selecting for ligands comprising a single variable domain which binds serum albumin at said natural serum pH. (C) selecting the ligands selected in step (B) for those which bind serum albumin at the pH of said intracellular compartment. Alternatively steps (A) and (B) can be reversed as follows: (A) selecting ligands comprising a single variable domain which binds human serum albumin at said natural serum pH, (B) from the ligands selected in (A), selecting ligands comprising a single variable domain which binds human serum albumin outside the regions of HSA that bind FcRn, and (C) from the ligands selected in step (B), selecting for those which bind serum albumin at said pH of said intracellular compartment. Also described is a method for selecting for a ligand comprising a single variable domain, where the single variable domain specifically binds human serum albumin at a natural serum pH, wherein the single variable domain does not competitively inhibit the binding of human serum albumin to FcRn, and where the single variable domain specifically binds human serum albumin at a pH of an intracellular compartment, comprising the steps of: (A) selecting for ligands comprising a single variable domain which does not bind the regions of human serum albumin that bind FcRn, (B) from step (A) selecting for ligands comprising a single variable domain which binds serum albumin at said natural serum pH, and (C) genetically modifying the single variable domain of step (B) such that it binds serum albumin at said pH of said intracellular compartment. Alternatively steps (A) and (B) can be reversed as follows: (A), selecting for ligands comprising a single variable domain which binds serum albumin at said natural serum pH, (B) from the ligands selected in (A), selecting ligands comprising a single variable domain which does not bind the regions of human serum albumin that bind FcRn, and (C) genetically modifying the single variable domain of step (B) such that it binds serum albumin at said pH of said intracellular compartment.

An assay to determine if a single variable domain does not competitively inhibit the binding of human serum albumin to FcRn: A competition BIAcore experiment can be used to determine if a single variable domain competitively inhibits the binding of serum albumin to a FcRn. One experimental protocol for such an example is as follows. After coating a CM5 sensor chip (Biacore AB) at 25° C. with approximately 1100 resonance units (RUs) of a purified FcRn at pH 7.4, human serum albumin (HSA), is injected over the antigen surface at a single concentration (e.g., 1 um) alone, and in combination with a dilution series of mixtures, each mixture having HSA and increasing amounts of the single variable domain in question. The resulting binding RUs are determined for the HSA alone and each of the HSA/single variable domain mixtures. By comparing the bound RUs of HSA alone with the bound RUs of HSA+single variable domain, one will be able to see whether the FcRn competes with the single variable domain to bind HSA. If it does compete, then as the single variable domain concentration in solution is increased, the RUs of HSA bound to FcRn will decrease. If there is no competition, then adding the single variable domain will have no impact on how much HSA binds to FcRn. This competition assay can optionally be repeated at pH 5.0 for a single variable domain which binds HAS at pH 5.0 in order to determine if the single variable domain competitively inhibits the binding of serum albumin to a FcRn at pH 5.0.

These ligands which have a single variable domain, which contains one single variable domain (monomer) or more than one single variable domains (e.g., multimer, fusion protein, conjugate, and dual specific ligand as defined herein) which specifically binds to serum albumin, where the single variable domain specifically binds serum albumin both at a natural serum pH, and at an intracellular vesicle pH, can further comprise at least one additional single variable domain, where each additional single variable domain specifically binds an antigen other than serum albumin at a natural serum pH, but does not bind the antigen at an intracellular vesicle pH. The intracellular vesicle pH can range from about 7.4 to 4.8. The natural serum pH is about 7.4, and the pH of said intracellular vesicle ranges from about 4.8 to 5.2, and in some embodiments, the pH of said intracellular vesicle is about 5.

A method based on the above ideas, includes the use of a bispecific binder with affinity for a serum albumin to prolong half-life and an affinity to a desired target antigen, as described above, to direct a bound antigen for degradation, or recycling. As described above, a serum albumin binding moiety is selected, such that binding is of high affinity at pH 5.0, such that the molecule would be sorted for non-degradation in the endosome by an FcRn mediated process. A desired target antigen binding moiety is then selected using a similar technique as described above, except that, instead of selecting for high affinity binding at pH 7.4 and pH 5, selection for high affinity binding at pH 7.4 is performed, and low or zero affinity for the target antigen at pH 5. One way to achieve this is by selecting for moieties with histidines in the contact surface. A fusion protein between the 2 molecules is then made by conventional molecular biology techniques, either by chemical derivitization and crosslinking, or by genetic fusion. The result is an increase in potency of a given AlbudAb™ (a dAb which specifically binds serum albumin) in vivo, by designing a SA binding dAb that binds SA at pH 5, while having a partner dAb that binds a ligand, which has low or zero affinity at pH 5. Though not wishing to be bound by theory, upon endosomal recycling, the target molecule will be released, and targeted to a degradative endosome and degraded, while the AlbudAb™ (a dAb which specifically binds serum albumin) is recycled to bind a fresh ligand via FcRn mediated recycling. This method offers a key advantage over PEGylated molecules or other half life extension technologies, where this pathway is not available for regeneration. Presumably in these cases, the bound ligand just sits on the PEGylated moiety and occupies it, whereas antibodies with intact Fc regions can be regenerated and recycled.

Described herein is a method of directing an antigen for degradation comprising administering a ligand which has at least one single variable domain, where the single variable domain specifically binds serum albumin both at a natural serum pH, and at an intracellular vesicle pH, and which further has at least one additional single variable domain, wherein the single variable domain specifically binds an antigen other than serum albumin at a natural serum pH, but does not bind said antigen at an intracellular vesicle pH, thus targeting the antigen other than serum albumin for degradation. Also described herein is, a ligand further comprising at least one additional single variable domain, wherein said single variable domain specifically binds an antigen other than serum albumin at a natural serum pH, but does not bind said antigen at an intracellular vesicle pH.

Selecting dAbs In Vitro in the Presence of Metabolites

Encompassed by the ligands described herein, is a ligand comprising a single variable domain, which contains one single variable domain (monomer) or more than one single variable domains (e.g., multimer, fusion protein, conjugate, and dual specific ligand as defined herein) which specifically binds to serum albumin, where the single variable domain specifically binds human serum albumin, and where specific binding of serum albumin by the single variable domain is not blocked by binding of drugs and/or metabolites and/or small molecules to one or more sites on serum albumin. The one or more sites on human serum albumin include Sudlow site 1 and Sudlow site 2. The one or more sites can be located on any combination of one or more domains of human serum albumin selected from the group consisting of domain I, domain II and domain III.

Encompassed by the ligands described herein, is a ligand comprising a single variable domain, which contains one single variable domain (monomer) or more than one single variable domains (e.g., multimer, fusion protein, conjugate, and dual specific ligand as defined herein) which specifically binds to serum albumin, where the single variable domain specifically binds human serum albumin, and where specific binding of serum albumin by said single variable domain does not alter the binding characteristics of serum albumin for drugs and/or metabolites and/or small molecule bound to SA. In one embodiment the single variable domain of the ligand binds serum albumin in both the presence and/or absence of a drug, metabolite or other small molecule. And in another embodiment, the specific binding of serum albumin by said single variable domain does not alter the binding characteristics of serum albumin for drugs and/or metabolites and/or small molecules bound to SA naturally in vivo, including, but preferably not limited to those drugs and/or metabolites and/or small molecules described in Fasano et al. (2005) 57(12):787-96. The extraordinary ligand binding properties of human serum albumin, and Bertucci, C. et al. (2002) 9(15):1463-81, Reversible and covalent binding of drugs to human serum albumin: methodological approaches and physiological relevance.

The drugs and/or metabolites and/or small molecules bound to SA may or may not overlap with the drugs and/or metabolites and/or small molecules which do not substantially inhibit or compete with serum albumin for binding to the single variable domain. The drugs and/or metabolites include, but are preferably not limited to warfarin, ibuprofen, vitamin B6, theta bilirubin, hemin, thyroxine, fatty acids, acetaldehyde, fatty acid metabolites, acyl glucuronide, metabolites of bilirubin, halothane, salicylate, benzodapenes and 1-O-gemfibrozil-B-D-glucuronide. This inhibition or competition with serum albumin for binding to the single variable domain by small molecules may occur by both direct displacement and by allosteric effects as described for small molecule binding induced changes on the binding of other small molecules, see Ascenzi et al. (2006) Mini Rev. Med. Chem. 6(4):483-9. Allosteric modulation of drug binding to human serum albumin, and Ghuman J. et al. (2005) J. Mol. Biol. 353(1):38-52 Structural basis of the drug-binding to human serum albumin. In one embodiment the small molecule, either alone, or in concert with one or more other small molecules, and/or metabolites, and/or proteins and/or drugs, binds serum albumin. In another embodiment, the small molecule either alone, or in concert with one or more other small molecules, and/or metabolites, and/or proteins and/or drugs, does not substantially inhibit or compete with serum albumin for binding to the single variable domain. In another embodiment, the small molecule, either alone or in concert with one or more other small molecules, and/or metabolites, and/or proteins and/or drugs, substantially inhibits or competes with serum albumin for binding to the single variable domain.

The single variable domain can be an antibody single variable domain. The antibody single variable domain can be a VH3 domain. The antibody single variable domain can be a V kappa domain. The single variable domain can comprise one or more non-immunoglobulin scaffolds. The non-immunoglobulin scaffold can include one or more of, but is preferably not limited to, CTLA-4, lipocallin, SpA, GroEL and fibronectin, and includes an Affibody™ and an Avimer™.

Described herein is a method of selecting a single variable domain which binds serum albumin, comprising selecting a first variable domain by its ability to bind to serum albumin in the presence of one or more metabolites and/or drugs, where the selection is performed in the presence of the one or more metabolites and/or drugs. Also described herein is a method for producing a dual specific ligand comprising a first immunoglobulin single variable domain having a first binding specificity for serum albumin in the presence of one or metabolite and/or drug, and a second immunoglobulin single variable domain having a second binding specificity, the method comprising the steps of: (a) selecting a first variable domain by its ability to bind to a first epitope in the presence of one or more metabolites and/or drugs, (b) selecting a second variable domain by its ability to bind to a second epitope, (c) combining the variable domains; and (d) selecting the ligand by its ability to bind to serum albumin in the presence of said one or more metabolites and/or ligands and said second epitopes. This method can also include a step where the first variable domain is selected for binding to said first epitope in absence of a complementary variable domain, and/or where the first variable domain is selected for binding to said first epitope in the presence of a third complementary variable domain in which said third variable domain is different from said second variable domain. These selection steps can be performed in the presence of a mixture of metabolites and/or drugs and/or proteins and/or small molecules. The selection steps can also be performed as follows: (a) selecting single variable domains which bind serum albumin in the presence of a first metabolite and/or drug and/or small molecule; and (b) from domains selected in step (a), a domain is selected in the presence of a second metabolite and/or drug and/or small molecule. Also encompassed is a method for producing a dual specific ligand having a first immunoglobulin single variable domain having a first binding specificity for serum albumin in the presence of one or metabolite and/or drug and/or small molecule, and a second immunoglobulin single variable domain having a second binding specificity, the method having the steps of: (a) selecting first variable domains by their ability to bind to serum albumin in the presence of one or more metabolites and/or drugs and/or small molecules, (b) selecting second variable domains by their ability to bind to an epitope, (c) combining the variable domains to provide ligands comprising a first and a second variable domain; and (d) from the ligands provided by step (c), and selecting a ligand by its ability to bind to serum albumin in the presence of the one or more metabolites and/or drugs and its ability to bind to said epitopes, thereby producing a dual specific ligand. In one embodiment, the first variable domain is selected for binding to serum albumin in absence of a complementary variable domain. In another embodiment, the first variable domain is selected for binding to the first epitope in the presence of a complementary variable domain in which the complementary variable domain is different from the second variable domain.

Linkers

Connecting an AlbudAb™ (a dAb which specifically binds serum albumin) (anti-serum albumin domain antibody or single variable domain) to another biologically active moiety can be obtained by recombinant engineering techniques. Basically, the genes encoding both proteins of interest are fused in frame. Several formats can be considered where the anti-serum albumin domain antibody is either at the N-terminal end of the fusion (i.e. AlbudAb™-Y where Y is a biologically active polypeptide), at the C-terminal end of the fusion (i.e. Z-AlbudAb™ where Z is a biologically active peptide). In some instances, one may consider fusing more than one biologically active polypeptide to an AlbudAb™ (a dAb which specifically binds serum albumin), resulting in a number of possibilities regarding the fusion design. For example, the fusion could be as follows: Z—Y-AlbudAb™, Z-AlbudAb™-Y or AlbudAb™-Z—Y.

In all these fusion molecules, two polypeptides are covalently linked together via at least one peptide bond. In its most simplistic approach, the AlbudAb™ (a dAb which specifically binds serum albumin) and the biologically polypeptide(s) are directly linked. Thus, the junction between the AlbudAb™ (a dAb which specifically binds serum albumin) and the polypeptide would be as follows:

a) For an AlbudAb™ (a dAb which specifically binds serum albumin)at the C-terminal end,

Where the AlbudAb™ is a VK:

xxxDIQ

xxxNIQ

xxxAIQ

xxxAIR

xxxVIW

xxxDIV

xxxDVV

xxxEIV

xxxETT

Where the AlbudAb™ (a dAb which specifically binds serum albumin) is a Vλ:

xxxQSV

xxxQSA

xxxSYE

xxxSSE

xxxSYV

xxxLPV

xxxQPV

xxxQLV

xxxQAV

xxxNFM

xxxQTV

xxxQAG

Where the AlbudAb™ (a dAb which specifically binds serum albumin) is a VH (e.g., human VH):

xxxQVQ

xxxQMQ

xxxEVQ

xxxQIT

xxxQVT

xxxQLQ

Where the AlbudAb™ (a dAb which specifically binds serum albumin) is a VHH (e.g., Camelid heavy chain variable domain):

xxxEVQ

xxxQVQ

xxxDVQ

xxxQVK

xxxAVQ

b) For an AlbudAb™ (a dAb which specifically binds serum albumin)at the N-terminal end,

Where the AlbudAb™ (a dAb which specifically binds serum albumin) is a VK:

KVEIKxxx

KLEIKxxx

KVDIKxxx

RLEIKxxx

EIKRxxx

Where the AlbudAb™ (a dAb which specifically binds serum albumin) is a Vλ:

KVDVLxxx

KLDVLxxx

QLDVLxxx

Where the AlbudAb™ (a dAb which specifically binds serum albumin)is a VH (e.g., human VH):

VTVSSxxx

Where the AlbudAb™ (a dAb which specifically binds serum albumin)is a VHH (e.g., Camelid heavy chain variable domain):

VTVSSxxx

‘xxx’ represents the first or last three amino acids of the (first) biologically active polypeptide fused to the AlbudAb™ (a dAb which specifically binds serum albumin).

However, there may be instances where the production of a recombinant fusion protein that recovers the functional activities of both polypeptides may be facilitated by connecting the encoding genes with a bridging DNA segment encoding a peptide linker that is spliced between the polypeptides connected in tandem. Optimal peptide linker length is usually devised empirically: it can be as short as one amino acid or extend up to 50 amino acids. Linkers of different designs have been proposed and are well know in the art. The following examples are meant to provide a broad—but not comprehensive—list of possible linker approaches:

1. Flexible Linkers:

Flexible linkers are designed to adopt no stable secondary structure when connecting two polypeptide moieties, thus allowing a range of conformations in the fusion protein. These linkers are preferably hydrophilic in nature to prevent these from interacting with one or both fused polypeptides. Usually small polar residues such as glycine and serine are prevalent in those linkers in order to increase the flexible and hydrophilic characteristics of the peptide backbone, respectively. The length of these linkers is variable and best determined either empirically or with the aid of 3D computing approaches. In general, a preferred linker length will be the smallest compatible with good expression, good solubility and full recovery of the native functions and structures of interest. Because of their flexible characteristics, flexible linkers may constitute good substrates for endogenous proteases. In general, unless it is a desirable feature flexible linkers are devoid of amino acids such as charged amino acids or large hydrophobic/aromatic which are readily recognized by endogenous proteases with broad substrate specificity. In addition cysteine residues are preferably avoided since free cysteines can react together to form cysteines, thereby resulting in (i) bridging two fusion proteins via the linkers, and/or (ii) compromised expression/folding of the fusion protein if one or more of the bioactive polypeptides comprises one or more cysteine residue (‘cysteine scrambling’).

Examples of flexible linkers are: (i) glycine-rich linkers based on the repetition of a (GGGGS)_{y} motif where y is at least 1, though y can be 2, 3, 4, 5, 6, 7, 8 and 9, or more (see PCT International Publications No: EP 0 753 551, U.S. Pat. No. 5,258,498, EP 0 623 679), (ii) serine-rich linkers based on the repetition of a (SSSSG)_{y} motif where y is at least 1, though y can be 2, 3, 4, 5, 6, 7, 8 and 9, or more (see PCT International Publications No: EP 0 573 551, U.S. Pat. No. 5,525,491).

2. Constrained Linkers:

Constrained linkers are designed to adopt a stable secondary structure when connecting two polypeptide moieties, thus restricting the range of conformations in the fusion protein. Such linkers usually adopt a helical structure spanning several turns.

Again the length of these linkers is variable and best determined either empirically or with the aid of computing approaches. The main reason for choosing constrained linkers is to maintain the longest distance between each polypeptide of the fusion. This is particularly relevant when both polypeptides have a tendency to form hetero-aggregates. By virtue of their structure, constrained linkers can also be more resistant to proteolytic degradation, thereby offering an advantage when injected in vivo.

Examples of constrained linkers are cited in PCT International Publications No: WO 00/24884 (e.g. SSSASASSA, GSPGSPG, or ATTTGSSPGPT), U.S. Pat. No. 6,132,992 (e.g. helical peptide linkers).

3. ‘Natural’ Linkers:

Natural linkers are polypeptide sequences (of variable lengths) that—by opposition—are not synthetic, i.e. the polypeptide sequences composing the linkers are found in nature. Natural linkers can be either flexible or constrained and can be very diverse in amino acid sequence and composition. Their degree of resistance to proteolysis depends on which proteins they originate from and which biological environment these proteins are facing in nature (extracellular, intracellular, prokaryotic, eukaryotic, etc). One class of linkers is particularly relevant for the development of biological therapeutics in man: linkers based on peptides found in human proteins. Indeed such linkers are by nature non- or very weakly—immunogenic and therefore potentially safer for human therapy.

Examples of natural linkers are: (i) KESGSVSSEQLAQFRSLD (see Bird et al. (1988) Science, 242, 423-426), (ii) sequences corresponding to the hinge domain of immunoglobulins devoid of light chains (see Hamers-Casterman et al. (1993) Nature, 363, 446-448 and PCT International Publication No: WO 096/34103). Examples of linkers for use with anti-albumin domain antibodies (e.g., human, humanized, camelized human or Camelid VHH domain antibodies) are EPKIPQPQPKPQPQPQPQPKPQPKPEPECTCPKCP and GTNEVCKCPKCP. Other linkers derived from human and camelid hinges are disclosed in EPO656946, incorporated herein by reference. The hinge derived linkers can have variable lengths, for example from 0 to about 50 amino acids, including 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids.

As used herein, “drug” refers to any compound (e.g., small organic molecule, nucleic acid, polypeptide) that can be administered to an individual to produce a beneficial, therapeutic or diagnostic effect through binding to and/or altering the function of a biological target molecule in the individual. The target molecule can be an endogenous target molecule encoded by the individual's genome (e.g. an enzyme, receptor, growth factor, cytokine encoded by the individual's genome) or an exogenous target molecule encoded by the genome of a pathogen (e.g. an enzyme encoded by the genome of a virus, bacterium, fungus, nematode or other pathogen).

The drug composition can be a conjugate wherein the drug is covalently or noncovalently bonded to the polypeptide binding moiety. The drug can be; covalently or noncovalently bonded to the polypeptide binding moiety directly or indirectly (e.g., through a suitable linker and/or noncovalent binding of complementary binding partners (e.g., biotin and avidin)). When complementary binding partners are employed, one of the binding partners can be covalently bonded to the drug directly or through a suitable linker moiety, and the complementary binding partner can be covalently bonded to the polypeptide binding moiety directly or through a suitable linker moiety. When the drug is a polypeptide or peptide, the drug composition can be a fusion protein, wherein the polypeptide or peptide, drug and the polypeptide binding moiety are discrete parts (moieties) of a continuous polypeptide chain. As described herein, the polypeptide binding moieties and polypeptide drug moieties can be directly bonded to each other through a peptide bond, or linked through a suitable amino acid, or peptide or polypeptide linker.

### Decreased Immunogenicity

Described herein is a method of reducing the immunogenicity of a pharmaceutical agent, comprising modifying said agent so that the agent further contains a single variable domain region, where the single variable domain specifically binds serum albumin in vivo and/or ex vivo, and where the agent can include a drug, a metabolite, a ligand, an antigen and a protein. The serum albumin can be human serum albumin. The single variable domain can be an immunoglobulin single variable domain. The immunoglobulin single variable domain can be a VH antibody single variable domain. The VH single variable domain can be a VH3 single variable domain. The VH3 single variable domain can be a human VH3 single variable domain. The single variable domain can be a Vkappa or a Vlambda antibody single variable domain. The antibody single variable domain can comprise a set of four Kabat framework regions (FRs which are encoded by VH3 framework germ line antibody gene segments. The VH3 framework is selected from the group consisting of DP47, DP38 and DP45. The antibody single variable domain can contain a set of four Kabat framework regions (FRs), which are encoded by VKappa framework germ line antibody gene segments. A nonlimiting example of a Kappa framework is DPK9. The single variable domain can contain an immunoglobulin or non-immunoglobulin scaffold which contains CDR1, CDR2 and/or CDR3 regions, wherein at least one of the CDR1, CDR2 and CDR3 regions is from an antibody variable domain which specifically binds serum albumin. The non-immunoglobulin scaffold can include, but is preferably not limited to, CTLA-4, lipocallin, SpA, Affibody™, GroEL, Avimers™ and fibronectin. The serum albumin can be human serum albumin. The immunoglobulin single variable domain and/or the non-immunoglobulin single variable domain can specifically bind to human serum albumin with a Kd of less than 300 nM. The immunoglobulin single variable domain and/or the non-immunoglobulin single variable domain can specifically bind to both human serum albumin and one or more non-human serum albumins, with Kd values within 10 fold of each other. The immunoglobulin single variable domain and/or non-immunoglobulin single variable domain can specifically bind to both human serum albumin and one or more non-human serum albumins, and wherein the T beta half life of the ligand is substantially the same as the T beta half life of human serum albumin in a human host. Further, the immunoglobulin single variable domain and/or non-immunoglobulin single variable domain can specifically bind to Domain II of human serum albumin. The immunoglobulin single variable domain and/or the non-immunoglobulin single variable domain can further specifically bind serum albumin both at a natural serum pH, and at an intracellular vesicle pH. The specific binding of serum albumin by said immunoglobulin single variable domain and/or the non-immunoglobulin single variable domain is preferably not substantially blocked by binding of drugs and/or metabolites to one or more sites on serum albumin. In one embodiment, the specific binding of serum albumin by the single variable domain does not alter the binding characteristics of serum albumin for drugs and/or metabolites and/or small molecules bound to SA. In one embodiment the method of modifying the agent results in the formation of an modified agent having a formula comprising: a-(X)n1-b-(Y)n2-c-(Z)n3-d or a-(Z)n3-b-(Y)n2-c-(X)n-d, wherein X is a polypeptide drug that has binding specificity for a first target; Y is a single variable domain, e.g. an antibody single variable domain that specifically binds serum albumin in vivo and/or ex vivo; Z is a polypeptide drug that has binding specificity for a second target; a, b, c and d are independently a polypeptide comprising one to about amino acid residues or absent; n1 is one to about 10; n2 is one to about 10; and n3 is zero to about 10. In a further embodiment, when n1 and n2 are both one and n3 is zero, X does not comprise an antibody chain or a fragment of an antibody chain.

The invention is further described, for the purposes of illustration only, in the following examples. As used herein, for the purposes of dAb nomenclature, human TNFα is referred to as TAR1 and human TNFα receptor 1 (p55 receptor) is referred to as TAR2.

### J. Treatment of Rheumatoid Arthritis

In a preferred embodiment, ligands as described herein can be used to treat rheumatoid arthritis.

In one aspect, the invention provides methods of treating rheumatoid arthritis, comprising the use of one or more single domain antibody polypeptide constructs, wherein one or more of the constructs antagonizes human TNFα's binding to a receptor. The present invention encompasses compositions comprising one or more single domain antibody polypeptide constructs that antagonize human TNFα's binding to a receptor, and dual specific ligands in which one specificity of the ligand is a single domain antibody directed toward TNFα and a second specificity is a single domain antibody directed to VEGF or HSA. The present invention further encompasses dual specific ligands in which one specificity of the ligand is directed toward VEGF and a second specificity is directed to HSA.

In one embodiment the invention provides methods of treatment of rheumatoid arthritis comprising administering a composition comprising one or more single domain antibody polypeptide constructs, wherein one or more of the constructs antagonizes human TNFα's binding to a receptor, and/or prevents an increase in arthritic score when administered to a mouse of the Tg197 transgenic mouse model of arthritis, and/or neutralizes TNF-α in the L929 cytotoxicity assay. In particular, methods of treatment of arthritis comprise the administration of a composition comprising one or more single domain antibody polypeptide constructs, wherein one or more of the constructs antagonizes human TNFα's binding to a receptor, and wherein the administration of the composition to a Tg197 transgenic mouse prevents an increase in arthritic score.

a) Receptor Binding Assays

Ligands for the treatment of rheumatoid arthritis can interfere with the binding of TNF-α to a TNF-α receptor. The receptor can be an isolated (usually membrane-bound) receptor, or it can be a receptor present on a cell, either in vitro or in vivo.

Assays for the measurement of TNF-α receptor binding and interference with such binding by ligands as described herein are described below in Example 6. These include ELISAs (Example 6, section 1.3.1), BIAcore analyses (Example 6, section 1.3.2) and biochemical receptor binding assays using both isolated (or membrane-associated) receptors (Example 6, section 1.3.3) and receptors expressed on the surface of cultured cells (Example 6, section 1.3.3).

As used herein, the term “antagonizes binding” of the receptor refers to the ability or effect of a given antibody polypeptide construct to interfere with the binding of TNF-α (or VEGF or other factor) to a cognate receptor. Antagonism is measured using one or more of the in vitro, cell-based or in vivo assays as described herein. Thus, the receptor can be isolated, membrane bound, or present on the cell surface. A construct interferes with or antagonizes binding to a cognate receptor (e.g., TNFR1, TNFR2, VEGFR1, VEGFR2) if there is a statistically significant decrease in binding detected in the presence of the construct relative to the absence of the construct. Alternatively, a construct interferes with binding if there is at least a 10% decrease in measured binding in the presence of the construct, relative to its absence.

b) L929 Cytotoxicity Assay

Ligands for the treatment of rheumatoid arthritis can interfere with the cytotoxic effects of TNF-α in the L929 cytotoxicity assay. This assay, based on the assay described by Evans et al., 2000, Molecular Biotechnology 15: 243-248, is described in Example 6, section 1.3.3. Anti-TNF-α ligands useful for the treatment of rheumatoid arthritis can neutralize the activity of TNF-α in this cell assay.

As used herein, the term “neutralizing,” when used in reference to an antibody or dAb polypeptide as described herein, means that the polypeptide interferes with a measurable activity or function of the target antigen. A polypeptide is a “neutralizing” polypeptide if it reduces a measurable activity or function of the target antigen by at least 50%, and preferably at least 60%, 70%, 80%, 90%, 95% or more, up to and including 100% inhibition (i.e., no detectable effect or function of the target antigen). Thus, where the target is TNF-α, neutralizing activity can be assessed using the standard L929 cell killing assay described herein or by measuring the ability of an anti-TNF-α polypeptide construct to inhibit TNF-α-induced expression of ELAM-1 on HUVEC, which measures TNF-α-induced cellular activation.

Additional assays for antibody polypeptide interference with the receptor biding activity of TNF-α include the HeLa IL-8 assay also described in Example 6, section 1.3.3.

c) In Vivo Assays.

The efficacy of anti-TNF-α ligands as described herein can be assessed using the Tg197 transgenic mouse arthritis model. Tg197 mice are transgenic for the human TNF-globin hybrid gene and heterozygotes at 4-7 weeks of age develop a chronic, progressive polyarthritis with histological features in common with rheumatoid arthritis (Keffer et al., 1991, EMBO J. 10: 4025-4031). The arthritic phenotype can be scored by assessing joint mobility and joint swelling. The arthritic phenotype of the joints can be scored by X-ray imaging of the joints and by histolopathological analysis of fixed sections of the knee and ankle/paw joints.

Experimental treatment to assess the efficacy of a given antibody polypeptide construct is performed as follows.

1) To test the prevention of arthritis with an antibody polypeptide construct, animals are treated as follows:

a) heterozygous Tg197 mice are divided into groups of 10 animals with equal numbers of males and females. Treatment commences at 3 weeks of age, with weekly intraperitoneal administration of the antibody polypeptide in PBS, or PBS alone in the control animals;

b) weigh the mice weekly;

c) score the mice for macrophenotypic signs of arthritis according to the following system: 0=no arthritis (normal appearance and flexion), 1=mild arthritis (joint distortion), 2=moderate arthritis (swelling, joint deformation), 3=heavy arthritis (severely impaired movement).

The studies should best be performed such that the individual scoring is blinded to the test groupings. The preferred mechanism of antibody delivery for this assay is IP injection. However, the assay can be adapted to use subcutaneous injection, IV injection (e.g., via tail vein), intramuscular injection, or oral, inhalation or topical administration.

A treatment is effective in the Tg197 model system if the average arthritic score in the treatment group is lower (by a statistically significant amount) than that of the vehicle-only control group. Treatment is also considered effective if the average arthritic score is lower by at least 0.5 units, at least 1.0 units, at least 1.5 units or by at least 2 units relative to the vehicle-only control animals. Alternatively, the treatment is effective is the average arthritic score remains at or is lowered to 0 to 0.25 throughout the course of the therapeutic regimen.

A treatment is effective in the Tg197 model system if the average arthritic score in the treatment group increases during the course of the experiment but the start of this increase is delayed when compared with the vehicle only control. Treatment is also considered effective if the start of the increase in the average arthritic score of the treatment group when compared to the vehicle only control is delayed by 0.5 weeks, at least 1 week, at least 1.5 weeks, at least 2 weeks or by greater than 3 weeks.

As an alternative to the macrophenotypic scoring, at various intervals during treatment, ankle/paw and knee joints can be fixed and analyzed histopathologically using the following system: 0=no detectable pathology; 1=hyperplasia of the synovial membrane and presence of polymorphonuclear infiltrates; 2=pannus and fibrous tissue formation and focal subchondral bone erosion; 4=extensive articular cartilage destruction and bone erosion. Treatment is considered effective if the average histopathological score is lower (by a statistically significant amount) than that of the vehicle control group. Treatment is also considered effective if the average histopathological score is lower by at least 0.5 units, at least 1.0 unit, at least 1.5 units, at least 2.0 units, at least 2.5 units, at least 3.0 units, or by at least 3.5 units relative to the vehicle-only control group. Alternatively, the treatment is effective is the average histopatholigical score remains at or is lowered to 0 to 0.5 throughout the course of the therapeutic regimen.

2) To test the effect of an antibody polypeptide construct (anti-TNF-α, anti-VEGF, etc.) on established arthritis, the assay can be performed on Tg197 animals as described above, only beginning treatment at 6 weeks of age, a time at which the animals have significant arthritic phenotypes. Scoring and efficacy analyses are also as described above. Anti-TNF-α dAb constructs as described herein can halt or reverse the progression of established arthritis in one or more of the model systems described.

In either format, treatment approaches include anti-TNF-α (e.g., anti-TNF-α dAb as described herein) in monomeric, dimeric or other multimeric forms, anti-VEGF (e.g., anti-VEGF dAb as described herein, including also camelid anti-VEGF dAbs) in monomeric, dimeric or other multimeric forms, a dual specific format of anti-TNF/anti-VEGF, and individual or dual specific constructs bearing anti-HSA, PEG or other half-life modifying moiet(ies). Additionally, anti-VEGF compositions described herein can be administered in combination with other anti-TNF compositions, such as etanercept (Enbrel), D2E7 (Humira) and infliximab (Remicade). The effectiveness of such combination therapy can be assessed using, for example, the cell culture and in vivo model systems described herein.

Additional accepted animal models of arthritis include collagen induced arthritis (CIA), described, for example, by Horsfall et al., 1997, J. of Immunol. 159:5687), and pristane-induced arthritis, described, for example, by Stasluk et al., 1997, Immunol. 90:81.

Assays for anti-VEGF polypeptide construct effectiveness:

a) VEGF Receptor 2 Binding Assay

This method describes a VEGF receptor binding assay for measuring the ability of soluble domain antibodies (dAbs) to prevent VEGF₁₆₅ binding to VEGF Receptor 2.

VEGF is a specific mitogen for endothelial cells in vitro and a potent angiogenic factor in vivo, with high levels of the protein being expressed in various types of tumours. It is a 45 kDa glycoprotein that is active as a homodimer. So far five different isoforms have been described which occur through alternative mRNA splicing. Of these isoforms VEGF₁₂₁ and VEGF₁₆₅ are the most abundant.

The specific action of VEGF on endothelial cells is mainly regulated by two types of receptor tyrosine kinases (RTK), VEGF R1 (Flt-1), and VEGF R2 (KDR/Flk-1). However, it appears that the VEGF activities such as mitogenicity, chemotaxis, and induction of morphological changes are mediated by VEGF R2, even though both receptors undergo phosphorylation upon binding of VEGF.

A recombinant human VEGF R2/Fc chimera is used in this assay, comprising the extracellular domain of human VEGF R2 fused to the Fc region of human IgG₁. Briefly, the receptor is captured on an ELISA plate, then the plate is blocked to prevent non specific binding. A mixture of VEGF₁₆₅ and dAb protein is then added, the plate is washed and receptor bound VEGF₁₆₅ detected using a biotinylated anti-VEGF antibody and an HRP conjugated anti-biotin antibody. The plate is developed using a colorimetric substrate and the OD read at 450 nm. If the dAb blocks VEGF binding to the receptor then no colour is detected.

The assay is performed as follows. A 96 well Nunc Maxisorp assay plate is coated overnight at 4 C with 100μl per well of recombinant human VEGF R2/Fc (R&D Systems, Cat. No: 357-KD-050)@0.5 μg/ml in carbonate buffer. Wells are washed 3 times with 0.05% tween/PBS and 3 times with PBS. 200 μl per well of 2% BSA in PBS is added to block the plate and the plate is incubated for a minimum of 1 h at room temperature.

Wells are washed (as above), then 50 μl per well of purified dAb protein is added to each well. 50 μl of VEGF, @6 ng/ml in diluent (for a final concentration of 3 ng/ml), is then added to each well and the plate incubated for 2 hr at room temperature (for assay of supernatants; add 80 μl of supernatant to each well then 20 μl of VEGF@15 ng/ml).

The following controls should be included: 0 ng/ml VEGF (diluent only); 3 ng/ml VEGF (R&D Systems, Cat No: 293-VE-050); 3 ng/ml VEGF with 0.1 μg/ml anti-VEGF neutralizing antibody (R&D Systems cat #MAB293).

The plate is washed (as above) and then 100 μl biotinylated anti-VEGF antibody (R&D Systems, Cat No: BAF293), 0.5 μg/ml in diluent, is added and incubated for 2 hr at room temperature.

Wells are washed (as above) then add 100 μl HRP conjugated anti-biotin antibody (1:5000 dilution in diluent; Stratech, Cat No: 200-032-096). The plate is then incubated for 1 hr at room temperature.

The plate is washed (as above) ensuring any traces of Tween-20 have been removed to limit background in the subsequent peroxidase assay and to help the prevention of bubbles in the assay plate wells that will give inaccurate OD readings.

100 μl of SureBlue 1-Component TMB MicroWell Peroxidase solution is added to each well, and the plate is left at room temperature for up to 20 min. A deep blue soluble product will develop as bound HRP labelled conjugate reacts with the substrate. The reaction is stopped by the addition of 100 μl 1M hydrochloric acid (the blue colour will turn yellow). The OD, at 450 nm, of the plate should be read in a 96-well plate reader within 30 min of acid addition. The OD450 nm is proportional to the amount of bound streptavidin-HRP conjugate.

Expected result from the controls are as follows: 0 ng/ml VEGF should give a low signal of <0.15 OD; 3 ng/ml VEGF should give a signal of >0.5 OD; and 3 ng/ml VEGF pre-incubated with 0.1 μg/ml neutralising antibody should give a signal<0.2 OD.

b) VEGF Receptor 1 Binding Assay

This assay measures the binding of VEGF₁₆₅ to VEGF R1 and the ability of dAbs to block this interaction.

A recombinant human VEGF R1/Fc chimera is used here, comprising the extracellular domain of human VEGF R1 fused to the Fc region of human IgG₁. The receptor is captured on an ELISA plate then the plate is blocked to prevent non specific binding. A mixture of _{VEGF}₁₆₅ and dAb protein is then added, the plate is washed and receptor bound VEGF₁₆₅ detected using a biotinylated anti-VEGF antibody and an HRP conjugated anti-biotin antibody. The plate is developed using a colorimetric substrate and the OD read at 450 nm. If the dAb blocks VEGF binding to the receptor then no colour will show.

The assay is performed as follows. A 96 well Nunc Maxisorp assay plate is coated overnight at 4 C with 100 μl per well of recombinant human VEGF R1/Fc (R&D Systems, Cat No: 321-FL-050)@0.1 μg/ml in carbonate buffer. Wells are washed 3 times with 0.05% tween/PBS and 3 times with PBS.

200 μl per well of 2% BSA in PBS is added to block the plate and the plate is incubated for a minimum of 1h at room temperature.

Wells are washed (as above), then 50 μl per well of purified dAb protein is added to each well. 50 μl of VEGF, @1 ng/ml in diluent (for a final concentration of 500 pg/ml), is then added to each well and the plate incubated for 1 hr at room temperature (assay of supernatants; add 80 μl of supernatant to each well then 20 μl of VEGF@2.5 ng/ml).

The following controls should be included: 0 ng/ml VEGF (diluent only); 500 pg/ml VEGF; and 500 pg/ml VEGF with 1 μg/ml anti-VEGF antibody (R&D Systems cat #MAB293).

The plate is washed (as above) and then 100 μl biotinylated anti-VEGF antibody, 50 ng/ml in diluent, is added and incubated for 1 hr at room temperature.

Wells are washed (as above) then add 100 μl HRP conjugated anti-biotin antibody (1:5000 dilution in diluent). The plate is then incubated for 1 hr at room temperature.

The plate is washed (as above), ensuring any traces of Tween-20 have been removed to limit background in the subsequent peroxidase assay and to help the prevention of bubbles in the assay plate wells that will give inaccurate OD readings.

100 μl of SureBlue 1-Component TMB MicroWell Peroxidase solution is added to each well, and the plate is left at room temperature for up to 20 min. A deep blue soluble product will develop as bound HRP labelled conjugate reacts with the substrate. The reaction is stopped by the addition of 100 μl 1M hydrochloric acid (the blue colour will turn yellow). The OD, at 450 nm, of the plate should be read in a 96-well plate reader within 30 min of acid addition. The OD450 nm is proportional to the amount of bound streptavidin-HRP conjugate.

Expected result from the controls: 0 ng/ml VEGF should give a low signal of <0.15 OD; 500 pg/ml VEGF should give a signal of >0.8 OD; and 500 pg/ml VEGF pre-incubated with 1 μg/ml neutralising antibody should give a signal<0.3 OD

c) Cell-Based Assay for VEGF Activity:

This bioassay measures the ability of antibody polypeptides (e.g., dAbs) and other inhibitors to neutralise the VEGF induced proliferation of HUVE cells. HUVE cells plated in 96 well plates are incubated for 72 hours with pre-equilibrated VEGF and dAb protein. Cell number is then measured using a cell viability dye.

The assay is performed as follows. HUVE cells are trypsinized from a sub-confluent 175 cm² flask. Medium is aspirated off, the cells are washed with 5 ml trypsin and then incubated with 2 ml trypsin at room temperature for 5 min. The cells are gently dislodged from the base of the flask by knocking against your hand. 8 ml of induction medium are then added to the flask, pipetting the cells to disperse any clumps. Viable cells are counted using trypan blue stain.

Cells are spun down and washed 2× in induction medium, spinning cells down and aspirating the medium after each wash. After the final aspiration the cells are diluted to 10⁵ cells/ml (in induction medium) and plated at 100 μl per well into a 96 well plate (10,000 cells/well). The plate is incubated for >2 h@37 C to allow attachment of cells.

60 μl dAb protein and 60 μl induction media containing 40 ng/ml VEGF₁₆₅ (for a final concentration of 10 ng/ml) is added to a v bottom 96 well plate and sealed with film. The dAb/VEGF mixture is then incubated at 37 C for 0.5-1 hour.

The dAb/VEGF plate is removed from the incubator and 100 μl of solution added to each well of the HUVEC containing plate (final volume of 200 μl). This plate is then returned to the 37 C incubator for a period of at least 72 hours.

Control wells include the following: wells containing cells, but no VEGF; wells containing cells, a positive control neutralising anti-VEGF antibody and VEGF; and control wells containing cells and VEGF only.

Cell viability is assessed by adding 20 μl per well Celltiter96 reagent, and the plate incubated at 37 C for 2-4 h until a brown colour develops. The reaction is stopped by the addition of 20 μl per well of 10% (w/v) SDS. The absorbance is then read at 490 nm using a Wallac microplate reader.

The absorbance of the no VEGF control wells is subtracted from all other values. Absorbance is proportional to cell number. The control wells containing control anti-VEGF antibodies should also exhibit minimum cell proliferation. The wells containing VEGF only should exhibit maximum cell proliferation.

d) In Vivo Assay for VEGF Activity:

The efficacy of anti-VEGF polypeptide constructs (monomers, multimers or dual- or multi-specific) can also be tested in the Tg197 transgenic mouse model of arthritic disease. Dosing regimens and scoring are essentially as described for anti-TNF-α polypeptide constructs.

4. Treatment of Crohn's Disease

Anti-TNF-α polypeptides as described herein can be used to treat Crohn's disease in humans. In one embodiment the invention provides methods of treatment of Crohn's disease or other inflammatory bowel disease (IBD) in which TNF-α is involved. The methods comprise administering a composition comprising one or more single domain antibody polypeptide constructs, wherein one or more of the constructs antagonizes human TNFα's binding to a receptor, and/or prevents an increase in acute or chronic inflammatory bowel score when administered to a mouse of the Tnf^{ΔARE} transgenic mouse model of IBD, and/or neutralizes TNF-α in the L929 cytotoxicity assay. In particular, methods of treatment of Crohn's or other inflammatory bowel disorders comprise the administration of a composition comprising one or more single domain antibody polypeptide constructs, wherein one or more of the constructs antagonizes human TNFα's binding to a receptor, and wherein the administration of the composition to a Tnf^{ΔARE} transgenic mouse prevents an increase or effects a decrease in acute or chronic inflammatory bowel score.

The Tnf^{ΔARE} transgenic mouse model of Crohn's disease was originally described by Kontoyiannis et al., 1999, Immunity 10: 387-398; see also Kontoyiannis et al., 2002, J. Exp. Med. 196: 1563-1574. These mice bear a targeted deletion mutation in the 3′ AU-rich elements (AREs) of TNF-α mRNA. AU-rich elements are involved in maintaining low mRNA stability, and their disruption leads to overexpression of murine TNF-α in these animals. The animals develop an IBD phenotype with remarkable similarity to Crohn's disease starting between 4 and 8 weeks of age. The basic histopathological characteristics include villus blunting and submucosal inflammation with prevailing PMN/macrophage and lymphocytic exudates, proceeding to patchy transmural inflammation and the appearance of lymphoid aggregates and rudimentary granulomata (Kontoyiannis et al., 2002, supra.). These animals also develop an arthritic phenotype and can thus also be used to separately evaluate the efficacy of anti-TNF-α treatments in RA.

Where treatment is to be evaluated for its effect in preventing IBD, treatment is initiated at, for example, 3 weeks of age, with initial weekly IP doses of a given antibody polypeptide construct. More or less frequent dosing intervals can be selected by one of skill in the art, depending upon the outcome of initial studies. Animals can then be monitored for bowel disease according to a standard scale as described in Kontoyiannis et al., 2002, supra. Paraffin-embedded intestinal tissue sections of ileum are histologically evaluated in a blinded fashion according to the following scale: Acute and chronic inflammation are assessed separately in a minimum of 8 high power fields (hpf) as follows—acute inflammatory score 0=0-1 polymorphonuclear (PMN) cells per hpf (PMN/hpf); 1=2-10 PMN/hpf within mucosa; 2=11-20 PMN/hpf within mucosa; 3=21-30 PMN/hpf within mucosa or 11-20 PMN/hpf with extension below muscularis mucosae; and 4=>30 PMN/hpf within mucosa or >20 PMN/hpf with extension below muscularis mucosae. Chronic inflammatory score 0=0-10 mononuclear leukocytes (ML) per hpf (ML/hpf) within mucosa; 1=11-20 ML/hpf within mucosa; 2=21-30 ML/hpf within mucosa or 11-20 ML/hpf with extension below muscularis mucosae; 3=31-40 ML/hpf within mucosa or 21-30 ML/hpf with extension below muscularis mucosae or follicular hyperplasia; and 4=>40 ML/hpf within mucosa or >30 ML/hpf with extension below muscularis mucosae or follicular hyperplasia. Total disease score per mouse is calculated by summation of the acute inflammatory or chronic inflammatory scores for each mouse.

To evaluate the effect of treatment on established disease, treatment can be begun at 6-8 weeks of age, with scoring performed in the same manner.

Treatment is considered effective if the average histopathological disease score is lower in treated animals (by a statistically significant amount) than that of the vehicle control group. Treatment is also considered effective if the average histopathological score is lower by at least 0.5 units, at least 1.0 units, at least 1.5 units, at least 2.0 units, at least 2.5 units, at least 3.0 units, or by at least 3.5 units relative to the vehicle-only control group. Alternatively, the treatment is effective if the average histopatholigical score remains at or is lowered to 0 to 0.5 throughout the course of the therapeutic regimen.

Other models of IBD include, for example, the DSS (dextran sodium sulfate) model of chronic colitis in BALB/c mice. The DSS model was originally described by Okayasu et al., 1990, Gastroenterology 98: 694-702 and was modified by Kojouharoff et al., 1997, Clin Exp. Immunol. 107: 353-358 (see also WO 2004/041862, which designates the U.S., incorporated herein by reference). BALB/c mice weighing 21-22 g are treated to induce chronic colitis by the administration of DSS in their drinking water at 5% w/v in cycles of 7 days of treatment and 12 days recovery interval without DSS. The 4^{th} recovery period can be extended from 12 to 21 days to represent a chronic inflammation status, rather than the acute status modeled by shorter recovery. After the last recovery period, treatment with antibody polypeptide, e.g., anti-TNF-α polypeptide as described herein is administered. Weekly administration is recommended initially, but can be adjusted by one of skill in the art as necessary (especially, e.g., to evaluate dosage forms with different half-life modifying moieties). At intervals during treatment, animals are killed, intestine is dissected and histopathological scores are assessed as described herein or as described in Kojouharoff et al., 1997, supra.

Other animal models of inflammatory bowel disease include the chronic intestinal inflammation induced by rectal instillation of 2,4,6-Trinitrobenzene sulfonic acid (TNBS; method described by Neurath et al., 1995, J. Exp. Med. 182: 1281; see also U.S. Pat. No. 6,764,838, incorporated herein by reference). Histopathological scoring can be performed using the same standard described above.

Comparison with other anti-TNF-α agents:

Disclosed herein are anti-TNF-α dAb constructs effective for the treatment of RA, Crohn's disease and other TNF-α mediated disorders. In one aspect, the effectiveness of the anti-TNF-α dAb constructs is greater than or equal to that of an agent selected from the group consisting of etanercept (ENBREL), infliximab (REMICADE) and D2E7 (HUMIRA; see U.S. Pat. No. 6,090,382, incorporated herein by reference).

Clinical trials of a recombinant version of the soluble human TNFR (p75) linked to the Fc portion of human IgG1 (sTNFR(p75):Fc, ENBREL, Immunex) have shown that its administration resulted in significant and rapid reductions in RA disease activity (Moreland et al., 1997, N. Eng. J. Med., 337:141-147). In addition, preliminary safety data from a pediatric clinical trial for sTNFR(p75):Fc indicates that this drug is generally well-tolerated by patients with juvenile rheumatoid arthritis (JRA) (Garrison et al, 1998, Am. College of Rheumatology meeting, Nov. 9, 1998, abstract 584).

As noted above, ENBREL is a dimeric fusion protein consisting of the extracellular ligand-binding portion of the human 75 kilodalton (p75) TNFR (GenBank Accession No. P20333) linked to the Fc portion of human IgG1. The Fc component of ENBREL contains the CH2 domain, the CH3 domain and hinge region, but not the CH1 domain of IgG 1. ENBREL is produced in a Chinese hamster ovary (CHO) mammalian cell expression system. It consists of 934 amino acids and has an apparent molecular weight of approximately 150 kilodaltons (Smith et al., 1990, Science 248:1019-1023; Mohler et al., 1993, J. Immunol. 151:1548-1561; U.S. Pat. No. 5,395,760 (Immunex Corporation, Seattle, Wash.; incorporated herein by reference); U.S. Pat. No. 5,605,690 (Immunex Corporation, Seattle, Wash.; incorporated herein by reference).

A monoclonal antibody directed against TNF-α. (infliximab, REMICADE, Centocor), administered with and without methotrexate, has demonstrated clinical efficacy in the treatment of RA (Elliott et al., 1993, Arthritis Rheum. 36:1681-1690; Elliott et al., 1994, Lancet 344:1105-1110). These data demonstrate significant reductions in Paulus 20% and 50% criteria at 4, 12 and 26 weeks. This treatment is administered intravenously and the anti-TNF monoclonal antibody disappears from circulation over a period of two months. The duration of efficacy appears to decrease with repeated doses. The patient can generate antibodies against the anti-TNF antibodies which limit the effectiveness and duration of this therapy (Kavanaugh et al., 1998, Rheum. Dis. Clin. North Am. 24:593-614). Administration of methotrexate in combination with infliximab helps prevent the development of anti-infliximab antibodies (Maini et al., 1998, Arthritis Rheum. 41:1552-1563). Infliximab has also demonstrated clinical efficacy in the treatment of the inflammatory bowel disorder Crohn's disease (Baert et al., 1999, Gastroenterology 116:22-28).

As discussed in the background section, infliximab is a chimeric monoclonal IgG antibody bearing human IgG4 constant and mouse variable regions. The infliximab polypeptide is described in U.S. Pat. Nos. 5,698,195 and 5,656,272, which are incorporated herein by reference.

To compare efficacy with these or other anti-TNF-α compositions, one need only perform one or more of the receptor binding, cell-based or in vivo assays as described herein above using the anti-TNF-α dAb construct in parallel with the existing composition. This approach thus identifies those anti-TNF-α dAb constructs that show an effectiveness at inhibiting the effects of TNF-α in one or more of the assays that is equal to or greater than (in a statistically significant manner) the effectiveness of the comparison composition. Examples of such constructs and the analyses demonstrating equal or superior effectiveness are provided in the Examples.

### Example 1

### Selection of a Dual Specific scFv Antibody (K8) Directed Against Human Serum Albumin (HSA) and β-Galactosidase (β-Gal)

This example explains a method for making a dual specific antibody directed against β-gal and HSA in which a repertoire of V_{κ} variable domains linked to a germline (dummy) V_{H} domain is selected for binding to β-gal and a repertoire of V_{H} variable domains linked to a germline (dummy) V_{κ} domain is selected for binding to HSA. The selected variable V_{H} HSA and V_{κ} β-gal domains are then combined and the antibodies selected for binding to β-gal and HSA. HSA is a half-life increasing protein found in human blood.

Four human phage antibody libraries were used in this experiment.

| | | |
|---|---|---|
| Library 1 | Germline V_{κ}/DVT V_{H} | 8.46 × 10⁷ |
| Library 2 | Germline V_{κ}/NNK V_{H} | 9.64 × 10⁷ |
| Library 3 | Germline V_{H}/DVT V_{κ} | 1.47 × 10⁸ |
| Library 4 | Germline V_{H}/NNK V_{κ} | 1.45 × 10⁸ |

All libraries are based on a single human framework for V_{H} (V3-23/DP47 and J_{H}4b) and V_{κ} (O12/O2/DPK9 and J_{κ} 1) with side chain diversity incorporated in complementarity determining regions (CDR2 and CDR3).

Library 1 and Library 2 contain a dummy V_{κ} sequence, whereas the sequence of V_{H} is diversified at positions H50, H52, H52a, H53, H55, H56, H58, H95, H96, H97 and H98 (DVT or NNK encoded, respectively) (FIG. 1). Library 3 and Library 4 contain a dummy V_{H} sequence, whereas the sequence of V_{κ} is diversified at positions L50, L53, L91, L92, L93, L94 and L96 (DVT or NNK encoded, respectively) (FIG. 1). The libraries are in phagemid pIT2/ScFv format (FIG. 2) and have been preselected for binding to generic ligands, Protein A and Protein L, so that the majority of clones in the unselected libraries are functional. The sizes of the libraries shown above correspond to the sizes after preselection. Library 1 and Library 2 were mixed prior to selections on antigen to yield a single V_{H}/dummy V_{κ} library and Library 3 and Library 4 were mixed to form a single V_{κ}/dummy V_{H} library.

Three rounds of selections were performed on β-gal using V_{κ}/dummy V_{H} library and three rounds of selections were performed on HSA using V_{H}/dummy V_{κ} library. In the case of β-gal the phage titres went up from 1.1×10⁶ in the first round to 2.0×10⁸ in the third round. In the case of HSA the phage titres went up from 2×10⁴ in the first round to 1.4×10⁹ in the third round. The selections were performed as described by Griffith et al., (1993), except that KM13 helper phage (which contains a pIII protein with a protease cleavage site between the D2 and D3 domains) was used and phage were eluted with 1 mg/ml trypsin in PBS. The addition of trypsin cleaves the pIII proteins derived from the helper phage (but not those from the phagemid) and elutes bound scFv-phage fusions by cleavage in the c-myc tag (FIG. 2), thereby providing a further enrichment for phages expressing functional scFvs and a corresponding reduction in background (Kristensen & Winter, Folding & Design 3: 321-328, Jul. 9, 1998). Selections were performed using immunotubes coated with either HSA or β-gal at 100 μg/ml concentration.

To check for binding, 24 colonies from the third round of each selection were screened by monoclonal phage ELISA. Phage particles were produced as described by Harrison et al., Methods Enzymol. 1996;267:83-109. 96-well ELISA plates were coated with 100 μl of HSA or β-gal at 10 μg/ml concentration in PBS overnight at 4° C. A standard ELISA protocol was followed (Hoogenboom et al., 1991) using detection of bound phage with anti-M13-HRP conjugate. A selection of clones gave ELISA signals of greater than 1.0 with 50 μl supernatant.

Next, DNA preps were made from V_{H}/dummy V_{κ} library selected on HSA and from V_{κ}/dummy V_{H} library selected on β-gal using the QIAprep Spin Miniprep kit (Qiagen). To access most of the diversity, DNA preps were made from each of the three rounds of selections and then pulled together for each of the antigens. DNA preps were then digested with SalI/NotI overnight at 37° C. Following gel purification of the fragments, V_{κ} chains from the V_{κ}/dummy V_{H} library selected on β-gal were ligated in place of a dummy V_{κ} chain of the V_{H}/dummy V_{κ} library selected on HSA creating a library of 3.3×10⁹ clones.

This library was then either selected on HSA (first round) and β-gal (second round), HSA/β-gal selection, or on β-gal (first round) and HSA (second round), β-gal/HSA selection. Selections were performed as described above. In each case after the second round 48 clones were tested for binding to HSA and β-gal by the monoclonal phage ELISA (as described above) and by ELISA of the soluble scFv fragments. Soluble antibody fragments were produced as described by Harrison et al., (1996), and standard ELISA protocol was followed Hoogenboom et al. (1991) Nucleic Acids Res., 19: 4133, except that 2% Tween/PBS was used as a blocking buffer and bound scFvs were detected with Protein L-HRP. Three clones (E4, E5 and E8) from the HSA/β-gal selection and two clones (K8 and K10) from the β-gal/HSA selection were able to bind both antigens. scFvs from these clones were PCR amplified and sequenced as described by Ignatovich et al., (1999) J Mol Biol 1999 Nov. 26; 294(2):457-65, using the primers LMB3 and pHENseq. Sequence analysis revealed that all clones were identical. Therefore, only one clone encoding a dual specific antibody (K8) was chosen for further work (FIG. 3).

### Example 2

### Characterisation of the Binding Properties of the K8 Antibody

Firstly, the binding properties of the K8 antibody were characterised by the monoclonal phage ELISA. A 96-well plate was coated with 100 μl of HSA and β-gal alongside with alkaline phosphatase (APS), bovine serum albumin (BSA), peanut agglutinin, lysozyme and cytochrome c (to check for cross-reactivity) at 10 μg/ml concentration in PBS overnight at 4° C. The phagemid from K8 clone was rescued with KM13 as described by Harrison et al., (1996) and the supernatant (50 μl) containing phage assayed directly. A standard ELISA protocol was followed (Hoogenboom et al., 1991) using detection of bound phage with anti-M13-HRP conjugate. The dual specific K8 antibody was found to bind to HSA and β-gal when displayed on the surface of the phage with absorbance signals greater than 1.0 (FIG. 4). Strong binding to BSA was also observed (FIG. 4). Since HSA and BSA are 76% homologous on the amino acid level, it is not surprising that K8 antibody recognised both of these structurally related proteins. No cross-reactivity with other proteins was detected (FIG. 4).

Secondly, the binding properties of the K8 antibody were tested in a soluble scFv ELISA. Production of the soluble scFv fragment was induced by IPTG as described by Harrison et al., (1996). To determine the expression levels of K8 scFv, the soluble antibody fragments were purified from the supernatant of 50 ml inductions using Protein A-Sepharose columns as described by Harlow and Lane, Antibodies: a Laboratory Manual, (1988) Cold Spring Harbor. OD₂₈₀ was then measured and the protein concentration calculated as described by Sambrook et al., (1989). K8 scFv was produced in supernatant at 19 mg/l.

A soluble scFv ELISA was then performed using known concentrations of the K8 antibody fragment. A 96-well plate was coated with 100 μl of HSA, BSA and β-gal at 10 μg/ml and 100 μl of Protein A at 1 μg/ml concentration. 50 μl of the serial dilutions of the K8 scFv was applied and the bound antibody fragments were detected with Protein L-HRP. ELISA results confirmed the dual specific nature of the K8 antibody (FIG. 5).

To confirm that binding to β-gal is determined by the V_{κ} domain and binding to HSA/BSA by the V_{H} domain of the K8 scFv antibody, the V_{κ} domain was cut out from K8 scFv DNA by SalI/NotI digestion and ligated into a SalI/NotI digested pIT2 vector containing dummy V_{H} chain (FIGS. 1 and 2). Binding characteristics of the resulting clone K8V_{κ}/dummy V_{H} were analysed by soluble scFv ELISA. Production of the soluble scFv fragments was induced by IPTG as described by Harrison et al., (1996) and the supernatant (50μ) containing scFvs assayed directly. Soluble scFv ELISA was performed as described in Example 1 and the bound scFvs were detected with Protein L-HRP. The ELISA results revealed that this clone was still able to bind β-gal, whereas binding to BSA was abolished (FIG. 6).

### Example 3

### Selection of Single V_{H} Domain Antibodies Antigens A and B and Single V_{κ} Domain Antibodies Directed Against Antigens C and D

This example describes a method for making single V_{H} domain antibodies directed against antigens A and B and single V_{κ} domain antibodies directed against antigens C and D by selecting repertoires of virgin single antibody variable domains for binding to these antigens in the absence of the complementary variable domains.

Selections and characterisation of the binding clones is performed as described previously (see Example 5, PCT/GB 02/003014). Four clones are chosen for further work:

VH1-Anti A V_{H}

VH2-Anti B V_{H}

VK1-Anti C V_{K}

VK2-Anti D V_{K}

The procedures described above in Examples 1-3 may be used, in a similar manner as that described, to produce dimer molecules comprising combinations of V_{H} domains (i.e., V_{H}-V_{H} ligands) and combinations of V_{L} domains (V_{L}-V_{L} ligands).

### Example 4

### Creation and Characterisation of the Dual Specific ScFv Antibodies (VH1/VH2 Directed Against Antigens A and B and VK1/VK2 Directed Against Antigens C and D)

This example demonstrates that dual specific ScFv antibodies (VH1/VH2 directed against antigens A and B and VK1/VK2 directed against antigens C and D) could be created by combining V_{κ} and V_{H} single domains selected against respective antigens in a ScFv vector.

To create dual specific antibody VH1/VH2, VH1 single domain is excised from variable domain vector 1 (FIG. 7) by NcoI/XhoI digestion and ligated into NcoI/XhoI digested variable domain vector 2 (FIG. 7) to create VH1/variable domain vector 2. VH2 single domain is PCR amplified from variable domain vector 1 using primers to introduce SalI restriction site to the 5′ end and NotI restriction site to the 3′ end. The PCR product is then digested with SalI/NotI and ligated into SalI/NotI digested VH1/variable domain vector 2 to create VH1/VH2/variable domain vector 2.

VK1/VK2/variable domain vector 2 is created in a similar way. The dual specific nature of the produced VH1/VH2 ScFv and VK1/VK2 ScFv is tested in a soluble ScFv ELISA as described previously (see Example 6, PCT/GB 02/003014). Competition ELISA is performed as described previously (see Example 8, PCT/GB 02/003014).

Possible outcomes:

VH1/VH2 ScFv is able to bind antigens A and B simultaneously

VK1/VK2 ScFv is able to bind antigens C and D simultaneously

VH1/VH2 ScFv binding is competitive (when bound to antigen A, VH1/VH2 ScFv cannot bind to antigen B)

VK1/VK2 ScFv binding is competitive (when bound to antigen C, VK1/VK2 ScFv cannot bind to antigen D)

### Example 5

### Construction of Dual Specific VH1/VH2 Fab and VK1/VK2 Fab and Analysis of Their Binding Properties

To create VH1/VH2 Fab, VH1 single domain is ligated into NcoI/XhoI digested CH vector (FIG. 8) to create VH1/CH and VH2 single domain is ligated into SalI/NotI digested CK vector (FIG. 9) to create VH2/CK. Plasmid DNA from VH1/CH and VH2/CK is used to co-transform competent E. coli cells as described previously (see Example 8, PCT/GB02/003014).

The clone containing VH1/CH and VH2/CK plasmids is then induced by IPTG to produce soluble VH1/VH2 Fab as described previously (see Example 8, PCT/GB 02/003014).

VK1/VK2 Fab is produced in a similar way.

Binding properties of the produced Fabs are tested by competition ELISA as described previously (see Example 8, PCT/GB 02/003014).

Possible outcomes:

VH1/VH2 Fab is able to bind antigens A and B simultaneously

VK1/VK2 Fab is able to bind antigens C and D simultaneously

VH1/VH2 Fab binding is competitive (when bound to antigen A, VH1/VH2 Fab cannot bind to antigen B)

VK1/VK2 Fab binding is competitive (when bound to antigen C, VK1/VK2 Fab cannot bind to antigen D)

### Example 6

Chelating dAb Dimers

Summary

VH and VK homo-dimers are created in a dAb-linker-dAb format using flexible polypeptide linkers. Vectors were created in the dAb linker-dAb format containing glycine-serine linkers of different lengths 3U:(Gly₄Ser)₃, 5U:(Gly₄Ser)₅, 7U:(Gly₄Ser)₇.

Dimer libraries were created using guiding dAbs upstream of the linker: TAR1-5 (VK), TAR1-27(VK), TAR2-5(VH) or TAR2-6(VK) and a library of corresponding second dAbs after the linker. Using this method, novel dimeric dAbs were selected. The effect of dimerisation on antigen binding was determined by ELISA and BIAcore studies and in cell neutralisation and receptor binding assays. Dimerisation of both TAR1-5 and TAR1-27 resulted in significant improvement in binding affinity and neutralisation levels.

1.0 Methods

1.1 Library Generation

1.1.1 Vectors

pEDA3U, pEDA5U and pEDA7U vectors were designed to introduce different linker lengths compatible with the dAb-linker-dAb format. For pEDA3U, sense and anti-sense 73-base pair oligo linkers were annealed using a slow annealing program (95° C.-5 mins, 80° C.-10 mins, 70° C.-15 mins, 56° C.-15 mins, 42° C. until use) in buffer containing 0.1MNaCl, 10 mM Tris-HCl pH7.4 and cloned using the XhoI and NotI restriction sites. The linkers encompassed 3 (Gly₄Ser) units and a stuffer region housed between SalI and NotI cloning sites (scheme 1). In order to reduce the possibility of monomeric dAbs being selected for by phage display, the stuffer region was designed to include 3 stop codons, a SacI restriction site and a frame shift mutation to put the region out of frame when no second dAb was present. For pEDA5U and 7U due to the length of the linkers required, overlapping oligo-linkers were designed for each vector, annealed and elongated using Klenow. The fragment was then purified and digested using the appropriate enzymes before cloning using the XhoI and NotI restriction sites.

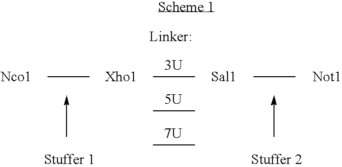

1.1.2 Library Preparation

The N-terminal V gene corresponding to the guiding dAb was cloned upstream of the linker using NcoI and XhoI restriction sites. VH genes have existing compatible sites, however cloning VK genes required the introduction of suitable restriction sites. This was achieved by using modifying PCR primers (VK-DLIBF: 5′ cggccatggcgtcaacggacat; VKXho1R: 5′ atgtgcgctcgagcgtttgattt 3′) in 30 cycles of PCR amplification using a 2:1 mixture of SuperTaq (HTBiotechnology Ltd) and pfu turbo (Stratagene). This maintained the NcoI site at the 5′ end while destroying the adjacent SalI site and introduced the XhoI site at the 3′ end. 5 guiding dAbs were cloned into each of the 3 dimer vectors: TAR1-5 (VK), TAR1-27(VK), TAR2-5(VH), TAR2-6(VK) and TAR2-7(VK). All constructs were verified by sequence analysis.

Having cloned the guiding dAbs upstream of the linker in each of the vectors (pEDA3U, 5U and 7U): TAR1-5 (VK), TAR1-27(VK), TAR2-5(VH) or TAR2-6(VK) a library of corresponding second dAbs were cloned after the linker. To achieve this, the complimentary dAb libraries were PCR amplified from phage recovered from round 1 selections of either a VK library against Human TNFα (at approximately 1×10⁶ diversity after round 1) when TAR1-5 or TAR1-27 are the guiding dAbs, or a VH or VK library against human p55 TNF receptor (both at approximately 1×10⁵ diversity after round 1) when TAR2-5 or TAR2-6 respectively are the guiding dAbs. For VK libraries PCR amplification was conducted using primers in 30 cycles of PCR amplification using a 2:1 mixture of SuperTaq and pfu turbo. VH libraries were PCR amplified using primers in order to introduce a SalI restriction site at the 5′ end of the gene. The dAb library PCRs were digested with the appropriate restriction enzymes, ligated into the corresponding vectors down stream of the linker, using SalI/NotI restriction sites and electroporated into freshly prepared competent TG1 cells.

The titres achieved for each library are as follows:

TAR1-5: pEDA3U=4×10⁸, pEDA5U=8×10⁷, pEDA7U=1×10⁸

TAR1-27: pEDA3U=6.2×10⁸, pEDA5U=1×10⁸, pEDA7U=1×10⁹

TAR2h-5: pEDA3U=4×10⁷, pEDA5U=2×10⁸, pEDA7U=8×10⁷

TAR2h-6: pEDA3U=7.4×10⁸, pEDA5U=1.2×10⁸, pEDA7U=2.2×10⁸

1.2 Selections

1.2.1 TNFα

Selections were conducted using human TNFα passively coated on immunotubes. Briefly, Immunotubes are coated overnight with 1-4 mls of the required antigen. The immunotubes were then washed 3 times with PBS and blocked with 2% milk powder in PBS for 1-2 hrs and washed a further 3 times with PBS. The phage solution is diluted in 2% milk powder in PBS and incubated at room temperature for 2 hrs. The tubes are then washed with PBS and the phage eluted with 1 mg/ml trypsin-PBS. Three selection strategies were investigated for the TAR1-5 dimer libraries. The first round selections were carried out in immunotubes using human TNFα coated at 1 μg/ml or 20 μg/ml with 20 washes in PBS 0.1% Tween. TG1 cells are infected with the eluted phage and the titres are determined (eg, Marks et al J Mol Biol. 1991 Dec. 5; 222(3):581-97, Richmann et al Biochemistry. 1993 Aug. 31; 32(34):8848-55).

The titres recovered were:

pEDA3U=2.8×10⁷ (1 μg/ml TNF) 1.5×10⁸ (20 μg/ml TNF),

pEDA5U=1.8×10⁷ (1 μg/ml TNF), 1.6×10⁸ (20 μg/ml TNF)

pEDA7U=8×10⁶ (1 μg/ml TNF), 7×10⁷ (20 μg/ml TNF).

The second round selections were carried out using 3 different methods.
1. In immunotubes, 20 washes with overnight incubation followed by a further 10 washes.
2. In immunotubes, 20 washes followed by 1 hr incubation at RT in wash buffer with (1 μg/ml TNFα) and 10 further washes.
3. Selection on streptavidin beads using 33 pmoles biotinylated human TNFα (Henderikx et al., 2002, Selection of antibodies against biotinylated antigens. Antibody Phage Display: Methods and protocols, Ed. O'Brien and Atkin, Humana Press). Single clones from round 2 selections were picked into 96 well plates and crude supernatant preps were made in 2 ml 96 well plate format.

| | Round 1 | | | |
|---|---|---|---|---|
| | Human | | | |
| | TNFαimmuno | Round 2 | Round 2 | Round 2 |
| | tube coating | selection | selection | selection |
| | concentration | method 1 | method 2 | method 3 |
| pEDA3U | 1 μg/ml | 1 × 10⁹ | 1.8 × 10⁹ | 2.4 × 10¹⁰ |
| pEDA3U | 20 μg/ml | 6 × 10⁹ | 1.8 × 10¹⁰ | 8.5 × 10¹⁰ |
| pEDA5U | 1 μg/ml | 9 × 10⁸ | 1.4 × 10⁹ | 2.8 × 10¹⁰ |
| pEDA5U | 20 μg/ml | 9.5 × 10⁹ | 8.5 × 10⁹ | 2.8 × 10¹⁰ |
| pEDA7U | 1 μg/ml | 7.8 × 10⁸ | 1.6 × 10⁸ | 4 × 10¹⁰ |
| pEDA7U | 20 μg/ml | 1 × 10¹⁰ | 8 × 10⁹ | 1.5 × 10¹⁰ |

For TAR1-27, selections were carried out as described previously with the following modifications. The first round selections were carried out in immunotubes using human TNFα coated at 1 μg/ml or 20 μg/ml with 20 washes in PBS 0.1% Tween. The second round selections were carried out in immunotubes using 20 washes with overnight incubation followed by a further 20 washes. Single clones from round 2 selections were picked into 96 well plates and crude supernatant preps were made in 2m196 well plate format.

TAR1-27 titres are as follows:

| | Human | | |
|---|---|---|---|
| | TNFαimmunotube | | |
| | coating conc | Round 1 | Round 2 |
| pEDA3U | 1 μg/ml | 4 × 10⁹ | 6 × 10⁹ |
| pEDA3U | 20 μg/ml | 5 × 10⁹ | 4.4 × 10¹⁰ |
| pEDA5U | 1 μg/ml | 1.5 × 10⁹ | 1.9 × 10¹⁰ |
| pEDA5U | 20 μg/ml | 3.4 × 10⁹ | 3.5 × 10¹⁰ |
| pEDA7U | 1 μg/ml | 2.6 × 10⁹ | 5 × 10⁹ |
| pEDA7U | 20 μg/ml | 7 × 10⁹ | 1.4 × 10¹⁰ |

1.2.2 TNF Receptor 1 (p55 Receptor; TAR2)

Selections were conducted as described previously for the TAR2h-5 libraries only. 3 rounds of selections were carried out in immunotubes using either 1 μg/ml human p55 TNF receptor or 10 μg/ml human p55 TNF receptor with 20 washes in PBS 0.1% Tween with overnight incubation followed by a further 20 washes. Single clones from round 2 and 3 selections were picked into 96 well plates and crude supernatant preps were made in 2 ml 96 well plate format.

TAR2h-5 titres are as follows:

| | Round 1 human | | | |
|---|---|---|---|---|
| | p55 TNF | | | |
| | receptor | | | |
| | immunotube | | | |
| | coating | | | |
| | concentration | Round 1 | Round 2 | Round 3 |
| pEDA3U | 1 μg/ml | 2.4 × 10⁶ | 1.2 × 10⁷ | 1.9 × 10⁹ |
| pEDA3U | 10 μg/ml | 3.1 × 10⁷ | 7 × 10⁷ | 1 × 10⁹ |
| pEDA5U | 1 μg/ml | 2.5 × 10⁶ | 1.1 × 10⁷ | 5.7 × 10⁸ |
| pEDA5U | 10 μg/ml | 3.7 × 10⁷ | 2.3 × 10⁸ | 2.9 × 10⁹ |
| pEDA7U | 1 μg/ml | 1.3 × 10⁶ | 1.3 × 10⁷ | 1.4 × 10⁹ |
| pEDA7U | 10 μg/ml | 1.6 × 10⁷ | 1.9 × 10⁷ | 3 × 10¹⁰ |

1.3 Screening

Single clones from round 2 or 3 selections were picked from each of the 3U, 5U and 7U libraries from the different selections methods, where appropriate. Clones were grown in 2xTY with 100 μg/ml ampicillin and 1% glucose overnight at 37° C. A 1/100 dilution of this culture was inoculated into 2 mls of 2xTY with 100 μg/ml ampicillin and 0.1% glucose in 2 ml, 96 well plate format and grown at 37° C. shaking until OD600 was approximately 0.9. The culture was then induced with 1 mM IPTG overnight at 30° C. The supernatants were clarified by centrifugation at 4000 rpm for 15 mins in a sorval plate centrifuge. The supernatant preps the used for initial screening.

1.3.1 ELISA

Binding activity of dimeric recombinant proteins was compared to monomer by Protein A/L ELISA or by antigen ELISA. Briefly, a 96 well plate is coated with antigen or Protein A/L overnight at 4° C. The plate washed with 0.05% Tween-PBS, blocked for 2 hrs with 2% Tween-PBS. The sample is added to the plate incubated for 1 hr at room temperature. The plate is washed and incubated with the secondary reagent for 1 hr at room temperature. The plate is washed and developed with TMB substrate. Protein A/L-HRP or India-HRP was used as a secondary reagent. For antigen ELISAs, the antigen concentrations used were 1 μg/ml in PBS for Human TNFα and human THF receptor 1. Due to the presence of the guiding dAb in most cases dimers gave a positive ELISA signal therefore off rate determination was examined by BIAcore.

1.3.2 BIAcore

BIAcore analysis was conducted for TAR1-5 and TAR2h-5 clones. For screening, Human TNFα was coupled to a CM5 chip at high density (approximately 10000 RUs). 50 μl of Human TNFα (50 μg/ml) was coupled to the chip at 50 min in acetate buffer—pH5.5. Regeneration of the chip following analysis using the standard methods is not possible due to the instability of Human TNFα, therefore after each sample was analysed, the chip was washed for 10 mins with buffer.

For TAR1-5, clones supernatants from the round 2 selection were screened by BIAcore.

48 clones were screened from each of the 3U, 5U and 7U libraries obtained using the following selection methods:

R1: 1 μg/ml human TNFα immunotube, R2 1 μg/ml human TNFα immunotube, overnight wash. R1: 20 μg/ml human TNFα immunotube, R2 20 μg/ml human TNFα immunotube, overnight wash.

R1: 1 μg/ml human TNFα immunotube, R2 33 pmoles biotinylated human TNFα on beads.

R1: 20 μg/ml human TNFα immunotube, R2 33 pmoles biotinylated human TNFα beads.

For screening, human p55 TNF receptor was coupled to a CM5 chip at high density (approximately 4000 RUs). 100 μl of human p55 TNF receptor (10 μg/ml ) was coupled to the chip at 5 μl/min in acetate buffer—pH5.5. Standard regeneration conditions were examined (glycine pH2 or pH3) but in each case antigen was removed from the surface of the chip therefore as with TNFα, therefore after each sample was analysed, the chip was washed for 10 mins with buffer.

For TAR2-5, clones supernatants from the round 2 selection were screened.

48 clones were screened from each of the 3U, 5U and 7U libraries, using the following selection methods:

R1: 1 μg/ml human p55 TNF receptor immunotube, R2 1 μg/ml human p55 TNF receptor immunotube, overnight wash.

R1: 10 μg/ml human p55 TNF receptor immunotube, R2 10 μg/ml human p55 TNF receptor immunotube, overnight wash.

1.3.3 Receptor and Cell Assays

The ability of the dimers to neutralise in the receptor assay was conducted as follows:

Receptor Binding

Anti-TNF dAbs were tested for the ability to inhibit the binding of TNF to recombinant TNF receptor 1 (p55). Briefly, Maxisorp plates were incubated overnight with 30 mg/ml anti-human Fc mouse monoclonal antibody (Zymed, San Francisco, USA). The wells were washed with phosphate buffered saline (PBS) containing 0.05% Tween-20 and then blocked with 1% BSA in PBS before being incubated with 100 ng/ml TNF receptor 1 Fc fusion protein (R&D Systems, Minneapolis, USA). Anti-TNF dAb was mixed with TNF which was added to the washed wells at a final concentration of 10 ng/ml. TNF binding was detected with 0.2 mg/ml biotinylated anti-TNF antibody (HyCult biotechnology, Uben, Netherlands) followed by 1 in 500 dilution of horse radish peroxidase labelled streptavidin (Amersham Biosciences, UK) and then incubation with TMB substrate (KPL, Gaithersburg, USA). The reaction was stopped by the addition of HCl and the absorbance was read at 450 nm. Anti-TNF dAb activity lead to a decrease in TNF binding and therefore a decrease in absorbance compared with the TNF only control.

L929 Cytotoxicity Assay

Anti-TNF dAbs were also tested for the ability to neutralise the cytotoxic activity of TNF on mouse L929 fibroblasts (Evans, T. (2000) Molecular Biotechnology 15, 243-248). Briefly, L929 cells plated in microtitre plates were incubated overnight with anti-TNF dAb, 100 pg/ml TNF and 1 mg/ml actinomycin D (Sigma, Poole, UK). Cell viability was measured by reading absorbance at 490 nm following an incubation with [3-(4,5-dimethylthiazol-2-yl)-5-(3-carbboxymethoxyphenyl)-2-(4-sulfophenyl)-2H-tetrazolium (Promega, Madison, USA). Anti-TNF dAb activity lead to a decrease in TNF cytotoxicity and therefore an increase in absorbance compared with the TNF only control.

In the initial screen, supernatants prepared for BIAcore analysis, described above, were also used in the receptor assay. Further analysis of selected dimers was also conducted in the receptor and cell assays using purified proteins.

HeLa IL-8 Assay

Anti-TNFRI or anti-TNF alpha dAbs were tested for the ability to neutralise the induction of IL-8 secretion by TNF in HeLa cells (method adapted from that of Akeson, L. et al (1996) Journal of Biological Chemistry 271, 30517-30523, describing the induction of IL-8 by IL-1 in HUVEC; here we look at induction by human TNF alpha and we use HeLa cells instead of the HUVEC cell line). Briefly, HeLa cells plated in microtitre plates were incubated overnight with dAb and 300 pg/ml TNF. Post incubation the supernatant was aspirated off the cells and IL-8 concentration measured via a sandwich ELISA (R&D Systems). Anti-TNFRI dAb activity lead to a decrease in IL-8 secretion into the supernatant compared with the TNF only control.

The L929 assay is used throughout the following experiments; however, the use of the HeLa IL-8 assay is preferred to measure anti-TNF receptor 1 (p55) ligands; the presence of mouse p55 in the L929 assay poses certain limitations in its use.

1.4 Sequence Analysis

Dimers that proved to have interesting properties in the BIAcore and the receptor assay screens were sequenced. Sequences are detailed in the sequence listing.

1.5 Formatting

1.5.1 TAR1-5-19 Dimers

The TAR1-5 dimers that were shown to have good neutralisation properties were re-formatted and analysed in the cell and receptor assays. The TAR1-5 guiding dab was substituted with the affinity matured clone TAR1-5-19. To achieve this TAR1-5 was cloned out of the individual dimer pair and substituted with TAR1-5-19 that had been amplified by PCR. In addition, TAR1-5-19 homodimers were also constructed in the 3U, 5U and 7U vectors. The N terminal copy of the gene was amplified by PCR and cloned as described above and the C-terminal gene fragment was cloned using existing SalI and NotI restriction sites.

1.5.2 Mutagenesis

The amber stop codon present in dAb2, one of the C-terminal dAbs in the TAR1-5 dimer pairs was mutated to a glutamine by site-directed mutagenesis.

1.5.3 Fabs

The dimers containing TAR1-5 or TAR1-5-19 were re-formatted into Fab expression vectors. dAbs were cloned into expression vectors containing either the CK or CH genes using SfiI and NotI restriction sites and verified by sequence analysis. The CK vector is derived from a pUC based ampicillin resistant vector and the CH vector is derived from a pACYC chloramphenicol resistant vector. For Fab expression the dAb-CH and dAb-CK constructs were co-transformed into HB2151 cells and grown in 2xTY containing 0.1% glucose, 100 μg/ml ampicillin and 10 μg/ml chloramphenicol.

1.5.3 Hinge Dimerisation

Dimerisation of dAbs via cystine bond formation was examined. A short sequence of amino acids EPKSGDKTHTCPPCP a modified form of the human IgGC1 hinge was engineered at the C terminal region on the dAb. An oligo linker encoding for this sequence was synthesised and annealed, as described previously. The linker was cloned into the pEDA vector containing TAR1-5-19 using XhoI and NotI restriction sites. Dimerisation occurs in situ in the periplasm.

1.6 Expression and Purification

1.6.1 Expression

Supernatants were prepared in the 2 ml, 96-well plate format for the initial screening as described previously. Following the initial screening process selected dimers were analysed further. Dimer constructs were expressed in TOP10F′ or HB2151 cells as supernatants. Briefly, an individual colony from a freshly streaked plate was grown overnight at 37° C. in 2xTY with 100 μg/ml ampicillin and 1% glucose. A 1/100 dilution of this culture was inoculated into 2xTY with 100 μg/ml ampicillin and 0.1% glucose and grown at 37° C. shaking until OD600 was approximately 0.9. The culture was then induced with 1 mM IPTG overnight at 30° C. The cells were removed by centrifugation and the supernatant purified with protein A or L agarose.

Fab and cysteine hinge dimers were expressed as periplasmic proteins in HB2152 cells. A 1/100 dilution of an overnight culture was inoculated into 2xTY with 0.1% glucose and the appropriate antibiotics and grown at 30° C. shaking until OD600 was approximately 0.9. The culture was then induced with 1 mM IPTG for 3-4 hours at 25° C. The cells were harvested by centrifugation and the pellet resuspended in periplasmic preparation buffer (30 mM Tris-HCl pH8.0, 1 mM EDTA, 20% sucrose). Following centrifugation the supernatant was retained and the pellet resuspended in 5 mM MgSO₄. The supernatant was harvested again by centrifugation, pooled and purified.

1.6.2 Protein A/L Purification

Optimisation of the purification of dimer proteins from Protein L agarose (Affitech, Norway) or Protein A agarose (Sigma, UK) was examined. Protein was eluted by batch or by column elution using a peristaltic pump. Three buffers were examined 0.1M Phosphate-citrate buffer pH2.6, 0.2M Glycine pH2.5 and 0.1M Glycine pH2.5. The optimal condition was determined to be under peristaltic pump conditions using 0.1M Glycine pH2.5 over 10 column volumes. Purification from protein A was conducted peristaltic pump conditions using 0.1M Glycine pH2.5.

1.6.3 FPLC Purification

Further purification was carried out by FPLC analysis on the AKTA Explorer 100 system (Amersham Biosciences Ltd). TAR1-5 and TAR1-5-19 dimers were fractionated by cation exchange chromatography (1 ml Resource S—Amersham Biosciences Ltd) eluted with a 0-1M NaCl gradient in 50 mM acetate buffer pH4. Hinge dimers were purified by ion exchange (1 ml Resource Q Amersham Biosciences Ltd) eluted with a 0-1M NaCl gradient in 25 mMTris HCl pH 8.0. Fabs were purified by size exclusion chromatography using a superose 12 (Amersham Biosciences Ltd) column run at a flow rate of 0.5 ml /min in PBS with 0.05% tween. Following purification samples were concentrated using vivaspin 5K cut off concentrators (Vivascience Ltd).

2.0 Results

2.1 TAR1-5 Dimers

6×96 clones were picked from the round 2 selection encompassing all the libraries and selection conditions. Supernatant preps were made and assayed by antigen and Protein L ELISA, BIAcore and in the receptor assays. In ELISAs, positive binding clones were identified from each selection method and were distributed between 3U, 5U and 7U libraries. However, as the guiding dAb is always present it was not possible to discriminate between high and low affinity binders by this method therefore BIAcore analysis was conducted.

BIAcore analysis was conducted using the 2 ml supernatants. BIAcore analysis revealed that the dimer Koff rates were vastly improved compared to monomeric TAR1-5. Monomer Koff rate was in the range of 10⁻¹M compared with dimer Koff rates which were in the range of 10⁻³-10⁻⁴M. 16 clones that appeared to have very slow off rates were selected, these came from the 3U, 5U and 7U libraries and were sequenced. In addition the supernatants were analysed for the ability to neutralise human TNFα in the receptor assay.

6 lead clones (d1-d6 below) that neutralised in these assays and have been sequenced. The results shows that out of the 6 clones obtained there are only 3 different second dAbs (dAb1, dAb2 and dAb3) however where the second dAb is found more than once they are linked with different length linkers.

TAR1-5d1: 3U linker 2^{nd} dAb=dAb1-1 μg/ml Ag immunotube overnight wash

TAR1-5d2: 3U linker 2^{nd} dAb=dAb2-1 μg/ml Ag immunotube overnight wash

TAR1-5d3: 5U linker 2^{nd} dAb=dAb2-1 μg/ml Ag immunotube overnight wash

TAR1-5d4: 5U linker 2^{nd} dAb=dAb3-20 μg/ml Ag immunotube overnight wash

TAR1-5d5: 5U linker 2^{nd} dAb=dAb1-20 μg/ml Ag immunotube overnight wash

TAR1-5d6: 7U linker 2^{nd} dAb=dAb1-R1:1 μg/ml Ag immunotube overnight wash,

R2:beads

The 6 lead clones were examined further. Protein was produced from the periplasm and supernatant, purified with protein L agarose and examined in the cell and receptor assays. The levels of neutralisation were variable (Table 1). The optimal conditions for protein preparation were determined. Protein produced from HB2151 cells as supernatants gave the highest yield (approximately 10 mgs/L of culture). The supernatants were incubated with protein L agarose for 2 hrs at room temperature or overnight at 4° C. The beads were washed with PBS/NaCl and packed onto an FPLC column using a peristaltic pump. The beads were washed with 10 column volumes of PBS/NaCl and eluted with 0.1M glycine pH2.5. In general, dimeric protein is eluted after the monomer.

TAR1-5d1-6 dimers were purified by FPLC. Three species were obtained, by FPLC purification and were identified by SDS PAGE. One species corresponds to monomer and the other two species corresponds to dimers of different sizes. The larger of the two species is possibly due to the presence of C terminal tags. These proteins were examined in the receptor assay. The data presented in table 1 represents the optimum results obtained from the two dimeric species (FIG. 11)

The three second dAbs from the dimer pairs (ie, dAb1, dAb2 and dAb3) were cloned as monomers and examined by ELISA and in the cell and receptor assay. All three dAbs bind specifically to TNF by antigen ELISA and do not cross react with plastic or BSA. As monomers, none of the dAbs neutralise in the cell or receptor assays.

2.1.2 TAR1-5-19 Dimers

TAR1-5-19 was substituted for TAR1-5 in the 6 lead clones. Analysis of all TAR1-5-19 dimers in the cell and receptor assays was conducted using total protein (protein L purified only) unless otherwise stated (Table 2). TAR1-5-19d4 and TAR1-5-19d3 have the best ND₅₀ (˜5 nM) in the cell assay, this is consistent with the receptor assay results and is an improvement over TAR1-5-19 monomer (ND₅₀˜30 nM). Although purified TAR1-5 dimers give variable results in the receptor and cell assays TAR1-5-19 dimers were more consistent. Variability was shown when using different elution buffers during the protein purification. Elution using 0.1M Phosphate-citrate buffer pH2.6 or 0.2M Glycine pH2.5 although removing all protein from the protein L agarose in most cases rendered it less functional.

TAR1-5-19d4 was expressed in the fermenter and purified on cation exchange FPLC to yield a completely pure dimer. As with TAR1-5d4 three species were obtained, by FPLC purification corresponding to monomer and two dimer species. This dimer was amino acid sequenced. TAR1-5-19 monomer and TAR1-5-19d4 were then examined in the receptor assay and the resulting IC50 for monomer was 30 nM and for dimer was 8 nM. The results of the receptor assay comparing TAR1-5-19 monomer, TAR1-5-19d4 and TAR1-5d4 is shown in FIG. 10.

TAR1-5-19 homodimers were made in the 3U, 5U and 7U vectors, expressed and purified on Protein L. The proteins were examined in the cell and receptor assays and the resulting IC₅₀s (for receptor assay) and ND₅₀s (for cell assay) were determined (table 3, FIG. 12).

2.2 Fabs

TAR1-5 and TAR1-5-19 dimers were also cloned into Fab format, expressed and purified on protein L agarose. Fabs were assessed in the receptor assays (Table 4). The results showed that for both TAR1-5-19 and TAR1-5 dimers the neutralisation levels were similar to the original Gly₄Ser linker dimers from which they were derived. A TAR1-5-19 Fab where TAR1-5-19 was displayed on both CH and CK was expressed, protein L purified and assessed in the receptor assay. The resulting IC50 was approximately 1 nM.

2.3 TAR1-27 Dimers

3×96 clones were picked from the round 2 selection encompassing all the libraries and selection conditions. 2 ml supernatant preps were made for analysis in ELISA and bioassays. Antigen ELISA gave 71 positive clones. The receptor assay of crude supernatants yielded 42 clones with inhibitory properties (TNF binding 0-60%). In the majority of cases inhibitory properties correlated with a strong ELISA signal. 42 clones were sequenced, 39 of these have unique second dAb sequences. The 12 dimers that gave the best inhibitory properties were analysed further.

The 12 neutralising clones were expressed as 200 ml supernatant preps and purified on protein L. These were assessed by protein L and antigen ELISA, BIAcore and in the receptor assay. Strong positive ELISA signals were obtained in all cases. BIAcore analysis revealed all clones to have fast on and off rates. The off rates were improved compared to monomeric TAR1-27, however the off rate of TAR1-27 dimers was faster (Koff is approximately in the range of 10⁻¹ and 10⁻²M) than the TAR1-5 dimers examined previously (Koff is approximately in the range of 10⁻³-10⁻⁴M). The stability of the purified dimers was questioned and therefore in order to improve stability, the addition on 5% glycerol, 0.5% Triton X100 or 0.5% NP40 (Sigma) was included in the purification of 2 TAR1-27 dimers (d2 and d16). Addition of NP40 or Triton X100™ improved the yield of purified product approximately 2 fold. Both dimers were assessed in the receptor assay. TAR1-27d2 gave IC50 of ˜30 nM under all purification conditions. TAR1-27d16 showed no neutralisation effect when purified without the use of stabilising agents but gave an IC50 of ˜50 nM when purified under stabilising conditions. No further analysis was conducted.

2.4 TAR2-5 Dimers

3×96 clones were picked from the second round selections encompassing all the libraries and selection conditions. 2 ml supernatant preps were made for analysis. Protein A and antigen ELISAs were conducted for each plate. 30 interesting clones were identified as having good off-rates by BIAcore (Koff ranges between 10⁻²-10⁻³M). The clones were sequenced and 13 unique dimers were identified by sequence analysis.

TABLE 1
TAR1-5 dimers

| | | | | | Receptor/ |
|---|---|---|---|---|---|
| | | | | Elution | Cell |
| Dimer | Cell type | Purification | Protein Fraction | conditions | assay |
| TAR1-5d1 | HB2151 | Protein L + | small dimeric | 0.1M glycine | RA~30 nM |
| | | FPLC | species | pH 2.5 | |
| TAR1-5d2 | HB2151 | Protein L + | small dimeric | 0.1M glycine | RA~50 nM |
| | | FPLC | species | pH 2.5 | |
| TAR1-5d3 | HB2151 | Protein L + | large dimeric | 0.1M glycine | RA~300 nM |
| | | FPLC | species | pH 2.5 | |
| TAR1-5d4 | HB2151 | Protein L + | small dimeric | 0.1M glycine | RA~3 nM |
| | | FPLC | species | pH 2.5 | |
| TAR1-5d5 | HB2151 | Protein L + | large dimeric | 0.1M glycine | RA~200 nM |
| | | FPLC | species | pH 2.5 | |
| TAR1-5d6 | HB2151 | Protein L + | Large dimeric | 0.1M glycine | RA~100 nM |
| | | FPLC | species | pH 2.5 | |

*note
dimer 2 and dimer 3 have the same second dAb (called dAb2), however have different linker lengths (d2 = (Gly₄Ser)₃, d3 = (Gly₄Ser)₃). dAb1 is the partner dAb to dimers 1, 5 and 6. dAb3 is the partner dAb to dimer 4. None of the partner dAbs neutralise alone.
FPLC purification is by cation exchange unless otherwise stated.
The optimal dimeric species for each dimer obtained by FPLC was determined in these assays.

TABLE 2
TAR1-5-19 dimers

| | | | | | Receptor/ |
|---|---|---|---|---|---|
| | | | Protein | | Cell |
| Dimer | Cell type | Purification | Fraction | Elution conditions | assay |
| TAR1-5-19 d1 | TOP10F′ | Protein L | Total protein | 0.1M glycine pH 2.0 | RA~15 nM |
| TAR1-5-19 d2 (no | TOP10F′ | Protein L | Total protein | 0.1M glycine pH 2.0 + | RA~2 nM |
| stop codon) | | | | 0.05% NP40 | |
| TAR1-5-19d3 | TOP10F′ | Protein L | Total protein | 0.1M glycine pH 2.5 + | RA~8 nM |
| (no stop codon) | | | | 0.05% NP40 | |
| TAR1-5-19d4 | TOP10F′ | Protein L + | FPLC purified | 0.1M glycine | RA~2-5 nM |
| | | FPLC | fraction | pH 2.0 | CA~12 nM |
| TAR1-5-19d5 | TOP10F′ | Protein L | Total protein | 0.1M glycine pH 2.0 + | RA~8 nM |
| | | | | NP40 | CA~10 nM |
| TAR1-5-19 d6 | TOP10F′ | Protein L | Total protein | 0.1M glycine pH 2.0 | RA~10 nM |

TABLE 3
TAR1-5-19 homodimers

| | | | | | Receptor/ |
|---|---|---|---|---|---|
| | | | | | Cell |
| Dimer | Cell type | Purification | Protein Fraction | Elution conditions | assay |
| TAR1-5-19 3U | HB2151 | Protein L | Total protein | 0.1M glycine pH 2.5 | RA~20 nM |
| homodimer | | | | | CA~30 nM |
| TAR1-5-19 5U | HB2151 | Protein L | Total protein | 0.1M glycine pH 2.5 | RA~2 nM |
| homodimer | | | | | CA~3 nM |
| TAR1-5-19 7U | HB2151 | Protein L | Total protein | 0.1M glycine pH 2.5 | RA~10 nM |
| homodimer | | | | | CA~15 nM |
| TAR1-5-19 cys | HB2151 | Protein L + FPLC | FPLC purified | 0.1M glycine pH 2.5 | RA~2 nM |
| hinge | | | dimer fraction | | |
| TAR1-5-19CH/ | HB2151 | Protein | Total protein | 0.1M glycine pH 2.5 | RA~1 nM |
| TAR1-5-19 CK | | | | | |

TABLE 4
TAR1-5/TAR1-5-19 Fabs

| | | | | | Receptor/ |
|---|---|---|---|---|---|
| | Cell | | Protein | Elution | Cell |
| Dimer | type | Purification | Fraction | conditions | assay |
| TAR1-5CH/ | HB2151 | Protein L | Total protein | 0.1M citrate pH 2.6 | RA~90 nM |
| dAb1 CK | | | | | |
| TAR1-5CH/ | HB2151 | Protein L | Total protein | 0.1M glycine pH 2.5 | RA~30 nM |
| dAb2 CK | | | | | CA-60 nM |
| dAb3CH/ | HB2151 | Protein L | Total protein | 0.1M citrate pH 2.6 | RA~100 nM |
| TAR1-5CK | | | | | |
| TAR1-5-19CH/ | HB2151 | Protein L | Total protein | 0.1M glycine pH 2.0 | RA~6 nM |
| dAb1 CK | | | | | |
| dAb1 CH/ | HB2151 | Protein L | 0.1M glycine | Myc/flag | RA~6 nM |
| TAR1-5-19CK | | | pH 2.0 | | |
| TAR1-5-19CH/ | HB2151 | Protein L | Total protein | 0.1M glycine pH 2.0 | RA~8 nM |
| dAb2 CK | | | | | CA~12 nM |
| TAR1-5-19CH/ | HB2151 | Protein L | Total protein | 0.1M glycine pH 2.0 | RA~3 nM |
| dAb3CK | | | | | |

### Example 7

dAb Dimerisation by Terminal Cysteine Linkage

Summary

For dAb dimerisation, a free cysteine has been engineered at the C-terminus of the protein. When expressed the protein forms a dimer which can be purified by a two step purification method.

PCR Construction of TAR1-5-19CYS Dimer

See example 8 describing the dAb trimer. The trimer protocol gives rise to a mixture of monomer, dimer and trimer.

Expression and Purification of TAR1-5-19CYS Dimer

The dimer was purified from the supernatant of the culture by capture on Protein L agarose as outlined in the example 8.

Separation of TAR1-5-19CYS Monomer from the TAR1-5-19CYS Dimer

Prior to cation exchange separation, the mixed monomer/dimer sample was buffer exchanged into 50 mM sodium acetate buffer pH 4.0 using a PD-10 column (Amersham Pharmacia), following the manufacturer's guidelines. The sample was then applied to a 1 mL Resource S cation exchange column (Amersham Pharmacia), which had been pre-equilibrated with 50 mM sodium acetate pH 4.0. The monomer and dimer were separated using the following salt gradient in 50 mM sodium acetate pH 4.0:

150 to 200 mM sodium chloride over 15 column volumes

200 to 450 mM sodium chloride over 10 column volumes

450 to 1000 mM sodium chloride over 15 column volumes

Fractions containing dimer only were identified using SDS-PAGE and then pooled and the pH increased to 8 by the addition of ⅕ volume of 1M Tris pH 8.0.

In Vitro Functional Binding Assay: TNF Receptor Assay and Cell Assay

The affinity of the dimer for human TNFα was determined using the TNF receptor and cell assay. IC50 in the receptor assay was approximately 0.3-0.8 nM; ND50 in the cell assay was approximately 3-8 nM.

Other Possible TAR1-5-19CYS Dimer Formats

PEG Dimers and Custom Synthetic Maleimide Dimers

Nektar (Shearwater) offer a range of bi-maleimide PEGs [mPEG2-(MAL)2 or mPEG-(MAL)2] which would allow the monomer to be formatted as a dimer, with a small linker separating the dAbs and both being linked to a PEG ranging in size from 5 to 40 kDa. It has been shown that the 5 kDa mPEG-(MAL)2 (ie, [TAR1-5-19]-Cys-maleimide-PEG×2, wherein the maleimides are linked together in the dimer) has an affinity in the TNF receptor assay of ˜1-3 nM. Also the dimer can also be produced using TMEA (Tris[2-maleimidoethyl]amine) (Pierce Biotechnology) or other bi-functional linkers.

It is also possible to produce the disulphide dimer using a chemical coupling procedure using 2,2′-dithiodipyridine (Sigma Aldrich) and the reduced monomer.

Addition of a Polypeptide Linker or Hinge to the C-Terminus of the dAb.

A small linker, either (Gly₄Ser)ₙ where n=1 to 10, eg, 1, 2, 3, 4, 5, 6 or 7, an immunoglobulin (eg, IgG hinge region or random peptide sequence (eg, selected from a library of random peptide sequences) can be engineered between the dAb and the terminal cysteine residue. This can then be used to make dimers as outlined above.

### Example 8

dAb Trimerisation

Summary

For dAb trimerisation, a free cysteine is required at the C-terminus of the protein. The cysteine residue, once reduced to give the free thiol, can then be used to specifically couple the protein to a trimeric maleimide molecule, for example TMEA (Tris[2-maleimidoethyl]amine).

PCR Construction of TAR1-5-19CYS

The following oligonucleotides were used to specifically PCR TAR1-5-19 with a SalI and BamHI sites for cloning and also to introduce a C-terminal cysteine residue:

| | |
|---|---|
| | SalI |
| | ~~~~~~~~ |
| | Trp Ser Ala Ser Thr Asp* Ile Gln Met Thr Gln Ser Pro Ser Ser Leu Ser Ala Ser Val |
| 1 | TGG AGC GCG TCG ACG GAC ATG CAG ATG ACC CAG TCT CCA TCC TCT CTG TCT GCA TCT GTA |
| | ACC TCG CGC AGC TGC CTG TAG GTC TAC TGG GTC AGA GGT AGG AGA GAC AGA CGT AGA CAT |
| | Gly Asp Arg Val Thr Ile Thr Cys Arg Ala Ser Gln Ser Ile Asp Ser Tyr Leu His Trp |
| 61 | GGA GAC CGT GTC ACC ATC ACT TGC CGG GCA AGT CAG AGC ATT GAT AGT TAT TTA CAT TGG |
| | CCT CTG GCA CAG TGG TAG TGA ACG GCC CGT TCA GTC TCG TAA CTA TCA ATA AAT GTA ACC |
| | Tyr Gln Gln Lys Pro Gly Lys Ala Pro Lys Leu Leu Ile Tyr Ser Ala Ser Glu Leu Gln |
| 121 | TAC CAG CAG AAA CCA GGG AAA GCC CCT AAG CTC CTG ATC TAT AGT GCA TCC GAG TTG CAA |
| | ATG GTC GTC TTT GGT CCC TTT CGG GGA TTC GAG GAC TAG ATA TCA CGT AGG CTC AAC GTT |
| | Ser Gly Val Pro Ser Arg Phe Ser Gly Ser Gly Ser Gly Thr Asp Phe Thr Leu Thr Ile |
| 181 | AGT GGG GTC CCA TCA CGT TTC AGT GGC AGT GGA TCT GGG ACA GAT TTC ACT CTC ACC ATC |
| | TCA CCC CAG GGT AGT GCA AAG TCA CCG TCA CCT AGA CCC TGT CTA AAG TGA GAG TGG TAG |
| | Ser Ser Leu Gln Pro Glu Asp Phe Ala Thr Tyr Tyr Cys Gln Gln Val Val Trp Arg Pro |
| 241 | AGC AGT CTG CAA CCT GAA GAT TTT GCT ACG TAC TAC TGT CAA CAG GTT GTG TGG CGT CCT |
| | TCG TCA GAC GTT GGA CTT CTA AAA CGA TGC ATG ATG ACA GTT GTC CAA CAC ACC GCA GCA |
| | BamHI |
| | ~~~~~~~~ |
| | Phe Thr Phe Gly Gln Gly Thr Lys Val Glu Ile Lys Arg Cys *** *** Gly Ser Gly |
| 301 | TTT ACG TTC GGC CAA GGG ACC AAG GTG GAA ATC AAA CGG TGC TAA TAA GGA TCC GGC |
| | AAA TGC AAG CCG GTT CCC TGG TTC CAC CTT TAG TTT GCC ACG ATT ATT CCT AGG CCG |

(* start of TAR1-5-19CYS sequence)
Forward primer
5′-TGGAGCGCGTCGACGGACATCCAGATGACCCAGTCTCCA-3′
Reverse primer
5′-TTAGCAGCCGGATCCTTATTAGCACCGTTTGATTTCCAC-3′

The PCR reaction (504 volume) was set up as follows: 200 μM dNTPs, 0.4 μM of each primer, 5 μL of 10×Pfu Turbo buffer (Stratagene), 100 ng of template plasmid (encoding TAR1-5-19), 14 of Pfu Turbo enzyme (Stratagene) and the volume adjusted to 50 μL using sterile water. The following PCR conditions were used: initial denaturing step 94° C. for 2 mins, then 25 cycles of 94° C. for 30 secs, 64° C. for 30 sec and 72° C. for 30 sec. A final extension step was also included of 72° C. for 5 mins. The PCR product was purified and digested with SalI and BamHI and ligated into the vector which had also been cut with the same restriction enzymes. Correct clones were verified by DNA sequencing.

Expression and Purification of TAR1-5-19CYS

TAR1-5-19CYS vector was transformed into BL21 (DE3) pLysS chemically competent cells (Novagen) following the manufacturer's protocol. Cells carrying the dAb plasmid were selected for using 100 μg/mL carbenicillin and 37 μg/mL chloramphenicol. Cultures were set up in 2L baffled flasks containing 500 mL of terrific broth (Sigma-Aldrich), 100 μg/mL carbenicillin and 37 μg/mL chloramphenicol. The cultures were grown at 30° C. at 200 rpm to an O.D.600 of 1-1.5 and then induced with 1 mM IPTG (isopropyl-beta-D-thiogalactopyranoside, from Melford Laboratories). The expression of the dAb was allowed to continue for 12-16 hrs at 30° C. It was found that most of the dAb was present in the culture media. Therefore, the cells were separated from the media by centrifugation (8,000×g for 30 mins), and the supernatant used to purify the dAb. Per litre of supernatant, 30 mL of Protein L agarose (Affitech) was added and the dAb allowed to batch bind with stirring for 2 hours. The resin was then allowed to settle under gravity for a further hour before the supernatant was siphoned off. The agarose was then packed into a XK 50 column (Amersham Phamacia) and was washed with 10 column volumes of PBS. The bound dAb was eluted with 100 mM glycine pH 2.0 and protein containing fractions were then neutralized by the addition of ⅕ volume of 1 M Tris pH 8.0. Per litre of culture supernatant 20 mg of pure protein was isolated, which contained a 50:50 ratio of monomer to dimer.

Trimerisation of TAR1-5-19CYS

2.5 ml of 100 μM TAR1-5-19CYS was reduce with 5 mM dithiothreitol and left at room temperature for 20 minutes. The sample was then buffer exchanged using a PD-10 column (Amersham Pharmacia). The column had been pre-equilibrated with 5 mM EDTA, 50 mM sodium phosphate pH 6.5, and the sample applied and eluted following the manufactures guidelines. The sample was placed on ice until required. TMEA (Tris[2-maleimidoethyl]amine) was purchased from Pierce Biotechnology. A 20 mM stock solution of TMEA was made in 100% DMSO (dimethyl sulphoxide). It was found that a concentration of TMEA greater than 3:1 (molar ratio of dAb:TMEA) caused the rapid precipitation and cross-linking of the protein. Also the rate of precipitation and cross-linking was greater as the pH increased. Therefore using 100 μM reduced TAR1-5-19CYS, 25 μM TMEA was added to trimerise the protein and the reaction allowed to proceed at room temperature for two hours. It was found that the addition of additives such as glycerol or ethylene glycol to 20% (v/v), significantly reduced the precipitation of the trimer as the coupling reaction proceeded. After coupling, SDS-PAGE analysis showed the presence of monomer, dimer and trimer in solution.

Purification of the Trimeric TAR1-5-19CYS

40 μL of 40% glacial acetic acid was added per mL of the TMEA-TAR1-5-19cys reaction to reduce the pH to ˜4. The sample was then applied to a 1 mL Resource S cation exchange column (Amersham Pharmacia), which had been pre-equilibrated with 50 mM sodium acetate pH 4.0. The dimer and trimer were partially separated using a salt gradient of 340 to 450 mM Sodium chloride, 50 mM sodium acetate pH 4.0 over 30 column volumes. Fractions containing trimer only were identified using SDS-PAGE and then pooled and the pH increased to 8 by the addition of ⅕ volume of 1M Tris pH 8.0. To prevent precipitation of the trimer during concentration steps (using 5K cut off Viva spin concentrators; Vivascience), 10% glycerol was added to the sample.

In Vitro Functional Binding Assay: TNF Receptor Assay and Cell Assay

The affinity of the trimer for human TNFα was determined using the TNF receptor and cell assay. IC50 in the receptor assay was 0.3 nM; ND50 in the cell assay was in the range of 3 to 10 nM (eg, 3 nM).

Other Possible TAR1-5-19CYS Trimer Formats

TAR1-5-19CYS may also be formatted into a trimer using the following reagents:

PEG Trimers and Custom Synthetic Maleimide Trimers

Nektar (Shearwater) offer a range of multi arm PEGs, which can be chemically modified at the terminal end of the PEG. Therefore using a PEG trimer with a maleimide functional group at the end of each arm would allow the trimerisation of the dAb in a manner similar to that outlined above using TMEA. The PEG may also have the advantage in increasing the solubility of the trimer thus preventing the problem of aggregation. Thus, one could produce a dAb trimer in which each dAb has a C-terminal cysteine that is linked to a maleimide functional group, the maleimide functional groups being linked to a PEG trimer.

Addition of a Polypeptide Linker or Hinge to the C-Terminus of the dAb

A small linker, either (Gly₄Ser)ₙ where n=1 to 10, eg, 1, 2, 3, 4, 5, 6 or 7 , an immunoglobulin (eg, IgG hinge region or random peptide sequence (eg, selected from a library of random peptide sequences) could be engineered between the dAb and the terminal cysteine residue. When used to make multimers (eg, dimers or trimers), this again would introduce a greater degree of flexibility and distance between the individual monomers, which may improve the binding characteristics to the target, eg a multisubunit target such as human TNFα.

### Example 9

Selection of a Collection of Single Domain Antibodies (dAbs) Directed Against Human Serum Albumin (HSA) and Mouse Serum Albumin (MSA).

This example explains a method for making a single domain antibody (dAb) directed against serum albumin. Selection of dAbs against both mouse serum albumin (MSA) and human serum albumin (HSA) is described. Three human phage display antibody libraries were used in this experiment, each based on a single human framework for V_{H} (see FIG. 13: sequence of dummy V_{H} based on V3-23/DP47 and JH4b) or V_{κ} (see FIG. 15: sequence of dummy V_{κ} based on o12/o2/DPK9 and Jk1) with side chain diversity encoded by NNK codons incorporated in complementarity determining regions (CDR1, CDR2 and CDR3).

Library 1 (V_{H}):

Diversity at positions: H30, H31, H33, H35, H50, H52, H52a, H53, H55, H56, H58, H95, H97, H98.

Library size: 6.2×10⁹

Library 2 (V_{H}):

Diversity at positions: H30, H31, H33, H35, H50, H52, H52a, H53, H55, H56, H58, H95, H97, H98, H99, H100, H100a, H100b.

Library size: 4.3×10⁹

Library 3 (V_{κ}):

Diversity at positions: L30, L31, L32, L34, L50, L53, L91, L92, L93, L94, L96

Library size: 2×10⁹

The V_{H} and V_{κ} libraries have been preselected for binding to generic ligands protein A and protein L respectively so that the majority of clones in the unselected libraries are functional. The sizes of the libraries shown above correspond to the sizes after preselection.

Two rounds of selection were performed on serum albumin using each of the libraries separately. For each selection, antigen was coated on immunotube (nunc) in 4 ml of PBS at a concentration of 100 μg/ml. In the first round of selection, each of the three libraries was panned separately against HSA (Sigma) and MSA (Sigma). In the second round of selection, phage from each of the six first round selections was panned against (i) the same antigen again (eg 1^{st} round MSA, 2^{nd} round MSA) and (ii) against the reciprocal antigen (eg 1^{st} round MSA, 2^{nd} round HSA) resulting in a total of twelve 2^{nd} round selections. In each case, after the second round of selection 48 clones were tested for binding to HSA and MSA. Soluble dAb fragments were produced as described for scFv fragments by Harrison et al, Methods Enzymol. 1996;267:83-109 and standard ELISA protocol was followed (Hoogenboom et al. (1991) Nucleic Acids Res., 19: 4133) except that 2% tween PBS was used as a blocking buffer and bound dAbs were detected with either protein L-HRP (Sigma) (for the Vκs) and protein A-HRP (Amersham Pharmacia Biotech) (for the V_{H}s).

dAbs that gave a signal above background indicating binding to MSA, HSA or both were tested in ELISA insoluble form for binding to plastic alone but all were specific for serum albumin. Clones were then sequenced (see table below) revealing that 21 unique dAb sequences had been identified. The minimum similarity (at the amino acid level) between the V_{κ} dAb clones selected was 86.25% ((69/80)×100; the result when all the diversified residues are different, eg clones 24 and 34). The minimum similarity between the V_{H} dAb clones selected was 94% ((127/136)×100).

Next, the serum albumin binding dAbs were tested for their ability to capture biotinylated antigen from solution. ELISA protocol (as above) was followed except that ELISA plate was coated with 1 μg/ml protein L (for the V_{κ} clones) and 1 μg/ml protein A (for the V_{H} clones). Soluble dAb was captured from solution as in the protocol and detection was with biotinylated MSA or HSA and streptavidin HRP. The biotinylated MSA and HSA had been prepared according to the manufacturer's instructions, with the aim of achieving an average of 2 biotins per serum albumin molecule. Twenty four clones were identified that captured biotinylated MSA from solution in the ELISA. Two of these (clones 2 and 38 below) also captured biotinylated HSA. Next, the dAbs were tested for their ability to bind MSA coated on a CM5 biacore chip. Eight clones were found that bound MSA on the biacore.

| dAb (all | | | | | Binds | |
|---|---|---|---|---|---|---|
| capture | | | | | MSA | Captures |
| biotinylated | H | | | | in | biotinylated |
| MSA) | or κ | CDR1 | CDR2 | CDR3 | biacore? | HSA? |
| Vκ library 3 | κ | XXXLX | XASXLQS | QQXXXXPXT | | |
| template | | | | | | |
| (dummy) | | | | | | |
| 2, 4, 7, 41, | κ | SSYLN | RASPLQS | QQTYSVPPT | | ✓all 4 bind |
| 38, 54 | κ | SSYLN | RASPLQS | QQTYRIPPT | | ✓both bind |
| 46, 47, 52, 56 | κ | FKSLK | NASYLQS | QQVVYWPVT | | |
| 13, 15 | κ | YYHLK | KASTLQS | QQVRKVPRT | | |
| 30, 35 | κ | RRYLK | QASVLQS | QQGLYPPIT | | |
| 19, | κ | YNWLK | RASSLQS | QQNVVIPRT | | |
| 22, | κ | LWHLR | HASLLQS | QQSAVYPKT | | |
| 23, | κ | FRYLA | HASHLQS | QQRLLYPKT | | |
| 24, | κ | FYHLA | PASKLQS | QQRARWPRT | | |
| 31, | κ | IWHLN | RASRLQS | QQVARVPRT | | |
| 33, | κ | YRYLR | KASSLQS | QQYVGYPRT | | |
| 34, | κ | LKYLK | NASHLQS | QQTTYYPIT | | |
| 53, | κ | LRYLR | KASWLQS | QQVLYYPQT | | |
| 11, | κ | LRSLK | AASRLQS | QQVVYWPAT | ✓ | |
| 12, | κ | FRHLK | AASRLQS | QQVALYPKT | ✓ | |
| 17, | κ | RKYLR | TASSLQS | QQNLFWPRT | ✓ | |
| 18, | κ | RRYLN | AASSLQS | QQMLFYPKT | ✓ | |
| 16, 21 | κ | IKHLK | GASRLQS | QQGARWPQT | ✓ | |
| 25, 26 | κ | YYHLK | KASTLQS | QQVRKVPRT | ✓ | |
| 27, | κ | YKHLK | NASHLQS | QQVGRYPKT | ✓ | |
| 55, | κ | FKSLK | NASYLQS | QQVVYWPVT | ✓ | |
| V_{H} library 1 | H | XXYXXX | XIXXXGXXTXYADSVKG | XXXX(XXXX)FDY | | |
| (and 2) | | | | | | |
| template | | | | | | |
| (dummy) | | | | | | |
| 8, 10 | H | WVYQMD | SISAFGAKTLYADSVKG | LSGKFDY | | |
| 36, | H | WSYQMT | SISSFGSSTLYADSVKG | GRDHNYSLFDY | | |

In all cases the frameworks were identical to the frameworks in the corresponding dummy sequence, with diversity in the CDRs as indicated in the table above.

Of the eight clones that bound MSA on the biacore, two clones that are highly expressed in E. coli (clones MSA16 and MSA26) were chosen for further study (see example 10). Full nucleotide and amino acid sequences for MSA16 and 26 are given in FIG. 16.

### Example 10

Determination of Affinity and Serum Half-Life in Mouse of MSA Binding dAbs MSA16 and MSA26.

As described in US20060251644, one common method for determining binding affinity is by assessing the association and dissociation rate constants using a BIAcore™ surface plasmon resonance system (BIAcore, Inc.). A biosensor chip is activated for covalent coupling of the target according to the manufacturer's (BIAcore) instructions. The target is then diluted and injected over the chip to obtain a signal in response units (RU) of immobilized material. Since the signal in RU is proportional to the mass of immobilized material, this represents a range of immobilized target densities on the matrix. Dissociation data are fit to a one-site model to obtain k_{off}±s.d. (standard deviation of measurements). Pseudo-first order rate constant (Kd's) are calculated for each association curve, and plotted as a function of protein concentration to obtain kₒₙ±s.e. (standard error of fit). Equilibrium dissociation constants for binding, Kd's, are calculated from SPR measurements as k_{off}/kₒₙ.

dAbs MSA16 and MSA26 were expressed in the periplasm of E. coli and purified using batch absorption to protein L-agarose affinity resin (Affitech, Norway) followed by elution with glycine at pH 2.2. The purified dAbs were then analysed by inhibition biacore to determine Kd. Briefly, purified MSA16 and MSA26 were tested to determine the concentration of dAb required to achieve 200RUs of response on a biacore CM5 chip coated with a high density of MSA. Once the required concentrations of dAb had been determined, MSA antigen at a range of concentrations around the expected Kd was premixed with the dAb and incubated overnight. Binding to the MSA coated biacore chip of dAb in each of the premixes was then measured at a high flow-rate of 30 μl/minute. The affinities are determined using surface plasmon resonance (SPR) and the BIAcore (Karlsson et al., 1991). The BIAcore system (Uppsala, Sweden) is a preferred method for determining binding affinity. The BIAcore system uses surface plasmon resonance (SPR, Welford K. 1991, Opt. Quant. Elect. 23:1; Morton and Myszka, 1998, Methods in Enzymology 295: 268) to monitor biomolecular interactions in real time. BIAcore analysis conveniently generates association rate constants, dissociation rate constants, equilibrium dissociation constants, and affinity constants. The resulting curves were used to create Klotz plots, (Klotz, I. M. (1982) Science 217:1247-1249 and Klotz, I. M. (1983) J. Trends in Pharmacol. Sci. 4:253-255) which gave an estimated Kd of 200 nM for MSA16 and 70 nM for MSA 26 (FIGS. 17A & B).

Next, clones MSA16 and MSA26 were cloned into an expression vector with the HA tag (nucleic acid sequence: TATCCTTATGATGTTCCTGATTATGCA and amino acid sequence: YPYDVPDYA) and 2-10 mg quantities were expressed in E. coli and purified from the supernatant with protein L-agarose affinity resin (Affitech, Norway) and eluted with glycine at pH2.2. Serum half life of the dAbs was determined in mouse. MSA26 and MSA16 were dosed as single i.v. injections at approx 1.5 mg/kg into CD1 mice. Analysis of serum levels was by goat anti-HA (Abeam, UK) capture and protein L-HRP (invitrogen) detection ELISA which was blocked with 4% Marvel. Washing was with 0.05% tween PBS. Standard curves of known concentrations of dAb were set up in the presence of 1×mouse serum to ensure comparability with the test samples. Modelling with a 2 compartment model showed MSA-26 had a t½α of 0.16 hr, a t½β of 14.5 hr and an area under the curve (AUC) of 465 hr.mg/ml (data not shown) and MSA-16 had a t½α of 0.98 hr, a t½β of 36.5 hr and an AUC of 913 hr.mg/ml (FIG. 18). Both anti-MSA clones had considerably lengthened half life compared with HEL4 (an anti-hen egg white lysozyme dAb) which had a t½α of 0.06 hr, and a t½β of 0.34 hr.

### Example 11

Creation of V_{H}-V_{H} and Vκ-Vκ Dual Specific Fab Like Fragments

This example describes a method for making V_{H}-V_{H} and Vκ-Vκ dual specifics as Fab like fragments. Before constructing each of the Fab like fragments described, dAbs that bind to targets of choice were first selected from dAb libraries similar to those described in example 9. A V_{H} dAb, HEL4, that binds to hen egg lysozyme (Sigma) was isolated and a second V_{H} dAb (TAR2h-5) that binds to TNFα receptor (R and D systems) was also isolated. The sequences of these are given in the sequence listing. A Vκ dAb that binds TNFα (TAR1-5-19) was isolated by selection and affinity maturation and the sequence is also set forth in the sequence listing. A second Vκ dAb (MSA 26) described in example 9 whose sequence is in FIG. 17B was also used in these experiments.

DNA from expression vectors containing the four dAbs described above was digested with enzymes SalI and NotI to excise the DNA coding for the dAb. A band of the expected size (300-400 bp) was purified by running the digest on an agarose gel and excising the band, followed by gel purification using the Qiagen gel purification kit (Qiagen, UK). The DNA coding for the dAbs was then inserted into either the C_{H} or Cκ vectors (FIGS. 8 and 9) as indicated in the table below.

| | | dAb V_{H} or | Inserted into | tag (C | Antibiotic |
|---|---|---|---|---|---|
| dAb | Target antigen | dAb Vκ | vector | terminal) | resistance |
| HEL4 | Hen egg lysozyme | V_{H} | C_{H} | myc | Chloramphenicol |
| TAR2-5 | TNF receptor | V_{H} | Cκ | flag | Ampicillin |
| TAR1-5-19 | TNF α | Vκ | C_{H} | myc | Chloramphenicol |
| MSA 26 | Mouse serum albumin | Vκ | Cκ | flag | Ampicillin |

The V_{H} C_{H} and V_{H} Cκ constructs were cotransformed into HB2151 cells. Separately, the Vκ C_{H} and Vκ Cκ constructs were cotransformed into HB2151 cells. Cultures of each of the cotransformed cell lines were grown overnight (in 2xTy containing 5% glucose, 10 μg/ml chloramphenicol and 100 μg/ml ampicillin to maintain antibiotic selection for both C_{H} and Cκ plasmids). The overnight cultures were used to inoculate fresh media (2xTy, 10 μg/ml chloramphenicol and 100 μg/ml ampicillin) and grown to OD 0.7-0.9 before induction by the addition of IPTG to express their C_{H} and Cκ constructs. Expressed Fab like fragment was then purified from the periplasm by protein A purification (for the contransformed V_{H} C_{H} and V_{H} Cκ) and MSA affinity resin purification (for the contransformed Vκ C_{H} and Vκ Cκ).

V_{H}-V_{H} Dual Specific

Expression of the V_{H} C_{H} and V_{H} Cκ dual specific was tested by running the protein on a gel. The gel was blotted and a band the expected size for the Fab fragment could be detected on the Western blot via both the myc tag and the flag tag, indicating that both the V_{H} C_{H} and V_{H} Cκ parts of the Fab like fragment were present. Next, in order to determine whether the two halves of the dual specific were present in the same Fab-like fragment, an ELISA plate was coated overnight at 4° C. with 100 μl per well of hen egg lysozyme (HEL) at 3 mg/ml in sodium bicarbonate buffer. The plate was then blocked (as described in example 1) with 2% tween PBS followed by incubation with the V_{H} C_{H}/V_{H} Cκ dual specific Fab like fragment. Detection of binding of the dual specific to the HEL was via the non cognate chain using 9e10 (a monoclonal antibody that binds the myc tag, Roche) and anti mouse IgG-HRP (Amersham Pharmacia Biotech). The signal for the V_{H} C_{H}/V_{H} Cκ dual specific Fab like fragment was 0.154 compared to a background signal of 0.069 for the V_{H} Cκ chain expressed alone. This demonstrates that the Fab like fragment has binding specificity for target antigen.

V_{K}-V_{K} Dual Specific

After purifying the contransformed Vκ C_{H} and Vκ Cκ dual specific Fab like fragment on an MSA affinity resin, the resulting protein was used to probe an ELISA plate coated with 1 μg/ml TNFα and an ELISA plate coated with 10 μg/ml MSA. As predicted, there was signal above background when detected with protein L-HRP on both ELISA plates (data not shown). This indicated that the fraction of protein able to bind to MSA (and therefore purified on the MSA affinity column) was also able to bind TNFα in a subsequent ELISA, confirming the dual specificity of the antibody fragment. This fraction of protein was then used for two subsequent experiments. Firstly, an ELISA plate coated with 1 μg/ml TNFα was probed with dual specific Vκ C_{H} and Vκ Cκ Fab like fragment and also with a control TNFα binding dAb at a concentration calculated to give a similar signal on the ELISA. Both the dual specific and control dAb were used to probe the ELISA plate in the presence and in the absence of 2 mg/ml MSA. The signal in the dual specific well was reduced by more than 50% but the signal in the dAb well was not reduced at all (see FIG. 19a). The same protein was also put into the receptor assay with and without MSA and competition by MSA was also shown (see FIG. 19c). This demonstrates that binding of MSA to the dual specific is competitive with binding to TNFα.

### Example 12

Creation of a Vκ-Vκ Dual Specific Cys Bonded Dual Specific with Specificity for Mouse Serum Albumin and TNFα

This example describes a method for making a dual specific antibody fragment specific for both mouse serum albumin and TNFα by chemical coupling via a disulphide bond. Both MSA16 (from example 1) and TAR1-5-19 dAbs were recloned into a pET based vector with a C terminal cysteine and no tags. The two dAbs were expressed at 4-10 mg levels and purified from the supernatant using protein L-agarose affinity resin (Affitiech, Norway). The cysteine tagged dAbs were then reduced with dithiothreitol. The TAR1-5-19 dAb was then coupled with dithiodipyridine to block reformation of disulphide bonds resulting in the formation of PEP 1-5-19 homodimers. The two different dAbs were then mixed at pH 6.5 to promote disulphide bond formation and the generation of TAR1-5-19, MSA16 cys bonded heterodimers. This method for producing conjugates of two unlike proteins was originally described by King et al. (King T P, Li Y Kochoumian L Biochemistry. 1978 vol 17:1499-506 Preparation of protein conjugates via intermolecular disulfide bond formation.) Heterodimers were separated from monomeric species by cation exchange. Separation was confirmed by the presence of a band of the expected size on a SDS gel. The resulting heterodimeric species was tested in the TNF receptor assay and found to have an IC50 for neutralising TNF of approximately 18 nM. Next, the receptor assay was repeated with a constant concentration of heterodimer (18nM) and a dilution series of MSA and HSA. The presence of HSA at a range of concentrations (up to 2 mg/ml) did not cause a reduction in the ability of the dimer to inhibit TNFα. However, the addition of MSA caused a dose dependant reduction in the ability of the dimer to inhibit TNFα (FIG. 20). This demonstrates that MSA and TNFα compete for binding to the cys bonded TAR1-5-19, MSA16 dimer.

### Example 13

### Cloning and Expression of the TAR1/TAR2 Dual Specific Fab

TAR1-5-19 V_{κ} dAb (specific to human TNF alpha) was cloned into pDOM3 CK Amp vector (FIG. 21) as a SalI/NotI fragment. TAR2h-10-27 VH dAb (specific to human TNFRI) was cloned into pDOM3 CH Chlor vector (FIG. 21) as a SalI/NotI fragment.

The two vectors with cloned in dAbs were used to co-transform competent HB2151 cells. Amp/Chlor resistant clones (containing both plasmids) were used to make a large scale (101) fermentor prep of the Fab.

The produced Fab was isolated from the culture supernatant (after 3 hours induction at 25 C) using sequential Protein A/Protein L purification. The yield of the Fab was 1.5 mg.

### Example 14

### Analysis of Fab Properties in ELISA

a) Binding of the Fab to TAR1 and TAR2

Binding of the TAR1/TAR2 Fab to TNF and TNFRI was tested in ELISA. A 96 well plate was coated with 100 ul of TNF and TNFRI at 1 ug/ml concentration in PBS overnight at 4 C. 50 ul (3 uM) of Fab was then added to the wells and bound Fab was detected via non-cognate chain, ie using Protein A-HRP on TNF coated wells and Protein L-HRP on TNFRI coated wells. ELISA demonstrated the ability of the Fab to bind both antigens (FIG. 22).

b) Sandwich ELISA

To test the ability of the TAR1/TAR2 Fab to bind both antigens simultaneously (open/closed conformation?) a sandwich ELISA was performed. Here a 96 well plate was coated with mutant TNF (that does not bind to TNFRI, but does bind to PEP1-5-19, data no shown; mutant TNF contains a single point mutation (N141Y) which renders it incapable of binding to TNFRI (Yamadishi et al., 1990, Protein Eng., 3, 713-9)) at 1 ug/ml concentration in PBS overnight at 4 C. 50 ul of Fab (0.5 uM) was then added. This was followed by addition of TNFRI-Fc fusion protein (R&D Systems) and detection with Anti-Fc-HRP. The same sandwich ELISA was performed using a control Fab containing TAR1/Ck chain and an irrelevant VH fused to the CH chain. ELISA results demonstrated the ability of the Fab to engage both antigens (TNF and TNFRI) simultaneously, suggesting an open conformation of the molecule (FIG. 23).

c) Competition ELISA

To test the ability of the TAR1/TAR2 Fab to bind both antigens simultaneously two competition ELISAs were performed.

A 96 well plate was coated with 100 ul of TNFRI at 1 ug/ml concentration in PBS overnight at 4 C. A dilution of Fab was chosen such that OD450 of 0.3 was achieved upon detection with Protein L-HRP. This concentration was 6 nM. The Fab was pre-incubated for an hour at room temperature with increasing concentrations of mutant TNF (up to 160× molar excess). As a negative control Fab was subjected to the same incubation with BSA. Following these incubations the mixtures were then put onto TNFRI coated ELISA plate and incubated for another hour. Bound TAR1/TAR2 Fab was detected using Protein L-HRP. ELISA demonstrated that TAR1/TAR2 Fab binding to TNFRI was not affected by competing antigen (FIG. 24).

A 96 well plate was coated with 100 ul of mutant TNF at 1 ug/ml concentration in PBS overnight at 4 C. A dilution of Fab was chosen such that OD450 of 0.3 was achieved upon detection with 9E10 (Sigma) followed by anti mo-HRP (Sigma). This concentration was 25 nM. The Fab was pre-incubated for an hour at room temperature with increasing concentrations of soluble TNFRI (up to 10× molar excess). As a negative control Fab was subjected to the same incubation with BSA. Following these incubations the mixtures were then put onto mutant TNF coated ELISA plate and incubated for another hour. Bound TAR1/TAR2 Fab was detected using 9E10 followed by anti mo-HRP. ELISA demonstrated that TAR1/TAR2 Fab binding to mutant TNF was not affected by competing antigen (FIG. 24).

### Example 15

### Analysis of Fab Properties in Cell Assays

To check the degree of functionality of each dAb in a TAR1/TAR2 Fab, the performance of the dual specific molecule was tested in the following cell assays:

Human TNF Cytotoxicity on Murine Cells.

This assay tests the activity of TAR1 Dab, as TAR2 Dab cannot bind to murine TNF receptor expressed on the surface of the cells. TAR1-5-19 Dab and TAR2h-10-27 dAbs as well as TAR1-5-19+TAR2h-10-27 dAb mixture were used as controls in this assay. The results demonstrate that TAR1-5-19 Dab in a Fab behaves as well as a monomeric TAR1-5-19 dAb (FIG. 25).

Murine TNF Cytotoxicity Assay on Murine Cells with Human Soluble TNF Receptor.

This assay tests the activity of TAR2h-10-27 (in this assay binding to soluble human TNFRI). TAR1-5-19 and TAR2h-10-27 dAbs as well as TAR1-5-19+TAR2h-10-27 dAb mixture were used as controls in this assay. The results demonstrate that TAR2h-10-27 in a Fab behaves as well as a monomeric TAR2h-10-27 dAb (FIG. 25).

Murine TNF Induced IL-8 Secretion on Human Cells.

This assay tests the activity of TAR2h-10-27 Dab (in this assay binding to membrane bound human TNFRI). TAR1-5-19 and TAR2h-10-27 dAbs as well as TAR1+TAR2 dAb mixture were used as controls in this assay. The results demonstrate that TAR2h-10-27 in a Fab behaves as well as a monomeric TAR2h-10-27 dAb (FIG. 25).

Human TNF Induced IL-8 Secretion on Human Cells.

This assay tests the activity of both TAR1-5-19 and TAR2h-10-27 Dabs. TAR1-5-19 Daband as controls in this assay. The results demonstrate that Fab has a similar effect to the TAR2h-10-27 dAb and TAR1-5-19+TAR2h-10-27 dAb mixture (FIG. 25).

Murine TNF Cytotoxicity on Murine Cells with Soluble Human TNFRI and Increasing Concentrations of Mutant TNF (Competition on Cells).

This assay was performed to test whether increasing concentration of mutant TNF (binding to TAR 1-5-19 Dab) will compromise binding of TAR2h-10-27 Dab to TNFRI in solution. The results of the assay indicate that that is not the case, thus the Fab is able to engage two antigens simultaneously (FIG. 26).

The assays described above demonstrate that each dAb in a Fab molecule functions as well as a monomeric dAb.

### Example 16

### Construction of IgG Vectors

pcDNA3.1(+) and pcDNA3.1/Zeo(+) backbones (Invitrogen) were used for cloning IgG1 heavy chain constant region and light chain kappa constant region, respectively. The overview of the vectors is shown in FIG. 27.

Leaders:

Two alternative types of leaders were used to facilitate secretion of the expressed protein:

CD33 leader

IgG K-chain leader

The leaders were assembled by the annealing of the two complementary oligos (Table 5) and were cloned into pcDNA3.1(+) and pcDNA3.1/Zeo(+) as NheI/HindIII fragments (FIG. 27).

IgG1 Heavy Chain Cloning:

CH1 domain was PCR amplified from the CH vector (as described in WO 03/002609) using primers shown in Table 5.

Hinge region, CH2 and CH3 domains were PCR amplified from plgplus vector (Novagen) using primers shown in Table 5.

The two products were then PCR assembled to create an IgG1 heavy chain constant region which was cloned into pcDNA3.1(+) as a NotI/XhoI fragment (FIG. 27).

Kappa Light Chain Cloning:

CK domain was PCR amplified from the CK vector (see WO 03/002609) using primers shown in Table 5.

It was then cloned into pcDNA3.1/Zeo (+) as a NotI/XhoI fragment (FIG. 27).

### Example 17

### Cloning of TAR1-5-19 and TAR2h-10-27 dAbs into IgG Vectors and Production of IgG

TAR1-5-19 VK dAb (specific to human TNF alpha) was cloned into IgG kappa vectors (with CD33 and IgK leaders) as a HindIII/NotI fragment (FIG. 27).

TAR2h-10-27 VH dAb (specific to human TNFRI) was cloned into IgG heavy chain vectors (with CD33 and IgK leaders) as a HindIII/NotI fragment (FIG. 27).

Heavy and light chain plasmids were then co-transfected into COS7 cells and IgG was expressed transiently for five days. The produced IgG was purified using streamline Protein A. Expression level—250 ng/ml. CD33 and IgG K leaders gave the same level of expression.

Purified IgG was checked on a reducing and non-reducing SDS gel (produced bands of expected size) (data not shown).

### Example 18

### Analysis of IgG Properties in ELISA

a) Binding of the IgG to TNF and TNFRI

Binding of the TAR1/TAR2 IgG to TNF and TNFRI was tested in ELISA. A 96 well plate was coated with 100 ul of TNF and TNFRI at 1 ug/ml concentration in PBS overnight at 4 C. 50 ul (200 nM) of IgG was then added to the wells and bound IgG was detected via anti-Fc-HRP. ELISA demonstrated the ability of the IgG to bind both antigens (FIG. 28).

### Example 19

### Analysis of IgG Properties in Cell Assays

To check the degree of functionality of each dAb in a TAR1/TAR2 IgG, the performance of the dual specific molecule was tested in the following cell assays:

Human TNF Cytotoxicity on Murine Cells.

This assay tests the activity of TAR1-5-19 Dab, as TAR2h-10-27 Dab cannot bind to murine TNF receptor expressed on the surface of the cells. TAR1-5-19 and TAR2h-10-27 dAbs as well as TAR1-5-19+TAR2h-10-27 dAb mixture were used as controls in this assay. The results demonstrate that TAR1-5-19 in the IgG behaves better than monomeric TAR1-5-19 dAb, which indicates that IgG is able to simultaneously engage two molecules of TNF (ND50 of the dimeric molecule) (FIG. 29).

Murine TNF Cytotoxicity Assay on Murine Cells with Human Soluble TNF Receptor.

This assay tests the activity of TAR2h-10-27 (in this assay binding to soluble human TNFRI). TAR1-5-19 and TAR2h-10-27 dAbs as well as TAR1-5-19+TAR2h-10-27 dAb mixture were used as controls in this assay. The results demonstrate that TAR2h-10-27 in IgG behaves as well as a monomeric TAR2h-10-27 dAb (FIG. 29).

Murine TNF Induced IL-8 Secretion on Human Cells.

This assay tests the activity of TAR2h-10-27 (in this assay binding to membrane bound human TNFRI). TAR1-5-19 and TAR2h-10-27 dAbs as well as TAR1-5-19+TAR2h-10-27 dAb mixture were used as controls in this assay. The results demonstrate that IgG is able to engage two molecules of TNFRI on the surface of the cell (agonistic activity) (FIG. 29). This assay was also repeated with no human TNF present. The results demonstrate that the IgG induces IL-8 release on human cells up to a concentration of 30 nM after which the agonistic activity goes down (FIG. 30).

Human TNF Induced IL-8 Secretion on Human Cells.

This assay tests the activity of both TAR1-5-19 and TAR2h-10-27. TAR1-5-19 and TAR2h-10-27 dAbs as well as TAR1-5-19+TAR2h-10-27 dAb mixture were used as controls in this assay. The results demonstrate that IgG has a similar effect to the TARh-10-27 dAb and TAR1-5-19 +TAR2h-10-27 dAb mixture (FIG. 29). This assay was also repeated with no murine TNF present. The results demonstrate that the IgG induces IL-8 release on human cells up to a concentration of 30 nM after which the agonistic activity goes down (FIG. 30).

TABLE 5
Primers/Oligos

CkbckNot
5′ AAGGAAAAAAGCGGCCGCAACTGTGGCTGCACCATC 3′
CkforXho
5′ CCGCTCGAGTCAACACTCTCCCCTGTTGAAGCTCTTTGTG 3′
Ch1bckNot
5′ AAGGAAAAAAGCGGCCGCCTCCACCAAGGGCCCATCGGTC 3′
Ch1for
5′ GTGAGGTTTGTCACAAGATTTGGGCTCAACTTTCTTGTCCACC 3′
Fcbck
5′ CCCAAATCTTGTGACAAACCTCAC 3′
FcforXho
5′ CCGCTCGAGTCATTTACCCGGAGACAGGGAG 3′
LEADER CD33:
Leacd1
5′P CTAGCCACCATGCCGCTGCTGCTACTGCTGCCACTGCTGTGGGCAG
GAGCACTGGCTATGGATA 3′
Leacd2
5′P AGCTTATCCATAGCCAGTGCTCCTGCCCACAGCAGTGGCAGCAGTA
GCAGCAGCGGCATGGTGG 3′
LEADER IGGK:
Leak1
5′P
CTAGCCACCATGGAGACAGACACACTCCTGCTATGGGTACTGCTGCTCTG
GGTTCCAGGTTCCACTGGTGACA 3′
Leak2
5′P
AGCTTGTCACCAGTGGAACCTGGAACCCAGAGCAGCAGTACCCATAGCAG
GAGTGTGTCTGTCTCCATGGTGG 3′
SEQBACK
5′ TAATACGACTCACTATAGGG 3′
SEQFOR
5′ TAGAAGGCACAGTCGAGG 3′

Data Summary

A summary of data obtained in the experiments set forth in the foregoing examples is set forth in Annex 4.

### Example 20

### Summary of Nucleic Acid and Polypeptide Sequences for Anti-TNF-α dAbs

Throughout the course of studies regarding the anti-TNF-α dAbs described herein, a number of different dAbs have been identified that bind human and/or mouse TNF-α. Sequences and further information are provided herein below.

Clones that Bind Mouse TNF-α:

The nucleotide and amino acid sequences for four anti-mouse TNF-α dAbs are provided below. Two of these (TAR1-2m-9 and TAR1-2m-30) inhibit the activity of mouse TNF-α, and two bind but do not inhibit (TAR1-2m-1 and TAR1-2m-2).

TAR1-2m-9:

TAR-2m-9 is a Vk clone, with an 1050 of 6 μM and an ND50 of 5 μM. The IC50 and ND50 are not improved upon Protein L cross-linking. This clone has no effect against human TNF-α (species cross-reactivity has been assessed in cell assays at two concentrations), but has similar neutralizing activity against rat TNF-α.

| Amino acid sequence (CDR3 is in BOLD) (SEQ ID NO: 93): | |
|---|---|
| DIQMTQSPSSLSASVGDRVTITCRASQPIGSFLWWYQQKPGKAPKLLIY | |
| YSSYLQSGVPSRFSGSGSGTDFTLTISSLQPEDFATYYCQQYRWHPN | |
| TFGQGTKVEIKR | |
| Nucleotide sequence (SEQ ID NO: 94): | |
| 1 | gacatccagatgacccagtctccatcctccctgtctgcatctgtaggagaccgtgtcacc |
| 61 | atcacttgccgggcaagtcagcctattgggagttttttatggtggtaccagcagaaacca |
| 121 | gggaaagcccctaaactcctgatctattatagttcctatttgcaaagtggggtcccatca |
| 181 | cgtttcagtggcagtggatctgggacagatttcactctcaccatcagcagtctgcaacct |
| 241 | gaagattttgctacgtactactgtcaacagtatcgttggcatcctaataccttcggccaa |
| 301 | gggaccaaggtggaaatcaaacgg |

TAR1-2m-30:

TAR1-2m-30 is a Vk clone, with an ND50 of 10 μM. ND50 is not improved upon Protein L cross-linking. This clone has no effect against human TNF-α (species cross-reactivity has been assessed in cell assays at two concentrations), and is slightly less effective against rat TNF when compared to mouse.

| Amino acid sequence (CDR3 is in BOLD) (SEQ ID NO: 95): | |
|---|---|
| DIQMTQSPSSLSASVGDRVTITCRASQSIYSWLNWYQQKPGKAPKLLIY | |
| RASHLQSGVPSRFSGSGSGTDFTLTISSLQPEDFATYYCQQIWNMPF | |
| TFGQGTKVEIKR | |
| Nucleotide sequence (SEQ ID NO: 96): | |
| 1 | gacatccagatgacccagtctccatcctccctgtctgcatctgtaggagaccgtgtcacc |
| 61 | atcacttgccgggcaagtcagtcgatttatagttggttaaattggtaccagcagaaacca |
| 121 | gggaaagcccctaagctcctgatctatagggcgtcccatttgcaaagtggggtcccatca |
| 181 | cgtttcagtggcagtggatctgggacagatttcactctcaccatcagcagtctgcaacct |
| 241 | gaagattttgctacgtactactgtcaacagatttggaatatgccttttacgttcggccaa |
| 301 | gggaccaaggtggaaatcaaacgg |

TAR1-2m-1:

This clone binds mouse TNF-α but does not inhibit receptor binding activity.

| Amino acid sequence (SEQ ID NO: 97): | |
|---|---|
| DIQMTQSPSSLSASVGDRVTITCRASQPIGYDLFWYQQKPGKAPKLLIY | |
| RGSVLQSGVPSRFSGSGSGTDFTLTISSLQPEDFATYYCQQRWRWPFT | |
| FGQGTKVEIKR | |
| Nucleotide sequence (SEQ ID NO: 98): | |
| 1 | gacatccagatgacccagtctccatcctccctgtctgcatctgtaggagaccgtgtcacc |
| 61 | atcacttgccgggcaagtcagcctattggttatgatttattttggtaccagcagaaacca |
| 121 | gggaaagcccctaagctcctgatctatcggggttccgtgttgcaaagtggggtcccatca |
| 181 | cgtttcagtggcagtggatctgggacagatttcactctcaccatcagcagtctgcaacct |
| 241 | gaagattttgctacgtactactgtcaacagcggtggcgttggccttttacgttcggccaa |
| 301 | ggcaccaaggtggaaatcaaacgg |

TAR1-2m-2:

This clone binds mouse TNF-α but does not inhibit receptor binding activity.

| Amino acid sequence (SEQ ID NO: 99): | |
|---|---|
| DIQMTQSPSSLSASVGDRVTITCRASLPIGRDLWWYQQKPGKAPKLLIY | |
| RGSFLQSGVPSRFSGSGSGTDFTLTISSLQPEDFATYYCQQRWYYPHTF | |
| GQGTKVEIKR | |
| Nucleotide sequence (SEQ ID NO: 100): | |
| 1 | gacatccagatgacccagtctccatcctccctgtctgcatctgtaggagaccgtgtcacc |
| 61 | atcacttgccgggcaagtctgcctattggtcgtgatttatggtggtatcagcagaaacca |
| 121 | gggaaagcccctaagctcctgatctatcgggggtcctttttgcaaagtggggtcccatca |
| 181 | cgtttcagtggcagtggatctgggacagatttcactctcaccatcagcagtctgcaacct |
| 241 | gaagattttgctacgtactactgtcaacagaggtggtattatcctcatacgttcggccaa |
| 301 | gggaccaaggtggaaatcaaacgg |

dAb Clones that Bind Human TNF-α

The following is a listing of the nucleotide sequences of dAbs identified for binding human TNF-α. Corresponding amino acid sequences are provided in FIG. 34.

TAR1-5

(SEQ ID NO: 101)

GACATCCAGATGACCCAGTCTCCATCCTCCCTGTCTGCATCTGTAGGAGA
CCGTGTCACCATCACTTGCCGGGCAAGTCAGAGCATTTTTATGAATTTAT
TGTGGTACCAGCAGAAACCAGGGAAAGCCCCTAAGCTCCTGATCTATAAT
GCATCCGTGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGC
TAR1-27

(SEQ ID NO: 102)

GACATCCAGATGACCCAGTCTCCATCCTCCCTGTCTGCATCTGTAGGAGA
CCGTGTCACCATCACTTGCCGGGCAAGTCAGAGCATTTGGACGAAGTTAC
ATTGGTACCAGCAGAAACCAGGGAAAGCCCCTAAGCTCCTGATCTATATG
GCATCCAGTTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGTGGTTTAGTAATCCTAGTACGTTCGGCCAA
GGGACCAAGGTGGAAATCAAACG
TAR1-261

(SEQ ID NO: 103)

GACATCCAGATGACCCAGTCTCCATCCTCCCTGTCTGCATCTGTAGGAGA
CCGTGTCACCATCACTTGCCGGGCAAGTCAGAGCATTGAGCATTATTTAT
GGTGGTACCAGCAGAAACCAGGGAAAGCCCCTAAGCTCCTGATCTATGCT
GCATCCTATTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGAGTTTGGCGTGTCCTCCTACGTTCGGCCAA
GGGACCAAGGTGGAAATCAAACGG
TAR1-398

(SEQ ID NO: 104)

GACATCCAGATGACCCAGTCTCCATCCTCCCTGTCTGCATCTGTAGGAGA
CCGTGTCACCATCACTTGCCGGGCAAGTCAGAGCATTTATGGTCATTTAT
TGTGGTACCAGCAGAAACCAGGGAAAGCCCCTAAGCTCCTGATCTATGCT
GCATCCAGTTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGCCTTTGGTGCGGCCTTTTACGTTCGGCCAA
GGGACCAAGGTGGAAATCAAACGG
TAR1-701

(SEQ ID NO: 105)

GACATCCAGATGACCCAGTCTCCATCCTCCCTGTCTGCATCTGTAGGAGA
CCGTGTCACCATCACTTGCCGGGCAAGTCAGAGCATTGCTAAGTTGTTAT
ATTGGTACCAGCAGAAACCAGGGAAAGCCCCTAAGCTCCTGATCTATGAT
GCATCCTCTTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGTGGTGGGGGTATCCTGGTACGTTCGGCCAA
GGGACCAAGGTGGAAATCAAACGG
TAR1-5-2

(SEQ ID NO: 106)

GACATCCAGATGACCCAGTCTCCATCCTCCCTGTCTGCATCTGTAGGAGA
CCGTGTCACCATCACTTGCCGGGCAAGTCAGAGCATTTTTCCTGCTTTAC
TTTGGTACCAGCAGAAACCAGGGAAAGCCCCTAAGCTCCTGATCTATCAT
GCATCCAGTTTGCAAAGTGGGGTCCCATCACGTTTCAGTGGCAGTGGATC
TGGGACAGATTTCACTCTCACCATCAGCAGTCTGCAACCTGAAGATATTG
CTACGTACTACTGTCAACAGGTTGTGTGGCGTCCTTTTACGTTCGGCCAA
GGGACCAAGGTGGAAATCAAACGG
TAR1-5-3

(SEQ ID NO: 107)

GACATCCAGATGACCCAGTCTCCATCCTCCCTGTCTGCATCTGTAGGAGA
CCGTGTCACCATCACTTGCCGGGCAAGTCAGAGCATTGATAATGCGTTAC
ATTGGTACCAGCAGAAACCAGGGAAAGCCCCTAAGCTCCTGATCTATCAG
GCATCCATTTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-4

(SEQ ID NO: 108)

GACATCCAGATGACCCAGTCTCCATCCTCCCTGTCTGCATCTGTAGGAGA
CCGTGTCACCATCACTTGCCGGGCAAGTCAGAGCATTTTTATGAATTTAT
TGTGGTACCAGCAGAAACCAGGGAAAGCCCCTAAGCTCCTGATCTATAAT
GCATCCGTGTTGCAAAGTGGGGTCCCATCACGTTTCAGTGGCAGTGGATC
TGGGACAGGTTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-7

(SEQ ID NO: 109)

GACATCCAGATGACCCAGTCTCCATCCTCCCTGTCTGCATCTGTAGGAGA
CCGTGTCACCATCACTTGCCGGGCAAGTCAGAGCATTTTGAATTCTTTAC
ATTGGTACCAGCAGAAACCAGGGAAAGCCCCTAAGCTCCTGATCTATCAT
GCATCCACTTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-8

(SEQ ID NO: 110)

GACATCCAGATGACCCAGTCTCCATCCTCCCTGTCTGCATCTGTAGGAGA
CCGTGTCACCATCACTTGCCGGGCAAGTCAGAGCATTTTGAATTCTTTAC
ATTGGTACCAGCAGAAACCAGGGAAAGCCCCTAAGCTCCTGATCTATCAT
GCATCCACTTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-10

(SEQ ID NO: 111)

GACATCCAGATGACCCAGTCTCCATCCTCCCTGTCTGCATCTGTAGGAGA
CCGTGTCACCATCACTTGCCGGGCAAGTCAGAGCATTGATAATTATTTAC
ATTGGTACCAGCAGAAACCAGGGAAAGCCCCTAAGCTCCTGATCTATTCT
GCATCCCATTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-11

(SEQ ID NO: 112)

GACATCCAGATGACCCAGTCTCCATCCTCCCTGTCTGCATCTGTAGGAGA
CCGTGTCACCATCACTTGCCGGGCAAGTCAGAGCATTAATGAGTATTTAC
ATTGGTACCAGCAGAAACCAGGGAAAGCCCCTAAGCTCCTGATCTATTCT
GCATCCGTGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-12

(SEQ ID NO: 113)

GACATCCAGATGACCCAGTCTCCATCCTCCCTGTCTGCATCTGTAGGAGA
CCGTGTCACCATCACTTGCCGGGCAAGTCAGAGCATTAATTATGCTTTAC
ATTGGTACCAGCAGAAACCAGGGAAAGCCCCTAAGCTCCTGATCTATCAG
GCATCCATTTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-13

(SEQ ID NO: 114)

GACATCCAGATGACCCAGTCTCCATCCTCCCTGTCTGCATCTGTAGGAGA
CCGTGTCACCATCACTTGCCGGGCAAGTCAGAGCATTGATAGTTTTTTAC
ATTGGTACCAGCAGAAACCAGGGAAAGCCCCTAAGCTCCTGATCTATAGT
GCATCCGAGTTGCAAAGTGGGGTCCCATCACGTTTCAGTGGCAGTGGATC
TGGGACAGATTTCACTCTCACCATCAGCAGTCTGCATCCTGAAGATTTTG
CTACGTACTACTGTCAACAGGTTGTGTGGCGTCCTTTTACGTTCGGCCAA
GGGACCAAGGTGGAAATCAAACGG
TAR1-5-19

(SEQ ID NO: 115)

GACATCCAGATGACCCAGTCTCCATCCTCTCTGTCTGCATCTGTAGGAGA
CCGTGTCACCATCACTTGCCGGGCAAGTCAGAGCATTGATAGTTATTTAC
ATTGGTACCAGCAGAAACCAGGGAAAGCCCCTAAGCTCCTGATCTATAGT
GCATCCGAGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-20

(SEQ ID NO: 116)

GACATCCAGATGACCCAGTCTCCATCCTCCCTGTCTGCATCTGTAGGAGA
CCGTGTCACCATCACTTGCCGGGCAAGTCAGAGCATTGATCAGTATTTAC
ATTGGTACCAGCAGAAACCAGGGAAAGCCCCTAAGCTCCTGATCTATGGT
GCATCCAATTTGCAAAGTGAGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-21

(SEQ ID NO: 117)

GACATCCAGATGACCCAGTCTCCATCCTCCCTGTCTGCATCTGTAGGAGA
CCGTGTCACCATCACTTGCCGGGCAAGTCAGAGCATTGATAGTTTTTTAC
ATTGGTACCAGCAGAAACCAGGGAAAGCCCCTAAGCTCCTGATCTATAGT
GCATCCGAGTTGCAAAGTGGGGTCCCATCACGTTTCAGTGGCAGTGGATC
TGGGACAGATTTCACTCTCACCATCAGCAGTCTGCATCCTGAAGATTTTG
CTACGTACTACTGTCAACAGGTTGTGTGGCGTCCTTTTACGTTCGGCCAA
GGGACCAAGGTGGAAATCAAACGG
TAR1-5-22

(SEQ ID NO: 118)

GACATCCAGATGACCCAGTCTCCATCCTCCCTGTCTGCATCTGTAGGAGA
CCGTGTCACCATCACTTGCCGGGCAAGTCAGAGCATTGATTCTTATTTAC
ATTGGTACCAGCAGAAACCAGGGAAAGCCCCTAAGCTCCTGATCTATAGT
GCATCCCTGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-23

(SEQ ID NO: 119)

GACATCCAGATGACCCAGTCTCCATCCTCCCTGTCTGCATCTGTAGGAGA
CCGTGTCACCATCACTTGCCGGGCAAGTCAGAGCATTGATCAGTATTTAC
ATTGGTACCAGCAGAAACCAGGGAAAGCCCCTAAGCTCCTGATCTATTCT
GCATCCCTTTTGCAAAGTGGGGTCCCATCACGTTTCAGTGGCAGTGGATC
TGGGACAGATTTCACTCTCACCATCAGCAGTCTGCAACCTGAAGATTTTG
CTACATACTACTGTCAACAGGTTGTGTGGCGTCCTTTTACGTTCGGCCAA
GGGACCAAGGTGGAAATCAAACGG
TAR1-5-24

(SEQ ID NO: 120)

GACATCCAGATGACCCAGTCTCCATCCTCCCTGTCTGCATCTGTAGGAGA
CCGTGTCAGCATCACTTGCCGGGCAAGTCAAAGCATTGATGAGTTTTTAC
ATTGGTACCAGCAGAAACCAGGGAAAGCCCCTAAGCTCCTGATCTATTGT
GCATCCCAGTTGCAAAGTGGGGTCCCATCACGTTTCAGTGGCAGTGGATC
TGGGACAGATTTCACTCTCACCATCAGCAGTCTACATCCTGAAGATTTTG
CTACGTACTACTGTCAACAGGTTGTGTGGCGTCCTTTTACGTTCGGCCAA
GGGACCAAGGTGGAAATCAAACGG
TAR1-5-25

(SEQ ID NO: 121)

GACATCCAGATGACCCAGTCTCCATCCTCCCTGTCTGCATCTGTAGGAGA
CCGTGTCACCATCACTTGCCGGGCAAGTCAGAGCATTGATGCGTATTTAC
ATTGGTACCAGCAGAAACCAGGGAAAGCCCCTAAGCTCCTGATCTATTCT
GCATCCCTGTTGCAAAGTGGGGTCCCATGACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-26

(SEQ ID NO: 122)

GACATCCAGATGACCCAGTCTCCATCCTCCCTGTCTGCATCTGTAGGAGA
CCGTGTCACCATCACTTGCCGGGCAAGTCAGAGCATTGATAGGTATTTAC
ATTGGTACCAGCAGAAACCAGGGAAAGCCCCTAAGCTCCTGATCTATAGT
GCATCCGTGTTGCAAAGTGGGGTCCCATCACGTTTCAGTGGCAGTGGATC
TGGGACAGATTTCACCCTCACCATCAGCAGTCTGCAGCCTGAAGATTTTG
CTACGTACTACTGTCAACAGGTTGTGTGGCGTCCTTTTACGTTCGGCCAA
GGGACCAAGGTGGAAATCAAACGG
TAR1-5-27

(SEQ ID NO: 123)

GACATCCAGATGACCCAGTCTCCATCCTCCCTGTCTGCATCTGTAGGAGA
CCGTGTCACCATCACTTGCCGGGCAAGTCAGAGCATTGATAAGTATTTAC
ATTGGTACCAGCAGAAACCAGGGAAAGCCCCTAAGCTCCTGATCTATAGT
GCATCCTCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-28

(SEQ ID NO: 124)

GACATCCAGATGACCCAGTCTCCATCCTCCCTGTCTGCATCTGTAGGAGA
CCGTGTCACCATCACTTGCCGGGCAAGTCAGAGCATTGATCATTATTTAC
ATTGGTACCAGCAGAAACCAGGGAAAGCCCCTAAGCTCCTGATCTATAGT
GCATCCGTTTTGCAAAGTGGGGTCCCATCACGTTTCAGTGGCAGTGGATC
TGGGACAGATTTCACTCTCACCATCAGCAGTCTGCAACCTGAAGATTTTG
CAACGTAGTACTGTCAACAGGTTGTGTGGCGTCCTTTTACGTTCGGCCAA
GGGACCAAGGTGGAAATCAAACGG
TAR1-5-29

(SEQ ID NO: 125)

GACATCCAGATGACCCAGTCTCCATCCTCCCTGTCTGCATCTGTAGGAGA
CCGTGTCACCATCACTTGCCGGGCAAGTCAGAGCATTGATGAGTTTTTAC
ATTGGTACCAGCAGAAACCAGGGAAAGCCCCTAAGCTCCTGATCTATAGT
GCATCCATTTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-34

(SEQ ID NO: 126)

GACATCCAGATGACCCAGTCTCCATCCTCCCTGTCTGCATCTGTAGGAGA
CCGTGTCACCATCACTTGCCGGGCAAGTCAGAGCATTCAGACTGCGTTAC
TGTGGTACCAGCAGAAACCAGGGAAAGCCCCTAAGCTCCTGATCTATAAT
GCATCCAGTTTGCAAAGTGGGGTCCCATCACGTTTCAGTGGCAGTGGATC
TGGGACAGATTTCACTCTCACCATCAGCAGTCTGCAACCTGAAGATTTTG
CTACATACTACTGTCAACAGGTTGTGTGGCGTCCTTTTACGTTCGGCCAA
GGGACCAAGGTGGAAATCAAACGG
TAR1-5-35

(SEQ ID NO: 127)

GACATCCAGATGACCCAGTCTCCATCCTCCCTGTCTGCATCTGTAGGAGA
CCGTGTCACCATCACTTGCCGGGCAAGTCAGAGCATTGATCAGTATTTAC
ATTGGTACCAGCAGAAACCAGGGAAAGCCCCTAAGCTCCTGATCTATGGT
GCATCCAATTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-36

(SEQ ID NO: 128)

GACATCCAGATGACCCAGTCTCCATCCTCCCTGTCTGCATCTGTAGGAGA
CCGTGTCACCATCACTTGCCGGGCAAGTCAGAGGATTGATAATTATTTAC
ATTGGTACCAGCAGAAACCAGGGAAAGCCCCTAAGCTCCTGATCTATAGT
GCATCCCAGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-464

(SEQ ID NO: 129)

GACATCCAGATGACCCAGTCTCCATCCTCCCTGTCTGCATCTGTAGGAGA
CCGTGTCACCATCACTTGCCGGGCAAGTCAGAGCATTGATAATTTTTTAC
ATTGGTACCAGCAGAAACCAGGGAAAGCCCCTAAGCTCCTGATCTATAGT
GCATCCGAGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-463

(SEQ ID NO: 130)

GACATCCAGATGACCCAGTCTCCATCCTCCCTGTCTGCATCTGTAGGAGA
CCGTGTCACCATCACTTGCCGGGCAAGTCAGAGCATTGATGAGTATTTAC
ATTGGTACCAGCAGAAACCAGGGAAACCCCCTAAGCTCCTGATCTATTCT
GCATCCAGTTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-460

(SEQ ID NO: 131)

GACATCCAGATGACCCAGTCTCCATCCTCCCTGTCTGCATCTGTAGGAGA
CCGTGTCACCATCACTTGCCGGGCAAGTCAGAGCATTGATCATTTTTTAC
ATTGGTACCAGCAGAAACCAGGGAAAGCCCCTAAGCTCCTGATCTATAGT
GCATCCGAGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-461

(SEQ ID NO: 132)

GACATCCAGATGACCCAGTCTCCATCCTCCCTGTCTGCATCTGTAGGAGA
CCGTGTCACCATCACTTGCCGGGCAAGTCAGAGCATTGATAATTATTTAC
ATTGGTACCAGCAGAAACCAGGGAAAGCCCCTAAGCTCCTGATCTATTCG
GCATCCATGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-479

(SEQ ID NO: 133)

GACATCCAGATGACCCAGTCTCCATCCTCCCTGTCTGCATCTGTAGGAGA
CCGTGTCACCATCACTTGCCGGGCAAGTCAGAGCATTGATGAGTATTTAC
ATTGGTACCAGCAGAAACCAGGGAAAGCCCCCAAGCTCGTGATCTATTCT
GCATCCATTTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-477

(SEQ ID NO: 134)

GACATCCAGATGACCCAGTCTCCATCCTCCCTGTCTGCATCTGTAGGAGA
CCGTGTCACCATCACTTGCCGGGCAAGTCAGAGCATTGATGAGTTTTTAC
ATTGGTACCAGCAGAAACCAGGGAAAGCCCCTAAGCTCCTGATCTATTCG
GCATCCGCTTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1-5-478

(SEQ ID NO: 135)

GACATCCAGATGACCCAGTCTCCATCCTCCCTGTCTGCATCTGTAGGAGA
CCGTGTCACCATCACTTGCCGGGCAAGTCAGAGCATTGATGAGTATTTAC
ATTGGTACCAGCAGAAACCAGGGAAAGCCCCTAAGCTCCTGATCTATTCT
GCATCCATTTTGCAAAGTGGGGTCCCATCACGTTTCAGTGGCAGTGGATC
TGGGACAGATTTCACTCTCACCATCAGCAGTCTGCACCCTGAAGATTTTG
CTACGTACTACTGTCAACAGGTTGTGTGGCGTCCTTTTACGTTCGGCCAA
GGGACCAAGGTGGAAATCAAACGG
TAR1-5-476

(SEQ ID NO: 136)

GACATCCAGATGACCCAGTCTCCATCCTCCCTGTCTGCATCTGTAGGAGA
CCGTGTCACCATCACTTGCCGGGCAAGTCAGAGCATTGATAATTATTTAC
ATTGGTACCAGCAGAAACCAGGGAAAGCCCCTAAGCTCCTGATCTATGCT
GCATCCAGTTTGCAAAGTGGGGTCCCATCACGTTTCAGTGGCAGTGGATC
TGGGACAGATTTCACTCTCACCATCAGCAGTCTGCAACCTGATGATTTTG
CTACGTACTACTGTCAACAGGTTGTGTGGCGTCCTTTTGCGTTCGGCCAA
GGGACCAAGGTGGAAATCAAACGG
TAR1-5-490

(SEQ ID NO: 137)

GACATCCAGATGACCCAGTCTCCATCCTCCCTGTCTGCATCTGTAGGAGA
CCGTGTCACCATCACTTGCCGGGCAAGTCAGAGCATTGATAGTTATTTAC
ATTGGTACCAGCAGAAACCAGGGAAAGCCCCTAAGCTCCTGATCTATAGT
GCATCAAATTTAGAAACAGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGGTTGTGTGGCGTCCTTTTACGTTCGGCCAA
GGGACCAAGGTGGAAATCAAACGG
TAR1h-1

(SEQ ID NO: 138)

GACATCCAGATGACCCAGTCTCCATCCTCCCTGTCTGCATCTGTAGGAGA
CCGTGTCACCATCACTTGCCGGGCAAGTCAGGTGATTTGGGATGCGTTAG
ATTGGTACCAGCAGAAACCAGGGAAAGCCCCTAAGCTCCTGATCTATAGT
GCGTCCCGTTTGCAAAGTGGGGTCCCATCACGTTTCAGTGGCAGTGGATC
TGGGACAGATTTCACTCTCACCATCAGCAGTCTGCATCCTGAAGATTTTG
CTACGTACTACTGTCAACAGTATGCTGTGTTTCCTGTGACGTTCGGCCAA
GGGACCAAGGTGGAAATCAAACGG
TAR1h-2

(SEQ ID NO: 139)

GACATCCAGATGACCCAGTCTCCATCCTCCCTGTCTGCATCTGTAGGAGA
CCGTGTCACCATCACTTGCCGGGCAAGTCAGACTATTTATGATGCGTTAA
GTTGGTACCAGCAGAAACCAGGGAAAGCCCCTAAGCTCCTGATCTATGGT
GGTTCCAGGTTGCAAAGTGGGGTCCCATCACGTTTCAGTGGCGGTGGATC
TGGGACAGATTTCACTCTCACCATCAGCAGTCTGCAACCTGAAGATTTTG
CTACGTACTACTGTCAACAGTATAAGACTAAGCCTTTGACGTTCGGCCAA
GGGACCAAGGTGGAAATCAAACGG
TAR1h-3

(SEQ ID NO: 140)

GACATCCAGATGACCCAGTCCCCATCCTCCCTGTCTGCATCTGTAGGAGA
CCGTGTCACCATCACTTGCCGGGCAAGTCAGACTATTTATGATGCGTTAA
GTTGGTACCAGCAGAAACCAGGGAAAGCCCCTAAGCTCCTGATCTATGGT
GGTTCCAGGTTGCAAAGTGGGGTCCCATCACGTTTCAGTGGTAGTGGATC
TGGGACAGATTTCACTCTCACCATCAGCAGTCTGCAACCCGAAGATTTTG
CTACGTACTACTGTCAACAGTATGCTCGTTATCCTCTTACGTTCGGCCAA
GGGACCAAGGTGGAAATCAAACGG

Additional anti-human TNF-α dAb clones include the following:

Several clones have been subjected to affinity maturation. Clone TAR1-100-47 is an affinity-matured clone with an ND50 of 30-50 nM in the L929 cell assay, and 3-5 nM when cross-linked with protein L. TAR1-100-47 cross-reacts with rhesus TNF. Its amino acid sequence and those of a number of other clones are as provided bleow. TAR1-2-100 and TAR1-2-109 are parent clones used for construction of the library. The good TAR1 clones in this group have the following consensus sequence:

D/E30, W32, R94 and F96, as indicated in bold in
TAR1-100-47
TAR1-100-29,

(SEQ ID NO: 141)

DIQMTQSPSSLSASVGDRVTITCRASQDIEEWLMWYQQKPGKAPKLLIYN
SSTLQSGVPSRFSGSGSGTDFTLTISSLQPEDYATYYCQQPLSRPFTFGQ
GTKVEIKR
TAR1-100-35

(SEQ ID NO: 142)

DIQMTQSPSSLSASVGDRVTITCRASQHIDDWLFWYQQKPGKAPKLLIYR
ASFLQSGVPSRFSGSGSGTDFTLTISSLQPEDFATYYCQQPLSRPFTFGQ
GTKVEIKR
TAR1-100-43

(SEQ ID NO: 143)

DIQMTQSPSSLSASVGDRVTITCRASQFIEDWLFWYQQKPGKAPKLLIYQ
ASKLQSGVPSRFSGSGSGTDFTLTISSLQPEDFATYYCQQPLSRPFTFGQ
GTKVEIKR
TAR1-100-47

(SEQ ID NO: 144)

DIQMTQSPSSLSASVGDRVTITCRASQPIDSWLMWYQQKPGKAPKLLIYQ
ASRLQSGVPSRFSGSGSGTDFTLTISSLQPEDFATYYCQQPLSRPFTFGQ
GTKVEIKR
TAR1-100-52

(SEQ ID NO: 145)

DIQMTQSPSSLSASVGDRVTITCRASQHIDDWLFWYQQKPGKAPKLLIYR
ASFLQSGVPPRFSGSGSGTDFTLTISSLQPEDFATYYCQQPLSRPFTFGQ
GTKVEIKR
TAR1-109

(SEQ ID NO: 146)

DIQMTQSPSSLSASVGDRVTITCRASQNIDDHLMWYQQKPGKAPKLLIYS
SSILQSGVPPRFSGSGSGTDFTLTISSLQPEDFATYYCQQPLSRPFTFGQ
GTKVEIKR
TAR1-100

(SEQ ID NO: 147)

DIQMTQSPSSLSASVGDRVTITCRASQDIDHALLWYQQKPGKAPRLLIYN
GSMLQSGVPSRFSGSGSGTDFTLTISSLQPEDFATYYCQQVLRRPFTFGQ
GTKVEIKR
TAR1-100-34

(SEQ ID NO: 148)

DIQMTQSPSSLSASVGDRVTITCRASQHIGDWLLWYQQKPGKAPMLLIYQ
SSRLQSGVPSRFSGSGSGTDFILTISSLQPEDFATYYCQQPLSRPFTFGQ
GTKVEIKR
TAR1-100-36

(SEQ ID NO: 149)

DIQMTQSPSSLSASVGDRVTITCRASQHIDSYLMWYQQKPGKAPKLLIYN
TSVLQSGVPSRFSGSGSGTDFTLTISSLQPEDFATYYCQQPLSRPFTFGQ
GTKVEIKR
TAR1-100-38

(SEQ ID NO: 150)

DIQMTQSPSSLSASVGDRVTITCRASQWIDDHLFWYQQKPGKAPKLLIYN
TSTLQSGVPSRFSGSGSGTDFILTISSLQPEDFATYYCQQPLSRPFTFGQ
GTKVEIKR
TAR1-100-39

(SEQ ID NO: 151)

DIQMTQSPSSLSASVGDRVTITCRASQFIDEHLMWYQQKPGKAPKLLIYR
SSELQSGVPSRFSGSGSGTDFTLTISSLQPEDFATYYCQQPLSRPFTFGQ
GTKVEIKR
TAR1-100-40

(SEQ ID NO: 152)

DIQMTQSPSSLSASVGDRVTITCRASQWINNWLLWYQQKPGKAPKLLIYE
SSNLQSGVPSRFSGSGSGTDFTLTISSLQPEDFATYYCQQPLSRPFTFGQ
GTKVEIKR
TAR1-100-41

(SEQ ID NO: 153)

DIQMTQSPSSLSASVGDRVTITCRASQLIDDHFWYQQKPGKAPTLLIYNS
SVLQSGVPSRFSGSGSGTDFTLTISSLQPEDFATYYCQQPLSRPFTFGQG
TKVEIKR
TAR1-100-45

(SEQ ID NO: 154)

DIQMTQSPSSLSASVGDRVTITCRASQDIDQWLMWYQQKPGKAPKLLIYQ
SSMLQSGVPSRFSGSGSGTDFTLTISSLQPEDFATYYCQQPLSRPFTFGQ
GTKVEIKR
TAR1-100-60

(SEQ ID NO: 155)

DIQMTQSPSSLSASVGDRVTITCQASQDIDNWLLWYQQKPGKAPKLLIYQ
ASNLQSGVPSRFSGSGSGTDFTLTISSLQPEDFATYYCQQPLSRPFTFGQ
GTKVEIKR
TAR1-100-62

(SEQ ID NO: 156)

DIQMTQSPSSLSASVGDRVTITCRASQPIDSWLMWYQQKPGKAPKLLIYQ
ASRLQSGVPSRFSGSGSGTDFTLTISSLQPEDFATYYCQQPLSGPFTFGQ
GTKVEIKR
TAR1-100-64

(SEQ ID NO: 157)

DIQMTQSPSSLSASVGDRVTITCRASQYIDYGLMWYQQKPGKAPKLLIYR
TSELQSGVPSRFSGSGSGTDFTLTISSLQPEDFATYYCQQPLSRPFTFGQ
GTKVEIKR
TAR1-100-65

(SEQ ID NO: 158)

DIQMTQSPSSLSASVGDRVTITCRASQWIDSFLMWYQQKPGKAPKLLIYN
GSVLQSGVPSRFSGSGSGTDFTLTISSLQPEDFATYYCQQPLSRPFTFGQ
GTKVEIKR
TAR1-100-75

(SEQ ID NO: 159)

DIQMTQSPSSLSASVGDRVTITCRASQDIGPWLMWYQQKPGKAPKLLIYQ
GSRLQSGVPLRFSGSGSGTDFTLTISSLQPEDFATYYCQQPLSRPFTFGQ
GTKVEIRR
TAR1-100-76

(SEQ ID NO: 160)

DIQMTQSPSSLSASVGDRVTITCRASQHIDSWLLWYQQKPGKAPKLLIYN
GSVLQSGVPSRFSGSGSGTDFTLTISSLQPEDFATYYCQQPLSGPFTFGQ
GTKVEIKR
TAR1-100-77

(SEQ ID NO: 161)

DIQMTQSPSSLSASVGDRVTITCRASQHIDTHLFWYQQKPGKAPKLLIYN
TSTLQSGVPSRFSGSGSGTDFTLTISSLQPEDFATYYCQQPLSRPFTFGQ
GTKVEIKR
TAR1-100-78

(SEQ ID NO: 162)

DIQMTQSPSSLSASVGDRVTITCRASQFIDTHLMWYQQKPGKAPRLLIYN
TSTLQSGVPSRFSGSGSGTDFTLTISSLQPEDFATYYCQQPLSRPFTFGQ
GTKVEIKR
TAR1-100-80

(SEQ ID NO: 163)

DIQMTQSPSSLSASVGDRVTITCRASQDIDDWLLWYQQKPGKAPKLLIYQ
GSRLQSGVPSRFSGSGSGTDFTLTISSLQPEDFATYYCQQPLSRPFTFGQ
GTKVEIKR
TAR1-100-82

(SEQ ID NO: 164)

DIQMTQSPSSLSASVGDRVTITCRASQWIDDTLMWYQQKPGKAPKLLIYR
SSMLQSGVPSRFSGSGSGTDFTLTISSLQPEDFATYYCQQPLSRPFTFGQ
GTKVEIKR
TAR1-100-83

(SEQ ID NO: 165)

DIQMTQSPSSLSASVGDRVTITCRASQYIDSHLMWYQQKPGKAPKLLIYD
TSRLQSGVPSRFSGSGSGTDFTLTISSLQPEDFATYYCQQPLSRPFTFGQ
GTKVEIKR
TAR1-100-84

(SEQ ID NO: 166)

DIQMTQSPSSLSASVGDRVTITCRASQHIDQHLFWYQQKPGKAPKLLIYN
SSSLQSGVPSRFSGSGSGTDFTLTISSLQPEDFATYYCQQPLSRPFTFGQ
GTKVEIKR
TAR1-100-89

(SEQ ID NO: 167)

DIQMTQSPSSLSASVGDRVTITCRASQHIERWLLWYQQKPGKAPKLLIYN
SSKLQSGVPSRFSGSGSGTDFTLTISSLQPEDFATYYCQQPLSRPFTFGQ
GTKVEIKR
TAR1-100-90

(SEQ ID NO: 168)

DIQMTQSPSSLSASVGDRVTISCRASQHIERWLLWYQQKPGKAPKLLIYN
SSKLQSGVPSRFSGSGSGTDFTLTISSLQPEDFATYYCQQPLSRPFTFGQ
GTKVEIKR
TAR1-100-91

(SEQ ID NO: 169)

DIQMTQSPSSLSASVGDRVTITCRASQDIGSWLMWYQQKSGKAPKLLIYN
GSALQSGVPSRFSGSGSGTDFTLTISSLQPEDFATYYCQQPLSRPFTFGQ
GTKVEIKR
TAR1-100-92

(SEQ ID NO: 170)

DIQMTQSPSSLSASVGDRVTITCRASQHIDKWLMWYQQKPGKAPKLLIYQ
ASKLQSGVPSRFSGSGSGTDFTLTISSLQPEDFATYYCQQPLSRPFTFGQ
GTKVEIKR
TAR1-100-93

(SEQ ID NO: 171)

DIQMTQSPSSLSASVGDRVTITCRASQDIEEWLMWYQQKPGKAPKLLIYN
SSTLQSGVPSRFSGSGSGTDFTLTISSLQPEDFATYYCQQPLSRPFTFGQ
GTKVEIKR
TAR1-100-94

(SEQ ID NO: 172)

DIQMTQSPSSLSASVGDRVTITCRASQYIDYGLMWYQQKPGKAPKLLIYR
TSELQSGVPSRFSGSGSGTDFTLTISSLQPEDFATYYCQQPLSRPFTFGQ
TAR1-100-95

(SEQ ID NO: 173)

DIQMTQSPSSLSASVGDRVTITCRASQNIDIHLMWYQQKPGKAPKLLIYQ
SSNLQSGVPSPFSGSGSGTDFTLTISSLQPEDFATYYCQQPLSRPFTFGQ
GTKVEIKR
TAR1-100-96

(SEQ ID NO: 174)

DIQMTQSPSSLSASVGDRVTITCRASQDIGPWLLWYQQKIPGKAPKLLIY
QSSELQSGVPSRFSGSGSGTDFTLTISSLQPEDLATYYCQQPLSRPFTFG
QGTKVEIKR
TAR1-100-97

(SEQ ID NO: 175)

DIQMTQSPSSLSASVGDRVTITCRASQEIGVWLMWYQQKPGKAPKLLIYE
GSRLQSGVPSRFSGSGSGTDFTLTISSLQPEDFATYYCQQPLSRPFVFGQ
GTKVEIKR
TAR1-100-98

(SEQ ID NO: 176)

DIQMTQSPSSLSASVGDRVTITCRASQSIGKWLMWYQQKPGKAPKLLIYQ
SSLLQSGVPSRFSGSGSGTDFTLTISSLQPEDFATYYCQQPLSRPFTFGQ
GTKVEIKR
TAR1-100-99

(SEQ ID NO: 177)

DIQMTQSPSSLSASVGDRVTITCRASQDIDTWLFWYQQKPGKAPKLLIYN
GSRLQSGVPSRFSGSGSGTDFTLTISGLQPEDFATYYCQQPLSRPFTFGQ
GTKVEIKR
TAR1-100-100

(SEQ ID NO: 178)

DIQMTQSPSSLSASVGDRVTITCRASQPIDSWLMWYQQKPGKAPKLLIYQ
ASRLQSGVPSRFSGSGSGTDFTLTISSLQPEDFATYYCQQPLSRPFTFGQ
GTKVEIKR
TAR1-100-101

(SEQ ID NO: 179)

DIQMTQSPSSLSASVGDRVTITCRASQDIEGWLLWYQQKPGKAPKLLIYN
SSTLQSGVPSRFSGSGSGTDFTLTISSLQPEDFATYYCQQPLSRPFTFGQ
GTKVEIKR
TAR1-100-102

(SEQ ID NO: 180)

DIQMTQSPSSLSASVGDRVTITCRASQHIDDWLFWYQQKPGKAPKLLIYR
ASFLQSGVPSRFSGSGSGTDFTLTISSLQPEDFATYYCQQPLSRPFTFGQ
GTKVEIKR
TAR1-100-103

(SEQ ID NO: 181)

DIQMTQSPSSLSASVGDRVTITCRASQDIDTWLFWYQQKPGKAPKLLIYN
GSRLQSGVPSRFSGSGSGTDFTLTISGLQPEDFATYYCQQPLSRPFTFGQ
GTKVEIKR
TAR1-100-105

(SEQ ID NO: 182)

DIQMTQSPSSLSASVGDRVTITCRASQPIEEWLLWYQQKPGKAPKLLIYN
GSHLQSGVPSRFSGSGSGTDFTLTISSLQPEDFATYYCQQPLSRPFTFGQ
GTKVEIKR
TAR1-100-106

(SEQ ID NO: 183)

DIQMTQSPSSLSASVGDRVTITCRASQHIDKWLMWYQQKPGKAPKLLIYQ
ASKLQSGVPSRFSGSGSGTDFTLTISSLQPEDFATYYCQQPLSRPFTFGQ
GTKVEIKR
TAR1-100-107

(SEQ ID NO: 184)

DIQMTQSPSSLSASVGDRVTITCRASQDIEEWLMWYQQKPGKAPKLLIYN
SSTLQSGVPSRFSGSGSGTDFTLTISSLQPEDYATYYCQQPLSRPFTFGQ
GTKVEIKR
TAR1-100-108

(SEQ ID NO: 185)

DIQMTQSPSSLSASVGDRVTITCRASQPIDYGLMWYQQKPGKAPKLLIYR
SSQLQSGVPSRFSGSGSGTDFTLTISSLQPEDFATYYCQQPLSRPFTFGQ
GTKVEIKR
TAR1-100-109

(SEQ ID NO: 186)

DIQMTQSPSSLSASVGDRVTITCRASQEIGSWLMWYQQKPGKAPKLLIYQ
SSKLQSGVPSRFSGSGSGTDFTLTISSLQPEDFATYYCQQPLSRPFTFGQ
GTKVEIKR
TAR1-100-110

(SEQ ID NO: 187)

DIQMTQSPSSLSASVGDRVTITCRASQPIDSWLLWYQQKPGKAPKLLIYN
ASSLQSGVPSRESGSGSGTDFTLTISSLQPEDFATYYCQQPLSRPFTFGQ
GTKVEIKR
TAR1-100-111

(SEQ ID NO: 188)

DIQMTQSPSSLSASVGDRVTITCRASQDIGPWLMWYQQKPGKAPKLLIYQ
ASALQSGVPSRFSGSGSGTDFTLTISSLQPEDFATYYCQQPLSRPFTFGQ
GTKVEIKR
TAR1-100-112

(SEQ ID NO: 189)

DIQMTQSPSSLSASVGDRVTITCRASQNIHEWLMWYQQKPGKAPKLLIYQ
GSRLQSGVPSRFSGSGSGTDFTLTISSLQPEDFATYYCQQPLSRPFTFGQ
GTKVEIKR
TAR1-100-113

(SEQ ID NO: 190)

DIQMTQSPSSLSASVGDRVTITCRASQDIGPWLMWYQQKPGKAPKLLIYQ
ASALQSGVPSRFSGSGSGTDFTLTISSLQPEDSATYYCQQPLSRLPFTFG
QGTKVEIKR

The sequence of the TAR1-5-19 anti-human TNF-α dAb adapted to various formats in these examples is as follows:

TAR1-5-19
Amino acid

(SEQ ID NO: 191)

DIQMTQSPSSLSASVGDRVTITCRASQSVKEFLWWYQQKPGKAPKLLIYM
ASNLQSGVPSRFSGSGSGTDFTLTISSLQPEDFATYYCQQKFKLPRTFGQ
GTKVEIKR
Nucleotide

(SEQ ID NO: 115)

gacatccagatgacccagtctccatcctccctgtctgcatctgtaggaga
ccgtgtcaccatcacttgccgggcaagtcagagcgttaaggagtttttat
ggtggtaccagcagaaaccagggaaagcccctaagctcctgatctatatg
gcatccaatttgcaaagtggggtcccatcacgtttcagtggcagtggatc
tgggacagatttcactctcaccatcagcagtctgcaacctgaagattttg
ctacgtactactgtcaacagaagtttaagctgcctcgtacgttcggccaa
gggaccaaggtggaaatcaaacgg

### Example 21

### Efficacy Study of PEGylated TAR1-5-19 in a Prophylactic Model of Arthritis

Tg197 mice are transgenic for the human TNF-globin hybrid gene and heterozygotes at 4-7 weeks of age develop a chronic, progressive polyarthritis with histological features in common with rheumatoid arthritis [Keffer , J., Probert, L., Cazlaris, H., Georgopoulos, S.,Kaslaris, E., Kioussis, D., Kollias, G. (1991). Transgenic mice expressing human tumor necrosis factor: a predictive genetic model of arthritis. EMBO J., Vol. 10, pp. 4025-4031.]

To test the efficacy of a PEGylated dAb (PEG format being 2×20 k branched with 2 sites for attachment of the dAb [i.e. 40K mPEG2 MAL2], the dAb being TAR1-5-19cys) in the prevention of arthritis in the Tg197 model, heterozygous transgenic mice were divided into groups of 10 animals with equal numbers of male and females. Treatment commenced at 3 weeks of age with weekly intraperitoneal injections of test items. The expression and PEGylation of TAR1-5-19cys monomer is outlined in Section 1.3.3, example 1. All protein preparations were in phosphate buffered saline and were tested for acceptable levels of endotoxins.

The study was performed blind. Each week the animals were weighed and the macrophenotypic signs of arthritis scored according to the following system: 0=no arthritis (normal appearance and flexion), 1=mild arthritis (joint distortion), 2=moderate arthritis (swelling, joint deformation), 3=heavy arthritis (severely impaired movement).

The outcome of the study clearly demonstrated that 10 mg/kg PEGylated TAR1-5-19 inhibited the development of arthritis with a significant difference between the arthritic scoring of the saline control and treated group. The 1 mg/kg dose of PEGylated TAR1-5-19 also produced a statistically significantly lower median arthritic score than saline control group (P<0.05% using normal approximation to the Wilcoxon Test).

### Example 22

### Efficacy Study of PEGylated TAR1-5-19 in a Therapeutic Model of Arthritis

To test the efficacy of a PEGylated dAb in the therapeutic model of arthritis in the Tg197 model, heterozygous transgenic mice were divided into groups of 10 animals with equal numbers of male and females. Treatment commenced at 6 weeks of age when the animals had significant arthritic phenotypes. Treatment was twice weekly with 4.6 mg/kg intraperitoneal injections of test items. The sample preparation and disease scoring are as described above in Example 21.

The arthritic scoring clearly demonstrated that PEGylated TAR1-5-19 inhibited the progression of arthritis in a therapeutic model. The 4.6 mg/kg dose of PEGylated TAR1-5-19 produced a statistically significantly lower median arthritic score than saline control group at week 9 (P<0.01% using normal approximation to the Wilcoxon Test).

### Example 23

### dAb Efficacy in a Slow Release Format

To test the efficacy of a dAb from a slow release format, a dAb with a small PEG molecule (where the PEG is 4×5 k with four sites for attachment of a dAb with a C-terminal cys residue, the dAb being TAR1-5-19 [i.e. 20K PEG 4 arm MAL]) was loaded into a 0.2 ml osmotic pump. The pump had a release rate of 0.2 ml over a 4 week period was implanted subcutaneously into mice at week 6 in the therapeutic Tg197 model as described above. The arthritic scores of these animals increased at a clearly slower rate when compared to animals implanted with pumps loaded with saline. This demonstrates that dAbs are efficacious when delivered from a slow release format.

### Example 24

### Half-Life Stabilized Anti-Human TNF-α dAb Prevents the Onset of RA in the Tg197 Mouse Model

The dAb monomer TAR1-5-19 described herein is an affinity matured dAb monomer derived from a dAb initially selected using passively coated TNF-α. The initial clone had a ND50 in the L929 TNF-cytotoxicity neutralization assay greater than 5 μM. TAR1-5-19 has an ND50 of less than 30 nM. When formatted as an Fc Fusion as described herein, the TAR1-5-19 clone has an ND50 of less than 5 nM in the L929 assay.

The serum half-life of TAR1-5-19 dAb Fc-fusion was examined following injection into mice. Results are shown in FIG. 35. Where the TAR1-5-19 dAb monomer had a t½β of approximately 20 minutes, the Fc-fusion formatted version of the same dAb had a t½β of greater than 24 hours, representing a greater than 70-fold increase in serum half-life.

The TAR1-5-19 dAb Fc fusion construct was tested in the Tg197 mouse model of RA described herein above. Mice were divided into five groups of 10, with equal numbers of male and female mice per group. Treatment with twice weekly IP injections of TAR1-5-19 dAb Fc fusion, ENBREL or saline was begun at 3 weeks of age, a time at which RA symptoms have not yet manifested. The study was conducted for 7 weeks. As shown in FIG. 36, two dosages of the TAR1-5-19 dAb Fc fusion, 1 mg/kg and 10 mg/kg, were administered. Negative control animals received a negative control anti-β-gal Fc fusion twice weekly at 10 mg/kg, and one group was treated twice weekly with saline injection. For comparison, one group received 10 mg/kg of ENBREL twice weekly.

Animals were assessed for arthritic scores as described herein, in a blinded manner. At the end of the 7 week course of treatment, animals receiving the twice weekly dosage of 10 mg/kg of the TAR1-5-19 dAb Fc fusion had lower arthritic scores than the animals receiving ENBREL at 10 mg/kg, and had experienced essentially complete prevention of arthritic disease relative to non-treated animals or animals receiving the negative control dAb Fc fusion.

TNF-α is associated with cachexia. Animals were weighed throughout the course of anti-TNF-α dAb treatment. The weights of the animals receiving the TAR1-5-19 dAb Fc fusion were significantly greater than those receiving negative control dAb Fc fusion and no treatment and similar to the weights of the animals receiving ENBREL injections.

In summary, 10 mg/kg TAR1-5-19 completely prevented the onset of arthritis in the Tg197 model. This response was dose-dependent, with a partial effect resulting from a 1 mg/kg dose, and the response was superior to that observed with a similar dose of the existing anti-TNF-α drug ENBREL. This study demonstrates the efficacy of dAbs as therapeutics in a clinically accepted model of human disease.

Histopathological analyses of fixed sections from the joints of the animals are in agreement with these data (not shown).

### Example 25

### In Vivo Studies on Differing Extended Half-Life Formats

In one series of studies, three different extended half-life anti-TNF-α dAb formats were examined for their effect on arthritic score. These formats were an anti-TNF-α dAb Fc fusion (two anti-human TNF-α dAbs homodimerized by fusion to human IgG C_{H}2/C_{H}3 region), two different PEG-linked anti-TNF-α dAb constructs (a homodimer formed by the cys-maleimide linkage of two identical dAbs to a 2×20K branched PEG and a homotetramer formed by the cys-maleimide linkage of four identical dAbs to a 4×10K branched PEG) and a dual-specific anti-TNF-α/Anti SA dAb comprising two identical anti-TNF-α dAbs followed by an anti-mouse serum albumin dAb.

In separate studies, drug compositions were administered either weekly at 10 mg/kg or 1 mg/kg as shown in FIG. 37 or twice weekly at varying doses, commencing at 3 weeks of age and continuing for 7 weeks.

The PEGylated anti-TNF dAb homodimer was effective at 10 mg/kg in the weekly injection protocol for the complete prevention of arthritis based on arthritic score. Current anti-TNF-α drug used for comparison had a reduced arthritic score relative to untreated animals, but the score was higher in a statistically significant manner than the score achieved with the PEGylated dAb construct. The anti-TNF-α/anti-SA dual specific and the Fc fusion showed effect relative to no treatment.

In the 1 mg/kg weekly injection regimen, while none of the treatments was 100% effective at preventing the onset of disease, the PEGylated anti-TNF-α dAb construct was still highly effective in preventing the progression of disease symptoms relative to no treatment and current anti-TNF-α drug. In this dosing regimen, the anti-TNF-α dAb Fc fusion and the dual-specific construct were also more effective than the current drug.

In summary, the weekly dosing regimen studies with three different formats of half-life-extended dAbs further validates the efficacy of treatment in a clinically accepted model of human disease.

### Example 26

### Efficacy of Anti-Human TNF-α dAbs in the Tg197 Mouse RA Model Relative to Existing Anti-TNF-α Therapeutics Against Established Disease

In this study, the efficacy of various formats and dosage regimens of anti-TNF-α dAb constructs against established disease was compared to that of equal molar doses of the current anti-TNF-α therapeutics ENBREL, HUMIRA and REMICADE in the Tg197 RA model. Animals were administered the therapeutics starting at 6 weeks, instead of at 3 weeks, such that arthritic symptoms had already manifested. Symptoms were monitored by histology (at 9 weeks) and arthritic scoring (weekly) in a blinded manner.

The various formats and dosages for twice-weekly administration are shown in FIG. 38. Formats included the Fc fusion (two copies of the TAR1-5-19 dAb homodimerized by fusion to human IgG1 C_{H}2/C_{H}3 region), the TAR1-5-19 dAb PEG dimer (a homodimer formed by the cys-maleimide linkage of two identical dAbs to a 2×20K branched PEG), the TAR1-5-19 dAb PEG tetramer (a homotetramer formed by the cys-maleimide linkage of four identical dAbs to a 4×10K branched PEG), the TAR1-5-19 dAb/anti mouse SA dual-specific (linear fusion of two identical anti-TNF-α dAbs followed by an anti-mouse serum albumin dAb). The dosing regimen is shown schematically in FIG. 39. Continuous administration of a 4×5 k PEGylated TAR1-5-19 construct via an implanted osmotic pump was also evaluated.

The results of the study showed not one of the current biologics appreciably reversed the arthritic score by 9 weeks. The TAR formats all to a greater or lesser degree stabilized the arthritic score when compared with the saline control, and this was statistically significant. Moreover when compared with the week 6 score there were signs of disease reversal.

The arthritic joints at week 9 when examined for histopathological disease status also showed a reduction in disease severity following treatment with the TAR formats when compared with the joints at week 6. This confirms that the TAR formats can elicit a reversal of the arthritic phenotype of the established disease.

These studies demonstrate the effectiveness of the tested anti-TNF-α dAb constructs against established arthritic disease, including the ability of a TNF-α dAb to at least partially reverse the course of disease.

### Example 27

### Efficacy of an Anti-TNF dAb as a Fusion with an Anti-Serum Albumin dAb

A Efficacy study of TAR1-5-19/anti-serum albumin dAb fusion a prophylactic model of arthritis.

Tg197 mice are transgenic for the human TNF-globin hybrid gene and heterozygotes at 4-7 weeks of age develop a chronic, progressive polyarthritis with histological features in common with rheumatoid arthritis [Keffer, J., Probed, L., Cazlaris, H., Georgopoulos, S., Kaslaris, E., Kioussis, D., Kollias, G. (1991). Transgenic mice expressing human tumour necrosis factor: a predictive genetic model of arthritis. EMBO J., Vol. 10, pp. 4025-4031.]

To test the efficacy of a TAR1-5-19/anti-serum albumin dAb fusion (a inline trimer of 3 dAbs, being TAR1-5-19, TAR1-5-19 and an anti-mouse serum albumin dAb) in the prevention of arthritis in the Tg197 model, heterozygous transgenic mice were divided into groups of 10 animals with equal numbers of male and females. Treatment commenced at 3 weeks of age with weekly intraperitoneal injections of test items. TAR1-5-19/anti-serum albumin dAb fusion was expressed in E. coli with a C-terminal hexa histidine tag and purified by Ni affinity chromatography, IEX and gel filtration. All protein preparations were in phosphate buffered saline and were tested for acceptable levels of endotoxins.

The study was performed blind. Each week the animals were weighed and the macrophenotypic signs of arthritis scored according to the following system: 0=no arthritis (normal appearance and flexion), 1=mild arthritis (joint distortion), 2=moderate arthritis (swelling, joint deformation), 3=heavy arthritis (severely impaired movement).

The outcome of the study clearly demonstrated that 10 mg/kg TAR1-5-19/anti-serum albumin dAb fusion inhibited the development of arthritis with a significant difference between the arthritic scoring of the saline control and treated group. The 1 mg/kg dose of TAR1-5-19/anti-serum albumin dAb fusion also produced a statistically significantly lower median arthritic score than saline control group (P<2% using normal approximation to the Wilcoxon Test).

B Efficacy Study of TAR1-5-19/Anti-Serum Albumin dAb Fusion in a Therapeutic Model of Arthritis

To test the efficacy of a TAR1-5-19/anti-serum albumin dAb fusion in the therapeutic model of arthritis in the Tg197 model, heterozygous transgenic mice were divided into groups of 10 animals with equal numbers of male and females. Treatment commenced at 6 weeks of age when the animals had significant arthritic phenotypes. Treatment was twice weekly with 2.7 mg/kg intraperitoneal injections of test items. The sample preparation and disease scoring are as described above.

The arthritic scoring clearly demonstrated that TAR1-5-19/anti-serum albumin dAb fusion inhibited the progression of arthritis in a therapeutic model. The 2.7 mg/kg dose of TAR1-5-19/anti-serum albumin dAb fusion produced a statistically significantly lower median arthritic score than saline control group at week 9 (P<0.05% using normal approximation to the Wilcoxon Test).

This clearly demonstrates that anti-TNF dAbs can be effective in a format with anti-SA dAbs and that the anti-SA dAb has extended the serum half life of the anti-TNF dAb from that which would be expected for an anti-TNF dAb alone.

### Example 28

### Examination of the Effects of Anti-TNF-α dAbs as Disclosed Herein on Arthritic and Histopathological Scores in the Tg197 Mouse Model of RA

Two additional studies were carried out examining the effects of anti-TNF-α dAbs on arthritic and histopathologic scores in the Tg197 model of RA.

In the first study, a TAR1-5-19 dAb Fc fusion as described above was administered at 10 mg/kg, twice weekly commencing at 3 weeks of age—before the onset of RA symptoms. Results were judged in comparison with saline, ENBREL and control Fc fusion dAb injection on the same schedule.

The TAR1-5-19 dAb Fc fusion was more effective than ENBREL in preventing the onset of RA symptoms in the mice as judged by arthritic score and from analysis of the histology slides.

In the second study, the effects of weekly injections of anti-TNF-α dAb Fc fusion, PEG dimer and dual-specific anti-TNF/antiSA at 10 or 1 mg/kg, commencing at 3 weeks of age. Comparison is to ENBREL and HUMIRA.

The arthritic scores for all the TAR formats, given as either 1 mg/kg or 10 mg/kg doses, were reduced when compared with the saline control. Moreover there was evidence of a delay in the onset of the disease. The PEGylated and anti-SA dual specific formats were more effective at reducing the severity of the arthritis when compared with Humira and Enbrel. In addition analysis of the histology of the joints at week 10 also showed that the TAR formats had been efficacious and reduced the disease severity when compared with the saline control.

In summary, the TAR1-5-19 anti-TNF-dAb in the Fc fusion, PEGylated and anti-SA dual specific formats are all effective against RA symptoms in the Tg197 model system, whether administered before or after the onset of arthritic symptoms. The most effective anti-TNF dAb formats are either equivalent to or more effective than HUMIRA, and the most effective anti-TNF dAb formats are significantly more effective than ENBREL in all studies.

### Example 29

### Anti-Human VEGF dAbs

TAR15 (Anti-Human VEGF)

VK dAbs that bind human VEGF are described below. RBA refers to the VEGF receptor 2 binding assay described herein.

| | | | | Cross- |
|---|---|---|---|---|
| | | RBA (R2) | RBA (R2) | reactivity |
| | | IC50 − | IC50 + | with mouse |
| | | protein L | protein L | VEGF in |
| Lead | dAb | (nM) | (nM) | ELISA |
| TAR15-1 | VK | 171 | 7.4 | + |
| TAR15-10 | VK | 12.2 | 0.3 | + |
| TAR15-16 | VK | 31 | 1.7 | +/− |
| TAR15-17 | VK | 38 | 0.5 | +/− |
| TAR15-18 | VK | 174 | 0.4 | + |
| TAR15-20 | VK | 28 | 0.3 | − |

The TAR15-1 clone has a Kd of 50-80 nM when tested at various concentrations on a low density BIAcore chip. Other VK clones were passed over the low density chip at one concentration (50 nM). Different clones show different kinetic profiles.

Amino acid sequences:

Consensus sequence: W28, G30, E32, S34, HSO and Y93.

Additional TAR15 anti-human VEGF dAb clones have a consensus sequence:

W28, G30, E32, S34, H50 and Y93, as shown in TAR15-10 below.

TAR15-1

(SEQ ID NO: 192)

DIQMTQSPSSLSASVGDRVTITCRASQWIGPELSWYQQKPGKAPKLLIYH
GSILQSGVPSRFSGSGSGTDFTLTISSLQPEDFATYYCQQRMYRPATFGQ
GTKVEIKR
TAR15-3

(SEQ ID NO: 193)

DIQMTQSPSSLSASVGDRVTITCRASQWIGRELKWYQQKPGKAPRLLIYH
GSVLQSGVPSRFSGSGSGTDFTLTISSLQPEDFATYYCQQDFFVPDTFGQ
GTKVEIKR
TAR15-4

(SEQ ID NO: 194)

DIQMTQSPSSLSASVGDRVTITCRASQDIANDLMWYQQKPGKAPKLLIYR
NSRLQGGVPSRFSGSGSGTDFTLTISSLQPEDFATYYCQQLVHRPYTIGQ
GTKVEIKR
TAR15-9

(SEQ ID NO: 195)

DIQMTQSPSSLSASVGDRVTITCRASQFIGPHLTWYQQKPGKAPKLLIYH
SSLLQSGVPSRFSGSGSGTDFTLTISSLQPEDFATYYCQQYMYYPSTFGQ
GTKVKIKR
TAR15-10

(SEQ ID NO: 196)

DIQMTQSPSSLSASVGDRVTITCRASQWIGPELSWYQQKPGKAPKLLIYH
TSILQSGVPSRFSGSGSGTDFTLTISSLQPEDFATYYCQQYMFQPRTFGQ
GTKVEIRR
TAR15-11

(SEQ ID NO: 197)

DIQMIQSPSSLSASVGDRVTITCRASQFIGNELSWYQQKPGKAPKLLIYH
ASSLQSGVPSRFSGSGSGTDFTLTISSLQPEDFATYYCQQVLGYPYTFGQ
GTKVEIKR
TAR15-12

(SEQ ID NO: 198)

DIQMTQSPSSLSASVGDRVTITCRASQWIGPELSWYQQKPGKAPKLLIYH
GSILQSGVPSRFSGSGSGTDFTLTISSLQPEDFATYYCQQVLYSPLTFGQ
GTKVEIKR
TAR15-13

(SEQ ID NO: 199)

DIQMTQSPSSLSASVGDRVTITCRASQWIGNELKWYQQKPGKAPKLLIYM
SSLLQSGVPSRFSGSGSGTDFTLTISSLQPEDLATYYCQQTLLLPFTFGQ
GTKVEIKR
TAR15-14

(SEQ ID NO: 200)

DIQMTQSPSSLSASVGDRVTITCRASQWIGPELSWYQQKPGKAPKLLIYH
GSILQSGVPSRFSGSGSGTDFTLTISSLQPEDFATYYCQQRLYYPGTFGQ
GTKVEIKR
TAR15-15

(SEQ ID NO: 201)

DIQMTQSPSSLSASVGDRVTITCRASQSIGRELSWYQQKPGKAPMLLIYH
SSNLQSGVPSRFSGSGSGTDFTLTISSLQPEDFATYYCQQGMYWPYTFGQ
GTKVEIKR
TAR15-16

(SEQ ID NO: 202)

DIQMTQSPSSLSASVGDRVTITCRASQWIKPALHWYQQKPGKAPKLLIYH
GSILQSGVPSRFSGSGSGTDFTLTISSLQPEDFATYYCQQTLFMPYTFGQ
GTKVEIKR
TAR15-17

(SEQ ID NO: 203)

DIQMTQSPSSLSASVGDRVTITCRASQSISTALLWYQQKPGKAPKLLIYN
GSMLPNGVPSRFSGSGSGTDFTLTISSLQPEDFATYYCQQTWDTPMTFGQ
GTKVEIKR
TAR15-18

(SEQ ID NO: 204)

DIQMTQSPSSLSASVGDRVTITCRASQWIGHDLSWYQQKPGKAPKLLIYH
SSSLQSGVPSRFSGSGSGTDFTLTISSLQPEDVATYYCQQLMGYPFTFGQ
GTKVEIKR
TAR15-19

(SEQ ID NO: 205)

DIQMTQSPSSLSASVGDRVTITCRASQDIGGLLVWYQQKPGKAPKLLIYR
SSYLQSGVPSRFSGSGSGTDFTLTISSLQPEDFATYYCQQTWGIPHTFGQ
GTKVEIKR
TAR15-20

(SEQ ID NO: 206)

DIQMTQSPSSLSASVGDRVTITCRASQKIFNGLSWYQQKPGKAPKLLIYH
SSTLQSGVPSRFSGSGSGTDFTLTISSLQPEDFATYYCQQVLLYPYTFGQ
GTKVEIKR
TAR 15-22

(SEQ ID NO: 207)

DIQMTQSPSSLSASVGDRVTITCRASQSIGTNLSWYQQKPGKAPRLLIYR
TSMLQSGVPSRFSGSGSGTDFTLTISSLQPEDFATYYCQQQFFWPHTFGQ
GTKVEIKR

VH dAbs that bind human VEGF are described below. These clones give a reduction (more than 50%) in the supernatant RBA (R2):

| | | More than 50% | |
|---|---|---|---|
| | | reduction in | Cross-reactivity |
| | | supernatant | with mouse |
| Lead | dAb | RBA (R2) | VEGF in ELISA |
| TAR15-5 | VH | + | + |
| TAR15-6 | VH | + | +/− |
| TAR15-7 | VH | + | +/− |
| TAR15-8 | VH | + | + |
| TAR15-23 | VH | + | − |
| TAR15-24 | VH | + | − |
| TAR15-25 | VH | + | − |
| TAR15-26* | VH | + | +/− |
| TAR15-27 | VH | + | +/− |
| TAR15-29 | VH | + | − |
| TAR15-30 | VH | + | − |

*TAR15-26, cross-linked using anti-Myc tag antibody, gives an IC₅₀ of 10 nM against mouse VEGF, and 3 nM against human VEGF.

VH clones were passed over the low density VEGF chip on a BIAcore at one concentration (50 nM). Different clones give different kinetic profiles.

Amino acid sequences:

TAR15-5

(SEQ ID NO: 208)

EVQLLESGGGLVQPGGSLRLSCAASGFTFRLYDMVWVRQAPGKGLEWVSY
ISSGGSGTYYADSVKGRFTISRDNSKNTLYLQMNSLRAEDTAVYYCAKAG
GRASFDYWGQGTLVTVSS
TAR15-6

(SEQ ID NO: 209)

EVQLLESGGGLVQPGGSLRLSCAASGFTFHLYDMMWVRQAPGKGLEWVSF
IGGDGLNTYYADSVKGRFTISRDNSKNTLYLQMNSLRAEDTAVYYCAKAG
TQFDYWGQGTLVTVSS
TAR15-7

(SEQ ID NO: 210)

EVQLLESGGGLVQPGGSLRLSCAASGFTFNKYPMMWVRQAPGKGLEWVSE
ISPSGQDTYYADSVKGRFTISRDNSKNTLYLQMNSLRAEDTAVYYCAKNP
QILSNFDYWGQGTLVTVSS
TAR15-8

(SEQ ID NO: 211)

EVQLLESGGGLVQPGGSLRLSCAASGFTFQWYPMWWVRQAPGKGLEWVSL
IEGQGDRTYYADSVKGRFTISRDNSKNTLYLQMNSLRAEDTAVYYCAKAG
DRTAGSRGNSFDYWGQGTLVTVSS
TAR15-23

(SEQ ID NO: 212)

EVQLLESGGGLVQPGGSLRLSCAASGFTFKAYEMGWVRQAPGKGLEWVSG
ISPNGGWTYYADSVKGRFTISRDNSKNTLYLQMNSLRAEDTAVYYCAKES
ISPTPLGFDYWGQGTLVTVSS
TAR15-24

(SEQ ID NO: 213)

EVQLLESGGGLVQPGGSLRLSCAASGFTFTGYEMGWVRQAPGKGLEWVSY
ISRGGRWTYYADSVKGRFTISRDNSKNTLYLQMNSLRAEDTAVYYCAKSD
TMFDYWGQGTLVTVSS
TAR15-25

(SEQ ID NO: 214)

EVQLLESGGGLVQPGGSLRLSCAASGFTFSAYEMGWVRQAPGKGLEWVSF
ISGGGRWTYYADSVKGRFTISRDNSKNTLYLQMNSLRAEDTAVYYCAKYS
EDFDYWGQGTLVTVSS
TAR15-26

(SEQ ID NO: 215)

EVQLLESGGGLVQPGGSLRLSCAASGFTFGAYPMMWVRQAPGKGLEWVSE
ISPSGSYTYYADSVKGRFTISRDNSKNTLYLQMNSLRAEDTAVYYCAKDP
RKFDYWGQGTLVTVSS
TAR15-27

(SEQ ID NO: 216)

EVQLLESGGGLVQPGGSLRLSCAASGFTFQFYKMGWVRQAPGKGLEWVSS
ISSVGDATYYADSVKGRFTISRDNSKNTLYLQMNSLRAEDTAVYYCAKMG
GGPPTYVVYFDYWGQGTLVTVSS
TAR15-29

(SEQ ID NO: 217)

EVQLLESGGGLVQPGGSLRLSCAASGFTFGEYGMYWVRQAPGKGLEWVSS
ISERGRLTYYADSVKGRFTISRDNSKNTLYLQMNNLRAEDTAVYYCAKSA
LSSEGFSRSFDYWGQGTLVTVSS
TAR15-30

(SEQ ID NO: 218)

EVQLLESGGGLVQPGGSLRLSCAASGFTFSDYAMYWVRQAPGKGLEWVSS
ITARGFITYYADSVKGRFTISRDNSKNTLYLQMNSLRAEDTAVYYCAKSG
FPHKSGSNYFDYWGQGTLVTVSS

### Example 30

### Additional Studies with Anti-VEGF dAbs

Anti VEGF dAbs as described herein can be tested for efficacy in various formats as described above for the anti-TNF-α dAbs, including, for example, Fc fusion, Fabs, PEGylated forms, dimers, tetramers, and anti-SA dual specific forms. The anti-VEGF dAbs can also be evaluated not only with anti-TNF-α dAbs as described herein, but also with other anti-TNF-α preparations, such as HUMIRA, ENBREL, and/or REMICADE.

The additional studies can be carried out to examine the effects of anti-VEGF dAbs on, for example, arthritic and histopathologic scores in the Tg197 model of RA.

For example, a TAR15 dAb Fc fusion similar to the TAR1-5-19 Fc fusions described herein above are administered IP at 1 mg/kg or 10 mg/kg, weekly or twice weekly commencing at 3 weeks of age (before the onset of RA symptoms), or at 6 weeks of age (after the onset of symptoms) and continuing for up to 7 weeks or more. Results are judged in comparison with saline, control Fc fusion (anti-β-gal), TAR1-5-19 alone, ENBREL, REMICADE and/or HUMIRA, preferably in equal molar amounts.

Animals are scored for macrophenotypic indicia (e.g., arthritic score) and histopathological scores as described above. Efficacy is demonstrated by any of

i) a failure to develop disease symptoms (as evidenced by arthritic or histopathological scores) when administered to animals beginning at 3 weeks of age,

ii) lessened severity of disease symptoms appearing when administered starting at 3 weeks of age, relative to control animals,

iii) failure to progress to more severe disease or progression at a lower rate relative to control animals when administered beginning at 6 weeks of age,

iv) reversal of symptoms (again, by arthritic score or hostopathological score) at any of 7, 8, 9, 10, 11, 12, or 14 weeks when administered to an animal beginning at 6 weeks of age.

Similar studies can be carried out with each of the different formats described above, e.g., Fabs, PEGylated forms, dimers, tetramers, and anti-SA dual specific forms.

Anti VEGF dAbs such as TAR15 dAb can also be administered to the Tg197 mouse model in combination with HUMIRA, ENBREL, and/or REMICADE. Such studies are performed in the same manner as described above for the testing of VEGF dAbs alone, and efficacy is also determined in the same manner.

### Example 31

### Evaluation of Anti-TNF-α dAbs in a Crohn's Disease Model

To evaluate the effectiveness of anti-TNF-α dAbs (and/or anti-VEGF dAbs) in Crohn's disease, the Tnf^{ΔARE} transgenic mouse model of Crohn's disease originally described by Kontoyiannis et al., 1999, Immunity 10: 387-398 is used (the DSS model can also be used in a similar fashion). The animals develop an IBD phenotype with similarity to Crohn's disease starting between 4 and 8 weeks of age. Therefore, anti-TNF-a dAb, e.g., TAR1-5-19 in various formats (Fc fusion, Fab, PEGylated (dimeric, tetrameric, etc.), dual specific with VEGF, dual specific with anti-SA, etc.) is administered at either 3 weeks of age (to test prevention of disease) or 6 weeks of age (to test stabilization, prevention of progression or reversal of disease symptoms), and animals are scored by weight and histologically as described herein. IP dosages of 1 mg/kg and 10 mg/kg are used for initial studies, with adjustments made in accord to the results of these initial studies. Test compositions are administered either weekly or twice weekly, or can be administered continuously, for example, using an osmotic pump. Alternatively, oral delivery formulations, e.g., by oral gavage with Zantac or by enteric coated formulations can also be applied. The studies are continued for up to 7 weeks or more once initiated.

Efficacy in the TNF^{ΔARE} model of Crohn's disease is shown by any of:

i) a failure to develop disease symptoms when administered to animals beginning at 3 weeks of age,

ii) lessened severity of disease symptoms appearing when administered starting at 3 weeks of age, relative to control animals,

iii) failure to progress to more severe disease or progression at a lower rate relative to control animals when administered beginning at 6 weeks of age,

iv) reversal of symptoms at any of 7, 8, 9, 10, 11, 12, or 14 weeks when administered to an animal beginning at 6 weeks of age.

In particular, treatment is considered effective if the average histopathological disease score is lower in treated animals (by a statistically significant amount) than that of a vehicle control group. Treatment is also considered effective if the average histopathological score is lower by at least 0.5 units, at least 1.0 unit, at least 1.5 units, at least 2.0 units, at least 2.5 units, at least 3.0 units, or by at least 3.5 units relative to the vehicle-only control group. Alternatively, the treatment is effective if the average histopatholigical score remains at or is lowered to 0 to 0.5 throughout the course of the therapeutic regimen

As with the RA model, the effect of combination therapies with dAbs specific for VEGF or with other anti-TNF-a compositions (e.g., ENBREL, REMICADE and/or HUMIRA) is also evaluated in this model.

### Example 32

### Dual-Specific IgG Directed Against Human TNF-Alpha and Human VEGF

In the engineered IgG-like dual-specific format described below, dAbs of two different specificities are fused to heavy and light chain constant domains, respectively. Upon co-expression in a cell, a two-armed IgG-like molecule is generated in which two variable domains capable of binding to two therapeutic targets (e.g., one specific for TNF-α and one specific for VEGF) are present on each arm of the dual targeting IgG.

DNA constructs. Mammalian expression vectors used were based on the Invitrogen pcDNA3.1 backbone, which facilitates gene expression in mammalian cells via the CMV immediate early promoter. For heavy chain expression, a cassette consisting of a human CD33 signal peptide and the human IgG1 heavy chain constant domain was inserted into the NheI and XbaI restriction sites of the vector pcDNA3.1(+), and variable domains specific for VEGF and expressed as part of the heavy chain polypeptide were cloned into this cassette between the CD33 signal peptide and the IgG1 heavy chain constant domain, using HindIII and NotI restriction sites. For light chain expression, a cassette consisting of a CD33 signal peptide and the human C kappa constant domain was inserted into the NheI and XhoI restriction sites of the vector pcDNA3.1zeo(+), and variable domains specific for TNF-alpha and expressed as part of the light chain polypeptide were cloned into this cassette between the CD33 signal peptide and the C kappa constant domain, using HindIII and NotI restriction sites.

Protein expression and purification. DNA of the heavy and light chain expression vectors was prepared using the Qiagen EndoFree plasmid Mega kit according to manufacturer's instructions, and used to transfect HEK293 (obtained from the European Collection of Cell Cultures) or Cos-7 cells (obtained from the American Type Culture Collection) with the Roche transfection reagent Fugene6, according to manufacturer's instructions. After 5 days, culture supernatants were harvested by centrifugation and secreted dual-specific antibodies were purified using two-step affinity purification. First, culture supernatants were supplemented with phosphate-buffered saline (PBS) to a final concentration of 1.5× PBS, and antibodies were captured on Amersham Streamline protein A resin. Resins were washed with 2× PBS, followed by 10 mM Tris pH8, and bound antibodies were eluted using 0.1M glycine pH2. Eluates were neutralised by adding a 25% volume of 1M Tris pH8, and recombinant antibodies were captured on Affitech protein L agarose resin. Resins were again washed using 2× PBS, followed by 10 mM Tris pH8, and bound recombinant antibodies were eluted using 0.1M glycine pH2 and eluates neutralised by adding a 25% volume of 1M Tris pH8.

Analysis of recombinant antibodies. The purified recombinant antibodies were quantified on a spectrophotometer using absorbance reading at 280 nm and analysed by SDS-PAGE, using Invitrogen NuPAGE 4-12% Bis-Tris gels and SilverQuest staining, according to manufacturer's instructions. FIG. 40 shows the SDS-PAGE analysis of a dual-specific antibody that comprises a kappa variable domain specific for human VEGF fused to the human IgG1 heavy chain constant domain and a kappa variable domain specific for human TNF-alpha fused to the human C kappa constant domain. Lane 1 was loaded with the Invitrogen MultiMark molecular weight marker, lane 2 was loaded with the dual-specific antibody in 1× Invitrogen NuPAGE LDS sample buffer and lane 3 with the dual-specific antibody in 1× Invitrogen NuPAGE LDS sample buffer supplemented with 10 mM betamercaptoethanol. In lane 3, the heavy chain is seen as a 50 kDa band and the light chain is seen as a 25 kDa band.

Testing of dual specificity. The dual-specific nature of the expressed antibodies was demonstrated by measuring the potency of each purified batch of antibody both in a human TNF-cell assay and in a human VEGF receptor binding assay.

The human TNF cell-based assay used was the L929 cytotoxicity assay described by Evans (2000, Molecular Biotechnology 15, 243-248). Briefly, L929 cells plated in microtitre plates were incubated overnight with dual-specific antibody, 100 pg/ml TNF and 1 mg/ml actinomycin D (Sigma, Poole, UK). Cell viability was measured by reading absorbance at 490nm following an incubation with [3-(4,5-dimethylthiazol-2-yl)-5-(3-carbboxymethoxyphenyl)-2-(4-sulfophenyl)-2H- tetrazolium (Promega, Madison, USA). Anti-TNF activity led to a decrease in TNF cytotoxicity and therefore an increase in absorbance compared with the TNF only control.

VEGF activity was measured using the VEGFR2 binding assay essentially as described above in the section titled “Preparation of immunoglogulin based multi-specific ligands.” Briefly, a 96 well Nunc Maxisorp assay plate was coated overnight with recombinant human VEGF R2/Fc (R&D Systems, Cat. No: 357-KD-050) at 0.5 μg/ml in carbonate buffer. Wells were washed repeatedly with 0.05% tween/PBS and then PBS. 2% BSA in PBS was added to block the plate. Wells were washed (as above), then purified dual-specific antibody was added to each well. VEGF, at 6 ng/ml in diluent (for a final concentration of 3 ng/ml), was then added to each well and the plate incubated for 2 hr at room temperature. Wells were washed as above, and then biotinylated anti-VEGF antibody (R&D Systems, Cat No: BAF293) at 0.5 μg/ml in diluent was added and incubated for 2 hr at room temperature. Wells were washed as above, followed by the addition of HRP conjugated anti-biotin antibody (1:5000 dilution in diluent; Stratech, Cat No: 200-032-096). The plate was then incubated for 1 hr at room temperature. The plate was washed as above, ensuring any traces of Tween-20 have been removed. For detection, 100 μl of SureBlue 1-Component TMB MicroWell Peroxidase solution was added to each well. The reaction is stopped by the addition of 1M hydrochloric acid, followed by reading OD₄₅₀ using a plate reader.

FIG. 41 shows the results for a dual-specific antibody that comprises a kappa variable domain specific for human VEGF fused to the human IgG1 heavy chain constant domain and a kappa variable domain specific for human TNF-alpha fused to the human C kappa constant domain. The dual-specific antibody (denoted anti-TNF-alpha x anti-VEGF) bound both human TNF-alpha and human VEGF. The antibody is bivalent for both targets: The ND50 for TNF-alpha is significantly lower (24 nM) than for the anti-TNF-alpha monomer (200 nM) that is fused as a variable domain to C kappa in the dual-specific molecule. The EC50 for VEGF is much lower (75 pM) than for the anti-VEGF monomer (12 nM, not shown) that is fused as a variable domain to the heavy chain constant domain in the dual-specific molecule, and also lower than for the anti-VEGF monomer oligomerised by protein L cross-linking (line with data points shown as squares, 990 pM).

The constructs of this embodiment are tetravalent, dual-specific antigen-binding polypeptide constructs comprising two copies of a V_{H} or V_{L} single domain antibody that binds a first epitope; and two copies of a V_{H} or V_{L} single domain antibody that binds a second epitope. Each of the two copies of the single domain antibody that binds the first epitope is fused to an IgG heavy chain constant domain, and each of the two copies of the single domain antibody that binds the second epitope is fused to a light chain constant domain.

Additional dual-specific, tetravalent polypeptide constructs similar to those described in this Example can be generated by one of skill in the art using, for example, other anti-TNF-α and anti-VEGF antibody sequences, e.g., any of those described herein. In other embodiments, C_{κ} or C_{λ} light chain constant domains can be used, and IgG heavy chain constant domains other than IgG1 can also be used. Of particular interest for use in the development into constructs of this sort are single domain anti-TNF-α antibody clones that prevent an increase in arthritic score when administered to a mouse of the Tg197 transgenic mouse model of arthritis as a dAb monomer, and single domain anti-VEGF antibody clones that prevent an increase in arthritic score when administered to a mouse of a collagen-induced arthritis mouse model as a dAb monomer. It is also preferred that the monomer of the single domain anti-TNF-α antibody clone neutralizes human TNF-α in the L929 cell cytotoxicity assay described herein, and that the monomer of the single domain anti-VEGF antibody clone antagonizes VEGF receptor binding in an assay of VEGF Receptor 2 binding as described herein. It is preferred that the single domain antibody clones used bind their respective epitopes with a K_{d} of <100 nM. It is also preferred that such dual-specific, tetravalent constructs bind the respective epitopes with a K_{d} of <100 nM and prevent an increase in arthritic score in either or both of the Tg197 and CIA models of arthritis described herein.

Such constructs can be used for the treatment of rheumatoid arthritis in a manner similar to the other constructs described herein, in terms of administration, dosage and monitoring of efficacy. The half-life of the construct can be modified as described herein above, e.g., by addition of a PEG moiety, and/or by further fusion of a binding moiety (e.g., a further single domain antibody) specific for a protein that increases circulating half-life, e.g., a serum protein such as HSA.

### Example 33

Selection and characterisation of dAbs for binding to serum albumin from a range of species.

dAbs against human serum albumin, mouse serum albumin and porcine serum albumin were selected as previously described for the anti-MSA dAbs except for the following modifications to the protocol: The phage libraries of synthetic V_{H} domains were the libraries 4G and 6G, which are based on a human V_{H}3 comprising the DP47 germ line gene and the J_{H}4 segment for the VH and a human Vκ1 comprising the DPK9 germ line gene and the Jκ1 segment for the Vκ. The libraries comprise 1×10¹⁰ individual clones. A subset of the V_{H} and V_{κ} libraries had been preselected for binding to generic ligands protein A and protein L respectively so that the majority of clones in the unselected libraries were functional. The sizes of the libraries shown above correspond to the sizes after preselection.

Two or three rounds of selection were performed on mouse, porcine and human serum albumin using subsets of the V_{H} and Vκ libraries separately. For each selection, antigen was either (i) coated on immunotube (nunc) in 4 ml of PBS at a concentration of 100 μg/ml, or (ii) bitotinylated and then used for soluble selection followed by capture on streptavidin beads or neutravidin beads. In each case, after the second or third round of selection, DNA from the selection was cloned into an expression vector for production of soluble dAb, and individual colonies were picked. Soluble dAb fragments were produced as described for scFv fragments by Harrison et al (Methods Enzymol. 1996;267:83-109) and for each selection, 96 soluble clones were tested for binding to a range of serum albumins.

Screening of clones for binding to serum albumins from a range of species was done using a BIACORE surface plasmon resonance instrument (Biacore AB). A CM-5 biacore chip was coated with serum albumin from different species at high density on each of flow cells 2 to 4. dAbs which exhibited binding to one or more serum albumins of interest were sequenced and expressed at a 50 ml scale, purified on protein L and then screened at a known concentration for binding to a panel of serum albumins on a CM-5 BIAcore chip coated with a low density of serum albumin on flow cells 2 to 4. Several dAbs which bind serum albumin from a range of different species were found, with the preferred candidates being listed, along with their binding profiles, in Table 7.

TABLE 7

| | | RSA | MSA | |
|---|---|---|---|---|
| | HSA (affinity | (affinity if | (affinity if | Cyno (affinity |
| | if measured) | measured) | measured) | if measured) |
| DOM7h-9 | Binds 200 nM | binds | binds | binds |
| DOM7h-10 | binds | ND | ND | ND |
| DOM7h-11 | binds | binds | binds | binds |
| DOM7h-12 | binds | ND | binds | binds |
| DOM7h-13 | binds | binds | binds | |
| DOM7h-14 | Binds | binds | Binds | Binds 123 nM |
| | 38 nM | | 27 nM | |

In this experiment, we have therefore isolated dAbs that bind HSA and albumin from one or more of a range of non-human species. For example, we found dAbs that bind (i) human and mouse, (ii) human and cynomolgus, (iii) human and rat and (iv) human, mouse, rat and cyno albumin.

### Example 34

Determination of the serum half-life in rat and cynomolgus monkey of serum albumin binding dAb/HA epitope tag or dAb/myc epitope tag fusion proteins and determination of serum half life.

Anti-cynomolgus serum albumin dAbs were expressed with C-terminal HA or myc tags in the periplasm of E. coli and purified using batch absorption to protein L-agarose affinity resin (Affitech, Norway) for Vk dAbs and batch absorption to protein A affinity resin for VH dAbs, followed by elution with glycine at pH 2.0. In order to determine serum half life, groups of 3 cynomolgus macaques were given a single i.v. injection at 2.5 mg/Kg of DOM7h-9, DOM7h-11 or DOM7h-14. Blood samples were obtained by serial bleeds from a femoral vein or artery over a 21 day period and serum prepared from each sample. Serum samples were analysed by sandwich ELISA using goat anti-HA (Abcam, Cambridge UK) or goat anti myc (Abcam, Cambridge UK) coated on an ELISA plate, followed by detection with protein L-HRP. Standard curves of known concentrations of dAb were set up in the presence of cynomolgus serum at the same concentration as for the experimental samples to ensure comparability with the test samples. Fitting a double exponential Modelling with a 2 compartment model (using kaleidograph software (Synergy software, PA, USA)) was used to calculate t1/2β, see Table 8.

Anti-rat serum albumin dAbs were expressed with C-terminal HA or myc tags in the periplasm of E. coli and purified using batch absorption to protein L-agarose affinity resin (Affitech, Norway) followed by elution with glycine at pH 2.0. dAbs were then labelled with ³H using the following method: One vial per protein was prepared: 300 μL of NSP was dispensed into the vial and the solvent removed under a gentle stream of nitrogen at ≦30° C. The residue was then re-suspended in DMSO (100 μL). An aliquot of protein solution (2.5 mL) was added to the DMSO solution and the mixture incubated for 60 minutes at room temperature. Exactly 2.5 ml of the solution was then be loaded onto a pre-equilibrated PD10 column (pre-equilibrated with 25 mL Phosphate buffered saline, PBS) and the eluate discarded. Phosphate buffered saline (PBS, 3.5 mL) will be added and the eluate collected. This provided a labelled protein solution at approximately 2 mg/mL. The specific activity of the material was determined and conditional on efficient labelling, the solution was used immediately or stored at −20° C. until required.

In order to determine serum half life, groups of 4 rats were given a single i.v. injection at 2.5 mg/Kg of DOM7h-9, DOM7h-11, DOM7h-13 or DOM7h-14. Blood samples were obtained from a tail vein over a 7 day period and plasma prepared. Levels of ³H were determined by liquid scintillation counting and concentration of labelled protein in each sample calculated according to the known specific activity of the protein administered at the start of the experiment. Fitting a double exponential Modelling with a 2 compartment model (using kaleidograph software (Synergy software, PA, USA)) was used to calculate t1/2β, see Table 8.

TABLE 8

| Agent | Scaffold | t½β (cyno) | t½β (rat) |
|---|---|---|---|
| DOM7h-9 | V_{κ} | 3.8 days | 66 hours |
| DOM7h-11 | V_{κ} | 5.2 days | 61 hours |
| DOM7h-13 | V_{κ} | not tested | 73 hours |
| DOM7h-14 | V_{κ} | 6.8 days | 56 hours |
| DOM7r-3 | V_{κ} | | 53 hours |
| DOM7r-16 | V_{κ} | | 43 hours |
| DOM7h-9 | V_{κ} | 3.8 days | 66 hours |
| DOM7h-11 | V_{κ} | 5.2 days | 61 hours |
| DOM7h-13 | V_{κ} | not tested | 73 hours |
| DOM7h-14 | V_{κ} | 6.8 days | 56 hours |
| DOM7r-3 | V_{κ} | | 53 hours |
| DOM7r-16 | V_{κ} | | 43 hours |

The half life of albumin in rat and cynomolgus monkey is 53 hours (determined experimentally) and 7-8 days (estimated) respectively. It can be seen from Table 8 that the half life of dAbs DOM7r-3, DOM7h-9, DOM7h-11, DOM7h-13 and DOM7h-14 in rat approach or are substantially the same as the half life of albumin in rat. Also, it can be seen that that the half life of dAbs DOM7h-11 and DOM7h-14 in cynomolgus approach or are substantially the same as the half life of albumin in cynomolgus. dAb DOM7h-14 has a half life in both rat and cynomolgus that is substantially the same as the half life of albumin in both species.

### Example 35

### Epitope Mapping

The three domains of human serum albumin have previously been expressed in Pichia pastoris (Dockal Carter and Ruker (1999) J. Biol. Chem. 2000 Feb. 4;275(5):3042-50. We expressed the same domains using the Pichia pastoris pPICZaA vector and where required purified them to homogeneity on Mimetic Blue SA matrix (supplier: Prometic Biosciences) FIG. 42. The identification of the serum albumin domain bound by dAbs was assessed by one of two methods, immunoprecipitation of domain antibodies and by competition BIAcore. Results are shown below in FIG. 43 and FIG. 44.

For immunoprecipitation assay, 1 ml of Pichia pastoris supernatant expressing either HSA domain I, II or III was adjusted to pH7.4, and mixed with 1 μg dAb, and 10 μl of Protein A or Protein L agarose (for V_{H} or V_{K} dAbs respectively). The mixture was mixed by inversion for 1 hour to allow complex formation, then the agarose bound complex was recovered by centrifugation at 13,000×g for 10 minutes, the supernatant decanted, and the pelleted material washed once with PBS, and recovered by centrifugation. The beads were then resuspended in SDS-PAGE loading buffer containing dithiothreitol (DTT), heated to 70° C. for 10 minutes, then run on a 4-12% NuPAGE SDS-PAGE gels (supplier: Invitrogen), and stained with SimplyBlue safestain.

For competition BIAcore assay, purified dAbs were made up to 1 μM in HBS-EP at pH7.4, or 1 μM in 50 mM citrate phosphate buffer, 150 mM NaCl, pH5.0, and where required, with 7 μM purified HSA domain. BIAcore runs were carried out at a flow rate of 30 μl min over a CM5 chip surface coated with 500-1000 RU of human serum albumin, and a blank reference cell used to do baseline subtraction.

Table 9 provides a list of dAbs specific for human serum albumin and the domain(s) of human serum albumin to which they map (as determined by immunoprecipitation and/or BIAcore):

TABLE 9

| Clone | H/K | Mapped HSA domain |
|---|---|---|
| DOM7h-1 | K | Domain II |
| DOM7h-2 | K | Nd |
| DOM7h-6 | K | Nd |
| DOM7h-7 | K | Nd |
| DOM7h-8 | K | Domain II |
| DOM7h-9 | K | Domain II |
| DOM7h-10 | K | Nd |
| DOM7h-11 | K | Domain II |
| DOM7h-12 | K | Domain II |
| DOM7h-13 | K | Domain II |
| DOM7h-14 | K | Domain II |
| DOM7h-21 | H | Nd |
| DOM7h-22 | H | Domain I + III |
| DOM7h-23 | H | Nd |
| DOM7h-24 | H | Nd |
| DOM7h-25 | H | Nd |
| DOM7h-26 | H | Nd |
| DOM7h-27 | H | Domain III |
| DOM7h-30 | H | Domain III |
| DOM7h-31 | H | Nd |

Nd: not determined

In conclusion, the majority of dAbs bind to the 2^{nd} domain of HSA and are therefore not expected to compete with binding of human serum albumin to FcRn. Two dAbs (DOM7h-27 and DOM7h-30) bind to Domain III.

| | HSA | | RU HSA | |
|---|---|---|---|---|
| | domain | RU HSA binding at | binding at | |
| dAb | bound | 1 μM pH7.4 | 1 μM pH5.0 | His in CDR |
| DOM7h-1 | II | 600c | 150 | no |
| DOM7h-3 | NI | 0 | 0 | |
| DOM7h-4 | NI | 0 | 0 | |
| DOM7h-8 | II | 1000 | 250 | no |
| DOM7h-9 | II | 150 | 0 | CDR1 |
| DOM7h-11 | II | 250 | 0 | CDR3 |
| DOM7h-12 | IIa | 55 | 0 | no |
| DOM7h-13 | II | 300 | 40 | 2 in CDR3 |
| DOM7h-14 | II | 20 | 0 | no |
| DOM7h-22 | I + IIIb | 100c | 0 | CDR2 |
| DOM7h-27 | III | 50 | 0 | no |
| DOM7h-30 | III | 320 | 35 | no |

Summary of results of epitope mapping of HSA binding AlbudAb™s (dAbs which specifically binds serum albumin) and Biacore data at pH7.4 and 5.0.

### Example 36

### Selecting dAbs In Vitro in the Presence of Metabolites

Albumin molecules accumulate the effects of exposure to other compounds in serum during their lifetime of around 19 days. These effects include the binding of numerous molecules that have affinity for albumin which include but are preferably not limited to cysteine and glutathione carried as mixed disulphides, vitamin B₆, δ-bilurubin, hemin, thyroxine, long and medium, chain fatty acids and glucose carried on ε-amino groups. Also, metabolites such as acetaldehyde (a product of ethanol metabolism in the liver), fatty acid metabolites, acyl glucuronide and metabolites of bilirubin. In addition, many drugs such as warfarin, halothane, salicylate, benzodiazepines and others (reviewed in Fasano et al 2005, IUBMB Life)) and also 1-O-gemfibrozil-β-D-glucuronide bind to serum albumin.

Compounds found bound to serum albumin tend to bind at certain sites on the albumin molecule, thereby potentially blocking these sites for the binding of other molecules such as AlbudAbs™ (a dAb which specifically binds serum albumin). The binding sites for many ligands has been identified, the main and most well characterised binding sites are termed “Sudlow site 1” and “Sudlow site 2”. According to this nomenclature, Site 1 is located in sub-domain IIA, and binds warfarin and other drugs which generally are bulky, heterocyclic anionic molecules. Site 2 is located in sub domain IIIA, and binds aromatic carboxylic acids with an extended conformation, with the negative charge towards one end, such as the stereotypical site 2 ligand, ibuprofen. Secondary binding sites for both Warfarin and ibuprofen have been identified on domains II and I respectively. Other binding sites and sub-sites of these also exist, meaning that in the circulation, serum albumin exists with a complex mixture of bound ligands, with affinities that vary from 1×10⁻²M to 1×10⁻⁸M. Oleic acid for example binds to up 7 sites on SA (J Mol Biol. 2001;314:955-60).

Human serum albumin has been in crystallized complex with fatty acids (Petitpas I, Grune T, Bhattacharya A A, Curry S. Nat. Struct Biol. (1998) 5: 827-35). The binding sites for these molecules are situated in hydrophobic clefts around the SA surface, with an asymmetric distribution, despite the near three-fold symmetry of the HSA molecule. Later, the use of various recombinant fragments of serum albumin has aided more precise assignment of the contribution of the domains to formation of the binding sites (for example: Protein Sci (2000) 9:1455-65; J Biol Chem. (1999) 274:2930310). Displacement of bound ligands from SA plays an important role in drug interactions, for example the half life of warfarin is reduced as it is displaced from SA by ethanol (J Biol Chem. (2000) 275:38731-8). Other drugs affinity for SA is modified by the presence of other drugs in other binding sites. For example, diazepam binding to site 2 increases the affinity of site 1 for tenoxicam, as a result of conformational changes on binding. This significantly affects the pharmacokinetic properties (Fundam Clin Pharmacol. (1989) 3:267-79).

Thus, for a SA binding AlbudAb™ (a dAb which specifically binds serum albumin), it is desirable to select one that does not alter the binding characteristics of serum albumin for drugs bound to SA. Additionally, where drug binding has been shown to alter the conformation of SA, it is desirable to have an AlbudAb™ (a dAb which specifically binds serum albumin) that binds SA in both in the presence or absence of the drug. These approaches mean that it will be possible to identify an AlbudAb™ (a dAb which specifically binds serum albumin) such that there are not significant positive or negative drug interactions with key pharmaceuticals. Therefore, this example describes a phage selection to identify dAbs that bind serum albumin in the presence of compounds and metabolites likely to be present bound to albumin in vivo. Phage selections are performed in the presence of one or several of the metabolites or compounds known to interact with serum albumin in vivo. These selections identify AlbudAb™s (a dAb which specifically binds serum albumin) that will bind to serum albumin in a manner that is unlikely to be hindered by the presence of metabolites or other compounds.

The phage libraries described in Example 1 are used as described in Example 1 for selection against albumin from one or more of a range of species including human, cynomolgus monkey, rat and mouse. The albumin used as an antigen is different from that described in Example 1 in that it will be preincubated overnight with ametabolite or compound at a 10-100 fold higher concentration than the albumin itself. This can either be with a single compound or metabolite, or with more than one compound or metabolite. In particular, it can be in the presence of compounds occupying albumin site I or site II or both. This concentration of metabolite is also present in the buffer used to coat the immunotubes with antigen and in the buffers used during key steps of the selection. Steps where metabolites are present include the MPBS blocking buffer used to block the antigen coated immunotubes or the biotinylated antigen (for solution selections) and also the buffer in which the phage library is blocked. In this way, when the blocked phage are added to the immunotube or biotinylated antigen, the concentration of metabolite is maintained. Therefore, throughout the phases of the selection in which the phage that bind to albumin are selected, metabolites that may block certain sites on the albumin molecule in vivo are also present, competing with the phage for binding and biasing the selection in favour of those dAbs that bind sites on albumin different from those blocked by metabolites.

In another set of selections, alternating rounds of selection against serum albumin in the presence and absence of bound compounds or metabolites are performed. This ensures that dAbs able to bind serum albumin in both the presence and absence of bound compounds are selected. In both selection schemes, it is possible that dAbs that are capable of displacing drug bound to serum albumin will be selected, and this is screened for by measuring the ability of the AlbudAb™ (a dAb which specifically binds serum albumin) to displace SA bound drug. Such assays are well established for small molecule drugs, and easily adapted for this purpose. A variety of methods well known in the art may be used to determine the ability of an AlbudAb™ (a dAb which specifically binds serum albumin) to displace SA bound drugs. These range from equilibrium dialysis, chromatographic methods on immobilised ligands or serum albumin, through NMR analysis. The following example describes the use of the simplest equilibrium dialysis method. The other more technically complex methods will give essentially the same information.

A solution of serum albumin is made at a defined concentration in a physiological buffer, for example, 20 mM sodium phosphate buffer, 150 mM NaCl, pH7.4. The drug is made up in a similar buffer, and has been synthesised such that it retains its original pharmacological properties, but is radiolabelled, for example with tritium or ¹⁴C. The serum albumin binding antibody fragment is made up at a defined concentration in a similar buffer.

The serum albumin solution is placed in a series of tubes, and increasing amount of AlbudAb™ (a dAb which specifically binds serum albumin) is added, such that the concentration of serum albumin in each tube is fixed (for example at 1% w/v, approx 150 μM), while the (a dAb which specifically binds serum albumin)™ concentration ranges from 0 to 150 μM over the tube series. This comprises one experimental set.

A dialysis tube or container containing a fixed concentration of the radiolabelled ligand for each set is added to the tube. A concentration range from 0.2 to 10 mM may be suitable, depending on the ligand used, its affinity and solubility.

The cut-off size of the membrane used for dialysis should be such that the serum albumin and AlbudAb™ (a dAb which specifically binds serum albumin) do not diffuse through, but the radiolabelled ligand can diffuse freely. A cut off size of 3.5 Kda is sufficient for this purpose.

The mixture is stirred at a fixed temperature, for example 37° C., for a fixed period of time, to allow equilibrium of the radiolabelled drug between both compartments, for example, 5 hours. After this time, equilibrium should be attained which is influenced by the ability of the AlbudAb™ (a dAb which specifically binds serum albumin) binding the serum albumin to inhibit drug binding.

Both compartments are samples, and the radioactivity counted, using a scintillation counter. The concentration of albumin bound ligand can be determined by the difference in counts between the two compartments. The stoichiometric binding constant K′ can be calculated from the equilibrium concentration of bound ligand, b, free ligand, c, and albumin, p, in accordance with the equation K′=b/c(p−b). This assumes the binding of 1 molecule of ligand to one molecule of serum albumin.

Binding data can then be measured using a Scatchard plot in accordance with the equation r/c=nk−rk, where r is the fraction of albumin to which ligand is bound (i.e. b/p, and n is the number of binding sites per albumin molecule, and k is the site association constant. Values of n and k can be determined from plots of r/c against r.

Where the binding of an AlbudAb™ (a dAb which specifically binds serum albumin) blocks radiolabelled ligand binding, this will affect both the stoichiometric binding constant of the ligand, and also the apparent number of binding sites for the ligand. It may be predicted that as the AlbudAb™ (a dAb which specifically binds serum albumin) will bind at one defined site on the surface of serum albumin, and some ligands have more than one binding site on serum albumin, that not all binding sites will be blocked. In the situation where The AlbudAb™ (a dAb which specifically binds serum albumin) specifically binds to drug complexed serum albumin and displaces it, and the drug has a low therapeutic index and is serum bound, then a cut-off affinity for distinguishing between an AlbudAb™ (a dAb which specifically binds serum albumin) able to displace serum albumin bound to the drug from an AlbudAb™ (a dAb which specifically binds serum albumin) not able to displace serum albumin to drug, would range from 10 nM to 100 nM. This method is exemplified in the following paper: Livesey and Lund Biochem J. 204(1): 265-272 Binding of branched-chain 2-oxo acids to bovine serum albumin.

### Example 37

### Generation of Dual-Specific Ligand Comprising a Serum Albumin-Binding CTLA-4 Non-Immunoglobulin Scaffold via CDR Grafting

The CDR domains of dAb7h14 are used to construct a cytotoxic T-lymphocyte associated antigen 4 (CTLA-4) non-immunoglobulin scaffold polypeptide that binds human serum albumin in the following manner. The CDR1 (RASQWIGSQLS; SEQ ID NO.:______), CDR2 (WRSSLQS; SEQ ID NO.:______), and CDR3 (AQGAALPRT ; SEQ ID NO.:______) sequences of dAb7h14 are grafted into a soluble truncated mutant of CTLA-4 comprising the CTLA-4 V-like domain (as described in WO 99/45110; optionally, an engineered form of CTLA-4, e.g., in which A2 and A3 domains are deleted) in replacement of native CTLA-4 amino acid residues corresponding to CDR1 (SPGKATE; SEQ ID NO.:______) within the S1-S2 loop (the BC loop), CDR2 (YMMGNELTF; SEQ ID NO.:______), and CDR3 (LMYPPPYYL; SEQ ID NO.:______) within the S5-S6 loop, respectively (for details of the CTLA-4 scaffold composition and/or structure refer to WO 00/60070; WO 99/45110; Metzler et al. Nat. Struct. Biol. 4: 527-53; and Nuttall et al. Proteins Struct. Funct. Genet. 36:217-27, all incorporated herein by reference in their entirety). Expression of this CLTA-4-derived polypeptide in a pGC-, pPOW-based, or other art-recognized expression system is performed, with the anticipated production of predominantly monomeric soluble protein. Protein solubility of this CTLA-4-derived polypeptide is examined, and is anticipated to be superior to native extracellular CTLA-4 polypeptide. ELISA analysis is used to examine whether purified monomeric polypeptide specifically binds human serum albumin compared to non-specific antigens and compared to extracellular CTLA-4-derived polypeptides grafted with non-specific polypeptides (e.g., somatostatin substituted within the CDR1 loop structure). Real-time binding analysis by BIAcore is performed to assess whether human serum albumin specifically binds to immobilized CTLA-4-derived polypeptide comprising the anti-human serum albumin CDR domains of dAb7h14. (One of skill in the art will recognize that binding affinity can be assessed using any appropriate method, including, e.g., precipitation of labeled human serum albumin, competitive BIAcore assay, etc.) Optionally, expression of the CTLA-4 anti-human serum albumin polypeptide is enhanced via adjustment of the coding sequence using splice overlap PCR to incorporate codons preferential for E. coli expression. If no or low human serum albumin affinity (e.g., Kd values in the μM range or higher) is detected, at least one of a number of strategies is employed to improve the human serum albumin binding properties of the CDR-grafted CTLA-4 polypeptide, including any of the following methods that contribute to binding affinity.

Human serum albumin binding of CDR-grafted CTLA-4 polypeptide(s) presenting dAb7h14 CDRs is optimized via mutagenesis, optionally in combination with parallel and/or iterative selection methods as described below and/or as otherwise known in the art. CTLA-4 scaffold polypeptide domains surrounding grafted dAb7h14 CDR polypeptide sequences are subjected to randomized and/or NNK mutagenesis, performed as described infra. Such mutagenesis is performed within the CTLA-4 polypeptide sequence upon non-CDR amino acid residues, for the purpose of creating new or improved human serum albumin-binding polypeptides. Optionally, dAb7h14 CDR polypeptide domains presented within the CDR-grafted CTLA-4 polypeptide are subjected to mutagenesis via, e.g., random mutagenesis, NNK mutagenesis, look-through mutagenesis and/or other art-recognized method. PCR is optionally used to perform such methods of mutagenesis, resulting in the generation of sequence diversity across targeted sequences within the CDR-grafted CTLA-4 polypeptides. Such approaches are similar to those described infra for dAb library generation. In addition to random and/or look-through methods of mutagenesis, directed mutagenesis of targeted amino acid residues is employed where structural information establishes specific amino acid residues to be critical to binding of human serum albumin.

CTLA-4 polypeptides comprising grafted dAb7h14 CDR sequences engineered as described above are subjected to parallel and/or iterative selection methods to identify those CTLA-4 polypeptides that are optimized for human serum albumin binding. For example, following production of a library of dAb7h14 CDR-grafted CTLA-4 polypeptide sequences, this library of such polypeptides is displayed on phage and subjected to multiple rounds of selection requiring serum albumin binding and/or proliferation, as is described infra for selection of serum albumin-binding dAbs from libraries of dAbs. Optionally, selection is performed against serum albumin immobilized on immunotubes or against biotinlyated serum albumin in solution. Optionally, binding affinity is determined using surface plasmon resonance (SPR) and the BIAcore (Karlsson et al., 1991), using a BIAcore system (Uppsala, Sweden), with fully optimized CTLA-4-derived polypeptides ideally achieving human serum albumin binding affinity Kd values in the nM range or better.

Following identification of CTLA-4-derived polypeptides that bind human serum albumin, such polypeptides are then used to generate dual-specific ligand compositions by any of the methods described infra.

### Example 38

### Generation of Dual-Specific Ligand Comprising a Serum Albumin-Binding CTLA-4 Non-Immunoglobulin Scaffold via Selection of Serum Albumin Binding Moieties

A soluble truncated mutant of CTLA-4 comprising the native CTLA-4 V-like domain (as described in WO 99/45110; optionally, an engineered form of CTLA-4, e.g., in which A2 and A3 domains are deleted) and which has been engineered to contain regions(s) of variability, are displayed in a library and subjected to selection and, optionally, affinity maturation techniques in order to produce human serum albumin-binding CTLA-4 non-immunoglobulin scaffold molecules for use in the ligands of the invention.

Expression of this CLTA-4-derived polypeptide in a pGC-, pPOW-based, or other art-recognized expression system is performed. Protein solubility of this CTLA-4-derived polypeptide is examined, and mutagenesis is performed to enhance solubility of CTLA-4-derived polypeptide(s) relative to that of a native extracellular CTLA-4 polypeptide. ELISA analysis is used to examine whether purified monomeric polypeptide optionally specifically binds human serum albumin compared to non-specific single variable domains comprising a CTLA-4 derived scaffold, and compared to extracellular CTLA-4-derived polypeptides grafted with non-specific polypeptides (e.g., CTLA-4 polypeptide with somatostatin substituted within the CDR1 loop structure). Real-time binding analysis by BIAcore is performed to assess whether human serum albumin specifically binds to immobilized CTLA-4-derived polypeptide. Optionally, expression of the CTLA-4 anti-human serum albumin polypeptide is enhanced via adjustment of the coding sequence using splice overlap PCR to incorporate codons preferential for E. coli expression. Following detection of no or low binding affinity (e.g., Kd values in the □M range or higher) of a CTLA-4 polypeptide for human serum albumin, at least one of a number of strategies is employed to impart human serum albumin binding properties to the CTLA-4 polypeptide, including one or more of the following methods that contribute to binding affinity.

Human serum albumin binding of CTLA-4 scaffold polypeptide(s) is achieved and optimized via mutagenic methods, optionally in combination with parallel and/or iterative selection methods as described below and/or as otherwise known in the art. CTLA-4 polypeptide domains are subjected to randomized and/or NNK mutagenesis, performed as described infra. Such mutagenesis is performed upon the entirety of the CTLA-4 polypeptide or upon specific sequences within the CTLA-4 polypeptide, optionally targeting CDR-corresponding amino acids (e.g., CDR1 and/or CDR3 sequences are randomized, and resulting polypeptides are subjected to selection, e.g., as described in Example 6 of WO 99/45110). Optionally, specific amino acid residues determined or predicted to be structurally important to CDR-like loop presentation are targeted for mutagenesis. Mutagenesis, especially randomized mutagenesis, is performed in order to evolve new or improved human serum albumin-binding polypeptides. PCR is optionally used to perform such methods of mutagenesis, resulting in the generation of sequence diversity across targeted sequences within the CTLA-4 polypeptides. (Such approaches are similar to those described infra for dAb library generation.) In addition to random methods of mutagenesis, directed mutagenesis of targeted amino acid residues is employed where structural information establishes specific amino acid residues of CTLA-4 polypeptides to be critical to binding of human serum albumin.

CTLA-4 polypeptides engineered as described above are subjected to parallel and/or iterative selection methods to identify those CTLA-4 polypeptides that are optimized for human serum albumin binding. For example, following production of a library of mutagenized CTLA-4 polypeptide sequences, said library of polypeptides is displayed on phage and subjected to multiple rounds of selection requiring serum albumin binding and/or proliferation, as is described infra for selection of serum albumin-binding dAbs from libraries of dAbs. Optionally, selection is performed against serum albumin immobilized on immunotubes or against biotinlyated serum albumin in solution. Optionally, binding affinity is determined using surface plasmon resonance (SPR) and the BIAcore (Karlsson et al., 1991), using a BIAcore system (Uppsala, Sweden), with fully optimized CTLA-4-derived polypeptides ideally achieving human serum albumin binding affinity Kd values in the nM range or better.

Following identification of CTLA-4 polypeptides that bind human serum albumin, such polypeptides are then used to generate dual-specific ligand compositions by any of the methods described infra.

CTLA-4 V-Like Domains

CTLA-4 is an example of a non-immunoglobulin ligand that binds to a specific binding partner and also comprises V-like domains. These V-like domains are distinguished from those of antibodies or T-cell receptors because they have no propensity to join together into Fv-type molecules. Such a non-immunoglobulin ligand provides an alternative framework for the development of novel binding moieties with high affinities for target molecules. Single domain V-like binding molecules derived from CTLA-4 which are soluble are therefore desirable.

Cytotoxic T-lymphocyte associated antigen 4 (CTLA-4) is involved in T-cell regulation during the immune response. CTLA-4 is a 44 Kda homodimer expressed primarily and transiently on the surface of activated T-cells, where it interacts with CD80 and CD86 surface antigens on antigen presenting cells to effect regulation of the immune response (Waterhouse et al. 1996 Immunol Rev 153: 183-207, van der Merwe et al. 1997 J Exp

Med 185: 393-403). Each CTLA-4 monomeric subunit consists of an N-terminal extracellular domain, transmembrane region and C-terminal intracellular domain. The extracellular domain comprises an N-terminal V-like domain (VLD; of approximately 14 Kda predicted molecular weight by homology to the immunoglobulin superfamily) and a stalk of about 10 residues connecting the VLD to the transmembrane region. The VLD comprises surface loops corresponding to CDR-1, CDR-2 and CDR-3 of an antibody V-domain (Metzler et al. 1997 Nat Struct Biol 4: 527-531). Recent structural and mutational studies on CTLA-4 indicate that binding to CD80 and CD86 occurs via the VLD surface formed from A′GFCC′ V-like beta-strands and also from the highly conserved MYPPPY sequence in the CDR3-like surface loop (Peach et al. 1994 J Exp Med 180: 2049-2058; Morton et al. 1996 J. Immunol. 156: 1047-1054; Metzler et al. 1997 Nat Struct Biol 4: 527-531). Dimerisation between CTLA-4 monomers occurs through a disulphide bond between cysteine residues (CYS120) in the two stalks, which results in tethering of the two extracellular domains, but without any apparent direct association between V-like domains (Metzler et al. 1997 Nat Struct Biol 4: 527-531).

Replacement of CDR loop structures within the VLDs has previously been shown to result in the production of monomeric, correctly folded molecules with altered binding specificities and improved solubility. Accordingly, in certain embodiments, a binding moiety comprising at least one monomeric V-like domain (VLD) derived from CTLA-4 is generated, wherein the at least one monomeric V-like domain is characterized in that at least one CDR loop structure or part thereof is modified or replaced such that the solubility of the modified VLD is improved when compared with the unmodified VLD.

In certain embodiments, at least one CDR loop structure or part thereof is modified or replaced such that (i) the size of the CDR loop structure is increased when compared with corresponding CDR loop structure in the unmodified VLD; and/or (ii) the modification or replacement results in the formation of a disulphide bond within or between one or more of the CDR loop structures.

In certain embodiments, the present invention provides a binding moiety comprising at least one monomeric V-like domain (VLD) derived from CTLA-4, the at least one monomeric V-like domain being characterized in that at least one CDR loop structure or part thereof is modified or replaced such that (i) the size of the CDR loop structure is altered when compared with corresponding CDR loop structure in the unmodified VLD; and/or (ii) the modification or replacement results in the formation of a disulphide bond within or between one or more of the CDR loop structures.

In certain embodiments, the size of the CDR loop structure is increased by at least two, more preferably at least three, more preferably at least six and more preferably at least nine amino acid residues. In further embodiments, the modified binding moiety of the invention also exhibits an altered binding affinity or specificity when compared with the unmodified binding moiety. Preferably, the effect of replacing or modifying the CDR loop structure is to reduce or abolish the affinity of the VLD to one or more natural ligands of the unmodified VLD. Preferably, the effect of replacing or modifying the CDR loop structure is also to change the binding specificity of the VLD (e.g., to produce a composition that binds human serum albumin). Thus, it is preferred that the modified VLD binds to a specific binding partner (e.g., human serum albumin) that is different to that of the unmodified VLD.

The phrase “VLD” is intended to refer to a domain which has similar structural features to the variable heavy (VH) or variable light (VL) antibody. These similar structural features include CDR loop structures.

As used herein, the term “CDR loop structures” refers to surface polypeptide loop structures or regions like the complementarity determining regions in antibody V-domains.

It will be appreciated that the CTLA-4-derived binding moieties of the present invention may be coupled together, either chemically or genetically, to form multivalent or multifunctional reagents. For example, the addition of C-terminal tails, such as in the native CTLA-4 with Cys′20, will result in a dimer. The binding moieties of the present invention may also be coupled to other molecules for various formulations, including those comprising dual specific ligands. For example, the CTLA-4 VLDs may comprise a C-terminal polypeptide tail or may be coupled to streptavidin or biotin. The CTLA-4 VLDs may also be coupled to radioisotopes, dye markers or other imaging reagents for in vivo detection and/or localization of cancers, blood clots, etc. The CTLA-4 VLDs may also be immobilized by coupling onto insoluble devices and platforms for diagnostic and biosensor applications.

In certain embodiments of the present invention, the extracellular CTLA-4 V-like domain is used. One or more surface loops of the CTLA-4 V-like domain and preferably the CDR1, CDR2 or CDR3 loop structures are replaced with a polypeptide which has a binding affinity for serum albumin (e.g., CDR domains of dAb7h14 and sequences derived therefrom, as exemplified infra). It will be appreciated that these CTLA-4 VLDs may be polyspecific, having affinities directed by both their natural surfaces and modified polypeptide loops.

One or more of the CDR loop structures of the CTLA-4 VLD can be replaced with one or more CDR loop structures derived from an antibody. The antibody may be derived from any species. In a preferred embodiment, the antibody is derived from a human, rat, mouse, camel, llama or shark. The CDR1 and CDR3 loop structures may adopt non-canonical conformations which are extremely heterologous in length. The V-like domain may also possess a disulphide linkage interconnecting the CDR1 and CDR3 loop structures (as found in some camel VHH antibodies) or the CDR2 and CDR3 loop structures (as found in some llama VHH antibodies).

For in vivo applications it is preferable that VLDs are homologous to the subject of treatment or diagnosis and that any possible xenoantigens are removed. Accordingly, it is preferred that VLD molecules for use in clinical applications are substantially homologous to naturally occurring human immunoglobulin superfamily members.

Serum albumin binding of CTLA-4 polypeptides (e.g., VLDs derived from CTLA-4) can be optimized via selection of a binding moiety with an affinity for serum albumin, e.g., comprising screening a library of polynucleotides for expression of a binding moiety with an affinity for serum albumin, wherein the polynucleotides have been subjected to mutagenesis which results in a modification or replacement in at least one CDR loop structure in at least one VLD and wherein the solubility of the isolated modified VLD is improved when compared with the isolated unmodified VLD.

It will be appreciated by those skilled in the art that within the context of such affinity screening method, any method of random or targeted mutagenesis may be used to introduce modifications into the V-like domains. In a preferred embodiment, the mutagenesis is targeted mutagenesis. Optionally, the targeted mutagenesis involves replacement of at least one sequence within at least one CDR loop structure using, e.g., splice overlap or other PCR technology.

It will also be appreciated by those skilled in the art that the polynucleotide library may contain sequences which encode VLDs comprising CDR loop structures which are substantially identical to CDR loop structures found in naturally occurring immunoglobulins and/or sequences which encode VLDs comprising non-naturally occurring CDR loop structures. Optionally, the screening process involves displaying the modified V-like domains as gene III protein fusions on the surface of bacteriophage particles.

The library may comprise bacteriophage vectors such as pHFA, fd-tet-dog or pFAB.5c containing the polynucleotides encoding the V-like domains. The screening process can also involve displaying the modified V-like domains in a ribosomal display selection system.

The preferred CTLA-4-derived serum albumin binding molecules of the present invention provide the following advantages (i) use of a native human protein obviates the need for subsequent humanization of the recombinant molecule, a step often required to protect against immune system response if used in human treatment; (ii) the domain is naturally monomeric as described above (incorporation of residue Cys120 in a C-terminal tail results in production of a dimeric molecule); and (iii) structural modifications have resulted in improved E. coli expression levels.

Initial determination of native CTLA-4 structure allowed modeling and prediction of the regions corresponding to antibody CDR1, 2 and 3 regions. It was hypothesized that such areas would be susceptible to mutation or substitution without substantial effect upon the molecular framework and hence would allow expression of a correctly folded molecule. The published structure of CTLA-4 (Metzler et al. 1997 Nat Struct Biol 4: 527-531) showed these predictions to be accurate, despite the unexpected separation of CDR1 from the ligand-binding site, and the extensive bending of CDR3 to form a planar surface contiguous with the ligand binding face.

V-like domains provide a basic framework for constructing soluble, single domain molecules, where the binding specificity of the molecule may be engineered by modification of the CDR loop structures. The basic framework residues of the V-like domain may be modified in accordance with structural features present in camelid antibodies. The camel heavy chain immunoglobulins differ from “conventional” antibody structures by consisting of VHH chains, (Hamers-Casterman et al. 1993 Nature 363: 446-448). Cammelid antibldies consist of two heavy chains, each comprising a VHH domain. Several unique features allow these antibodies to overcome the dual problems of solubility and inability to present a sufficiently large antigen binding surface.

First, several non-conventional substitutions (predominantly hydrophobic to polar in nature) at exposed framework residues reduce the hydrophobic surface, while maintaining the internal beta-sheet framework structure (Desmyter et al. 1996 Nat Struct Biol 3:803-811). Further, within the three CDR loops several structural features compensate for the loss of antigen binding-surface usually provided by the VL domain. While the CDR2 loop does not differ extensively from other VH domains, the CDR1 and CDR3 loops adopt non-canonical conformations which are extremely heterologous in length. For example, the H1 loop may contain anywhere between 2-8 residues compared to the usual five in Ig molecules. However, it is the CDR3 loop which exhibits greatest variation: in 17 camel antibody sequences reported, the length of this region varies between 7 and 21 residues (Muyldermans et al. 1994 Protein Eng 7: 1129-1135). Thirdly, many camelid VHH domains possess a disulphide linkage interconnecting CDR1 and CDR3 in the case of camels and interconnecting CDR1 and CDR2 in the case of llamas (Vu et al. 1997 Molec. Immunol. 34: 1121-113). The function of this structural feature appears to be maintenance of loop stability and providing a more contoured, as distinct from planar, loop conformation which both allows binding to pockets within the antigen and gives an increased surface area. However, not all camelid antibodies possess this disulphide bond, indicating that it is not an absolute structural requirement.

The present invention also relates to a method for generating and selecting single VLD molecules with novel binding affinities for target molecules (e.g., human serum albumin). This method involves the application of well known molecular evolution techniques to CTLA-4-derived polypeptides. The method may involve the production of phage or ribosomal display libraries for screening large numbers of mutated CTLA-4-derived polypeptides.

Filamentous fd-bacteriophage genomes are engineered such that the phage display, on their surface, proteins such as the Ig-like proteins (scFv, Fabs) which are encoded by the DNA that is contained within the phage (Smith, 1985 Science 228: 1315-1317; Huse et al. 1989 Science 246: 1275-81; McCafferty et al., 1990 Nature 348: 552-4; Hoogenboom et al., 1991 Nucleic Acids Res. 19: 4133-4137). Protein molecules can be displayed on the surface of Fd bacteriophage, covalently coupled to phage coat proteins encoded by gene III, or less commonly gene VIII. Insertion of antibody genes into the gene III coat protein gives expression of 3-5 recombinant protein molecules per phage, situated at the ends. In contrast, insertion of antibody genes into gene VIII has the potential to display about 2000 copies of the recombinant protein per phage particle, however this is a multivalent system which could mask the affinity of a single displayed protein. Fd phagemid vectors are also used, since they can be easily switched from the display of functional Ig-like fragments on the surface of fd-bacteriophage to secreting soluble Ig-like fragments in E. coli. Phage-displayed recombinant protein fusions with the N-terminus of the gene III coat protein are made possible by an amber codon strategically positioned between the two protein genes. In amber suppressor strains of E. coli, the resulting Ig domain-gene III fusions become anchored in the phage coat.

A selection process based on protein affinity can be applied to any high-affinity binding reagents such as antibodies, antigens, receptors and ligands (see, e.g., Winter and Milstein, 1991 Nature 349: 293-299, the entire contents of which are incorporated herein by reference). Thus, the selection of the highest affinity binding protein displayed on bacteriophage is coupled to the recovery of the gene encoding that protein. Ig- or non-Ig scaffold-displaying phage can be affinity selected by binding to cognate binding partners covalently coupled to beads or adsorbed to plastic surfaces in a manner similar to ELISA or solid phase radioimmunoassays. While almost any plastic surface will adsorb protein antigens, some commercial products are especially formulated for this purpose, such as Nunc Immunotubes.

Ribosomal display libraries involve polypeptides synthesized de novo in cell-free translation systems and displayed on the surface of ribosomes for selection purposes (Hanes and Pluckthun, 1997 Proc. Natl. Acad. Sci. USA. 94: 4937- 4942; He and Taussig, 1997 Nucl. Acids Res. 25: 5132-5134). The “cell-free translation system” comprises ribosomes, soluble enzymes required for protein synthesis (usually from the same cell as the ribosomes), transfer RNAs, adenosine triphosphate, guanosine triphosphate, a ribonucleoside triphosphate regenerating system (such as phosphoenol pyruvate and pyruvate kinase), and the salts and buffer required to synthesize a protein encoded by an exogenous mRNA. The translation of polypeptides can be made to occur under conditions which maintain intact polysomes, i.e. where ribosomes, mRNA molecule and translated polypeptides are associated in a single complex. This effectively leads to “ribosome display” of the translated polypeptide. For selection, the translated polypeptides, in association with the corresponding ribosome complex, are mixed with a target (e.g., serum albumin) molecule which is bound to a matrix (e.g., Dynabeads). The ribosomes displaying the translated polypeptides will bind the target molecule and these complexes can be selected and the mRNA re-amplified using RT-PCR.

Although there are several alternative approaches to modify binding molecules, the general approach for all displayed proteins conforms to a pattern in which individual binding reagents are selected from display libraries by affinity to their cognate ligand and/or receptor. The genes encoding these reagents are modified by any one or combination of a number of in vivo and in vitro mutation strategies and constructed as a new gene pool for display and selection of the highest affinity binding molecules.

Assessment of Binding Affinities

In certain embodiments, the dual-specific ligands of the present invention, including component molecules thereof (e.g., non-immunoglobulin molecules that bind human serum albumin) are assessed for binding affinity to target protein (e.g., human serum albumin). Binding of target protein epitopes can be measured by conventional antigen binding assays, such as ELISA, by fluorescence based techniques, including FRET, or by techniques such as surface plasmon resonance which measure the mass of molecules. Specific binding of an antigen-binding protein to an antigen or epitope can be determined by a suitable assay, including, for example, Scatchard analysis and/or competitive binding assays, such as radioimmunoassays (RIA), enzyme immunoassays such as ELISA and sandwich competition assays, and the different variants thereof.

Binding affinity is preferably determined using surface plasmon resonance (SPR) and the BIAcore (Karlsson et al., 1991), using a BIAcore system (Uppsala, Sweden). The BIAcore system uses surface plasmon resonance (SPR, Welford K. 1991, Opt. Quant. Elect. 23: 1; Morton and Myszka, 1998, Methods in Enzymology 295: 268) to monitor biomolecular interactions in real time, and uses surface plasmon resonance which can detect changes in the resonance angle of light at the surface of a thin gold film on a glass support as a result of changes in the refractive index of the surface up to 300 nm away. BIAcore analysis conveniently generates association rate constants, dissociation rate constants, equilibrium dissociation constants, and affmity constants. Binding affinity is obtained by assessing the association and dissociation rate constants using a BIAcore™ surface plasmon resonance system (BIAcore, Inc.). A biosensor chip is activated for covalent coupling of the target according to the manufacturer's (BIAcore) instructions. The target is then diluted and injected over the chip to obtain a signal in response units

(RU) of immobilized material. Since the signal in RU is proportional to the mass of immobilized material, this represents a range of immobilized target densities on the matrix. Dissociation data are fit to a one-site model to obtain k_{off}±s.d. (standard deviation of measurements). Pseudo-first order rate constant (Kd's) are calculated for each association curve, and plotted as a function of protein concentration to obtain kₒₙ±s.e. (standard error of fit). Equilibrium dissociation constants for binding, Kd's, are calculated from SPR measurements as k_{off}/kₒₙ.

As described by Phizicky and Field in Microb. Rev. (1995) 59: 114-115, a suitable antigen, such as HSA, is immobilized on a dextran polymer, and a solution containing a ligand for HSA, such as a single variable domain, flows through a cell, contacting the immobilized HSA. The single variable domain retained by immobilized HSA alters the resonance angle of impinging light, resulting in a change in refractive index brought about by increased amounts of protein, i.e. the single variable domain, near the dextran polymer. Since all proteins have the same refractive index and since there is a linear correlation between resonance angle shift and protein concentration near the surface, changes in the protein concentration at the surface due to protein/protein binding can be measured, see Phizicky and Field, supra. To determine a binding constant, the increase in resonance units is measured as a function of time by passing a solution of single variable domain protein past the immobilized ligand (HSA) until the RU values stabilize, then the decrease in RU is measured as a function of time with buffer lacking the single variable domain. This procedure is repeated at several different concentrations of single variable domain protein. Detailed theoretical background and procedures are described by R. Karlsson, et. al. (991) J. Immunol Methods, 145, 229.

The instrument software produces an equilibrium dissociation constant (Kd) as described above. An equilibrium dissociation constant determined through the use of Surface plasmon resonance (SPR) is described in U.S. Pat. No. 5,573,957, as being based on a table of dR_{A}/dt and R_{A} values, where R in this example is the HSA/single variable domain complex as measured by the BIAcore in resonance units and where dR/dt is the rate of formation of HSA/single variable domain complexes, i.e. the derivative of the binding curve; plotting the graph dR_{A}/dt vs. R_{A} for several different concentrations of single variable domain, and subsequently plotting the slopes of these lines vs. the concentration of single variable domain, the slope of this second graph being the association rate constant (M⁻¹, s⁻¹). The Dissociation Rate Constant or the rate at which the HSA and the single variable domain release from each other can be determined utilizing the dissociation curve generated on the BIAcore. By plotting and determining the slope of the log of the drop in response vs. time curve, the dissociation rate constant can be measured. The Equilibrium dissociation constant Kd=Dissociation Rate Constant/association rate constant.

A ligand according to any aspect of the present invention, includes a ligand having or consisting of at least one single variable domain, in the form of a monomer single variable domain or in the form of multiple single variable domains, i.e. a multimer. The ligand can be modified to contain additional moieties, such as a fusion protein, or a conjugate. Such a multimeric ligand, e.g., in the form of a dual-specific ligand, and/or such a ligand comprising or consisting of a single variable domain, i.e. a dAb monomer useful in constructing such a multimeric ligand, may advantageously dissociate from their cognate target(s) with a Kd of 300 nM or less, 300 nM to 5 pM (i.e., 3×10⁻⁷ to 5×10⁻¹²M), preferably 50 nM to 20 pM, or 5 nM to 200 pM or 1 nM to 100 pM, 1×10⁻⁷ M or less, 1×10⁻⁸ M or less, 1×10⁻⁹ M or less, 1×10⁻¹⁰ M or less, 1×10⁻¹¹ M or less; and/or a K_{off} rate constant ranging from 5×10⁻¹ to 1×10⁻⁷ S⁻¹, preferably 1×10⁻⁶ to 1×10⁻⁸ S⁻¹, preferably 1×10⁻² to 1×10⁻⁶ S⁻¹, or 5×10⁻³ to 1×10⁻⁵ S⁻¹, or 5×10⁻¹ S⁻¹ or less, or 1×10⁻² S⁻¹ or less, or 1×10⁻³ S⁻¹ or less, or 1×10⁻⁴ S⁻¹ or less, or 1×10⁻⁵ S⁻¹ or less, or 1×10⁻⁶ S⁻¹ or less as determined, for example, by surface plasmon resonance. The Kd rate constant is defined as K_{off}/Kₒₙ. Preferably, a single variable domain will specifically bind a target antigen or epitope with an affinity of less than 500 nM, preferably less than 200 nM, and more preferably less than 10 nM, such as less than 500 pM

Lipocalins

### Example 39

### Generation of Dual-Specific Ligand Comprising a Serum Albumin-Binding Lipocalin Non-Immunoglobulin Scaffold via Selection of Serum Albumin Binding Moieties

The bilin-binding protein (BBP), a lipocalin derived from Pieris brassicae can be reshaped by combinatorial protein design such that it recognizes human serum albumin. To this end, native BBP is subjected to library selection and, optionally, affinity maturation in order to produce human serum albumin-binding BBP molecules for use in dual-specific ligands of the invention.

The capability of a native BBP to bind human serum albumin is initially ascertained via BIAcore assay, as described infra for CTLA-4-derived polypeptides. (One of skill in the art will recognize that binding affinity can be assessed using any appropriate method, including, e.g., precipitation of labeled human serum albumin, competitive BIAcore assay, etc.) Following detection of no or low binding affinity (e.g., Kd values in the μM range or higher) of BBP for human serum albumin, at least one of a number of strategies are employed to impart human serum albumin binding properties to BBP, including one or more of the following methods that contribute to binding affinity.

Human serum albumin binding of BBP and BBP-derived polypeptide(s) is achieved and optimized via mutagenic methods, optionally in combination with parallel and/or iterative selection methods as described below and/or as otherwise known in the art. BBP polypeptide domains are subjected to randomized and/or NNK mutagenesis, performed as described infra. Such mutagenesis is performed upon the entirety of the BBP (or BBP-derived) polypeptide and/or is performed upon specific sequences within the BBP polypeptide, including 16 amino acid residues identified to reside at the center of the native BBP binding site, which is formed by four loops on top of an eight-stranded beta-barrel (Beste et al. 1999 Proc. Natl Acad. Sci. USA 96: 1898-903). Optionally, such mutagenesis procedures are randomized in order to evolve new or improved human serum albumin-binding polypeptides; and multiple rounds of mutagenesis may be performed during the process of creating a BBP that optimally binds to human serum albumin. PCR is optionally used to perform such methods of mutagenesis, resulting in the generation of sequence diversity across targeted sequences within the BBP (or BBP-derived) polypeptides. (Such approaches are similar to those described infra for dAb library generation.) In addition to random methods of mutagenesis, directed mutagenesis of targeted amino acid residues is employed where structural information establishes specific amino acid residues of BBP (or BBP-derived) polypeptides to be critical to binding of human serum albumin.

BBP (or BBP-derived) polypeptides engineered as described above are subjected to parallel and/or iterative selection methods to identify those BBP polypeptides that are optimized for human serum albumin binding. For example, following production of a library of mutagenized BBP polypeptide sequences, said library of polypeptides is displayed on phage and subjected to multiple rounds of selection requiring serum albumin binding and/or proliferation, as is described infra for selection of serum albumin-binding dAbs from libraries of dAbs. Optionally, selection is performed against serum albumin immobilized on immunotubes or against biotinlyated serum albumin in solution. Optionally, binding affinity is determined using surface plasmon resonance (SPR) and the BIAcore (Karlsson et al., 1991), using a BIAcore system (Uppsala, Sweden), with fully optimized BBP-derived polypeptides ideally achieving human serum albumin binding affinity Kd values in the nM range or better.

Following identification of BBP polypeptides that bind human serum albumin, such polypeptides are then used to generate dual-specific ligand compositions by any of the methods described infra.

Lipocalin Scaffold Proteins

The lipocalins (Pervaiz and Brew, FASEB J. 1 (1987), 209-214) are a family of small, often monomeric secretory proteins that have been isolated from various organisms, and whose physiological role lies in the storage or in the transport of different ligands as well as in more complex biological functions (Flower, Biochem. J. 318 (1996), 1-14). The lipocalins exhibit relatively little mutual sequence similarity and their belonging to the same protein structural family was first elucidated by X-ray structure analysis (Sawyer et al., Nature 327 (1987), 659).

The first lipocalin of known spatial structure was the retinol-binding protein, Rbp, which effects the transport of water-insoluble vitamin A in blood serum (Newcomer et al., EMBO J. 3 (1984), 1451-1454). Shortly thereafter, the tertiary structure of the bilin-binding protein, Bbp, from the butterfly Pieris brassicae was determined (Huber et al., J. Mol. Biol. 195 (1987), 423-434). The essential structural features of this class of proteins is illustrated in the spatial structure of this lipocalin. The central element in the folding architecture of the lipocalins is a cylindrical β-pleated sheet structure, a so-called β-barrel, which is made up of eight nearly circularly arranged antiparallel β-strands.

This supersecondary structural element can also be viewed as a “sandwich”-arrangement of two four-stranded β-sheet structures. Additional structural elements are an extended segment at the amino-terminus of the polypeptide chain and an α-helix close to the carboxy-terminus, which itself is followed by an extended segment. These additional features are, however, not necessarily revealed in all lipocalins. For example, a significant part of the N-terminal segment is missing in the epididymal retinoic acid-binding protein (Newcomer, Structure (1993) 1: 7-18). Additional peculiar structural elements are also known, such as, for example, membrane anchors (Bishop and Weiner, Trends Biochem. Sci. (1996) 21: 127) which are only present in certain lipocalins.

The β-barrel is closed on one end by dense amino acid packing as well as by loop segments. On the other end, the β-barrel forms a binding pocket in which the respective ligand of the lipocalin is complexed. The eight neighboring antiparallel β-strands there are connected in a respective pairwise fashion by hairpin bends in the polypeptide chain which, together with the adjacent amino acids which are still partially located in the region of the cylindrical β-pleated sheet structure, each form a loop element. The binding pocket for the ligands is formed by these in total four peptide loops. In the case of Bbp, biliverdin IXγ is complexed in this binding pocket. Another typical ligand for lipocalins is vitamin A in the case of Rbp as well as β-lactoglobulin (Papiz et al., Nature 324 (1986), 383-385).

As described, for example, in U.S. Publication No. 20060058510, members of the lipocalin family of polypeptides can be used to produce a class of molecules termed “anticalins” designed to recognize novel ligands via mutation of amino acids which are located in the region of the four peptide loops at the end of the cylindrical β-pleated sheet structure, and which are characterized in that they bind given ligands (e.g., human serum albumin) with a determinable affinity.

Ligand-binding sites of the lipocalins are constructed more simply than those of immunoglobulins. Lipocalin polypeptides comprise only one ring of 8 antiparallel β-strands: the β-barrel. This cyclic β-pleated sheet structure is conserved in the protein fold of the lipocalins. The binding site is formed in the entry region of the β-barrel by the four peptide loops, each of which connects two neighboring β-strands with one another. These peptide loops can vary significantly in their structure between the individual members of the lipocalin family.

To use a lipocalin polypeptide as a non-immunoglobulin scaffold, one or more of the four peptide loops forming the ligand-binding site of a lipocalin is subjected to mutagenesis, followed by choosing, i.e. selecting those protein variants (muteins), that exhibit the desired binding activity for a given ligand. The lipocalin muteins obtained in this way have been termed “anticalins”.

The four peptide loops of the lipocalins which, during production of anticalins, are modified in their sequence by mutagenesis, are characterized by those segments in the linear polypeptide sequence of BBP comprising amino acid positions 28 to 45, 58 to 69, 86 to 99 and 114 to 129 of Bbp. Each of these sequence segments begins before the C-terminus of one of the conserved β-strands at the open side of the β-barrel, includes the actual peptide hairpin, and ends after the N-terminus of the likewise conserved β-strand which follows in the sequence.

Sequence alignments or structural superpositions allow the sequence positions given for Bbp to be assigned to other lipocalins. For example, sequence alignments corresponding to the published alignment of Peitsch and Boguski (New Biologist 2 (1990), 197-206) reveal that the four peptide loops of ApoD include the amino acid positions 28 to 44, 59 to 70, 85 to 98 and 113 to 127. It is also possible to identify the corresponding peptide loops in new lipocalins which are suitable for mutagenesis in the same way.

In some cases, relatively weak sequence homology of the lipocalins may prove to be problematic in the determination of the conserved β-strands. It is therefore crucial that the polypeptide sequence be capable of forming the cyclic β-pleated sheet structure made of 8 antiparallel β-strands. This can be determined by employing methods of structural analysis such as protein crystallography or multidimensional nuclear magnetic resonance spectroscopy.

In non-Bbp lipocalins, such as, for example, ApoD or Rbp, sequence segments suitable for mutagenesis can easily be longer or shorter than that of Bbp based on the individually varying structure of the peptide loops. It can even be advantageous to additionally modify the length of sequence segments by deletion or insertion of one or more amino acids. In certain embodiments, those amino acid positions corresponding to sequence positions 34 to 37, 58, 60, 69, 88, 90, 93, 95, 97, 114, 116, 125, and 127 of Bbp are mutated. Correspondingly, in the case of ApoD, the sequence positions 34 to 37, 59, 61, 70, 87, 89, 92, 94, 96, 113, 115, 123 and 125 are preferred for mutagenesis. However, for the production of anticalins, not all of the sequence positions listed above have to be subjected to mutagenesis.

Other lipocalins are also suitable as an underlying structure for the production of anticalins. Preferably, the lipocalins Rbp, Bbp or ApoD, which presently have already been exhaustively studied biochemically, are used. The use of lipocalins of human origin is especially preferred for the production of anticalins. This especially applies when an application of the resulting anticalin(s) is intended for humans since, for example, in diagnostic or therapeutic applications in vivo, a minimal immunogenic effect is to be expected as compared to lipocalins from other organisms. However, other lipocalins as well as lipocalins which, possibly, have yet to be discovered can prove to be especially advantageous for the production of anticalins. Artificial proteins with a folding element which is structurally equivalent to the β-barrel of the lipocalins can also be used.

Preferably the anticalin molecules of the invention should be able to bind the desired ligand (e.g., human serum albumin) with a determinable affinity, i.e., with an affinity constant of at least 10⁵ M⁻¹. Affinities lower than this are generally no longer exactly measurable with common methods and are therefore of secondary importance for practical applications. Especially preferred are anticalins which bind the desired ligand with an affinity of at least 10⁶ M⁻¹, corresponding to a dissociation constant for the complex of 1 μM. The binding affinity of an anticalin to the desired ligand can be measured by the person skilled in the art by a multitude of methods, for example by fluorescence titration, by competition ELISA or by the technique of surface plasmon resonance.

The lipocalin cDNA, which can be produced and cloned by the person skilled in the art by known methods, can serve as a starting point for mutagenesis of the peptide loop, as it was for example described for Bbp (Schmidt and Skerra, Eur. J. Biochem. 219 (1994), 855-863). Alternatively, genomic DNA can also be employed for gene synthesis or a combination of these methods can be performed. For the mutagenesis of the amino acids in the four peptide loops, the person skilled in the art has at his disposal the various known methods for site-directed mutagenesis or for mutagenesis by means of the polymerase chain reaction. The mutagenesis method can, for example, be characterized in that mixtures of synthetic oligodeoxynucleotides, which bear a degenerate base composition at the desired positions, can be used for introduction of the mutations. The implementation of nucleotide building blocks with reduced base pair specificity, as for example inosine, is also an option for the introduction of mutations into the chosen sequence segment or amino acid positions. The procedure for mutagenesis of ligand-binding sites is simplified as compared to antibodies, since for the lipocalins only four instead of six sequence segments—corresponding to the four above cited peptide loops—have to be manipulated for this purpose.

In the methods of site-directed random mutagenesis implementing synthetic oligodeoxynucleotides, the relevant amino acid positions in the lipocalin structure which are to be mutated can be determined in advance. The ideal selection of the amino acid positions to be mutated can depend on the one hand on the lipocalin used, and on the other hand on the desired ligand (e.g., human serum albumin). It can be useful to maintain the total number of mutated amino acid positions within a single experiment low enough such that the collection of variants obtained by mutagenesis, i.e. the so-called library, can in its totality or, at least in a representative selection therefrom, be realized as completely as possible in its combinatorial complexity, not only at the level of the coding nucleic acids, but also at the level of the gene products.

It is possible to choose the amino acid positions to be mutated in a meaningful way especially when structural information exists pertaining to the lipocalin itself which is to be used, as is the case with BBP and Rbp or at least pertaining to a lipocalin with a similar structure, as for example in the case of ApoD. The set of amino acid positions chosen can further depend on the characteristics of the desired ligand. It can also prove advantageous to exclude single amino acid positions in the region of the ligand-binding pocket from mutagenesis if these, for example, prove to be essential for the folding efficiency or the folding stability of the protein. Specific oligonucleotide-based methods of lipocalin mutagenesis are described, for example, in U.S. Publication No. 20060058510, the entire contents of which are incorporated herein by reference.

After expressing the coding nucleic acid sequences subjected to mutagenesis, clones carrying the genetic information for anticalins which bind a given ligand (e.g., human serum albumin) can be selected from the differing clones of the library obtained. Known expression strategies and selection strategies can be implemented for the selection of these clones. Methods of this sort have been described in the context of the production or the engineering of recombinant antibody fragments, such as the “phage display” technique or “colony screening” methods (Skerra et al., Anal. Biochem. 196 (1991), 151-155).

Descriptions of “phage display” techniques are found, for example, in Hoess, Curr. Opin. Struct. Biol. 3 (1993), 572-579; Wells and Lowman, Curr. Opin. Struct. Biol. 2 (1992), 597-604; and Kay et al., Phage Display of Peptides and Proteins—A Laboratory Manual (1996), Academic Press. Briefly, in an exemplary embodiment, phasmids are produced which effect the expression of the mutated lipocalin structural gene as a fusion protein with a signal sequence at the N-terminus, preferably the OmpA-signal sequence, and with the coat protein pIII of the phage M13 (Model and Russel, in “The Bacteriophages”, Vol. 2 (1988), Plenum Press, New York, 375-456) or fragments of this coat protein, which are incorporated into the phage coat, at the C-terminus. The C-terminal fragment ApIII of the phage coat protein, which contains only amino acids 217 to 406 of the natural coat protein pIII, is preferably used to produce the fusion proteins. Especially preferred is a C-terminal fragment from pIII in which the cysteine residue at position 201 is missing or is replaced by another amino acid. Further description of phage display methods, selection methods, etc., that can be applied to lipocalins in production of “anticalins” possessing specific binding properties is detailed in, for example, U.S. Publication No. 20060058510, the entire contents of which are incorporated herein by reference.

Anticalins can be identified and produced, for example, using the above-described methods, to possess high affinity for a given ligand (e.g., human serum albumin). Ligand binding constants of more than 10⁶ M⁻¹ can be achieved for anticalins, even in cases where a novel ligand bears no structural relationship whatsoever to biliverdin IXγ, the original ligand of Bbp (refer to U.S. Publication No. 20060058510). Such affinities for novel ligands attainable with the anticalins are comparable with the affinities which are known for antibodies from the secondary immune response. Furthermore, there additionally exists the possibility to subject the anticalins produced to a further, optionally partial random mutagenesis in order to select variants of even higher affinity from the new library thus obtained. Corresponding procedures have already been described for the case of recombinant antibody fragments for the purpose of an “affinity maturation” (Low et al., J. Mol. Biol. 260 (1996), 359-368; Barbas and Burton, Trends Biotechnol. 14 (1996), 230-234) and can also be applied to anticalins in a corresponding manner by the person skilled in the art.

Staphylococcal Protein A (SPA)/Affibody

### Example 40

### Generation of Dual-Specific Ligand Comprising a Serum Albumin-Binding Affibody (Staphylococcal protein A (SPA)) Non-Immunoglobulin Scaffold via Selection of Serum Albumin Binding Moieties

The Z domain of staphylococcal protein A (SPA) is subjected to library selection and, optionally, affinity maturation techniques in order to produce human serum albumin-binding SPA-derived non-immunoglobulin scaffold molecules (termed “affibodies”) for use in dual-specific ligands of the invention.

Real-time binding analysis by BIAcore is performed to assess whether human serum albumin specifically binds to immobilized SPA polypeptide. (One of skill in the art will recognize that binding affinity can be assessed using any appropriate method, including, e.g., precipitation of labeled human serum albumin, competitive BIAcore assay, etc.) Following detection of no or low binding affinity. (e.g., Kd values in the μM range or higher) of an unaltered SPA polypeptide for human serum albumin, at least one of a number of strategies are employed to impart human serum albumin binding properties to the SPA polypeptide, including one or more of the following methods designed to impart and/or enhance binding affinity of the molecule for target antigen.

Human serum albumin binding of SPA scaffold polypeptide(s) is achieved and optimized via mutagenic methods, optionally in combination with parallel and/or iterative selection methods as described below and/or as otherwise known in the art. SPA scaffold polypeptide domains are subjected to randomized and/or NNK mutagenesis, performed as described infra. Such mutagenesis is performed upon the entirety of the Z domain of the SPA polypeptide or upon specific sequences within the SPA polypeptide, e.g., upon 13 solvent-accessible surface residues of domain Z as identified in Nord et al. (1997 Nat. Biotechnol. 15: 772-77), and is optionally randomized in order to evolve new or improved human serum albumin-binding polypeptides. PCR is optionally used to perform such methods of mutagenesis, resulting in the generation of sequence diversity across targeted sequences within the SPA polypeptides. (Such approaches are similar to those described infra for dAb library generation.) In addition to random methods of mutagenesis, directed mutagenesis of targeted amino acid residues is employed where structural information establishes specific amino acid residues of SPA polypeptides to be critical to binding of human serum albumin. In certain embodiments, repertoires of mutant Z domain genes are assembled and inserted into a phagemid vector adapted for monovalent phage display. Libraries comprising, e.g., millions of transformants, are constructed using, e.g., NN(G/T) or alternative (C/A/G)NN degeneracy for mutagenesis.

SPA polypeptides engineered as described above are subjected to parallel and/or iterative selection methods to identify those SPA polypeptides that are optimized for human serum albumin binding. For example, following production of a library of mutagenized SPA polypeptide sequences, said library of polypeptides is displayed on phage and subjected to multiple rounds of selection requiring serum albumin binding and/or proliferation, as is described infra for selection of serum albumin-binding dAbs from libraries of dAbs. Biopanning against the human serum albumin target protein is performed to achieve significant enrichment for serum albumin binding SPA molecules. Selected clones are subsequently expressed in E. coli and analyzed by SDS-PAGE, circular dichroism spectroscopy, and binding studies to human serum albumin by biospecific interaction analysis. The SPA molecules (affibodies) that bind to human serum albumin are anticipated to have a secondary structure similar to the native Z domain and have micromolar dissociation constants (Kd) for their respective targets in the range of μM or better (e.g., nM or pM).

Optionally, selection is performed against serum albumin immobilized on immunotubes or against biotinlyated serum albumin in solution. Optionally, binding affinity is determined using surface plasmon resonance (SPR) and the BIAcore (Karlsson et al., 1991), using a BIAcore system (Uppsala, Sweden), with fully optimized SPA-derived polypeptides ideally achieving human serum albumin binding affinity Kd values in the nM range or better.

Following identification of SPA polypeptides that bind human serum albumin, such polypeptides are then used to generate dual-specific ligand compositions by any of the methods described infra.

Staphylococcal Protein A (SPA) Affibody Polypeptides

Solvent-exposed surfaces of bacterial receptors can be targeted for random mutagenesis followed by phenotypic selection for purpose of imparting, e.g., binding affinity for serum albumin to such receptor molecules. Such proteins can be unusually stable, which makes them suitable for various applications (Alexander et al. (1992) Biochemistry 31: 3597-3603). In particular, for bacterial receptors containing helix bundle structures, the conformation can be expected to be tolerant to changes in the side chains of residues not involved in helix packing interfaces. Examples of such molecules are the relatively small (58 residues) IgG-binding domain B of staphylococcal protein A (SPA) and the synthetic analogue of domain B, designated domain Z (Nilsson et al. (1987) Protein Engineering 1: 107-113).

The SPA-derived domain Z is the primary domain of SPA utilized as a scaffold for purpose of constructing domain variants with novel binding properties (refer to, e.g., WO 00/63243 and WO 95/19374, incorporated herein by reference in their entireties). The SPA Z domain is a 58 amino acid residue cysteine-free three-helix bundle domain that is used as a scaffold for construction of combinatorial phagemid libraries from which variants are selected that target desired molecules (e.g., human serum albumin) using phage display technology (Nilsson et al. 1987 Protein Eng. 1: 107-113; Nord et al. 1997 Nat. Biotechnol. 15: 772-777; Nord et al. 2000 J. Biotechnol. 80: 45-54; Hansson et al. 1999 Immunotechnology 4: 237-252; Eklund et al., 2002 Proteins 48: 454-462; Rönnmark et al. 2002 Eur. J. Biochem. 269: 2647-2655). Such target-binding variants, termed “affibody” molecules, are selected as binders to target proteins by phage display of combinatorial libraries in which typically 13 side-chains on the surface of helices 1 and 2 (Q9, Q10, N11, F13, Y14, L17, H18, E24, E25, R27, N28, Q32 and K35) in the Z domain have been randomized (Lendel et al. 2006 J. Mol. Biol. 359: 1293-304). The simple, robust structure of such affibody molecules, together with their low molecular weight (7 Kda), make them suitable for a wide variety of applications. Documented efficacy has been shown in bioprocess- and laboratory-scale bioseparations (Nord et al. 2000 J. Biotechnol. 80: 45-54; Nord et al. 2001 Eur. J. Biochem. 268: 4269-4277; Gräslund et al. 2002 J. Biotechnol. 99: 41-50), and promising results have been obtained when evaluating affibody ligands as detection reagents (Karlström and Nygren 2001 Anal. Biochem. 295: 22-30; Rönnmark et al. 2002 J. Immunol. Methods 261: 199-211), to engineer adenoviral tropism (Henning et al. 2002 Hum. Gene Ther. 13: 1427-1439) and to inhibit receptor interactions (Sandström et al. 2003 Protein Eng. 16: 691-697). Thus, engineered affibody ligands that, e.g., bind to human serum albumin are desirable components of certain dual-specific ligand compositions of the present invention.

Libraries of polypeptides derived from the Z domain of staphylococcal protein A may be generated by any method of mutagenesis as known in the art and/or as described infra. Following creation of such polypeptide libraries, variants capable of binding desired target molecules (e.g., human serum albumin) can be efficiently selected and identified using, for example, in vitro selection technologies such as phage display (Dunn 1996; Smith and Patrenko 1997; Hoogenboom et al. 1998), ribosomal display (Hanes and Pluckthun 1997; He and Taussig 1997) peptides on plasmids (Schatz 1993) or bacterial display (Georgiou et al. 1997). For such selections, a correlation between library size (complexity) and the likelihood of isolating binders of higher affinities (KD=10⁻⁸ M or lower) has been theoretically considered (Perelson and Oster 1979) and experimentally demonstrated (Griffiths et al. 1994; Vaughan et al. 1996; Aujame et al. 1997).

Affibodies have several advantages over traditional antibodies, e.g. (i) a lower cost of manufacture; (ii) smaller size; (iii) increased stability and robustness; and (iv) the ability of being produced recombinantly in a bacterial host, or by chemical synthesis, which obviates the risk for viral contamination.

An affibody is a polypeptide which is a derivative of a staphylococcal protein A (SPA) domain, said SPA domain being the B or Z domain, wherein a number of the amino acid residues have been substituted by other amino acid residues, said substitution being made without substantial loss of the basic structure and stability of the said SPA domain, and said substitution resulting in interaction capacity of the said polypeptide with at least one domain of a target antigen (e.g., human serum albumin). The number of substituted amino acid residues could be from 1 to about 30, or from 1 to about 13. Other possible ranges are from 4 to about 30; from 4 to about 13; from 5 to about 20, or from 5 to about 13 amino acid residues. It will be understood by the skilled person, e.g., from Nord et al. 1997 Nat. Biotechnol. 15: 772-777, that preferentially amino residues located on the surface of the Z-domain can be substituted, while the core of the bundle should be kept constant to conserve the structural properties of the molecule.

A process for the manufacture of an affibody is set forth, e.g., in WO 00/63243, and for purposes of the present invention could involve, e.g., the following steps: (i) displaying, by e.g. phage display (for a review, see, e.g., Kay, K. et al. (eds.) Phage Display of Peptides and Proteins: A Laboratory Manual, Academic Press, San Diego, ISBN 0-12-4023 80-0), ribosomal display (for a review, see e.g. Hanes, J. et al. (1998) Proc. Natl. Acad. Sci. USA 95: 14130-14135) or cell display (for a review, see e.g. Daugherty, P. S. et al. (1998) Protein Eng. 11: 825-832), polypeptide variants from a protein library embodying a repertoire of polypeptide variants derived from SPA domain B or Z; (ii) selecting clones expressing polypeptides that bind to human serum albumin; and (iii) producing polypeptides by recombinant expression of the selected clones or by chemical synthesis.

Avimer

### Example 41

### Generation of Dual-Specific Ligand Comprising a Serum Albumin-Binding Avimer via CDR Grafting

The CDR domains of dAb7h14 are used to construct an avimer polypeptide that binds human serum albumin in the following manner. The CDR1 (RASQWIGSQLS; SEQ ID NO.:______) CDR2 (WRSSLQS; SEQ ID NO.:______), and CDR3 (AQGAALPRT; SEQ ID NO.:______) sequences of dAb7h14 are grafted into a C2 monomer (described in US Patent Publication No. 2005/0221384, incorporated herein by reference in its entirety) at residues 17-28, 49-53 and 78-85, respectively, which constitute the loop regions 1, 2 and 3, respectively of the C2 monomer. Real-time binding analysis by BIAcore is performed to assess whether human serum albumin specifically binds to immobilized C2-derived monomer polypeptide comprising the anti-human serum albumin CDR domains of dAb7h14. (One of skill in the art will recognize that binding affinity can be assessed using any appropriate method, including, e.g., precipitation of labeled human serum albumin, competitive BIAcore assay, etc.) If no or low human serum albumin affinity (e.g., Kd values in the μM range or higher) is detected, at least one of a number of strategies are employed to improve the human serum albumin binding properties of the CDR-grafted C2 monomer (and/or of avimer dimers, trimers and other higher-order iteration compositions), including any of the following methods that contribute to binding affinity.

The length(s) of dAb7h14 CDR-grafted regions of the initial C2 monomer polypeptide (and/or of iteratively-produced avimer dimer, trimer, etc. polypeptides) corresponding to solvent-exposed loop regions within the native C2 monomer (and/or within other native monomers used in the avimer compositions) are adjusted through the use of linker polypeptides. For example, the nine amino acid residue CDR3 peptide sequence of dAb7h14 can be extended to 13 amino acid residues in length using amino acid linkers of, e.g., zero to four residues in length located on either and/or both the N- or C-terminal flanks of the dAb7h14 CDR3 polypeptide sequence, thereby achieving a total grafted peptide sequence length of 13 amino acids within the CDR3-grafted domain corresponding to loop 3 of the C2 monomer polypeptide. Such use of linker polypeptide(s) is optionally combined with mutagenesis of the linker sequences, CDR sequences and/or non-CDR C2 monomer polypeptide sequences (e.g., using mutagenic optimization procedures as described below), in order to improve the human serum albumin binding capability of CDR-grafted C2 monomer polypeptide(s) (e.g., via optimization of both CDR and C2 monomer polypeptide sequences within the CDR-grafted C2 monomer polypeptides). The polypeptide linkers employed for such purpose either possess a predetermined sequence, or, optionally, are selected from a population of randomized polypeptide linker sequences via assessment of the human serum albumin binding capabilities of linker-containing CDR-grafted C2 monomer polypeptides. Optimization methods are performed in parallel and/or iteratively. Both parallel and iterative optimization (e.g., affinity maturation) processes employ selection methods as described below and/or as known in the art as useful for optimization of polypeptide binding properties.

Human serum albumin binding of CDR-grafted C2 monomer polypeptide(s) (and/or of avimer dimer, trimer, etc. iteratively-produced higher-order compositions, or individual additional monomers contributing to same) presenting dAb7h14 CDRs is optimized via mutagenesis, optionally in combination with parallel and/or iterative selection methods as described below and/or as otherwise known in the art. For the exemplary C2 monomer scaffold polypeptide, domains surrounding grafted dAb7h14 CDR polypeptide sequences are subjected to randomized and/or NNK mutagenesis, performed as described infra. Such mutagenesis is optionally performed within the C2 monomer polypeptide sequence upon selected amino acid residues as set forth, e.g., in US Patent Publication No. 2005/0221384, or is optionally performed upon all non-CDR amino acid residues, and is optionally randomized in order to evolve new or improved human serum albumin-binding polypeptides. Optionally, dAb7h14 CDR polypeptide domains presented within the CDR-grafted C2 monomer polypeptide are subjected to mutagenesis via, e.g., random mutagenesis, NNK mutagenesis, look-through mutagenesis and/or other art-recognized method. PCR is optionally used to perform such methods of mutagenesis, resulting in the generation of sequence diversity across targeted sequences within the CDR-grafted C2 monomer polypeptides. Such approaches are similar to those described infra for dAb library generation. In addition to random and/or look-through methods of mutagenesis, directed mutagenesis of targeted amino acid residues is employed where structural information establishes specific amino acid residues to be critical to binding of human serum albumin.

C2 monomer polypeptides (and/or iteratively produced avimer compositions comprising individual monomers) comprising grafted dAb7h14 CDR sequences engineered as described above are subjected to parallel and/or iterative selection methods to identify those C2 monomer polypeptides (and avimer compositions) that are optimized for human serum albumin binding. For example, following production of a library of dAb7h14 CDR-grafted C2 monomer polypeptide sequences, this library of such polypeptides is displayed on phage and subjected to multiple rounds of selection requiring serum albumin binding and/or proliferation, as is described infra for selection of serum albumin-binding dAbs from libraries of dAbs. Optionally, selection is performed against serum albumin immobilized on immunotubes or against biotinlyated serum albumin in solution. Optionally, binding affinity is determined using surface plasmon resonance (SPR) and the BIAcore (Karlsson et al., 1991), using a BIAcore system (Uppsala, Sweden), with fully optimized avimers comprising C2-derived monomers ideally achieving human serum albumin binding affinity Kd values in the nM range or better.

Upon identification of C2 monomer-derived polypeptides that bind human serum albumin, human serum binding properties of such initial monomers may be further enhanced via combination of such monomers with other monomers, followed by further mutagenesis and/or selection, thereby forming an avimer composition possessing specific affinity for human serum albumin. Following identification of an avimer composition possessing affinity for human serum albumin, such avimer polypeptides are then used to generate dual-specific ligand compositions by any of the methods described infra.

### Example 42

### Generation of Dual-Specific Ligand Comprising a Serum Albumin-Binding Avimer Non-Immunoglobulin Scaffold via Selection of Serum Albumin Binding Moieties

The native C2 monomer polypeptide as set forth in is subjected to library selection and, optionally, affinity maturation techniques, then combined with an additional monomer (e.g., a fibronectin monomer, for which human serum albumin affinity optionally can be optimized in parallel) and optionally iteratively subjected to library selection and, optionally, affinity maturation techniques in order to produce a human serum albumin-binding avimer non-immunoglobulin scaffold molecule for use in dual-specific ligands of the invention.

Real-time binding analysis by BIAcore is performed to assess whether human serum albumin specifically binds to an immobilized C2 monomer polypeptide (and/or an iteratively-produced avimer molecule). Following detection of no or low binding affinity (e.g., Kd values in the μM range or higher) of a C2 monomer polypeptide for human serum albumin, at least one of a number of strategies are employed to impart human serum albumin binding properties to the C2 monomer polypeptide, including one or more of the following methods that contribute to binding affinity.

Human serum albumin binding of C2 monomer polypeptide(s) (and/or iteratively produced avimer dimer, trimer, etc. molecules) is achieved and optimized via mutagenic methods, optionally in combination with parallel and/or iterative selection methods as described below and/or as otherwise known in the art. C2 monomer polypeptide domains are subjected to randomized and/or NNK mutagenesis, performed as described infra. Such mutagenesis is performed upon the entirety of the C2 monomer polypeptide or upon specific sequences within the C2 monomer polypeptide upon selected amino acid residues as set forth, e.g., in US Patent Publication No. 2005/0221384, and is optionally randomized in order to evolve new or improved human serum albumin-binding polypeptides. PCR is optionally used to perform such methods of mutagenesis, resulting in the generation of sequence diversity across targeted sequences within the C2 monomer polypeptides and/or avimer molecules. (Such approaches are similar to those described infra for dAb library generation.) In addition to random methods of mutagenesis, directed mutagenesis of targeted amino acid residues is employed where structural information establishes specific amino acid residues of C2 monomer and/or avimer molecules to be critical to binding of human serum albumin.

C2 monomer polypeptides engineered as described above are subjected to parallel and/or iterative selection methods to identify those C2 monomer polypeptides and/or avimer molecules that are optimized for human serum albumin binding. For example, following production of a library of mutagenized C2 monomer polypeptide sequences, said library of polypeptides is displayed on phage and subjected to multiple rounds of selection requiring serum albumin binding and/or proliferation, as is described infra for selection of serum albumin-binding dAbs from libraries of dAbs. Optionally, the rounds of selection may include iterations within which additional monomer subunits are added to form a new avimer molecule. Optionally, selection is performed against serum albumin immobilized on immunotubes or against biotinlyated serum albumin in solution. Optionally, binding affinity is determined using surface plasmon resonance (SPR) and the BIAcore (Karlsson et al., 1991), using a BIAcore system (Uppsala, Sweden), with fully optimized avimers comprising C2-derived monomer polypeptides ideally achieving human serum albumin binding affinity Kd values in the nM range or better.

Upon identification of C2 monomer-derived polypeptides that bind human serum albumin, human serum binding properties of such initial monomers may be further enhanced via combination of such monomers with other monomers, followed by further mutagenesis and/or selection, thereby forming an avimer composition possessing specific affinity for human serum albumin. Following identification of an avimer composition possessing affinity for human serum albumin, such avimer polypeptides are then used to generate dual-specific ligand compositions by any of the methods described infra.

Production and Use of Avimer Polypeptides

Avimers are evolved from a large family of human extracellular receptor domains by in vitro exon shuffling and phage display, generating multidomain proteins with binding and/or inhibitory properties. Linking multiple independent binding domains (selected, e.g., in iterative fashion for binding to a target protein, e.g., human serum albumin) creates avidity and results in improved affinity and specificity compared with conventional single-epitope binding proteins. Other potential advantages include simple and efficient production of multitarget-specific molecules in E. coli, improved thermostability and resistance to proteases. Avimers can be produced that possess sub-nM affinities against a target protein. For example, an avimer that inhibits interleukin 6 with 0.8 pM IC₅₀ in cell-based assays has been produced and characterized as biologically active (Silverman et al. 2005 Nature Biotechnology 23: 1556-1561; also see, for example, U.S. Patent Application Publ. Nos. 2005/0221384, 2005/0164301, 2005/0053973 and 2005/0089932, 2005/0048512, and 2004/0175756, each of which is hereby incorporated by reference herein in its entirety).

Avimer synthesis involves phage display libraries derived from the human repertoire of A domains. Synthetic recombination is used to create a highly diverse pool of monomers, as described in Silverman et al. (2005 Nature Biotechnology 23: 1556-1561). Following generation of a pool of monomers, the pool is screened against target protein (e.g., human serum albumin). Initial candidates are identified, and an additional monomer is added and the resulting dimer library is screened against the target protein to identify candidate target-binding dimers. The method is then iterated to obtain a trimer with very high binding affmity for the target protein, and, optionally, may be iterated further to identify higher order candidate complexes. Candidate complexes that are identified to bind with high affinity and specificity to target proteins are termed avimers (for “avidity multimer”).

Monomer domains of avimers can be polypeptide chains of any size. For example, monomer domains can have about 25 to about 500, about 30 to about 200, about 30 to about 100, about 90 to about 200, about 30 to about 250, about 30 to about 60, about 9 to about 150, about 100 to about 150, about 25 to about 50, or about 30 to about 150 amino acids. Similarly, a monomer domain of an avimer can comprise, e.g., from about 30 to about 200 amino acids; from about 25 to about 180 amino acids; from about 40 to about 150 amino acids; from about 50 to about 130 amino acids; or from about 75 to about 125 amino acids. Monomer domains and immuno-domains can typically maintain stable conformation in solution. Sometimes, monomer domains of avimers and immuno-domains can fold independently into a stable conformation. The stable conformation can be stabilized by metal ions. The stable conformation can optionally contain disulfide bonds (e.g., at least one, two, or three or more disulfide bonds). The disulfide bonds can optionally be formed between two cysteine residues.

Publications describing monomer domains and mosaic proteins and references cited within include the following: Hegyi, H and Bork, P. 1997 J. Protein Chem., 16: 545-551; Baron et al. 1991 Trends Biochem. Sci. 16: 13-17; Ponting et al. 2000 Adv. Protein Chem. 54: 185-244; Doolittle 1995 Annu. Rev. Biochem 64: 287-314; Doolitte and Bork 1993 Scientific American 269: 50-6; and Bork 1991 FEBS letters 286: 47-54. Monomer domains used in avimers can also include those domains found in Pfam database and the SMART database. See Schultz et al. 2000 Nucleic Acid Res. 28: 231-34.

Monomer domains that are particularly suitable for use in avimer compositions are (1) β-sandwich domains; (2) β-barrel domains; or (3) cysteine-rich domains comprising disulfide bonds. Cysteine-rich domains employed in avimers typically do not form an α-helix, a β-sheet, or a β-barrel structure. Typically, the disulfide bonds promote folding of the domain into a three-dimensional structure. Usually, cysteine-rich domains have at least two disulfide bands, more typically at least three disulfide bonds.

Monomer domains of avimers can have any number of characteristics. For example, the domains can have low or no immunogenicity in an animal (e.g., a human). Domains can have a small size, for example, the domains may be small enough to penetrate skin or other tissues. Domains can possess a range of in vivo half-lives or stabilities.

Illustrative monomer domains suitable for use in avimer compositions include, e.g., an EGF-like domain, a Kringle-domain, a fibronectin type I domain, a fibronectin type II domain, a fibronectin type III domain, a PAN domain, a Gla domain, a SRCR domain, a Kunitz/Bovine pancreatic trypsin Inhibitor domain, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an Immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, and other such domains known to those of ordinary skill in the art, as well as derivatives and/or variants thereof. US Patent Publication No. 20050221384 presents schematic diagrams of various exemplary forms of monomer domains found in molecules in the LDL-receptor family.

Suitable monomer domains (e.g., domains with the ability to fold independently or with some limited assistance) can be selected from the families of protein domains that contain β-sandwich or β-barrel three dimensional structures as defined by such computational sequence analysis tools as Simple Modular Architecture Research Tool (SMART; see Shultz et al. 2000 Nucleic Acids Research 28: 231-234) or CATH (see Pearl et al. 2000 Nucleic Acids Research 28: 277-282). Exemplary monomer domains of avimers also include domains of fibronectin type III domain, an anticalin domain and a Ig-like domain from CTLA-4. Some aspects of these domains are described in WO 01/64942 by Lipovsek et al., WO99/16873 by Beste et al., and WO 00/60070 by Desmet et al., the contents of which are incorporated in their entirety herein by reference.

Monomer domains of avimers are optionally cysteine rich. Suitable cysteine rich monomer domains include, e.g., the LDL receptor class A domain (“A-domain”) or the EGF-like domain. The monomer domains can also have a cluster of negatively charged residues. Optionally, the monomer domains contain a repeated sequence, such as YWTD as found in the β-Propeller domain. Another exemplary monomer domain suitable for use in avimers is the C2 domain. Exemplary A domain and C2 domain sequences and consensus sequences useful in avimer production, including exemplary selections of amino acid residues (e.g., surface-exposed loop residues) most desirable for mutagenic targeting, are presented in US Patent Publication No. 2005/0221384.

Polynucleotides (also referred to as nucleic acids) encoding the monomer domains are typically employed to make monomer domains via expression. Nucleic acids that encode monomer domains can be derived from a variety of different sources. Libraries of monomer domains can be prepared by expressing a plurality of different nucleic acids encoding naturally occurring monomer domains, altered monomer domains (i.e., monomer domain variants), or a combinations thereof.

Monomer domains that bind to a selected or desired ligand (e.g., human serum albumin) or mixture of ligands are identified, optionally as an initial step in avimer production. In some embodiments, monomer domains and/or immuno-domains are identified or selected for a desired property (e.g., binding affinity for human serum albumin) and then the monomer domains and/or immuno-domains are formed into multimers. For those embodiments, any method resulting in selection of domains with a desired property (e.g., human serum albumin binding) can be used. For example, the methods can comprise providing a plurality of different nucleic acids, each nucleic acid encoding a monomer domain; translating the plurality of different nucleic acids, thereby providing a plurality of different monomer domains; screening the plurality of different monomer domains for binding of the desired ligand or a mixture of ligands; and, identifying members of the plurality of different monomer domains that bind the desired ligand or mixture of ligands.

Monomer domains for avimer production can be naturally-occurring or altered (non-natural variants). The term “naturally occurring” is used herein to indicate that an object can be found in nature. For example, natural monomer domains can include human monomer domains or optionally, domains derived from different species or sources, e.g., mammals, primates, rodents, fish, birds, reptiles, plants, etc. The natural occurring monomer domains can be obtained by a number of methods, e.g., by PCR amplification of genomic DNA or cDNA. The term “native”, as used herein, is used in reference to a nucleic acid and/or polypeptide that has not been altered via mutagenesis or otherwise via performance of any of the methods described infra.

Monomer domains of avimers can be naturally-occurring domains or non-naturally occurring variants. Libraries of monomer domains employed in synthesis of avimers may contain naturally-occurring monomer domain, non-naturally occurring monomer domain variants, or a combination thereof.

A variety of reporting display vectors or systems can be used to express nucleic acids encoding monomer domains and avimers, and to test for a desired activity (e.g., human serum albumin binding). For example, a phage display system is a system in which monomer domains are expressed as fusion proteins on the phage surface (Pharmacia, Milwaukee Wis.). Phage display can involve the presentation of a polypeptide sequence encoding monomer domains and/or immuno-domains on the surface of a filamentous bacteriophage, typically as a fusion with a bacteriophage coat protein. Exemplary methods of affinity enrichment and phage display are set forth, for example, in PCT patent publication Nos. 91/17271, 91/18980, and 91/19818 and 93/08278, incorporated herein by reference in their entireties.

Examples of other display systems include ribosome displays, a nucleotide-linked display (see, e.g., U.S. Pat. Nos. 6,281,344; 6,194,550, 6,207,446, 6,214,553, and 6,258,558), cell surface displays and the like. The cell surface displays include a variety of cells, e.g., E. coli, yeast and/or mammalian cells. When a cell is used as a display, the nucleic acids, e.g., obtained by PCR amplification followed by digestion, are introduced into the cell and translated. Optionally, polypeptides encoding monomer domains or avimers can be introduced, e.g., by injection, into the cell.

As described infra and in the art, avimers are multimeric compositions. In exemplary embodiments, multimers comprise at least two monomer domains and/or immuno-domains. For example, multimers of the invention can comprise from 2 to about 10 monomer domains and/or immuno-domains, from 2 and about 8 monomer domains and/or immuno-domains, from about 3 and about 10 monomer domains and/or immuno-domains, about 7 monomer domains and/or immuno-domains, about 6 monomer domains and/or immuno-domains, about 5 monomer domains and/or immuno-domains, or about 4 monomer domains and/or immuno-domains. In some embodiments, the multimer comprises at least 3 monomer domains and/or immuno-domains. Typically, the monomer domains have been pre-selected for binding to the target molecule of interest (e.g., human serum albumin).

Within an avimer, each monomer domain may specifically bind to one target molecule (e.g., human serum albumin). Optionally, each monomer binds to a different position (analogous to an epitope) on a target molecule. Multiple monomer domains and/or immuno-domains that bind to the same target molecule can result in an avidity effect resulting in improved avidity of the multimer avimer for the target molecule compared to each individual monomer. Optionally, the multimer can possess an avidity of at least about 1.5, 2, 3, 4, 5, 10, 20, 50 or 100 times the avidity of a monomer domain alone for target protein (e.g., human serum albumin).

Selected monomer domains can be joined by a linker to form a multimer (avimer). For example, a linker is positioned between each separate discrete monomer domain in a multimer. Typically, immuno-domains are also linked to each other or to monomer domains via a linker moiety. Linker moieties that can be readily employed to link immuno-domain variants together are the same as those described for multimers of monomer domain variants. Exemplary linker moieties suitable for joining immuno-domain variants to other domains into multimers are described herein.

Joining of selected monomer domains via a linker to form an avimer can be accomplished using a variety of techniques known in the art. For example, combinatorial assembly of polynucleotides encoding selected monomer domains can be achieved by DNA ligation, or optionally, by PCR-based, self-priming overlap reactions. The linker can be attached to a monomer before the monomer is identified for its ability to bind to a target multimer or after the monomer has been selected for the ability to bind to a target multimer.

As mentioned above, the polypeptide(s) comprising avimers can be altered. Descriptions of a variety of diversity generating procedures for generating modified or altered nucleic acid sequences encoding these polypeptides are described above and below in the following publications and the references cited therein: Soong, N. et al., Molecular breeding of viruses, (2000) Nat Genet 25(4):436-439; Stemmer, et al., Molecular breeding of viruses for targeting and other clinical properties, (1999) Tumor Targeting 4:1-4; Ness et al., DNA Shuffling of subgenomic sequences of subtilisin, (1999) Nature Biotechnology 17:893-896; Chang et al., Evolution of a cytokine using DNA family shuffling, (1999) Nature Biotechnology 17:793-797; Minshull and Stemmer, Protein evolution by molecular breeding, (1999) Current Opinion in Chemical Biology 3:284-290; Christians et al., Directed evolution of thymidine kinase for AZT phosphorylation using DNA family shuffling, (1999) Nature Biotechnology 17:259-264; Crameri et al.,

DNA shuffling of a family of genes from diverse species accelerates directed evolution, (1998) Nature 391:288-291; Crameri et al., Molecular evolution of an arsenate detoxification pathway by DNA shuffling, (1997) Nature Biotechnology 15:436-438; Zhang et al., Directed evolution of an effective fucosidase from a galactosidase by DNA shuffling and screening (1997) Proc. Natl. Acad. Sci. USA 94:4504-4509; Patten et al., Applications of DNA Shuffling to Pharmaceuticals and Vaccines, (1997) Current Opinion in Biotechnology 8:724-733; Crameri et al., Construction and evolution of antibody-phage libraries by DNA shuffling, (1996) Nature Medicine 2:100-103; Crameri et al., Improved green fluorescent protein by molecular evolution using DNA shuffling, (1996) Nature Biotechnology 14:315-319; Gates et al., Affinity selective isolation of ligands from peptide libraries through display on a lac repressor ‘headpiece dimer’, (1996) Journal of Molecular Biology 255:373-386; Stemmer, Sexual PCR and Assembly PCR, (1996) In: The Encyclopedia of Molecular Biology. VCH Publishers, New York. pp. 447-457; Crameri and Stemmer, Combinatorial multiple cassette mutagenesis creates all the permutations of mutant and wildtype cassettes, (1995) BioTechniques 18:194-195;

Stemmer et al., Single-step assembly of a gene and entire plasmid form large numbers of oligodeoxy-ribonucleotides, (1995) Gene, 164:49-53; Stemmer, The Evolution of Molecular Computation, (1995) Science 270:1510; Stemmer. Searching Sequence Space, (1995) Bio/Technology 13:549-553; Stemmer, Rapid evolution of a protein in vitro by DNA shuffling, (1994) Nature 370:389-391; and Stemmer, DNA shuffling by random fragmentation and reassembly: In vitro recombination for molecular evolution, (1994) Proc. Natl. Acad. Sci. USA 91:10747-10751.

Mutational methods of generating diversity include, for example, site-directed mutagenesis (Ling et al., Approaches to DNA mutagenesis: an overview, (1997) Anal Biochem. 254(2): 157-178; Dale et al., Oligonucleotide-directed random mutagenesis using the phosphorothioate method, (1996) Methods Mol. Biol. 57:369-374; Smith, In vitro mutagenesis, (1985) Ann. Rev. Genet. 19:423-462; Botstein & Shortle, Strategies and applications of in vitro mutagenesis, (1985) Science 229:1193-1201; Carter, Site-directed mutagenesis, (1986) Biochem. J. 237:1-7; and Kunkel, The efficiency of oligonucleotide directed mutagenesis, (1987) in Nucleic Acids & Molecular Biology (Eckstein, F. and Lilley, D. M. J. eds., Springer Verlag, Berlin)); mutagenesis using uracil containing templates (Kunkel, Rapid and efficient site-specific mutagenesis without phenotypic selection, (1985) Proc. Natl. Acad. Sci. USA 82:488-492; Kunkel et al., Rapid and efficient site-specific mutagenesis without phenotypic selection, (1987) Methods in Enzymol. 154, 367-382; and Bass et al., Mutant Trp repressors with new DNA-binding specificities, (1988) Science 242:240-245); oligonucleotide-directed mutagenesis ((1983) Methods in Enzymol. 100: 468-500; (1987) Methods in Enzymol. 154: 329-350; Zoller & Smith, Oligonucleotide-directed mutagenesis using M13-derived vectors: an efficient and general procedure for the production of point mutations in any DNA fragment, (1982) Nucleic Acids Res. 10:6487-6500; Zoller & Smith, Oligonucleotide-directed mutagenesis of DNA fragments cloned into M13 vectors, (1983) Methods in Enzymol. 100:468-500; and Zoller & Smith, Oligonucleotide-directed mutagenesis: a simple method using two oligonucleotide primers and a single-stranded DNA template, (1987) Methods in

Enzymol. 154:329-350); phosphorothioate-modified DNA mutagenesis (Taylor et al., The use of phosphorothioate-modified DNA in restriction enzyme reactions to prepare nicked DNA, (1985) Nucl. Acids Res. 13: 8749-8764; Taylor et al., The rapid generation of oligonucleotide-directed mutations at high frequency using phosphorothioate-modified DNA, (1985) Nucl. Acids Res. 13: 8765-8787; Nakamaye & Eckstein, Inhibition of restriction endonuclease Nci I cleavage by phosphorothioate groups and its application to oligonucleotide-directed mutagenesis, (1986) Nucl. Acids Res. 14: 9679-9698; Sayers et al., Y-T Exonucleases in phosphorothioate-based oligonucleotide-directed mutagenesis, (1988) Nucl. Acids Res. 16:791-802; and Sayers et al., Strand specific cleavage of phosphorothioate-containing DNA by reaction with restriction endonucleases in the presence of ethidium bromide, (1988) Nucl. Acids Res. 16: 803-814); mutagenesis using gapped duplex DNA (Kramer et al., The gapped duplex DNA approach to oligonucleotide-directed mutation construction, (1984) Nucl. Acids Res. 12: 9441-9456; Kramer & Fritz Oligonucleotide-directed construction of mutations via gapped duplex DNA, (1987) Methods in Enzymol. 154:350-367; Kramer et al., Improved enzymatic in vitro reactions in the gapped duplex DNA approach to oligonucleotide-directed construction of mutations, (1988) Nucl. Acids Res. 16: 7207; and Fritz et al., Oligonucleotide-directed construction of mutations: a gapped duplex DNA procedure without enzymatic reactions in vitro, (1988) Nucl. Acids Res. 16: 6987-6999).

Additional suitable methods include point mismatch repair (Kramer et al., Point Mismatch Repair, (1984) Cell 38:879-887), mutagenesis using repair-deficient host strains (Carter et al., Improved oligonucleotide site-directed mutagenesis using M13 vectors, (1985) Nucl. Acids Res. 13: 4431-4443; and Carter, Improved oligonucleotide-directed mutagenesis using M13 vectors, (1987) Methods in Enzymol. 154: 382-403), deletion mutagenesis (Eghtedarzadeh & Henikoff, Use of oligonucleotides to generate large deletions, (1986) Nucl. Acids Res. 14: 5115), restriction-selection and restriction-purification (Wells et al., Importance of hydrogen-bond formation in stabilizing the transition state of subtilisin, (1986) Phil. Trans. R. Soc. Lond. A 317: 415-423), mutagenesis by total gene synthesis (Nambiar et al., Total synthesis and cloning of a gene coding for the ribonuclease S protein, (1984) Science 223: 1299-1301; Sakamar and Khorana, Total synthesis and expression of a gene for the a-subunit of bovine rod outer segment guanine nucleotide-binding protein (transducin), (1988) Nucl. Acids Res. 14:

6361-6372; Wells et al., Cassette mutagenesis: an efficient method for generation of multiple mutations at defined sites, (1985) Gene 34:315-323; and Grundstrom et al., Oligonucleotide-directed mutagenesis by microscale ‘shot-gun’ gene synthesis, (1985) Nucl. Acids Res. 13: 3305-3316), double-strand break repair (Mandecki, Oligonucleotide-directed double-strand break repair in plasmids of Escherichia coli: a method for site-specific mutagenesis, (1986) Proc. Natl. Acad. Sci. USA, 83:7177-7181; and Arnold, Protein engineering for unusual environments, (1993) Current Opinion in Biotechnology 4:450-455). Additional details on many of the above methods can be found in Methods in Enzymology Volume 154, which also describes useful controls for trouble-shooting problems with various mutagenesis methods.

Additional details regarding various diversity generating methods can be found in the following U.S. patents, PCT publications and applications, and EPO publications: U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination;” WO 00/18906 by Patten et al., “Shuffling of Codon-Altered Genes;” WO 00/04190 by del Cardayre et al. “Evolution of Whole Cells and Organisms by Recursive Recombination;” WO 00/42561 by Crameri et al., “Oligonucleotide Mediated Nucleic Acid Recombination;” WO 00/42559 by Selifonov and Stemmer “Methods of Populating Data Structures for Use in Evolutionary Simulations;” WO 00/42560 by Selifonov et al., “Methods for Making Character Strings, Polynucleotides & Polypeptides Having Desired Characteristics;” WO 01/23401 by Welch et al., “Use of Codon-Varied Oligonucleotide Synthesis for Synthetic Shuffling;” and PCT/US01/06775 “Single-Stranded Nucleic Acid Template-Mediated Recombination and Nucleic Acid Fragment Isolation” by Affholter.

The polypeptides (e.g., avimers) used in the present invention are optionally expressed in cells. Multimer domains can be synthesized as a single protein using expression systems well known in the art. In addition to the many texts noted above, general texts which describe molecular biological techniques useful herein, including the use of vectors, promoters and many other topics relevant to expressing nucleic acids such as monomer domains, selected monomer domains, multimers and/or selected multimers, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”)). Examples of techniques sufficient to direct persons of skill through in vitro amplification methods, useful in identifying isolating and cloning monomer domains and multimers coding nucleic acids, including the polymerase chain reaction (PCR) the ligase chain reaction (LCR), Q-replicase amplification and other RNA polymerase mediated techniques (e.g., NASBA), are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369: 684-685 and the references therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase.

Vectors encoding, e.g., monomer domains and/or avimers may be introduced into host cells, produced and/or selected by recombinant techniques. Host cells are genetically engineered (i.e., transduced, transformed or transfected) with such vectors, which can be, for example, a cloning vector or an expression vector. The vector can be, for example, in the form of a plasmid, a viral particle, a phage, etc. The engineered host cells can be cultured in conventional nutrient media modified as appropriate for activating promoters, selecting transformants, or amplifying the monomer domain, selected monomer domain, multimer and/or selected multimer gene(s) of interest. The culture conditions, such as temperature, pH and the like, are those previously used with the host cell selected for expression, and will be apparent to those skilled in the art and in the references cited herein, including, e.g., Freshney (1994) Culture of Animal Cells, a Manual of Basic Technique, third edition, Wiley-Liss, New York and the references cited therein.

The polypeptides of the invention can also be produced in non-animal cells such as plants, yeast, fungi, bacteria and the like. Indeed, as noted throughout, phage display is an especially relevant technique for producing such polypeptides. In addition to Sambrook, Berger and Ausubel, details regarding cell culture can be found in Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla.

Avimers can also possess alterations of monomer domains, immuno-domains and/or multimers that improve pharmacological properties, reduce immunogenicity, or facilitate the transport of the multimer and/or monomer domain into a cell or tissue (e.g., through the blood-brain barrier, or through the skin). These types of alterations include a variety of modifications (e.g., the addition of sugar-groups or glycosylation), the addition of PEG, the addition of protein domains that bind a certain protein (e.g., HSA or other serum protein), the addition of proteins fragments or sequences that signal movement or transport into, out of and through a cell. Additional components can also be added to a multimer and/or monomer domain to manipulate the properties of the multimer and/or monomer domain. A variety of components can also be added including, e.g., a domain that binds a known receptor (e.g., a Fc-region protein domain that binds a Fc receptor), a toxin(s) or part of a toxin, a prodomain that can be optionally cleaved off to activate the multimer or monomer domain, a reporter molecule (e.g., green fluorescent protein), a component that bind a reporter molecule (such as a radionuclide for radiotherapy, biotin or avidin) or a combination of modifications.

As used herein, “directed evolution” refers to a process by which polynucleotide variants are generated, expressed, and screened for an activity (e.g., a polypeptide with binding activity for a human serum albumin target protein) in a recursive process. One or more candidates in the screen are selected and the process is then repeated using polynucleotides that encode the selected candidates to generate new variants. Directed evolution involves at least two rounds of variation generation and can include 3, 4, 5, 10, 20 or more rounds of variation generation and selection. Variation can be generated by any method known to those of skill in the art, including, e.g., by error-prone PCR, gene shuffling, chemical mutagenesis and the like.

The term “shuffling” is used herein to indicate recombination between non-identical sequences. In some embodiments, shuffling can include crossover via homologous recombination or via non-homologous recombination, such as via cre/lox and/or flp/frt systems. Shuffling can be carried out by employing a variety of different formats, including for example, in vitro and in vivo shuffling formats, in silico shuffling formats, shuffling formats that utilize either double-stranded or single-stranded templates, primer based shuffling formats, nucleic acid fragmentation-based shuffling formats, and oligonucleotide-mediated shuffling formats, all of which are based on recombination events between non-identical sequences and are described in more detail or referenced herein below, as well as other similar recombination-based formats.

The term “random” as used herein refers to a polynucleotide sequence or an amino acid sequence composed of two or more amino acids and constructed by a stochastic or random process. The random polynucleotide sequence or amino acid sequence can include framework or scaffolding motifs, which can comprise invariant sequences.

Groel and Groes

### Example 43

### Generation of Dual-Specific Ligand Comprising a Serum Albumin-Binding cpn10 (GroES) Non-Immunoglobulin Scaffold via CDR Grafting

The CDR3 domain of dAb7h14 is used to construct a cpn10 (GroES) non-immunoglobulin scaffold polypeptide that binds human serum albumin in the following manner. The CDR3 (AQGAALPRT; SEQ ID NO.:______) sequence of dAb7h14 is grafted into the cpn10 polypeptide in replacement of native cpn10 amino acid residues at positions 19-27 (mobile loop residues). Real-time binding analysis by BIAcore is performed to assess whether human serum albumin specifically binds to immobilized cpn10-derived polypeptide comprising the anti-human serum albumin CDR3 domain of dAb7h14. (One of skill in the art will recognize that binding affinity can be assessed using any appropriate method, including, e.g., precipitation of labeled human serum albumin, competitive BIAcore assay, etc.) If no or low human serum albumin affinity (e.g., Kd values in the μM range or higher) is detected, at least one of a number of strategies are employed to improve the human serum albumin binding properties of the CDR3-grafted cpn10 polypeptide, including any of the following methods that contribute to binding affinity.

The length of the dAb7h14 CDR3-grafted region of the cpn10 polypeptide corresponding to the mobile loop region within the native cpn10 polypeptide is adjusted through deletion of amino acid residues and/or the use of linker polypeptides. For example, the nine amino acid residue CDR3 peptide sequence of dAb7h14 is extended to 16 amino acid residues in length using amino acid linkers of, e.g., zero to seven residues in length located on either and/or both the N- or C-terminal flanks of the dAb7h14 CDR3 polypeptide sequence, thereby achieving a total grafted peptide sequence length of 16 amino acids within the CDR3-grafted domain corresponding to the mobile loop in the native cpn10 sequence. Such use of linker polypeptide(s) is optionally combined with mutagenesis of the linker sequences, CDR3 sequence(s) and/or non-CDR cpn10 sequences (e.g., using mutagenic optimization procedures as described below), in order to improve the human serum albumin binding capability of CDR3-grafted cpn10 polypeptides (e.g., via optimization of both CDR and fibronectin sequences within the CDR3-grafted cpn10 polypeptides). The polypeptide linkers employed for such purpose either possess a predetermined sequence, or, optionally, are selected from a population of randomized polypeptide linker sequences via assessment of the human serum albumin binding capabilities of linker-containing CDR3-grafted cpn10 polypeptides. Optimization methods are performed in parallel and/or iteratively. Both parallel and iterative optimization (e.g., affinity maturation) processes employ selection methods as described below and/or as known in the art as useful for optimization of polypeptide binding properties.

Human serum albumin binding of CDR-grafted cpn10 polypeptide(s) presenting dAb7h14 CDR3 is optimized via mutagenesis, optionally in combination with parallel and/or iterative selection methods as described below and/or as otherwise known in the art. Cpn10 scaffold polypeptide domains surrounding grafted dAb7h14 CDR3 polypeptide sequence are subjected to randomized and/or NNK mutagenesis, performed as described infra. Such mutagenesis is performed within the cpn10 polypeptide sequence upon non-grafted amino acid residues, and is optionally randomized in order to evolve new or improved human serum albumin-binding polypeptides. Optionally, the dAb7h14 CDR3 polypeptide domain presented within the CDR3-grafted cpn10 polypeptide is subjected to mutagenesis via, e.g., random mutagenesis, NNK mutagenesis, look-through mutagenesis and/or other art-recognized method. PCR is optionally used to perform such methods of mutagenesis, resulting in the generation of sequence diversity across targeted sequences within the CDR3-grafted cpn10 polypeptides. Such approaches are similar to those described infra for dAb library generation. In addition to random and/or look-through methods of mutagenesis, directed mutagenesis of targeted amino acid residues is employed where structural information establishes specific amino acid residues to be critical to binding of human serum albumin.

Cpn10 polypeptides comprising grafted dAb7h14 CDR3 sequence engineered as described above are subjected to parallel and/or iterative selection methods to identify those cpn10 polypeptides that are optimized for human serum albumin binding. For example, following production of a library of dAb7h14 CDR3-grafted cpn10 polypeptide sequences, this library of such polypeptides is displayed on phage and subjected to multiple rounds of selection requiring serum albumin binding and/or proliferation, as is described infra for selection of serum albumin-binding dAbs from libraries of dAbs. Optionally, selection is performed against serum albumin immobilized on immunotubes or against biotinlyated serum albumin in solution. Optionally, binding affinity is determined using surface plasmon resonance (SPR) and the BIAcore (Karlsson et al., 1991), using a BIAcore system (Uppsala, Sweden), with fully optimized monomeric and/or oligomeric cpn10-derived polypeptides ideally achieving human serum albumin binding affinity Kd values in the nM range or better.

Upon identification of monomeric cpn10-derived polypeptides that bind human serum albumin, human serum binding properties of such initial monomers may be further enhanced via combination of such monomers with other monomers, followed by further mutagenesis and/or selection, thereby forming an oligomeric cpn10/GroES composition possessing specific affinity for human serum albumin. Following identification of an oligomeric cpn10/GroES composition possessing affinity for human serum albumin, such polypeptides are then used to generate dual-specific ligand compositions by any of the methods described infra.

### Example 44

### Generation of Dual-Specific Ligand Comprising a Serum Albumin-Binding cpn10 Non-Immunoglobulin Scaffold via Selection of Serum Albumin Binding Moieties

The native cpn10 polypeptide is subjected to library selection and, optionally, affinity maturation techniques in order to produce human serum albumin-binding cpn10 non-immunoglobulin scaffold molecules for use in dual-specific ligands of the invention.

The capability of a native cpn10 polypeptide to bind human serum albumin is initially ascertained via BIAcore assay as described above. Following detection of no or low binding affinity (e.g., Kd values in the μM range or higher) of a cpn10 polypeptide for human serum albumin, at least one of a number of strategies are employed to impart human serum albumin binding properties to the cpn10 polypeptide, including one or more of the following methods that contribute to binding affinity.

Human serum albumin binding of cpn10 scaffold polypeptide(s) is achieved and optimized via mutagenic methods, optionally in combination with parallel and/or iterative selection methods as described below and/or as otherwise known in the art. Cpn10 scaffold polypeptide domains are subjected to randomized and/or NNK mutagenesis, performed as described infra. Such mutagenesis is performed upon the entirety of the cpn10 polypeptide or upon specific sequences within the cpn10 polypeptide, including mobile loop amino acid residues at positions 19-27, and is optionally randomized in order to evolve new or improved human serum albumin-binding polypeptides. PCR is optionally used to perform such methods of mutagenesis, resulting in the generation of sequence diversity across targeted sequences within the cpn10 polypeptides. (Such approaches are similar to those described infra for dAb library generation.) In addition to random methods of mutagenesis, directed mutagenesis of targeted amino acid residues is employed where structural information establishes specific amino acid residues of cpn10 polypeptides to be critical to binding of human serum albumin.

Cpn10 polypeptides engineered as described above are subjected to parallel and/or iterative selection methods to identify those cpn10 polypeptides that are optimized for human serum albumin binding. For example, following production of a library of mutagenized cpn10 polypeptide sequences, said library of polypeptides is displayed on phage and subjected to multiple rounds of selection requiring serum albumin binding and/or proliferation, as is described infra for selection of serum albumin-binding dAbs from libraries of dAbs. Optionally, selection is performed against serum albumin immobilized on immunotubes or against biotinlyated serum albumin in solution. Optionally, binding affinity is determined using surface plasmon resonance (SPR) and the BIAcore (Karlsson et al., 1991), using a BIAcore system (Uppsala, Sweden), with fully optimized monomeric and/or oligomeric cpn10-derived polypeptides ideally achieving human serum albumin binding affinity Kd values in the nM range or better.

Upon identification of monomeric cpn10-derived polypeptides that bind human serum albumin, human serum binding properties of such initial monomers may be further enhanced via combination of such monomers with other monomers, followed by further mutagenesis and/or selection, thereby forming an oligomeric cpn10/GroES composition possessing specific affinity for human serum albumin. Following identification of an oligomeric cpn10/GroES composition possessing affinity for human serum albumin, such polypeptides are then used to generate dual-specific ligand compositions by any of the methods described infra.

GroEL Polypeptides

GroEL is a key molecular chaperone in E. coli that consists of 14 subunits each of some 57.5 Kd molecular mass arranged in two seven membered rings (Braig et al. 1994 Proc. Natl. Acad. Sci. 90: 3978-3982). There is a large cavity in the GroEL ring system, and it is widely believed that the cavity is required for successful protein folding activity. For optimal activity, a co-chaperone, GroES, is required which consists of a seven membered ring of 10 Kd subunits (Hunt et al. 1996 Nature 379: 37-45). Each GroES subunit uses a mobile loop with a conserved hydrophobic tripeptide for interaction with GroEL (Landry et al. 1993 Nature 364: 255-258). The mobile loops are generally less than 16 amino acids in length and undergo a transition from disordered loops to β-hairpins concomitant with binding the apical domains of GroEL (Shewmaker et al. 2001 J. Biol. Chem. 276: 31257-31264). The activity of the GroEL/GroES complex requires ATP. GroEL and GroES are widespread throughout all organisms, and often referred to as chaperonin (cpn) molecules, cpn60 and cpn10, respectively.

GroEL is an allosteric protein. Allosteric proteins are a special class of oligomeric proteins, which alternate between two or more different three-dimensional structures during binding of ligands and substrates. Allosteric proteins are often involved in control processes in biology or where mechanical and physico-chemical energies are interconverted. The role of ATP is to trigger this allosteric change, causing GroEL to convert from a state that binds denatured proteins tightly to one that binds denatured proteins weakly. The co-chaperone, GroES, aids in this process by favoring the weak-binding state. It may also act as a cap, sealing off the cavity of GroEL. Further, its binding to GroEL is likely directly to compete with the binding of denatured substrates. The net result is that the binding of GroES and ATP to GroEL which has a substrate bound in its denatured form is to release the denatured substrate either into the cavity or into solution where it can refold.

GroEL and GroES are polypeptide scaffolds that can be used to multimerize monomeric polypeptides or protein domains, to produce multimeric proteins having any desired characteristic. As also described infra for, e.g., avimer compositions, it is often desirable to multimerize polypeptide monomers.

Many proteins require the assistance of molecular chaperones in order to be folded in vivo or to be refolded in vitro in high yields. Molecular chaperones are proteins, which are often large and require an energy source such as ATP to function. A key molecular chaperone in E. coli is GroEL, which consists of 14 subunits each of some 57.5 Kda molecular mass arranged in two seven membered rings. There is a large cavity in the GroEL ring system, and it is widely believed that the cavity is required for successful protein folding activity. For optimal activity, a co-chaperone, GroES, is required which consists of a seven membered ring of 10 Kda subunits. The activity of the GroEL/GroES complex requires energy source ATP.

Minichaperones have been described in detail elsewhere (see International patent application W099/05163, the disclosure of which in incorporated herein by reference). Minichaperone polypeptides possess chaperoning activity when in monomeric form and do not require energy in the form of ATP. Defined fragments of the apical domain of GroEL of approximately 143-186 amino acid residues in length have molecular chaperone activity towards proteins either in solution under monomeric conditions or when monodispersed and attached to a support.

The GroEL and/or GroES scaffolds allow for the oligomerisation of polypeptides to form functional protein oligomers which have activities which surpass those of recombinant monomeric polypeptides. Cpn10 is a widespread component of the cpn60/cpn10 chaperonin system. Examples of cpn10 include bacterial GroES and bacteriophage T4 Gp31, and are also listed below. Further members of the cpn10 family will be known to those skilled in the art.

Protein scaffold subunits assemble to form a protein scaffold. Such a scaffold may have any shape and may comprise any number of subunits. For certain GroEL and GroES embodiments, the scaffold comprises between 2 and 20 subunits, between 5 and 15 subunits, or about 10 subunits. The naturally-occurring scaffold structure of cpn10 family members comprises seven subunits, in the shape of a seven-membered ring or annulus. In certain embodiments, therefore, the scaffold is a seven-membered ring.

A heterologous amino acid sequence, which may be, e.g., a CDR3 domain derived from an antibody or antigen binding fragment thereof possessing affinity for a target protein (e.g., human serum albumin) or, optionally, which may be a single residue such as cysteine which allows for the linkage of further groups or molecules to the scaffold, can be inserted into the sequence of the oligomerisable protein scaffold subunit such that both the N- and C-termini of the polypeptide monomer are formed by the sequence of the oligomerisable protein scaffold subunit. Thus, the heterologous polypeptide is included with the sequence of the scaffold subunit, for example by replacing one or more amino acids thereof.

It is known that cpn10 subunits possess a “mobile loop” within their structure. The mobile loop is positioned between amino acids 15 and 34, preferably between amino acids 16 to 33, of the sequence of E. coli GroES, and equivalent positions on other members of the cpn10 family. The mobile loop of T4 Gp31 is located between residues 22 to 45, preferably 23 to 44. Optionally, the heterologous polypeptide can be inserted by replacing all or part of the mobile loop of a cpn10 family polypeptide. Where the protein scaffold subunit is a cpn10 family polypeptide, the heterologous sequence may moreover be incorporated at the N- or C-terminus thereof, or in positions which are equivalent to the roof b hairpin of cpn10 family peptides. This position is located between positions 54 and 67, preferably 55 to 66, and preferably 59 and 61 of bacteriophage T4 Gp31, or between positions 43 to 63, preferably 44 to 62, advantageously 50 to 53 of E. coli GroES.

Optionally, a polypeptide may be inserted at the N- or C-terminus of a scaffold subunit in association with circular permutation of the subunit itself. Circular permutation is described in Graf and Schachman, PNAS(USA) 1996, 93: 11591. Essentially, the polypeptide is circularized by fusion of the existing N- and C-termini, and cleavage of the polypeptide chain elsewhere to create novel N- and C-termini. In a preferred embodiment of the invention, the heterologous polypeptide may be included at the N- and/or C-terminus formed after circular permutation. The site of formation of the novel termini may be selected according to the features desired, and may include the mobile loop and/or the roof 13 hairpin.

Advantageously, heterologous sequences, which may be the same or different, may be inserted at more than one of the positions and/or at different positions than the above-identified positions within the protein scaffold subunit. Thus, each subunit may comprise two or more heterologous polypeptides, which are displayed on the scaffold when this is assembled. Heterologous polypeptides may be displayed on a scaffold subunit in free or constrained form, depending on the degree of freedom provided by the site of insertion into the scaffold sequence. For example, varying the length of the sequences flanking the mobile or β hairpin loops in the scaffold will modulate the degree of constraint of any heterologous polypeptide inserted therein.

GroEL and/or GroES compositions also may comprise a polypeptide oligomer comprising two or more monomers. The oligomer may be configured as a heterooligomer, comprising two or more different amino acid sequences inserted into the scaffold, or as a homooligomer, in which the sequences inserted into the scaffold are the same.

The monomers which constitute the oligomer may be covalently crosslinked to each other. Crosslinking may be performed by recombinant approaches, such that the monomers are expressed ab initio as an oligomer; alternatively, crosslinking may be performed at Cys residues in the scaffold. For example, unique Cys residues inserted between positions 50 and 53 of the GroES scaffold, or equivalent positions on other members of the cpn10 family, may be used to cross-link scaffold subunits.

The nature of the heterologous polypeptide inserted into the scaffold subunit may be selected at will. In certain embodiments, scaffold proteins are synthesized which display antibodies or fragments thereof such as scFv, natural or camelised VH domains and VH CDR3 fragments.

In an exemplary embodiment, a polypeptide monomer capable of oligomerisation can be prepared as described above and/or as set forth in WO 00/69907, incorporated herein by reference in its entirety. The method of such preparation can comprise insertion of a nucleic acid sequence encoding a heterologous polypeptide into a nucleic acid sequence encoding a subunit of an oligomerisable protein scaffold, incorporating the resulting nucleic acid into an expression vector, and expressing the nucleic acid to produce the polypeptide monomers. Optionally, a polypeptide oligomer may then be produced via a process that comprises allowing the polypeptide monomers produced as above to associate into an oligomer. In certain embodiments, the monomers are cross-linked to form the oligomer.

In certain embodiments, a scaffold polypeptide is based on members of the cpn10/Hsp10 family, such as GroES or an analogue thereof. A highly preferred analogue is the T4 polypeptide Gp31. GroES analogues, including Gp31, possess a mobile loop (Hunt, J. F., et al., (1997) Cell 90, 361-371; Landry, S. J., et al., (1996) Proc. Natl. Acad. Sci. U.S.A. 93, 11622-11627) which may be inserted into, or replaced, in order to fuse the heterologous polypeptide to the scaffold.

Cpn10 homologues are widespread throughout animals, plants and bacteria. For example, a search of GenBank indicates that cpn10 homologues are known in the following species: Actinobacillus actinomycetemcomitans; Actinobacillus pleuropneumoniae; Aeromonas salmonicida; Agrobacterium tumefaciens; Allochromatium vinosum; Amoeba proteus symbiotic bacterium; Aqui/ex aeolicus; Arabidopsis thaliana; Bacillus sp; Bacillus stearothermophilus; Bacillus subtilis; Bartonella henselae; Bordetella pertussis; Borrelia burgdorferi; Brucella abortus; Buchnera aphidicola; Burkholderia cepacia; Burkholderia vietnamiensis; Campylobacterjejuni; Caulobacter crescentus; Chlamydia muridarum; Chlamydia trachomatis; Chlamydophila pneumoniae; Clostridium acetobutylicum; Clostridium perfringens; Clostridium thermocellum; coliphage T-Cowdria ruminantium; Cyanelle Cyanophora paradoxa; Ehrlichia canis; Ehrlichia chaffeensis; Ehrlichia equi; Ehrlichia phagocytophila; Ehrlichia risticii; Ehrlichia sennetsu; Ehrlichia sp 'HGE agent; Enterobacter aerogenes; Enterobacter agglomerans; Enterobacter amnigenus; Enterobacter asburiae; Enterobacter gergoviae; Enterobacter intermedius; Erwinia aphidicola; Erwinia carotovora; Erwinia herbicola; Escherichia coli; Francisella tularensis; Glycine max; Haemophilus ducreyi; Haemophilus influenzae Rd; Helicobacter pylori; Holospora obtusa; Homo sapiens; Klebsiella ornithinolytica; Klebsiella oxytoca; Klebsiella planticola; Klebsiella pneumoniae; Lactobacillus helvetictis; LactobacillUS 7eae; Lactococcus lactis; Lawsonia intracellularis; Leptospira interrogans; Methylovorus sp strain SS; Mycobacterium avium; Mycobacterium avium subsp avium; Mycobacterium avium subsp paratuberculosis; Mycobacterium leprae; Mycobacterium tuberculosis; Mycoplasma genitalium; Mycoplasma pneumoniae; Myzus persicae primary endosymbiont; Neisseria gonorrhoeae; Oscillatoria sp NKBG,- Pantoea ananas; Pasteurella multocida; Porphyromonas gingivalis; Pseudomonas aeruginosa; Pseudomonas aeruginosa; Pseudomonas putida; Rattus norvegicus; Rattus norvegicus; Rhizobium leguminosarum; Rhodobacter capsulatus; Rhodobacter sphaeroides; Rhodothermus marinus; Rickettsia prowazekii; Rickettsia rickettsii; Saccharomyces cerevisiae; Serratia ficaria; Serratia marcescens; Serratia rubidaea; Sinorhizobium meliloti; Sitophilus oryzae principal endosymbiont; Stenotrophomonas maltophilia; Streptococcus pneumoniae; Streptomyces albus; Streptomyces coelicolor; Streptomyces coelicolor; Streptomyces lividans; Synechococcus sp; Synechococcus vulcanus; Synechocystis sp; Thermoanaerobacter brockii; Thermotoga maritima; Thermus aquaticus; Treponema pallidum; Wolbachia sp; Zymomonas mobilis.

An advantage of cpn10 family subunits is that they possess a mobile loop, responsible for the protein folding activity of the natural chaperonin, which may be removed without affecting the scaffold. Cpn10 with a deleted mobile loop possesses no biological activity, making it an advantageously inert scaffold, thus minimizing any potentially deleterious effects.

Insertion of an appropriate biologically active polypeptide can confer a biological activity (e.g., human serum albumin binding) on the novel polypeptide thus generated. Indeed, the biological activity of the inserted polypeptide may be improved by incorporation of the biologically active polypeptide into the scaffold, especially, e.g., when mutagenesis and affmity-based screening methods as described herein are used to optimize target protein binding of a scaffold-presented polypeptide.

Alternative sites for peptide insertion are possible. An advantageous option is in the position equivalent to the roof □hairpin in GroES. This involves replacement of Glu- in Gp31 by the desired peptide. The amino acid sequence is Pro (59)-Glu(60)-Gly(61). This is conveniently converted to a Smal site at the DNA level (CCC:GGG) encoding Pro-Gly, leaving a blunt-ended restriction site for peptide insertion as a DNA fragment. Similarly, an insertion may be made at between positions 50 and 53 of the GroES sequence, and at equivalent positions in other cpn10 family members. Alternatively, inverse PCR may be used, to display the peptide on the opposite side of the scaffold.

Members of the cpn60/Hsp60 family of chaperonin molecules may also be used as scaffolds. For example, the tetradecameric bacterial chaperonin GroEL may be used. In certain embodiments, heterologous polypeptides would be inserted between positions 191 and 376, in particular between positions 197 and 333 (represented by SacII engineered and unique Cla I sites) to maintain intact the hinge region between the equatorial and the apical domains in order to impart mobility to the inserted polypeptide. The choice of scaffold may depend upon the intended application of the oligomer (or dual-specific ligand comprising and/or derived from such an oligomer): for example, if the oligomer is intended for vaccination purposes, the use of an immunogenic scaffold, such as that derived from Mycobacterium tuberculosis, is highly advantageous and confers an adjuvant effect.

Mutants of cpn60 molecules can also be used. For example, the single ring mutant of GroEL (GroELSRI) contains four point mutations which effect the major attachment between the two rings of GroEL (R452E, E461A, S463A and V464A) and is functionally inactive in vitro because it is released to bind GroES. GroELSR2 has an additional mutation at Glu191-Gly, which restores activity by reducing the affinity for GroES. Both of these mutants form ring structures and would be suitable for use as scaffolds.

Certain naturally-occurring scaffold molecules are bacteriophage products: for this reason, naturally occurring antibodies to such scaffolds are rare. This enhances the use of scaffold fusions as vaccine agents. T4 Gp31 with a deleted loop has no biological activity (except as a dominant-negative or intracellular vaccine against T4 bacteriophage) thus minimizing deleterious effects on the host. However, insertion of appropriate sequences encoding polypeptides can confer biological activity on the novel proteins. Indeed, the biological activity may be improved by insertion into the scaffold protein.

The affinity of antibodies or antibody fragments for antigens (e.g., human serum albumins) may be increased by oligomerisation according to the present invention. Antibody fragments may be fragments such as Fv, Fab and F(ab′)2 fragments or any derivatives thereof, such as a single chain Fv fragments. The antibodies or antibody fragments may be non-recombinant, recombinant or humanized. The antibody may be of any immunoglobulin isotype, e.g., IgG, IgM, and so forth.

In certain embodiments, the antibody fragments may be camelised VH domains It is known that the main intermolecular interactions between antibodies and their cognate antigens are mediated through VH CDR3.

Use of GroEL and/or GroES (cpn10) scaffold molecules as described infra and as known in the art provides for the oligomerisation Of VH domains, or VH CDR3 domains, to produce a high-affinity oligomer. Two or more domains may be included in such an oligomer; in an oligomer based on a cpn10 scaffold, up to 7 domains may be included, forming a hetpameric oligomeric molecule (heptabody) that binds to a target protein (e.g., human serum albumin).

For purpose of imparting and/or optimizing the affinity of certain scaffold polypeptides/oligomers for a target protein (e.g., human serum albumin), variation may be introduced into heterologous polypeptides inserted into scaffold polypeptides, such that the specificity and/or affinity of such polypeptides/oligomers for their ligands/substrates can be examined and/or mapped. Variants may be produced of the same loop, or a set of standard different loops may be devised, in order to assess rapidly the affinity of a novel polypeptide for target protein (e.g., human serum albumin). Variants may be produced by randomization of sequences according to known techniques, such as PCR. They may be subjected to selection by a screening protocol, such as phage display, before incorporation into protein scaffolds.

An “oligomerisable scaffold”, as referred to herein, is a polypeptide which is capable of oligomerising or being oligomerised to form a scaffold and to which a heterologous polypeptide may be fused, preferably covalently, without abolishing the oligomerisation capabilities. Thus, it provides a “scaffold” using which polypeptides may be arranged into multimers in accordance with the present invention. Optionally, parts of the wild-type polypeptide from which the scaffold is derived may be removed, for example by replacement with the heterologous polypeptide which is to be presented on the scaffold.

Monomers are polypeptides which possess the potential to oligomerise or to be oligomerised. Oligomerisation can be brought about by the incorporation, in the polypeptide, of an oligomerisable scaffold subunit which will oligomerise with further scaffold subunits if combined therewith. Optionally, oligomerisation can be brought about via use of art-recognized linkers for purpose of joining together monomers.

As used herein, “oligomer” is synonymous with “polymer” or “multimer” and is used to indicate that the object in question is not monomeric. Thus, oligomeric polypeptides comprise at least two monomeric units joined together covalently or non-covalently. The number of monomeric units employed will depend on the intended use of the oligomer, and may be between 2 and 20 or more. Optionally, it is between 5 and 10, and preferably about 7.

Phage Display

Phage display technology has proved to be enormously useful in biological research. It enables ligands to be selected from large libraries of molecules. Scaffold technology can harness the power of phage display in a uniquely advantageous manner. Cpn10 molecules can be displayed as monomers on fd bacteriophages, similar to single-chain Fv molecule display. Libraries of insertions (in place of the highly mobile loop, e.g., using CDR3 polypeptides derived from human serum albumin-binding antibodies) are constructed by standard methods, and the resulting libraries screened for molecules of interest. Such selection is affinity-based. After identification of molecules that possess affinity for target protein (e.g., human serum albumin), potentially via one or more iterations of mutagenesis, expression (the GroEL proteins, −57.5 Kda GroEL and −10 Kda GroES, can be expressed and purified as previously described (Chatellier et al. 1998 Proc. Natl. Acad. Sci. USA 95: 9861-9866; Corrales and Fersht 1996 1: 265-273), or by any art-recognized method) and affinity screening, such molecules can be oligomerised, thereby taking advantage of the avidity of such molecules. Optionally, certain selected monomers will be able to crosslink or oligomerise their binding partners.

Fibronectin

### Example 45

### Generation of Dual-Specific Ligand Comprising a Serum Albumin-Binding Fibronectin Non-Immunoglobulin Scaffold via CDR Grafting

The CDR domains of dAb7h14 are used to construct a fibronectin non-immunoglobulin scaffold polypeptide that binds human serum albumin in the following manner. The CDR1 (RASQWIGSQLS; SEQ ID NO.: ______), CDR2 (WRSSLQS; SEQ ID NO.:______), and CDR3 (AQGAALPRT; SEQ ID NO.:______) sequences of dAb7h14 are grafted into ¹⁰Fn3 in replacement of native ¹⁰Fn3 amino acid residues at positions 21-31 (the BC loop), 51-56 (the DE loop), and 76-88 (the FG loop), respectively. Real-time binding analysis by BIAcore is performed to assess whether human serum albumin specifically binds to immobilized fibronectin-derived polypeptide comprising the anti-human serum albumin CDR domains of dAb7h14. (One of skill in the art will recognize that binding affinity can be assessed using any appropriate method, including, e.g., precipitation of labeled human serum albumin, competitive BIAcore assay, etc.) If no or low human serum albumin affinity (e.g., Kd values in the μM range or higher) is detected, at least one of a number of strategies are employed to improve the human serum albumin binding properties of the CDR-grafted fibronectin polypeptide, including any of the following methods that contribute to binding affinity.

The length(s) of dAb7h14 CDR-grafted regions of the fibronectin polypeptide corresponding to solvent-exposed loop regions within the native fibronectin polypeptide are adjusted through the use of linker polypeptides. For example, the nine amino acid residue CDR3 peptide sequence of dAb7h14 is extended to 13 amino acid residues in length using amino acid linkers of, e.g., zero to four residues in length located on either and/or both the N- or C-terminal flanks of the dAb7h14 CDR3 polypeptide sequence, thereby achieving a total grafted peptide sequence length of 13 amino acids within the CDR3-grafted domain corresponding to the FG loop in the native fibronectin sequence. Such use of linker polypeptide(s) is optionally combined with mutagenesis of the linker sequences, CDR sequences and/or non-CDR fibronectin sequences (e.g., using mutagenic optimization procedures as described below), in order to improve the human serum albumin binding capability of CDR-grafted fibronectin polypeptides (e.g., via optimization of both CDR and fibronectin sequences within the CDR-grafted fibronectin polypeptides). The polypeptide linkers employed for such purpose either possess a predetermined sequence, or, optionally, are selected from a population of randomized polypeptide linker sequences via assessment of the human serum albumin binding capabilities of linker-containing CDR-grafted fibronectin polypeptides. Optimization methods are performed in parallel and/or iteratively. Both parallel and iterative optimization (e.g., affinity maturation) processes employ selection methods as described below and/or as known in the art as useful for optimization of polypeptide binding properties.

Human serum albumin binding of CDR-grafted fibronectin polypeptide(s) presenting dAb7h14 CDRs is optimized via mutagenesis, optionally in combination with parallel and/or iterative selection methods as described below and/or as otherwise known in the art. ¹⁰Fn3 scaffold polypeptide domains surrounding grafted dAb7h14 CDR polypeptide sequences are subjected to randomized and/or NNK mutagenesis, performed as described infra. Such mutagenesis is performed within the ¹⁰Fn3 polypeptide sequence upon amino acids 1-9, 44-50, 61-54, 82-94 (edges of beta sheets); 19, 21, 30-46 (even), 79-65 (odd) (solvent-accessible faces of both beta sheets); and 14-16 and 36-45 (non-CDR-like solvent-accessible loops and beta turns), and is optionally randomized in order to evolve new or improved human serum albumin-binding polypeptides. Optionally, dAb7h14 CDR polypeptide domains presented within the CDR-grafted fibronectin polypeptide are subjected to mutagenesis via, e.g., random mutagenesis, NNK mutagenesis, look-through mutagenesis and/or other art-recognized method. PCR is optionally used to perform such methods of mutagenesis, resulting in the generation of sequence diversity across targeted sequences within the CDR-grafted fibronectin polypeptides. Such approaches are similar to those described infra for dAb library generation. In addition to random and/or look-through methods of mutagenesis, directed mutagenesis of targeted amino acid residues is employed where structural information establishes specific amino acid residues to be critical to binding of human serum albumin.

Fibronectin polypeptides comprising grafted dAb7h14 CDR sequences engineered as described above are subjected to parallel and/or iterative selection methods to identify those fibronectin polypeptides that are optimized for human serum albumin binding. For example, following production of a library of dAb7h14 CDR-grafted fibronectin polypeptide sequences, this library of such polypeptides is displayed on phage and subjected to multiple rounds of selection requiring serum albumin binding and/or proliferation, as is described infra for selection of serum albumin-binding dAbs from libraries of dAbs. Optionally, selection is performed against serum albumin immobilized on immunotubes or against biotinlyated serum albumin in solution. Optionally, binding affinity is determined using surface plasmon resonance (SPR) and the BIAcore (Karlsson et al., 1991), using a BIAcore system (Uppsala, Sweden), with fully optimized fibronectin-derived polypeptides ideally achieving human serum albumin binding affinity Kd values in the nM range or better. Following identification of fibronectin-derived polypeptides that bind human serum albumin, such polypeptides are then used to generate dual-specific ligand compositions by any of the methods described infra.

### Example 46

### Generation of Dual-Specific Ligand Comprising a Serum Albumin-Binding Fibronectin Non-Immunoglobulin Scaffold via Selection of Serum Albumin Binding Moieties

The native fibronectin protein—specifically the 10Fn3 polypeptide of fibronectin—is subjected to library selection and, optionally, affinity maturation techniques in order to produce human serum albumin-binding fibronectin non-immunoglobulin scaffold molecules for use in dual-specific ligands of the invention.

Real-time binding analysis by BIAcore is performed to assess whether human serum albumin specifically binds to immobilized native fibronectin and/or fibronectin-derived polypeptide. Following detection of no or low binding affinity (e.g., Kd values in the μM range or higher) of a fibronectin polypeptide for human serum albumin, at least one of a number of strategies are employed to impart human serum albumin binding properties to the fibronectin polypeptide, including one or more of the following methods that contribute to binding affinity.

Human serum albumin binding of fibronectin scaffold polypeptide(s) is achieved and optimized via mutagenic methods, optionally in combination with parallel and/or iterative selection methods as described below and/or as otherwise known in the art. ¹⁰Fn3 scaffold polypeptide domains are subjected to randomized and/or NNK mutagenesis, performed as described infra. Such mutagenesis is performed upon the entirety of the ¹⁰Fn3 polypeptide or upon specific sequences within the ¹⁰Fn3 polypeptide, including amino acids 1-9, 44-50, 61-54, 82-94 (edges of beta sheets); 19, 21, 30-46 (even), 79-65 (odd) (solvent-accessible faces of both beta sheets); 21-31, 51-56, 76-88 (CDR-like solvent-accessible loops); and 14-16 and 36-45 (other solvent-accessible loops and beta turns), and is optionally randomized in order to evolve new or improved human serum albumin-binding polypeptides. PCR is optionally used to perform such methods of mutagenesis, resulting in the generation of sequence diversity across targeted sequences within the fibronectin polypeptides. (Such approaches are similar to those described infra for dAb library generation.) In addition to random methods of mutagenesis, directed mutagenesis of targeted amino acid residues is employed where structural information establishes specific amino acid residues of fibronectin polypeptides to be critical to binding of human serum albumin.

Fibronectin polypeptides engineered as described above are subjected to parallel and/or iterative selection methods to identify those fibronectin polypeptides that are optimized for human serum albumin binding. For example, following production of a library of mutagenized fibronectin polypeptide sequences, said library of polypeptides is displayed on phage and subjected to multiple rounds of selection requiring serum albumin binding and/or proliferation, as is described infra for selection of serum albumin-binding dAbs from libraries of dAbs. Optionally, selection is performed against serum albumin immobilized on immunotubes or against biotinlyated serum albumin in solution. Optionally, binding affinity is determined using surface plasmon resonance (SPR) and the BIAcore (Karlsson et al., 1991), using a BIAcore system (Uppsala, Sweden), with fully optimized fibronectin-derived polypeptides ideally achieving human serum albumin binding affinity Kd values in the nM range or better.

Following identification of fibronectin polypeptides that bind human serum albumin, such polypeptides are then used to generate dual-specific ligand compositions by any of the methods described infra.

Fibronectin Non-Immunoglobulin Scaffolds

In certain embodiments of the invention, a non-immunoglobulin scaffold comprising fibronectin, or a functional moiety and/or fragment thereof, is engineered to bind serum albumin. A non-immunoglobulin scaffold structure derived from the fibronectin type III module (Fn3) is used. The fibronectin type III module is a common domain found in mammalian blood and structural proteins, that occurs more than 400 times in the protein sequence database and is estimated to occur in 2% of all proteins sequenced to date. Proteins that include an Fn3 module sequence include fibronectins, tenascin, intracellular cytoskeletal proteins, and prokaryotic enzymes (Bork and Doolittle, Proc. Natl. Acad. Sci. USA 89:8990, 1992; Bork et al., Nature Biotech. 15:553, 1997; Meinke et al., J. Bacteriol. 175:1910, 1993; Watanabe et al., J. Biol. Chem. 265:15659, 1990). A particular non-immunoglobulin scaffold of fibronectin is the tenth module of human Fn3 (¹⁰Fn3), which comprises 94 amino acid residues. The overall fold of this domain is closely related to that of the smallest functional antibody fragment, the variable region of the heavy chain, which comprises the entire antigen recognition unit in camel and llama IgG. The major differences between camel and llama domains and the ¹⁰Fn3 domain are that (i) ¹⁰Fn3 has fewer beta strands (seven vs. nine) and (ii) the two beta sheets packed against each other are connected by a disulfide bridge in the camel and llama domains, but not in ¹⁰Fn3.

The three loops of ¹⁰Fn3 corresponding to the antigen-binding loops of the IgG heavy chain run between amino acid residues 21-31 (BC), 51-56 (DE), and 76-88 (FG) (refer to FIG. 3 of U.S. Pat. No. 7,115,396, the complete contents of which are incorporated herein by reference). The lengths of the BC and DE loops, 11 and 6 residues, respectively, fall within the narrow range of the corresponding antigen-recognition loops found in antibody heavy chains, that is, 7-10 and 4-8 residues, respectively. Accordingly, a CDR grafting strategy can be readily employed to introduce heavy chain CDR sequences into these domains. Additionally and/or alternatively, these two loops can be subjected to introduction of genetic variability by any art-recognized method (e.g., site-directed, look-through or other mutagenesis method, randomization, etc.) and, optionally, the resulting polypeptide may be subjected to selection for high antigen affinity. (Alternatively, introduction of genetic variability and/or selection procedures can be used to identify compositions with lowered binding affinity and/or optimized properties such as stability, toxicity, etc.) Through use of such methods, the BC and DE loops of fibronectin can be engineered to make contacts with antigens equivalent to the contacts of the corresponding CDR1 and CDR2 domains in antibodies.

Unlike the BC and DE loops, the FG loop of ¹⁰Fn3 is 12 residues long, whereas the corresponding loop in antibody heavy chains ranges from 4-28 residues. Accordingly, to optimize antigen binding, the FG loop of ¹⁰Fn3 can be varied in length (e.g., via use of randomization and/or use of polypeptide linker sequences (which also can be randomized)) as well as in sequence to cover the CDR3 length range of 4-28 residues to obtain the greatest possible flexibility and affinity in antigen binding. Indeed, for both those methods in which CDRs are directly grafted into a fibronectin scaffold and those in which a native fibronectin scaffold is selected and/or optimized for binding of serum albumin (or other target antigen), the lengths as well as the sequences of the CDR-like loops of the antibody mimics may be randomized during in vitro or in vivo affinity maturation (as described in more detail below).

The tenth human fibronectin type III domain, ¹⁰Fn3, refolds rapidly even at low temperature; its backbone conformation has been recovered within 1 second at 5° C. Thermodynamic stability of ¹⁰Fn3 is high (ΔGᵤ=24 kJ/mol=5.7 kcal/mol), correlating with its high melting temperature of 110° C.

One of the physiological roles of ¹⁰Fn3 is as a subunit of fibronectin, a glycoprotein that exists in a soluble form in body fluids and in an insoluble form in the extracellular matrix (Dickinson et al., J. Mol. Biol. 236:1079, 1994). A fibronectin monomer of 220-250 Kd contains 12 type I modules, two type II modules, and 17 fibronectin type III modules (Potts and Campbell, Curr. Opin. Cell Biol. 6:648, 1994). Different type III modules are involved in the binding of fibronectin to integrins, heparin, and chondroitin sulfate. ¹⁰Fn3 was found to mediate cell adhesion through an integrin-binding Arg-Gly-Asp (RGD) motif on one of its exposed loops. Similar RGD motifs have been shown to be involved in integrin binding by other proteins, such as fibrinogen, von Wellebrand factor, and vitronectin (Hynes et al., Cell 69:11, 1992). No other matrix- or cell-binding roles have been described for ¹⁰Fn3.

The observation that ¹⁰Fn3 has only slightly more adhesive activity than a short peptide containing RGD is consistent with the conclusion that the cell-binding activity of ¹⁰Fn3 is localized in the RGD peptide rather than distributed throughout the ¹⁰Fn3 structure (Baron et al., Biochemistry 31:2068, 1992). The fact that ¹⁰Fn3 without the RGD motif is unlikely to bind to other plasma proteins or extracellular matrix makes ¹⁰Fn3 a useful scaffold to replace antibodies. In addition, the presence of ¹⁰Fn3 in natural fibrinogen in the bloodstream indicates that ¹⁰Fn3 itself is unlikely to be immunogenic in the organism of origin.

In addition, it was shown that the ¹⁰Fn3 framework possesses exposed loop sequences tolerant of randomization, facilitating the generation of diverse pools of antibody mimics. This determination was made by examining the flexibility of the ¹⁰Fn3 sequence. In particular, the human ¹⁰Fn3 sequence was aligned with the sequences of fibronectins from other sources as well as sequences of related proteins, and the results of this alignment were mapped onto the three-dimensional structure of the human ¹⁰Fn3 domain. This alignment revealed that the majority of conserved residues were found in the core of the beta sheet sandwich, whereas the highly variable residues were located along the edges of the beta sheets, including the N- and C-termini, on the solvent-accessible faces of both beta sheets, and on three solvent-accessible loops that served as the hypervariable loops for affinity maturation of the antibody mimics. In view of these results, the randomization of these three loops was determined to be unlikely to have an adverse effect on the overall fold or stability of the ¹⁰Fn3 framework itself.

For the human ¹⁰Fn3 sequence, this analysis indicated that, at a minimum, amino acids 1-9, 44-50, 61-54, 82-94 (edges of beta sheets); 19, 21, 30-46 (even), 79-65 (odd) (solvent-accessible faces of both beta sheets); 21-31, 51-56, 76-88 (CDR-like solvent-accessible loops); and 14-16 and 36-45 (other solvent-accessible loops and beta turns) could be randomized to evolve new or improved compound-binding proteins. In addition, as discussed above, alterations in the lengths of one or more solvent exposed loops could also be included in such directed evolution methods.

Alternatively, changes in the β-sheet sequences could also be used to evolve new proteins. These mutations change the scaffold and thereby indirectly alter loop structure(s). If this approach is taken, mutations should not saturate the sequence, but rather few mutations should be introduced. Preferably, no more than between 3-20 changes should be introduced to the n-sheet sequences by this approach.

Sequence variation can be introduced by any technique including, for example, mutagenesis by Taq polymerase (Tindall and Kunkel, Biochemistry 27:6008 (1988)), fragment recombination, or a combination thereof. Similarly, an increase of the structural diversity of libraries, for example, by varying the length as well as the sequence of the CDR-presenting and/or CDR-like loops, or by structural redesign based on the advantageous framework mutations found in selected pools, can be used to introduce further improvements in non-immunoglobulin scaffolds.

Fusion Proteins Comprising Fibronectin Scaffold Polypeptides

The fibronectin scaffold polypeptides described herein may be fused to other protein domains. For example, fibronectin scaffold polypeptides identified to bind human serum albumin can be fused with heavy chain single variable domains, or antigen binding fragments thereof, in order to generate a dual-specific ligand of the invention comprising a fibronectin-based serum albumin binding moiety. Fibronectin scaffold polypeptides additionally may be integrated with the human immune response by fusing the constant region of an IgG (F_{c}) with a fibronectin scaffold polypeptide, such as an ¹⁰Fn3 module, preferably through the C-terminus of ¹⁰Fn3. The F_{c} in such a ¹⁰Fn3-F_{c} fusion molecule activates the complement component of the immune response and can serve to increase the therapeutic value of the engineered fibronectin polypeptide. Similarly, a fusion between a fibronectin scaffold polypeptide, such as ¹⁰Fn3, and a complement protein, such as C1q, may be used to target cells, and a fusion between a fibronectin scaffold polypeptide, such as ¹⁰Fn3, and a toxin may be used to specifically destroy cells that carry a particular antigen. Any of these fusions may be generated by standard techniques, for example, by expression of the fusion protein from a recombinant fusion gene constructed using publicly available gene sequences and/or as otherwise described infra.

Scaffold Multimers

In addition to monomers, any of the fibronectin scaffold constructs described herein may be generated as dimers or multimers of scaffolds as a means to increase the valency and thus the avidity of antigen (e.g., serum albumin) binding. Such multimers may be generated through covalent binding. For example, individual 10Fn3 modules may be bound by imitating the natural 8Fn3-9Fn3-10Fn3 C-to-N-terminus binding or by imitating antibody dimers that are held together through their constant regions. A 10Fn3-Fc construct may be exploited to design dimers of the general scheme of 10Fn3-Fc::Fc-10Fn3. The bonds engineered into the Fc::Fc interface may be covalent or non-covalent. In addition, dimerizing or multimerizing partners other than Fc, such as other non-immunoglobulin scaffold moieties and/or immunoglobulin-based antigen-binding moieties, can be used in hybrids, such as 10Fn3 hybrids, to create such higher order structures. Other examples of multimers include single variable domains described herein.

In particular examples, covalently bonded multimers may be generated by constructing fusion genes that encode the multimer or, alternatively, by engineering codons for cysteine residues into monomer sequences and allowing disulfide bond formation to occur between the expression products. Non-covalently bonded multimers may also be generated by a variety of techniques. These include the introduction, into monomer sequences, of codons corresponding to positively and/or negatively charged residues and allowing interactions between these residues in the expression products (and therefore between the monomers) to occur. This approach may be simplified by taking advantage of charged residues naturally present in a monomer subunit, for example, the negatively charged residues of fibronectin. Another means for generating non-covalently bonded compositions comprising fibronectin scaffold polypeptides is to introduce, into the monomer gene (for example, at the amino- or carboxy-termini), the coding sequences for proteins or protein domains known to interact. Such proteins or protein domains include coil-coil motifs, leucine zipper motifs, and any of the numerous protein subunits (or fragments thereof) known to direct formation of dimers or higher order multimers.

Fibronectin-Like Molecules

Although ¹⁰Fn3 represents a preferred scaffold for the generation of antibody mimics, other molecules may be substituted for ¹⁰Fn3 in the molecules described herein. These include, without limitation, human fibronectin modules ¹Fn3-⁹Fn3 and ¹¹Fn3-¹⁷Fn3 as well as related Fn3 modules from non-human animals and prokaryotes. In addition, Fn3 modules from other proteins with sequence homology to ¹⁰Fn3, such as tenascins and undulins, may also be used. Other exemplary scaffolds having immunoglobulin-like folds (but with sequences that are unrelated to the V_{H} domain) include N-cadherin, ICAM-2, titin, GCSF receptor, cytokine receptor, glycosidase inhibitor, E-cadherin, and antibiotic chromoprotein. Further domains with related structures may be derived from myelin membrane adhesion molecule P0, CD8, CD4, CD2, class I MHC, T-cell antigen receptor, CD1, C2 and I-set domains of VCAM-1, I-set immunoglobulin domain of myosin-binding protein C, I-set immunoglobulin domain of myosin-binding protein H, I-set immunoglobulin domain of telokin, telikin, NCAM, twitchin, neuroglian, growth hormone receptor, erythropoietin receptor, prolactin receptor, GC-SF receptor, interferon-gamma receptor, β-galactosidase/glucuronidase, β-glucuronidase, and transglutaminase. Alternatively, any other protein that includes one or more immunoglobulin-like folds may be utilized. Such proteins may be identified, for example, using the program SCOP (Murzin et al., J. Mol. Biol. 247:536 (1995); Lo Conte et al., Nucleic Acids Res. 25:257 (2000).

Generally, any molecule that exhibits a structural relatedness to the V_{H} domain (as identified, for example, using the SCOP computer program above) can be utilized as a non-immunoglobulin scaffold. Such molecules may, like fibronectin, include three loops at the N-terminal pole of the molecule and three loops at the C-terminal pole, each of which may be randomized to create diverse libraries; alternatively, larger domains may be utilized, having larger numbers of loops, as long as a number of such surface randomizable loops are positioned closely enough in space that they can participate in antigen binding. Examples of polypeptides possessing more than three loops positioned close to each other include T-cell antigen receptor and superoxide dismutase, which each have four loops that can be randomized; and an Fn3 dimer, tissue factor domains, and cytokine receptor domains, each of which have three sets of two similar domains where three randomizable loops are part of the two domains (bringing the total number of loops to six).

In yet another alternative, any protein having variable loops positioned close enough in space may be utilized for candidate binding protein production. For example, large proteins having spatially related, solvent accessible loops may be used, even if unrelated structurally to an immunoglobulin-like fold. Exemplary proteins include, without limitation, cytochrome F, green fluorescent protein, GroEL, and thaumatin. The loops displayed by these proteins may be randomized and superior binders selected from a randomized library as described herein. Because of their size, molecules may be obtained that exhibit an antigen binding surface considerably larger than that found in an antibody-antigen interaction. Other useful scaffolds of this type may also be identified using the program SCOP (Murzin et al., J. Mol. Biol. 247: 536 (1995)) to browse among candidate proteins having numerous loops, particularly loops positioned among parallel beta sheets or a number of alpha-helices.

Modules from different organisms and parent proteins may be most appropriate for different applications. For example, in designing a fibronectin scaffold polypeptide of the invention, it may be most desirable to generate that protein from a fibronectin or fibronectin-like molecule native to the organism for which a therapeutic is intended. In contrast, the organism of origin is less important or even irrelevant for fibronectin scaffolds that are to be used for in vitro applications, such as diagnostics, or as research reagents.

For any of these molecules, libraries may be generated and used to select binding proteins by any of the methods described herein.

Directed Evolution of Scaffold-Based Binding Proteins

The non-immunoglobulin scaffolds described herein may be used in any technique for evolving new or improved binding proteins. In one particular example, the target of binding (e.g., serum albumin) is immobilized on a solid support, such as a column resin or microtiter plate well, and the target contacted with a library of candidate non-immunoglobulin scaffold-based binding proteins. Such a library may consist of fibronectin scaffold clones, such as ¹⁰Fn3 clones constructed from the native (wild type) ¹⁰Fn3 scaffold through randomization of the sequence and/or the length of the ¹⁰Fn3 CDR-like loops. If desired, this library may be an RNA-protein fusion library generated, for example, by the techniques described in Szostak et al., U.S. Ser. No. 09/007,005 and Ser. No. 09/247,190; Szostak et al., WO98/31700; and Roberts & Szostak, Proc. Natl. Acad. Sci. USA (1997) vol. 94, p. 12297-12302. Alternatively, it may be a DNA-protein library (for example, as described in Lohse, DNA-Protein Fusions and Uses Thereof, U.S. Ser. No. 60/110,549, U.S. Ser. No. 09/459,190, and WO 00/32823). The fusion library is incubated with the immobilized target, the support is washed to remove non-specific binders, and the tightest binders are eluted under very stringent conditions and subjected to PCR to recover the sequence information or to create a new library of binders which may be used to repeat the selection process, with or without further mutagenesis of the sequence. A number of rounds of selection may be performed until binders of sufficient affinity for the antigen (e.g., serum albumin) are obtained.

In one particular example, the ¹⁰Fn3 scaffold may be used as the selection target. For example, if a protein is required that binds a specific peptide sequence (e.g., serum albumin) presented in a ten residue loop, a single ¹⁰Fn3 clone is constructed in which one of its loops has been set to the length of ten and to the desired sequence. The new clone is expressed in vivo and purified, and then immobilized on a solid support. An RNA-protein fusion library based on an appropriate scaffold is then allowed to interact with the support, which is then washed, and desired molecules eluted and re-selected as described above.

Similarly, the scaffolds described herein, for example, the ¹⁰Fn3 scaffold, may be used to find natural proteins that interact with the peptide sequence displayed by the scaffold, for example, in an ¹⁰Fn3 loop. The scaffold protein, such as the ¹⁰Fn3 protein, is immobilized as described above, and an RNA-protein fusion library is screened for binders to the displayed loop. The binders are enriched through multiple rounds of selection and identified by DNA sequencing.

In addition, in the above approaches, although RNA-protein libraries represent exemplary libraries for directed evolution, any type of scaffold-based library may be used in the selection methods of the invention.

Use of Fibronectin Scaffold Polypeptides

The fibronectin scaffold polypeptides described herein may be evolved to bind serum albumin or any antigen of interest. Such fibronectin scaffold proteins have thermodynamic properties superior to those of natural antibodies and can be evolved rapidly in vitro. Accordingly, these fibronectin scaffold polypeptides may be employed to produce binding domains for use in the research, therapeutic, and diagnostic fields.

Mutagenic Affinity Maturation

The selections described herein may also be combined with mutagenesis after all or a subset of the selection steps to further increase library diversity. Methods of affinity maturation may employ, e.g., error-prone PCR (Cadwell and Joyce, PCR Methods Appl 2:28 (1992)) or alternative forms of random mutagenesis, NNK mutagenesis as described infra, look-through mutagenesis (wherein CDR-grafted fibronectin scaffold polypeptides are engineered to optimize antigen binding through use of naturally-occurring CDR diversity—refer, e.g., to WO 06/023144, incorporated herein by reference), and/or other art-recognized mutagenic approach for creating polypeptide diversity, that is combined with one or more rounds of selection for antigen-binding affinity.

Any of the scaffold proteins described infra may be combined with one another for use, e.g., in the dual-specific ligand compositions of the present invention. For example, CDRs may be grafted on to a CTLA-4 scaffold and used together with antibody VH or VL domains to form a multivalent ligand. Likewise, fibronectin, lipocalin, affibodies, and other scaffolds may be combined.

### Example 47

### Creation and Characterisation of Dual Specific scFv Antibodies (K8VK/VE2 and K8VK/VH4) Directed Against APS and -Gal and of a Dual Specific scFv Antibody (K8V#/VHC11) Directed Against BCL10 Protein and β-Gal

This example describes a method for making dual specific scFv antibodies (K8V/VH2 and K8VK/VH4) directed against APS and ss-gal and a dual specific scFv antibody (K8VK/VHC11) directed against BCL10 protein and β-gal, whereby a repertoire of VH variable domains linked to a germline (dummy) VK domain is first selected for binding to APS and BCL10 protein. The selected individual VH domains (VH2, VH4 and VHC11) are then combined with an individual β-gal binding VK domain (from K8scFv, Examples 1 and 2) and antibodies are tested for dual specificity.

A VH/dummy VKscFv library described in Example 1 was used to perform three rounds of selections on APS and two rounds of selections BCL10 protein. BCL10 protein is involved in the regulation of apoptosis and mutant forms of this protein are found in multiple tumour types, indicating that BCL10 may be commonly involved in the pathogenesis of human cancer (Willis et al., 1999).

In the case of APS the phage titres went up from 2. 8×105 in the first round to 8.0×108 in the third round. In the case of BCL10 the phage titres went up from 1.8×105 in the first round to 9.2×107 in the second round. The selections were performed as described in Example 1 using immunotubes coated with either APS or BCL10 at 100 μg/ml concentration.

To check for binding, 24 colonies from the third round of APS selections and 48 colonies from the second round of the BCL10 selections were screened by soluble scFv ELISA. A 96-well plate was coated with 100 pt1 of APS, BCL10, BSA, HSA and -gal atlOug/ml concentration in PBS overnight at 4 C. Production of the soluble scFv fragments was induced by IPTG as described by Harrison etal., (1996) and the supernatant (50 l) containing scFvs assayed directly. Soluble scFv ELISA was performed as described in Example 1 and the bound scFvs were detected with Protein L-HRP. Two clones (VH2 and VH4) were found to bind APS and one clone (VHC11) was specific for BCL10 (FIGS. 3, 47). No cross-reactivity with other proteins was observed.

To create dual specific antibodies each of these clones was digested with SalI/NotI to remove dummy VK chains and a SalI/Notl fragment containing (3-gal binding VK domain from K8scFv was ligated instead. The binding characteristics of the produced clones (K8VKNH2, K8VKNH4 and K8VK/VHC11) were tested in a soluble scFv ELISA as described above. All clones were found to be dual specific without any cross-reactivity with other proteins (FIG. 48).

### Example 48

### Creation and Characterisation of Single VH Domain Antibodies (Vg2sd and VH4sd) Directed Against APS

This example demonstrates that VH2 and VH4 variable domains directed against APS (described in Example 3) can bind this antigen in the absence of a complementary variable domain.

DNA preps of the scFv clones VH2 and VH4 (described in Example 3) were digested with Ncol/Xhol to cut out the VH domains (FIG. 2). These domains were then ligated into a Ncol/Xhol digested pITl vector (FIG. 2) to create VH single domain fusion with gene III.

The binding characteristics of the produced clones (VH2sd and VH4sd) were then tested by monoclonal phage ELISA. Phage particles were produced as described by Harrison etal., (1996). 96-well ELISA plates were coated with100 μl of APS, BSA, HSA, P-gal, ubiquitin, α-amylase and myosin at 10 μg/ml concentration in PBS overnight at 4° C. A standard ELISA protocol was followed (Hoogenboom etal., 1991) using detection of bound phage with anti-M13-HRP conjugate. ELISA results demonstrated that VH single domains specifically recognised APS when displayed on the surface of the filamentous bacteriophage (FIG. 49). The ELISA of soluble VH2sd and VH4sd gave the same results as the phage ELISA, indicating that these single domains are also able to recognise APS as soluble fragments (FIG. 50).

### Example 49

### Selection of Single VH Domain Antibodies Directed Against APS and Single VK Domain Antibodies Directed against p-Gal from a Repertoire of Single Antibody Domains

This example describes a method for making single VH domain antibodies directed against APS and single VK domain antibodies directed against β-gal by selecting repertoires of virgin single antibody variable domains for binding to these antigens in the absence of the complementary variable domains

Two human phage antibody libraries were used in this experiment.

Library 5 NNK VH single domain 4.08×10⁸

Library 6 NNK VK single domain 2.88×10⁸

The libraries are based on a single human framework for VH(V3-23/DP47 and JH4b) and V1c (012/02/DPK9 and Jκ1) with side chain diversity incorporated in complementarity determining regions (CDR2 and CDR3). VH sequence in Library 5 (complementary VH variable domain being absent) is diversified at positions H50, H52, H52a, H53, H55, H56,H58, H95, H96, H97 and H98 (NNK encoded). VK sequence in Library 6 (complementary VH variable domain being absent) is diversified at positions L50,L53, L91, L92,L93, L94 and L96 (NNK encoded) (FIG. 1). The libraries are in phagemid pIT1/single variable domain format (FIG. 2).

Two rounds of selections were performed on APS and β-gal using Library 5 and Library6, respectively. In the case of APS the phage titres went up from 9.2×105 in the first round to 1.1×10⁸ in the second round. In the case of β-gal the phage titres went up from 2.0×106 in the first round to 1.6×108 in the second round. The selections were performed as described in Example 1 using immunotubes coated with either APS or β-gal at 100 μg/ml concentration.

After second round 48 clones from each selection were tested for binding to their respective antigens in a soluble single domain ELISA. 96-well plates were coated with 100 μl of 10 μg/ml APS and BSA (negative control) for screening of the clones selected from Library 5 and with 100 μl or 10 μg/ml β-gal and BSA (negative control) for screening of the clones selected from Library 6. Production of the soluble VK and VH single domain fragments was induced by IPTG as described by Harrison etal., (1996) and the supernatant (50) containing single domains assayed directly. Soluble single domain ELISA was performed as soluble scFv ELISA described in Example 1 and the bound VK and VH single domains were detected with Protein L-HRP and Protein A-HRP, respectively. Five VH single domains (VHA10sd, VHA1sd, VHA5sd, VHC5sd and VHC11sd) selected from Library 5 were found to bind APS and one VK single domain (VκE5sd) selected from Library 6 was found to bind β-gal. None of the clones crossreacted with BSA (FIGS. 3, 11).

### Example 50

### Creation and Characterisation of the Dual Specific scFv Antibodies (VκE5/VH2 and VκE5/VH4) Directed Against APS and β-gal

This example demonstrates that dual specific scFv antibodies (VκE5/VH2 and VκE5/VH4) directed against APS and (3-gal could be created by combining VKE5sd variable domain that was selected for binding to ss-gal in the absence of a complementary variable domain (as described in Example 49) with VH2 and VH4 variable domains that were selected for binding to APS in the presence of the complementary variable domains (as described in Example 3).

To create these dual specific antibodies, pIT1 phagemid containing VκE5sd (Example 49) was digested with NcoI/XhoI (FIG. 2). NcoI/XhoI fragments containing VH variable domains from clones VH2 and VH4 (Example 3) were then ligated into the phagemid to create scFv clones VκE5/VH2 and VκE5/VH4, respectively.

The binding characteristics of the produced clones were tested in a soluble scFv ELISA.

A 96-well plate was coated with 100 μl of APS, β-gal and BSA (negative control) at 10 g/ml concentration in PBS overnight at 4 C. Production of the soluble scFv fragments was induced by IPTG as described by Harrison etal., (1996) and the supernatant (50 μl) containing scFvs assayed directly. Soluble scFv ELISA was performed as described in Example 1 and the bound scFvs were detected with Protein L-HRP. Both VκE5/VH2 and VκE5/VH4 clones were found to be dual specific. No cross-reactivity with BSA was detected (FIG. 52).

### Example 51

### Construction of Vectors for Converting the Existing scFv Dual Specific Antibodies into a Fab Format

a. Construction of the Cκ vector and Ck/gIII vector.

CK gene was PCR amplified from an individual clone A4 selected from a Fab library (Griffith et al., 1994) using CHBACKNOT as a 5′ (back) primer and CKSACFORFL as a 3′ (forward) primer (Table 1). 30 cycles of PCR amplification were performed as described by Ignatovich et al., (1997), except that Pfu polymerase was used as an enzyme. PCR product was digested with Notl/EcoRl and ligated into a NotI/EcoRI digested vector pHEN14Vκ (FIG. 53) to create a Cκ vector (FIG. 54).

Gene III was then PCR amplified from pIT2 vector (FIG. 2) using G3BACKSAC as a 5′(back) primer and LMB2 as a 3′ (forward) primer (Table 1). 30 cycles of PCR amplification were performed as above. PCR product was digested with SacI/EcoR and ligated into a SacI/EcoRI digested Cκ vector (FIG. 54) to create a Ck/gIII phagemid (FIG. 55).

b. Construction of the CH Vector.

CH gene was PCR amplified from an individual clone A4 selected from a Fab library (Griffith et al., 1994) using CHBACKNOT as a 5′ (back) primer and CHSACFOR as a 3′ (forward) primer (Table 1). 30 cycles of PCR amplification were performed as above.

PCR product was digested with NotI/BglII and ligated into a NotI/BgUI digested vector PACYC4VH (FIG. 16) to create a CH vector (FIG. 57).

### Example 52

### Construction of VκES/VH2 Fab Clone and Comparison of its Binding Properties with the VκE5/VH2 scFv Version (Example 6)

This example demonstrates that the dual specificity of the VE5/V2 scFv antibody is retained when the VK and VH variable domains are located on different polypeptide chains. Furthermore, the binding of the VE5/V2 Fab clone to ss-gal and APS becomes competitive. In contrast, VκE5/VH2 scFv antibody can bind to both antigens simultaneously.

To create a VκE5/VH2 Fab, DNA from VκE5/VH2 scFv clone was digested with SalI/NotI and the purified DNA fragment containing V#E5 variable domain was ligated into a SalI/NotI digested CK vector (FIG. 54). Ligation products were used to transform competent Escherichia coli TG-1 cells as described by Ignatovich et al., (1997) and thetransformants (VκE5/Cκ) were grown on TYE plates containing 1% glucose and 100 μg/ml ampicillin.

DNA from VκE5/VH2 scFv clone was also digested with SfiI/XhoI and the purified DNA fragment containing VH2 variable domain was ligated into a Sfil/XhoI digested CH vector (FIG. 57). Ligation products were used to transform competent E. coli TG-1 cells as above and the transformants (VH2/CH) were grown on TYE plates containing 1% glucose and 10 g/ml chloramphenicol.

DNA prep was then made form VκE5/CK clone and used to transform VH2/CH clone as described by Chung et al., (1989). Transformants were grown on TYE plates containing 1% glucose, 100 μg/ml ampicillin and 10 μg/ml chloramphenicol.

The clone containing both VκE5/Cκ and VH2/CH plasmids was then induced by IPTG to produce soluble VκE5/VH2 Fab fragments. Inductions were performed as described by Harrison et al., (1996), except that the clone was maintained in the media containing two antibiotics (100 μg/ml ampicillin and 10 μg/ml chloramphenicol) and after the addition of IPTG the temperature was kept at 25° C. overnight.

Binding of soluble VκE5/VH2 Fabs was tested by ELISA. A 96-well plate was coated with 100 μl of APS, β-gal and BSA (negative control) at 10 μg/ml concentration in PBS overnight at 4° C. Supernatant (50 μ) containing Fabs was assayed directly. Soluble Fab ELISA was performed as described in Example 1 and the bound Fabs were detected with Protein A-HRP. ELISA demonstrated the dual specific nature of VκE5NH2 Fab (FIG. 58).

The produced VκE5/VH2 Fab was also purified from 50 ml supernatant using Protein A Sepharose as described by Harlow & Lane (1988) and run on a non-reducing SDS-PAGE gel. Coomassie staining of the gel revealed a band of 50 kDa corresponding to a Fab fragment (data not shown).

A competition ELISA was then performed to compare VκE5/VH2 Fab and VκE5/VH2 scFv binding properties. A 96-well plate was coated with 100 μl of β-gal at 10 μg/ml concentration in PBS overnight at 4° C. A dilution of supernatants containing VκE5/VH2 Fab and VκE5/VH2 scFv was chosen such that OD 0.2 was achieved upon detection with Protein A-HRP. 50 μl of the diluted VκE5/VH2 Fab and VκE5/VH2 scFv supernatants were incubated for one hour at room temperature with 36, 72 and 180 μmoles of either native APS or APS that was denatured by heating to 70° C. for 10 minutes and then chilled immediately on ice. As a negative control, all of the diluted VκE5/VH2 Fab and VκE5/VH2 scFv supernatants were subjected to the same incubation with either native or denatured BSA. Following these incubations the mixtures were then put onto a β-gal coated ELISA plate and incubated for another hour. Bound VκE5/VH2 Fab and VκE5/VH2 scFv fragments were detected with Protein A-HRP.

ELISA demonstrated that VH2 variable domain recognises denatured form of APS (FIG. 19). This result was confirmed by BIAcore experiments when none of the constructs containing VH2 variable domain were able to bind to the APS coated chip (data not shown). ELISA also clearly showed that a very efficient competition was achieved with denatured APS for VκE5/VH2 Fab fragment, whereas in the case of VκE5/VH2 scFv binding to β-gal was not affected by competing antigen (FIG. 59). This could be explained by the fact that scFv represents a more open structure where V and VH variable domains can behave independently. Such freedom could be restricted in a Fab format.

All publications mentioned in the present specification, and references cited in said publications, are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

Annex 1; polypeptides which enhance half-life in vivo.

Alpha-1 Glycoprotein (Orosomucoid) (AAG)

Alpha-1 Antichyromotrypsin (ACT)

Alpha-1 Antitrypsin (AAT)

Alpha-1 Microglobulin (Protein HC) (AIM)

Alpha-2 Macroglobulin (A2M)

Antithrombin III (AT III)

Apolipoprotein A-1 (Apo A-1)

Apoliprotein B (Apo B)

Beta-2-microglobulin (B2M)

Ceruloplasmin (Cp)

Complement Component (C3)

Complement Component (C4)

C1 Esterase Inhibitor (C1 INH)

C-Reactive Protein (CRP)

Cystatin C (Cys C)

Ferritin (FER)

Fibrinogen (FIB)

Fibronectin (FN)

Haptoglobin (Hp)

Hemopexin (HPX)

Immunoglobulin A (IgA)

Immunoglobulin D (IgD)

Immunoglobulin E (IgE)

Immunoglobulin G (IgG)

Immunoglobulin M (IgM)

Immunoglobulin Light Chains (kapa/lambda)

Lipoprotein(a) [Lp(a)]

Mannose-bindign protein (MBP)

Myoglobin (Myo)

Plasminogen (PSM)

Prealbumin (Transthyretin) (PAL)

Retinol-binding protein (RBP)

Rheomatoid Factor (RF)

Serum Amyloid A (SAA)

Soluble Tranferrin Receptor (sTfR)

Transferrin (Tf)

Annex 2

| Pairing | Therapeutic relevant references. |
|---|---|
| TNF | TGF-b and TNF when injected into the ankle joint of collagen induced |
| ALPHA/TGF-β | arthritis model significantly enhanced joint inflammation. In non-collagen |
| | challenged mice there was no effect. |
| TNF ALPHA/IL-1 | TNF and IL-1 synergize in the pathology of uveitis. |
| | TNF and IL-1 synergize in the pathology of malaria (hypoglycaemia, NO). |
| | TNF and IL-1 synergize in the induction of polymorphonuclear (PMN) |
| | cells migration in inflammation. |
| | IL-1 and TNF synergize to induce PMN infiltration into the peritoneum. |
| | IL-1 and TNF synergize to induce the secretion of IL-1 by endothelial cells. |
| | Important in inflammation. |
| | IL-1 or TNF alone induced some cellular infiltration into knee synovium. |
| | IL-1 induced PMNs, TNF —monocytes. Together they induced a more |
| | severe infiltration due to increased PMNs. |
| | Circulating myocardial depressant substance (present in sepsis) is low |
| | levels of IL-1 and TNF acting synergistically. |
| TNF ALPHA/IL-2 | Most relating to synergisitic activation of killer T-cells. |
| TNF ALPHA/IL-3 | Synergy of interleukin 3 and tumor necrosis factor alpha in stimulating |
| | clonal growth of acute myelogenous leukemia blasts is the result of |
| | induction of secondary hematopoietic cytokines by tumor necrosis factor |
| | alpha. |
| | Cancer Res. 1992 Apr 15; 52(8): 2197-201. |
| TNF ALPHA/IL-4 | IL-4 and TNF synergize to induce VCAM expression on endothelial cells. |
| | Implied to have a role in asthma. Same for synovium - implicated in RA. |
| | TNF and IL-4 synergize to induce IL-6 expression in keratinocytes. |
| | Sustained elevated levels of VCAM-1 in cultured fibroblast-like |
| | synoviocytes can be achieved by TNF-alpha in combination with either IL- |
| | 4 or IL-13 through increased mRNA stability. Am J Pathol. 1999 |
| | Apr; 154(4): 1149-58 |
| TNF ALPHA/IL-5 | Relationship between the tumor necrosis factor system and the serum |
| | interleukin-4, interleukin-5, interleukin-8, eosinophil cationic protein, and |
| | immunoglobulin E levels in the bronchial hyperreactivity of adults and |
| | their children. Allergy Asthma Proc. 2003 Mar-Apr; 24(2): 111-8. |
| TNF ALPHA/IL-6 | TNF and IL-6 are potent growth factors for OH-2, a novel human myeloma |
| | cell line. Eur J Haematol. 1994 Jul; 53(1): 31-7. |
| TNF ALPHA/IL-8 | TNF and IL-8 synergized with PMNs to activate platelets. Implicated in |
| | Acute Respiratory Distress Syndrome. |
| | See IL-5/TNF (asthma). Synergism between interleukin-8 and tumor |
| | necrosis factor-alpha for neutrophil-mediated platelet activation. Eur |
| | Cytokine Netw. 1994 Sep-Oct; 5(5): 455-60. (adult respiratory distress |
| | syndrome (ARDS)) |
| TNF ALPHA/IL-9 | |
| TNF ALPHA/IL- | IL-10 induces and synergizes with TNF in the induction of HIV expression |
| 10 | in chronically infected T-cells. |
| TNF ALPHA/IL- | Cytokines synergistically induce osteoclast differentiation: support by |
| 11 | immortalized or normal calvarial cells. Am J Physiol Cell Physiol. 2002 |
| | Sep; 283(3): C679-87. (Bone loss) |
| TNF ALPHA/IL- | |
| 12 | |
| TNF ALPHA/IL- | Sustained elevated levels of VCAM-1 in cultured fibroblast-like |
| 13 | synoviocytes can be achieved by TNF-alpha in combination with either IL- |
| | 4 or IL-13 through increased mRNA stability. Am J Pathol. 1999 |
| | Apr; 154(4): 1149-58. |
| | Interleukin-13 and tumour necrosis factor-alpha synergistically induce |
| | eotaxin production in human nasal fibroblasts. Clin Exp Allergy. 2000 |
| | Mar; 30(3): 348-55. |
| | Interleukin-13 and tumour necrosis factor-alpha synergistically induce |
| | eotaxin production in human nasal fibroblasts. Clin Exp Allergy. 2000 |
| | Mar; 30(3): 348-55 (allergic inflammation) |
| | Implications of serum TNF-beta and IL-13 in the treatment response of |
| | childhood nephrotic syndrome. Cytokine. 2003 Feb 7; 21(3): 155-9. |
| TNF ALPHA/IL- | Effects of inhaled tumour necrosis factor alpha in subjects with mild |
| 14 | asthma. Thorax. 2002 Sep; 57(9): 774-8. |
| TNF ALPHA/IL- | Effects of inhaled tumour necrosis factor alpha in subjects with mild |
| 15 | asthma. Thorax. 2002 Sep; 57(9): 774-8. |
| TNF ALPHA/IL- | Tumor necrosis factor-alpha-induced synthesis of interleukin-16 in airway |
| 16 | epithelial cells: priming for serotonin stimulation. Am J Respir Cell Mol |
| | Biol. 2003 Mar; 28(3): 354-62. (airway inflammation) |
| | Correlation of circulating interleukin 16 with proinflammatory cytokines in |
| | patients with rheumatoid arthritis. Rheumatology (Oxford). 2001 |
| | Apr; 40(4): 474-5. No abstract available. |
| | Interleukin 16 is up-regulated in Crohn's disease and participates in TNBS |
| | colitis in mice. Gastroenterology. 2000 Oct; 119(4): 972-82. |
| TNF ALPHA/IL- | Inhibition of interleukin-17 prevents the development of arthritis in |
| 17 | vaccinated mice challenged with Borrelia burgdorferi. Infect Immun. 2003 |
| | Jun; 71(6): 3437-42. |
| | Interleukin 17 synergises with tumour necrosis factor alpha to induce |
| | cartilage destruction in vitro. Ann Rheum Dis. 2002 Oct; 61(10): 870-6. |
| | A role of GM-CSF in the accumulation of neutrophils in the airways caused |
| | by IL-17 and TNF-alpha. Eur Respir J. 2003 Mar; 21(3): 387-93. (Airway |
| | inflammation) |
| | Abstract Interleukin-1, tumor necrosis factor alpha, and interleukin-17 |
| | synergistically up-regulate nitric oxide and prostaglandin E2 production in |
| | explants of human osteoarthritic knee menisci. Arthritis Rheum. 2001 |
| | Sep; 44(9): 2078-83. |
| TNF ALPHA/IL- | Association of interleukin-18 expression with enhanced levels of both |
| 18 | interleukin-1beta and tumor necrosis factor alpha in knee synovial tissue of |
| | patients with rheumatoid arthritis. Arthritis Rheum. 2003 Feb; 48(2): 339-47. |
| | Abstract Elevated levels of interleukin-18 and tumor necrosis factor-alpha |
| | in serum of patients with type 2 diabetes mellitus: relationship with diabetic |
| | nephropathy. Metabolism. 2003 May; 52(5): 605-8. |
| TNF ALPHA/IL- | Abstract IL-19 induces production of IL-6 and TNF-alpha and results in |
| 19 | cell apoptosis through TNF-alpha. J Immunol. 2002 Oct 15; 169(8): 4288-97. |
| TNF ALPHA/IL- | Abstract Cytokines: IL-20 - a new effector in skin inflammation. Curr Biol. |
| 20 | 2001 Jul 10; 11(13): R531-4 |
| TNF | Inflammation and coagulation: implications for the septic patient. Clin |
| ALPHA/Complement | Infect Dis. 2003 May 15; 36(10): 1259-65. Epub 2003 May 08. Review. |
| TNF | MHC induction in the brain. |
| ALPHA/IFN-γ | Synergize in anti-viral response/IFN-β induction. |
| | Neutrophil activation/respiratory burst. |
| | Endothelial cell activation |
| | Toxicities noted when patients treated with TNF/IFN-γ as anti-viral therapy |
| | Fractalkine expression by human astrocytes. |
| | Many papers on inflammatory responses - i.e. LPS, also macrophage |
| | activation. |
| | Anti-TNF and anti-IFN-γ synergize to protect mice from lethal |
| | endotoxemia. |
| TGF-β/IL-1 | Prostaglndin synthesis by osteoblasts |
| | IL-6 production by intestinal epithelial cells (inflammation model) |
| | Stimulates IL-11 and IL-6 in lung fibroblasts (inflammation model) |
| | IL-6 and IL-8 production in the retina |
| TGF-β/IL-6 | Chondrocarcoma proliferation |
| IL-1/IL-2 | B-cell activation |
| | LAK cell activation |
| | T-cell activation |
| | IL-1 synergy with IL-2 in the generation of lymphokine activated killer |
| | cells is mediated by TNF-alpha and beta (lymphotoxin). Cytokine. 1992 |
| | Nov; 4(6): 479-87. |
| IL-1/IL-3 | |
| IL-1/IL-4 | B-cell activation |
| | IL-4 induces IL-1 expression in endothelial cell activation. |
| IL-1/IL-5 | |
| IL-1/IL-6 | B cell activation |
| | T cell activation (can replace accessory cells) |
| | IL-1 induces IL-6 expression |
| | C3 and serum amyloid expression (acute phase response) |
| | HIV expression |
| | Cartilage collagen breakdown. |
| IL-1/IL-7 | IL-7 is requisite for IL-1-induced thymocyte proliferation. Involvement of |
| | IL-7 in the synergistic effects of granulocyte-macrophage colony- |
| | stimulating factor or tumor necrosis factor with IL-1. J Immunol. 1992 Jan |
| | 1; 148(1): 99-105. |
| IL-1/IL-8 | |
| IL-1/IL-10 | |
| IL-1/IL-11 | Cytokines synergistically induce osteoclast differentiation: support by |
| | immortalized or normal calvarial cells. Am J Physiol Cell Physiol. 2002 |
| | Sep; 283(3): C679-87. (Bone loss) |
| IL-1/IL-16 | Correlation of circulating interleukin 16 with proinflammatory cytokines in |
| | patients with rheumatoid arthritis. Rheumatology (Oxford). 2001 |
| | Apr; 40(4): 474-5. No abstract available. |
| IL-1/IL-17 | Inhibition of interleukin-17 prevents the development of arthritis in |
| | vaccinated mice challenged with Borrelia burgdorferi. Infect Immun. 2003 |
| | Jun; 71(6): 3437-42. |
| | Contribution of interleukin 17 to human cartilage degradation and synovial |
| | inflammation in osteoarthritis. Osteoarthritis Cartilage. 2002 |
| | Oct; 10(10): 799-807. |
| | Abstract Interleukin-1, tumor necrosis factor alpha, and interleukin-17 |
| | synergistically up-regulate nitric oxide and prostaglandin E2 production in |
| | explants of human osteoarthritic knee menisci. Arthritis Rheum. 2001 |
| | Sep; 44(9): 2078-83. |
| IL-1/IL-18 | Association of interleukin-18 expression with enhanced levels of both |
| | interleukin-1beta and tumor necrosis factor alpha in knee synovial tissue of |
| | patients with rheumatoid arthritis. Arthritis Rheum. 2003 Feb; 48(2): 339-47. |
| IL-1/IFN-g | |
| IL-2/IL-3 | T-cell proliferation |
| | B cell proliferation |
| IL-2/IL-4 | B-cell proliferation |
| | T-cell proliferation |
| | (selectively inducing activation of CD8 and NK lymphocytes)IL-2R beta |
| | agonist P1-30 acts in synergy with IL-2, IL-4, IL-9, and IL-15: biological |
| | and molecular effects. J Immunol. 2000 Oct 15; 165(8): 4312-8. |
| IL-2/IL-5 | B-cell proliferation/Ig secretion |
| | IL-5 induces IL-2 receptors on B-cells |
| IL-2/IL-6 | Development of cytotoxic T-cells |
| IL-2/IL-7 | |
| IL-2/IL-9 | See IL-2/IL-4 (NK-cells) |
| IL-2/IL-10 | B-cell activation |
| IL-2/IL-12 | IL-12 synergizes with IL-2 to induce lymphokine-activated cytotoxicity |
| | and perforin and granzyme gene expression in fresh human NK cells. Cell |
| | Immunol. 1995 Oct 1; 165(1): 33-43. (T-cell activation) |
| IL-2/IL-15 | See IL-2/IL-4 (NK cells) |
| | (T cell activation and proliferation) IL-15 and IL-2: a matter of life and |
| | death for T cells in vivo. Nat Med. 2001 Jan; 7(1): 114-8. |
| IL-2/IL-16 | Synergistic activation of CD4+ T cells by IL-16 and IL-2. J Immunol. 1998 |
| | Mar 1; 160(5): 2115-20. |
| IL-2/IL-17 | Evidence for the early involvement of interleukin 17 in human and |
| | experimental renal allograft rejection. J Pathol. 2002 Jul; 197(3): 322-32. |
| IL-2/IL-18 | Interleukin 18 (IL-18) in synergy with IL-2 induces lethal lung injury in |
| | mice: a potential role for cytokines, chemokines, and natural killer cells in |
| | the pathogenesis of interstitial pneumonia. Blood. 2002 Feb 15; 99(4): 1289-98. |
| IL-2/TGF-β | Control of CD4 effector fate: transforming growth factor beta 1 and |
| | interleukin 2 synergize to prevent apoptosis and promote effector |
| | expansion. J Exp Med. 1995 Sep 1; 182(3): 699-709. |
| IL-2/IFN-γ | Ig secretion by B-cells |
| | IL-2 induces IFN-γ expression by T-cells |
| IL-2/IFN-α/β | None |
| IL-3/IL-4 | Synergize in mast cell growth |
| | Synergistic effects of IL-4 and either GM-CSF or IL-3 on the induction of |
| | CD23 expression by human monocytes: regulatory effects of IFN-alpha and |
| | IFN-gamma. Cytokine. 1994 Jul; 6(4): 407-13. |
| IL-3/IL-5 | |
| IL-3/IL-6 | |
| IL-3/IFN-γ | IL-4 and IFN-gamma synergistically increase total polymeric IgA receptor |
| | levels in human intestinal epithelial cells. Role of protein tyrosine kinases. |
| | J Immunol. 1996 Jun 15; 156(12): 4807-14. |
| IL-3/GM-CSF | Differential regulation of human eosinophil IL-3, IL-5, and GM-CSF |
| | receptor alpha-chain expression by cytokines: IL-3, IL-5, and GM-CSF |
| | down-regulate IL-5 receptor alpha expression with loss of IL-5 |
| | responsiveness, but up-regulate IL-3 receptor alpha expression. J Immunol. |
| | 2003 Jun 1; 170(11): 5359-66. (allergic inflammation) |
| IL-4/IL-2 | IL-4 synergistically enhances both IL-2- and IL-12-induced IFN-{gamma} |
| | expression in murine NK cells. Blood. 2003 Mar 13 [Epub ahead of print] |
| IL-4/IL-5 | Enhanced mast cell histamine etc. secretion in response to IgE |
| | A Th2-like cytokine response is involved in bullous pemphigoid. the role of |
| | IL-4 and IL-5 in the pathogenesis of the disease. Int J Immunopathol |
| | Pharmacol. 1999 May-Aug; 12(2): 55-61. |
| IL-4/IL-6 | |
| IL-4/IL-10 | |
| IL-4/IL-11 | Synergistic interactions between interleukin-11 and interleukin-4 in support |
| | of proliferation of primitive hematopoietic progenitors of mice. Blood. |
| | 1991 Sep 15; 78(6): 1448-51. |
| IL-4/IL-12 | Synergistic effects of IL-4 and IL-18 on IL-12-dependent IFN-gamma |
| | production by dendritic cells. J Immunol. 2000 Jan 1; 164(1): 64-71. |
| | (increase Th1/Th2 differentiation) |
| | IL-4 synergistically enhances both IL-2- and IL-12-induced IFN-{gamma} |
| | expression in murine NK cells. Blood. 2003 Mar 13 [Epub ahead of print] |
| IL-4/IL-13 | Abstract Interleukin-4 and interleukin-13 signaling connections maps. |
| | Science. 2003 Jun 6; 300(5625): 1527-8. (allergy, asthma) |
| | Inhibition of the IL-4/IL-13 receptor system prevents allergic sensitization |
| | without affecting established allergy in a mouse model for allergic asthma. |
| | J Allergy Clin Immunol. 2003 Jun; 111(6): 1361-1369. |
| IL-4/IL-16 | (asthma) Interleukin (IL)-4/IL-9 and exogenous IL-16 induce IL-16 |
| | production by BEAS-2B cells, a bronchial epithelial cell line. Cell |
| | Immunol. 2001 Feb 1; 207(2): 75-80 |
| IL-4/IL-17 | Interleukin (IL)-4 and IL-17 synergistically stimulate IL-6 secretion in |
| | human colonic myofibroblasts. Int J Mol Med. 2002 Nov; 10(5): 631-4. |
| | (Gut inflammation) |
| IL-4/IL-24 | IL-24 is expressed by rat and human macrophages. Immunobiology. 2002 |
| | Jul; 205(3): 321-34. |
| IL-4/IL-25 | Abstract New IL-17 family members promote Th1 or Th2 responses in the |
| | lung: in vivo function of the novel cytokine IL-25. J Immunol. 2002 Jul |
| | 1; 169(1): 443-53. (allergic inflammation) |
| | Abstract Mast cells produce interleukin-25 upon Fcepsilon RI-mediated |
| | activation. Blood. 2003 May 1; 101(9): 3594-6. Epub 2003 Jan 02. (allergic |
| | inflammation) |
| IL-4/IFN-γ | Abstract Interleukin 4 induces interleukin 6 production by endothelial cells: |
| | synergy with interferon-gamma. Eur J Immunol. 1991 Jan; 21(1): 97-101. |
| IL-4/SCF | Regulation of human intestinal mast cells by stem cell factor and IL-4. |
| | Immunol Rev. 2001 Feb; 179: 57-60. Review. |
| IL-5/IL-3 | Differential regulation of human eosinophil IL-3, IL-5, and GM-CSF |
| | receptor alpha-chain expression by cytokines: IL-3, IL-5, and GM-CSF |
| | down-regulate IL-5 receptor alpha expression with loss of IL-5 |
| | responsiveness, but up-regulate IL-3 receptor alpha expression. J Immunol. |
| | 2003 Jun 1; 170(11): 5359-66. (Allergic inflammation see abstract) |
| IL-5/IL-6 | |
| IL-5/IL-13 | Inhibition of allergic airways inflammation and airway |
| | hyperresponsiveness in mice by dexamethasone: role of eosinophils, IL-5, |
| | eotaxin, and IL-13. J Allergy Clin Immunol. 2003 May; 111(5): 1049-61. |
| IL-5/IL-17 | Interleukin-17 orchestrates the granulocyte influx into airways after |
| | allergen inhalation in a mouse model of allergic asthma. Am J Respir Cell |
| | Mol Biol. 2003 Jan; 28(1): 42-50. |
| IL-5/IL-25 | Abstract New IL-17 family members promote Th1 or Th2 responses in the |
| | lung: in vivo function of the novel cytokine IL-25. J Immunol. 2002 Jul |
| | 1; 169(1): 443-53. (allergic inflammation) |
| | Abstract Mast cells produce interleukin-25 upon Fcepsilon RI-mediated |
| | activation. Blood. 2003 May 1; 101(9): 3594-6. Epub 2003 Jan 02. (allergic |
| | inflammation) |
| IL-5/IFN-γ | |
| IL-5/GM-CSF | Differential regulation of human eosinophil IL-3, IL-5, and GM-CSF |
| | receptor alpha-chain expression by cytokines: IL-3, IL-5, and GM-CSF |
| | down-regulate IL-5 receptor alpha expression with loss of IL-5 |
| | responsiveness, but up-regulate IL-3 receptor alpha expression. J Immunol. |
| | 2003 Jun 1; 170(11): 5359-66. (Allergic inflammation) |
| IL-6/IL-10 | |
| IL-6/IL-11 | |
| IL-6/IL-16 | Interleukin-16 stimulates the expression and production of pro- |
| | inflammatory cytokines by human monocytes. Immunology. 2000 |
| | May; 100(1): 63-9. |
| IL-6/IL-17 | Stimulation of airway mucin gene expression by interleukin (IL)-17 |
| | through IL-6 paracrine/autocrine loop. J Biol Chem. 2003 May |
| | 9; 278(19): 17036-43. Epub 2003 Mar 06. (airway inflammation, asthma) |
| IL-6/IL-19 | Abstract IL-19 induces production of IL-6 and TNF-alpha and results in |
| | cell apoptosis through TNF-alpha. J Immunol. 2002 Oct 15; 169(8): 4288-97. |
| IL-6/IFN-g | |
| IL-7/IL-2 | Interleukin 7 worsens graft-versus-host disease. Blood. 2002 Oct |
| | 1; 100(7): 2642-9. |
| IL-7/IL-12 | Synergistic effects of IL-7 and IL-12 on human T cell activation. J |
| | Immunol. 1995 May 15; 154(10): 5093-102. |
| IL-7/IL-15 | Interleukin-7 and interleukin-15 regulate the expression of the bcl-2 and c- |
| | myb genes in cutaneous T-cell lymphoma cells. Blood. 2001 Nov |
| | 1; 98(9): 2778-83. (growth factor) |
| IL-8/IL-11 | Abnormal production of interleukin (IL)-11 and IL-8 in polycythaemia |
| | vera. Cytokine. 2002 Nov 21; 20(4): 178-83. |
| IL-8/IL-17 | The Role of IL-17 in Joint Destruction. Drug News Perspect. 2002 |
| | Jan; 15(1): 17-23. (arthritis) |
| | Abstract Interleukin-17 stimulates the expression of interleukin-8, growth- |
| | related oncogene-alpha, and granulocyte-colony-stimulating factor by |
| | human airway epithelial cells. Am J Respir Cell Mol Biol. 2002 |
| | Jun; 26(6): 748-53. (airway inflammation) |
| IL-8/GSF | Interleukin-8: an autocrine/paracrine growth factor for human |
| | hematopoietic progenitors acting in synergy with colony stimulating factor- |
| | 1 to promote monocyte-macrophage growth and differentiation. Exp |
| | Hematol. 1999 Jan; 27(1): 28-36. |
| IL-8/VGEF | Intracavitary VEGF, bFGF, IL-8, IL-12 levels in primary and recurrent |
| | malignant glioma. J Neurooncol. 2003 May; 62(3): 297-303. |
| IL-9/IL-4 | Anti-interleukin-9 antibody treatment inhibits airway inflammation and |
| | hyperreactivity in mouse asthma model. Am J Respir Crit Care Med. 2002 |
| | Aug 1; 166(3): 409-16. |
| IL-9/IL-5 | Pulmonary overexpression of IL-9 induces Th2 cytokine expression, |
| | leading to immune pathology. J Clin Invest. 2002 Jan; 109(1): 29-39. |
| | Th2 cytokines and asthma. Interleukin-9 as a therapeutic target for asthma. |
| | Respir Res. 2001; 2(2): 80-4. Epub 2001 Feb 15. Review. |
| | Abstract Interleukin-9 enhances interleukin-5 receptor expression, |
| | differentiation, and survival of human eosinophils. Blood. 2000 Sep |
| | 15; 96(6): 2163-71 (asthma) |
| IL-9/IL-13 | Anti-interleukin-9 antibody treatment inhibits airway inflammation and |
| | hyperreactivity in mouse asthma model. Am J Respir Crit Care Med. 2002 |
| | Aug 1; 166(3): 409-16. |
| | Direct effects of interleukin-13 on epithelial cells cause airway |
| | hyperreactivity and mucus overproduction in asthma. Nat Med. 2002 |
| | Aug; 8(8): 885-9. |
| IL-9/IL-16 | See IL-4/IL-16 |
| IL-10/IL-2 | The interplay of interleukin-10 (IL-10) and interleukin-2 (IL-2) in humoral |
| | immune responses: IL-10 synergizes with IL-2 to enhance responses of |
| | human B lymphocytes in a mechanism which is different from upregulation |
| | of CD25 expression. Cell Immunol. 1994 Sep; 157(2): 478-88. |
| IL-10/IL-12 | |
| IL-10/TGF-β | IL-10 and TGF-beta cooperate in the regulatory T cell response to mucosal |
| | allergens in normal immunity and specific immunotherapy. Eur J |
| | Immunol. 2003 May; 33(5): 1205-14. |
| IL-10/IFN-γ | |
| IL-11/IL-6 | Interleukin-6 and interleukin-11 support human osteoclast formation by a |
| | RANKL-independent mechanism. Bone. 2003 Jan; 32(1): 1-7. (bone |
| | resorption in inflammation) |
| IL-11/IL-17 | Polarized in vivo expression of IL-11 and IL-17 between acute and chronic |
| | skin lesions. J Allergy Clin Immunol. 2003 Apr; 111(4): 875-81. (allergic |
| | dermatitis) |
| | IL-17 promotes bone erosion in murine collagen-induced arthritis through |
| | loss of the receptor activator of NF-kappa B ligand/osteoprotegerin |
| | balance. J Immunol. 2003 Mar 1; 170(5): 2655-62. |
| IL-11/TGF-β | Polarized in vivo expression of IL-11 and IL-17 between acute and chronic |
| | skin lesions. J Allergy Clin Immunol. 2003 Apr; 111(4): 875-81. (allergic |
| | dermatitis) |
| IL-12/IL-13 | Relationship of Interleukin-12 and Interleukin-13 imbalance with class- |
| | specific rheumatoid factors and anticardiolipin antibodies in systemic lupus |
| | erythematosus. Clin Rheumatol. 2003 May; 22(2): 107-11. |
| IL-12/IL-17 | Upregulation of interleukin-12 and -17 in active inflammatory bowel |
| | disease. Scand J Gastroenterol. 2003 Feb; 38(2): 180-5. |
| IL-12/IL-18 | Synergistic proliferation and activation of natural killer cells by interleukin |
| | 12 and interleukin 18. Cytokine. 1999 Nov; 11(11): 822-30. |
| | Inflammatory Liver Steatosis Caused by IL-12 and IL-18. J Interferon |
| | Cytokine Res. 2003 Mar; 23(3): 155-62. |
| IL-12/IL-23 | nterleukin-23 rather than interleukin-12 is the critical cytokine for |
| | autoimmune inflammation of the brain. Nature. 2003 Feb |
| | 13; 421(6924): 744-8. |
| | Abstract A unique role for IL-23 in promoting cellular immunity. J Leukoc |
| | Biol. 2003 Jan; 73(1): 49-56. Review. |
| IL-12/IL-27 | Abstract IL-27, a heterodimeric cytokine composed of EBI3 and p28 |
| | protein, induces proliferation of naive CD4(+) T cells. Immunity. 2002 |
| | Jun; 16(6): 779-90. |
| IL-12/IFN-γ | IL-12 induces IFN-γ expression by B and T-cells as part of immune |
| | stimulation. |
| IL-13/IL-5 | See IL-5/IL-13 |
| IL-13/IL-25 | Abstract New IL-17 family members promote Th1 or Th2 responses in the |
| | lung: in vivo function of the novel cytokine IL-25. J Immunol. 2002 Jul |
| | 1; 169(1): 443-53. (allergic inflammation) |
| | Abstract Mast cells produce interleukin-25 upon Fcepsilon RI-mediated |
| | activation. Blood. 2003 May 1; 101(9): 3594-6. Epub 2003 Jan 02. (allergic |
| | inflammation) |
| IL-15/IL-13 | Differential expression of interleukins (IL)-13 and IL-15 in ectopic and |
| | eutopic endometrium of women with endometriosis and normal fertile |
| | women. Am J Reprod Immunol. 2003 Feb; 49(2): 75-83. |
| IL-15/IL-16 | IL-15 and IL-16 overexpression in cutaneous T-cell lymphomas: stage- |
| | dependent increase in mycosis fungoides progression. Exp Dermatol. 2000 |
| | Aug; 9(4): 248-51. |
| IL-15/IL-17 | Abstract IL-17, produced by lymphocytes and neutrophils, is necessary for |
| | lipopolysaccharide-induced airway neutrophilia: IL-15 as a possible trigger. |
| | J Immunol. 2003 Feb 15; 170(4): 2106-12. (airway inflammation) |
| IL-15/IL-21 | IL-21 in Synergy with IL-15 or IL-18 Enhances IFN-gamma Production in |
| | Human NK and T Cells. J Immunol. 2003 Jun 1; 170(11): 5464-9. |
| IL-17/IL-23 | Interleukin-23 promotes a distinct CD4 T cell activation state characterized |
| | by the production of interleukin-17. J Biol Chem. 2003 Jan |
| | 17; 278(3): 1910-4. Epub 2002 Nov 03 |
| IL-17/TGF-β | Polarized in vivo expression of IL-11 and IL-17 between acute and chronic |
| | skin lesions. J Allergy Clin Immunol. 2003 Apr; 111(4): 875-81. (allergic |
| | dermatitis) |
| IL-18/IL-12 | Synergistic proliferation and activation of natural killer cells by interleukin |
| | 12 and interleukin 18. Cytokine. 1999 Nov; 11(11): 822-30. |
| | Abstract Inhibition of in vitro immunoglobulin production by IL-12 in |
| | murine chronic graft-vs.-host disease: synergism with IL-18. Eur J |
| | Immunol. 1998 Jun; 28(6): 2017-24. |
| IL-18/IL-21 | IL-21 in Synergy with IL-15 or IL-18 Enhances IFN-gamma Production in |
| | Human NK and T Cells. J Immunol. 2003 Jun 1; 170(11): 5464-9. |
| IL-18/TGF-β | Interleukin 18 and transforming growth factor betal in the serum of |
| | patients with Graves' ophthalmopathy treated with corticosteroids. Int |
| | Immunopharmacol. 2003 Apr; 3(4): 549-52. |
| IL-18/IFN-γ | |
| Anti-TNF | Synergistic therapeutic effect in DBA/1 arthritic mice. |
| ALPHA/anti-CD4 | |

Annex 3: Oncology combinations

| Target | Disease | Pair with |
|---|---|---|
| CD89* | Use as cytotoxic cell recruiter | all |
| CD19 | B cell lymphomas | HLA-DR |
| | | CD5 |
| HLA-DR | B cell lymphomas | CD89 |
| | | CD19 |
| | | CD5 |
| CD38 | Multiple myeloma | CD138 |
| | | CD56 |
| | | HLA-DR |
| CD138 | Multiple myeloma | CD38 |
| | | CD56 |
| | | HLA-DR |
| CD138 | Lung cancer | CD56 |
| | | CEA |
| CD33 | Acute myelod lymphoma | CD34 |
| | | HLA-DR |
| CD56 | Lung cancer | CD138 |
| | | CEA |
| CEA | Pan carcinoma | MET receptor |
| VEGF | Pan carcinoma | MET receptor |
| VEGF | Pan carcinoma | MET receptor |
| receptor | | |
| IL-13 | Asthma/pulmonary | IL-4 |
| | inflammation | IL-5 |
| | | Eotaxin(s) |
| | | MDC |
| | | TARC |
| | | TNFα |
| | | IL-9 |
| | | EGFR |
| | | CD40L |
| | | IL-25 |
| | | MCP-1 |
| | | TGFβ |
| IL-4 | Asthma | IL-13 |
| | | IL-5 |
| | | Eotaxin(s) |
| | | MDC |
| | | TARC |
| | | TNFα |
| | | IL-9 |
| | | EGFR |
| | | CD40L |
| | | IL-25 |
| | | MCP-1 |
| | | TGFβ |
| Eotaxin | Asthma | IL-5 |
| | | Eotaxin-2 |
| | | Eotaxin-3 |
| EGFR | cancer | HER2/neu |
| | | HER3 |
| | | HER4 |
| HER2 | cancer | HER3 |
| | | HER4 |
| TNFR1 | RA/Crohn's disease | IL-1R |
| | | IL-6R |
| | | IL-18R |
| TNFα | RA/Crohn's disease | IL-1α/β |
| | | IL-6 |
| | | IL-18 |
| | | ICAM-1 |
| | | IL-15 |
| | | IL-17 |
| IL-1R | RA/Crohn's disease | IL-6R |
| | | IL-18R |
| IL-18R | RA/Crohn's disease | IL-6R |

Annex 4
Data Summary

| | | Equilibrium dissocation | | | ND50 for cell based |
|---|---|---|---|---|---|
| TARGET | dAb | constant (Kd = Koff/Kon) | Koff | IC50 for ligand assay | neutralisn assay |
| TAR1 | TAR1 | 300 nM to 5 pM | 5 × 10⁻¹ to 1 × 10⁻⁷ | 500 nM to 100 pM | 500 nM to 50 pM |
| | monomers | (ie, 3 × 10⁻⁷ to | | | |
| | | 5 × 10⁻¹²), preferably | | | |
| | | 50 nM to 20 pM | | | |
| | TAR1 dimers | As TAR1 monomer | As TAR1 monomer | As TAR1 monomer | As TAR1 monomer |
| | TAR1 trimers | As TAR1 monomer | As TAR1 monomer | As TAR1 monomer | As TAR1 monomer |
| | TAR1-5 | | | | |
| | TAR1-27 | | | | |
| | TAR1-5-19 | 30 nM | | | |
| | monomer | | | | |
| | TAR1-5-19 | | | With (Gly₄Ser)₃ linker = 20 nm | =30 nM |
| | homodimer | | | With (Gly₄Ser)₅ linker = 2 nm | =3 nM |
| | | | | With (Gly₄Ser)₇ linker = 10 nm | =15 nM |
| | | | | In Fab format = 1 nM | |
| | TAR1-5-19 | | | With (Gly₄Ser)ₙ linker | |
| | heterodimers | | | TAR1-5-19 d2 = 2 nM | |
| | | | | TAR1-5-19 d3 = 8 nM | |
| | | | | TAR1-5-19 d4 = 2-5 nM | =12 nM |
| | | | | TAR1-5-19 d5 = 8 nM | =10 nM |
| | | | | In Fab format | |
| | | | | TAR1-5-19CH d1CK = 6 nM | |
| | | | | TAR1-5-19CK d1CH = 6 nM | |
| | | | | TAR1-5-19CH d2CK = 8 nM | |
| | | | | TAR1-5-19CH d3CK = 3 nM | =12 nM |
| | TAR1-5 | | | With (Gly₄Ser)ₙ linker | |
| | heterodimers | | | TAR1-5d1 = 30 nM | |
| | | | | TAR1-5d2 = 50 nM | |
| | | | | TAR1-5d3 = 300 nM | |
| | | | | TAR1-5d4 = 3 nM | |
| | | | | TAR1-5d5 = 200 nM | |
| | | | | TAR1-5d6 = 100 nM | |
| | | | | In Fab format | |
| | | | | TAR1-5CH d2CK = 30 nM | =60 nM |
| | | | | TAR1-5CK d3HH = 100 nM | |
| | TAR1-5-19 | | | 0.3 nM | 3-10 nM (eg, 3 nM) |
| | homotrimer | | | | |
| TAR2 | TAR2 | As TAR1 monomer | As TAR1 monomer | 500 nM to 100 pM | 500 nM to 50 pM |
| | monomers | | | | |
| | TAR2-10 | | | | |
| | TAR2-5 | | | | |
| Serum | Anti-SA | 1 nM to 500 μM, | | 1 nM to 500 μM, | |
| Albumin | monomers | preferably 100 nM to 10 μM | | preferably 100 nM to 10 μM | |
| | | In Dual Specific format, | | In Dual Specific format, target | |
| | | target affinity is 1 to | | affinity is 1 to 100,000 × affinity | |
| | | 100,000 × affinity of SA | | of SA dAb affinity, eg 100 pM | |
| | | dAb affinity, eg 100 pM | | (target) and 10 μM SA affinity. | |
| | | (target) and 10 μM SA | | | |
| | | affinity. | | | |
| | MSA-16 | 200 nM | | | |
| | MSA-26 | 70 nM | | | |

1. (canceled)

2. A ligand comprising a single variable domain, wherein the single variable domain specifically binds to an antigen, and the variable domain comprises a Kd for the antigen of 300 nM to 5 pM.

3. The ligand of claim 2, wherein the variable domain comprises a Kd for the antigen of 50 nM to 20 pM.

4. The ligand of claim 2, wherein the antigen is selected from the group consisting of wherein said first single variable domain specifically binds to an antigen selected from the group consisting of, human, protein, animal protein, cytokine, cytokine receptor, ApoE, Apo-SAA, BDNF, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER 3 and HER 4, CD4, human chemokine receptors CXCR4 or CCR5, non-structural protein type 3 (NS3) from the hepatitis C virus, TNF-α, IgE, IFN-gamma, MMP-12, CEA, H. pylori, TB, influenza, Hepatitis E, MMP-12, epidermal growth factor receptor (EGFR), ErBb2 receptor on tumor cells, LDL receptor, FGF2 receptor, ErbB2 receptor, transferrin receptor, PDGF receptor, VEGF receptor, PsmAr, an extracellular matrix protein, elastin, fibronectin, laminin, α1-antitrypsin, tissue factor protease inhibitor, PDK1, GSK1, Bad, caspase-9, Forkhead, an antigen of Helicobacter pylori, an antigen of Mycobacterium tuberculosis, an antigen of influenza virus, and serum albumin.

5. A ligand comprising a single variable domain, wherein the single variable domain specifically binds to an antigen, and the variable domain comprises a K_{off} for the antigen of 5×10⁻¹ to 1×10⁻⁷ S⁻¹.

6. The ligand of claim 5, wherein variable domain comprises a a K_{off} for the antigen of 1×10⁻² to 1×10⁻⁶ S⁻¹.

7. The ligand of claim 5, wherein the second antigen is selected from the group consisting of wherein said first single variable domain specifically binds to an antigen selected from the group consisting of, human, protein, animal protein, cytokine, cytokine receptor, ApoE, Apo-SAA, BDNF, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL- IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER 3 and HER 4, CD4, human chemokine receptors CXCR4 or CCR5, non-structural protein type 3 (NS3) from the hepatitis C virus, TNF-α, IgE, IFN-gamma, MMP-12, CEA, H. pylori, TB, influenza, Hepatitis E, MMP-12, epidermal growth factor receptor (EGFR), ErBb2 receptor on tumor cells, LDL receptor, FGF2 receptor, ErbB2 receptor, transferrin receptor, PDGF receptor, VEGF receptor, PsmAr, an extracellular matrix protein, elastin, fibronectin, laminin, α1-antitrypsin, tissue factor protease inhibitor, PDK1, GSK1, Bad, caspase-9, Forkhead, an antigen of Helicobacter pylori, an antigen of Mycobacterium tuberculosis, an antigen of influenza virus, and serum albumin.

8. A ligand comprising a single variable domain, wherein the single variable domain specifically binds to an antigen, and the variable domain comprises a half life of at least 12 hours in mammalian serum.

9. The ligand of claim 8, wherein the single variable domain comprises a half life of at least 24 hours in mammalian serum.

10. The ligand of claim 8, wherein the second antigen is selected from the group consisting of wherein said first single variable domain specifically binds to an antigen selected from the group consisting of, human, protein, animal protein, cytokine, cytokine receptor, ApoE, Apo-SAA, BDNF, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL- IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER 3 and HER 4, CD4, human chemokine receptors CXCR4 or CCR5, non-structural protein type 3 (NS3) from the hepatitis C virus, TNF-α, IgE, IFN-gamma, MMP-12, CEA, H. pylori, TB, influenza, Hepatitis E, MMP-12, epidermal growth factor receptor (EGFR), ErBb2 receptor on tumor cells, LDL receptor, FGF2 receptor, ErbB2 receptor, transferrin receptor, PDGF receptor, VEGF receptor, PsmAr, an extracellular matrix protein, elastin, fibronectin, laminin, α1-antitrypsin, tissue factor protease inhibitor, PDK1, GSK1, Bad, caspase-9, Forkhead, an antigen of Helicobacter pylori, an antigen of Mycobacterium tuberculosis, an antigen of influenza virus, and serum albumin.

11. A dual specific ligand comprising a first single variable domain and a second a single variable domain where at least one of the first single variable domain and the second single variable domain comprises a Kd for the antigen of 300 nM to 5 pM.

12. The ligand of claim 11, wherein the variable domain comprises a Kd for the antigen of 50 nM to 20 pM.

13. The dual specific ligand of claim 11, wherein said first single variable domain specifically binds to an antigen selected from the group consisting of, human, protein, animal protein, cytokine, cytokine receptor, ApoE, Apo-SAA, BDNF, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER 3 and HER 4, CD4, human chemokine receptors CXCR4 or CCR5, non-structural protein type 3 (NS3) from the hepatitis C virus, TNF-α, IgE, IFN-gamma, MMP-12, CEA, H. pylori, TB, influenza, Hepatitis E, MMP-12, epidermal growth factor receptor (EGFR), ErBb2 receptor on tumor cells, LDL receptor, FGF2 receptor, ErbB2 receptor, transferrin receptor, PDGF receptor, VEGF receptor, PsmAr, an extracellular matrix protein, elastin, fibronectin, laminin, α1-antitrypsin, tissue factor protease inhibitor, PDK1, GSK1, Bad, caspase-9, Forkhead, an antigen of Helicobacter pylori, an antigen of Mycobacterium tuberculosis, an antigen of influenza virus, and serum albumin; and
wherein said second single variable domain specifically binds to an antigen selected from the group consisting of, human, protein, animal protein, cytokine, cytokine receptor, ApoE, Apo-SAA, BDNF, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER 3 and HER 4, CD4, human chemokine receptors CXCR4 or CCR5, non-structural protein type 3 (NS3) from the hepatitis C virus, TNF-alpha, IgE, IFN-gamma, MMP-12, CEA, H. pylori, TB, influenza, Hepatitis E, MMP-12, epidermal growth factor receptor (EGFR), ErBb2 receptor on tumor cells, LDL receptor, FGF2 receptor, ErbB2 receptor, transferrin receptor, PDGF receptor, VEGF receptor, PsmAr, an extracellular matrix protein, elastin, fibronectin, laminin, α1-antitrypsin, tissue factor protease inhibitor, PDK1, GSK1, Bad, caspase-9, Forkhead, an antigen of Helicobacter pylori, an antigen of Mycobacterium tuberculosis, and an antigen of influenza virus, and serum albumin.

14. A dual specific ligand comprising a first single variable domain and a second a single variable domain where at least one of the first single variable domain and the second single variable domain comprises a K_{off} for the antigen of 5×10⁻¹ to 1×10⁻⁷ S⁻¹.

15. The ligand of claim 14, wherein variable domain comprises a a K_{off} for the antigen of 1×10⁻² to 1×10⁻⁶S⁻¹.

16. The dual specific ligand of claim 14, wherein said first single variable domain specifically binds to an antigen selected from the group consisting of, human, protein, animal protein, cytokine, cytokine receptor, ApoE, Apo-SAA, BDNF, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER 3 and HER 4, CD4, human chemokine receptors CXCR4 or CCR5, non-structural protein type 3 (NS3) from the hepatitis C virus, TNF-α, IgE, IFN-gamma, MMP-12, CEA, H. pylori, TB, influenza, Hepatitis E, MMP-12, epidermal growth factor receptor (EGFR), ErBb2 receptor on tumor cells, LDL receptor, FGF2 receptor, ErbB2 receptor, transferrin receptor, PDGF receptor, VEGF receptor, PsmAr, an extracellular matrix protein, elastin, fibronectin, laminin, α1-antitrypsin, tissue factor protease inhibitor, PDK1, GSK1, Bad, caspase-9, Forkhead, an antigen of Helicobacter pylori, an antigen of Mycobacterium tuberculosis, an antigen of influenza virus, and serum albumin; and
wherein said second single variable domain specifically binds to an antigen selected from the group consisting of, human, protein, animal protein, cytokine, cytokine receptor, ApoE, Apo-SAA, BDNF, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-β1, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β32, TGF-β3, tumour necrosis factor (TNF), TNF-α, TNF-β, TNF receptor I, TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER 3 and HER 4, CD4, human chemokine receptors CXCR4 or CCR5, non-structural protein type 3 (NS3) from the hepatitis C virus, TNF-alpha, IgE, IFN-gamma, MMP-12, CEA, H. pylori, TB, influenza, Hepatitis E, MMP-12, epidermal growth factor receptor (EGFR), ErBb2 receptor on tumor cells, LDL receptor, FGF2 receptor, ErbB2 receptor, transferrin receptor, PDGF receptor, VEGF receptor, PsmAr, an extracellular matrix protein, elastin, fibronectin, laminin, α1-antitrypsin, tissue factor protease inhibitor, PDK1, GSK1, Bad, caspase-9, Forkhead, an antigen of Helicobacter pylori, an antigen of Mycobacterium tuberculosis, and an antigen of influenza virus, and serum albumin.

17. A dual specific ligand comprising a first single variable domain and a second a single variable domain where at least one of the first single variable domain and the second single variable domain comprises a half life of at least 12 hours in mammalian serum.

18. The ligand of claim 17, wherein the single variable domain comprises a half life of at least 24 hours in mammalian serum.

19. The dual specific ligand of claim 17, wherein said first single variable domain specifically binds to an antigen selected from the group consisting of, human, protein, animal protein, cytokine, cytokine receptor, ApoE, Apo-SAA, BDNF, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8, (72 a.a.), IL-8 (77 a.a.), IL-9, IL-10, IL-11, IL-12, IL-13, IL-15, IL-16, IL-17, IL-18, (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER 3 and HER 4, CD4, human chemokine receptors CXCR4 or CCR5, non-structural protein type 3 (NS3) from the hepatitis C virus, TNF-α, IgE, IFN-gamma, MMP-12, CEA, H. pylori, TB, influenza, Hepatitis E, MMP-12, epidermal growth factor receptor (EGFR), ErBb2 receptor on tumor cells, LDL receptor, FGF2 receptor, ErbB2 receptor, transferrin receptor, PDGF receptor, VEGF receptor, PsmAr, an extracellular matrix protein, elastin, fibronectin, laminin, α1-antitrypsin, tissue factor protease inhibitor, PDK1, GSK1, Bad, caspase-9, Forkhead, an antigen of Helicobacter pylori, an antigen of Mycobacterium tuberculosis, an antigen of influenza virus, and serum albumin; and
wherein said second single variable domain specifically binds to an antigen selected from the group consisting of, human, protein, animal protein, cytokine, cytokine receptor, ApoE, Apo-SAA, BDNF, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-β1, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1αa, MIP-1β, MIP-3α, MIP-3β, MIP-4, myeloid progenitor inhibitor factor-1 (MPIF-1PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER 3 and HER 4, CD4, human chemokine receptors CXCR4 or CCR5, non-structural protein type 3 (NS3) from the hepatitis C virus, TNF-alpha, IgE, IFN-gamma, MMP-12, CEA, H. pylori, TB, influenza, Hepatitis E, MMP-12, epidermal growth factor receptor (EGFR), ErBb2 receptor on tumor cells, LDL receptor, FGF2 receptor, ErbB2 receptor, transferrin receptor, PDGF receptor, VEGF receptor, PsmAr, an extracellular matrix protein, elastin, fibronectin, laminin, α1-antitrypsin, tissue factor protease inhibitor, PDK1, GSK1, Bad, caspase-9, Forkhead, an antigen of Helicobacter pylori, an antigen of Mycobacterium tuberculosis, and an antigen of influenza virus, and serum albumin.


SEQUENCE LISTING (368 sequences)

SEQ ID NO: 1 - protein, 116 aa, Homo Sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSS

SEQ ID NO: 2 - DNA, 348 bp, Homo Sapiens
gaggtgcagctgttggagtctgggggaggcttggtacagcctggggggtccctgcgtctctcctgtgcagcctccggattcacctttagcagctatgccatgagctgggtccgccaggctccagggaagggtctagagtgggtctcagctattagtggtagtggtggtagcacatactacgcagactccgtgaagggccggttcaccatctcccgtgacaattccaagaacacgctgtatctgcaaatgaacagcctgcgtgccgaggacaccgcggtatattactgtgcgaaaagttatggtgcttttgactactggggccagggaaccctggtcaccgtctcgagc

SEQ ID NO: 3 - protein, 108 aa, Homo Sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKR

SEQ ID NO: 4 - DNA, 324 bp, Homo Sapiens
gacatccagatgacccagtctccatcctccctgtctgcatctgtaggagaccgtgtcaccatcacttgccgggcaagtcagagcattagcagctatttaaattggtaccagcagaaaccagggaaagcccctaagctcctgatctatgctgcatccagttggcaaagtggggtcccatcacgtttcagtggcagtggatctgggacagatttcactctcaccatcagcagtctgcaacctgaagattttgctacgtactactgtcaacagagttacagtacccctaatacgttcggccaagggaccaaggtggaaatcaaacgg

SEQ ID NO: 5 - protein, 120 aa, Artificial Sequence
  Artificial antibody domain sequence
EVQLLESGGGLVQPGGSLRLSCAASGFRISDEDMGWVRQAPGKGLEWVSSIYGPSGSTYYADSVKGRFTISRDNSKNTLYLQMNSLRAEDTAVYYCASALEPLSEPLGFWGQGTLVTVSS

SEQ ID NO: 6 - protein, 108 aa, Artificial sequence; identical to SEQ ID NO: 3
  Artificial Antibody Domain Sequence

SEQ ID NO: 7 - protein, 5 aa, Artificial Sequence
  Synthetic Linker Sequence
GGGGS

SEQ ID NO: 8 - protein, 15 aa, Artificial Sequence
  Synthetic Linker Sequence
GGGGSGGGGSGGGGS

SEQ ID NO: 9 - protein, 25 aa, Artificial Sequence
  Synthetic Linker Sequence
GGGGSGGGGSGGGGSGGGGSGGGGS

SEQ ID NO: 10 - protein, 35 aa, Artificial Sequence
  Synthetic Linker Sequence
GGGGSGGGGSGGGGSGGGGSGGGGSGGGGSGGGGS

SEQ ID NO: 11 - protein, 15 aa, Artificial Sequence
  Synthetic Linker Sequence
EPKSGDKTHTCPPCP

SEQ ID NO: 12 - protein, 114 aa, Artificial Sequence
  Artificial Antibody Domain Sequence
WSASTDIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKRC

SEQ ID NO: 13 - DNA, 357 bp, Artificial Sequence
  DNA encoding artificial antibody domain sequence
tggagcgcgtcgacggacatccagatgacccagtctccatcctctctgtctgcatctgtaggagaccgtgtcaccatcacttgccgggcaagtcagagcattgatagttatttacattggtaccagcagaaaccagggaaagcccctaagctcctgatctatagtgcatccgagttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacggtgctaataaggatccggc

SEQ ID NO: 14 - DNA, 39 bp, Artificial Sequence
  Synthetic PCR Primer
tggagcgcgtcgacggacatccagatgacccagtctcca

SEQ ID NO: 15 - DNA, 39 bp, Artificial Sequence
  Synthetic PCR Primer
ttagcagccggatccttattagcaccgtttgatttccac

SEQ ID NO: 16 - protein, 5 aa, Artificial Sequence
  CDR1 Sequence of Synthetic Vk Antibody Domain
XXXLX

SEQ ID NO: 17 - protein, 7 aa, Artificial Sequence
  Artificial Vk CDR2 Sequence
XASXLQS

SEQ ID NO: 18 - protein, 9 aa, Artificial Sequence
  Artificial Vk CDR3 Sequence
QQXXXXPXT

SEQ ID NO: 19 - protein, 5 aa, Artificial Sequence
  Artificial CDR1 Sequence
SSYLN

SEQ ID NO: 20 - protein, 7 aa, Artificial Sequence
  Artificial CDR2 Sequence
RASPLQS

SEQ ID NO: 21 - protein, 9 aa, Artificial Sequence
  Artificial CDR3 Sequence
QQTYSVPPT

SEQ ID NO: 22 - protein, 5 aa, Artificial Sequence
  Artificial CDR1 Sequence
SSYLN

SEQ ID NO: 23 - protein, 7 aa, Artificial Sequence
  Artificial CDR2 Sequence
RASPLQS

SEQ ID NO: 24 - protein, 9 aa, Artificial Sequence
  Artificial CDR3 Sequence
QQTYRIPPT

SEQ ID NO: 25 - protein, 5 aa, Artificial Sequence
  Artificial CDR1 Sequence
FKSLK

SEQ ID NO: 26 - protein, 7 aa, Artificial Sequence
  Artificial CDR2 Sequence
NASYLQS

SEQ ID NO: 27 - protein, 9 aa, Artificial Sequence
  Artificial CDR3 Sequence
QQVVYWPVT

SEQ ID NO: 28 - protein, 5 aa, Artificial Sequence
  Artificial CDR1 Sequence
YYHLK

SEQ ID NO: 29 - protein, 7 aa, Artificial Sequence
  Artificial CDR2 Sequence
KASTLQS

SEQ ID NO: 30 - protein, 9 aa, Artificial Sequence
  Artificial CDR3 Sequence
QQVRKVPRT

SEQ ID NO: 31 - protein, 5 aa, Artificial Sequence
  Artificial CDR1 Sequence
RRYLK

SEQ ID NO: 32 - protein, 7 aa, Artificial Sequence
  Artificial CDR2 Sequence
QASVLQS

SEQ ID NO: 33 - protein, 9 aa, Artificial Sequence
  Artificial CDR3 Sequence
QQGLYPPIT

SEQ ID NO: 34 - protein, 5 aa, Artificial Sequence
  Artificial CDR1 Sequence
YNWLK

SEQ ID NO: 35 - protein, 7 aa, Artificial Sequence
  Artificial CDR2 Sequence
RASSLQS

SEQ ID NO: 36 - protein, 9 aa, Artificial Sequence
  Artificial CDR3 Sequence
QQNVVIPRT

SEQ ID NO: 37 - protein, 5 aa, Artificial Sequence
  Artificial CDR1 Sequence
LWHLR

SEQ ID NO: 38 - protein, 7 aa, Artificial Sequence
  Artificial CDR2 Sequence
HASLLQS

SEQ ID NO: 39 - protein, 9 aa, Artificial Sequence
  Artificial CDR3 Sequence
QQSAVYPKT

SEQ ID NO: 40 - protein, 5 aa, Artificial Sequence
  Artificial CDR1 Sequence
FRYLA

SEQ ID NO: 41 - protein, 7 aa, Artificial Sequence
  Artificial CDR2 Sequence
HASHLQS

SEQ ID NO: 42 - protein, 9 aa, Artificial Sequence
  Artificial CDR3 Sequence
QQRLLYPKT

SEQ ID NO: 43 - protein, 5 aa, Artificial Sequence
  Artificial CDR1 Sequence
FYHLA

SEQ ID NO: 44 - protein, 7 aa, Artificial Sequence
  Artificial CDR2 Sequence
PASKLQS

SEQ ID NO: 45 - protein, 9 aa, Artificial Sequence
  Artificial CDR3 Sequence
QQRARWPRT

SEQ ID NO: 46 - protein, 5 aa, Artificial Sequence
  Artificial CDR1 Sequence
IWHLN

SEQ ID NO: 47 - protein, 7 aa, Artificial Sequence
  Artificial CDR2 Sequence
RASRLQS

SEQ ID NO: 48 - protein, 9 aa, Artificial Sequence
  Artificial CDR3 Sequence
QQVARVPRT

SEQ ID NO: 49 - protein, 5 aa, Artificial Sequence
  Artificial CDR1 Sequence
YRYLR

SEQ ID NO: 50 - protein, 7 aa, Artificial Sequence
  Artificial CDR2 Sequence
KASSLQS

SEQ ID NO: 51 - protein, 9 aa, Artificial Sequence
  Artificial CDR3 Sequence
QQYVGYPRT

SEQ ID NO: 52 - protein, 5 aa, Artificial Sequence
  Artificial CDR1 Sequence
LKYLK

SEQ ID NO: 53 - protein, 7 aa, Artificial Sequence
  Artificial CDR2 Sequence
NASHLQS

SEQ ID NO: 54 - protein, 9 aa, Artificial Sequence
  Artificial CDR3 Sequence
QQTTYYPIT

SEQ ID NO: 55 - protein, 5 aa, Artificial Sequence
  Artificial CDR1 Sequence
LRYLR

SEQ ID NO: 56 - protein, 7 aa, Artificial Sequence
  Artificial CDR2 Sequence
KASWLQS

SEQ ID NO: 57 - protein, 9 aa, Artificial Sequence
  Artificial CDR3 Sequence
QQVLYYPQT

SEQ ID NO: 58 - protein, 5 aa, Artificial Sequence
  Artificial CDR1 Sequence
LRSLK

SEQ ID NO: 59 - protein, 7 aa, Artificial Sequence
  Artificial CDR2 Sequence
AASRLQS

SEQ ID NO: 60 - protein, 9 aa, Artificial Sequence
  Artificial CDR3 Sequence
QQVVYWPAT

SEQ ID NO: 61 - protein, 5 aa, Artificial Sequence
  Artificial CDR1 Sequence
FRHLK

SEQ ID NO: 62 - protein, 7 aa, Artificial Sequence
  Artificial CDR2 Sequence
AASRLQS

SEQ ID NO: 63 - protein, 9 aa, Artificial Sequence
  Artificial CDR3 Sequence
QQVALYPKT

SEQ ID NO: 64 - protein, 5 aa, Artificial Sequence
  Artificial CDR1 Sequence
RKYLR

SEQ ID NO: 65 - protein, 7 aa, Artificial Sequence
  Artificial CDR2 Sequence
TASSLQS

SEQ ID NO: 66 - protein, 9 aa, Artificial Sequence
  Artificial CDR3 Sequence
QQNLFWPRT

SEQ ID NO: 67 - protein, 5 aa, Artificial Sequence
  Artificial CDR1 Sequence
RRYLN

SEQ ID NO: 68 - protein, 7 aa, Artificial Sequence
  Artificial CDR2 Sequence
AASSLQS

SEQ ID NO: 69 - protein, 9 aa, Artificial Sequence
  Artificial CDR3 Sequence
QQMLFYPKT

SEQ ID NO: 70 - protein, 5 aa, Artificial Sequence
  Artificial CDR1 Sequence
IKHLK

SEQ ID NO: 71 - protein, 7 aa, Artificial Sequence
  Artificial CDR2 Sequence
GASRLQS

SEQ ID NO: 72 - protein, 9 aa, Artificial Sequence
  Artificial CDR3 Sequence
QQGARWPQT

SEQ ID NO: 73 - protein, 5 aa, Artificial Sequence
  Artificial CDR1 Sequence
YYHLK

SEQ ID NO: 74 - protein, 7 aa, Artificial Sequence
  Artificial CDR2 Sequence
KASTLQS

SEQ ID NO: 75 - protein, 9 aa, Artificial Sequence
  Artificial CDR3 Sequence
QQVRKVPRT

SEQ ID NO: 76 - protein, 5 aa, Artificial Sequence
  Artificial CDR1 Sequence
YKHLK

SEQ ID NO: 77 - protein, 7 aa, Artificial Sequence
  Artificial CDR2 Sequence
NASHLQS

SEQ ID NO: 78 - protein, 9 aa, Artificial Sequence
  Artificial CDR3 Sequence
QQVGRYPKT

SEQ ID NO: 79 - protein, 5 aa, Artificial Sequence
  Artificial CDR1 Sequence
FKSLX

SEQ ID NO: 80 - protein, 7 aa, Artificial Sequence
  Artificial CDR2 Sequence
NASYLQS

SEQ ID NO: 81 - protein, 9 aa, Artificial Sequence
  Artificial CDR3 Sequence
QQVVYWPVT

SEQ ID NO: 82 - protein, 6 aa, Artificial Sequence
  Consenus CDR1 in VH Library 1
XXYXXX

SEQ ID NO: 83 - protein, 17 aa, Artificial Sequence
  Consenus CDR2 in VH Library 1
XIXXXGXXTXYADSVKG

SEQ ID NO: 84 - protein, 11 aa, Artificial Sequence
  Consensus CDR3 in VH Library 1
XXXXXXXXFDY

SEQ ID NO: 85 - protein, 6 aa, Artificial Sequence
  Artificial CDR1 sequence
WVYQMD

SEQ ID NO: 86 - protein, 17 aa, Artificial Sequence
  Artificial CDR2 sequence
SISAFGAKTLYADSVKG

SEQ ID NO: 87 - protein, 7 aa, Artificial Sequence
  Artificial CDR3 sequence
LSGKFDY

SEQ ID NO: 88 - protein, 6 aa, Artificial Sequence
  Artificial CDR1 sequence
WSYQMT

SEQ ID NO: 89 - protein, 17 aa, Artificial Sequence
  Artificial CDR2 sequence
SISSFGSSTLYADSVKG

SEQ ID NO: 90 - protein, 11 aa, Artificial Sequence
  Artificial CDR3 sequence
GRDHNYSLFDY

SEQ ID NO: 91 - DNA, 27 bp, Artificial Sequence
  Nucleic Acid Sequence for HA Tag
tatccttatgatgttcctgattatgca

SEQ ID NO: 92 - protein, 9 aa, Artificial Sequence
  Amino Acid Sequence for HA Tag
YPYDVPDYA

SEQ ID NO: 93 - protein, 108 aa, Artificial Sequence
  Synthetic Antibody Domain
DIQMTQSPSSLSASVGDRVTITCRASQPIGSFLWWYQQKPGKAPKLLIYYSSYLQSGVPSRFSGSGSGTDFTLTISSLQPEDFATYYCQQYRWHPNTFGQGTKVEIKR

SEQ ID NO: 94 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagcctattgggagttttttatggtggtaccagcagaaaccagggaaagcccctaaactcctgatctattatagttcctatttgcaaagtggggtcccatcacgtttcagtggcagtggatctgggacagatttcactctcaccatcagcagtctgcaacctgaagattttgctacgtactactgtcaacagtatcgttggcatcctaataccttcggccaagggaccaaggtggaaatcaaacgg

SEQ ID NO: 95 - protein, 108 aa, Artificial Sequence
  Synthetic Antibody Domain
DIQMTQSPSSLSASVGDRVTITCRASQSIYSWLNWYQQKPGKAPKLLIYRASHLQSGVPSRFSGSGSGTDFTLTISSLQPEDFATYYCQQIWNMPFTFGQGTKVEIKR

SEQ ID NO: 96 - DNA, 324 bp, Artificial Sequence
  Nucelic Acid Sequence Encoding Synthetic Antibody Domain
gacatccagatgacccagtctccatcctccctgtctgcatctgtaggagaccgtgtcaccatcacttgccgggcaagtcagtcgatttatagttggttaaattggtaccagcagaaaccagggaaagcccctaagctcctgatctatagggcgtcccatttgcaaagtggggtcccatcacgtttcagtggcagtggatctgggacagatttcactctcaccatcagcagtctgcaacctgaagattttgctacgtactactgtcaacagatttggaatatgccttttacgttcggccaagggaccaaggtggaaatcaaacgg

SEQ ID NO: 97 - protein, 108 aa, Artificial Sequence
  Synthetic Antibody Domain
DIQMTQSPSSLSASVGDRVTITCRASQPIGYDLFWYQQKPGKAPKLLIYRGSVLQSGVPSRFSGSGSGTDFTLTISSLQPEDFATYYCQQRWRWPFTFGQGTKVEIKR

SEQ ID NO: 98 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagcctattggttatgatttattttggtaccagcagaaaccagggaaagcccctaagctcctgatctatcggggttccgtgttgcaaagtggggtcccatcacgtttcagtggcagtggatctgggacagatttcactctcaccatcagcagtctgcaacctgaagattttgctacgtactactgtcaacagcggtggcgttggccttttacgttcggccaaggcaccaaggtggaaatcaaacgg

SEQ ID NO: 99 - protein, 107 aa, Artificial Sequence
  Synthetic Antibody Domain
DIQMTQSPSSLSASVGDRVTITCRASLPIGRDLWWYQQKPGKAPKLLIYRGSFLQSGVPSRFSGSGSGTDFTLTISSLQPEDFATYYCQQRWYYPHTFGQGTKVEIK

SEQ ID NO: 100 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtctgcctattggtcgtgatttatggtggtatcagcagaaaccagggaaagcccctaagctcctgatctatcgggggtcctttttgcaaagtggggtcccatcacgtttcagtggcagtggatctgggacagatttcactctcaccatcagcagtctgcaacctgaagattttgctacgtactactgtcaacagaggtggtattatcctcatacgttcggccaagggaccaaggtggaaatcaaacgg

SEQ ID NO: 101 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcatttttatgaatttattgtggtaccagcagaaaccagggaaagcccctaagctcctgatctataatgcatccgtgttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgc

SEQ ID NO: 102 - DNA, 323 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcatttggacgaagttacattggtaccagcagaaaccagggaaagcccctaagctcctgatctatatggcatccagtttgcaaagtggggtcccatcacgtttcagtggcagtggatctgggacagatttcactctcaccatcagcagtctgcaacctgaagattttgctacgtactactgtcaacagtggtttagtaatcctagtacgttcggccaagggaccaaggtggaaatcaaacg

SEQ ID NO: 103 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgagcattatttatggtggtaccagcagaaaccagggaaagcccctaagctcctgatctatgctgcatcctatttgcaaagtggggtcccatcacgtttcagtggcagtggatctgggacagatttcactctcaccatcagcagtctgcaacctgaagattttgctacgtactactgtcaacagagtttggcgtgtcctcctacgttcggccaagggaccaaggtggaaatcaaacgg

SEQ ID NO: 104 - DNA, 324 bp, Artificial Sequence
  Nucleic Acid Sequence  Encoding Synthetic Antibody
gacatccagatgacccagtctccatcctccctgtctgcatctgtaggagaccgtgtcaccatcacttgccgggcaagtcagagcatttatggtcatttattgtggtaccagcagaaaccagggaaagcccctaagctcctgatctatgctgcatccagtttgcaaagtggggtcccatcacgtttcagtggcagtggatctgggacagatttcactctcaccatcagcagtctgcaacctgaagattttgctacgtactactgtcaacagcctttggtgcggccttttacgttcggccaagggaccaaggtggaaatcaaacgg

SEQ ID NO: 105 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgctaagttgttatattggtaccagcagaaaccagggaaagcccctaagctcctgatctatgatgcatcctctttgcaaagtggggtcccatcacgtttcagtggcagtggatctgggacagatttcactctcaccatcagcagtctgcaacctgaagattttgctacgtactactgtcaacagtggtgggggtatcctggtacgttcggccaagggaccaaggtggaaatcaaacgg

SEQ ID NO: 106 - DNA, 324 bp, Artificial Sequence
  Nucliec Acid Sequence Encoding Synthetic Antibody Domain
gacatccagatgacccagtctccatcctccctgtctgcatctgtaggagaccgtgtcaccatcacttgccgggcaagtcagagcatttttcctgctttactttggtaccagcagaaaccagggaaagcccctaagctcctgatctatcatgcatccagtttgcaaagtggggtcccatcacgtttcagtggcagtggatctgggacagatttcactctcaccatcagcagtctgcaacctgaagatattgctacgtactactgtcaacaggttgtgtggcgtccttttacgttcggccaagggaccaaggtggaaatcaaacgg

SEQ ID NO: 107 - DNA, 224 bp, Artificial Sequence
  Nucleic Acid Sequence Encoding Synthetic Antibody Domain
attggtaccagcagaaaccagggaaagcccctaagctcctgatctatcaggcatccattttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 108 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcatttttatgaatttattgtggtaccagcagaaaccagggaaagcccctaagctcctgatctataatgcatccgtgttgcaaagtggggtcccatcacgtttcagtggcagtggatctgggacaggtttcactctcaccatcagcagtctgcaacctgaagattttgctacgtactactgtcaacaggttgtgtggcgtccttttacgttcggccaagggaccaaggtggaaatcaaacgg

SEQ ID NO: 109 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattttgaattctttacattggtaccagcagaaaccagggaaagcccctaagctcctgatctatcatgcatccactttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 110 - DNA, 324 bp, Artificial Sequence; identical to SEQ ID NO: 109
  Nucleic Acid Sequence Encoding Synthetic Antibody Domain

SEQ ID NO: 111 - DNA, 325 bp, Artificial Sequence
  Nucleic Acid Encoding Synthetic Antibody Domain
gacatccagatgacccagtctccatcctccctgtctgcatctgtaggagaccgtgtcaccatcacttgccgggcaagtcagagcattgataattatttacattggtaccagcagaaaccagggaaagcccctaagctcctgatctattctgcatcccatttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacggv

SEQ ID NO: 112 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattaatgagtatttacattggtaccagcagaaaccagggaaagcccctaagctcctgatctattctgcatccgtgttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 113 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattaattatgctttacattggtaccagcagaaaccagggaaagcccctaagctcctgatctatcaggcatccattttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 114 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgatagttttttacattggtaccagcagaaaccagggaaagcccctaagctcctgatctatagtgcatccgagttgcaaagtggggtcccatcacgtttcagtggcagtggatctgggacagatttcactctcaccatcagcagtctgcatcctgaagattttgctacgtactactgtcaacaggttgtgtggcgtccttttacgttcggccaagggaccaaggtggaaatcaaacgg

SEQ ID NO: 115 - DNA, 324 bp, Artificial Sequence
  Nucleic Acid Sequence Encoding Synthetic Antibody Domain
gacatccagatgacccagtctccatcctctctgtctgcatctgtaggagaccgtgtcaccatcacttgccgggcaagtcagagcattgatagttatttacattggtaccagcagaaaccagggaaagcccctaagctcctgatctatagtgcatccgagttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 116 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgatcagtatttacattggtaccagcagaaaccagggaaagcccctaagctcctgatctatggtgcatccaatttgcaaagtgaggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 117 - DNA, 324 bp, Artificial Sequence; identical to SEQ ID NO: 114
  Nucleic Acid Sequence Encoding Synthetic Antibody Domain

SEQ ID NO: 118 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgattcttatttacattggtaccagcagaaaccagggaaagcccctaagctcctgatctatagtgcatccctgttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 119 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgatcagtatttacattggtaccagcagaaaccagggaaagcccctaagctcctgatctattctgcatcccttttgcaaagtggggtcccatcacgtttcagtggcagtggatctgggacagatttcactctcaccatcagcagtctgcaacctgaagattttgctacatactactgtcaacaggttgtgtggcgtccttttacgttcggccaagggaccaaggtggaaatcaaacgg

SEQ ID NO: 120 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcaaagcattgatgagtttttacattggtaccagcagaaaccagggaaagcccctaagctcctgatctattgtgcatcccagttgcaaagtggggtcccatcacgtttcagtggcagtggatctgggacagatttcactctcaccatcagcagtctacatcctgaagattttgctacgtactactgtcaacaggttgtgtggcgtccttttacgttcggccaagggaccaaggtggaaatcaaacgg

SEQ ID NO: 121 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgatgcgtatttacattggtaccagcagaaaccagggaaagcccctaagctcctgatctattctgcatccctgttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 122 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgataggtatttacattggtaccagcagaaaccagggaaagcccctaagctcctgatctatagtgcatccgtgttgcaaagtggggtcccatcacgtttcagtggcagtggatctgggacagatttcaccctcaccatcagcagtctgcagcctgaagattttgctacgtactactgtcaacaggttgtgtggcgtccttttacgttcggccaagggaccaaggtggaaatcaaacgg

SEQ ID NO: 123 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgataagtatttacattggtaccagcagaaaccagggaaagcccctaagctcctgatctatagtgcatcctcgttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 124 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgatcattatttacattggtaccagcagaaaccagggaaagcccctaagctcctgatctatagtgcatccgttttgcaaagtggggtcccatcacgtttcagtggcagtggatctgggacagatttcactctcaccatcagcagtctgcaacctgaagattttgcaacgtactactgtcaacaggttgtgtggcgtccttttacgttcggccaagggaccaaggtggaaatcaaacgg

SEQ ID NO: 125 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgatgagtttttacattggtaccagcagaaaccagggaaagcccctaagctcctgatctatagtgcatccattttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 126 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattcagactgcgttactgtggtaccagcagaaaccagggaaagcccctaagctcctgatctataatgcatccagtttgcaaagtggggtcccatcacgtttcagtggcagtggatctgggacagatttcactctcaccatcagcagtctgcaacctgaagattttgctacatactactgtcaacaggttgtgtggcgtccttttacgttcggccaagggaccaaggtggaaatcaaacgg

SEQ ID NO: 127 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgatcagtatttacattggtaccagcagaaaccagggaaagcccctaagctcctgatctatggtgcatccaatttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 128 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgataattatttacattggtaccagcagaaaccagggaaagcccctaagctcctgatctatagtgcatcccagttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 129 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgataattttttacattggtaccagcagaaaccagggaaagcccctaagctcctgatctatagtgcatccgagttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 130 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgatgagtatttacattggtaccagcagaaaccagggaaaccccctaagctcctgatctattctgcatccagtttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 131 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgatcattttttacattggtaccagcagaaaccagggaaagcccctaagctcctgatctatagtgcatccgagttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 132 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgataattatttacattggtaccagcagaaaccagggaaagcccctaagctcctgatctattcggcatccatgttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 133 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgatgagtatttacattggtaccagcagaaaccagggaaagcccccaagctcctgatctattctgcatccattttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 134 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgatgagtttttacattggtaccagcagaaaccagggaaagcccctaagctcctgatctattcggcatccgctttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 135 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgatgagtatttacattggtaccagcagaaaccagggaaagcccctaagctcctgatctattctgcatccattttgcaaagtggggtcccatcacgtttcagtggcagtggatctgggacagatttcactctcaccatcagcagtctgcaccctgaagattttgctacgtactactgtcaacaggttgtgtggcgtccttttacgttcggccaagggaccaaggtggaaatcaaacgg

SEQ ID NO: 136 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgataattatttacattggtaccagcagaaaccagggaaagcccctaagctcctgatctatgctgcatccagtttgcaaagtggggtcccatcacgtttcagtggcagtggatctgggacagatttcactctcaccatcagcagtctgcaacctgatgattttgctacgtactactgtcaacaggttgtgtggcgtccttttgcgttcggccaagggaccaaggtggaaatcaaacgg

SEQ ID NO: 137 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagagcattgatagttatttacattggtaccagcagaaaccagggaaagcccctaagctcctgatctatagtgcatcaaatttagaaacaggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 138 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcaggtgatttgggatgcgttagattggtaccagcagaaaccagggaaagcccctaagctcctgatctatagtgcgtcccgtttgcaaagtggggtcccatcacgtttcagtggcagtggatctgggacagatttcactctcaccatcagcagtctgcatcctgaagattttgctacgtactactgtcaacagtatgctgtgtttcctgtgacgttcggccaagggaccaaggtggaaatcaaacgg

SEQ ID NO: 139 - DNA, 324 bp, Artificial Sequence
  Nucleic Acid Sequence Encoding Synthetic Antibody Domain
gacatccagatgacccagtctccatcctccctgtctgcatctgtaggagaccgtgtcaccatcacttgccgggcaagtcagactatttatgatgcgttaagttggtaccagcagaaaccagggaaagcccctaagctcctgatctatggtggttccaggttgcaaagtggggtcccatcacgtttcagtggcggtggatctgggacagatttcactctcaccatcagcagtctgcaacctgaagattttgctacgtactactgtcaacagtataagactaagcctttgacgttcggccaagggaccaaggtggaaatcaaacgg

SEQ ID NO: 140 - DNA, 324 bp, Artificial Sequence
  Nucleic Acid Sequence Encoding Synthetic Antibody Domain
gacatccagatgacccagtccccatcctccctgtctgcatctgtaggagaccgtgtcaccatcacttgccgggcaagtcagactatttatgatgcgttaagttggtaccagcagaaaccagggaaagcccctaagctcctgatctatggtggttccaggttgcaaagtggggtcccatcacgtttcagtggtagtggatctgggacagatttcactctcaccatcagcagtctgcaacccgaagattttgctacgtactactgtcaacagtatgctcgttatcctcttacgttcggccaagggaccaaggtggaaatcaaacgg

SEQ ID NO: 141 - protein, 108 aa, Artificial Sequence
  Synthetic Antibody Domain
DIQMTQSPSSLSASVGDRVTITCRASQDIEEWLMWYQQKPGKAPKLLIYNSSTLQSGVPSRFSGSGSGTDFTLTISSLQPEDYATYYCQQPLSRPFTFGQGTKVEIKR

SEQ ID NO: 142 - protein, 108 aa, Artificial Sequence
  Synthetic Antibody Domain
DIQMTQSPSSLSASVGDRVTITCRASQHIDDWLFWYQQKPGKAPKLLIYRASFLQSGVPSRFSGSGSGTDFTLTISSLQPEDFATYYCQQPLSRPFTFGQGTKVEIKR

SEQ ID NO: 143 - protein, 108 aa, Artificial Sequence
  Synthetic Antibody Domain
DIQMTQSPSSLSASVGDRVTITCRASQFIEDWLFWYQQKPGKAPKLLIYQASKLQSGVPSRFSGSGSGTDFTLTISSLQPEDFATYYCQQPLSRPFTFGQGTKVEIKR

SEQ ID NO: 144 - protein, 108 aa, Artificial Sequence
  Synthetic Antibody Domain
DIQMTQSPSSLSASVGDRVTITCRASQPIDSWLMWYQQKPGKAPKLLIYQASRLQSGVPSRFSGSGSGTDFTLTISSLQPEDFATYYCQQPLSRPFTFGQGTKVEIKR

SEQ ID NO: 145 - protein, 108 aa, Artificial Sequence
  Synthetic Antibody Domain
DIQMTQSPSSLSASVGDRVTITCRASQHIDDWLFWYQQKPGKAPKLLIYRASFLQSGVPPRFSGSGSGTDFTLTISSLQPEDFATYYCQQPLSRPFTFGQGTKVEIKR

SEQ ID NO: 146 - protein, 108 aa, Artificial Sequence
  Synthetic Antibody Domain
DIQMTQSPSSLSASVGDRVTITCRASQNIDDHLMWYQQKPGKAPKLLIYSSSILQSGVPPRFSGSGSGTDFTLTISSLQPEDFATYYCQQPLSRPFTFGQGTKVEIKR

SEQ ID NO: 147 - protein, 108 aa, Artificial Sequence
  Synthetic Antibody Domain
DIQMTQSPSSLSASVGDRVTITCRASQDIDHALLWYQQKPGKAPRLLIYNGSMLQSGVPSRFSGSGSGTDFTLTISSLQPEDFATYYCQQVLRRPFTFGQGTKVEIKR

SEQ ID NO: 148 - protein, 108 aa, Artificial Sequence
  Synthetic Antibody Domain
DIQMTQSPSSLSASVGDRVTITCRASQHIGDWLLWYQQKPGKAPMLLIYQSSRLQSGVPSRFSGSGSGTDFILTISSLQPEDFATYYCQQPLSRPFTFGQGTKVEIKR

SEQ ID NO: 149 - protein, 108 aa, Artificial Sequence
  Synthetic Antibody Domain
DIQMTQSPSSLSASVGDRVTITCRASQHIDSYLMWYQQKPGKAPKLLIYNTSVLQSGVPSRFSGSGSGTDFTLTISSLQPEDFATYYCQQPLSRPFTFGQGTKVEIKR

SEQ ID NO: 150 - protein, 108 aa, Artificial Sequence
  Synthetic Antibody Domain
DIQMTQSPSSLSASVGDRVTITCRASQWIDDHLFWYQQKPGKAPKLLIYNTSTLQSGVPSRFSGSGSGTDFILTISSLQPEDFATYYCQQPLSRPFTFGQGTKVEIKR

SEQ ID NO: 151 - protein, 108 aa, Artificial Sequence
  Synthetic Antibody Domain
DIQMTQSPSSLSASVGDRVTITCRASQFIDEHLMWYQQKPGKAPKLLIYRSSELQSGVPSRFSGSGSGTDFTLTISSLQPEDFATYYCQQPLSRPFTFGQGTKVEIKR

SEQ ID NO: 152 - protein, 108 aa, Artificial Sequence
  Synthetic Antibody Domain
DIQMTQSPSSLSASVGDRVTITCRASQWINNWLLWYQQKPGKAPKLLIYESSNLQSGVPSRFSGSGSGTDFTLTISSLQPEDFATYYCQQPLSRPFTFGQGTKVEIKR

SEQ ID NO: 153 - protein, 107 aa, Artificial Sequence
  Synthetic Antibody Domain
DIQMTQSPSSLSASVGDRVTITCRASQLIDDHFWYQQKPGKAPTLLIYNSSVLQSGVPSRFSGSGSGTDFTLTISSLQPEDFATYYCQQPLSRPFTFGQGTKVEIKR

SEQ ID NO: 154 - protein, 108 aa, Artificial Sequence
  Synthetic Antibody Domain
DIQMTQSPSSLSASVGDRVTITCRASQDIDQWLMWYQQKPGKAPKLLIYQSSMLQSGVPSRFSGSGSGTDFTLTISSLQPEDFATYYCQQPLSRPFTFGQGTKVEIKR

SEQ ID NO: 155 - protein, 108 aa, Artificial Sequence
  Synthetic Antibody Domain
DIQMTQSPSSLSASVGDRVTITCQASQDIDNWLLWYQQKPGKAPKLLIYQASNLQSGVPSRFSGSGSGTDFTLTISSLQPEDFATYYCQQPLSRPFTFGQGTKVEIKR

SEQ ID NO: 156 - protein, 108 aa, Artificial Sequence
  Synthetic Antibody Domain
DIQMTQSPSSLSASVGDRVTITCRASQPIDSWLMWYQQKPGKAPKLLIYQASRLQSGVPSRFSGSGSGTDFTLTISSLQPEDFATYYCQQPLSGPFTFGQGTKVEIKR

SEQ ID NO: 157 - protein, 108 aa, Artificial Sequence
  Synthetic Antibody Domain
DIQMTQSPSSLSASVGDRVTITCRASQYIDYGLMWYQQKPGKAPKLLIYRTSELQSGVPSRFSGSGSGTDFTLTISSLQPEDFATYYCQQPLSRPFTFGQGTKVEIKR

SEQ ID NO: 158 - protein, 108 aa, Artificial Sequence
  Synthetic Antibody Domain
DIQMTQSPSSLSASVGDRVTITCRASQWIDSFLMWYQQKPGKAPKLLIYNGSVLQSGVPSRFSGSGSGTDFTLTISSLQPEDFATYYCQQPLSRPFTFGQGTKVEIKR

SEQ ID NO: 159 - protein, 108 aa, Artificial Sequence
  Synthetic Antibody Domain
DIQMTQSPSSLSASVGDRVTITCRASQDIGPWLMWYQQKPGKAPKLLIYQGSRLQSGVPLRFSGSGSGTDFTLTISSLQPEDFATYYCQQPLSRPFTFGQGTKVEIRR

SEQ ID NO: 160 - protein, 108 aa, Artificial Sequence
  Synthetic Antibody Domain
DIQMTQSPSSLSASVGDRVTITCRASQHIDSWLLWYQQKPGKAPKLLIYNGSVLQSGVPSRFSGSGSGTDFTLTISSLQPEDFATYYCQQPLSGPFTFGQGTKVEIKR

SEQ ID NO: 161 - protein, 108 aa, Artificial Sequence
  Synthetic Antibody Domain
DIQMTQSPSSLSASVGDRVTITCRASQHIDTHLFWYQQKPGKAPKLLIYNTSTLQSGVPSRFSGSGSGTDFTLTISSLQPEDFATYYCQQPLSRPFTFGQGTKVEIKR

SEQ ID NO: 162 - protein, 108 aa, Artificial Sequence
  Synthetic Antibody Domain
DIQMTQSPSSLSASVGDRVTITCRASQFIDTHLMWYQQKPGKAPRLLIYNTSTLQSGVPSRFSGSGSGTDFTLTISSLQPEDFATYYCQQPLSRPFTFGQGTKVEIKR

SEQ ID NO: 163 - protein, 108 aa, Artificial sequence
  Synthetic antibody domain sequence
DIQMTQSPSSLSASVGDRVTITCRASQDIDDWLLWYQQKPGKAPKLLIYQGSRLQSGVPSRFSGSGSGTDFTLTISSLQPEDFATYYCQQPLSRPFTFGQGTKVEIKR

SEQ ID NO: 164 - protein, 108 aa, Artificial Sequence
  Synthetic Antibody Domain
DIQMTQSPSSLSASVGDRVTITCRASQWIDDTLMWYQQKPGKAPKLLIYRSSMLQSGVPSRFSGSGSGTDFTLTISSLQPEDFATYYCQQPLSRPFTFGQGTKVEIKR

SEQ ID NO: 165 - protein, 108 aa, Artificial Sequence
  Synthetic Antiobdy Domain
DIQMTQSPSSLSASVGDRVTITCRASQYIDSHLMWYQQKPGKAPKLLIYDTSRLQSGVPSRFSGSGSGTDFTLTISSLQPEDFATYYCQQPLSRPFTFGQGTKVEIKR

SEQ ID NO: 166 - protein, 108 aa, Artificial Sequence
  Synthetic Antibody Domain
DIQMTQSPSSLSASVGDRVTITCRASQHIDQHLFWYQQKPGKAPKLLIYNSSSLQSGVPSRFSGSGSGTDFTLTISSLQPEDFATYYCQQPLSRPFTFGQGTKVEIKR

SEQ ID NO: 167 - protein, 108 aa, Artificial Sequence
  Synthetic Antibody Domain
DIQMTQSPSSLSASVGDRVTITCRASQHIERWLLWYQQKPGKAPKLLIYNSSKLQSGVPSRFSGSGSGTDFTLTISSLQPEDFATYYCQQPLSRPFTFGQGTKVEIKR

SEQ ID NO: 168 - protein, 108 aa, Artificial Sequence
  Synthetic Antibody Domain
DIQMTQSPSSLSASVGDRVTISCRASQHIERWLLWYQQKPGKAPKLLIYNSSKLQSGVPSRFSGSGSGTDFTLTISSLQPEDFATYYCQQPLSRPFTFGQGTKVEIKR

SEQ ID NO: 169 - protein, 108 aa, Artificial Sequence
  Synthetic Antibody Domain
DIQMTQSPSSLSASVGDRVTITCRASQDIGSWLMWYQQKSGKAPKLLIYNGSALQSGVPSRFSGSGSGTDFTLTISSLQPEDFATYYCQQPLSRPFTFGQGTKVEIKR

SEQ ID NO: 170 - protein, 108 aa, Artificial Sequence
  Synthetic Antibody Domain
DIQMTQSPSSLSASVGDRVTITCRASQHIDKWLMWYQQKPGKAPKLLIYQASKLQSGVPSRFSGSGSGTDFTLTISSLQPEDFATYYCQQPLSRPFTFGQGTKVEIKR

SEQ ID NO: 171 - protein, 108 aa, Artificial Sequence
  Synthetic Antibody Domain
DIQMTQSPSSLSASVGDRVTITCRASQDIEEWLMWYQQKPGKAPKLLIYNSSTLQSGVPSRFSGSGSGTDFTLTISSLQPEDFATYYCQQPLSRPFTFGQGTKVEIKR

SEQ ID NO: 172 - protein, 100 aa, Artificial Sequence
  Synthetic Antibody Domain
DIQMTQSPSSLSASVGDRVTITCRASQYIDYGLMWYQQKPGKAPKLLIYRTSELQSGVPSRFSGSGSGTDFTLTISSLQPEDFATYYCQQPLSRPFTFGQ

SEQ ID NO: 173 - protein, 108 aa, Artificial Sequence
  Synthetic Antibody Domain
DIQMTQSPSSLSASVGDRVTITCRASQNIDIHLMWYQQKPGKAPKLLIYQSSNLQSGVPSPFSGSGSGTDFTLTISSLQPEDFATYYCQQPLSRPFTFGQGTKVEIKR

SEQ ID NO: 174 - protein, 108 aa, Artificial Sequence
  Synthetic Antibody Domain
DIQMTQSPSSLSASVGDRVTITCRASQDIGPWLLWYQQKPGKAPKLLIYQSSELQSGVPSRFSGSGSGTDFTLTISSLQPEDLATYYCQQPLSRPFTFGQGTKVEIKR

SEQ ID NO: 175 - protein, 108 aa, Artificial Sequence
  Synthetic Antibody Domain
DIQMTQSPSSLSASVGDRVTITCRASQEIGVWLMWYQQKPGKAPKLLIYEGSRLQSGVPSRFSGSGSGTDFTLTISSLQPEDFATYYCQQPLSRPFVFGQGTKVEIKR

SEQ ID NO: 176 - protein, 108 aa, Artificial Sequence
  Synthetic Antibody Domain
DIQMTQSPSSLSASVGDRVTITCRASQSIGKWLMWYQQKPGKAPKLLIYQSSLLQSGVPSRFSGSGSGTDFTLTISSLQPEDFATYYCQQPLSRPFTFGQGTKVEIKR

SEQ ID NO: 177 - protein, 108 aa, Artificial Sequence
  Synthetic Antibody Domain
DIQMTQSPSSLSASVGDRVTITCRASQDIDTWLFWYQQKPGKAPKLLIYNGSRLQSGVPSRFSGSGSGTDFTLTISGLQPEDFATYYCQQPLSRPFTFGQGTKVEIKR

SEQ ID NO: 178 - protein, 108 aa, Artificial Sequence; identical to SEQ ID NO: 144
  Synthetic Antibody Domain

SEQ ID NO: 179 - protein, 108 aa, Artificial Sequence
  Synthetic Antibody Domain
DIQMTQSPSSLSASVGDRVTITCRASQDIEGWLLWYQQKPGKAPKLLIYNSSTLQSGVPSRFSGSGSGTDFTLTISSLQPEDFATYYCQQPLSRPFTFGQGTKVEIKR

SEQ ID NO: 180 - protein, 108 aa, Artificial Sequence; identical to SEQ ID NO: 142
  Synthetic Antibody Domain

SEQ ID NO: 181 - protein, 108 aa, Artificial Sequence; identical to SEQ ID NO: 177
  Synthetic Antibody Domain

SEQ ID NO: 182 - protein, 108 aa, Artificial Sequence
  Synthetic Antibody Domain
DIQMTQSPSSLSASVGDRVTITCRASQPIEEWLLWYQQKPGKAPKLLIYNGSHLQSGVPSRFSGSGSGTDFTLTISSLQPEDFATYYCQQPLSRPFTFGQGTKVEIKR

SEQ ID NO: 183 - protein, 108 aa, Artificial Sequence; identical to SEQ ID NO: 170
  Synthetic Antibody Domain

SEQ ID NO: 184 - protein, 108 aa, Artificial Sequence; identical to SEQ ID NO: 141
  Synthetic Antibody Domain

SEQ ID NO: 185 - protein, 108 aa, Artificial Sequence
  Synthetic Antibody Domain
DIQMTQSPSSLSASVGDRVTITCRASQPIDYGLMWYQQKPGKAPKLLIYRSSQLQSGVPSRFSGSGSGTDFTLTISSLQPEDFATYYCQQPLSRPFTFGQGTKVEIKR

SEQ ID NO: 186 - protein, 108 aa, Artificial Sequence
  Synthetic Antibody Domain
DIQMTQSPSSLSASVGDRVTITCRASQEIGSWLMWYQQKPGKAPKLLIYQSSKLQSGVPSRFSGSGSGTDFTLTISSLQPEDFATYYCQQPLSRPFTFGQGTKVEIKR

SEQ ID NO: 187 - protein, 108 aa, Artificial Sequence
  Synthetic Antibody Domain
DIQMTQSPSSLSASVGDRVTITCRASQPIDSWLLWYQQKPGKAPKLLIYNASSLQSGVPSRFSGSGSGTDFTLTISSLQPEDFATYYCQQPLSRPFTFGQGTKVEIKR

SEQ ID NO: 188 - protein, 108 aa, Artificial Sequence
  Synthetic Antibody Domain
DIQMTQSPSSLSASVGDRVTITCRASQDIGPWLMWYQQKPGKAPKLLIYQASALQSGVPSRFSGSGSGTDFTLTISSLQPEDFATYYCQQPLSRPFTFGQGTKVEIKR

SEQ ID NO: 189 - protein, 108 aa, Artificial Sequence
  Synthetic Antibody Domain
DIQMTQSPSSLSASVGDRVTITCRASQNIHEWLMWYQQKPGKAPKLLIYQGSRLQSGVPSRFSGSGSGTDFTLTISSLQPEDFATYYCQQPLSRPFTFGQGTKVEIKR

SEQ ID NO: 190 - protein, 108 aa, Artificial Sequence
  Synthetic Antibody Domain
DIQMTQSPSSLSASVGDRVTITCRASQDIGPWLMWYQQKPGKAPKLLIYQASALQSGVPSRFSGSGSGTDFTLTISSLQPEDSATYYCQQPLSRPFTFGQGTKVEIKR

SEQ ID NO: 191 - protein, 108 aa, Artificial Sequence
  Synthetic Antibody Domain
DIQMTQSPSSLSASVGDRVTITCRASQSVKEFLWWYQQKPGKAPKLLIYMASNLQSGVPSRFSGSGSGTDFTLTISSLQPEDFATYYCQQKFKLPRTFGQGTKVEIKR

SEQ ID NO: 192 - protein, 108 aa, Artificial Sequence
  Synthetic Antibody Domain
DIQMTQSPSSLSASVGDRVTITCRASQWIGPELSWYQQKPGKAPKLLIYHGSILQSGVPSRFSGSGSGTDFTLTISSLQPEDFATYYCQQRMYRPATFGQGTKVEIKR

SEQ ID NO: 193 - protein, 108 aa, Artificial Sequence
  Synthetic Antibody Domain
DIQMTQSPSSLSASVGDRVTITCRASQWIGRELKWYQQKPGKAPRLLIYHGSVLQSGVPSRFSGSGSGTDFTLTISSLQPEDFATYYCQQDFFVPDTFGQGTKVEIKR

SEQ ID NO: 194 - protein, 108 aa, Artificial Sequence
  Synthetic Antibody Domain
DIQMTQSPSSLSASVGDRVTITCRASQDIANDLMWYQQKPGKAPKLLIYRNSRLQGGVPSRFSGSGSGTDFTLTISSLQPEDFATYYCQQLVHRPYTIGQGTKVEIKR

SEQ ID NO: 195 - protein, 108 aa, Artificial Sequence
  Synthetic Antibody Domain
DIQMTQSPSSLSASVGDRVTITCRASQFIGPHLTWYQQKPGKAPKLLIYHSSLLQSGVPSRFSGSGSGTDFTLTISSLQPEDFATYYCQQYMYYPSTFGQGTKVKIKR

SEQ ID NO: 196 - protein, 108 aa, Artificial Sequence
  Synthetic Antibody Domain
DIQMTQSPSSLSASVGDRVTITCRASQWIGPELSWYQQKPGKAPKLLIYHTSILQSGVPSRFSGSGSGTDFTLTISSLQPEDFATYYCQQYMFQPRTFGQGTKVEIRR

SEQ ID NO: 197 - protein, 108 aa, Artificial Sequence
  Synthetic Antibody Domain
DIQMIQSPSSLSASVGDRVTITCRASQFIGNELSWYQQKPGKAPKLLIYHASSLQSGVPSRFSGSGSGTDFTLTISSLQPEDFATYYCQQVLGYPYTFGQGTKVEIKR

SEQ ID NO: 198 - protein, 108 aa, Artificial Sequence
  Synthetic Antibody Domain
DIQMTQSPSSLSASVGDRVTITCRASQWIGPELSWYQQKPGKAPKLLIYHGSILQSGVPSRFSGSGSGTDFTLTISSLQPEDFATYYCQQVLYSPLTFGQGTKVEIKR

SEQ ID NO: 199 - protein, 108 aa, Artificial Sequence
  Synthetic Antibody Domain
DIQMTQSPSSLSASVGDRVTITCRASQWIGNELKWYQQKPGKAPKLLIYMSSLLQSGVPSRFSGSGSGTDFTLTISSLQPEDLATYYCQQTLLLPFTFGQGTKVEIKR

SEQ ID NO: 200 - protein, 108 aa, Artificial Sequence
  Synthetic Antibody Domain
DIQMTQSPSSLSASVGDRVTITCRASQWIGPELSWYQQKPGKAPKLLIYHGSILQSGVPSRFSGSGSGTDFTLTISSLQPEDFATYYCQQRLYYPGTFGQGTKVEIKR

SEQ ID NO: 201 - protein, 108 aa, Artificial Sequence
  Synthetic Antibody Domain
DIQMTQSPSSLSASVGDRVTITCRASQSIGRELSWYQQKPGKAPMLLIYHSSNLQSGVPSRFSGSGSGTDFTLTISSLQPEDFATYYCQQGMYWPYTFGQGTKVEIKR

SEQ ID NO: 202 - protein, 108 aa, Artificial Sequence
  Synthetic Antibody Domain
DIQMTQSPSSLSASVGDRVTITCRASQWIKPALHWYQQKPGKAPKLLIYHGSILQSGVPSRFSGSGSGTDFTLTISSLQPEDFATYYCQQTLFMPYTFGQGTKVEIKR

SEQ ID NO: 203 - protein, 108 aa, Artificial Sequence
  Synthetic Antibody Domain
DIQMTQSPSSLSASVGDRVTITCRASQSISTALLWYQQKPGKAPKLLIYNGSMLPNGVPSRFSGSGSGTDFTLTISSLQPEDFATYYCQQTWDTPMTFGQGTKVEIKR

SEQ ID NO: 204 - protein, 108 aa, Artificial Sequence
  Synthetic Antibody Domain
DIQMTQSPSSLSASVGDRVTITCRASQWIGHDLSWYQQKPGKAPKLLIYHSSSLQSGVPSRFSGSGSGTDFTLTISSLQPEDVATYYCQQLMGYPFTFGQGTKVEIKR

SEQ ID NO: 205 - protein, 108 aa, Artificial Sequence
  Synthetic Antibody Domain
DIQMTQSPSSLSASVGDRVTITCRASQDIGGLLVWYQQKPGKAPKLLIYRSSYLQSGVPSRFSGSGSGTDFTLTISSLQPEDFATYYCQQTWGIPHTFGQGTKVEIKR

SEQ ID NO: 206 - protein, 108 aa, Artificial Sequence
  Synthetic Antibody Domain
DIQMTQSPSSLSASVGDRVTITCRASQKIFNGLSWYQQKPGKAPKLLIYHSSTLQSGVPSRFSGSGSGTDFTLTISSLQPEDFATYYCQQVLLYPYTFGQGTKVEIKR

SEQ ID NO: 207 - protein, 108 aa, Artificial Sequence
  Synthetic Antibody Domain
DIQMTQSPSSLSASVGDRVTITCRASQSIGTNLSWYQQKPGKAPRLLIYRTSMLQSGVPSRFSGSGSGTDFTLTISSLQPEDFATYYCQQQFFWPHTFGQGTKVEIKR

SEQ ID NO: 208 - protein, 118 aa, Artificial Sequence
  Synthetic Antibody Domain
EVQLLESGGGLVQPGGSLRLSCAASGFTFRLYDMVWVRQAPGKGLEWVSYISSGGSGTYYADSVKGRFTISRDNSKNTLYLQMNSLRAEDTAVYYCAKAGGRASFDYWGQGTLVTVSS

SEQ ID NO: 209 - protein, 116 aa, Artificial Sequence
  Synthetic Antibody Domain
EVQLLESGGGLVQPGGSLRLSCAASGFTFHLYDMMWVRQAPGKGLEWVSFIGGDGLNTYYADSVKGRFTISRDNSKNTLYLQMNSLRAEDTAVYYCAKAGTQFDYWGQGTLVTVSS

SEQ ID NO: 210 - protein, 119 aa, Artificial Sequence
  Synthetic Antibody Domain
EVQLLESGGGLVQPGGSLRLSCAASGFTFNKYPMMWVRQAPGKGLEWVSEISPSGQDTYYADSVKGRFTISRDNSKNTLYLQMNSLRAEDTAVYYCAKNPQILSNFDYWGQGTLVTVSS

SEQ ID NO: 211 - protein, 124 aa, Artificial Sequence
  Synthetic Antibody Domain
EVQLLESGGGLVQPGGSLRLSCAASGFTFQWYPMWWVRQAPGKGLEWVSLIEGQGDRTYYADSVKGRFTISRDNSKNTLYLQMNSLRAEDTAVYYCAKAGDRTAGSRGNSFDYWGQGTLVTVSS

SEQ ID NO: 212 - protein, 121 aa, Artificial Sequence
  Synthetic Antibody Domain
EVQLLESGGGLVQPGGSLRLSCAASGFTFKAYEMGWVRQAPGKGLEWVSGISPNGGWTYYADSVKGRFTISRDNSKNTLYLQMNSLRAEDTAVYYCAKESISPTPLGFDYWGQGTLVTVSS

SEQ ID NO: 213 - protein, 116 aa, Artificial Sequence
  Synthetic Antibody Domain
EVQLLESGGGLVQPGGSLRLSCAASGFTFTGYEMGWVRQAPGKGLEWVSYISRGGRWTYYADSVKGRFTISRDNSKNTLYLQMNSLRAEDTAVYYCAKSDTMFDYWGQGTLVTVSS

SEQ ID NO: 214 - protein, 116 aa, Artificial Sequence
  Synthetic Antibody Domain
EVQLLESGGGLVQPGGSLRLSCAASGFTFSAYEMGWVRQAPGKGLEWVSFISGGGRWTYYADSVKGRFTISRDNSKNTLYLQMNSLRAEDTAVYYCAKYSEDFDYWGQGTLVTVSS

SEQ ID NO: 215 - protein, 116 aa, Artificial Sequence
  Synthetic Antibody Domain
EVQLLESGGGLVQPGGSLRLSCAASGFTFGAYPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 216 - protein, 123 aa, Artificial Sequence
  Synthetic Antibody Domain
EVQLLESGGGLVQPGGSLRLSCAASGFTFQFYKMGWVRQAPGKGLEWVSSISSVGDATYYADSVKGRFTISRDNSKNTLYLQMNSLRAEDTAVYYCAKMGGGPPTYVVYFDYWGQGTLVTVSS

SEQ ID NO: 217 - protein, 123 aa, Artificial Sequence
  Synthetic Antibody Domain
EVQLLESGGGLVQPGGSLRLSCAASGFTFGEYGMYWVRQAPGKGLEWVSSISERGRLTYYADSVKGRFTISRDNSKNTLYLQMNNLRAEDTAVYYCAKSALSSEGFSRSFDYWGQGTLVTVSS

SEQ ID NO: 218 - protein, 123 aa, Artificial Sequence
  Synthetic Antibody Domain
EVQLLESGGGLVQPGGSLRLSCAASGFTFSDYAMYWVRQAPGKGLEWVSSITARGFITYYADSVKGRFTISRDNSKNTLYLQMNSLRAEDTAVYYCAKSGFPHKSGSNYFDYWGQGTLVTVSS

SEQ ID NO: 219 - protein, 240 aa, Artificial Sequence
  Synthetic Antibody Sequence, VH and VL joined by Gly4Ser Linker
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSGGGGSGGGGSGGGGSTDIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKR

SEQ ID NO: 220 - DNA, 720 bp, Artificial Sequence
  Nucleotide Sequence Encoding Synthetic Antibody Sequence, VH and VL joined by Gly4Ser Linker
gaggtgcagctgttggagtctgggggaggcttggtacagcctggggggtccctgcgtctctcctgtgcagcctccggattcacctttagcagctatgccatgagctgggtccgccaggctccagggaagggtctagagtgggtctcagctattagtggtagtggtggtagcacatactacgcagactccgtgaagggccggttcaccatctcccgtgacaattccaagaacacgctgtatctgcaaatgaacagcctgcgtgccgaggacaccgcggtatattactgtgcgaaaagttatggtgcttttgactactggggccagggaaccctggtcaccgtctcgagcggtggaggcggttcaggcggaggtggcagcggcggtggcgggtcgacggacatccagatgacccagtctccatcctccctgtctgcatctgtaggagaccgtgtcaccatcacttgccgggcaagtcagagcattagcagctatttaaattggtaccagcagaaaccagggaaagcccctaagctcctgatctatgctgcatccagttggcaaagtggggtcccatcacgtttcagtggcagtggatctgggacagatttcactctcaccatcagcagtctgcaacctgaagattttgctacgtactactgtcaacagagttacagtacccctaatacgttcggccaagggaccaaggtggaaatcaaacgg

SEQ ID NO: 221 - DNA, 359 bp, Artificial Sequence
  Phage Vector Expression Cassette Nucleotide Sequences
caggaaacagctatgaccatgattacgccaagcttgcatgcaaattctatttcaaggagacagtcataatgaaatacctattgcctacggcagccgctggattgttattactcgcggcccagccggccatggccgaggtgtttgactactggggccagggaaccctggtcaccgtctcgagcggtggaggcggttcaggcggaggtggcagcggcggtggcgggtcgacggacatccagatgacccaggcggccgcagaacaaaaactccatcatcatcaccatcacggggccgcaatctcagaagaggatctgaatggggccgcatagactgttgaaagttgtttagcaaaacctcat

SEQ ID NO: 222 - protein, 96 aa, Artificial Sequence
  Expression Cassette Amino Acid Sequences
MKYLLPTAAAGLLLLAAQPAMAEVFDYWGQGTLVTVSSGGGGSGGGGSGGGGSTDIQMTQAAAEQKLHHHHHHGAAISEEDLNGAATVESCLAKPH

SEQ ID NO: 223 - protein, 116 aa, Artificial Sequence
  VH Sequence of Clone K8
EVQLLESGGGLVQPGGSLRLSCAASGFTFSSYAMSWVRQAPGKGLEWVSHISPYGANTRYADSVKGRFTISRDNSKNTLYLQMNSLRAEDTAVYYCAKGLRAFDYWGQGTLVTVSS

SEQ ID NO: 224 - protein, 116 aa, Artificial Sequence
  VH Sequence of Clone VH2
EVQLLESGGGLVQPGGSLRLSCAASGFTFSSYAMSWVRQAPGKGLEWVSDIGATGSKTGYADPVKGRFTISRDNSKNTLYLQMNSLRAEDTAVYYCAKKVLTFDYWGQGTLVTVSS

SEQ ID NO: 225 - protein, 115 aa, Artificial Sequence
  VH Sequence of Clone VH4
EVQLLESGGGLVQPGGSLRLSCAASGFTFSSYAMSWVRQAPGKGLEWVSRINGPGATGYADSVKGRFTISRDNSKNTLYLQINSLRAEDTAVYYCAKHGAPFDYWGQGTLVTVSS

SEQ ID NO: 226 - protein, 116 aa, Artificial Sequence
  VH Sequence of Clone VHC11
EVQLLESGGGLVQPGGSLRLSCAASGFTFSSYAMNWVRQAPGKGLEWVSSIPASGLHTRYADSVKGRFTISRDNSKNTLYLQMNSLRAEDTAVYYCAKPGLGFDYWGQGTLVTVSS

SEQ ID NO: 227 - protein, 115 aa, Artificial Sequence
  VH Sequence of Clone VHA10sd
EVQLLESGGGLVQPGGSLRLSCAASGFTFSSYAMSWVRQAPGKGLEWVSDIERTGYTRYADSVKGRFTISRDNSKNTLYLQMNSLRAEDTAVYYCAKKVLVFDYWGQGTLVTVSS

SEQ ID NO: 228 - protein, 116 aa, Artificial Sequence
  VH Sequence of clone VHA1sd
EVQLLESGGGLVQPGGSLRLSCAASGFTFSSYAMSWVRQAPGKGLEWVSEISANGSKTQYADSVKGRLTISRDNSKNTLYLQMNSLRAEDTAVYYCAKKVLQFDYWGQGTLVTVSS

SEQ ID NO: 229 - protein, 115 aa, Artificial Sequence
  VH Sequence of Clone VHA5sd
EVQLLESGGGLVQPGGSLRLSCAASGFTFSSYAMSWVRQAPGKGLEWVSTIPANGVTRYADSVKGRFTISRDNSKNTLYLQMNSLRAEDTAVYYCAKSLLQFDYWGQGTLVTVSS

SEQ ID NO: 230 - protein, 116 aa, Artificial Sequence
  VH Sequence of Clone VHC5sd
EVQLLESGGGLVQPGGSLRLSCAASGFTFSSYAMSWVRQAPGKGLEWVSDIAATGSATSYADSVKGRFTISRDNSKNTLYLQMNSLRAEDTAVYYCAKKILKFDYWGQGTLVTVSS

SEQ ID NO: 231 - protein, 116 aa, Artificial Sequence
  VH Sequence of Clone VHC11sd
EVQLLESGGGLVQPGGSLRLSCAASGSTFSSYAMSWVRQAPGKGLEWVSTISSVGQSTRYADSVKGRFTISRDNSKNTLYLQMNSLRAEDTAVYYCAKNLMSFDYWGQGTLVTVSS

SEQ ID NO: 232 - protein, 108 aa, Artificial Sequence
  Vk Sequence of Clone K8
DIQMTQSPSSLSASVGDRVTITCRASQSISSYLNWYQQKPGKAPKLLIYRASHLQSGVPSRFSGSGSGTDFTLTISSLQPEDFATYYCQQPWRSPGTFGQGTKVEIKR

SEQ ID NO: 233 - protein, 108 aa, Artificial Sequence
  Vk Sequence of Clone E5sd
DIQMTQSPSSLSASVGDRVTITCRASQSVSSYLNWYQQKPGKAPKLLIYLASRLQSGVPSRFSGSGSGTDFTLTISSLQPEDFATYYCQQNWWLPPTFGQGTKVEIKR

SEQ ID NO: 234 - protein, 107 aa, Artificial Sequence
  Vk Sequence of Clone C3
DIQMTQSPSSLSASVGDRVTITCRASQSISSYLNWYQQKPGKAPKLLIYASLLQSGVPSRFSGSGSGTDFTLTISSLQPEDFATYYCQQRVYDPLTFGQGTKVEIKR

SEQ ID NO: 235 - protein, 120 aa, Artificial Sequence
  Dummy VH for Library
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAXXXXFDYWGQGTLVTVSS

SEQ ID NO: 236 - DNA, 360 bp, Artificial Sequence
  Nucleotide Sequence for Dummy VH for Library
gaggtgcagctgttggagtctgggggaggcttggtacagcctggggggtccctgcgtctctcctgtgcagcctccggattcacctttagcagctatgccatgagctgggtccgccaggctccagggaagggtctagagtgggtctcagctattagtggtagtggtggtagcacatactacgcagactccgtgaagggccggttcaccatctcccgtgacaattccaagaacacgctgtatctgcaaatgaacagcctgcgtgccgaggacaccgcggtatattactgtgcgaaaagttatggtgctnnknnknnknnktttgactactggggccagggaaccctggtcaccgtctcgagc

SEQ ID NO: 237 - protein, 108 aa, Artificial Sequence
  Sequence of Anti-Murine Serum Albumin Domain Antibody
DIQMTQSPSSLSASVGDRVTITCRASQSIIKHLKWYQQKPGKAPKLLIYGASRLQSGVPSRFSGSGSGTDFTLTISSLQPEDFATYYCQQGARWPQTFGQGTKVEIKR

SEQ ID NO: 238 - DNA, 324 bp, Artificial Sequence
  Nucleotide Sequence Encoding Anti-Murine Serum Albumin Domain Anitbody
gacatccagatgacccagtctccatcctccctgtctgcatctgtaggagaccgtgtcaccatcacttgccgggcaagtcagagcattattaagcatttaaagtggtaccagcagaaaccagggaaagcccctaagctcctgatctatggtgcatcccggttgcaaagtggggtcccatcacgtttcagtggcagtggatctgggacagatttcactctcaccatcagcagtctgcaacctgaagattttgctacgtactactgtcaacagggggctcggtggcctcagacgttcggccaagggaccaaggtggaaatcaaacgg

SEQ ID NO: 239 - protein, 108 aa, Artificial Sequence
  Sequence of Anti-Murine Serum Albumin Domain Antibody
DIQMTQSPSSLSASVGDRVTITCRASQSIYYHLKWYQQKPGKAPKLLIYKASTLQSGVPSRFSGSGSGTDFTLTISSLQPEDFATYYCQQVRKVPRTFGQGTKVEIKR

SEQ ID NO: 240 - DNA, 324 bp, Artificial Sequence
  Nucleotide Sequence Encoding Anti-Murine Serum Albumin Domain Antibody
gacatccagatgacccagtctccatcctccctgtctgcatctgtaggagaccgtgtcaccatcacttgccgggcaagtcagagcatttattatcatttaaagtggtaccagcagaaaccagggaaagcccctaagctcctgatctataaggcatccacgttgcaaagtggggtcccatcacgtttcagtggcagtggatctgggacagatttcactctcaccatcagcagtctgcaacctgaagattttgctacgtactactgtcaacaggttcggaaggtgcctcggacgttcggccaagggaccaaggtggaaatcaaacgg

SEQ ID NO: 241 - DNA, 324 bp, Artificial Sequence; identical to SEQ ID NO: 101
  Nucleotide Sequence of Anti-Murine Serum Albumin Domain Antibody

SEQ ID NO: 242 - DNA, 323 bp, Artificial Sequence; identical to SEQ ID NO: 102
  Nucleotide Sequence of Anti-Murine Serum Albumin Domain Antibody

SEQ ID NO: 243 - DNA, 324 bp, Artificial Sequence; identical to SEQ ID NO: 103
  Nucleotide Sequence of Anti-Murine Serum Albumin Domain Antibody

SEQ ID NO: 244 - DNA, 324 bp, Artificial Sequence; identical to SEQ ID NO: 104
  Nucleotide Sequence of Anti-Murine Serum Albumin Domain Antibody

SEQ ID NO: 245 - DNA, 324 bp, Artificial Sequence; identical to SEQ ID NO: 105
  Nucleotide Sequence of Anti-Murine Serum Albumin Domain Antibody

SEQ ID NO: 246 - DNA, 324 bp, Artificial Sequence; identical to SEQ ID NO: 106
  Nucleotide Sequence of Anti-Murine Serum Albumin Domain Antibody

SEQ ID NO: 247 - DNA, 324 bp, Artificial Sequence
  Nucleotide Sequence of Anti-Murine Serum Albumin Domain Antibody
gacatccagatgacccagtctccatcctccctgtctgcatctgtaggagaccgtgtcaccatcacttgccgggcaagtcagagcattgataatgcgttacattggtaccagcagaaaccagggaaagcccctaagctcctgatctatcaggcatccattttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 248 - DNA, 324 bp, Artificial Sequence; identical to SEQ ID NO: 108
  Nucleotide Sequence of Anti-Murine Serum Albumin Domain Antibody

SEQ ID NO: 249 - DNA, 324 bp, Artificial Sequence; identical to SEQ ID NO: 109
  Nucleotide Sequence of Anti-Murine Serum Albumin Domain Antibody

SEQ ID NO: 250 - DNA, 324 bp, Artificial Sequence; identical to SEQ ID NO: 109
  Nucleotide Sequence of Anti-Murine Serum Albumin Domain Antibody

SEQ ID NO: 251 - DNA, 324 bp, Artificial Sequence
  Nucleotide Sequence of Anti-Murine Serum Albumin Domain Antibody
gacatccagatgacccagtctccatcctccctgtctgcatctgtaggagaccgtgtcaccatcacttgccgggcaagtcagagcattgataattatttacattggtaccagcagaaaccagggaaagcccctaagctcctgatctattctgcatcccatttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 252 - DNA, 324 bp, Artificial Sequence; identical to SEQ ID NO: 112
  Nucleotide Sequence of Anti-Murine Serum Albumin Domain Antibody

SEQ ID NO: 253 - DNA, 324 bp, Artificial Sequence; identical to SEQ ID NO: 113
  Nucleotide Sequence of Anti-Murine Serum Albumin Domain Antibody

SEQ ID NO: 254 - DNA, 324 bp, Artificial Sequence; identical to SEQ ID NO: 114
  Nucleotide Sequence of Anti-Murine Serum Albumin Domain Antibody

SEQ ID NO: 255 - DNA, 324 bp, Artificial Sequence; identical to SEQ ID NO: 116
  Nucleotide Sequence of Anti-Murine Serum Albumin Domain Antibody

SEQ ID NO: 256 - DNA, 324 bp, Artificial Sequence; identical to SEQ ID NO: 114
  Nucleotide Sequence of Anti-Murine Serum Albumin Domain Antibody

SEQ ID NO: 257 - DNA, 324 bp, Artificial Sequence; identical to SEQ ID NO: 118
  Nucleotide Sequence of Anti-Murine Serum Albumin Domain Antibody

SEQ ID NO: 258 - DNA, 324 bp, Artificial Sequence; identical to SEQ ID NO: 119
  Nucleotide Sequence of Anti-Murine Serum Albumin Domain Antibody

SEQ ID NO: 259 - DNA, 324 bp, Artificial Sequence; identical to SEQ ID NO: 120
  Nucleotide Sequence of Anti-Murine Serum Albumin Domain Antibody

SEQ ID NO: 260 - DNA, 324 bp, Artificial Sequence; identical to SEQ ID NO: 121
  Nucleotide Sequence of Anti-Murine Serum Albumin Domain Antibody

SEQ ID NO: 261 - DNA, 324 bp, Artificial Sequence; identical to SEQ ID NO: 122
  Nucleotide Sequence of Anti-Murine Serum Albumin Domain Antibody

SEQ ID NO: 262 - DNA, 324 bp, Artificial Sequence; identical to SEQ ID NO: 123
  Nucleotide Sequence of Anti-Murine Serum Albumin Domain Antibody

SEQ ID NO: 263 - DNA, 324 bp, Artificial Sequence; identical to SEQ ID NO: 124
  Nucleotide Sequence of Anti-Murine Serum Albumin Domain Antibody

SEQ ID NO: 264 - DNA, 324 bp, Artificial Sequence; identical to SEQ ID NO: 125
  Nucleotide Sequence of Anti-Murine Serum Albumin Domain Antibody

SEQ ID NO: 265 - DNA, 324 bp, Artificial Sequence; identical to SEQ ID NO: 126
  Nucleotide Sequence of Anti-Murine Serum Albumin Domain Antibody

SEQ ID NO: 266 - DNA, 324 bp, Artificial Sequence; identical to SEQ ID NO: 127
  Nucleotide Sequence of Anti-Murine Serum Albumin Domain Antibody

SEQ ID NO: 267 - DNA, 324 bp, Artificial Sequence; identical to SEQ ID NO: 128
  Nucleotide Sequence of Anti-Murine Serum Albumin Domain Antibody

SEQ ID NO: 268 - DNA, 324 bp, Artificial Sequence; identical to SEQ ID NO: 129
  Nucleotide Sequence of Anti-Murine Serum Albumin Domain Antibody

SEQ ID NO: 269 - DNA, 324 bp, Artificial Sequence; identical to SEQ ID NO: 130
  Nucleotide Sequence of Anti-Murine Serum Albumin Domain Antibody

SEQ ID NO: 270 - DNA, 324 bp, Artificial Sequence; identical to SEQ ID NO: 131
  Nucleotide Sequence of Anti-Murine Serum Albumin Domain Antibody

SEQ ID NO: 271 - DNA, 324 bp, Artificial Sequence; identical to SEQ ID NO: 132
  Nucleotide Sequence of Anti-Murine Serum Albumin Domain Antibody

SEQ ID NO: 272 - DNA, 324 bp, Artificial Sequence; identical to SEQ ID NO: 133
  Nucleotide Sequence of Anti-Murine Serum Albumin Domain Antibody

SEQ ID NO: 273 - DNA, 324 bp, Artificial Sequence; identical to SEQ ID NO: 134
  Nucleotide Sequence of Anti-Murine Serum Albumin Domain Antibody

SEQ ID NO: 274 - DNA, 324 bp, Artificial Sequence; identical to SEQ ID NO: 135
  Nucleotide Sequence of Anti-Murine Serum Albumin Domain Antibody

SEQ ID NO: 275 - DNA, 324 bp, Artificial Sequence; identical to SEQ ID NO: 136
  Nucleotide Sequence of Anti-Murine Serum Albumin Domain Antibody

SEQ ID NO: 276 - DNA, 324 bp, Artificial Sequence; identical to SEQ ID NO: 137
  Nucleotide Sequence of Anti-Murine Serum Albumin Domain Antibody

SEQ ID NO: 277 - DNA, 324 bp, Artificial Sequence; identical to SEQ ID NO: 138
  Nucleotide Sequence of Anti-Murine Serum Albumin Domain Antibody

SEQ ID NO: 278 - DNA, 324 bp, Artificial Sequence; identical to SEQ ID NO: 139
  Nucleotide Sequence of Anti-Murine Serum Albumin Domain Antibody

SEQ ID NO: 279 - DNA, 324 bp, Artificial Sequence; identical to SEQ ID NO: 140
  Nucleotide Sequence of Anti-Murine Serum Albumin Domain Antibody

SEQ ID NO: 280 - protein, 120 aa, Artificial Sequence; identical to SEQ ID NO: 5
  Amino Acid Sequence of Domain Antibody

SEQ ID NO: 281 - DNA, 360 bp, Artificial Sequence
  Nucleotide sequence of domain antibody
gaggtgcagctgttggagtctgggggaggcttggtacagcctggggggtccctgcgtctctcctgtgcagcctccggatttaggattagcgatgaggatatgggctgggtccgccaggctccagggaagggtctagagtgggtatcaagcatttatggccctagcggtagcacatactacgcagactccgtgaagggccggttcaccatctcccgtgacaattccaagaacacgctgtatctgcaaatgaacagcctgcgtgccgaggacaccgcggtatattattgcgcgagtgctttggagccgctttcggagcccctgggcttttggggtcagggaaccctggtcaccgtctcgagc

SEQ ID NO: 282 - protein, 116 aa, Artificial Sequence
  Amino Acid Sequence of Domain Antibody
EVQLLESGGGLVQPGGSLRLSCAASGFTFDLYNMFWVRQAPGKGLEWVSFISQTGRLTWYADSVKGRFTISRDNSKNTLYLQMNSLRAEDTAVYYCAKTLEDFDYWGQGTLVTVSS

SEQ ID NO: 283 - DNA, 348 bp, Artificial Sequence
  Nucleotide Sequence of Domain Antibody
gaggtgcagctgttggagtctgggggaggcttggtacagcctggggggtccctgcgtctctcctgtgcagcctccggattcacctttgatctttataatatgttttgggtccgccaggctccagggaagggtctagagtgggtctcatttattagtcagactggtaggcttacatggtacgcagactccgtgaagggccggttcaccatctcccgcgacaattccaagaacacgctgtatctgcaaatgaacagcctgcgtgccgaggacaccgcggtatattactgtgcgaaaacgctggaggattttgactactggggccagggaaccctggtcaccgtctcgagc

SEQ ID NO: 284 - protein, 108 aa, Artificial Sequence; identical to SEQ ID NO: 191
  Amino Acid Sequence of Domain Antibody

SEQ ID NO: 285 - DNA, 324 bp, Artificial Sequence
  Nucleotide Sequence of Domain Antibody
gacatccagatgacccagtctccatcctccctgtctgcatctgtaggagaccgtgtcaccatcacttgccgggcaagtcagagcgttaaggagtttttatggtggtaccagcagaaaccagggaaagcccctaagctcctgatctatatggcatccaatttgcaaagtggggtcccatcacgtttcagtggcagtggatctgggacagatttcactctcaccatcagcagtctgcaacctgaagattttgctacgtactactgtcaacagaagtttaagctgcctcgtacgttcggccaagggaccaaggtggaaatcaaacgg

SEQ ID NO: 286 - protein, 120 aa, Artificial Sequence
  Amino Acid Sequence of Domain Antibody
EVQLLESGGGLVQPGGSLRLSCAASGFTFEWYWMGWVRQAPGKGLEWVSAISGSGGSTYYADSVKGRFTISRDNSKNTLYLQMNSLRAEDTAVYYCAKVKLGGGPNFDYWGQGTLVTVSS

SEQ ID NO: 287 - DNA, 362 bp, Artificial Sequence
  Nucleotide Sequence of Domain Antibody
gaggtgcagctgttggagtctgggggaggcttggtacagcctggggggtccctgcgtctctcctgtgcagcctccggattcacctttgagtggtattggatgggttgggtccgccaggctccagggaagggtctagagtgggtctcagctattagtggtagtggtggtagcacatactacgcagactccgtgaagggccggttcaccatctcccgcgacaattccaagaacacgctgtatctgcaaatgaacagcctgcgtgccgaggacaccgcggtatattactgtgcgaaagttaagttggggggggggcctaattttgactactggggccagggaaccctggtcaccgtctcgagcgc

SEQ ID NO: 288 - protein, 108 aa, Artificial Sequence
  Amino Acid Sequence of Domain Antibody
DIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKR

SEQ ID NO: 289 - DNA, 324 bp, Artificial Sequence
  Nucleotide Sequence of Domain Antibody
gacatccagatgacccagtctccatcctctctgtctgcatctgtaggagaccgtgtcaccatcacttgccgggcaagtcagagcattgatagttatttacattggtaccagcagaaaccagggaaagcccctaagctcctgatctatagtgcatccgagttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgc

SEQ ID NO: 290 - protein, 108 aa, Artificial Sequence
  Amino Acid Sequence of Domain Antibody
DIQMTQSPSSLSASVGDRVTITCRASQSIFMNLLWYQQKPGKAPKLLIYNASVLQSGVPSRFSGSGSGTDFTLTISSLQPEDFATYYCQQVVWRPFTFGQGTKVEIKR

SEQ ID NO: 291 - DNA, 324 bp, Artificial Sequence
  Nucleotide Sequence of Domain Antibody
gacatccagatgacccagtctccatcctccctgtctgcatctgtaggagaccgtgtcaccatcacttgccgggcaagtcagagcatttttatgaatttattgtggtaccagcagaaaccagggaaagcccctaagctcctgatctataatgcatccgtgttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 292 - protein, 108 aa, Artificial Sequence
  Amino Acid Sequence of Domain Antibody
DIQMTQSPSSLSASVGDRVTITCRASQSIYDALEWYQQKPGKAPKLLIYTASRLQSGVPSRFSGSGSGTDFTLTISSLQPEDFATYYCQQVMQRPVTFGQGTKVEIKR

SEQ ID NO: 293 - DNA, 324 bp, Artificial Sequence
  Nucleotide Sequence of Domain Antibody
gacatccagatgacccagtctccatcctccctgtctgcatctgtaggagaccgtgtcaccatcacttgccgggcaagtcagagcatttatgatgcgttagagtggtaccagcagaaaccagggaaagcccctaagctcctgatctatactgcatcccggttgcaaagtggggtcccatcacgtttcagtggcagtggatctgggacagatttcactctcaccatcagcagtctgcaacctgaagattttgctacgtactactgtcaacaggttatgcagcgtcctgttacgttcggccaagggaccaaggtggaaatcaaacgg

SEQ ID NO: 294 - protein, 33 aa, Artificial sequence
  Domain antibody amino acid sequence
DIQMTQSPSSLSASVGDRVTITCRASQSIYDAL

SEQ ID NO: 295 - protein, 74 aa, Artificial sequence
  Domain antibody amino acid sequence
WYQQKPGKAPKLLIYTASRLQSGVPSRFSGSGSGTDFTLTISSLQPEDFATYHCQQVMQRPVTFGQGTKVEIKR

SEQ ID NO: 296 - DNA, 324 bp, Artificial Sequence
  Nucleotide Sequence of Domain Antibody
gacatccagatgacccagtctccatcctccctgtctgcatctgtaggagaccgtgtcaccatcacttgccgggcaagtcagagcatttatgatgctttacagtggtaccagcagaaaccagggaaagcccctaagctcctgatctatactgcatcccggttgcaaagtggggtcccatcacgtttcagtggcagtggatctgggacagatttcactctcaccatcagcagtctgcaacctgaagattttgctacgtaccactgtcaacaggttatgcagcgtcctgttacgttcggccaagggaccaaggtggaaatcaaacgg

SEQ ID NO: 297 - protein, 108 aa, Artificial Sequence; identical to SEQ ID NO: 191
  Amino Acid Sequence of Domain Antibody

SEQ ID NO: 298 - DNA, 324 bp, Artificial Sequence; identical to SEQ ID NO: 285
  Nucleotide Sequence of Domain Antibody

SEQ ID NO: 299 - protein, 108 aa, Artificial Sequence
  Amino Acid Sequence of Domain Antibody
DIQMTQSPSSLSASVGDRVTITCRASQSIWTKLHWYQQKPGKAPKLLIYMASSLQSGVPSRFSGSGSGTDFTLTISSLQPEDFATYYCQQWFSNPSTFGQGTKVEIKR

SEQ ID NO: 300 - DNA, 324 bp, Artificial Sequence
  Nucleotide Sequence of Domain Antibody
gacatccagatgacccagtctccatcctccctgtctgcatctgtaggagaccgtgtcaccatcacttgccgggcaagtcagagcatttggacgaagttacattggtaccagcagaaaccagggaaagcccctaagctcctgatctatatggcatccagtttgcaaagtggggtcccatcacgtttcagtggcagtggatctgggacagatttcactctcaccatcagcagtctgcaacctgaagattttgctacgtactactgtcaacagtggtttagtaatcctagtacgttcggccaagggaccaaggtggaaatcaaacgc

SEQ ID NO: 301 - protein, 29 aa, Artificial Sequence
  Amino Acid Sequence of Domain Antibody
DIQMTQSPSSLSASVGDRVTITCRASQSI

SEQ ID NO: 302 - protein, 78 aa, Artificial sequence
  Domain antibody amino acid sequence
PILCWYQQKPGKAPKLLIYAASSLQSGVPSRFSGSGSGTDFTLTISSLQPEDFATYYCQQIQHIPVTFGQGTKVEIKR

SEQ ID NO: 303 - DNA, 324 bp, Artificial Sequence
  Nucleotide Sequence of Domain Antibody
gacatccagatgacccagtctccatcctccctgtctgcatctgtaggagaccgtgtcaccatcacttgccgggcaagtcagagcatttagccgattttatgttggtaccagcagaaaccagggaaagcccctaagctcctgatctatgctgcatccagtttgcaaagtggggtcccatcacgtttcagtggcagtggatctgggacagatttcactctcaccatcagcagtctgcaacctgaagattttgctacgtactactgtcaacagattcagcatattcctgtgacgttcggccaagggaccaaggtggaaatcaaacgg

SEQ ID NO: 304 - protein, 30 aa, Artificial Sequence
  Amino Acid Sequence of Domain Antibody
DIQMTQSPSSLSASVGDRVTITCRASQSIG

SEQ ID NO: 305 - protein, 77 aa, Artificial sequence
  Domain antibody amino acid sequence
DLHWYQQKPGKAPKLLIYTASLLQSGVPSRFSGSGSGTDFTLTISSLQPEDFATYYCQQQSAFPNTLGQGTKVEIKR

SEQ ID NO: 306 - DNA, 324 bp, Artificial Sequence
  Nucleotide Sequence of Domain Antibody
gacatccagatgacccagtctccatcctccctgtctgcatctgtaggagaccgtgtcaccatcacttgccgggcaagtcagagcattgggtaggatttacattggtaccagcagaaaccagggaaagcccctaagctcctgatctatacggcatcccttttgcaaagtggggtcccatcacgtttcagtggcagtggatctgggacagatttcactctcaccatcagcagtctgcaacctgaagattttgctacgtactactgtcaacagcagagtgcttttcctaatacgctcggccaagggaccaaggtggaaatcaaacgg

SEQ ID NO: 307 - protein, 49 aa, Artificial Sequence
  Amino Acid Sequence of Domain Antibody
DIQMTQSPSSLSASVGDRVTITCRASQSITKNLLWYQQKPGKAPKLLIY

SEQ ID NO: 308 - protein, 58 aa, Artificial sequence
  Domain antibody amino acid sequence
ASSLQSGVPSRFSGSGSGTDFTLTISSLQPEDFATYYCQQLRHKPPTFGQGTKVEIKR

SEQ ID NO: 309 - DNA, 324 bp, Artificial Sequence
  Nucleotide Sequence of Domain Antibody
gacatccagatgacccagtctccatcctccctgtctgcatccgtaggagaccgtgtcaccatcacttgccgggcaagtcagagcataacgaagaatttactttggtaccagcagaaaccagggaaagcccctaagctcctgatctattaggcatcctctttgcaaagtggggtcccatcacgtttcagtggcagtggatctgggacagatttcactctcaccatcagcagtctgcaacctgaagattttgctacgtactactgtcaacagcttcgtcataagcctccgacgttcggccaagggaccaaggtggaaatcaaacgg

SEQ ID NO: 310 - protein, 29 aa, Artificial Sequence
  Amino Acid Sequence of Domain Antibody
DIQMTQSPSSLSASVGDRVTITCRASQSI

SEQ ID NO: 311 - protein, 78 aa, Artificial sequence
  Domain antibody amino acid sequence
KSLRWYQQKPGKAPKLLIYHASDLQSGVPSRFSGSGSGTDFTLTISSLQPEDFATYYCQQMVNSPVTFGQGTKVEIKR

SEQ ID NO: 312 - DNA, 324 bp, Artificial Sequence
  Nucleotide Sequence of Domain Antibody
gacatccagatgacccagtctccatcctccctgtctgcatctgtaggagaccgtgtcaccatcacttgccgggcaagtcagagcatttagaagtctttaaggtggtaccagcagaaaccagggaaagcccctaagctcctgatctatcatgcatccgatttgcaaagtggggtcccatcacgtttcagtggcagtggatctgggacagatttcactctcaccatcagcagtctgcaacctgaagattttgctacgtactactgtcaacagatggttaatagtcctgttacgttcggccaagggaccaaggtggaaatcaaacgg

SEQ ID NO: 313 - protein, 29 aa, Artificial Sequence
  Amino Acid Sequence of Domain Antibody
DIQMTQSPSSLSASVGDRVTITCRASQSI

SEQ ID NO: 314 - protein, 78 aa, Artificial sequence
  Domain antibody amino acid sequence
TALHWYQQKPGKAPKLLIYSASSLQSGVPSRFSGSGSGTDFTLTISSLQPEDFATYYCQQSSFLPFTFGQGTKVEIKR

SEQ ID NO: 315 - DNA, 324 bp, Artificial Sequence
  Nucleotide Sequence of Domain Antibody
gacatccagatgacccagtctccatcctccctgtctgcatctgtaggagaccgtgtcaccatcacttgccgggcaagtcagagcatttagacggcgttacattggtaccagcagaaaccagggaaagcccctaagctcctgatctattctgcatccagtttgcaaagtggggtcccatcacgtttcagtggcagtggatctgggacagatttcactctcaccatcagcagtctgcaacctgaagattttgctacgtactactgtcaacagtcgagttttttgccttttacgttcggccaagggaccaaggtggaaatcaaacgg

SEQ ID NO: 316 - protein, 108 aa, Artificial Sequence
  Amino Acid Sequence of Domain Antibody
DIQMTQSPSSLSASVGDRVTITCRASQSIGPNLEWYQQKPGKAPKLLIYAASSLQSGVPSRFSGSGSGTDFTLTISSLQPEDFATYYCQQQMGRPRTFGQGTKVEIKR

SEQ ID NO: 317 - DNA, 324 bp, Artificial Sequence
  Nucleotide Sequence of Domain Antibody
gacatccagatgacccagtctccatcctccctgtctgcatctgtaggagaccgtgtcaccatcacttgccgggcaagtcagagcattgggccgaatttagagtggtaccagcagaaaccagggaaagcccctaagctcctgatctatgctgcatccagtttgcaaagtggggtcccatcacgtttcagtggcagtggatctgggacagatttcactctcaccatcagcagtctgcaacctgaagattttgctacgtactactgtcaacagcagatggggcgtcctcggacgttcggccaagggaccaaggtggaaatcaaacgg

SEQ ID NO: 318 - protein, 31 aa, Artificial Sequence
  Amino Acid Sequence of Domain Antibody
DIQMTQSPSSLSASVGDRVTITCRASQSIKH

SEQ ID NO: 319 - protein, 76 aa, Artificial sequence
  Domain antibody amino acid sequence
LAWYQQKPGKAPKLLIYKASVLQSGVPSRFSGSGSGTDFTLTISSLQPEDFATYYCQQLRRRPTTFGQGTKVEIKR

SEQ ID NO: 320 - DNA, 324 bp, Artificial Sequence
  Nucleotide Sequence of Domain Antibody
gacatccagatgacccagtctccatcctccctgtctgcatctgtaggagaccgtgtcaccatcacttgccgggcaagtcagagcattaagcattagttagcttggtaccagcagaaaccagggaaagcccctaagctcctgatctataaggcatccgtgttgcaaagtggggtcccatcacgtttcagtggcagtggatctgggacagatttcactctcaccatcagcagtctgcaacctgaagattttgctacgtactactgtcaacagcttaggcgtcgtcctactacgttcggccaagggaccaaggtggaaatcaaacgg

SEQ ID NO: 321 - protein, 31 aa, Artificial Sequence
  Amino Acid Sequence of Domain Antibody
DIQMTQSPSSLSASVGDRVTITCRASQSVKA

SEQ ID NO: 322 - protein, 76 aa, Artificial sequence
  Domain antibody amino acid sequence
LTWYQQKPGKAPKLLIYKASTLQSGVPSRFSGSGSGTDFTLTISSLQPEDFATYYCQQHSSRPYTFGQGTKVEIKR

SEQ ID NO: 323 - DNA, 324 bp, Artificial Sequence
  Nucleotide Sequence of Domain Antibody
gacatccagatgacccagtctccatcctccctgtctgcatctgtaggagaccgtgtcaccatcacttgccgggcaagtcagagcgttaaggcttagttaacttggtaccagcagaaaccagggaaagcccctaagctcctgatctataaggcatccactttgcaaagtggggtcccatcacgtttcagtggcagtggatctgggacagatttcactctcaccatcagcagtctgcaacctgaagattttgctacgtactactgtcaacagcatagttctaggccttatacgttcggccaagggaccaaggtggaaatcaaacgg

SEQ ID NO: 324 - protein, 49 aa, Artificial Sequence
  Amino Acid Sequence of Domain Antibody
DIQMTQSPSSLSASVGDRVTITCRASQSIENRLGEYQQKPGKAPKLLIY

SEQ ID NO: 325 - protein, 58 aa, Artificial sequence
  Domain antibody amino acid sequence
ASLLQSGVPSRFSGSGSGTDFTLTISSLQPEDFATYYCQQDSYFPRTFGQGTKVEIKR

SEQ ID NO: 326 - DNA, 324 bp, Artificial Sequence
  Nucleotide Sequence of Domain Antibody
gacatccagatgacccagtctccatcctccctgtctgcatctgtaggagaccgtgtcaccatcacttgccgggcaagtcagagcattgagaatcggttaggttggtaccagcagaaaccagggaaagcccctaagctcctgatctattaggcgtccttgttgcaaagtggggtcccatcacgtttcagtggcagtggatctgggacagatttcactctcaccatcagcagtctgcaacctgaagattttgctacgtactactgtcaacaggattcgtattttcctcgtacgttcggccaagggaccaaggtggaaatcaaacgg

SEQ ID NO: 327 - protein, 49 aa, Artificial Sequence
  Amino Acid Sequence of Domain Antibody
DIQMTQSPSSLSASVGDRVTITCRASQSIMDKLKWYQQKPGKAPKLLIY

SEQ ID NO: 328 - protein, 58 aa, Artificial sequence
  Domain antibody amino acid sequence
ASILQSGVPSRFSGSGSGTDFTLTISSLQPEDFATYYCQQDSGGPNTFGQGTKVEIKR

SEQ ID NO: 329 - DNA, 324 bp, Artificial Sequence
  Nucleotide Sequence of Domain Antibody
gacatccagatgacccagtctccatcctccctgtctgcatctgtaggagaccgtgtcaccatcacttgccgggcaagtcagagcattatggataagttaaagtggtaccagcagaaaccagggaaagcccctaagctcctgatctattaggcatccattttgcaaagtggggtcccatcacgtttcagtggcagtggatctgggacagatttcactctcaccatcagcagtctgcaacctgaagattttgctacgtactactgtcaacaggatagtgggggtcctaatacgttcggccaagggaccaaggtggaaatcaaacgg

SEQ ID NO: 330 - protein, 108 aa, Artificial Sequence
  Amino Acid Sequence of Domain Antibody
DIQMTQSPSSLSASVGDRVTITCRASQSIGRNLEWYQQKPGKAPKLLIYDASHLQSGVPSRFSGSGSGTDFTLTISSLQPEDFATYYCQQSRELPRTFGQGTKVEIKR

SEQ ID NO: 331 - DNA, 324 bp, Artificial Sequence
  Nucleotide Sequence of Domain Antibody
gacatccagatgacccagtctccatcctccctgtctgcatctgtaggagaccgtgtcaccatcacttgccgggcaagtcagagcattgggaggaatttagagtggtaccagcagaaaccagggaaagcccctaagctcctgatctatgatgcatcccatttgcaaagtggggtcccatcacgtttcagtggcagtggatctgggacagatttcactctcaccatcagcagtctgcaacctgaagattttgctacgtactactgtcaacagtcgcgttggcttcctcgtacgttcggccaagggaccaaggtggaaatcaaacgg

SEQ ID NO: 332 - protein, 108 aa, Artificial Sequence
  Amino Acid Sequence of Domain Antibody
DIQMTQSPSSLSASVGDRVTITCRASQSIRKMLVWYQQKPGKAPKLLIYRASYLQSGVPSRFSGSGSGTDFTLTISSLQPEDFATYYCQQAFRRPRTFGQGTKVEIKR

SEQ ID NO: 333 - DNA, 324 bp, Artificial Sequence
  Nucleotide Sequence of Domain Antibody
gacatccagatgacccagtctccatcctccctgtctgcatctgtaggagaccgtgtcaccatcacttgccgggcaagtcagagcattaggaagatgttagtttggtaccagcagaaaccagggaaagcccctaagctcctgatctatcgggcatcctatttgcaaagtggggtcccatcacgtttcagtggcagtggatctgggacagatttcactctcaccatcagcagtctgcaacctgaagattttgctacgtactactgtcaacaggcttttcggcggcctaggacgttcggccaagggaccaaggtggaaatcaaacgg

SEQ ID NO: 334 - protein, 115 aa, Artificial Sequence
  Amino Acid Sequence of Domain Antibody
EVQLLESGGGLVQPGGSLRLSCAASGFTFDLYNMFWVRQAPGKGLEWVSFISQTGRLTWYADSVKGRFTISRDNSKNTLYLQMNSLRAEDTAVYYCAKTLEDFDYWGQGTLVTVS

SEQ ID NO: 335 - DNA, 345 bp, Artificial Sequence
  Nucleotide Sequence of Domain Antibody
gaggtgcagctgttggagtctgggggaggcttggtacagcctggggggtccctgcgtctctcctgtgcagcctccggattcacctttgatctttataatatgttttgggtccgccaggctccagggaagggtctagagtgggtctcatttattagtcagactggtaggcttacatggtacgcagactccgtgaagggccggttcaccatctcccgcgacaattccaagaacacgctgtatctgcaaatgaacagcctgcgtgccgaggacaccgcggtatattactgtgcgaaaacgctggaggattttgactactggggccagggaaccctggtcaccgtctcg

SEQ ID NO: 336 - protein, 119 aa, Artificial Sequence
  Amino Acid Sequence of Domain Antibody
EVQLLESGGGLVQPGGSLRLSCAASGFTFPVYMMGWVRQAPGKGLEWVSSIDALGGRTGYADSVKGRFTISRDNSKNTLYLQMNSLRAEDTAVYYCAKTMSNKTHTFDYWGQGTLVTVS

SEQ ID NO: 337 - DNA, 357 bp, Artificial Sequence
  Nucleotide Sequence of Domain Antibody
gaggtgcagctgttggagtctgggggaggcttggtacagcctggggggtccctgcgtctctcctgtgcagcctccggattcacctttccggtttatatgatgggttgggtccgccaggctccagggaagggtctagagtgggtctcatcgattgatgctcttggtgggcggacaggttacgcagactccgtgaagggccggttcaccatctcccgcgacaattccaagaacacgctgtatctgcaaatgaacagcctgcgtgccgaggacaccgcggtatattactgtgcgaaaactatgtcgaataagacgcatacgtttgactactggggccagggaaccctggtcaccgtctcg

SEQ ID NO: 338 - protein, 56 aa, Artificial Sequence
  Amino Acid Sequence of Domain Antibody
EVQLLESGGGLVQPGGSLRLSCAASGFTFVAYNMTWVRQAPGKGLEWVSSINTFGN

SEQ ID NO: 339 - protein, 58 aa, Artificial sequence
  Domain antibody amino acid sequence
TRYADSVKGRFTISRDNSKNTLYLQMNSLRAEDTAVYYCAKGSRPFDYWGQGTLVTVS

SEQ ID NO: 340 - DNA, 345 bp, Artificial Sequence
  Nucleotide Sequence of Domain Antibody
gaggtgcagctgttggagtctgggggaggcttggtacagcctggggggtccctgcgtctctcctgtgcagcctccggattcacctttgtggcttataatatgacttgggtccgccaggctccagggaagggtctagagtgggtctcaagtattaatacttttggtaattagacaaggtacgcagactccgtgaagggccggttcaccatctcccgcgacaattccaagaacacgctgtatctgcaaatgaacagcctgcgtgccgaggacaccgcggtatattactgtgcgaaaggtagtaggccttttgactactggggccagggaaccctggtcaccgtctcg

SEQ ID NO: 341 - protein, 29 aa, Artificial Sequence
  Amino Acid Sequence of Domain Antibody
EVQLLESGGGLVQPGGSLRLSCAASGFTF

SEQ ID NO: 342 - protein, 89 aa, Artificial sequence
  Domain antibody amino acid sequence
GYRMGWVRQAPGKGLEWVSWITRTGGTTQYADSVKGRFTISRDNSKNTLYLQMNSLRAEDTAVYYCAKPAKLVGVGFDYWGQGTLVTVS

SEQ ID NO: 343 - DNA, 357 bp, Artificial Sequence
  Nucleotide Sequence of Domain Antibody
gaggtgcagctgttggagtctgggggaggcttggtacagcctggggggtccctgcgtctctcctgtgcagcctccggattcaccttttaggggtatcgtatgggttgggtccgccaggctccagggaagggtctagagtgggtctcatggattacgcgtactggtgggacgacacagtacgcagactccgtgaagggccggttcaccatctcccgcgacaattccaagaacacgctgtatctgcaaatgaacagcctgcgtgccgaggacaccgcggtatattactgtgcgaaaccggcgaagcttgttggggttgggtttgactactggggccagggaaccctggtcaccgtctcg

SEQ ID NO: 344 - protein, 32 aa, Artificial Sequence
  Amino Acid Sequence of Domain Antibody
EVQLLESGGGLVQPGGSLRLSCAASGFTFRKY

SEQ ID NO: 345 - protein, 86 aa, Artificial sequence
  Domain antibody amino acid sequence
MGWVRQAPGKGLEWVSQIGAKGQSTDYADSVKGRFTISRDNSKNTLYLQMNSLRAEDTAVYYCAKKKRGENYFFDYWGQGTLVTVS

SEQ ID NO: 346 - DNA, 357 bp, Artificial Sequence
  Nucleotide Sequence of Domain Antibody
gaggtgcagctgttggagtctgggggaggcttggtacagcctggggggtccctgcgtctctcctgtgcagcctccggattcacctttcggaagtattagatggggtgggtccgccaggctccagggaagggtctagagtgggtctcacagattggtgcgaagggtcagtctacagattacgcagactccgtgaagggccggttcaccatctcccgcgacaattccaagaacacgctgtatctgcaaatgaacagcctgcgtgccgaggacaccgcggtatattactgtgcgaaaaagaagaggggggagaattatttttttgactactggggccagggaaccctggtcaccgtctcg

SEQ ID NO: 347 - protein, 119 aa, Artificial Sequence
  Amino Acid Sequence of Domain Antibody
EVQLLESGGGLVQPGGSLRLSCAASGFTFRRYSMSWVRQAPGKGLEWVSDISRSGRYTHYADSVKGRFTISRDNSKNTLYLQMNSLRAEDTAVYYCAKRIDSSQNGFDYWGQGTLVTVS

SEQ ID NO: 348 - DNA, 357 bp, Artificial Sequence
  Nucleotide Sequence of Domain Antibody
gaggtgcagctgttggagtctgggggaggcttggtacagcctggggggtccctgcgtctctcctgtgcagcctccggattcacctttcggcggtatagtatgtcgtgggtccgccaggctccagggaagggtctagagtgggtctcagatatttctcgttctggtcggtatacacattacgcagactccgtgaagggccggttcaccatctcccgcgacaattccaagaacacgctgtatctgcaaatgaacagcctgcgtgccgaggacaccgcggtatattactgtgcgaaacgtattgattcttctcagaatgggtttgactactggggccagggaaccctggtcaccgtctcg

SEQ ID NO: 349 - protein, 29 aa, Artificial Sequence
  Amino Acid Sequence of Domain Antibody
EVQLLESGGGLVQPGGSLRLSCAASGFTF

SEQ ID NO: 350 - protein, 85 aa, Artificial sequence
  Domain antibody amino acid sequence
GYKMFWVRQAPGKGLEWVSAISGSGGSTYYADSVKGRFTISRDNSKNTLYLQMNSLRAEDTAVYYCAKQKENFDYWGQGTLVTVS

SEQ ID NO: 351 - DNA, 345 bp, Artificial Sequence
  Nucleotide Sequence of Domain Antibody
gaggtgcagctgttggagtctgggggaggcttggtacagcctggggggtccctgcgtctctcctgtgcagcctccggattcaccttttaggggtataagatgttttgggtccgccaggctccagggaagggtctagagtgggtctcagctattagtggtagtggtggtagcacatactacgcagactccgtgaagggccggttcaccatctcccgcgacaattccaagaacacgctgtatctgcaaatgaacagcctgcgtgccgaggacaccgcggtatattactgtgcgaaacagaaggagaattttgactactggggccagggaaccctggtcaccgtctcg

SEQ ID NO: 352 - protein, 119 aa, Artificial Sequence
  Amino Acid Sequence of Domain Antibody
EVQLLESGGGLVQPGGSLRLSCAASGFTFGDYAMWWVRQAPGKGLEWVSVISSNGGSTFYADSVKGRFTISRDNSKNTLYLQMNSLRAEDTAVYYCAKRVRKRTPEFDYWGQGTLVTVS

SEQ ID NO: 353 - DNA, 357 bp, Artificial Sequence
  Nucleotide Sequence of Domain Antibody
gaggtgcagctgttggagtctgggggaggcttggtacagcctggggggtccctgcgtctctcctgtgcagcctccggattcacctttggggattatgctatgtggtgggtccgccaggctccagggaagggtctagagtgggtctcagtgattagttcgaatggtgggagtacattttacgcagactccgtgaagggccggttcaccatctcccgcgacaattccaagaacacgctgtatctgcaaatgaacagcctgcgtgccgaggacaccgcggtatattactgtgcgaaacgtgttcgtaagaggactcctgagtttgactactggggccagggaaccctggtcaccgtctcg

SEQ ID NO: 354 - protein, 119 aa, Artificial Sequence
  Amino Acid Sequence of Domain Antibody
EVQLLESGGGLVQPGGSLRLSCAASGFTFRRYKMGWVRQAPGKGLEWVSAIGRNGTKTNYADSVKGRFTISRDNSKNTLYLQMNSLRAEDTAVYYCAKIYTGKPAAFDYWGQGTLVTVS

SEQ ID NO: 355 - DNA, 357 bp, Artificial Sequence
  Nucleotide Sequence of Domain Antibody
gaggtgcagctgttggagtctgggggaggcttggtacagcctggggggtccctgcgtctctcctgtgcagcctccggattcacctttaggaggtataagatgggttgggtccgccaggctccagggaagggtctagagtgggtctcagcgattgggaggaatggtacgaagacaaattacgcagactccgtgaagggccggttcaccatctcccgcgacaattccaagaacacgctgtatctgcaaatgaacagcctgcgtgccgaggacaccgcggtatattactgtgcgaaaatttatacggggaagcctgctgcgtttgactactggggccagggaaccctggtcaccgtctcg

SEQ ID NO: 356 - protein, 32 aa, Artificial Sequence
  Amino Acid Sequence of Domain Antibody
EVQLLESGGGLVQPGGSLRLSCAASGFTFKKY

SEQ ID NO: 357 - protein, 86 aa, Artificial sequence
  Domain antibody amino acid sequence
MSWVRQAPGKGLEWVSAISGSGGSTYYADSVKGRFTISRDNSKNTLYLQMNSLRAEDTAVYYCAKMLRTKNKVFDYWGQGTLVTVS

SEQ ID NO: 358 - DNA, 357 bp, Artificial Sequence
  Nucleotide Sequence of Domain Antibody
gaggtgcagctgttggagtctgggggaggcttggtacagcctggggggtccctgcgtctctcctgtgcagcctccggattcacctttaagaagtattagatgtcttgggtccgccaggctccagggaagggtctagagtgggtctcagctattagtggtagtggtggtagcacatactacgcagactccgtgaagggccggttcaccatctcccgcgacaattccaagaacacgctgtatctgcaaatgaacagcctgcgtgccgaggacaccgcggtatattactgtgcgaaaatgctgaggactaagaataaggtgtttgactactggggccagggaaccctggtcaccgtctcg

SEQ ID NO: 359 - protein, 119 aa, Artificial Sequence; identical to SEQ ID NO: 354
  Amino Acid Sequence of Domain Antibody

SEQ ID NO: 360 - DNA, 357 bp, Artificial Sequence; identical to SEQ ID NO: 355
  Nucleotide Sequence of Domain Antibody

SEQ ID NO: 361 - protein, 29 aa, Artificial Sequence
  Amino Acid Sequence of Domain Antibody
EVQLLESGGGLVQPGGSLRLSCAASGFTF

SEQ ID NO: 362 - protein, 89 aa, Artificial sequence
  Domain antibody amino acid sequence
SYRMGWVRQAPGKGLEWVSSISSRGRHTSYADSVKGRFTISRDNSKNTLYLQMNSLRAEDTAVYYCAKRVPGRGRSFDYWGQGTLVTVS

SEQ ID NO: 363 - DNA, 357 bp, Artificial Sequence
  Nucleotide Sequence of Domain Antibody
gaggtgcagctgttggagtctgggggaggcttggtacagcctggggggtccctgcgtctctcctgtgcagcctccggattcaccttttagagttatcggatgggttgggtccgccaggctccagggaagggtctagagtgggtctcaagtatttcgtcgaggggtaggcatacatcttacgcagactccgtgaagggccggttcaccatctcccgcgacaattccaagaacacgctgtatctgcaaatgaacagcctgcgtgccgaggacaccgcggtatattactgtgcgaaaagggttccgggtcgggggcgttcttttgactactggggccagggaaccctggtcaccgtctcg

SEQ ID NO: 364 - protein, 119 aa, Artificial Sequence
  Amino Acid Sequence of Domain Antibody
EVQLLESGGGLVQPGGSLRLSCAASGFPFRRYRMRWVRQAPGKGLEWVSGISPGGKHTTYADSVKGRFTISRDNSKNTLYLQMNSLRAEDTAVYYCAKGEGGASSAFDYWGQGTLVTVS

SEQ ID NO: 365 - DNA, 357 bp, Artificial Sequence
  Nucleotide Sequence of Domain Antibody
gaggtgcagctgttggagtctgggggaggcttggtacagcctggggggtccctgcgtctctcctgtgcagcctccggattcccctttcgtcggtatcggatgaggtgggtccgccaggctccagggaagggtctagagtgggtctcaggtatttctccgggtggtaagcatacaacgtacgcagactccgtgaagggccggttcaccatctcccgcgacaattccaagaacacgctgtatctgcaaatgaacagcctgcgtgccgaggacaccgcggtatattactgtgcgaaaggtgaggggggggcgagttctgcgtttgactactggggccagggaaccctggtcaccgtctcg

SEQ ID NO: 366 - protein, 29 aa, Artificial Sequence
  Amino Acid Sequence of Domain Antibody
EVQLLESGGGLVQPGGSLRLSCAASGFTF

SEQ ID NO: 367 - protein, 89 aa, Artificial sequence
  Domain antibody amino acid sequence
RYGMVWVRQAPGKGLEWVSAISGSGGSTYYADSVKGRFTISRDNSKNTLYLQMNSLRAEDTAVYYCAKRHSSEARQFDYWGQGTLVTVS

SEQ ID NO: 368 - DNA, 357 bp, Artificial Sequence
  Nucleotide Sequence of Domain Antibody
gaggtgcagctgttggagtctgggggaggcttggtacagcctggggggtccctgcgtctctcctgtgcagcctccggattcaccttttagcggtatgggatggtttgggtccgccaggctccagggaagggtctagagtgggtctcagctattagtggtagtggtggtagcacatactacgcagactccgtgaagggccggttcaccatctcccgcgacaattccaagaacacgctgtatctgcaaatgaacagcctgcgtgccgaggacaccgcggtatattactgtgcgaaacggcatagttctgaggctaggcagtttgactactggggccagggaaccctggtcaccgtctcg